(19) United States
(12) Patent Application Publication
(10) Pub. No.: US 2023/0416205 A1
(43) Pub. Date: Dec. 28, 2023

(54) COMPOUNDS, COMPOSITIONS, AND METHODS

(71) Applicant: Denali Therapeutics Inc, South San Francisco, CA (US)

(72) Inventors: Alex L. Bagdasarian, South San Francisco, CA (US); Cyril Bucher, San Mateo, CA (US); Robert A. Craig, II, South San Francisco, CA (US); Javier de Vicente Fidalgo, Foster City, CA (US); Anthony A. Estrada, Danville, CA (US); Brian M. Fox, Redwood City, CA (US); Cheng Hu, San Mateo, CA (US); Benjamin J. Huffman, Brisbane, CA (US); Katrina W. Lexa, San Francisco, CA (US); Lizanne G. Nilewski, Pacifica, CA (US); Maksim Osipov, Redwood City, CA (US); Arun Thottumkara, San Francisco, CA (US)

(21) Appl. No.: 18/037,999

(22) PCT Filed: Nov. 19, 2021
(86) PCT No.: PCT/US2021/060088

§ 371 Date: May 19, 2023

Related U.S. Application Data
(60) Provisional application No. 63/256,393, filed on Oct. 15, 2021.
(60) Provisional application No. 63/182,741, filed on Apr. 30, 2021.
(60) Provisional application No. 63/127,928, filed on Dec. 18, 2020.
(60) Provisional application No. 63/116,727, filed on Nov. 20, 2020.

(51) Int. Cl. C07D 217/24; C07D 401/12; C07D 471/04; C07D 401/14; C07D 493/08; C07D 491/107; C07D 413/08; C07D 405/12; C07D 417/12; C07D 487/04; C07D 417/14; C07D 498/04; C07D 405/14; C07D 495/04

(52) U.S. Cl. CPC C07D 217/24; C07D 401/12; C07D 471/04; C07D 401/14; C07D 493/08; C07D 491/107; C07D 413/08; C07D 405/12; C07D 417/12; C07D 487/04; C07D 417/14; C07D 498/04; C07D 405/14; C07D 495/04

(57) ABSTRACT

The present disclosure relates generally to small molecule modulators of NLR Family Pyrin Domain Containing 3 (NL-RP3), or a pharmaceutically acceptable salt, isotopically enriched analog, stereoisomer, mixture of stereoisomers, or prodrug thereof, methods of making and intermediates thereof, and methods of using thereof.

### CROSS REFERENCE TO RELATED APPLICATIONS

This application claims the benefit under 35 U.S.C. § 119(e) to U.S. Provisional Application No. 63/116,727, filed Nov. 20, 2020, 63/127,928, filed Dec. 18, 2020, 63/182,741, filed Apr. 30, 2021, and 63/256,393, filed Oct. 15, 2021, each of which is incorporated by reference in its entirety.

### FIELD

The present disclosure relates generally to small molecule modulators of NLR Family Pyrin Domain Containing 3 (NLRP3), and their use as therapeutic agents.

### BACKGROUND

Inhibition of NLRP3 activation has been shown to result in potent therapeutic effects in animal models of inflammatory diseases. Modulators of NLRP3, inhibitors in particular, have broad therapeutic potential in a wide array of auto-inflammatory and chronic inflammatory diseases that either require better treatment options or for which no adequate therapies exist. Therapies targeting NLRP3-dependent cytokines are already approved for therapeutic use; however, they have notable disadvantages relative to direct NLRP3 antagonists. There remains a strong impetus for the discovery and clinical development of molecules that antagonize NLRP3.

### DESCRIPTION

Provided herein are compounds, or a pharmaceutically acceptable salt, isotopically enriched analog, stereoisomer, mixture of stereoisomers, or prodrug thereof, that are useful in treating and/or preventing diseases mediated, at least in part, by NLRP3.

In some embodiments, provided are compounds that modulate the activity of NLRP3. In some embodiments, the compounds inhibit the activation of NLRP3.

In another embodiment, provided is a pharmaceutical composition comprising a compound as described herein, or a pharmaceutically acceptable salt, isotopically enriched analog, stereoisomer, mixture of stereoisomers, or prodrug thereof, and a pharmaceutically acceptable carrier.

In another embodiment, provided is a method for treating a disease or condition mediated, at least in part, by NLRP3, the method comprising administering an effective amount of the pharmaceutical composition comprising a compound as described herein, or a pharmaceutically acceptable salt, isotopically enriched analog, stereoisomer, mixture of stereoisomers, or prodrug thereof.

In another embodiment, provided is a method for treating a disease or condition mediated, at least in part, by TNF-α, the method comprising administering an effective amount of the pharmaceutical composition comprising a compound as described herein, or a pharmaceutically acceptable salt, isotopically enriched analog, stereoisomer, mixture of stereoisomers, or prodrug thereof. In some embodiments the administration is to a subject resistant to treatment with an anti-TNF-αagent. In some embodiments, the disease is a gut disease or condition. In some embodiments the disease or condition is inflammatory bowel disease, Crohn's disease, or ulcerative colitis.

The disclosure also provides compositions, including pharmaceutical compositions, kits that include the compounds, or a pharmaceutically acceptable salt, isotopically enriched analog, stereoisomer, mixture of stereoisomers, or prodrug thereof, methods of using (or administering) and making the compounds, or a pharmaceutically acceptable salt, isotopically enriched analog, stereoisomer, mixture of stereoisomers, or prodrug thereof, and intermediates thereof.

The disclosure further provides compounds, or a pharmaceutically acceptable salt, isotopically enriched analog, stereoisomer, mixture of stereoisomers, or prodrug thereof, or compositions thereof for use in a method of treating a disease, disorder, or condition that is mediated, at least in part, by NLRP3.

Moreover, the disclosure provides uses of the compounds, or a pharmaceutically acceptable salt, isotopically enriched analog, stereoisomer, mixture of stereoisomers, or prodrug thereof, or compositions thereof in the manufacture of a medicament for the treatment of a disease, disorder, or condition that is mediated, at least in part, by NLRP3.

The description herein sets forth exemplary embodiments of the present technology. It should be recognized, however, that such description is not intended as a limitation on the scope of the present disclosure but is instead provided as a description of exemplary embodiments.

### 1. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through a line in a structure indicates a specified point of attachment of a group. Unless chemically or structurally required, no directionality or stereochemistry is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂), and tert-butyl (i.e., —C(CH₃)₃); and “propyl” includes n-propyl (i.e., —(CH₂)₂CH₃), and isopropyl (i.e., —CH(CH₃)₂).

Certain commonly used alternative chemical names may be used. For example, a divalent group such as a divalent “alkyl” group, a divalent “aryl” group, a divalent heteroaryl group, etc., may also be referred to as an “alkylene” group or an “alkylenyl” group (for example, methylenyl, ethylenyl, and propylenyl), an “arylene” group or an “arylenyl” group (for example, phenylenyl or napthylenyl, or quinolinyl for heteroarylene), respectively. Also, unless indicated explicitly otherwise, where combinations of groups are referred to herein as one moiety, e.g., arylalkyl or aralkyl, the last mentioned group contains the atom by which the moiety is attached to the rest of the molecule.

“Alkenyl” refers to an alkyl group containing at least one (e.g., 1-3, or 1) carbon-carbon double bond and having from 2 to 20 carbon atoms (i.e., C₂₋₂₀ alkenyl), 2 to 12 carbon atoms (i.e., C₂₋₁₂ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include, e.g., ethenyl, propenyl, butadienyl (including 1,2-butadienyl, and 1,3-butadienyl).

“Alkynyl” refers to an alkyl group containing at least one (e.g., 1-3, or 1) carbon-carbon triple bond and having from 2 to 20 carbon atoms (i.e., C₂₋₂₀ alkynyl), 2 to 12 carbon atoms (i.e., C₂₋₁₂ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Alkoxy” refers to the group “alkyl-O—”. Examples of alkoxy groups include, e.g., methoxy, ethoxy, n-propoxy, iso-propoxy, n-butoxy, tert-butoxy, sec-butoxy, n-pentoxy, n-hexoxy, and 1,2-dimethylbutoxy.

“Alkoxyalkyl” refers to the group “alkyl-O-alkyl”.

“Alkylthio” refers to the group “alkyl-S—”. “Alkylsulfinyl” refers to the group “alkyl-S(O)—”. “Alkylsulfonyl” refers to the group “alkyl-S(O)₂—”. “Alkylsulfonylalkyl” refers to -alkyl-S(O)₂-alkyl.

“Acyl” refers to a group —C(O)R^{y}, wherein R^{y} is hydrogen, alkyl, alkenyl, alkynyl, cycloalkyl, heterocyclyl, aryl, heteroalkyl, or heteroaryl; each of which may be optionally substituted, as defined herein. Examples of acyl include, e.g., formyl, acetyl, cyclohexylcarbonyl, cyclohexylmethyl-carbonyl, and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein, or R^{y} and R^{z} are taken together to form a cycloalkyl or heterocyclyl; each of which may be optionally substituted, as defined herein.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Amidino” refers to —C(NR^{y})(NR^{z}₂), wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, e.g., phenyl, naphthyl, fluorenyl, and anthryl. Aryl, however, does not encompass or overlap in any way with heteroaryl defined below. If one or more aryl groups are fused with a heteroaryl, the resulting ring system is heteroaryl regardless of point of attachment. If one or more aryl groups are fused with a heterocyclyl, the resulting ring system is heterocyclyl regardless of point of attachment. If one or more aryl groups are fused with a cycloalkyl, the resulting ring system is cycloalkyl regardless of point of attachment.

“Arylalkyl” or “Aralkyl” refers to the group “aryl-alkyl-”.

“Carbamoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Carboxyl ester” or “ester” refer to both —OC(O)R^{x} and —C(O)OR^{x}, wherein R^{x} is alkyl, alkenyl, alkynyl, cycloalkyl, heterocyclyl, aryl, heteroalkyl, or heteroaryl; each of which may be optionally substituted, as defined herein.

“Cyanoalkyl” refers to refers to an alkyl group as defined above, wherein one or more (e.g., 1 or 2) hydrogen atoms are replaced by a cyano (—CN) group.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl), 3 to 14 ring carbon atoms (i.e., C₃₋₁₂ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Monocyclic groups include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl, and the like. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Cycloalkylalkyl” refers to the group “cycloalkyl-alkyl-”.

“Imino” refers to a group —C(NR^{y})R^{z}, wherein R^{y} and R^{z} are each independently hydrogen, alkyl, alkenyl, alkynyl, cycloalkyl, heterocyclyl, aryl, heteroalkyl, or heteroaryl; each of which may be optionally substituted, as defined herein.

“Imido” refers to a group —C(O)NR^{y}C(O)R^{z}, wherein R^{y} and R^{z} are each independently hydrogen, alkyl, alkenyl, alkynyl, cycloalkyl, heterocyclyl, aryl, heteroalkyl, or heteroaryl; each of which may be optionally substituted, as defined herein.

“Halogen” or “halo” refers to atoms occupying group VIIA of the periodic table, such as fluoro, chloro, bromo, or iodo.

“Haloalkyl” refers to an unbranched or branched alkyl group as defined above, wherein one or more (e.g., 1 to 6 or 1 to 3) hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Haloalkoxy” refers to an alkoxy group as defined above, wherein one or more (e.g., 1 to 6 or 1 to 3) hydrogen atoms are replaced by a halogen.

“Haloalkoxyalkyl” refers to an alkoxyalkyl group as defined above, wherein one or more (e.g., 1 to 6 or 1 to 3) hydrogen atoms are replaced by a halogen.

“Hydroxyalkyl” refers to an alkyl group as defined above, wherein one or more (e.g., 1 to 6 or 1 to 3) hydrogen atoms are replaced by a hydroxy group.

“Heteroalkyl” refers to an alkyl group in which one or more of the carbon atoms (and any associated hydrogen atoms), excluding any terminal carbon atom(s), are each independently replaced with the same or different heteroatomic group, provided the point of attachment to the remainder of the molecule is through a carbon atom. The term “heteroalkyl” includes unbranched or branched saturated chain having carbon and heteroatoms. By way of example, 1, 2, or 3 carbon atoms may be independently replaced with the same or different heteroatomic group. Heteroatomic groups include, but are not limited to, —NR^{y}—, —O—, —S—, —S(O)—, —S(O)₂—, and the like, wherein R^{y} is hydrogen, alkyl, alkenyl, alkynyl, cycloalkyl, heterocyclyl, aryl, heteroalkyl, or heteroaryl; each of which may be optionally substituted, as defined herein. Examples of heteroalkyl groups include, e.g., ethers (e.g., —CH₂OCH₃, —CH(CH₃)OCH₃, —CH₂CH₂OCH₃, —CH₂CH₂OCH₂CH₂OCH₃, etc.), thioethers (e.g., —CH₂SCH₃, —CH(CH₃)SCH₃, —CH₂CH₂SCH₃, —CH₂CH₂SCH₂CH₂SCH₃, etc.), sulfones (e.g., —CH₂S(O)₂CH₃, —CH(CH₃)S(O)₂CH₃, —CH₂CH₂S(O)₂CH₃, —CH₂CH₂S(O)₂CH₂CH₂OCH₃, etc.), and amines (e.g., —CH₂NR^{y}CH₃, —CH(CH₃)NR^{y}CH₃, —CH₂CH₂NR^{y}CH₃, —CH₂CH₂NR^{y}CH₂CH₂NR^{y}CH₃, etc., where R^{y} is hydrogen, alkyl, alkenyl, alkynyl, cycloalkyl, heterocyclyl, aryl, heteroalkyl, or heteroaryl; each of which may be optionally substituted, as defined herein). As used herein, heteroalkyl includes 2 to 10 carbon atoms, 2 to 8 carbon atoms, or 2 to 4 carbon atoms; and 1 to 3 heteroatoms, 1 to 2 heteroatoms, or 1 heteroatom.

“Heteroaryl” refers to an aromatic group having a single ring or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen, and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen, and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heteroarylalkyl” refers to the group “heteroaryl-alkyl-”.

“Heterocyclyl” refers to a saturated or partially unsaturated cyclic alkyl group, with one or more ring heteroatoms independently selected from nitrogen, oxygen, and sulfur. The term “heterocyclyl” includes heterocycloalkenyl groups (i.e., the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro, and may comprise one or more (e.g., 1 to 3) oxo (═O) or N-oxide (—O—) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to a cycloalkyl, an aryl, or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur, or oxygen. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), thiomorpholinyl, thiamorpholinyl, 1-oxo-thiomorpholinyl, and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

“Heterocyclylalkyl” refers to the group “heterocyclyl-alkyl-.”

“Oxime” refers to the group —CR^{y}(═NOH) wherein R^{y} is hydrogen, alkyl, alkenyl, alkynyl, cycloalkyl, heterocyclyl, aryl, heteroalkyl, or heteroaryl; each of which may be optionally substituted, as defined herein.

“Sulfonyl” refers to the group —S(O)₂R^{y}, where R^{y} is hydrogen, alkyl, alkenyl, alkynyl, cycloalkyl, heterocyclyl, aryl, heteroalkyl, or heteroaryl; each of which may be optionally substituted, as defined herein. Examples of sulfonyl are methylsulfonyl, ethylsulfonyl, phenylsulfonyl, and toluenesulfonyl.

“Sulfinyl” refers to the group —S(O)R^{y}, where R^{y} is hydrogen, alkyl, alkenyl, alkynyl, cycloalkyl, heterocyclyl, aryl, heteroalkyl, or heteroaryl; each of which may be optionally substituted, as defined herein. Examples of sulfinyl are methylsulfinyl, ethylsulfinyl, phenylsulfinyl, and toluenesulfinyl.

“Sulfonamido” refers to the groups —SO₂NR^{y}R^{z} and —NR^{y}SO₂R^{z}, where R^{y} and R^{z} are each independently hydrogen, alkyl, alkenyl, alkynyl, cycloalkyl, heterocyclyl, aryl, heteroalkyl, or heteroaryl; each of which may be optionally substituted, as defined herein.

The terms “optional” or “optionally” means that the subsequently described event or circumstance may or may not occur and that the description includes instances where said event or circumstance occurs and instances in which it does not. Also, the term “optionally substituted” refers to any one or more (e.g., 1 to 5 or 1 to 3) hydrogen atoms on the designated atom or group may or may not be replaced by a moiety other than hydrogen.

The term “substituted” used herein means any of the above groups (i.e., alkyl, alkenyl, alkynyl, alkylene, alkoxy, haloalkyl, haloalkoxy, cycloalkyl, aryl, heterocyclyl, heteroaryl, and/or heteroalkyl) wherein at least one (e.g., 1 to 5 or 1 to 3) hydrogen atom is replaced by a bond to a non-hydrogen atom such as, but not limited to alkyl, alkenyl, alkynyl, alkoxy, alkylthio, acyl, amido, amino, amidino, aryl, aralkyl, azido, carbamoyl, carboxyl, carboxyl ester, cyano, cycloalkyl, cycloalkylalkyl, guanadino, halo, haloalkyl, haloalkoxy, hydroxyalkyl, heteroalkyl, heteroaryl, heteroarylalkyl, heterocyclyl, heterocyclylalkyl, —NHNH₂, ═NNH₂, imino, imido, hydroxy, oxo, oxime, nitro, sulfonyl, sulfinyl, alkylsulfonyl, alkylsulfinyl, thiocyanate, —S(O)OH, —S(O)₂OH, sulfonamido, thiol, thioxo, N-oxide, or —Si(R^{y})₃, wherein each R^{y} is independently hydrogen, alkyl, alkenyl, alkynyl, heteroalkyl, cycloalkyl, aryl, heteroaryl, or heterocyclyl.

In certain embodiments. “substituted” includes any of the above alkyl, alkenyl, alkynyl, cycloalkyl, heterocyclyl, aryl, or heteroaryl groups in which one or more (e.g., 1 to 5 or 1 to 3) hydrogen atoms are independently replaced with deuterium, halo, cyano, nitro, azido, oxo, alkyl, alkenyl, alkynyl, haloalkyl, cycloalkyl, heterocyclyl, aryl, heteroaryl, —NR^{g}R^{h}, —NR^{g}C(O)R^{h}, —NR^{g}C(O)NR^{g}R^{h}, —NR^{g}C(O)OR^{h}, —NR^{g}S(O)₁₋₂R^{h}, —C(O)R^{g}, —C(O)OR^{g}, —OC(O)OR^{g}, —OC(O)R^{g}, —C(O)NR^{g}R^{h}, —OC(O)NR^{g}R^{h}, —OR^{g}, —SR^{g}, —S(O)R^{g}, —S(O)₂R^{g}, —OS(O)₁₋₂R^{g}, —S(O)₁₋₂OR^{g}, —NR^{g}S(O)₁₋₂NR^{g}R^{h}, ═NSO₂R^{g}, ═NOR^{g}, —S(O)₁₋₂NR^{g}R^{h}, —SF₅, —SCF₃, or —OCF₃. In certain embodiments, “substituted” also means any of the above groups in which one or more (e.g., 1 to 5 or 1 to 3) hydrogen atoms are replaced with —C(O)R^{g}, —C(O)OR^{g}, —C(O)NR^{g}R^{h}, —CH₂SO₂R^{g}, or —CH₂SO₂NR^{g}R^{h}. In the foregoing, R^{g} and R^{h} are the same or different and independently hydrogen, alkyl, alkenyl, alkynyl, alkoxy, thioalkyl, aryl, aralkyl, cycloalkyl, cycloalkylalkyl, haloalkyl, heterocyclyl, heterocyclylalkyl, heteroaryl, and/or heteroarylalkyl. In certain embodiments, “substituted” also means any of the above groups in which one or more (e.g., 1 to 5 or 1 to 3) hydrogen atoms are replaced by a bond to an amino, cyano, hydroxy, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, and/or heteroarylalkyl, or two of R^{g} and R^{h} and Rⁱ are taken together with the atoms to which they are attached to form a heterocyclyl ring optionally substituted with oxo, halo, or alkyl optionally substituted with oxo, halo, amino, hydroxy, or alkoxy.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein.

In certain embodiments, as used herein, the phrase “one or more” refers to one to five. In certain embodiments, as used herein, the phrase “one or more” refers to one to three.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵, respectively. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life, reduced dosage requirements, and/or an improvement in therapeutic index. An ¹⁸F, ³H, ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino, and/or carboxyl groups, or groups similar thereto.

Provided are also or a pharmaceutically acceptable salt, isotopically enriched analog, deuterated analog, stereoisomer, mixture of stereoisomers, and prodrugs of the compounds described herein. “Pharmaceutically acceptable” or “physiologically acceptable” refer to compounds, salts, compositions, dosage forms, and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids, and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic or organic bases. Salts derived from inorganic bases include, by way of example only, sodium, potassium, lithium, aluminum, ammonium, calcium, and magnesium salts. Salts derived from organic bases include, but are not limited to, salts of primary, secondary, and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds of the disclosure, or their pharmaceutically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and/or fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present disclosure contemplates various stereoisomers, or mixtures thereof, and includes “enantiomers,” which refers to two stereoisomers whose molecules are nonsuperimposeable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

“Prodrugs” means any compound which releases an active parent drug according to a structure described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs may be prepared by modifying functional groups present in the compounds in such a way that the modifications are cleaved, either in routine manipulation or in vivo, to the parent compounds. Prodrugs include compounds described herein wherein a hydroxy, amino, carboxyl, or sulfhydryl group in a compound described herein is bonded to any group that may be cleaved in vivo to regenerate the free hydroxy, amino, or sulfhydryl group, respectively. Examples of prodrugs include, but are not limited to esters (e.g., acetate, formate, and benzoate derivatives), amides, guanidines, carbamates (e.g., N,N-dimethylaminocarbonyl) of hydroxy functional groups in compounds described herein, and the like. Preparation, selection, and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

### 2. Compounds

Provided herein are compounds that are modulators of NLRP3. In certain embodiments, provided is a compound of Formula I:

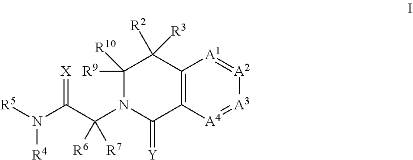

or a pharmaceutically acceptable salt, isotopically enriched analog, stereoisomer, mixture of stereoisomers, or prodrug thereof, wherein:
X is O or S;
Y is O or S;
A¹, A², A³, and A⁴ are each independently N, CH, or CR¹; provided at least one of A¹, A², A³, and A⁴ is CR¹;
each R¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —C(O)R¹¹, —C(O)OR¹¹, —S(O)₀₋₂R¹¹, —NR¹¹S(O)₀₋₂—R¹¹, —S(O)₀₋₂N(R¹¹)₂, —NR¹¹S(O)₀₋₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹; or
any two adjacent R¹ together with the atoms to which they are attached form a cycloalkyl, heterocyclyl, aryl, or heteroaryl ring; wherein the cycloalkyl, heterocyclyl, aryl, or heteroaryl is independently optionally substituted with one to eight Z¹;
R² is C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, —NO₂, —SF₅, —OR¹¹, —N(R¹¹)₂, —C(O)R¹¹, —C(O)OR¹¹, —S(O)₀₋₂—R¹¹, —NR¹¹S(O)₀₋₂—R¹¹, —S(O)₀₋₂N(R¹¹)₂, —NR¹¹S(O)₀₋₂N(R¹¹)₂, —NR¹¹C(O)N(R¹¹)₂, —NR¹¹C(O)OR¹¹, —NR¹¹C(O)R¹¹, —OC(O)R¹¹, —OC(O)N(R¹¹)₂, —C(O)N(R¹¹)₂, halo, or cyano; wherein the C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is optionally substituted with one to eight Z¹;
R³ is hydrogen, halo, cyano, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is optionally substituted with one to eight Z¹; or
R² and R³ together form a C₃₋₁₀ cycloalkyl or heterocyclyl ring; wherein the C₃₋₁₀ cycloalkyl or heterocyclyl is optionally substituted with one to eight Z¹;
R⁴ is hydrogen, C₁₋₆ alkyl, C₂₋₆ alkenyl, C₂₋₆ alkynyl. C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is optionally substituted with one to eight Z¹; or
R⁵ is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is optionally substituted with one to eight Z¹; or
R⁴ and R⁵ together form a heterocyclyl or heteroaryl ring optionally substituted with one to eight Z¹;
R⁶ is hydrogen, halo, cyano, C₁₋₆ alkyl, C₂₋₆ alkenyl, C₂₋₆ alkynyl, C₁₋₆ haloalkyl, C₂₋₆ heteroalkyl, C₃₋₁₀ cycloalkyl, or heterocyclyl; wherein the C₁₋₆ alkyl, C₂₋₆ alkenyl, C₂₋₆ alkynyl, C₁₋₆ haloalkyl, C₂₋₆ heteroalkyl, C₃₋₁₀ cycloalkyl, or heterocyclyl may further be optionally substituted with one to five Z^{1b};
R⁷ is hydrogen, halo, cyano, hydroxy, C₁₋₆ alkyl, C₂₋₆ alkenyl, C₂₋₆ alkynyl, C₁₋₆ haloalkyl, C₂₋₆ heteroalkyl, C₃₋₁₀ cycloalkyl, or heterocyclyl; wherein the C₁₋₆ alkyl, C₂₋₆ alkenyl, C₂₋₆ alkynyl, C₁₋₆ haloalkyl, C₂₋₆ heteroalkyl, C₃₋₁₀ cycloalkyl, or heterocyclyl, or may further be optionally substituted with one to five Z^{1b};
or R⁶ and R⁷ join to form a C₃₋₁₀ cycloalkyl or heterocyclyl ring; wherein the C₃₋₁₀ cycloalkyl or heterocyclyl ring may further be optionally substituted with one to five Z^{1b};
R⁹ and R¹⁰ are each independently hydrogen, halo, cyano, C₁₋₆ alkyl, or C₁₋₆ haloalkyl, wherein each C₁₋₆ alkyl or C₁₋₆ haloalkyl is independently optionally substituted with one to five Z¹; or
R⁹ and R¹⁰ together form a C₃₋₁₀ cycloalkyl or heterocyclyl ring; wherein the C₃₋₁₀ cycloalkyl or heterocyclyl is optionally substituted with one to eight Z¹;
each Z¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —C(O)R¹¹, —C(O)OR¹¹, —S(O)₀₋₂R¹¹, —NR¹¹S(O)₀₋₂—R¹¹, —S(O)₀₋₂N(R¹¹)₂, —NR¹¹S(O)₀₋₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each R¹¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹¹ is independently optionally substituted with one to five Z^{1a};
each Z^{1a} is independently hydroxy, halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, —N(R¹³)₂, —OR¹³, —C(O)R¹³, —C(O)OR¹³, —S(O)₀₋₂R¹³, —NR¹³S(O)₀₋₂—R¹³, —S(O)₀₋₂N(R¹³)₂, —NR¹³S(O)₀₋₂N(R¹³)₂, —NR ¹³C(O)N(R¹³)₂, —C(O)N(R¹³)₂, —NR¹³C(O)R¹³, —OC(O)N(R¹³)₂, or —NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₁₋₆haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹³ is independently optionally substituted with one to five Z^{1b};
each Z^{1b} is independently halo, cyano, hydroxy, —SH, —NH₂, —NO₂, —SF₅, C₁₋₆ alkyl, C₂₋₆alkenyl, C₂₋₆ alkynyl, C₁₋₆ haloalkyl, C₃₋₁₀ cycloalkyl, heterocyclyl, aryl, heteroaryl, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, —N(C₁₋₆ alkyl)-, —N(C₂₋₆ alkenyl)-, —N(C₂₋₆ alkynyl)-, —N(C₁₋₆ haloalkyl)-, —N(C₃₋₁₀ cycloalkyl)-, —N(heterocyclyl)-, —N(aryl)-, —N(heteroaryl)-, —C(O)—, —C(O)O—, —C(O)NH—, —C(O)N(C₁₋₆ alkyl)-, —C(O)N(C₂₋₆ alkenyl)-, —C(O)N(C₂₋₆ alkynyl)-, —C(O)N(C₁₋₆ haloalkyl)-, —C(O)N(C₃₋₁₀ cycloalkyl)-, —C(O)N(heterocyclyl)-, —C(O)N(aryl)-, —C(O)N(heteroaryl)-, —NHC(O)—, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five hydroxy, halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl.

In certain embodiments, provided is a compound of Formula I:

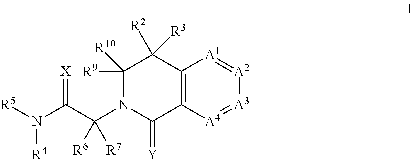

or a pharmaceutically acceptable salt, isotopically enriched analog, stereoisomer, mixture of stereoisomers, or prodrug thereof, wherein:
X is O or S;
Y is O or S;
A¹, A², A³, and A⁴ are each independently N, CH, or CR¹; provided at least one of A¹, A², A³, and A⁴ is CR¹;
each R¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —C(O)R¹¹, —C(O)OR¹¹, —S(O)₀₋₂R¹¹, —NR¹¹S(O)₀₋₂—R¹¹, —S(O)₀₋₂N(R¹¹)₂, —NR¹¹S(O)₀₋₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆alkenyl, C₂₋₆ alkynyl, C₃₋₁₀ cycloalkyl, heterocyclyl, aryl, or heteroaryl is independently optionally substituted with one to eight Z¹; or
any two adjacent R¹ together with the atoms to which they are attached form a cycloalkyl, heterocyclyl, aryl or heteroaryl ring; wherein the cycloalkyl, heterocyclyl, aryl, or heteroaryl is independently optionally substituted with one to eight Z¹;
R² is C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, —NO₂, —SF₅, —OR¹¹, —N(R¹¹)₂, —C(O)R¹², —C(O)OR¹¹, —S(O)₀₋₂—R¹¹, —NR¹¹S(O)₀₋₂—R¹¹, —NR¹¹S(O)₀₋₂N(R¹¹)₂, —NR¹¹C(O)N(R¹¹)₂, —NR¹¹C(O)OR¹¹, —NR¹¹C(O)R¹¹, —OC(O)R¹¹, —OC(O)N(R¹¹)₂, —C(O)N(R¹¹)₂, halo, or cyano; wherein the C₁₋₆ alkyl, C₂₋₆ alkenyl, C₂₋₆ alkynyl, C₁₋₆haloalkyl, C₃₋₁₀ cycloalkyl, heterocyclyl, aryl, or heteroaryl is optionally substituted with one to eight Z¹;
R³ is hydrogen, halo, cyano, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is optionally substituted with one to eight Z¹; or
R² and R³ together form a C₃₋₁₀ cycloalkyl or heterocyclyl ring; wherein the C₃₋₁₀ cycloalkyl or heterocyclyl is optionally substituted with one to eight Z¹;
R⁴ is hydrogen, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is optionally substituted with one to eight Z¹; or
R⁵ is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is optionally substituted with one to eight Z¹; or
R⁴ and R⁵ together form a heterocyclyl or heteroaryl ring optionally substituted with one to eight Z¹;
R⁶ is hydrogen, halo, cyano, C₁₋₆ alkyl, C₂₋₆ alkenyl, C₂₋₆ alkynyl, C₁₋₆ haloalkyl, C₂₋₆ heteroalkyl, C₃₋₁₀ cycloalkyl, or heterocyclyl; wherein the C₁₋₆ alkyl, C₂₋₆ alkenyl, C₂₋₆ alkynyl, C₁₋₆ haloalkyl, C₂₋₆ heteroalkyl, C₃₋₁₀ cycloalkyl, or heterocyclyl may further be optionally substituted with one to five Z^{1b};
R⁷ is hydrogen, halo, cyano, hydroxy, C₁₋₆ alkyl, C₂₋₆ alkenyl, C₂₋₆ alkynyl, C₁₋₆ haloalkyl, C₂₋₆ heteroalkyl, C₃₋₁₀ cycloalkyl, or heterocyclyl; wherein the C₁₋₆ alkyl, C₂₋₆ alkenyl, C₂₋₆ alkynyl, C₁₋₆ haloalkyl, C₂₋₆ heteroalkyl, C₃₋₁₀ cycloalkyl, or heterocyclyl, or may further be optionally substituted with one to five Z^{1b};
or R⁶ and R⁷ join to form a C₃₋₁₀ cycloalkyl or heterocyclyl ring; wherein the C₃₋₁₀ cycloalkyl or heterocyclyl ring may further be optionally substituted with one to five Z^{1b};
R⁹ and R¹⁰ are each independently hydrogen, halo, cyano, C₁₋₆ alkyl, or C₁₋₆ haloalkyl, wherein each C₁₋₆ alkyl or C₁₋₆ haloalkyl is independently optionally substituted with one to five Z¹; or
R⁹ and R¹⁰ together form a C₃₋₁₀ cycloalkyl or heterocyclyl ring; wherein the C₃₋₁₀ cycloalkyl or heterocyclyl is optionally substituted with one to eight Z¹;
each Z¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —C(O)R¹¹, —C(O)OR¹¹, —S(O)₀₋₂R¹¹, —NR¹¹S(O)₀₋₂—R¹¹, —S(O)₀₋₂N(R¹¹)₂, —NR¹¹S(O)₀₋₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each R¹¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹¹ is independently optionally substituted with one to five Z^{1a};
R¹² is C₁₋₆ alkyl, C₂₋₆ alkenyl, C₂₋₆ alkynyl, or C₁₋₆ haloalkyl; wherein each C₁₋₆ alkyl, C₂₋₆ alkenyl, C₂₋₆ alkynyl, or C₁₋₆ haloalkyl of R¹² is independently optionally substituted with one to five Z^{1a};
each Z^{1a} is independently hydroxy, halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, —N(R¹³)₂, —OR¹³, —C(O)R¹³, —C(O)OR¹³, —S(O)₀₋₂R¹³, —NR¹³S(O)₀₋₂—R¹³, —S(O)₀₋₂N(R¹³)₂, —NR¹³S(O)₀₋₂N(R¹³)₂, —NR¹³C(O)N(R¹³)₂, —C(O)N(R¹³)₂, —NR¹³C(O)R¹³, —OC(O)N(R³)₂, or —NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹³ is independently optionally substituted with one to five Z^{1b};
each Z^{1b} is independently halo, cyano, hydroxy, —SH, —NH₂, —NO₂, —SF₅, C₁₋₆ alkyl, C₂₋₆alkenyl, C₂₋₆ alkynyl, C₁₋₆ haloalkyl, C₃₋₁₀ cycloalkyl, heterocyclyl, aryl, heteroaryl, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, —N(C₁₋₆ alkyl)-, —N(C₂₋₆ alkenyl)-, —N(C₂₋₆ alkynyl)-, —N(C₁₋₆ haloalkyl)-, —N(C₃₋₁₀ cycloalkyl)-, —N(heterocyclyl)-, —N(aryl)-, —N(heteroaryl)-, —C(O)—, —C(O)O—, —C(O)NH—, —C(O)N(C₁₋₆ alkyl)-, —C(O)N(C₂₋₆ alkenyl)-, —C(O)N(C₂₋₆ alkynyl)-, —C(O)N(C₁₋₆ haloalkyl)-, —C(O)N(C₃₋₁₀ cycloalkyl)-, —C(O)N(heterocyclyl)-, —C(O)N(aryl)-, —C(O)N(heteroaryl)-, —NHC(O)—, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five hydroxy, halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl.

In certain embodiments. X is O. In certain embodiments, Y is O. In certain embodiments, X is S. In certain embodiments, Y is S. In certain embodiments, X and Y are O. In certain embodiments, X is O and Y is S. In certain embodiments, X is S and Y is O. In certain embodiments, X and Y are S.

In certain embodiments, provided is a compound of Formula IA:

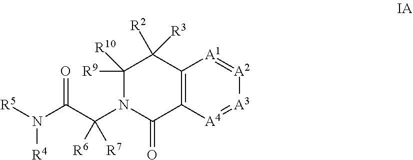

or a pharmaceutically acceptable salt, isotopically enriched analog, stereoisomer, mixture of stereoisomers, or prodrug thereof, wherein:
A¹, A², A³, and A⁴ are each independently N, CH, or CR¹; provided at least one of A¹, A², A³, and A⁴ is CR¹;
each R¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —C(O)R¹¹, —C(O)OR¹¹, —S(O)₀₋₂R¹¹, —NR¹¹S(O)₀₋₂—R¹¹, —S(O)₀₋₂N(R¹¹)₂, —NR¹¹S(O)₀₋₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹; or
any two adjacent R¹ together with the atoms to which they are attached form a cycloalkyl, heterocyclyl, aryl or heteroaryl ring; wherein the cycloalkyl, heterocyclyl, aryl, or heteroaryl is independently optionally substituted with one to eight Z¹;
R² is C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, —NO₂, —SF₅, —OR¹¹, —N(R¹¹)₂, —C(O)R¹², —C(O)OR¹¹, —S(O)₀₋₂—R¹¹, —NR¹S(O)₀₋₂—R¹¹, —NR¹¹S(O)₀₋₂N(R¹¹)₂, —NR¹¹C(O)N(R¹¹)₂, —NR¹¹C(O)OR¹¹, —NR¹¹C(O)R¹¹, —OC(O)R¹¹, —OC(O)N(R¹¹)₂, —C(O)N(R¹¹)₂, halo, or cyano; wherein the C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is optionally substituted with one to eight Z¹;
R³ is hydrogen, halo, cyano, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is optionally substituted with one to eight Z¹; or
R² and R³ together form a C₃₋₁₀ cycloalkyl or heterocyclyl ring; wherein the C₃₋₁₀ cycloalkyl or heterocyclyl is optionally substituted with one to eight Z¹;
R⁴ is hydrogen, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is optionally substituted with one to eight Z¹; or
R⁵ is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is optionally substituted with one to eight Z¹; or
R⁴ and R⁵ together form a heterocyclyl or heteroaryl ring optionally substituted with one to eight Z¹;
R⁶ is hydrogen, halo, cyano, C₁₋₆ alkyl, C₂₋₆ alkenyl, C₂₋₆ alkynyl, C₁₋₆ haloalkyl, C₂₋₆ heteroalkyl, C₃₋₁₀ cycloalkyl, or heterocyclyl; wherein the C₁₋₆ alkyl, C₂₋₆ alkenyl, C₂₋₆ alkynyl, C₁₋₆ haloalkyl, C₂₋₆ heteroalkyl, C₃₋₁₀ cycloalkyl, or heterocyclyl may further be optionally substituted with one to five Z^{1b};
R⁷ is hydrogen, halo, cyano, hydroxy, C₁₋₆ alkyl, C₂₋₆ alkenyl, C₂₋₆ alkynyl, C₁₋₆ haloalkyl, C₂₋₆ heteroalkyl, C₃₋₁₀ cycloalkyl, or heterocyclyl; wherein the C₁₋₆ alkyl, C₂₋₆ alkenyl, C₂₋₆ alkynyl, C₁₋₆ haloalkyl, C₂₋₆ heteroalkyl, C₃₋₁₀ cycloalkyl, or heterocyclyl, or may further be optionally substituted with one to five Z^{1b};
or R⁶ and R⁷ join to form a C₃₋₁₀ cycloalkyl or heterocyclyl ring; wherein the C₃₋₁₀ cycloalkyl or heterocyclyl ring may further be optionally substituted with one to five Z^{1b};
R⁹ and R¹⁰ are each independently hydrogen, halo, cyano, C₁₋₆ alkyl, or C₁₋₆ haloalkyl, wherein each C₁₋₆ alkyl or C₁₋₆ haloalkyl is independently optionally substituted with one to five Z¹; or
R⁹ and R¹⁰ together form a C₃₋₁₀ cycloalkyl or heterocyclyl ring; wherein the C₃₋₁₀ cycloalkyl or heterocyclyl is optionally substituted with one to eight Z¹;
each Z¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —C(O)R¹¹, —C(O)OR¹¹, —S(O)₀₋₂R¹¹, —NR¹¹S(O)₀₋₂—R¹¹, —S(O)₀₋₂N(R¹¹)₂, —NR¹¹S(O)₀₋₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each R¹¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹¹ is independently optionally substituted with one to five Z^{1a};
R¹² is C₁₋₆alkyl, C₂₋₆ alkenyl, C₂₋₆ alkynyl, or C₁₋₆ haloalkyl; wherein each C₁₋₆ alkyl, C₂₋₆ alkenyl, C₂₋₆ alkynyl, or C₁₋₆ haloalkyl of R¹² is independently optionally substituted with one to five Z^{1a};
each Z^{1a} is independently hydroxy, halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, —N(R¹³)₂, —OR¹³, —C(O)R¹³, —C(O)OR¹³, —S(O)₀₋₂R¹³, —NR¹³S(O)₀₋₂—R¹³, —S(O)₀₋₂N(R¹³)₂, —NR¹³S(O)₀₋₂N(R¹³)₂, —NR¹³C(O)N(R¹³)₂, —C(O)N(R¹³)₂, —NR¹³C(O)R¹³, —OC(O)N(R¹³)₂, or —NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹³ is independently optionally substituted with one to five Z^{1b};
each Z^{1b} is independently halo, cyano, hydroxy, —SH, —NH₂, —NO₂, —SF₅, C₁₋₆ alkyl, C₂₋₆alkenyl, C₂₋₆ alkynyl, C₁₋₆ haloalkyl, C₃₋₁₀ cycloalkyl, heterocyclyl, aryl, heteroaryl, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, —N(C₁₋₆ alkyl)-, —N(C₂₋₆ alkenyl)-, —N(C₂₋₆ alkynyl)-, —N(C₁₋₆ haloalkyl)-, —N(C₃₋₁₀ cycloalkyl)-, —N(heterocyclyl)-, —N(aryl)-, —N(heteroaryl)-, —C(O)—, —C(O)O—, —C(O)NH—, —C(O)N(C₁₋₆ alkyl)-, —C(O)N(C₂₋₆ alkenyl)-, —C(O)N(C₂₋₆ alkynyl)-, —C(O)N(C₁₋₆ haloalkyl)-, —C(O)N(C₃₋₁₀ cycloalkyl)-, —C(O)N(heterocyclyl)-, —C(O)N(aryl)-, —C(O)N(heteroaryl)-, —NHC(O)—, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five hydroxy, halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl.

In certain embodiments, A², A³ and A⁴ are each independently N, CH, or CR¹; and A¹ is CR¹.

In certain embodiments, A¹, A³ and A⁴ are each independently N, CH, or CR¹; and A² is CR¹.

In certain embodiments, A¹, A², and A⁴ are each independently N, CH, or CR¹; and A³ is CR¹.

In certain embodiments, A¹, A², and A³ are each independently N, CH, or CR¹; and A⁴ is CR¹.

In certain embodiments, at least one of A¹, A², A³, and A⁴ is N.

In certain embodiments, A¹, A², A³, and A⁴ are each independently CH or CR¹.

In certain embodiments, A², A³, and A⁴ are each independently CH or CR¹; and A¹ is CR¹.

In certain embodiments, A¹, A³, and A⁴ are each independently CH or CR¹; and A² is CR¹.

In certain embodiments, A¹, A², and A⁴ are each independently CH or CR¹; and A³ is CR¹.

In certain embodiments, A¹, A², and A³ are each independently CH or CR¹; and A⁴ is CR¹.

In certain embodiments, provided is a compound represented by Formula IB:

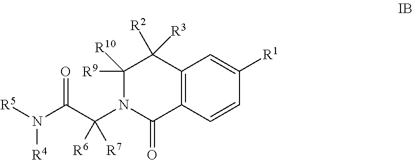

wherein R¹, R², R³, R⁴, R⁵, R⁶, R⁷, R⁹, and R¹⁰ are each independently as defined herein.

In certain embodiments, R⁴ is hydrogen or C₁₋₆ alkyl. In certain embodiments, R⁴ is hydrogen or methyl. In certain embodiments, R⁴ is hydrogen. In certain embodiments, R⁴ is C₁₋₆ alkyl. In certain embodiments, R⁴ is methyl.

In certain embodiments, R⁵ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is optionally substituted with one to eight Z¹; or R⁴ and R⁵ together form a heterocyclyl or heteroaryl ring optionally substituted with one to eight Z¹.

In certain embodiments. R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted with one to five Z¹. In certain embodiments, R⁴ is hydrogen; and R⁵ is C₁₋₆ alkyl optionally substituted with one to eight Z¹. In certain embodiments, R⁴ is hydrogen; and R⁵ is (1-(2,2-difluoroethyl)cyclobutyl)methyl, oxetan-3-ylmethyl, oxazol-2-ylmethyl, (1-methyl-1H-imidazol-2-yl)methyl, 2-(1H-imidazol-1-yl)ethyl, pyridin-4-ylmethyl, (1-methyl-1H-pyrazol-4-yl)methyl, (1-methyl-1H-pyrazol-5-yl)methyl, 2-morpholinoethyl, 2-(4-fluorophenyl)-2-hydroxyethyl, 3,3,3-trifluoropropyl, 2-cyanopropan-2-yl, 2-(methylsulfonamido)ethyl, (2-(trifluoromethyl)pyridin-3-yl)methyl, cyclobutylmethyl, 3-hydroxy-3-methylbutyl or 2-hydroxy-2-methyl-propyl. In certain embodiments, R⁴ is hydrogen; and R⁵ is 3-hydroxy-3-methylbutyl or 2-hydroxy-2-methyl-propyl.

In certain embodiments, R⁵ is (1-(2,2-difluoroethyl)cyclobutyl)methyl, (1-methyl-1H-imidazol-2-yl)methyl, (1-methyl-1H-pyrazol-4-yl)methyl, (1-methyl-1H-pyrazol-5-yl)methyl, (1R,2R,4S)-7-oxabicyclo[2.2.1]heptan-2-yl, (1S,2R,4R)-7-oxabicyclo[2.2.1]heptan-2-yl, (2-(trifluoromethyl)pyridin-3-yl)methyl, [1,2,4]triazolo[1,5-a]pyridin-2-yl, [1,2,4]triazolo[4,3-a]pyridin-6-yl, [1,2,4]triazolo[1,5-a]pyrazin-2-yl, 7-chloro-[1,2,4]triazolo[1,5-a]pyridin-2-yl, 7-(trifluoromethyl)-[1,2,4]triazolo[1,5-a]pyridin-2-yl, 6-chloro-[1,2,4]triazolo[1,5-a]pyridin-2-yl, 6-fluoro-[1,2,4]triazolo[1,5-a]pyridin-2-yl, 6-methoxy-[1,2,4]triazolo[1,5-a]pyridin-2-yl, 1-(2-hydroxy-2-methylpropyl)cyclopropyl, 1-(2-methoxyethyl)-1H-pyrazol-4-yl, 1-(2-methoxyethyl)-3-piperidyl, 1-(6-chloropyridazin-3-yl)piperidin-4-yl, 1-(hydroxymethyl)cyclopropyl, 1-(methoxycarbonyl)piperidin-3-yl, 1,1-dioxidothietan-3-yl, 1,3,5-triazin-2-yl, 1,3-dimethyl-1H-pyrazol-5-yl, 1,6-naphthyridin-2-yl, 1,7-naphthyridin-6-yl, 1,8-naphthyridin-2-yl, 1-bicyclo[2.2.2]octanyl, 1-cyclobutylpiperidin-3-yl, 1-cyclopropylpiperidin-3-yl, 1-ethyl-6-oxo-3-piperidyl, 1-ethylpiperidin-3-yl, 1H-benzo[d][1,2,3]triazol-5-yl, 1H-benzo[d]imidazol-2-yl, 1H-benzo[d]imidazol-6-yl, 1H-indazol-3-yl, 1H-indazol-5-yl, 1H-indazol-6-yl, 1H-indol-6-yl, 1H-pyrrolo[2,3-b]pyridin-5-yl, 1-methyl-1H-1,2,4-triazol-5-yl, 1-methyl-1H-benzo[d]imidazol-5-yl, 1-methyl-1H-indazol-5-yl, 1-methyl-1H-pyrazolo[4,3-b]pyridin-5-yl, 1-methyl-2-oxabicyclo[2.1.1]hexan-4-yl, 1-methyl-2-oxo-4-piperidyl, 1-methyl-5-oxo-pyrrolidin-3-yl, 1-methyl-6-oxo-3-piperidyl, 1-methyl-6-oxo-3-pyridyl, 1-phenyl-1H-pyrazol-5-yl, 1-phenylcyclopropyl, 2-(1H-imidazol-1-yl)ethyl, 2-(4-fluorophenyl)-2-hydroxyethyl, 2-(difluoromethoxy)phenyl, 2-(methylsulfonamido)ethyl, 2-(methylsulfonyl)ethyl, 2,2-difluorobenzo[d][1,3]dioxol-5-yl, 2,3-dihydro-1H-inden-2-yl, 2,3-dihydrobenzofuran-5-yl, 2,6-dimethylpyrimidin-4-yl, 2-chloro-4-(methylsulfonyl)phenyl, 2-cyanopropan-2-yl, 2-cyclopropyltetrahydropyran-4-yl, 2-hydroxy-2-methyl-propyl, 2-methyl-2H-pyrazolo[4,3-b]pyridin-5-yl, 2-methylbenzo[d]thiazol-6-yl, 2-morpholinoethyl, 2-oxabicyclo[2.2.2]octan-4-yl, 2-oxaspiro[3.3]heptan-6-yl, 3-(1-hydroxy-1-methyl-ethyl)-1-bicyclo[1.1.1]pentanyl, 3-(2-methylthiazol-4-yl)phenyl, 3-(difluoromethoxy)cyclobutyl, 3-(difluoromethyl)cyclobutyl, 3-(hydroxymethyl)cyclobutyl, 3-(trifluoromethyl)-1-bicyclo[1.1.1]pentanyl, 3-(trifluoromethyl)cyclobutyl, 3,3,3-trifluoropropyl, 3,3-difluorocyclobutyl, 3,4-dimethylisoxazol-5-yl, 3,5-difluoro-2-pyridyl, 3-cyano-1-bicyclo[1.1.1]pentanyl, 3-cyanocyclobutyl, 3-cyclopropyl-1H-pyrazol-5-yl, 3-cyclopropyl-1-methyl-1H-pyrazol-5-yl, 3-fluoro-5-(1H-pyrazol-1-yl)pyridin-2-yl, 3-fluoro-5-(trifluoromethyl)pyridin-2-yl, 3-fluoro-5-formylpyridin-2-yl, 3-fluoropyridin-4-yl, 3-hydroxy-3-(trifluoromethyl)cyclobutyl, 3-hydroxy-3-methylbutyl, 3-hydroxy-3-methylcyclobutyl, 3-hydroxycyclohexyl, 3-methyl-1-phenyl-1H-pyrazol-5-yl, 3-methylcyclobutyl, 4-(1H-tetrazol-5-yl)phenyl, 4-(2-methylthiazol-4-yl)pyrimidin-2-yl, 4,4-difluorocyclohexyl, 4,5,6,7-tetrahydro-1H-indazol-6-yl, 4,5,6,7-tetrahydropyrazolo[1,5-a]pyridin-5-yl, 4,5-dimethylpyrimidin-2-yl, 4,6-dimethylpyridin-2-yl, 4-cyanopyrimidin-2-yl, 4-hydroxy-1-bicyclo[2.2.2]octanyl, 4-methylpyridin-2-yl, 5-(difluoromethoxy)-2-pyridyl, 5-(difluoromethyl)pyridin-2-yl, 5-(pyridin-2-yl)pyrimidin-2-yl, 5-(trifluoromethyl)pyrimidin-2-yl, 5-(difluoromethoxy)pyrimidin-2-yl, 5,7-dihydrofuro[3,4-d]pyrimidin-2-yl, 5-chloro-3-fluoropyridin-2-yl, 5-chloropyridin-2-yl, 5-chloropyrimidin-2-yl, 5-cyano-3-fluoropyridin-2-yl, 5-cyanobenzo[d]oxazol-2-yl, 5-cyanopyridin-2-yl, 5-cyanopyrimidin-2-yl, 5-cyclopropylpyrimidin-2-yl, 5-cyclobutylpyrimidin-2-yl, 5-ethylpyrimidin-2-yl, 5-fluoro-4-methylpyrimidin-2-yl, 5-cyano-4-methylpyrimidin-2-yl, 5-fluoropyridin-2-yl, 5-fluoropyrimidin-2-yl, 5-fluoropyrimidin-4-yl, 5-iodopyrimidin-2-yl, 5-methoxypyrimidin-2-yl, 5-methyl-2-oxo-1,2-dihydropyridin-3-yl, 5-methylpyrimidin-2-yl, 5-pyrazol-1-ylpyrimidin-2-yl, 5-(tetrahydrofuran-3-yl)pyrimidin-2-yl, 5-(1-methyl-1H-pyrazol-4-yl)pyrimidin-2-yl, 6,7-dihydro-5H-pyrrolo[1,2-b][1,2,4]triazol-2-yl, 5-fluorothiazol-2-yl, 6-chloropyridazin-3-yl, 6-fluorobenzo[d]oxazol-2-yl, 6-cyanobenzo[d]oxazol-2-yl, 6-methylpyrazin-2-yl, 6-methylpyridin-2-yl, 6-oxo-1,6-dihydropyrimidin-2-yl, benzo[d]oxazol-2-yl, benzo[d]oxazol-5-yl, benzo[d]thiazol-5-yl, benzo[d]thiazol-6-yl, cyclobutylmethyl, imidazo[1,2-a]pyrazin-6-yl, imidazo[1,2-a]pyridin-5-yl, imidazo[1,2-a]pyridin-8-yl, imidazo[1,2-b]pyridazin-6-yl, imidazo[1,5-a]pyridin-6-yl, isoquinolin-4-yl, isoquinolin-6-yl, isoquinolin-7-yl, isoquinolin-8-yl, isoxazolo[4,5-b]pyridin-5-yl, isoxazolo[5,4-b]pyridin-6-yl, oxazol-2-ylmethyl, oxazolo[4,5-b]pyridin-2-yl, oxazolo[4,5-c]pyridin-2-yl, oxazolo[5,4-b]pyridin-2-yl, oxazolo[5,4-c]pyridin-2-yl, oxetan-3-ylmethyl, phenyl, pyrazolo[1,5-a]pyrimidin-5-yl, pyridin-4-ylmethyl, pyrimidin-2-yl, quinazolin-2-yl, quinolin-2-yl, quinolin-3-yl, quinolin-5-yl, quinolin-6-yl, spiro[2.3]hexan-5-yl, [1,2,4]triazolo[1,5-a]pyrazin-8-yl, [1,2,4]triazolo[4,3-a]pyrazin-8-yl, [1,3]thiazolo[5,4-d]pyrimidin-5-yl, 1-(1-methylpyrazol-3-yl)pyrrolidin-3-yl, 1-(1-methylpyrazol-4-yl)piperidin-3-yl, 1-(1-methylpyrazol-4-yl)pyrrolidin-3-yl, 1-(2,2,2-trifluoroethyl)-1,2,4-triazol-3-yl, 1-(2,2,2-trifluoroethyl)piperidin-4-yl, 1-(2,2-difluoroethyl)piperidin-4-yl, 1-(3,3,3-trifluoropropyl)piperidin-4-yl, 1-(oxetan-3-yl)piperidin-3-yl, 1-(oxetan-3-yl)pyrrolidin-3-yl, 1,2,4-benzotriazin-3-yl, 1,2-benzothiazol-6-yl, 1,2-benzoxazol-3-yl, 1,5-dimethyl-1,2,4-triazol-3-yl, 1,7-naphthyridin-8-yl, 1-azabicyclo[2.2.2]octan-3-yl, 1-benzylpyrrolidin-3-yl, 1-cyclopropyl-1,2,4-triazol-3-yl, 1-ethyl-1-azaspiro[3.3]heptan-6-yl, 1-ethylpyrrolidin-3-yl, 1-methyl-1,2,4-triazol-3-yl, 1-methyl-2-oxopyrrolidin-3-yl, 1-methyl-6-oxopyridazin-3-yl, 1-methylpiperidin-3-yl, 1-methylpyrazolo[3,4-d]pyrimidin-6-yl, 1-phenyl-1,2,4-triazol-3-yl, 1-propan-2-yl-1,2,4-triazol-3-yl, 1-pyridazin-3-ylpiperidin-4-yl, 1-pyridin-2-ylpiperidin-4-yl, 1-pyridin-3-ylpiperidin-4-yl, 1-pyrimidin-2-ylpiperidin-4-yl, 2-methylimidazo[1,2-b]pyridazin-6-yl, 2-oxopyrrolidin-3-yl, 3-(1H-pyrazol-5-yl)cyclobutyl, 3-(methoxymethyl)cyclobutyl, 3-chloro-5-cyanopyridin-2-yl, 3-cyano-5-fluoropyridin-2-yl, 3-fluoro-5-methylpyridin-2-yl, 3-fluoroimidazo[1,2-a]pyridin-2-yl, 3-fluoropyrazolo[1,5-a]pyridin-2-yl, 3-methoxy-3-methylcyclobutyl, 3-methoxypyridin-2-yl, 3-methylimidazo[1,2-b]pyridazin-6-yl, 3-methylpyrazolo[1,5-a]pyridin-2-yl, 3-phenylcyclobutyl, 3-phenylmethoxycyclobutyl, 4,4-dimethyl-5H-1,3-oxazol-2-yl, 4,5,6,7-tetrahydro-1,3-benzoxazol-2-yl, 4-cyano-1,3-benzoxazol-2-yl, 4-methoxypyrimidin-2-yl, 4-methyl-3-oxopyrazin-2-yl, 4-methyl-4-azaspiro[2.5]octan-7-yl, 4-methyl-5-oxopyrazin-2-yl, 5-(2,2-difluorocyclopropyl)pyrimidin-2-yl, 5-(2,3-dihydrofuran-4-yl)pyrimidin-2-yl, 5-(difluoromethyl)-3-fluoropyridin-2-yl, 5-(methoxymethoxy)pyrimidin-2-yl, 5-(oxetan-3-yl)pyrimidin-2-yl, 5-(oxolan-2-yl)pyrimidin-2-yl, 5-(trifluoromethyl)-1,3-benzoxazol-2-yl, 5,5-dimethyl-4H-1,3-oxazol-2-yl, 5,6,7,8-tetrahydro-[1,2,4]triazolo[1,5-a]pyridin-2-yl, 5,6-dihydrofuro[2,3-d]pyrimidin-2-yl, 5-cyano-3-fluoro-4-methylpyridin-2-yl, 5-cyano-3-fluoro-6-methylpyridin-2-yl, 5-cyano-3-methylpyridin-2-yl, 5-fluoro-2-methoxypyrimidin-4-yl, 5-fluoro-6-methoxypyrimidin-4-yl, 5-methyl-1-phenyl-1,2,4-triazol-3-yl, 5-pyrrolidin-1-ylpyrimidin-2-yl, 6-(difluoromethoxy)pyridin-3-yl, 6-(trifluoromethyl)-1,3-benzoxazol-2-yl, 6,7-dihydro-4H-pyrazolo[5,1-c][1,4]oxazin-2-yl, 6,8-dihydro-5H-pyrano[3,4-d]pyrimidin-2-yl, 6-cyano-4-fluoropyridin-3-yl, 6-cyanopyridin-3-yl, 6-fluoro-1,3-benzoxazol-2-yl, 6-fluoropyrazolo[1,5-a]pyrimidin-5-yl, 6-methoxypyridin-3-yl, 7,8-dihydro-5H-pyrano[4,3-d]pyrimidin-2-yl, 7-methylpyrazolo[1,5-a]pyrimidin-5-yl, 8-chloro-[1,2,4]triazolo[1,5-a]pyridin-2-yl, 1-(ethoxycarbonyl)piperidin-4-yl, imidazo[1,2-a]pyrazin-8-yl, imidazo[1,2-a]pyridin-2-yl, imidazo[1,2-a]pyrimidin-7-yl, imidazo[1,2-c]pyrimidin-5-yl, pyrazin-2-yl, pyrazolo[1,5-a]pyridin-2-yl, pyridazin-4-yl, 1-(tert-butoxycarbonyl)-1-azaspiro[3.3]heptan-6-yl, or 6-oxo-1,6-dihydropyridazin-3-yl.

In certain embodiments. R⁵ is (1-(2,2-difluoroethyl)cyclobutyl)methyl, (1-methyl-1H-imidazol-2-yl)methyl, (1-methyl-1H-pyrazol-4-yl)methyl, (1-methyl-1H-pyrazol-5-yl)methyl, (1R,2R,4S)-7-oxabicyclo[2.2.1]heptan-2-yl, (1S,2R,4R)-7-oxabicyclo[2.2.1]heptan-2-yl, (2-(trifluoromethyl)pyridin-3-yl)methyl, [1,2,4]triazolo[1,5-a]pyridin-2-yl, [1,2,4]triazolo[4,3-a]pyridin-6-yl, [1,2,4]triazolo[1,5-a]pyrazin-2-yl, 7-chloro-[1,2,4]triazolo[1,5-a]pyridin-2-yl, 7-(trifluoromethyl)-[1,2,4]triazolo[1,5-a]pyridin-2-yl, 6-chloro-[1,2,4]triazolo[1,5-a]pyridin-2-yl, 6-fluoro-[1,2,4]triazolo[1,5-a]pyridin-2-yl, 6-methoxy-[1,2,4]triazolo[1,5-a]pyridin-2-yl, 1-(2-hydroxy-2-methylpropyl)cyclopropyl, 1-(2-methoxyethyl)-1H-pyrazol-4-yl, 1-(2-methoxyethyl)-3-piperidyl, 1-(6-chloropyridazin-3-yl)piperidin-4-yl, 1-(hydroxymethyl)cyclopropyl, 1-(methoxycarbonyl)piperidin-3-yl, 1,1-dioxidothietan-3-yl, 1,3,5-triazin-2-yl, 1,3-dimethyl-1H-pyrazol-5-yl, 1,6-naphthyridin-2-yl, 1,7-naphthyridin-6-yl, 1,8-naphthyridin-2-yl, 1-bicyclo[2.2.2]octanyl, 1-cyclobutylpiperidin-3-yl, 1-cyclopropylpiperidin-3-yl, 1-ethyl-6-oxo-3-piperidyl, 1-ethylpiperidin-3-yl, 1H-benzo[d][1,2,3]triazol-5-yl, 1H-benzo[d]imidazol-2-yl, 1H-benzo[d]imidazol-6-yl, 1H-indazol-3-yl, 1H-indazol-5-yl, 1H-indazol-6-yl, 1H-indol-6-yl, 1H-pyrrolo[2,3-b]pyridin-5-yl, 1-methyl-1H-1,2,4-triazol-5-yl, 1-methyl-1H-benzo[d]imidazol-5-yl, 1-methyl-1H-indazol-5-yl, 1-methyl-1H-pyrazolo[4,3-b]pyridin-5-yl, 1-methyl-2-oxabicyclo[2.1.1]hexan-4-yl, 1-methyl-2-oxo-4-piperidyl, 1-methyl-5-oxo-pyrrolidin-3-yl, 1-methyl-6-oxo-3-piperidyl, 1-methyl-6-oxo-3-pyridyl, 1-phenyl-1H-pyrazol-5-yl, 1-phenylcyclopropyl, 2-(1H-imidazol-1-yl)ethyl, 2-(4-fluorophenyl)-2-hydroxyethyl, 2-(difluoromethoxy)phenyl, 2-(methylsulfonamido)ethyl, 2-(methylsulfonyl)ethyl, 2,2-difluorobenzo[d][1,3]dioxol-5-yl, 2,3-dihydro-1H-inden-2-yl, 2,3-dihydrobenzofuran-5-yl, 2,6-dimethylpyrimidin-4-yl, 2-chloro-4-(methylsulfonyl)phenyl, 2-cyanopropan-2-yl, 2-cyclopropyltetrahydropyran-4-yl, 2-hydroxy-2-methyl-propyl, 2-methyl-2H-pyrazolo[4,3-b]pyridin-5-yl, 2-methylbenzo[d]thiazol-6-yl, 2-morpholinoethyl, 2-oxabicyclo[2.2.2]octan-4-yl, 2-oxaspiro[3.3]heptan-6-yl, 3-(1-hydroxy-1-methyl-ethyl)-1-bicyclo[1.1.1]pentanyl, 3-(2-methylthiazol-4-yl)phenyl, 3-(difluoromethoxy)cyclobutyl, 3-(difluoromethyl)cyclobutyl, 3-(hydroxymethyl)cyclobutyl, 3-(trifluoromethyl)-1-bicyclo[1.1.1]pentanyl, 3-(trifluoromethyl)cyclobutyl, 3,3,3-trifluoropropyl, 3,3-difluorocyclobutyl, 3,4-dimethylisoxazol-5-yl, 3,5-difluoro-2-pyridyl, 3-cyano-1-bicyclo[1.1.1]pentanyl, 3-cyanocyclobutyl, 3-cyclopropyl-1H-pyrazol-5-yl, 3-cyclopropyl-1-methyl-1H-pyrazol-5-yl, 3-fluoro-5-(1H-pyrazol-1-yl)pyridin-2-yl, 3-fluoro-5-(trifluoromethyl)pyridin-2-yl, 3-fluoro-5-formylpyridin-2-yl, 3-fluoropyridin-4-yl, 3-hydroxy-3-(trifluoromethyl)cyclobutyl, 3-hydroxy-3-methylbutyl, 3-hydroxy-3-methylcyclobutyl, 3-hydroxycyclohexyl, 3-methyl-1-phenyl-1H-pyrazol-5-yl, 3-methylcyclobutyl, 4-(1H-tetrazol-5-yl)phenyl, 4-(2-methylthiazol-4-yl)pyrimidin-2-yl, 4,4-difluorocyclohexyl, 4,5,6,7-tetrahydro-1H-indazol-6-yl, 4,5,6,7-tetrahydropyrazolo[1,5-a]pyridin-5-yl, 4,5-dimethylpyrimidin-2-yl, 4,6-dimethylpyridin-2-yl, 4-cyanopyrimidin-2-yl, 4-hydroxy-1-bicyclo[2.2.2]octanyl, 4-methylpyridin-2-yl, 5-(difluoromethoxy)-2-pyridyl, 5-(difluoromethyl)pyridin-2-yl, 5-(pyridin-2-yl)pyrimidin-2-yl, 5-(trifluoromethyl)pyrimidin-2-yl, 5-(difluoromethoxy)pyrimidin-2-yl, 5,7-dihydrofuro[3,4-d]pyrimidin-2-yl, 5-chloro-3-fluoropyridin-2-yl, 5-chloropyridin-2-yl, 5-chloropyrimidin-2-yl, 5-cyano-3-fluoropyridin-2-yl, 5-cyanobenzo[d]oxazol-2-yl, 5-cyanopyridin-2-yl, 5-cyanopyrimidin-2-yl, 5-cyclopropylpyrimidin-2-yl, 5-cyclobutylpyrimidin-2-yl, 5-ethylpyrimidin-2-yl, 5-fluoro-4-methylpyrimidin-2-yl, 5-cyano-4-methylpyrimidin-2-yl, 5-fluoropyridin-2-yl, 5-fluoropyrimidin-2-yl, 5-fluoropyrimidin-4-yl, 5-iodopyrimidin-2-yl, 5-methoxypyrimidin-2-yl, 5-methyl-2-oxo-1,2-dihydropyridin-3-yl, 5-methylpyrimidin-2-yl, 5-pyrazol-1-ylpyrimidin-2-yl, 5-(tetrahydrofuran-3-yl)pyrimidin-2-yl, 5-(1-methyl-1H-pyrazol-4-yl)pyrimidin-2-yl, 6,7-dihydro-5H-pyrrolo[1,2-b][1,2,4]triazol-2-yl, 5-fluorothiazol-2-yl, 6-chloropyridazin-3-yl, 6-fluorobenzo[d]oxazol-2-yl, 6-cyanobenzo[d]oxazol-2-yl, 6-methylpyrazin-2-yl, 6-methylpyridin-2-yl, 6-oxo-1,6-dihydropyrimidin-2-yl, benzo[d]oxazol-2-yl, benzo[d]oxazol-5-yl, benzo[d]thiazol-5-yl, benzo[d]thiazol-6-yl, cyclobutylmethyl, imidazo[1,2-a]pyrazin-6-yl, imidazo[1,2-a]pyridin-5-yl, imidazo[1,2-a]pyridin-8-yl, imidazo[1,2-b]pyridazin-6-yl, imidazo[1,5-a]pyridin-6-yl, isoquinolin-4-yl, isoquinolin-6-yl, isoquinolin-7-yl, isoquinolin-8-yl, isoxazolo[4,5-b]pyridin-5-yl, isoxazolo[5,4-b]pyridin-6-yl, oxazol-2-ylmethyl, oxazolo[4,5-b]pyridin-2-yl, oxazolo[4,5-c]pyridin-2-yl, oxazolo[5,4-b]pyridin-2-yl, oxazolo[5,4-c]pyridin-2-yl, oxetan-3-ylmethyl, phenyl, pyrazolo[1,5-a]pyrimidin-5-yl, pyridin-4-ylmethyl, pyrimidin-2-yl, quinazolin-2-yl, quinolin-2-yl, quinolin-3-yl, quinolin-5-yl, quinolin-6-yl, or spiro[2.3]hexan-5-yl.

In certain embodiments, R⁵ is (1-(2,2-difluoroethyl)cyclobutyl)methyl, (1-methyl-1H-imidazol-2-yl)methyl, (1-methyl-1H-pyrazol-4-yl)methyl, (1-methyl-1H-pyrazol-5-yl)methyl, (1R,2R,4S)-7-oxabicyclo[2.2.1]heptan-2-yl, (1S,2R,4R)-7-oxabicyclo[2.2.1]heptan-2-yl, (2-(trifluoromethyl)pyridin-3-yl)methyl, [1,2,4]triazolo[1,5-a]pyridin-2-yl, [1,2,4]triazolo[4,3-a]pyridin-6-yl, 1-(2-hydroxy-2-methylpropyl)cyclopropyl, 1-(2-methoxyethyl)-1H-pyrazol-4-yl, 1-(2-methoxyethyl)-3-piperidyl, 1-(6-chloropyridazin-3-yl)piperidin-4-yl, 1-(hydroxymethyl)cyclopropyl, 1-(methoxycarbonyl)piperidin-3-yl, 1,1-dioxidothietan-3-yl, 1,3,5-triazin-2-yl, 1,3-dimethyl-1H-pyrazol-5-yl, 1,6-naphthyridin-2-yl, 1,7-naphthyridin-6-yl, 1,8-naphthyridin-2-yl, 1-bicyclo[2.2.2]octanyl, 1-cyclobutylpiperidin-3-yl, 1-ethyl-6-oxo-3-piperidyl. 1-ethylpiperidin-3-yl, 1H-benzo[d][1,2,3]triazol-5-yl, 1H-benzo[d]imidazol-2-yl, 1H-benzo[d]imidazol-6-yl, 1H-indazol-3-yl, 1H-indazol-5-yl, 1H-indazol-6-yl, 1H-indol-6-yl, 1H-pyrrolo[2,3-b]pyridin-5-yl, 1-methyl-1H-1,2,4-triazol-5-yl, 1-methyl-1H-benzo[d]imidazol-5-yl, 1-methyl-1H-indazol-5-yl, 1-methyl-1H-pyrazolo[4,3-b]pyridin-5-yl, 1-methyl-2-oxabicyclo[2.1.1]hexan-4-yl, 1-methyl-2-oxo-4-piperidyl, 1-methyl-5-oxo-pyrrolidin-3-yl, 1-methyl-6-oxo-3-piperidyl, 1-methyl-6-oxo-3-pyridyl, 1-phenyl-1H-pyrazol-5-yl, 1-phenylcyclopropyl, 2-(1H-imidazol-1-yl)ethyl, 2-(4-fluorophenyl)-2-hydroxyethyl, 2-(difluoromethoxy)phenyl, 2-(methylsulfonamido)ethyl, 2-(methylsulfonyl)ethyl, 2,2-difluorobenzo[d][1,3]dioxol-5-yl, 2,3-dihydro-1H-inden-2-yl, 2,3-dihydrobenzofuran-5-yl, 2,6-dimethylpyrimidin-4-yl, 2-chloro-4-(methylsulfonyl)phenyl, 2-cyanopropan-2-yl, 2-cyclopropyltetrahydropyran-4-yl, 2-hydroxy-2-methyl-propyl, 2-methyl-2H-pyrazolo[4,3-b]pyridin-5-yl, 2-methylbenzo[d]thiazol-6-yl, 2-morpholinoethyl, 2-oxabicyclo[2.2.2]octan-4-yl, 2-oxaspiro[3.3]heptan-6-yl, 3-(1-hydroxy-1-methyl-ethyl)-1-bicyclo[1.1.1]pentanyl, 3-(2-methylthiazol-4-yl)phenyl, 3-(difluoromethoxy)cyclobutyl, 3-(difluoromethyl)cyclobutyl, 3-(hydroxymethyl)cyclobutyl, 3-(trifluoromethyl)-1-bicyclo[1.1.1]pentanyl, 3-(trifluoromethyl)cyclobutyl, 3,3,3-trifluoropropyl, 3,3-difluorocyclobutyl, 3,4-dimethylisoxazol-5-yl, 3,5-difluoro-2-pyridyl, 3-cyano-1-bicyclo[1.1.1]pentanyl, 3-cyanocyclobutyl, 3-cyclopropyl-1H-pyrazol-5-yl, 3-cyclopropyl-1-methyl-1H-pyrazol-5-yl, 3-fluoro-5-(1H-pyrazol-1-yl)pyridin-2-yl, 3-fluoro-5-(trifluoromethyl)pyridin-2-yl, 3-fluoro-5-formylpyridin-2-yl, 3-fluoropyridin-4-yl, 3-hydroxy-3-(trifluoromethyl)cyclobutyl, 3-hydroxy-3-methylbutyl, 3-hydroxy-3-methylcyclobutyl, 3-hydroxycyclohexyl, 3-methyl-1-phenyl-1H-pyrazol-5-yl, 3-methylcyclobutyl, 4-(1H-tetrazol-5-yl)phenyl, 4-(2-methylthiazol-4-yl)pyrimidin-2-yl, 4,4-difluorocyclohexyl, 4,5,6,7-tetrahydro-1H-indazol-6-yl, 4,5,6,7-tetrahydropyrazolo[1,5-a]pyridin-5-yl, 4,5-dimethylpyrimidin-2-yl, 4,6-dimethylpyridin-2-yl, 4-cyanopyrimidin-2-yl, 4-hydroxy-1-bicyclo[2.2.2]octanyl, 4-methylpyridin-2-yl, 5-(difluoromethoxy)-2-pyridyl, 5-(difluoromethyl)pyridin-2-yl, 5-(pyridin-2-yl)pyrimidin-2-yl, 5-(trifluoromethyl)pyrimidin-2-yl, 5,7-dihydrofuro[3,4-d]pyrimidin-2-yl, 5-chloro-3-fluoropyridin-2-yl, 5-chloropyridin-2-yl, 5-chloropyrimidin-2-yl, 5-cyano-3-fluoropyridin-2-yl, 5-cyanobenzo[d]oxazol-2-yl, 5-cyanopyridin-2-yl, 5-cyanopyrimidin-2-yl, 5-cyclopropylpyrimidin-2-yl, 5-ethylpyrimidin-2-yl, 5-fluoro-4-methylpyrimidin-2-yl, 5-fluoropyridin-2-yl, 5-fluoropyrimidin-2-yl, 5-fluoropyrimidin-4-yl, 5-methoxypyrimidin-2-yl, 5-methyl-2-oxo-1,2-dihydropyridin-3-yl, 5-methylpyrimidin-2-yl, 5-pyrazol-1-ylpyrimidin-2-yl, 6,7-dihydro-5H-pyrrolo[1,2-b][1,2,4]triazol-2-yl, 5-fluorothiazol-2-yl, 6-chloropyridazin-3-yl, 6-cyanobenzo[d]oxazol-2-yl, 6-methylpyrazin-2-yl, 6-methylpyridin-2-yl, 6-oxo-1,6-dihydropyrimidin-2-yl, benzo[d]oxazol-2-yl, benzo[d]oxazol-5-yl, benzo[d]thiazol-5-yl, benzo[d]thiazol-6-yl, cyclobutylmethyl, imidazo[1,2-a]pyrazin-6-yl, imidazo[1,2-a]pyridin-5-yl, imidazo[1,2-a]pyridin-8-yl, imidazo[1,2-b]pyridazin-6-yl, imidazo[1,5-a]pyridin-6-yl, isoquinolin-4-yl, isoquinolin-6-yl, isoquinolin-7-yl, isoquinolin-8-yl, isoxazolo[4,5-b]pyridin-5-yl, isoxazolo[5,4-b]pyridin-6-yl, oxazol-2-ylmethyl, oxazolo[4,5-b]pyridin-2-yl, oxazolo[4,5-c]pyridin-2-yl, oxazolo[5,4-b]pyridin-2-yl, oxazolo[5,4-c]pyridin-2-yl, oxetan-3-ylmethyl, phenyl, pyrazolo[1,5-a]pyrimidin-5-yl, pyridin-4-ylmethyl, pyrimidin-2-yl, quinazolin-2-yl, quinolin-2-yl, quinolin-3-yl, quinolin-5-yl, quinolin-6-yl, or spiro[2.3]hexan-5-yl.

In certain embodiments. R⁴ and R⁵ together form a heterocyclyl or heteroaryl ring optionally substituted with one to eight Z¹. In certain embodiments, R⁴ and R⁵ together form a heterocyclyl ring optionally substituted with one to eight Z¹. In certain embodiments, R⁴ and R⁵ together form a 3-(1-hydroxy-1-methyl-ethyl)pyrrolidin-1-yl, 3-hydroxy-3-methyl-pyrrolidin-1-yl, 5,7-dihydro-6H-pyrrolo[3,4-b]pyridin-6-yl, or 2,3-dihydro-1H-pyrrolo[2,3-b]pyridin-1-yl. In certain embodiments, R⁴ and R⁵ together form a 3-(1-hydroxy-1-methyl-ethyl)pyrrolidin-1-yl or 3-hydroxy-3-methyl-pyrrolidin-1-yl.

In certain embodiments. R⁵ is not C₁₋₆ alkyl, or when R⁴ and R⁵ together form a heterocyclyl or heteroaryl ring, the ring is not pyrrolidine, piperidine, morpholine, piperazine, N-lower alkylpiperazine or N-6-hydroxyethylpiperazine. In certain embodiments, R⁵ is not C₁₋₆ alkyl, certain embodiments, R⁴ and R⁵ together form a heterocyclyl or heteroaryl ring optionally substituted with one to eight Z¹, provided the heterocyclyl or heteroaryl ring is not unsubstituted pyrrolidine, unsubstituted piperidine, unsubstituted morpholine, unsubstituted piperazine, N-lower alkylpiperazine or N-6-hydroxyethylpiperazine.

In certain embodiments, R⁹ is hydrogen or C₁₋₆ alkyl. In certain embodiments, R⁹ is hydrogen or methyl and R¹⁰ is hydrogen. In certain embodiments, R⁹ is hydrogen. In certain embodiments, R¹⁰ is hydrogen. In certain embodiments, R⁹ and R¹⁰ are hydrogen. In certain embodiments, R⁹ is methyl and R¹⁰ is hydrogen.

In certain embodiments, provided is a compound represented by Formula II:

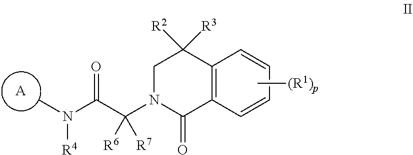

wherein:
R¹, R², R³, R⁴, R⁶, and R⁷ are each independently as defined herein;
p is 1, 2, 3, or 4; and
ring A is C₃₋₁₀ cycloalkyl, heterocyclyl, aryl, or heteroaryl; wherein the C₃₋₁₀ cycloalkyl, heterocyclyl, aryl, or heteroaryl is optionally substituted with one to eight Z¹.

In certain embodiments, provided is a compound represented by Formula III:

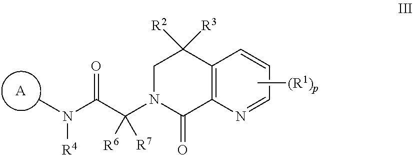

wherein:
R¹, R², R³, R⁴, R⁶, and R⁷ are each independently as defined herein;
p is 1, 2, or 3; and
ring A is C₃₋₁₀ cycloalkyl, heterocyclyl, aryl, or heteroaryl; wherein the C₃₋₁₀ cycloalkyl, heterocyclyl, aryl, or heteroaryl is optionally substituted with one to eight Z¹.

In certain embodiments, provided is a compound represented by Formula IV:

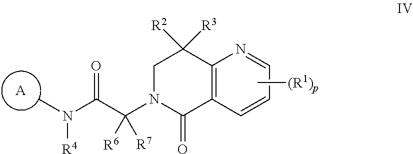

wherein:
R¹, R², R³, R⁴, R⁶, and R⁷ are each independently as defined herein;
p is 1, 2, or 3; and
ring A is C₃₋₁₀ cycloalkyl, heterocyclyl, aryl, or heteroaryl; wherein the C₃₋₁₀ cycloalkyl, heterocyclyl, aryl, or heteroaryl is optionally substituted with one to eight Z¹.

In certain embodiments, provided is a compound represented by Formula V:

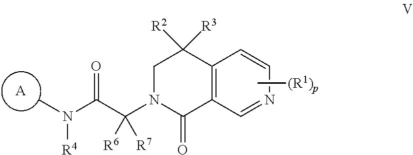

wherein:
R¹, R², R³, R⁴, R⁶, and R⁷ are each independently as defined herein;
p is 1, 2, or 3; and
ring A is C₃₋₁₀ cycloalkyl, heterocyclyl, aryl, or heteroaryl; wherein the C₃₋₁₀ cycloalkyl, heterocyclyl, aryl, or heteroaryl is optionally substituted with one to eight Z¹.

In certain embodiments, provided is a compound represented by Formula VI:

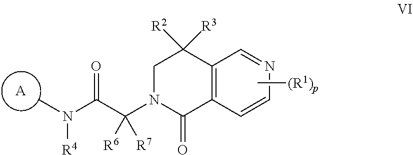

wherein:
R¹, R², R³, R⁴, R⁶, and R⁷ are each independently as defined herein;
p is 1, 2, or 3; and
ring A is C₃₋₁₀ cycloalkyl, heterocyclyl, aryl, or heteroaryl; wherein the C₃₋₁₀ cycloalkyl, heterocyclyl, aryl, or heteroaryl is optionally substituted with one to eight Z¹.

In certain embodiments. R⁴ is hydrogen or methyl. In certain embodiments, R⁴ is hydrogen.

In certain embodiments, R⁶ is hydrogen, halo, cyano, C₁₋₆ alkyl, C₂₋₆ alkenyl, C₂₋₆ alkynyl, C₁₋₆ haloalkyl, C₂₋₆ heteroalkyl, C₃₋₁₀ cycloalkyl, or heterocyclyl. In certain embodiments, R⁶ is hydrogen.

In certain embodiments. R⁷ is hydrogen, halo, cyano, hydroxy, C₁₋₆ alkyl, C₂₋₆ alkenyl, C₂₋₆ alkynyl, C₁₋₆ haloalkyl, C₂₋₆ heteroalkyl, C₃₋₁₀ cycloalkyl, or heterocyclyl. In certain embodiments, R⁷ is hydrogen.

In certain embodiments. R⁶ and R⁷ are hydrogen.

In certain embodiments. R⁶ and R⁷ join to form a C₃₋₁₀ cycloalkyl.

In certain embodiments, R⁴ is hydrogen; R⁶ is hydrogen; and R⁷ is hydrogen.

In certain embodiments, provided is a compound represented by Formula VII:

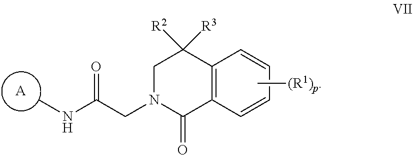

wherein:
R¹, R², and R³ are each independently as defined herein;
p is 1, 2, 3, or 4; and
ring A is C₃₋₁₀ cycloalkyl, heterocyclyl, aryl, or heteroaryl; wherein the C₃₋₁₀ cycloalkyl, heterocyclyl, aryl, or heteroaryl is optionally substituted with one to eight Z¹.

In certain embodiments, p is 1. In certain embodiments, p is 2. In certain embodiments, p is 1 or 2. In certain embodiments, p is 3. In certain embodiments, p is 4.

In certain embodiments, each R¹ is independently halo, cyano, C₁₋₆ alkyl, C₂₋₆ alkenyl, C₂₋₆ alkynyl, C₁₋₆ alkoxy, C₁₋₆haloalkyl, C₁₋₆ haloalkoxy, C₃₋₁₀ cycloalkyl, or heterocyclyl, wherein the C₁₋₆ alkyl, C₂₋₆ alkenyl, and C₃₋₁₀ cycloalkyl are independently optionally substituted with one to eight Z¹; or any two adjacent R¹ together with the atoms to which they are attached form a cycloalkyl, heterocyclyl, aryl, or heteroaryl ring.

In certain embodiments, each R¹ is independently fluoro, bromo, chloro, iodo, cyano, ethyl, vinyl, difluoromethyl, trifluoromethyl, 1-fluoroethyl, 1,1-difluoroethyl, methoxy, fluoromethoxy, difluoromethoxy, cyclopropyl, cyclobutyl, cyclopropylmethyl, oxetan-3-yl, 2,2-difluorocycloprop-1-yl, 1-cyanocyclopropyl, 1-methylcyclopropyl, 1-fluoro-2-(trifluoromethyl)cyclopropyl, ethynyl, 1-fluorovinyl, 1-fluorocyclopropyl, 2-fluorocyclopropyl, or 1,2-difluorocyclopropyl; or two adjacent R¹ together with the atoms to which they are attached form a thiophene.

In certain embodiments, each R¹ is independently halo, cyano, C₁₋₆ alkyl, C₂₋₆ alkenyl, C₂₋₆ alkynyl, C₁₋₆ alkoxy, C₁₋₆ haloalkyl, C₁₋₆ haloalkoxy, or C₃₋₁₀ cycloalkyl, wherein the C₂₋₆ alkenyl or C₃₋₁₀ cycloalkyl is independently optionally substituted with one to eight Z¹. In certain embodiments, each R¹ is independently halo, cyano, C₁₋₆ alkyl, C₁₋₆ alkoxy, C₁₋₆haloalkyl, C₁₋₆ haloalkoxy, or C₃₋₁₀ cycloalkyl, wherein the C₃₋₁₀ cycloalkyl is independently optionally substituted with one to eight Z¹. In certain embodiments, each R¹ is independently halo, cyano, C₁₋₆ alkyl, C₂₋₆ alkenyl, C₂₋₆ alkynyl, C₁₋₆ alkoxy, C₁₋₆ haloalkyl, C₁₋₆ haloalkoxy, or C₃₋₁₀ cycloalkyl, wherein the C₂₋₆ alkenyl or C₃₋₁₀ cycloalkyl is independently optionally substituted with one to eight Z¹, wherein each is independently selected from halo, cyano and C₁₋₆ alkyl.

In certain embodiments, each R¹ is independently halo, cyano, C₁₋₆ alkyl, C₁₋₆ alkoxy, C₁₋₆ haloalkoxy, C₁₋₆haloalkyl, or C₃₋₁₀ cycloalkyl. In certain embodiments, each R¹ is independently halo, cyano, C₁₋₆ haloalkoxy, C₁₋₆ haloalkyl, or C₃₋₁₀ cycloalkyl. In certain embodiments, each R¹ is independently halo, cyano, C₁₋₆ alkyl, C₁₋₆ alkoxy, or C₁₋₆ haloalkyl. In certain embodiments, each R¹ is independently halo or C₁₋₆ alkyl.

In certain embodiments, each R¹ is independently fluoro, bromo, chloro, iodo, cyano, difluoromethyl, trifluoromethyl, 1,1-difluoroethyl, methoxy, fluoromethoxy, difluoromethoxy, cyclopropyl, cyclobutyl, 2,2-difluorocycloprop-1-yl, 1-cyanocyclopropyl, and 1-methylcyclopropyl, ethynyl, 1-fluorovinyl, 1-fluorocyclopropyl, or 1,2-difluorocyclopropyl. In certain embodiments, each R¹ is independently fluoro, bromo, chloro, iodo, cyano, difluoromethyl, trifluoromethyl, 1,1-difluoroethyl, methoxy, fluoromethoxy, difluoromethoxy, cyclopropyl, cyclobutyl, 2,2-difluorocycloprop-1-yl, 1-cyanocyclopropyl, and 1-methylcyclopropyl. In certain embodiments, each R¹ is independently fluoro, bromo, —CH₃, —OCHF₂, —CF₃, or cyclopropyl. In certain embodiments, each R¹ is independently fluoro, bromo, or —CH₃. In certain embodiments, each R¹ is independently halo. In certain embodiments, each R¹ is independently bromo. In certain embodiments, each R¹ is independently halo or —CF₃.

In certain embodiments, p is 1; and each R¹ is independently halo, cyano, C₁₋₆ alkyl, C₁₋₆ alkoxy, or C₁₋₆ haloalkyl. In certain embodiments, p is 2; and each R¹ is independently halo, cyano, C₁₋₆ alkyl, C₁₋₆ alkoxy, or C₁₋₆ haloalkyl. In certain embodiments, p is 1 or 2; and each R¹ is independently halo, cyano, C₁₋₆ alkyl, C₁₋₆ alkoxy, or C₁₋₆ haloalkyl.

In certain embodiments. R² and R³ together form a C₃₋₁₀ cycloalkyl or heterocyclyl ring; wherein the C₃₋₁₀ cycloalkyl or heterocyclyl is independently optionally substituted with one to eight Z¹. In certain embodiments, R² and R³ together form a C₃₋₁₀ cycloalkyl ring optionally substituted with one to eight Z¹. In certain embodiments, R² and R³ together form a C₃₋₁₀ cycloalkyl optionally substituted with halo, cyano, C₁₋₆ alkyl or C₁₋₆ haloalkyl. In certain embodiments, R² and R³ together form a C₃₋₁₀ cycloalkyl optionally substituted with halo, C₁₋₆ alkyl or C₁₋₆ haloalkyl. In certain embodiments, R² and R³ together form a C₃₋₁₀ cycloalkyl optionally substituted with fluoro, methyl or trifluoromethyl. In certain embodiments, R² and R³ together form a C₃₋₁₀ cycloalkyl ring optionally substituted with C₁₋₆ alkyl. In certain embodiments, R² and R³ together form a C₃₋₁₀ cycloalkyl ring optionally substituted with methyl. In certain embodiments, R² and R³ together form a heterocyclyl ring optionally substituted with one to eight Z¹. In certain embodiments, R² and R³ together form a C₃₋₁₀ cycloalkyl or heterocyclyl ring. In certain embodiments, R² and R³ together form an unsubstituted C₃₋₁₀ cycloalkyl ring. In certain embodiments, R² and R³ together form an unsubstituted cyclopropyl ring. In certain embodiments, R² and R³ together form an unsubstituted heterocyclyl ring.

In certain embodiments, R² is C₁₋₆ alkyl, C₁₋₆ haloalkyl, or —OR¹¹, wherein R¹¹ is C₁₋₆ alkyl optionally substituted with one to five Z^{1a}. In certain embodiments, R² is C₁₋₆ alkyl or C₁₋₆ haloalkyl, and R³ is hydrogen or C₁₋₆ alkyl. In certain embodiments, R² is C₁₋₆ alkyl or C₁₋₆ haloalkyl. In certain embodiments, R² is C₁₋₆ alkyl. In certain embodiments, R² is methyl or ethyl.

In certain embodiments, R² is —C(R¹⁴)₂R¹; each R¹⁴ and R¹⁵ are independently hydrogen, halo, C₁₋₄ alkyl, or C₁₋₄ haloalkyl. In certain embodiments, R² is —C(R¹⁴)₂R⁵; each R¹⁴ is independently hydrogen, halo, C₁₋₄ alkyl, or C₁₋₄haloalkyl, and R¹⁵ is hydrogen.

In certain embodiments, R³ is hydrogen, C₁₋₆alkyl or C₁₋₆ haloalkyl. In certain embodiments, R³ is hydrogen or C₁₋₆ alkyl. In certain embodiments, R³ is C₁₋₆ alkyl. In certain embodiments, R³ is hydrogen or methyl. In certain embodiments, R³ is hydrogen. In certain embodiments, R³ is methyl.

In certain embodiments, R² is C₁₋₆ alkyl; R³ is hydrogen, or C₁₋₆ alkyl; or R² and R³ together form a C₃₋₁₀ cycloalkyl ring optionally substituted with C₁₋₆ alkyl. In certain embodiments, R² and R³ are C₁₋₆ alkyl.

In certain embodiments, provided is a compound represented by Formula VIII:

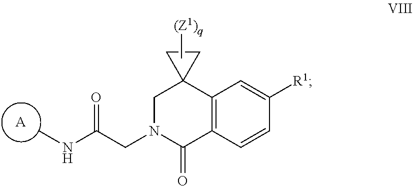

wherein q is 0, 1, 2, 3, or 4, and R¹ and ring A are each independently as defined herein.

In certain embodiments, provided is a compound represented by Formula IX:

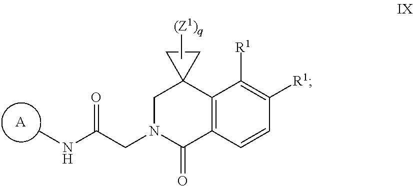

wherein q is 0, 1, 2, 3, or 4, and R¹ and ring A are each independently as defined herein.

In certain embodiments, q is 1. In certain embodiments, q is 2. In certain embodiments, q is 3.

In certain embodiments, q is 4. In certain embodiments, each Z¹ is independently halo, cyano, C₁₋₆ alkyl, or C₁₋₆ haloalkyl. In certain embodiments, q is 1 or 2; and each Z¹ is independently halo, cyano, C₁₋₆ alkyl, or C₁₋₆ haloalkyl.

In certain embodiments, provided is a compound represented by Formula VIIIA:

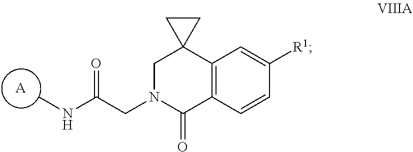

wherein R¹ and ring A are each independently as defined herein.

In certain embodiments. R¹ is halo, cyano, C₁₋₆ alkyl, C₁₋₆ alkoxy, or C₁₋₆ haloalkyl. In certain embodiments, R¹ is halo. In certain embodiments, R¹ is bromo. In certain embodiments, R¹ is —CF₃.

In certain embodiments, provided is a compound represented by Formula IXA:

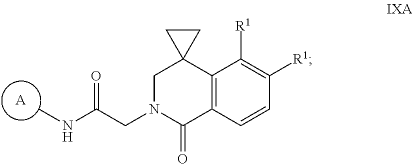

wherein R¹ and ring A are each independently as defined herein.

In certain embodiments, each R¹ is independently halo, cyano, C₁₋₆ alkyl, C₁₋₆ alkoxy, or C₁₋₆ haloalkyl. In certain embodiments, each R¹ is independently fluoro, bromo, —CH₃, or —CF₃. In certain embodiments, each R¹ is independently halo. In certain embodiments, each R¹ is independently bromo. In certain embodiments, each R¹ is independently halo or —CF₃.

In certain embodiments, R⁵ or ring A is C₃₋₁₀ cycloalkyl, heterocyclyl, aryl, or heteroaryl; wherein the C₃₋₁₀ cycloalkyl, heterocyclyl, aryl, or heteroaryl is independently optionally substituted with one to five Z¹. In certain embodiments, R⁵ or ring A is C₃₋₁₀ cycloalkyl, heterocyclyl or heteroaryl; wherein the C₃₋₁₀ cycloalkyl, heterocyclyl or heteroaryl is independently optionally substituted with one to five Z¹.

In certain embodiments, R⁵ or ring A is pyrazolo[3,4-d]pyrimidinyl, imidazo[1,2-b]pyridazinyl, pyrazolo[1,5-a]pyridinyl, 5H-1,3-oxazolyl. 4,5,6,7-tetrahydro-1,3-benzoxazolyl, 1,3-benzoxazolyl, 3-oxopyrazinyl, 4-azaspiro[2.5]octanyl, 5-oxopyrazinyl, 4H-1,3-oxazolyl, 5,6,7,8-tetrahydro-[1,2,4]triazolo[1,5-a]pyridinyl, 5,6-dihydrofuro[2,3-d]pyrimidinyl, 6,7-dihydro-4H-pyrazolo[5,1-c][1,4]oxazinyl, 6,8-dihydro-5H-pyrano[3,4-d]pyrimidinyl, 7,8-dihydro-5H-pyrano[4,3-d]pyrimidinyl, imidazo[1,2-c]pyrimidinyl, oxopyridazinyl, 2-oxopyrrolidinyl, azaspiro[3.3]heptanyl, azabicyclo[2.2.2]octanyl, [1,2,4]triazolo[4,3-a]pyrazinyl, [1,3]thiazolo[5,4-d]pyrimidinyl, pyrrolidinyl, 1,2,4-benzotriazinyl, 1,2-benzothiazolyl, 1,2-benzoxazolyl, pyrazinyl, [1,2,4]triazolo[1,5-a]pyrazinyl, [1,2,4]triazolo[1,5-a]pyridinyl, [1,2,4]triazolo[4,3-a]pyridinyl, 1,2,4-triazolyl, 1,3,5-triazinyl, 1,6-naphthyridinyl, 1,7-naphthyridinyl, 1,8-naphthyridinyl, 1H-benzo[d][1,2,3]triazolyl, 1H-benzo[d]imidazolyl, 1H-indazolyl, 1H-pyrrolo[2,3-b]pyridinyl, 2,3-dihydro-1H-indenyl, 2,3-dihydro-1H-pyrrolo[2,3-b]pyridinyl, 2,3-dihydrobenzofuranyl, 2-oxo-1,2-dihydropyridinyl, 2-oxopiperidyl, 2-oxo-pyrrolidinyl, 4,5,6,7-tetrahydroindazolyl, 4,5,6,7-tetrahydropyrazolo[1,5-a]pyridinyl, 5,7-dihydro-6H-pyrrolo[3,4-b]pyridinyl, 5,7-dihydrofuro[3,4-d]pyrimidinyl, 6,7-dihydro-5H-pyrrolo[1,2-b][1,2,4]triazolyl, thiazolyl, 6-oxo-1,6-dihydropyrimidinyl, benzo[d][1.3]dioxolyl, benzo[d]oxazolyl, benzo[d]thiazolyl, bicyclo[1.1.1]pentanyl, bicyclo[2.2.2]octanyl, cyclobutyl, cyclohexyl, cyclopropyl, imidazo[1,2-a]pyrazinyl, imidazo[1,2-a]pyridinyl, imidazo[1,2-b]pyridazinyl, imidazo[1,5-a]pyridinyl, indazolyl, indolyl, isoquinolinyl, isoxazolo[4,5-b]pyridinyl, isoxazolo[5,4-b]pyridinyl, isoxazolyl, imidazo[1,2-a]pyridinyl, oxabicyclo[2.1.1]hexanyl, oxabicyclo[2.2.1]heptanyl, oxabicyclo[2.2.2]octanyl, oxaspiro[3.3]heptanyl, oxazolo[4,5-b]pyridinyl, oxazolo[4,5-c]pyridinyl, oxazolo[5,4-b]pyridinyl, oxazolo[5,4-c]pyridinyl, phenyl, piperidinyl, pyrazolo[1,5-a]pyrimidinyl, pyrazolo[4,3-b]pyridinyl, pyrazolyl, pyridazinyl, pyridinyl, pyrimidinyl, quinazolinyl, quinolinyl, spiro[2.3]hexanyl, or tetrahydropyranyl; wherein each is independently optionally substituted with one to five Z¹.

In certain embodiments, R⁵ or ring A is [1,2,4]triazolo[1,5-a]pyrazinyl, [1,2,4]triazolo[1,5-a]pyridinyl, [1,2,4]triazolo[4,3-a]pyridinyl, 1,2,4-triazolyl, 1,3,5-triazinyl, 1,6-naphthyridinyl, 1,7-naphthyridinyl, 1,8-naphthyridinyl, 1H-benzo[d][1,2,3]triazolyl, 1H-benzo[d]imidazolyl, 1H-indazolyl, 1H-pyrrolo[2,3-b]pyridinyl, 2,3-dihydro-1H-indenyl, 2,3-dihydro-1H-pyrrolo[2,3-b]pyridinyl, 2,3-dihydrobenzofuranyl, 2-oxo-1,2-dihydropyridinyl, 2-oxopiperidyl, 2-oxo-pyrrolidinyl, 4,5,6,7-tetrahydroindazolyl, 4,5,6,7-tetrahydropyrazolo[1,5-a]pyridinyl, 5,7-dihydro-6H-pyrrolo[3,4-b]pyridinyl, 5,7-dihydrofuro[3,4-d]pyrimidinyl, 6,7-dihydro-5H-pyrrolo[1,2-b][1,2,4]triazolyl, thiazolyl, 6-oxo-1,6-dihydropyrimidinyl, benzo[d][1,3]dioxolyl, benzo[d]oxazolyl, benzo[d]thiazolyl, bicyclo[1.1.1]pentanyl, bicyclo[2.2.2]octanyl, cyclobutyl, cyclohexyl, cyclopropyl, imidazo[1,2-a]pyrazinyl, imidazo[1,2-a]pyridinyl, imidazo[1,2-b]pyridazinyl, imidazo[1,5-a]pyridinyl, indazolyl, indolyl, isoquinolinyl, isoxazolo[4,5-b]pyridinyl, isoxazolo[5,4-b]pyridinyl, isoxazolyl, imidazo[1,2-a]pyridinyl, oxabicyclo[2.1.1]hexanyl, oxabicyclo[2.2.1]heptanyl, oxabicyclo[2.2.2]octanyl, oxaspiro[3.3]heptanyl, oxazolo[4,5-b]pyridinyl, oxazolo[4,5-c]pyridinyl, oxazolo[5,4-b]pyridinyl, oxazolo[5,4-c]pyridinyl, phenyl, piperidinyl, pyrazolo[1,5-a]pyrimidinyl, pyrazolo[4,3-b]pyridinyl, pyrazolyl, pyridazinyl, pyridinyl, pyrimidinyl, quinazolinyl, quinolinyl, spiro[2.3]hexanyl, or tetrahydropyranyl; wherein each is independently optionally substituted with one to five Z¹.

In certain embodiments. R⁵ or ring A is [1,2,4]triazolo[1,5-a]pyridinyl, [1,2,4]triazolo[4,3-a]pyridinyl, 1,2,4-triazolyl, 1,3,5-triazinyl, 1,6-naphthyridinyl, 1,7-naphthyridinyl, 1,8-naphthyridinyl, 1H-benzo[d][1,2,3]triazolyl, 1H-benzo[d]imidazolyl, 1H-indazolyl, 1H-pyrrolo[2,3-b]pyridinyl, 2,3-dihydro-1H-indenyl, 2,3-dihydro-1H-pyrrolo[2,3-b]pyridinyl, 2,3-dihydrobenzofuranyl, 2-oxo-1,2-dihydropyridinyl, 2-oxopiperidyl, 2-oxo-pyrrolidinyl, 4,5,6,7-tetrahydroindazolyl, 4,5,6,7-tetrahydropyrazolo[1,5-a]pyridinyl, 5,7-dihydro-6H-pyrrolo[3,4-b]pyridinyl, 5,7-dihydrofuro[3,4-d]pyrimidinyl, 6,7-dihydro-5H-pyrrolo[1,2-b][1,2,4]triazolyl, thiazolyl, 6-oxo-1,6-dihydropyrimidinyl, benzo[d][1,3]dioxolyl, benzo[d]oxazolyl, benzo[d]thiazolyl, bicyclo[1.1.1]pentanyl, bicyclo[2.2.2]octanyl, cyclobutyl, cyclohexyl, cyclopropyl, imidazo[1,2-a]pyrazinyl, imidazo[1,2-a]pyridinyl, imidazo[1,2-b]pyridazinyl, imidazo[1,5-a]pyridinyl, indazolyl, indolyl, isoquinolinyl, isoxazolo[4,5-b]pyridinyl, isoxazolo[5,4-b]pyridinyl, isoxazolyl, imidazo[1,2-a]pyridinyl, oxabicyclo[2.1.1]hexanyl, oxabicyclo[2.2.1]heptanyl, oxabicyclo[2.2.2]octanyl, oxaspiro[3.3]heptanyl, oxazolo[4,5-b]pyridinyl, oxazolo[4,5-c]pyridinyl, oxazolo[5,4-b]pyridinyl, oxazolo[5,4-c]pyridinyl, phenyl, piperidinyl, pyrazolo[1,5-a]pyrimidinyl, pyrazolo[4,3-b]pyridinyl, pyrazolyl, pyridazinyl, pyridinyl, pyrimidinyl, quinazolinyl, quinolinyl, spiro[2.3]hexanyl, or tetrahydropyranyl; wherein each is independently optionally substituted with one to five Z¹.

In certain embodiments, R⁵ or ring A is pyrimidinyl, pyridinyl, pyridazinyl, bicyclo[1.1.1]pentanyl, piperidinyl, oxabicyclo[2.2.1]heptanyl, cyclohexyl, cyclobutyl, 4,5,6,7-tetrahydropyrazolo[1,5-a]pyridinyl, tetrahydropyranyl, 2-oxopiperidyl, spiro[2.3]hexanyl, indazolyl, indolyl, 4,5,6,7-tetrahydroindazolyl, 5-oxo-pyrrolidin-3-yl, oxabicyclo[2.2.2]octanyl, oxaspiro[3.3]heptanyl, oxabicyclo[2.1.1]hexanyl, or bicyclo[2.2.2]octanyl; wherein each is independently optionally substituted with one to five Z¹.

In certain embodiments, R⁵ or ring A is pyrimidinyl, pyridinyl, pyridazinyl, bicyclo[1.1.1]pentanyl, piperidinyl, oxabicyclo[2.2.1]heptanyl, cyclohexyl, cyclobutyl, 4,5,6,7-tetrahydropyrazolo[1,5-a]pyridinyl, tetrahydropyranyl, 2-oxopiperidyl, or spiro[2.3]hexanyl; wherein each is independently optionally substituted with one to five Z¹.

In certain embodiments. R⁵ is 5-fluoropyrimidin-4-yl, 5-fluoropyrimidin-2-yl, 5-cyanopyrimidin-2-yl, 5-chloropyrimidin-2-yl, pyrimidin-2-yl, 5-fluoropyrimidin-4-yl, 5-cyano-3-fluoropyridin-2-yl, 5-chloro-3-fluoropyridin-2-yl, 3-fluoro-5-(trifluoromethyl)pyridin-2-yl, 6-chloropyridazin-3-yl, 3-fluoropyridin-4-yl, 3,5-difluoro-2-pyridyl, 3-(trifluoromethyl)-1-bicyclo[1.1.1]pentanyl, 1-cyclobutylpiperidin-3-yl, 3-cyano-1-bicyclo[1.1.1]pentanyl, 4,4-difluorocyclohexyl, (1R,2R,4S)-7-oxabicyclo[2.2.1]heptan-2-yl, (1S,2R,4R)-7-oxabicyclo[2.2.1]heptan-2-yl, 1-ethylpiperidin-3-yl, 3-hydroxy-3-methylcyclobutyl, 4,5,6,7-tetrahydropyrazolo[1,5-a]pyridin-5-yl, 1-methyl-6-oxo-3-piperidyl, 1-methyl-2-oxo-4-piperidyl, spiro[2.3]hexan-5-yl, 2-cyclopropyltetrahydropyran-4-yl, 1-(2-methoxyethyl)-3-piperidyl, 1-cyclobutylpiperidin-3-yl, 5-(difluoromethoxy)-2-pyridyl, 3-fluoro-5-formylpyridin-2-yl, 1-ethyl-6-oxo-3-piperidyl, 5-pyrazol-1-ylpyrimidin-2-yl, 5-cyanopyridin-2-yl, 1-methyl-6-oxo-3-pyridyl, 1H-indazol-6-yl, 1H-indol-6-yl, 4,5,6,7-tetrahydro-1H-indazol-6-yl, 1-(methoxycarbonyl)piperidin-3-yl, 1-methyl-5-oxo-pyrrolidin-3-yl, 2-oxabicyclo[2.2.2]octan-4-yl, 2-oxaspiro[3.3]heptan-6-yl. 1-methyl-2-oxabicyclo[2.1.1]hexan-4-yl, 3-(1-hydroxy-1-methyl-ethyl)-1-bicyclo[1.1.1]pentanyl, 3-methylcyclobutyl, 3-(trifluoromethyl)cyclobutyl, 3-hydroxy-3-(trifluoromethyl)cyclobutyl, 1-bicyclo[2.2.2]octanyl, 4-hydroxy-1-bicyclo[2.2.2]octanyl, 3-hydroxycyclohexyl, 3-(difluoromethoxy)cyclobutyl, 3-(hydroxymethyl)cyclobutyl, 3-cyanocyclobutyl, 3,3-difluorocyclobutyl, 3-hydroxy-3-methylbutyl, or 2-hydroxy-2-methyl-propyl.

In certain embodiments, R⁵ or ring A is 5-fluoropyrimidin-4-yl, 5-fluoropyrimidin-2-yl, 5-cyanopyrimidin-2-yl, 5-chloropyrimidin-2-yl, pyrimidin-2-yl, 5-fluoropyrimidin-4-yl, 5-cyano-3-fluoropyridin-2-yl, 5-chloro-3-fluoropyridin-2-yl, 3-fluoro-5-(trifluoromethyl)pyridin-2-yl, 6-chloropyridazin-3-yl, 3-fluoropyridin-4-yl, 3,5-difluoro-2-pyridyl, 3-(trifluoromethyl)-1-bicyclo[1.1.1]pentanyl, 1-cyclobutylpiperidin-3-yl, 3-cyano-1-bicyclo[1.1.1]pentanyl, 4,4-difluorocyclohexyl, (1R,2R,4S)-7-oxabicyclo[2.2.1]heptan-2-yl, (1S,2R,4R)-7-oxabicyclo[2.2.1]heptan-2-yl, 1-ethylpiperidin-3-yl, 3-hydroxy-3-methylcyclobutyl, 4,5,6,7-tetrahydropyrazolo[1,5-a]pyridin-5-yl, 1-methyl-6-oxo-3-piperidyl, 1-methyl-2-oxo-4-piperidyl, spiro[2.3]hexan-5-yl, 2-cyclopropyltetrahydropyran-4-yl, 1-(2-methoxyethyl)-3-piperidyl, 1-cyclobutylpiperidin-3-yl, 5-(difluoromethoxy)-2-pyridyl, 3-fluoro-5-formylpyridin-2-yl, 1-ethyl-6-oxo-3-piperidyl, 5-pyrazol-1-ylpyrimidin-2-yl, 5-cyanopyridin-2-yl, 1-methyl-6-oxo-3-pyridyl, 1H-indazol-6-yl, 1H-indol-6-yl, 4,5,6,7-tetrahydro-1H-indazol-6-yl, 1-(methoxycarbonyl)piperidin-3-yl, 1-methyl-5-oxo-pyrrolidin-3-yl, 2-oxabicyclo[2.2.2]octan-4-yl, 2-oxaspiro[3.3]heptan-6-yl. 1-methyl-2-oxabicyclo[2.1.1]hexan-4-yl, 3-(1-hydroxy-1-methyl-ethyl)-1-bicyclo[1.1.1]pentanyl, 3-methylcyclobutyl, 3-(trifluoromethyl)cyclobutyl, 3-hydroxy-3-(trifluoromethyl)cyclobutyl, 1-bicyclo[2.2.2]octanyl, 4-hydroxy-1-bicyclo[2.2.2]octanyl, 3-hydroxycyclohexyl, 3-(difluoromethoxy)cyclobutyl, 3-(hydroxymethyl)cyclobutyl, 3-cyanocyclobutyl, or 3,3-difluorocyclobutyl. In certain embodiments, R⁵ or ring A is 5-fluoropyrimidin-4-yl, 5-fluoropyrimidin-2-yl, 5-cyanopyrimidin-2-yl, 5-chloropyrimidin-2-yl, pyrimidin-2-yl, 5-fluoropyrimidin-4-yl, 5-cyano-3-fluoropyridin-2-yl, 5-chloro-3-fluoropyridin-2-yl, 3-fluoro-5-(trifluoromethyl)pyridin-2-yl, 6-chloropyridazin-3-yl, 3-fluoropyridin-4-yl, 3,5-difluoro-2-pyridyl, 3-(trifluoromethyl)-1-bicyclo[1.1.1]pentanyl, 1-cyclobutylpiperidin-3-yl, 3-cyano-1-bicyclo[1.1.1]pentanyl, 4,4-difluorocyclohexyl, (1R,2R,4S)-7-oxabicyclo[2.2.1]heptan-2-yl, (1S,2R,4R)-7-oxabicyclo[2.2.1]heptan-2-yl, 1-ethylpiperidin-3-yl, 3-hydroxy-3-methylcyclobutyl, 4,5,6,7-tetrahydropyrazolo[1,5-a]pyridin-5-yl, 1-methyl-6-oxo-3-piperidyl, 1-methyl-2-oxo-4-piperidyl, spiro[2.3]hexan-5-yl, 2-cyclopropyltetrahydropyran-4-yl, 1-(2-methoxyethyl)-3-piperidyl, or 1-cyclobutylpiperidin-3-yl.

In certain embodiments, R⁵ or ring A is C₃₋₁₀ cycloalkyl optionally substituted with one to five Z¹.

In certain embodiments. R⁵ or ring A is bicyclo[1.1.1]pentanyl, cyclohexyl, cyclobutyl, spiro[2.3]hexanyl, or bicyclo[2.2.2]octanyl; wherein each is independently optionally substituted with one to five Z¹. In certain embodiments, R⁵ or ring A is bicyclo[1.1.1]pentanyl, cyclohexyl, cyclobutyl, or spiro[2.3]hexanyl; wherein each is independently optionally substituted with one to five Z¹.

In certain embodiments. R⁵ or ring A is 3-(trifluoromethyl)-1-bicyclo[1.1.1]pentanyl, 3-cyano-1-bicyclo[1.1.1]pentanyl, 4,4-difluorocyclohexyl, 3-hydroxy-3-methylcyclobutyl, spiro[2.3]hexan-5-yl, 3-(1-hydroxy-1-methyl-ethyl)-1-bicyclo[1.1.1]pentanyl, 3-methylcyclobutyl, 3-(trifluoromethyl)cyclobutyl, 3-hydroxy-3-(trifluoromethyl)cyclobutyl, 1-bicyclo[2.2.2]octanyl, 4-hydroxy-1-bicyclo[2.2.2]octanyl, 3-hydroxycyclohexyl, 3-(difluoromethoxy)cyclobutyl, 3-(hydroxymethyl)cyclobutyl, 3-cyanocyclobutyl, or 3,3-difluorocyclobutyl. In certain embodiments, R⁵ or ring A is 3-(trifluoromethyl)-1-bicyclo[1.1.1]pentanyl, 3-cyano-1-bicyclo[1.1.1]pentanyl, 4,4-difluorocyclohexyl, 3-hydroxy-3-methylcyclobutyl, or spiro[2.3]hexan-5-yl; wherein each is independently optionally substituted with one to five Z¹. In certain embodiments, R⁵ or ring A is 3-(trifluoromethyl)-1-bicyclo[1.1.1]pentanyl, 3-cyano-1-bicyclo[1.1.1]pentanyl, 4,4-difluorocyclohexyl, 3-hydroxy-3-methylcyclobutyl, or spiro[2.3]hexan-5-yl.

In certain embodiments, R⁵ or ring A is heterocyclyl optionally substituted with one to five Z¹.

In certain embodiments. R⁵ or ring A is piperidinyl, oxabicyclo[2.2.1]heptanyl, 4,5,6,7-tetrahydropyrazolo[1,5-a]pyridinyl, tetrahydropyranyl, 2-oxopiperidyl, 4,5,6,7-tetrahydroindazolyl, 5-oxo-pyrrolidin-3-yl, oxabicyclo[2.2.2]octanyl, oxaspiro[3.3]heptanyl, or oxabicyclo[2.1.1]hexanyl; wherein each is independently optionally substituted with one to five Z¹. In certain embodiments, R⁵ or ring A is piperidinyl, oxabicyclo[2.2.1]heptanyl, 4,5,6,7-tetrahydropyrazolo[1,5-a]pyridinyl, tetrahydropyranyl, or 2-oxopiperidyl; wherein each is independently optionally substituted with one to five Z¹.

In certain embodiments, R⁵ or ring A is 5-cyano-3-fluoropyridin-2-yl, 5-chloro-3-fluoropyridin-2-yl, 1-cyclobutylpiperidin-3-yl, (1R,2R,4S)-7-oxabicyclo[2.2.1]heptan-2-yl, (1S,2R,4R)-7-oxabicyclo[2.2.1]heptan-2-yl, 1-ethylpiperidin-3-yl, 4,5,6,7-tetrahydropyrazolo[1,5-a]pyridin-5-yl, 1-methyl-6-oxo-3-piperidyl, 1-methyl-2-oxo-4-piperidyl, 2-cyclopropyltetrahydropyran-4-yl, 1-(2-methoxyethyl)-3-piperidyl, 1-ethyl-6-oxo-3-piperidyl, 4,5,6,7-tetrahydro-1H-indazol-6-yl, 1-(methoxycarbonyl)piperidin-3-yl, 1-methyl-5-oxo-pyrrolidin-3-yl, 2-oxabicyclo[2.2.2]octan-4-yl, 2-oxaspiro[3.3]heptan-6-yl, or 1-methyl-2-oxabicyclo[2.1.1]hexan-4-yl. In certain embodiments, R⁵ or ring A is 5-cyano-3-fluoropyridin-2-yl, 5-chloro-3-fluoropyridin-2-yl, 1-cyclobutylpiperidin-3-yl, (1R,2R,4S)-7-oxabicyclo[2.2.1]heptan-2-yl, (1S,2R,4R)-7-oxabicyclo[2.2.1]heptan-2-yl, 1-ethylpiperidin-3-yl, 4,5,6,7-tetrahydropyrazolo[1,5-a]pyridin-5-yl, 1-methyl-6-oxo-3-piperidyl, 1-methyl-2-oxo-4-piperidyl, 2-cyclopropyltetrahydropyran-4-yl, or 1-(2-methoxyethyl)-3-piperidyl.

In certain embodiments, R⁵ or ring A is heteroaryl optionally substituted with one to five Z¹.

In certain embodiments, R⁵ or ring A is pyrimidinyl, pyridinyl, pyridazinyl, indazolyl, or indolyl; wherein each is independently optionally substituted with one to five Z¹. In certain embodiments, R⁵ or ring A is pyrimidinyl, pyridinyl, or pyridazinyl; wherein each is independently optionally substituted with one to five Z¹.

In certain embodiments. R⁵ or ring A is 5-fluoropyrimidin-4-yl, 5-fluoropyrimidin-2-yl, 5-cyanopyrimidin-2-yl, 5-chloropyrimidin-2-yl, pyrimidin-2-yl, 5-fluoropyrimidin-4-yl, 5-cyano-3-fluoropyridin-2-yl, 5-chloro-3-fluoropyridin-2-yl, 3-fluoro-5-(trifluoromethyl)pyridin-2-yl, 6-chloropyridazin-3-yl, 3-fluoropyridin-4-yl, 3,5-difluoro-2-pyridyl, 4,5,6,7-tetrahydropyrazolo[1,5-a]pyridin-5-yl, 5-(difluoromethoxy)-2-pyridyl, 3-fluoro-5-formylpyridin-2-yl, 5-pyrazol-1-ylpyrimidin-2-yl, 5-cyanopyridin-2-yl, 1-methyl-6-oxo-3-pyridyl, 1H-indazol-6-yl, or 1H-indol-6-yl.

In certain embodiments, R⁵ or ring A is 5-fluoropyrimidin-4-yl, 5-fluoropyrimidin-2-yl, 5-cyanopyrimidin-2-yl, 5-chloropyrimidin-2-yl, pyrimidin-2-yl, 5-fluoropyrimidin-4-yl, 5-cyano-3-fluoropyridin-2-yl, 5-chloro-3-fluoropyridin-2-yl, 3-fluoro-5-(trifluoromethyl)pyridin-2-yl, 6-chloropyridazin-3-yl, 3-fluoropyridin-4-yl, 3,5-difluoro-2-pyridyl, or 4,5,6,7-tetrahydropyrazolo[1,5-a]pyridin-5-yl.

In certain embodiments, each Z^{1a} is independently halo.

In certain embodiments, each Z¹ is independently halo, hydroxy, C₁₋₆ alkyl, C₁₋₆ haloalkyl, C₃₋₁₀ cycloalkyl, or —C(O)OR¹¹.

In certain embodiments, each R¹¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl.

In certain embodiments, each R¹¹ is independently hydrogen or C₁₋₆ alkyl. In certain embodiments, each R¹¹ is hydrogen.

In certain embodiments. R¹² is C₁₋₆ alkyl, C₂₋₆ alkenyl, C₂₋₆ alkynyl, or C₁₋₆ haloalkyl. In certain embodiments, R¹² is C₁₋₆ alkyl, C₂₋₆ alkenyl, C₂₋₆ alkynyl, or C₁₋₆ haloalkyl.

In certain embodiments, each R¹³ is independently hydrogen, C₁₋₆ alkyl, C₂₋₆alkenyl, C₂₋₆ alkynyl, C₁₋₆ haloalkyl, C₃₋₁₀ cycloalkyl, heterocyclyl, aryl, or heteroaryl. In certain embodiments, each R¹³ is independently hydrogen or C₁₋₆alkyl.

In certain embodiments, provided is a compound selected from Table 1, or a pharmaceutically acceptable salt, isotopically enriched analog, prodrug, stereoisomer, or a mixture of stereoisomers thereof:

TABLE 1

| Ex | Structure |
|---|---|
| 1 | 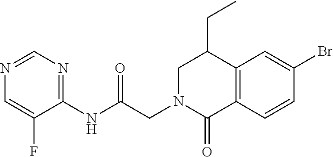 [SMILES: CCC1CN(CC(=O)Nc2ncncc2F)C(=O)c2ccc(Br)cc21] |
| 2 | 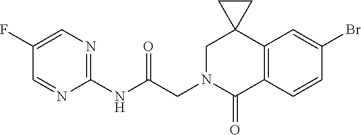 [SMILES: O=C(CN1CC2(CC2)c2cc(Br)ccc2C1=O)Nc1ncc(F)cn1] |
| 3 | 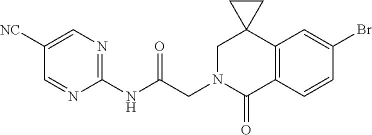 [SMILES: N#Cc1cnc(NC(=O)CN2CC3(CC3)c3cc(Br)ccc3C2=O)nc1] |
| 4 | 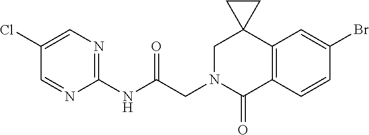 [SMILES: O=C(CN1CC2(CC2)c2cc(Br)ccc2C1=O)Nc1ncc(Cl)cn1] |
| 5 | 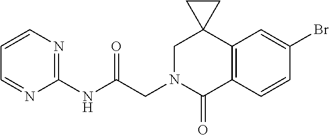 [SMILES: O=C(CN1CC2(CC2)c2cc(Br)ccc2C1=O)Nc1ncccn1] |
| 6 | 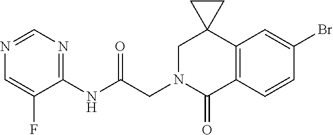 [SMILES: O=C(CN1CC2(CC2)c2cc(Br)ccc2C1=O)Nc1ncncc1F] |
| 7 | 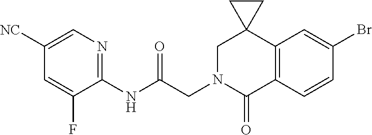 [SMILES: N#Cc1cnc(NC(=O)CN2CC3(CC3)c3cc(Br)ccc3C2=O)c(F)c1] |
| 8 | 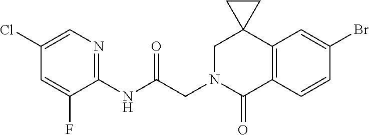 [SMILES: O=C(CN1CC2(CC2)c2cc(Br)ccc2C1=O)Nc1ncc(Cl)cc1F] |
| 9 | 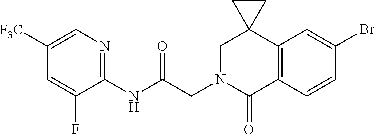 [SMILES: O=C(CN1CC2(CC2)c2cc(Br)ccc2C1=O)Nc1ncc(C(F)(F)F)cc1F] |
| 10 | 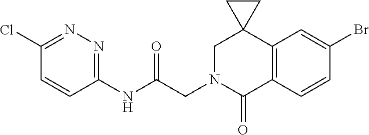 [SMILES: O=C(CN1CC2(CC2)c2cc(Br)ccc2C1=O)Nc1ccc(Cl)nn1] |
| 11 | 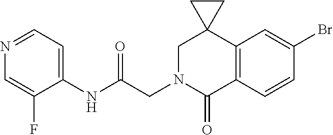 [SMILES: O=C(CN1CC2(CC2)c2cc(Br)ccc2C1=O)Nc1ccncc1F] |
| 12 | 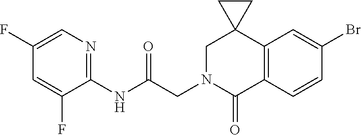 [SMILES: O=C(CN1CC2(CC2)c2cc(Br)ccc2C1=O)Nc1ncc(F)cc1F] |
| 13 | 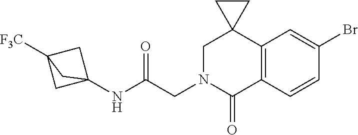 [SMILES: O=C(CN1CC2(CC2)c2cc(Br)ccc2C1=O)NC12CC(C(F)(F)F)(C1)C2] |
| 14 | 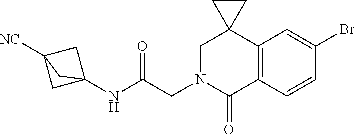 [SMILES: N#CC12CC(NC(=O)CN3CC4(CC4)c4cc(Br)ccc4C3=O)(C1)C2] |
| 15 | 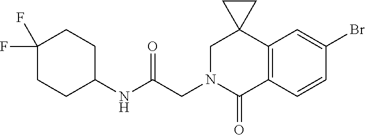 [SMILES: O=C(CN1CC2(CC2)c2cc(Br)ccc2C1=O)NC1CCC(F)(F)CC1] |
| 16 | 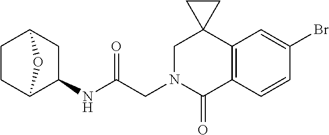 [SMILES: O=C(CN1CC2(CC2)c2cc(Br)ccc2C1=O)N[C@@H]1C[C@@H]2CC[C@H]1O2] |
| 17 | 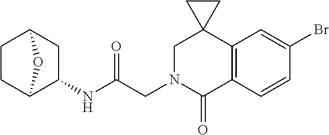 [SMILES: O=C(CN1CC2(CC2)c2cc(Br)ccc2C1=O)N[C@H]1C[C@@H]2CC[C@H]1O2] |
| 18 | 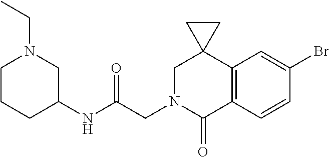 [SMILES: CCN1CCCC(NC(=O)CN2CC3(CC3)c3cc(Br)ccc3C2=O)C1] |
| 19 | 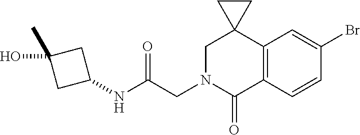 [SMILES: C[C@]1(O)C[C@@H](NC(=O)CN2CC3(CC3)c3cc(Br)ccc3C2=O)C1] |
| 20 | 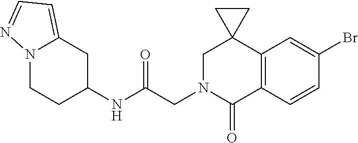 [SMILES: O=C(CN1CC2(CC2)c2cc(Br)ccc2C1=O)NC1CCn2nccc2C1] |
| 21 | 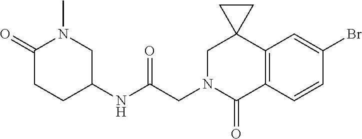 [SMILES: CN1CC(NC(=O)CN2CC3(CC3)c3cc(Br)ccc3C2=O)CCC1=O] |
| 22 | 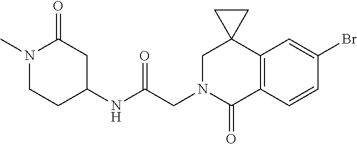 [SMILES: CN1CCC(NC(=O)CN2CC3(CC3)c3cc(Br)ccc3C2=O)CC1=O] |
| 23 | 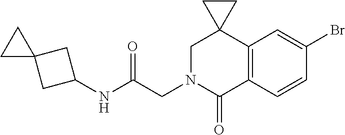 [SMILES: O=C(CN1CC2(CC2)c2cc(Br)ccc2C1=O)NC1CC2(CC2)C1] |
| 24 | 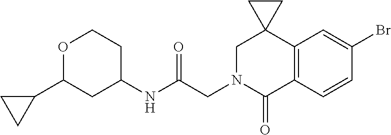 [SMILES: O=C(CN1CC2(CC2)c2cc(Br)ccc2C1=O)NC1CCOC(C2CC2)C1] |
| 25 | 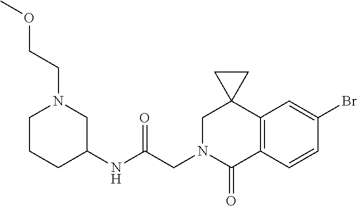 [SMILES: COCCN1CCCC(NC(=O)CN2CC3(CC3)c3cc(Br)ccc3C2=O)C1] |
| 26 | 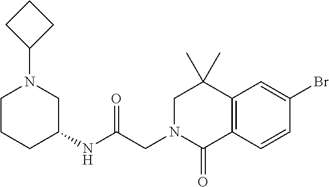 [SMILES: CC1(C)CN(CC(=O)N[C@@H]2CCCN(C3CCC3)C2)C(=O)c2ccc(Br)cc21] |
| 27 | 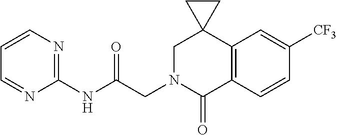 [SMILES: O=C(CN1CC2(CC2)c2cc(C(F)(F)F)ccc2C1=O)Nc1ncccn1] |
| 28 | 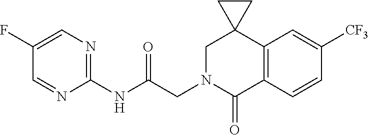 [SMILES: O=C(CN1CC2(CC2)c2cc(C(F)(F)F)ccc2C1=O)Nc1ncc(F)cn1] |
| 29 | 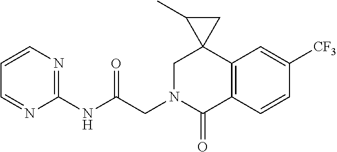 [SMILES: CC1CC12CN(CC(=O)Nc1ncccn1)C(=O)c1ccc(C(F)(F)F)cc12] |
| 30 | 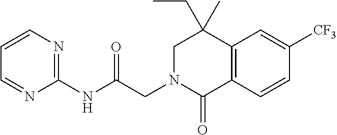 [SMILES: CCC1(C)CN(CC(=O)Nc2ncccn2)C(=O)c2ccc(C(F)(F)F)cc21] |
| 31 | 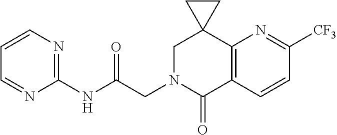 [SMILES: O=C(CN1CC2(CC2)c2nc(C(F)(F)F)ccc2C1=O)Nc1ncccn1] |
| 32 | 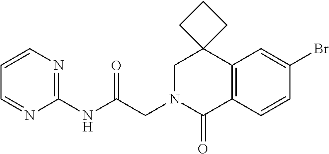 [SMILES: O=C(CN1CC2(CCC2)c2cc(Br)ccc2C1=O)Nc1ncccn1] |
| 33 | 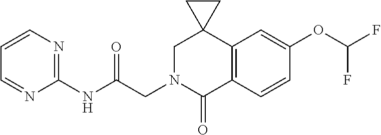 [SMILES: O=C(CN1CC2(CC2)c2cc(OC(F)F)ccc2C1=O)Nc1ncccn1] |
| 34 | 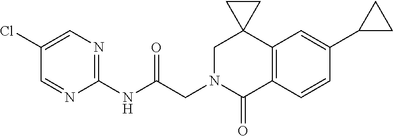 [SMILES: O=C(CN1CC2(CC2)c2cc(C3CC3)ccc2C1=O)Nc1ncc(Cl)cn1] |
| 35 | 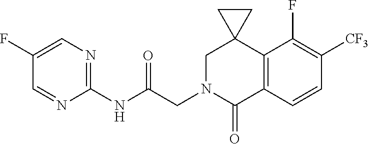 [SMILES: O=C(CN1CC2(CC2)c2c(ccc(C(F)(F)F)c2F)C1=O)Nc1ncc(F)cn1] |
| 36 | 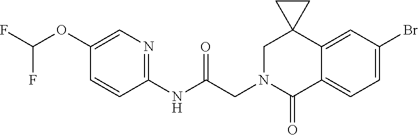 [SMILES: O=C(CN1CC2(CC2)c2cc(Br)ccc2C1=O)Nc1ccc(OC(F)F)cn1] |
| 37 | 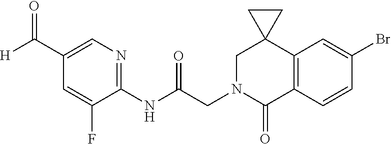 [SMILES: O=Cc1cnc(NC(=O)CN2CC3(CC3)c3cc(Br)ccc3C2=O)c(F)c1] |
| 38 | 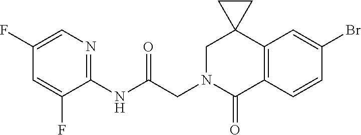 [SMILES: O=C(CN1CC2(CC2)c2cc(Br)ccc2C1=O)Nc1ncc(F)cc1F] |
| 39 | 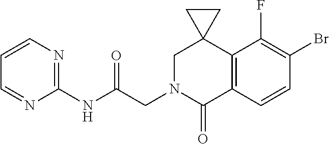 [SMILES: O=C(CN1CC2(CC2)c2c(ccc(Br)c2F)C1=O)Nc1ncccn1] |
| 40 | 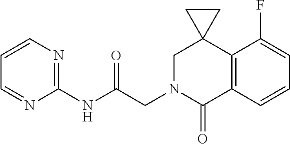 [SMILES: O=C(CN1CC2(CC2)c2c(F)cccc2C1=O)Nc1ncccn1] |
| 41 | 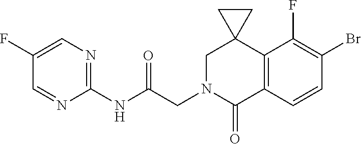 [SMILES: O=C(CN1CC2(CC2)c2c(ccc(Br)c2F)C1=O)Nc1ncc(F)cn1] |
| 42 | 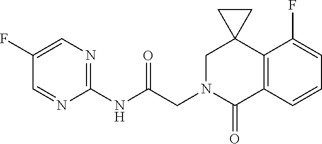 [SMILES: O=C(CN1CC2(CC2)c2c(F)cccc2C1=O)Nc1ncc(F)cn1] |
| 43 | 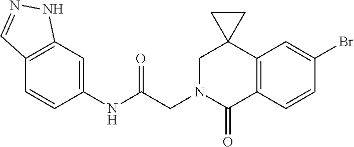 [SMILES: O=C(CN1CC2(CC2)c2cc(Br)ccc2C1=O)Nc1ccc2cn[nH]c2c1] |
| 44 | 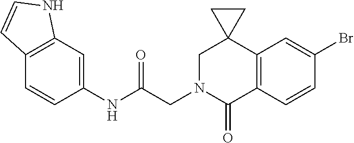 [SMILES: O=C(CN1CC2(CC2)c2cc(Br)ccc2C1=O)Nc1ccc2cc[nH]c2c1] |
| 45 | 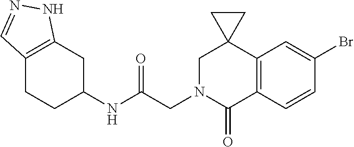 [SMILES: O=C(CN1CC2(CC2)c2cc(Br)ccc2C1=O)NC1CCc2cn[nH]c2C1] |
| 46 | 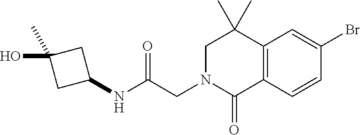 [SMILES: CC1(C)CN(CC(=O)N[C@H]2C[C@@](C)(O)C2)C(=O)c2ccc(Br)cc21] |
| 47 | 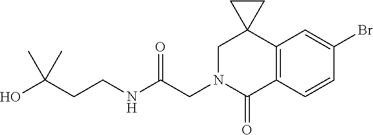 [SMILES: CC(C)(O)CCNC(=O)CN1CC2(CC2)c2cc(Br)ccc2C1=O] |
| 48 | 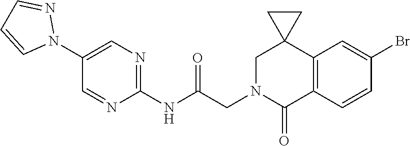 [SMILES: O=C(CN1CC2(CC2)c2cc(Br)ccc2C1=O)Nc1ncc(-n2cccn2)cn1] |
| 49 | 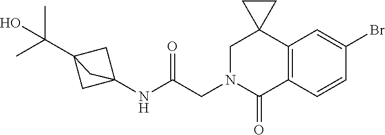 [SMILES: CC(C)(O)C12CC(NC(=O)CN3CC4(CC4)c4cc(Br)ccc4C3=O)(C1)C2] |
| 50 | 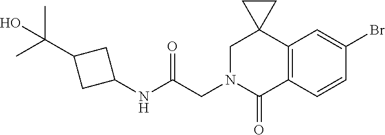 [SMILES: CC(C)(O)C1CC(NC(=O)CN2CC3(CC3)c3cc(Br)ccc3C2=O)C1] |
| 51 | 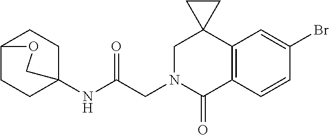 [SMILES: O=C(CN1CC2(CC2)c2cc(Br)ccc2C1=O)NC12CCC(CC1)OC2] |
| 52 | 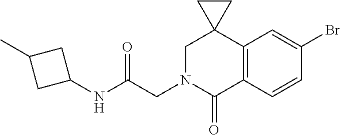 [SMILES: CC1CC(NC(=O)CN2CC3(CC3)c3cc(Br)ccc3C2=O)C1] |
| 53 | 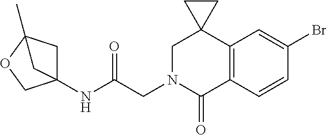 [SMILES: CC12CC(NC(=O)CN3CC4(CC4)c4cc(Br)ccc4C3=O)(CO1)C2] |
| 54 | 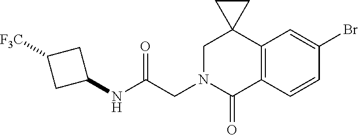 |
| 55 | 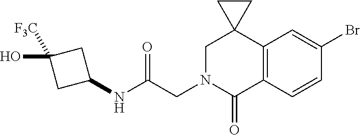 |
| 56 | 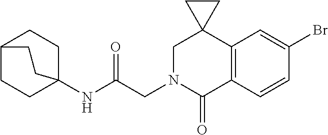 [SMILES: O=C(CN1CC2(CC2)c2cc(Br)ccc2C1=O)NC12CCC(CC1)CC2] |
| 57 | 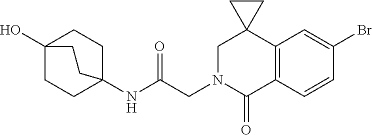 [SMILES: O=C(CN1CC2(CC2)c2cc(Br)ccc2C1=O)NC12CCC(O)(CC1)CC2] |
| 58 | 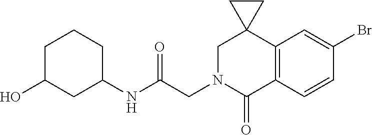 [SMILES: O=C(CN1CC2(CC2)c2cc(Br)ccc2C1=O)NC1CCCC(O)C1] |
| 59 | 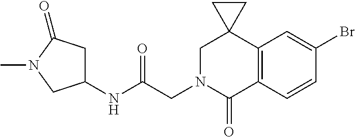 [SMILES: CN1CC(NC(=O)CN2CC3(CC3)c3cc(Br)ccc3C2=O)CC1=O] |
| 60 | 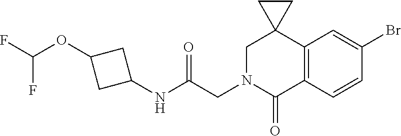 [SMILES: O=C(CN1CC2(CC2)c2cc(Br)ccc2C1=O)NC1CC(OC(F)F)C1] |
| 61 | 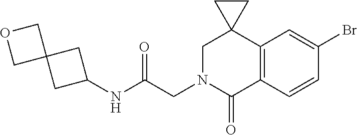 [SMILES: O=C(CN1CC2(CC2)c2cc(Br)ccc2C1=O)NC1CC2(COC2)C1] |
| 62 | 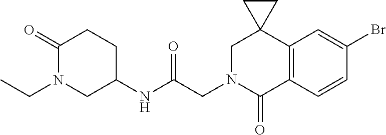 [SMILES: CCN1CC(NC(=O)CN2CC3(CC3)c3cc(Br)ccc3C2=O)CCC1=O] |
| 63 | 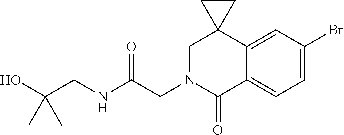 [SMILES: CC(C)(O)CNC(=O)CN1CC2(CC2)c2cc(Br)ccc2C1=O] |
| 64 | 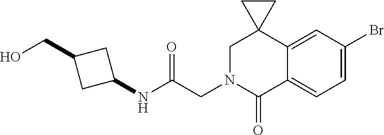 |
| 65 | 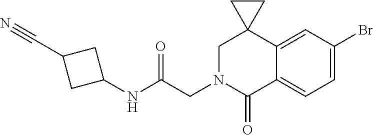 [SMILES: N#CC1CC(NC(=O)CN2CC3(CC3)c3cc(Br)ccc3C2=O)C1] |
| 66 | 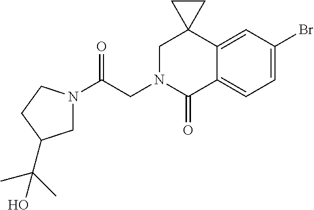 [SMILES: CC(C)(O)C1CCN(C(=O)CN2CC3(CC3)c3cc(Br)ccc3C2=O)C1] |
| 67 | 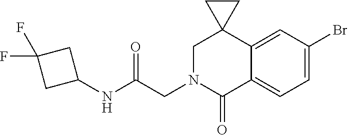 [SMILES: O=C(CN1CC2(CC2)c2cc(Br)ccc2C1=O)NC1CC(F)(F)C1] |
| 68 | 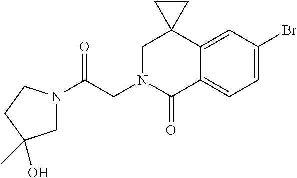 [SMILES: CC1(O)CCN(C(=O)CN2CC3(CC3)c3cc(Br)ccc3C2=O)C1] |
| 69 | 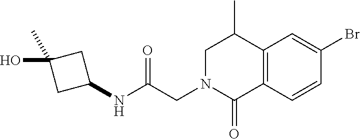 |
| 70 | 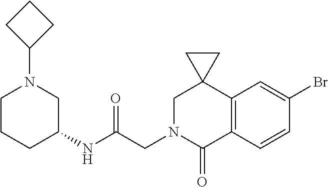 [SMILES: O=C(CN1CC2(CC2)c2cc(Br)ccc2C1=O)N[C@@H]1CCCN(C2CCC2)C1] |
| 71 | 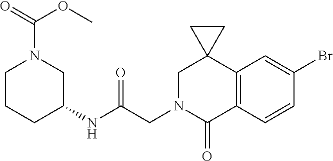 [SMILES: COC(=O)N1CCC[C@@H](NC(=O)CN2CC3(CC3)c3cc(Br)ccc3C2=O)C1] |
| 72 | 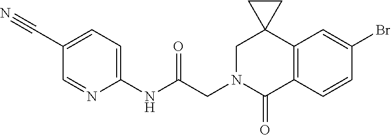 [SMILES: N#Cc1ccc(NC(=O)CN2CC3(CC3)c3cc(Br)ccc3C2=O)nc1] |
| 73 | 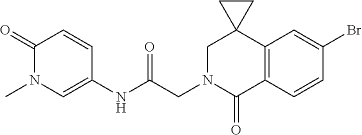 [SMILES: Cn1cc(NC(=O)CN2CC3(CC3)c3cc(Br)ccc3C2=O)ccc1=O] |
| 74 | 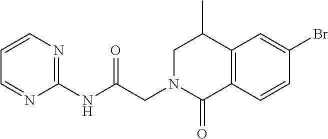 [SMILES: CC1CN(CC(=O)Nc2ncccn2)C(=O)c2ccc(Br)cc21] |
| 75 | 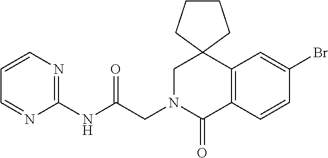 [SMILES: O=C(CN1CC2(CCCC2)c2cc(Br)ccc2C1=O)Nc1ncccn1] |
| 76 | 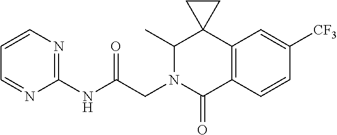 [SMILES: CC1N(CC(=O)Nc2ncccn2)C(=O)c2ccc(C(F)(F)F)cc2C12CC2] |
| 77 | 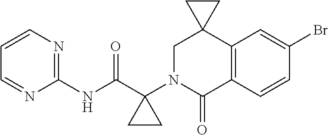 [SMILES: O=C1c2ccc(Br)cc2C2(CC2)CN1C1(C(=O)Nc2ncccn2)CC1] |
| 78 | 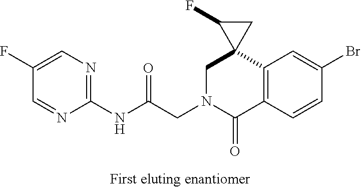 |
| 79 | 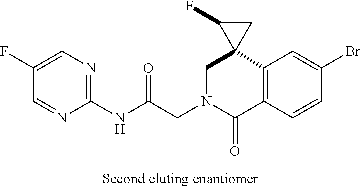 |
| 80 | 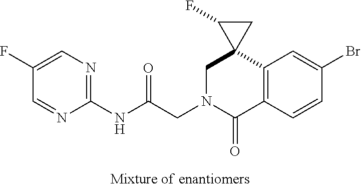 |
| 81 | 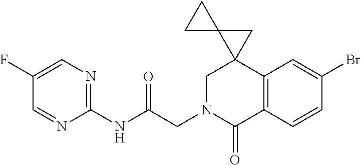 [SMILES: O=C(CN1CC2(CC23CC3)c2cc(Br)ccc2C1=O)Nc1ncc(F)cn1] |
| 82 | 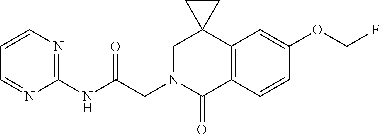 [SMILES: O=C(CN1CC2(CC2)c2cc(OCF)ccc2C1=O)Nc1ncccn1] |
| 83 | 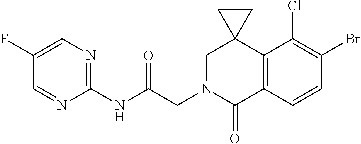 [SMILES: O=C(CN1CC2(CC2)c2c(ccc(Br)c2Cl)C1=O)Nc1ncc(F)cn1] |
| 84 | 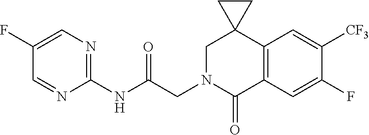 [SMILES: O=C(CN1CC2(CC2)c2cc(C(F)(F)F)c(F)cc2C1=O)Nc1ncc(F)cn1] |
| 85 | 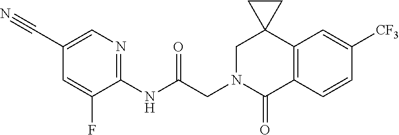 [SMILES: N#Cc1cnc(NC(=O)CN2CC3(CC3)c3cc(C(F)(F)F)ccc3C2=O)c(F)c1] |
| 86 | 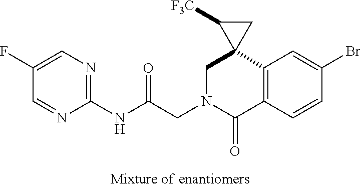 |
| 87 | 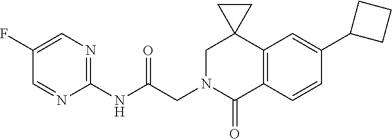 [SMILES: O=C(CN1CC2(CC2)c2cc(C3CCC3)ccc2C1=O)Nc1ncc(F)cn1] |
| 88 | 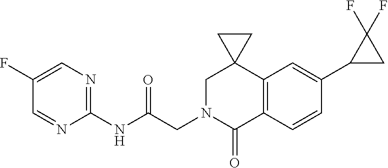 [SMILES: O=C(CN1CC2(CC2)c2cc(C3CC3(F)F)ccc2C1=O)Nc1ncc(F)cn1] |
| 89 | 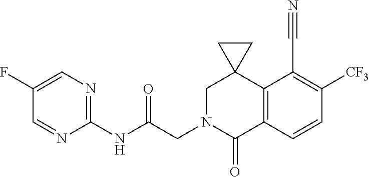 [SMILES: N#Cc1c(C(F)(F)F)ccc2c1C1(CC1)CN(CC(=O)Nc1ncc(F)cn1)C2=O] |
| 90 | 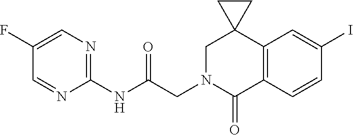 [SMILES: O=C(CN1CC2(CC2)c2cc(I)ccc2C1=O)Nc1ncc(F)cn1] |
| 91 | 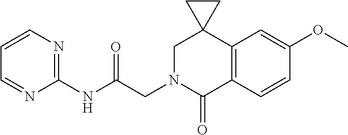 [SMILES: COc1ccc2c(c1)C1(CC1)CN(CC(=O)Nc1ncccn1)C2=O] |
| 92 | 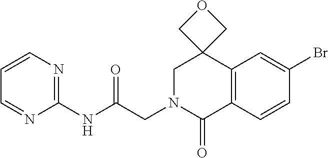 [SMILES: O=C(CN1CC2(COC2)c2cc(Br)ccc2C1=O)Nc1ncccn1] |
| 93 | 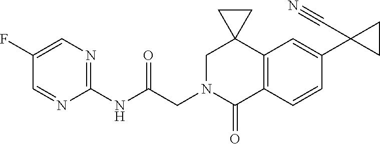 [SMILES: N#CC1(c2ccc3c(c2)C2(CC2)CN(CC(=O)Nc2ncc(F)cn2)C3=O)CC1] |
| 94 | 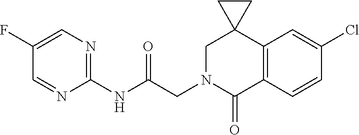 [SMILES: O=C(CN1CC2(CC2)c2cc(Cl)ccc2C1=O)Nc1ncc(F)cn1] |
| 95 | 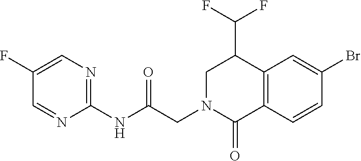 [SMILES: O=C(CN1CC(C(F)F)c2cc(Br)ccc2C1=O)Nc1ncc(F)cn1] |
| 96 | 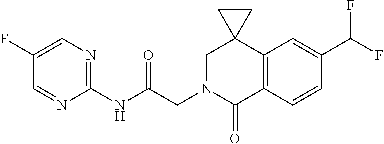 [SMILES: O=C(CN1CC2(CC2)c2cc(C(F)F)ccc2C1=O)Nc1ncc(F)cn1] |
| 97 | 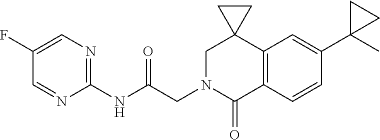 [SMILES: CC1(c2ccc3c(c2)C2(CC2)CN(CC(=O)Nc2ncc(F)cn2)C3=O)CC1] |
| 98 | 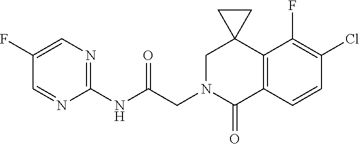 [SMILES: O=C(CN1CC2(CC2)c2c(ccc(Cl)c2F)C1=O)Nc1ncc(F)cn1] |
| 99 | 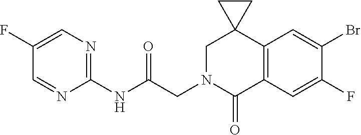 [SMILES: O=C(CN1CC2(CC2)c2cc(Br)c(F)cc2C1=O)Nc1ncc(F)cn1] |
| 100 | 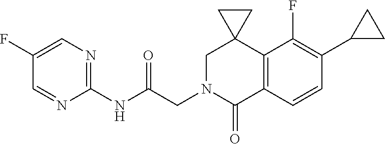 [SMILES: O=C(CN1CC2(CC2)c2c(ccc(C3CC3)c2F)C1=O)Nc1ncc(F)cn1] |
| 101 | 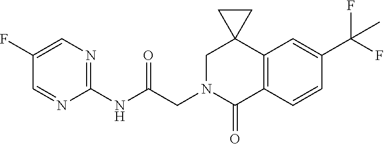 [SMILES: CC(F)(F)c1ccc2c(c1)C1(CC1)CN(CC(=O)Nc1ncc(F)cn1)C2=O] |
| 102 | 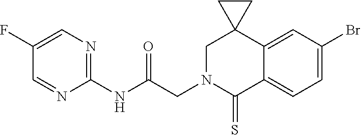 [SMILES: O=C(CN1CC2(CC2)c2cc(Br)ccc2C1=S)Nc1ncc(F)cn1] |
| 103 | 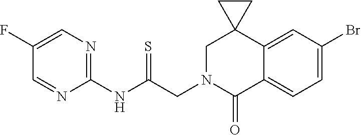 [SMILES: O=C1c2ccc(Br)cc2C2(CC2)CN1CC(=S)Nc1ncc(F)cn1] |
| 104 | 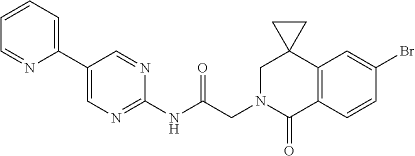 [SMILES: O=C(CN1CC2(CC2)c2cc(Br)ccc2C1=O)Nc1ncc(-c2ccccn2)cn1] |
| 105 | 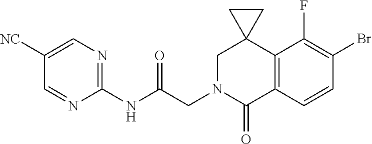 [SMILES: N#Cc1cnc(NC(=O)CN2CC3(CC3)c3c(ccc(Br)c3F)C2=O)nc1] |
| 106 | 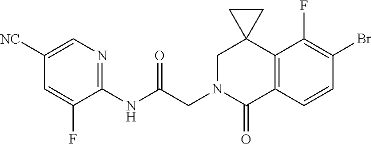 [SMILES: N#Cc1cnc(NC(=O)CN2CC3(CC3)c3c(ccc(Br)c3F)C2=O)c(F)c1] |
| 107 | 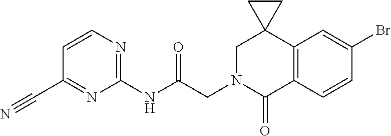 [SMILES: N#Cc1ccnc(NC(=O)CN2CC3(CC3)c3cc(Br)ccc3C2=O)n1] |
| 108 | 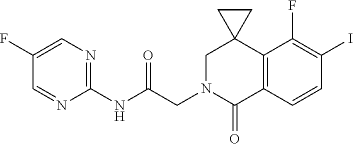 [SMILES: O=C(CN1CC2(CC2)c2c(ccc(I)c2F)C1=O)Nc1ncc(F)cn1] |
| 109 | 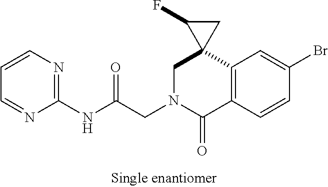 |
| 110 | 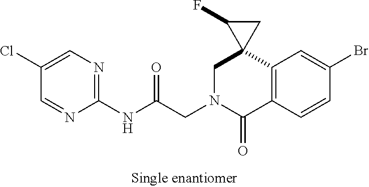 |
| 111 | 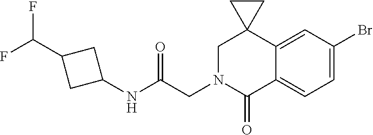 [SMILES: O=C(CN1CC2(CC2)c2cc(Br)ccc2C1=O)NC1CC(C(F)F)C1] |
| 112 | 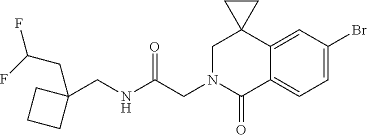 [SMILES: O=C(CN1CC2(CC2)c2cc(Br)ccc2C1=O)NCC1(CC(F)F)CCC1] |
| 113 | 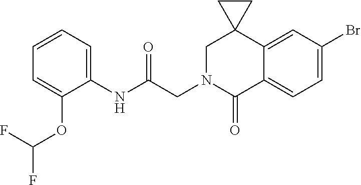 [SMILES: O=C(CN1CC2(CC2)c2cc(Br)ccc2C1=O)Nc1ccccc1OC(F)F] |
| 114 | 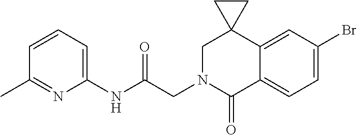 [SMILES: Cc1cccc(NC(=O)CN2CC3(CC3)c3cc(Br)ccc3C2=O)n1] |
| 115 | 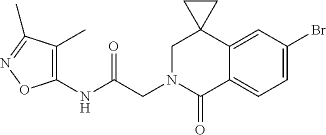 [SMILES: Cc1noc(NC(=O)CN2CC3(CC3)c3cc(Br)ccc3C2=O)c1C] |
| 116 | 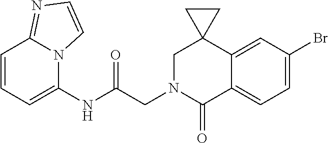 [SMILES: O=C(CN1CC2(CC2)c2cc(Br)ccc2C1=O)Nc1cccc2nccn12] |
| 117 | 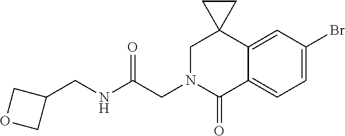 [SMILES: O=C(CN1CC2(CC2)c2cc(Br)ccc2C1=O)NCC1COC1] |
| 118 | 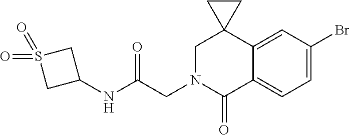 [SMILES: O=C(CN1CC2(CC2)c2cc(Br)ccc2C1=O)NC1CS(=O)(=O)C1] |
| 119 | 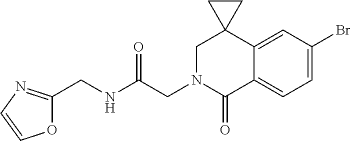 [SMILES: O=C(CN1CC2(CC2)c2cc(Br)ccc2C1=O)NCc1ncco1] |
| 120 | 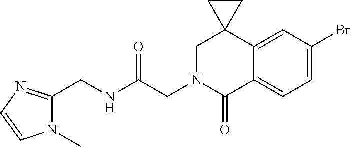 [SMILES: Cn1ccnc1CNC(=O)CN1CC2(CC2)c2cc(Br)ccc2C1=O] |
| 121 | 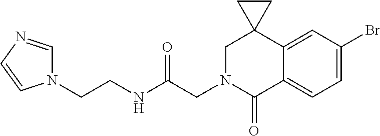 [SMILES: O=C(CN1CC2(CC2)c2cc(Br)ccc2C1=O)NCCn1ccnc1] |
| 122 | 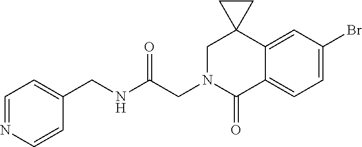 [SMILES: O=C(CN1CC2(CC2)c2cc(Br)ccc2C1=O)NCc1ccncc1] |
| 123 | 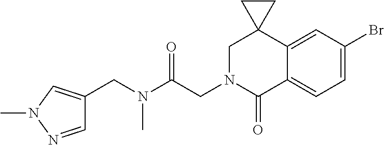 [SMILES: CN(Cc1cnn(C)c1)C(=O)CN1CC2(CC2)c2cc(Br)ccc2C1=O] |
| 124 | 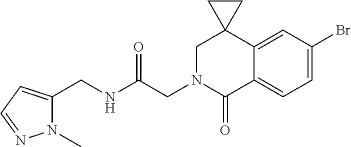 [SMILES: Cn1nccc1CNC(=O)CN1CC2(CC2)c2cc(Br)ccc2C1=O] |
| 125 | 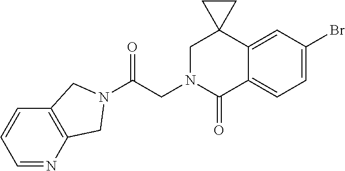 [SMILES: O=C(CN1CC2(CC2)c2cc(Br)ccc2C1=O)N1Cc2cccnc2C1] |
| 126 | 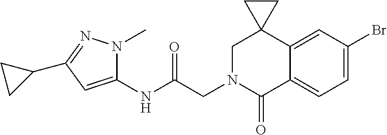 [SMILES: Cn1nc(C2CC2)cc1NC(=O)CN1CC2(CC2)c2cc(Br)ccc2C1=O] |
| 127 | 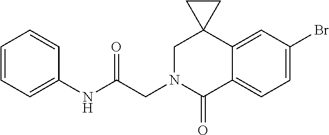 [SMILES: O=C(CN1CC2(CC2)c2cc(Br)ccc2C1=O)Nc1ccccc1] |
| 128 | 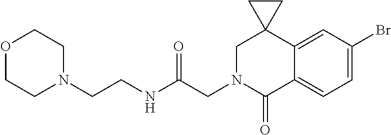 [SMILES: O=C(CN1CC2(CC2)c2cc(Br)ccc2C1=O)NCCN1CCOCC1] |
| 129 | 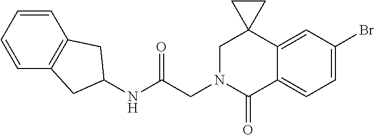 [SMILES: O=C(CN1CC2(CC2)c2cc(Br)ccc2C1=O)NC1Cc2ccccc2C1] |
| 130 | 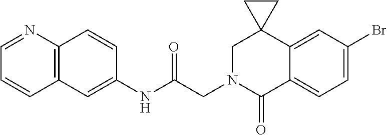 [SMILES: O=C(CN1CC2(CC2)c2cc(Br)ccc2C1=O)Nc1ccc2ncccc2c1] |
| 131 | 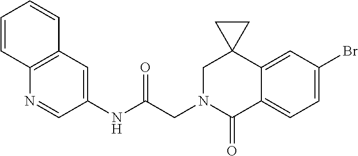 [SMILES: O=C(CN1CC2(CC2)c2cc(Br)ccc2C1=O)Nc1cnc2ccccc2c1] |
| 132 | 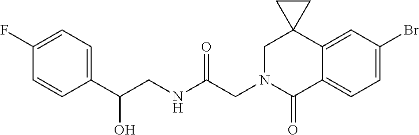 [SMILES: O=C(CN1CC2(CC2)c2cc(Br)ccc2C1=O)NCC(O)c1ccc(F)cc1] |
| 133 | 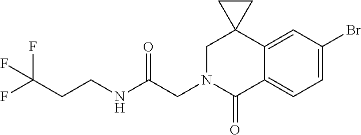 [SMILES: O=C(CN1CC2(CC2)c2cc(Br)ccc2C1=O)NCCC(F)(F)F] |
| 134 | 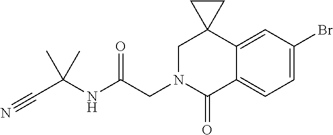 [SMILES: CC(C)(C#N)NC(=O)CN1CC2(CC2)c2cc(Br)ccc2C1=O] |
| 135 | 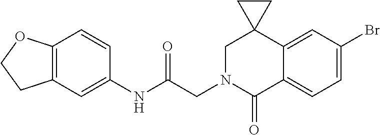 [SMILES: O=C(CN1CC2(CC2)c2cc(Br)ccc2C1=O)Nc1ccc2c(c1)CCO2] |
| 136 | 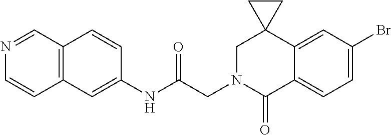 [SMILES: O=C(CN1CC2(CC2)c2cc(Br)ccc2C1=O)Nc1ccc2cnccc2c1] |
| 137 | 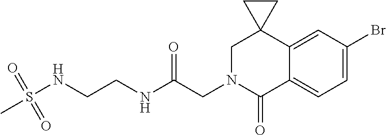 [SMILES: CS(=O)(=O)NCCNC(=O)CN1CC2(CC2)c2cc(Br)ccc2C1=O] |
| 138 | 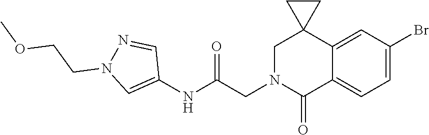 [SMILES: COCCn1cc(NC(=O)CN2CC3(CC3)c3cc(Br)ccc3C2=O)cn1] |
| 139 | 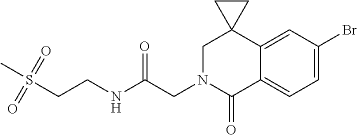 [SMILES: CS(=O)(=O)CCNC(=O)CN1CC2(CC2)c2cc(Br)ccc2C1=O] |
| 140 | 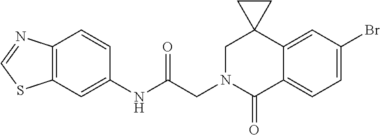 [SMILES: O=C(CN1CC2(CC2)c2cc(Br)ccc2C1=O)Nc1ccc2ncsc2c1] |
| 141 | 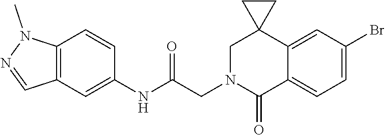 [SMILES: Cn1ncc2cc(NC(=O)CN3CC4(CC4)c4cc(Br)ccc4C3=O)ccc21] |
| 142 | 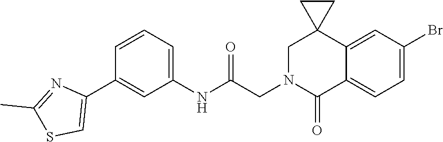 [SMILES: Cc1nc(-c2cccc(NC(=O)CN3CC4(CC4)c4cc(Br)ccc4C3=O)c2)cs1] |
| 143 | 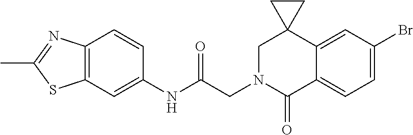 [SMILES: Cc1nc2ccc(NC(=O)CN3CC4(CC4)c4cc(Br)ccc4C3=O)cc2s1] |
| 144 | 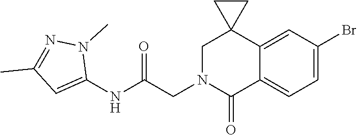 [SMILES: Cc1cc(NC(=O)CN2CC3(CC3)c3cc(Br)ccc3C2=O)n(C)n1] |
| 145 | 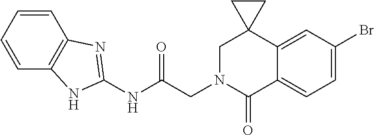 [SMILES: O=C(CN1CC2(CC2)c2cc(Br)ccc2C1=O)Nc1nc2ccccc2[nH]1] |
| 146 | 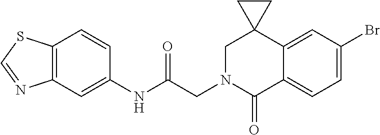 [SMILES: O=C(CN1CC2(CC2)c2cc(Br)ccc2C1=O)Nc1ccc2scnc2c1] |
| 147 | 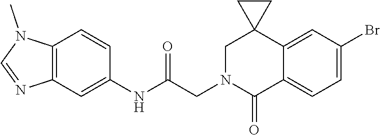 [SMILES: Cn1cnc2cc(NC(=O)CN3CC4(CC4)c4cc(Br)ccc4C3=O)ccc21] |
| 148 | 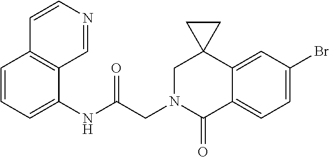 [SMILES: O=C(CN1CC2(CC2)c2cc(Br)ccc2C1=O)Nc1cccc2ccncc12] |
| 149 | 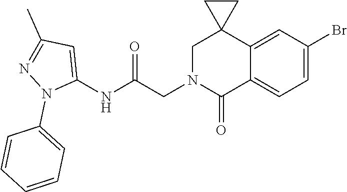 [SMILES: Cc1cc(NC(=O)CN2CC3(CC3)c3cc(Br)ccc3C2=O)n(-c2ccccc2)n1] |
| 150 | 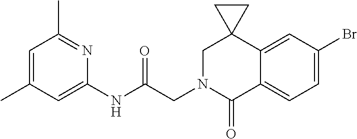 [SMILES: Cc1cc(C)nc(NC(=O)CN2CC3(CC3)c3cc(Br)ccc3C2=O)c1] |
| 151 | 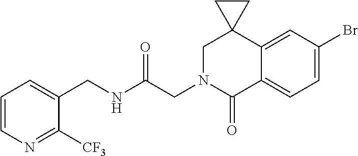 [SMILES: O=C(CN1CC2(CC2)c2cc(Br)ccc2C1=O)NCc1cccnc1C(F)(F)F] |
| 152 | 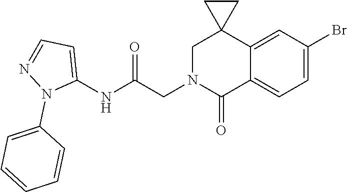 [SMILES: O=C(CN1CC2(CC2)c2cc(Br)ccc2C1=O)Nc1ccnn1-c1ccccc1] |
| 153 | 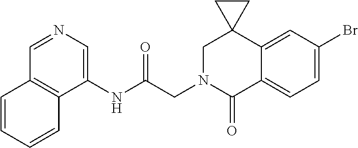 [SMILES: O=C(CN1CC2(CC2)c2cc(Br)ccc2C1=O)Nc1cncc2ccccc12] |
| 154 | 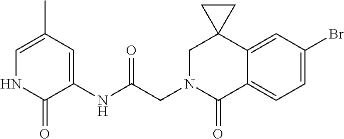 [SMILES: Cc1c[nH]c(=O)c(NC(=O)CN2CC3(CC3)c3cc(Br)ccc3C2=O)c1] |
| 155 | 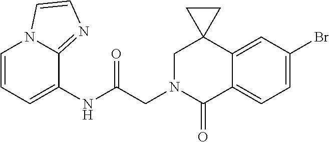 [SMILES: O=C(CN1CC2(CC2)c2cc(Br)ccc2C1=O)Nc1cccn2ccnc12] |
| 156 | 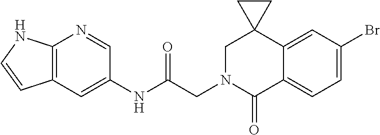 [SMILES: O=C(CN1CC2(CC2)c2cc(Br)ccc2C1=O)Nc1cnc2[nH]ccc2c1] |
| 157 | 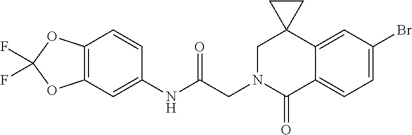 [SMILES: O=C(CN1CC2(CC2)c2cc(Br)ccc2C1=O)Nc1ccc2c(c1)OC(F)(F)O2] |
| 158 | 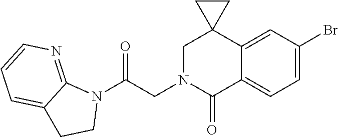 [SMILES: O=C1c2ccc(Br)cc2C2(CC2)CN1CC(=O)N1CCc2cccnc21] |
| 159 | 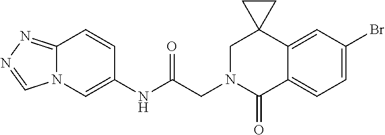 [SMILES: O=C(CN1CC2(CC2)c2cc(Br)ccc2C1=O)Nc1ccc2nncn2c1] |
| 160 | 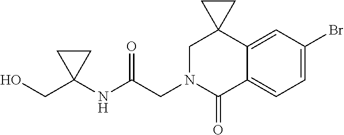 [SMILES: O=C(CN1CC2(CC2)c2cc(Br)ccc2C1=O)NC1(CO)CC1] |
| 161 | 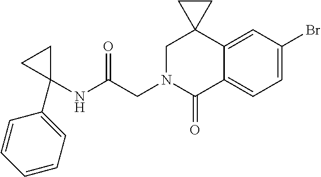 [SMILES: O=C(CN1CC2(CC2)c2cc(Br)ccc2C1=O)NC1(c2ccccc2)CC1] |
| 162 | 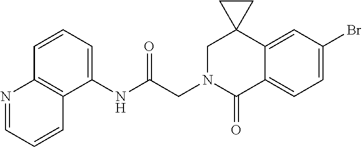 [SMILES: O=C(CN1CC2(CC2)c2cc(Br)ccc2C1=O)Nc1cccc2ncccc12] |
| 163 | 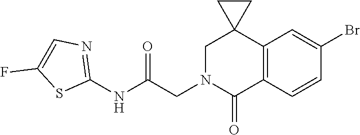 [SMILES: O=C(CN1CC2(CC2)c2cc(Br)ccc2C1=O)Nc1ncc(F)s1] |
| 164 | 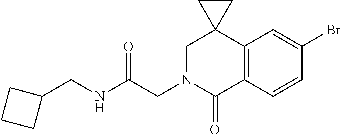 [SMILES: O=C(CN1CC2(CC2)c2cc(Br)ccc2C1=O)NCC1CCC1] |
| 165 | 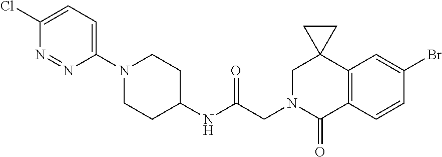 [SMILES: O=C(CN1CC2(CC2)c2cc(Br)ccc2C1=O)NC1CCN(c2ccc(Cl)nn2)CC1] |
| 166 | 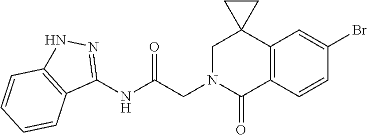 [SMILES: O=C(CN1CC2(CC2)c2cc(Br)ccc2C1=O)Nc1n[nH]c2ccccc12] |
| 167 | 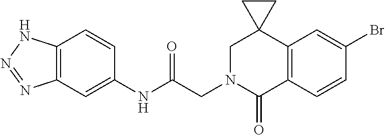 |
| 168 | 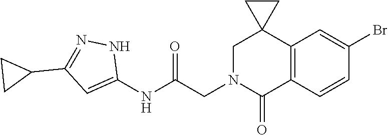 [SMILES: O=C(CN1CC2(CC2)c2cc(Br)ccc2C1=O)Nc1cc(C2CC2)n[nH]1] |
| 169 | 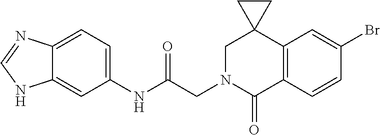 [SMILES: O=C(CN1CC2(CC2)c2cc(Br)ccc2C1=O)Nc1ccc2nc[nH]c2c1] |
| 170 | 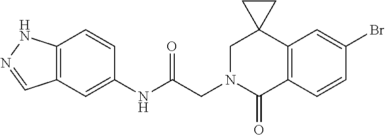 [SMILES: O=C(CN1CC2(CC2)c2cc(Br)ccc2C1=O)Nc1ccc2[nH]ncc2c1] |
| 171 | 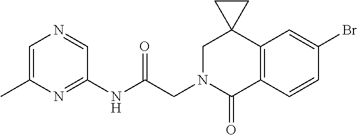 [SMILES: Cc1cncc(NC(=O)CN2CC3(CC3)c3cc(Br)ccc3C2=O)n1] |
| 172 | 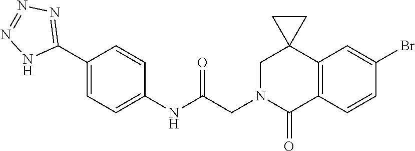 [SMILES: O=C(CN1CC2(CC2)c2cc(Br)ccc2C1=O)Nc1ccc(-c2nnn[nH]2)cc1] |
| 173 | 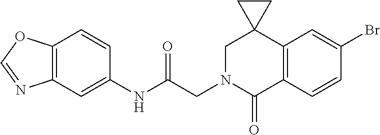 [SMILES: O=C(CN1CC2(CC2)c2cc(Br)ccc2C1=O)Nc1ccc2ocnc2c1] |
| 174 | 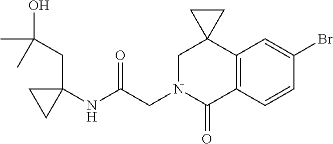 [SMILES: CC(C)(O)CC1(NC(=O)CN2CC3(CC3)c3cc(Br)ccc3C2=O)CC1] |
| 175 | 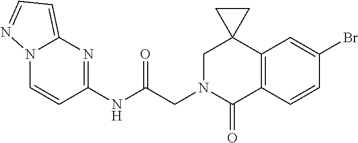 [SMILES: O=C(CN1CC2(CC2)c2cc(Br)ccc2C1=O)Nc1ccn2nccc2n1] |
| 176 | 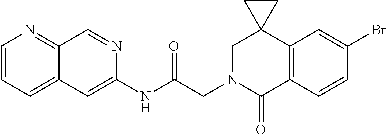 [SMILES: O=C(CN1CC2(CC2)c2cc(Br)ccc2C1=O)Nc1cc2cccnc2cn1] |
| 177 | 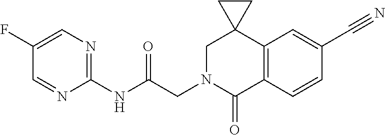 [SMILES: N#Cc1ccc2c(c1)C1(CC1)CN(CC(=O)Nc1ncc(F)cn1)C2=O] |
| 178 | 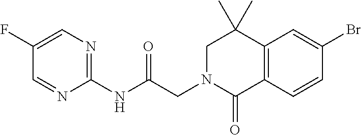 [SMILES: CC1(C)CN(CC(=O)Nc2ncc(F)cn2)C(=O)c2ccc(Br)cc21] |
| 179 | 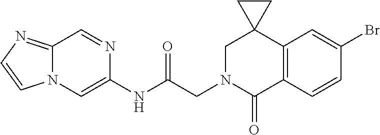 [SMILES: O=C(CN1CC2(CC2)c2cc(Br)ccc2C1=O)Nc1cn2ccnc2cn1] |
| 180 | 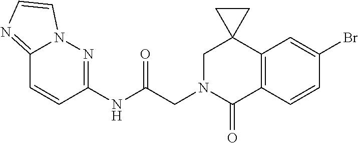 [SMILES: O=C(CN1CC2(CC2)c2cc(Br)ccc2C1=O)Nc1ccc2nccn2n1] |
| 181 | 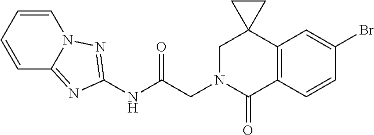 [SMILES: O=C(CN1CC2(CC2)c2cc(Br)ccc2C1=O)Nc1nc2ccccn2n1] |
| 182 | 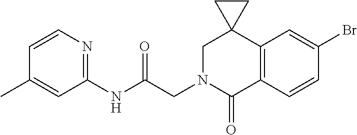 [SMILES: Cc1ccnc(NC(=O)CN2CC3(CC3)c3cc(Br)ccc3C2=O)c1] |
| 183 | 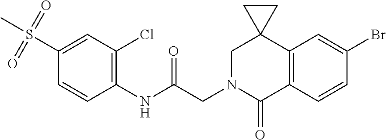 [SMILES: CS(=O)(=O)c1ccc(NC(=O)CN2CC3(CC3)c3cc(Br)ccc3C2=O)c(Cl)c1] |
| 184 | 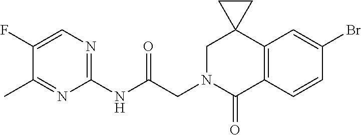 [SMILES: Cc1nc(NC(=O)CN2CC3(CC3)c3cc(Br)ccc3C2=O)ncc1F] |
| 185 | 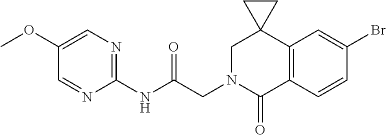 [SMILES: COc1cnc(NC(=O)CN2CC3(CC3)c3cc(Br)ccc3C2=O)nc1] |
| 186 | 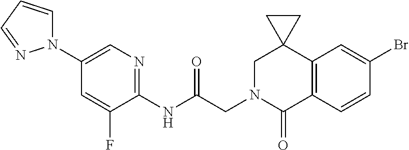 [SMILES: O=C(CN1CC2(CC2)c2cc(Br)ccc2C1=O)Nc1ncc(-n2cccn2)cc1F] |
| 187 | 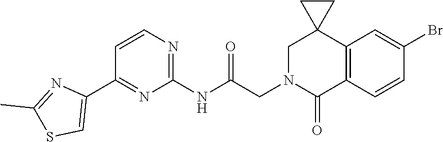 [SMILES: Cc1nc(-c2ccnc(NC(=O)CN3CC4(CC4)c4cc(Br)ccc4C3=O)n2)cs1] |
| 188 | 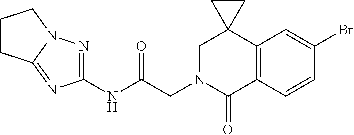 [SMILES: O=C(CN1CC2(CC2)c2cc(Br)ccc2C1=O)Nc1nc2n(n1)CCC2] |
| 189 | 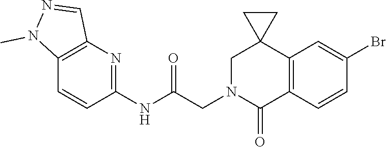 [SMILES: Cn1ncc2nc(NC(=O)CN3CC4(CC4)c4cc(Br)ccc4C3=O)ccc21] |
| 190 | 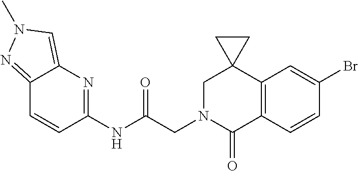 [SMILES: Cn1cc2nc(NC(=O)CN3CC4(CC4)c4cc(Br)ccc4C3=O)ccc2n1] |
| 191 | 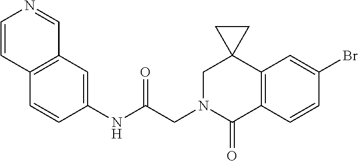 [SMILES: O=C(CN1CC2(CC2)c2cc(Br)ccc2C1=O)Nc1ccc2ccncc2c1] |
| 192 | 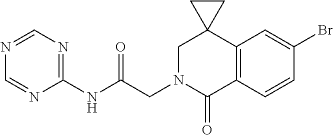 [SMILES: O=C(CN1CC2(CC2)c2cc(Br)ccc2C1=O)Nc1ncncn1] |
| 193 | 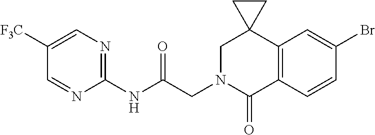 [SMILES: O=C(CN1CC2(CC2)c2cc(Br)ccc2C1=O)Nc1ncc(C(F)(F)F)cn1] |
| 194 | 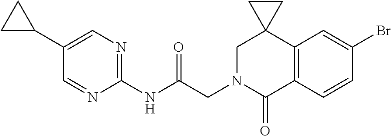 [SMILES: O=C(CN1CC2(CC2)c2cc(Br)ccc2C1=O)Nc1ncc(C2CC2)cn1] |
| 195 | 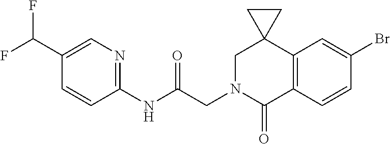 [SMILES: O=C(CN1CC2(CC2)c2cc(Br)ccc2C1=O)Nc1ccc(C(F)F)cn1] |
| 196 | 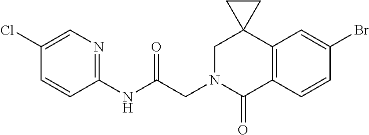 [SMILES: O=C(CN1CC2(CC2)c2cc(Br)ccc2C1=O)Nc1ccc(Cl)cn1] |
| 197 | 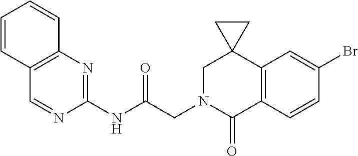 [SMILES: O=C(CN1CC2(CC2)c2cc(Br)ccc2C1=O)Nc1ncc2ccccc2n1] |
| 198 | 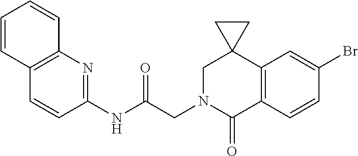 [SMILES: O=C(CN1CC2(CC2)c2cc(Br)ccc2C1=O)Nc1ccc2ccccc2n1] |
| 199 | 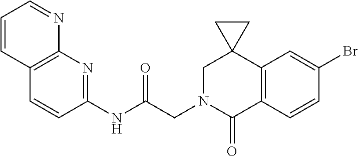 [SMILES: O=C(CN1CC2(CC2)c2cc(Br)ccc2C1=O)Nc1ccc2cccnc2n1] |
| 200 | 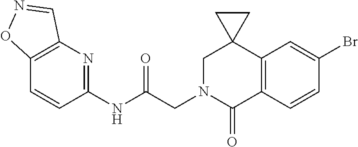 [SMILES: O=C(CN1CC2(CC2)c2cc(Br)ccc2C1=O)Nc1ccc2oncc2n1] |
| 201 | 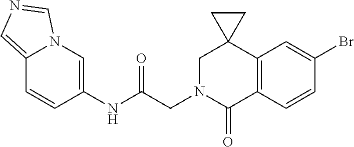 [SMILES: O=C(CN1CC2(CC2)c2cc(Br)ccc2C1=O)Nc1ccc2cncn2c1] |
| 202 | 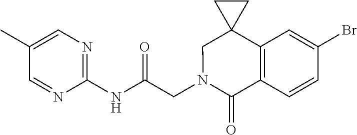 [SMILES: Cc1cnc(NC(=O)CN2CC3(CC3)c3cc(Br)ccc3C2=O)nc1] |
| 203 | 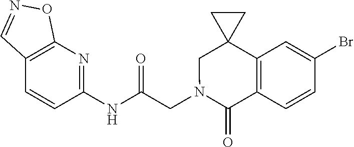 [SMILES: O=C(CN1CC2(CC2)c2cc(Br)ccc2C1=O)Nc1ccc2cnoc2n1] |
| 204 | 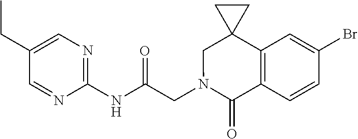 [SMILES: CCc1cnc(NC(=O)CN2CC3(CC3)c3cc(Br)ccc3C2=O)nc1] |
| 205 | 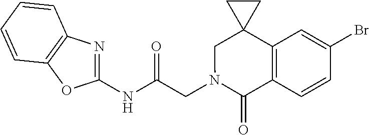 [SMILES: O=C(CN1CC2(CC2)c2cc(Br)ccc2C1=O)Nc1nc2ccccc2o1] |
| 206 | 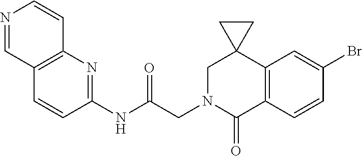 [SMILES: O=C(CN1CC2(CC2)c2cc(Br)ccc2C1=O)Nc1ccc2cnccc2n1] |
| 207 | 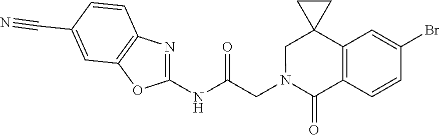 [SMILES: N#Cc1ccc2nc(NC(=O)CN3CC4(CC4)c4cc(Br)ccc4C3=O)oc2c1] |
| 208 | 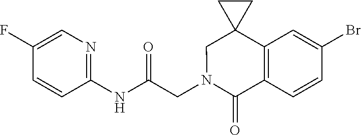 [SMILES: O=C(CN1CC2(CC2)c2cc(Br)ccc2C1=O)Nc1ccc(F)cn1] |
| 209 | 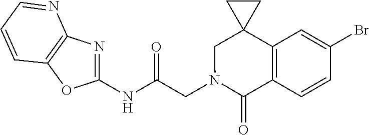 [SMILES: O=C(CN1CC2(CC2)c2cc(Br)ccc2C1=O)Nc1nc2ncccc2o1] |
| 210 | 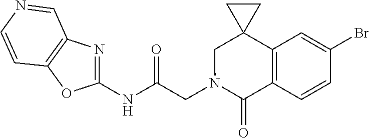 [SMILES: O=C(CN1CC2(CC2)c2cc(Br)ccc2C1=O)Nc1nc2cnccc2o1] |
| 211 | 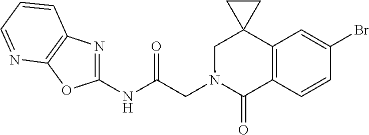 [SMILES: O=C(CN1CC2(CC2)c2cc(Br)ccc2C1=O)Nc1nc2cccnc2o1] |
| 212 | 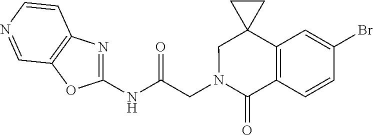 [SMILES: O=C(CN1CC2(CC2)c2cc(Br)ccc2C1=O)Nc1nc2ccncc2o1] |
| 213 | 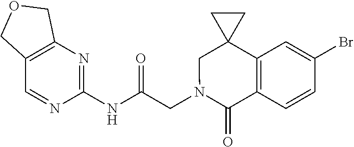 [SMILES: O=C(CN1CC2(CC2)c2cc(Br)ccc2C1=O)Nc1ncc2c(n1)COC2] |
| 214 | 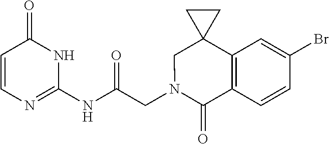 [SMILES: O=C(CN1CC2(CC2)c2cc(Br)ccc2C1=O)Nc1nccc(=O)[nH]1] |
| 215 | 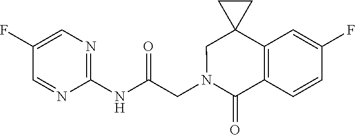 [SMILES: O=C(CN1CC2(CC2)c2cc(F)ccc2C1=O)Nc1ncc(F)cn1] |
| 216 | 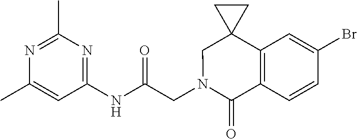 [SMILES: Cc1cc(NC(=O)CN2CC3(CC3)c3cc(Br)ccc3C2=O)nc(C)n1] |
| 217 | 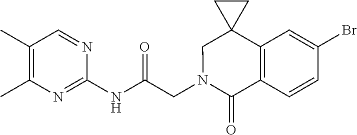 [SMILES: Cc1cnc(NC(=O)CN2CC3(CC3)c3cc(Br)ccc3C2=O)nc1C] |
| 218 | 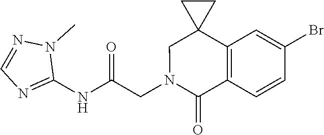 [SMILES: Cn1ncnc1NC(=O)CN1CC2(CC2)c2cc(Br)ccc2C1=O] |
| 219 | 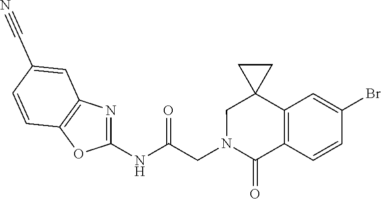 [SMILES: N#Cc1ccc2oc(NC(=O)CN3CC4(CC4)c4cc(Br)ccc4C3=O)nc2c1] |
| 220 | 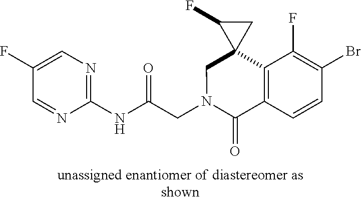 |
| 221 | 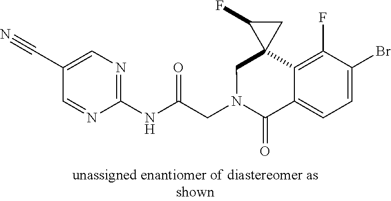 |
| 222 | 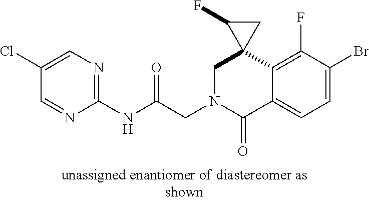 |
| 223 | 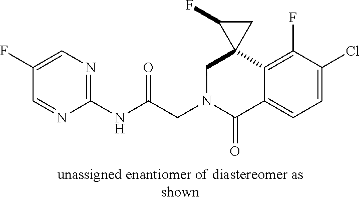 |
| 224 | 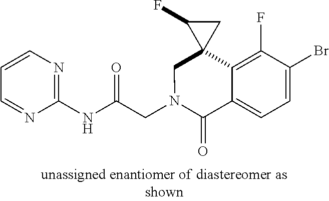 |
| 225 | 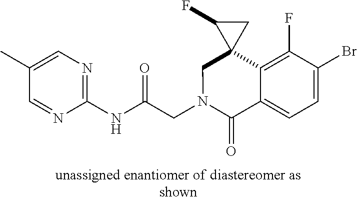 |
| 226 | 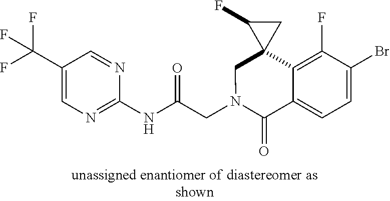 |
| 227 | 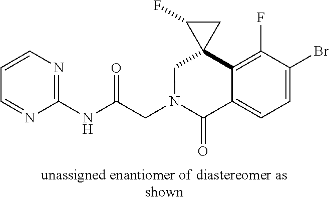 |
| 228 | 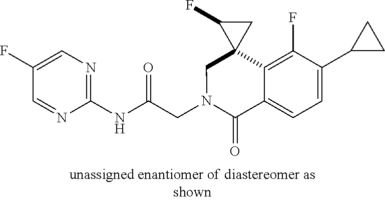 |
| 229 | 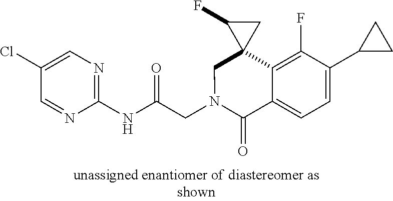 |
| 230 | 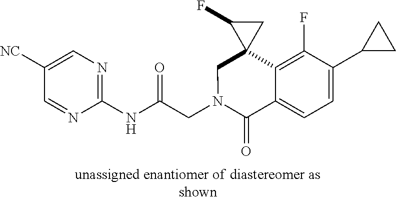 |
| 231 | 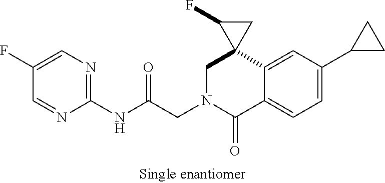 |
| 232 | 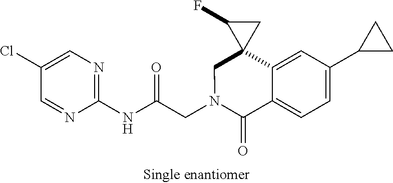 |
| 233 | 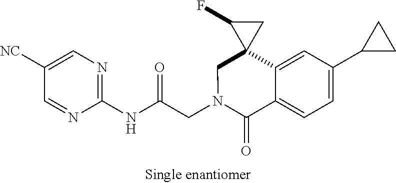 |
| 234 | 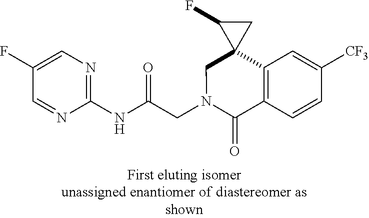 |
| 235 | 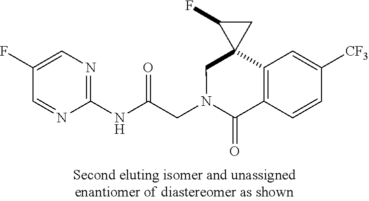 |
| 236 | 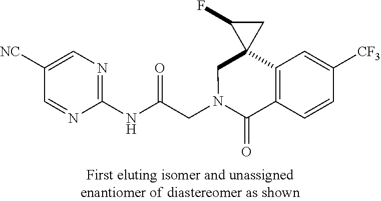 |
| 237 | 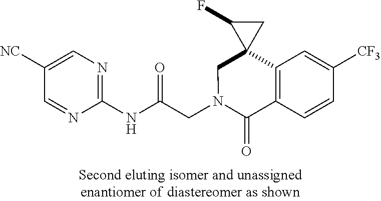 |
| 238 | 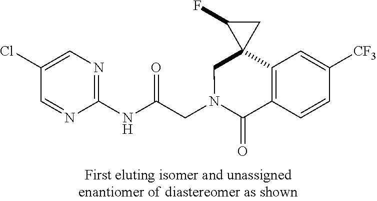 |
| 239 | 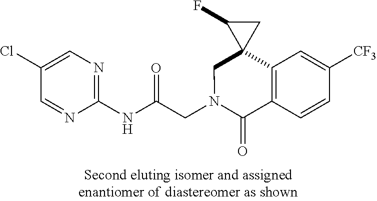 |
| 240 | 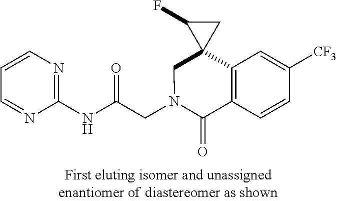 |
| 241 | 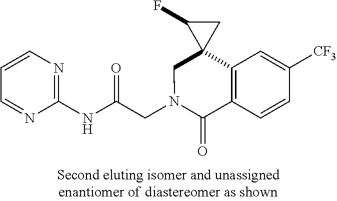 |
| 242 | 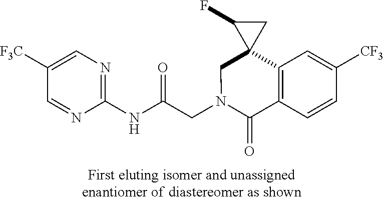 |
| 243 | 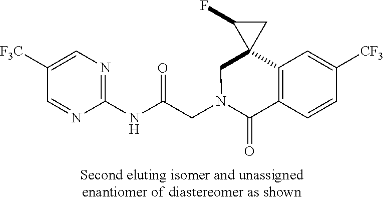 |
| 244 | 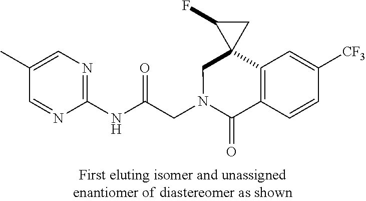 |
| 245 | 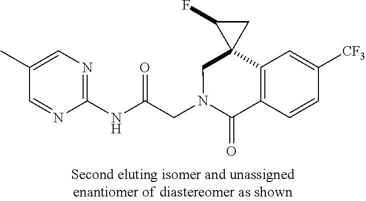 |
| 246 | 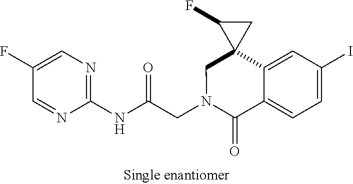 |
| 247 | 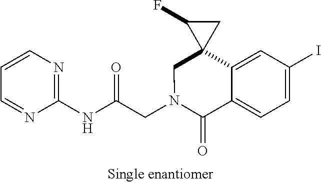 |
| 248 | 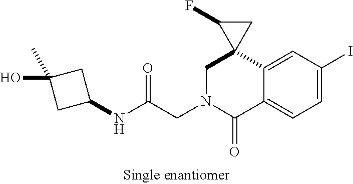 |
| 249 | 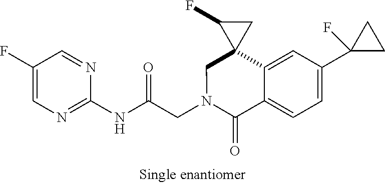 |
| 250 | 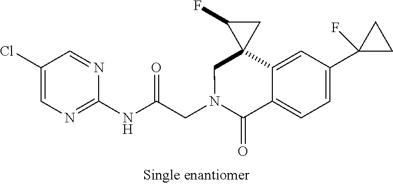 |
| 251 | 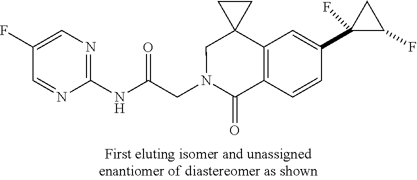 |
| 252 | 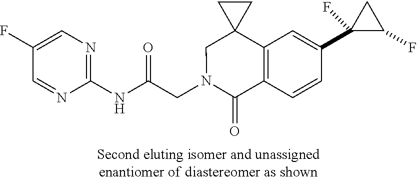 |
| 253 | 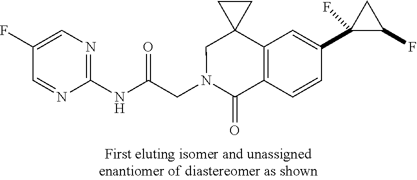 |
| 254 | 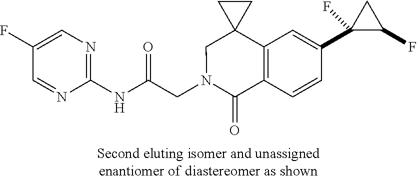 |
| 255 | 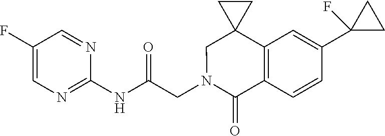 [SMILES: O=C(CN1CC2(CC2)c2cc(C3(F)CC3)ccc2C1=O)Nc1ncc(F)cn1] |
| 256 | 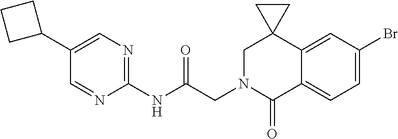 [SMILES: O=C(CN1CC2(CC2)c2cc(Br)ccc2C1=O)Nc1ncc(C2CCC2)cn1] |
| 257 | 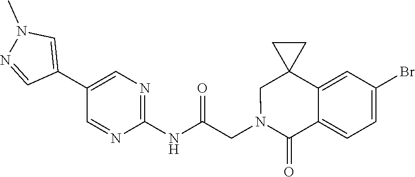 [SMILES: Cn1cc(-c2cnc(NC(=O)CN3CC4(CC4)c4cc(Br)ccc4C3=O)nc2)cn1] |
| 258 | 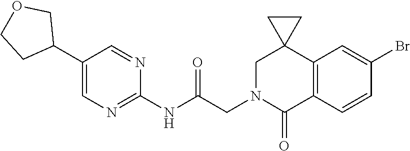 [SMILES: O=C(CN1CC2(CC2)c2cc(Br)ccc2C1=O)Nc1ncc(C2CCOC2)cn1] |
| 259 | 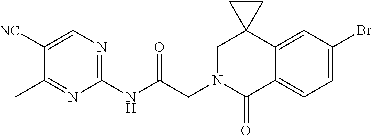 [SMILES: Cc1nc(NC(=O)CN2CC3(CC3)c3cc(Br)ccc3C2=O)ncc1C#N] |
| 260 | 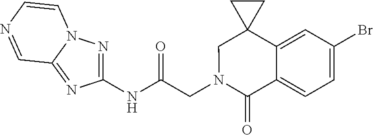 [SMILES: O=C(CN1CC2(CC2)c2cc(Br)ccc2C1=O)Nc1nc2cnccn2n1] |
| 261 | 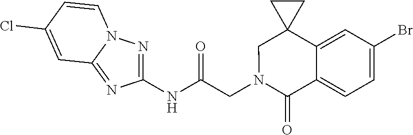 [SMILES: O=C(CN1CC2(CC2)c2cc(Br)ccc2C1=O)Nc1nc2cc(Cl)ccn2n1] |
| 262 | 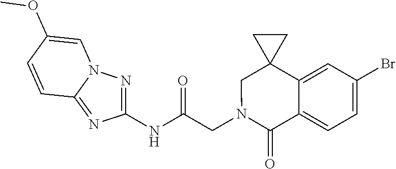 [SMILES: COc1ccc2nc(NC(=O)CN3CC4(CC4)c4cc(Br)ccc4C3=O)nn2c1] |
| 263 | 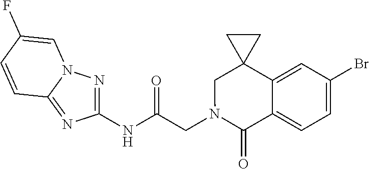 [SMILES: O=C(CN1CC2(CC2)c2cc(Br)ccc2C1=O)Nc1nc2ccc(F)cn2n1] |
| 264 | 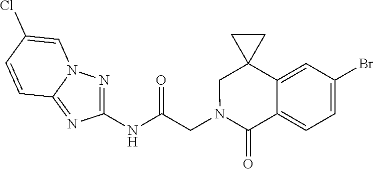 [SMILES: O=C(CN1CC2(CC2)c2cc(Br)ccc2C1=O)Nc1nc2ccc(Cl)cn2n1] |
| 265 | 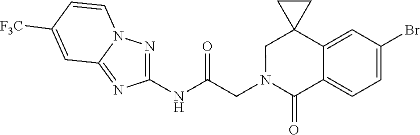 [SMILES: O=C(CN1CC2(CC2)c2cc(Br)ccc2C1=O)Nc1nc2cc(C(F)(F)F)ccn2n1] |
| 267 | 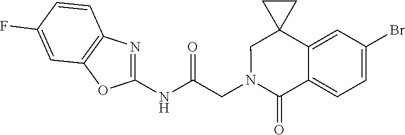 [SMILES: O=C(CN1CC2(CC2)c2cc(Br)ccc2C1=O)Nc1nc2ccc(F)cc2o1] |
| 268 | 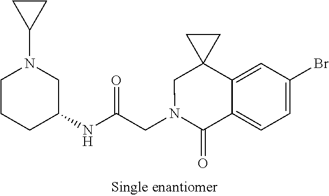 [SMILES: O=C(CN1CC2(CC2)c2cc(Br)ccc2C1=O)N[C@@H]1CCCN(C2CC2)C1] |
| 269 | 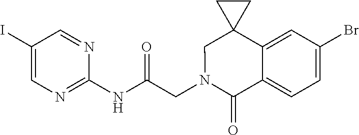 [SMILES: O=C(CN1CC2(CC2)c2cc(Br)ccc2C1=O)Nc1ncc(I)cn1] |
| 270 | 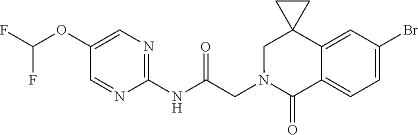 [SMILES: O=C(CN1CC2(CC2)c2cc(Br)ccc2C1=O)Nc1ncc(OC(F)F)cn1] |
| 271 | 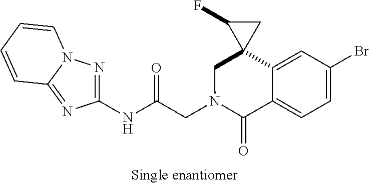 |
| 272 | 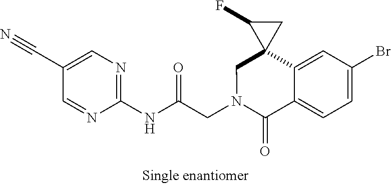 |
| 273 | 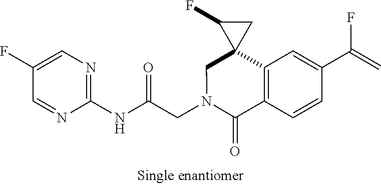 |
| 274 | 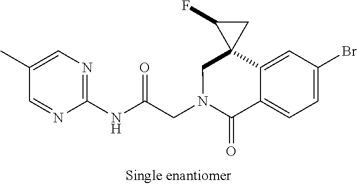 |
| 275 | 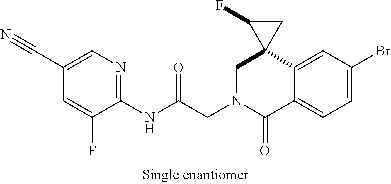 |
| 276 | 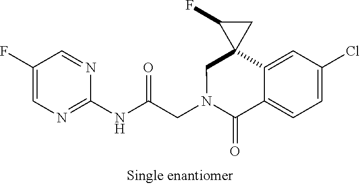 |
| 277 | 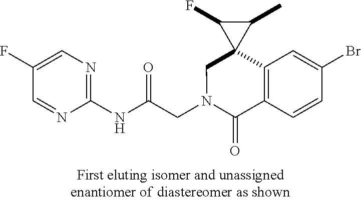 |
| 278 | 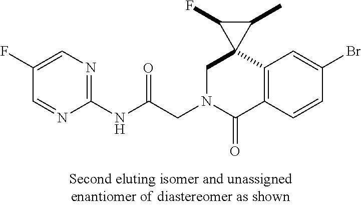 [SMILES: C[C@@H]1[C@H](F)[C@]12CN(CC(=O)Nc1ncc(F)cn1)C(=O)c1ccc(Br)cc12] |
| 279 | 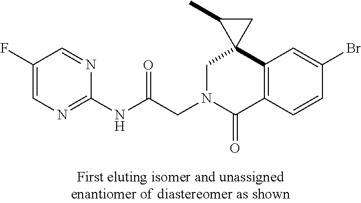 [SMILES: C[C@H]1C[C@]12CN(CC(=O)Nc1ncc(F)cn1)C(=O)c1ccc(Br)cc12] |
| 280 | 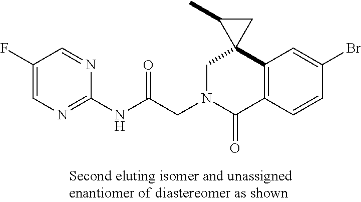 [SMILES: C[C@H]1C[C@]12CN(CC(=O)Nc1ncc(F)cn1)C(=O)c1ccc(Br)cc12] |
| 281 | 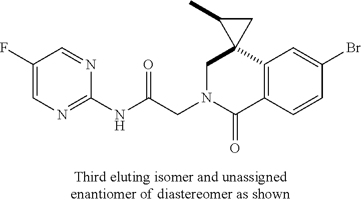 [SMILES: C[C@H]1C[C@@]12CN(CC(=O)Nc1ncc(F)cn1)C(=O)c1ccc(Br)cc12] |
| 282 | 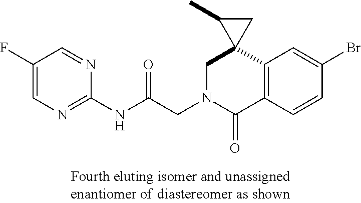 [SMILES: C[C@H]1C[C@@]12CN(CC(=O)Nc1ncc(F)cn1)C(=O)c1ccc(Br)cc12] |
| 283 | 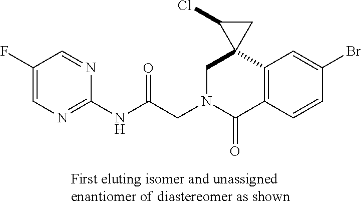 [SMILES: O=C(CN1C[C@@]2(C[C@@H]2Cl)c2cc(Br)ccc2C1=O)Nc1ncc(F)cn1] |
| 284 | 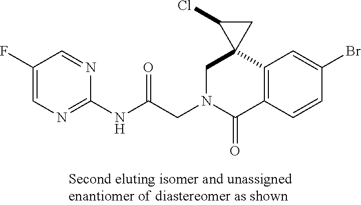 [SMILES: O=C(CN1C[C@@]2(C[C@@H]2Cl)c2cc(Br)ccc2C1=O)Nc1ncc(F)cn1] |
| 285 | 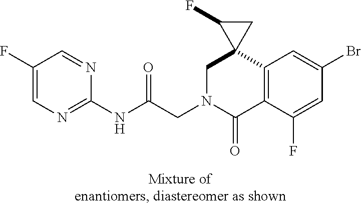 [SMILES: O=C(CN1C[C@@]2(C[C@@H]2F)c2cc(Br)cc(F)c2C1=O)Nc1ncc(F)cn1] |
| 286 | 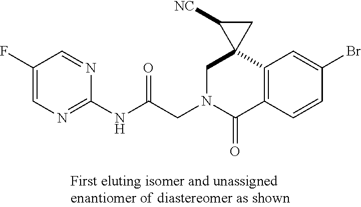 [SMILES: N#C[C@H]1C[C@@]12CN(CC(=O)Nc1ncc(F)cn1)C(=O)c1ccc(Br)cc12] |
| 287 | 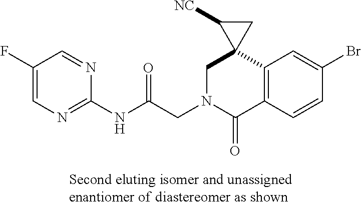 [SMILES: N#C[C@H]1C[C@@]12CN(CC(=O)Nc1ncc(F)cn1)C(=O)c1ccc(Br)cc12] |
| 288 | 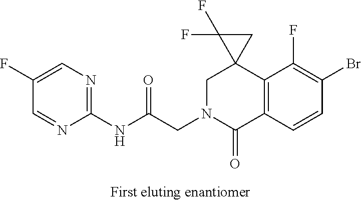 [SMILES: O=C(CN1CC2(CC2(F)F)c2c(ccc(Br)c2F)C1=O)Nc1ncc(F)cn1] |
| 289 | 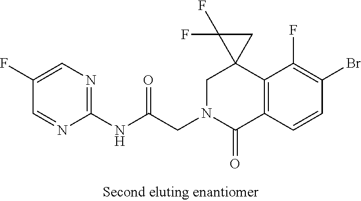 [SMILES: O=C(CN1CC2(CC2(F)F)c2c(ccc(Br)c2F)C1=O)Nc1ncc(F)cn1] |
| 290 | 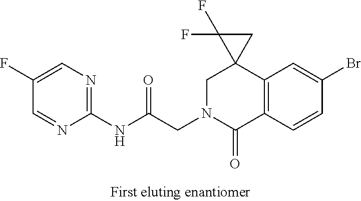 [SMILES: O=C(CN1CC2(CC2(F)F)c2cc(Br)ccc2C1=O)Nc1ncc(F)cn1] |
| 291 | 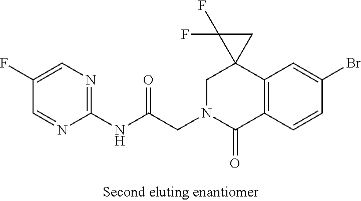 [SMILES: O=C(CN1CC2(CC2(F)F)c2cc(Br)ccc2C1=O)Nc1ncc(F)cn1] |
| 292 | 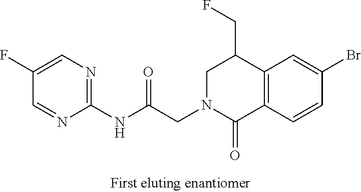 [SMILES: O=C(CN1CC(CF)c2cc(Br)ccc2C1=O)Nc1ncc(F)cn1] |
| 293 | 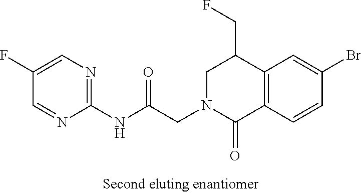 [SMILES: O=C(CN1CC(CF)c2cc(Br)ccc2C1=O)Nc1ncc(F)cn1] |
| 294 | 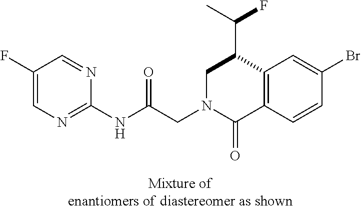 |
| 295 | 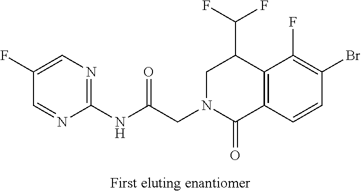 [SMILES: O=C(CN1CC(C(F)F)c2c(ccc(Br)c2F)C1=O)Nc1ncc(F)cn1] |
| 296 | 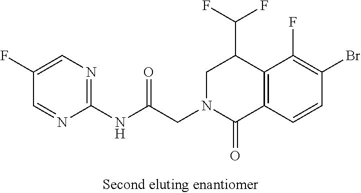 [SMILES: O=C(CN1CC(C(F)F)c2c(ccc(Br)c2F)C1=O)Nc1ncc(F)cn1] |
| 297 | 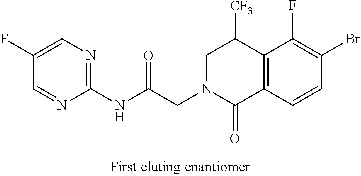 [SMILES: O=C(CN1CC(C(F)(F)F)c2c(ccc(Br)c2F)C1=O)Nc1ncc(F)cn1] |
| 298 | 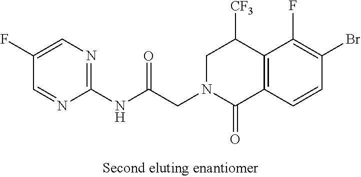 [SMILES: O=C(CN1CC(C(F)(F)F)c2c(ccc(Br)c2F)C1=O)Nc1ncc(F)cn1] |
| 299 | 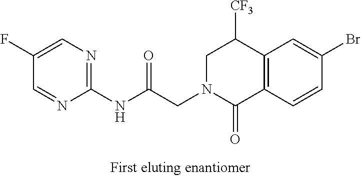 [SMILES: O=C(CN1CC(C(F)(F)F)c2cc(Br)ccc2C1=O)Nc1ncc(F)cn1] |
| 300 | 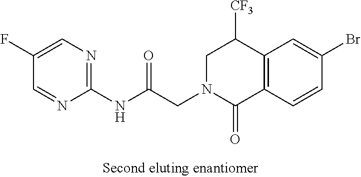 [SMILES: O=C(CN1CC(C(F)(F)F)c2cc(Br)ccc2C1=O)Nc1ncc(F)cn1] |
| 301 | 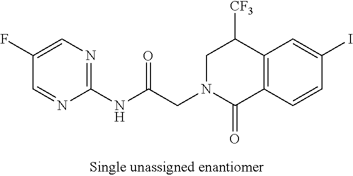 [SMILES: O=C(CN1CC(C(F)(F)F)c2cc(I)ccc2C1=O)Nc1ncc(F)cn1] |
| 302 | 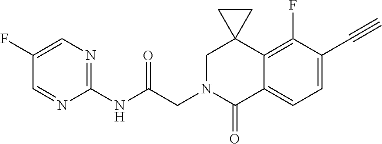 [SMILES: C#Cc1ccc2c(c1F)C1(CC1)CN(CC(=O)Nc1ncc(F)cn1)C2=O] |
| 303 | 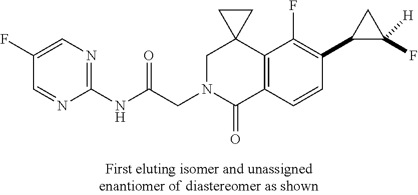 |
| 304 | 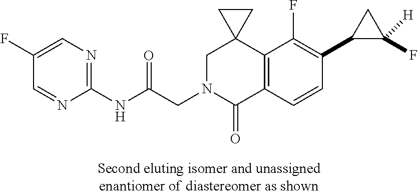 |
| 305 | 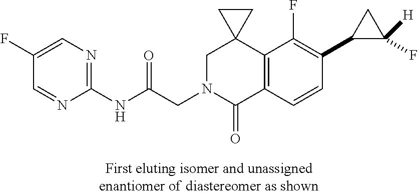 |
| 306 | 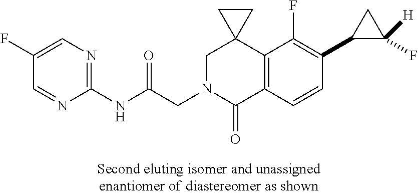 |
| 307 | 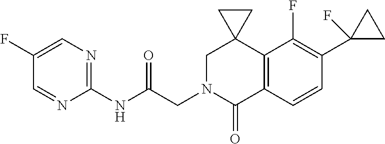 [SMILES: O=C(CN1CC2(CC2)c2c(ccc(C3(F)CC3)c2F)C1=O)Nc1ncc(F)cn1] |
| 308 | 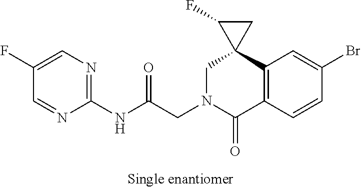 [SMILES: O=C(CN1C[C@]2(C[C@H]2F)c2cc(Br)ccc2C1=O)Nc1ncc(F)cn1] |
| 309 | 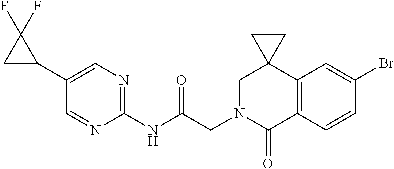 [SMILES: O=C(CN1CC2(CC2)c2cc(Br)ccc2C1=O)Nc1ncc(C2CC2(F)F)cn1] |
| 310 | 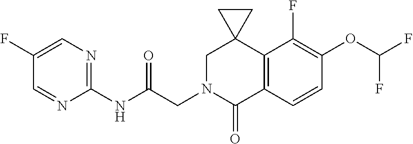 [SMILES: O=C(CN1CC2(CC2)c2c(ccc(OC(F)F)c2F)C1=O)Nc1ncc(F)cn1] |
| 311 | 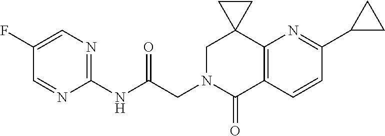 [SMILES: O=C(CN1CC2(CC2)c2nc(C3CC3)ccc2C1=O)Nc1ncc(F)cn1] |
| 312 | 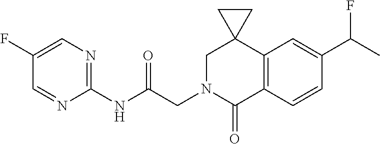 [SMILES: CC(F)c1ccc2c(c1)C1(CC1)CN(CC(=O)Nc1ncc(F)cn1)C2=O] |
| 313 | 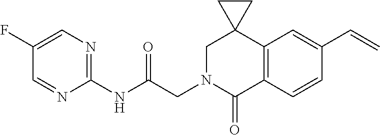 [SMILES: C=Cc1ccc2c(c1)C1(CC1)CN(CC(=O)Nc1ncc(F)cn1)C2=O] |
| 314 | 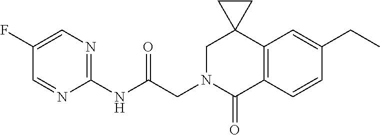 [SMILES: CCc1ccc2c(c1)C1(CC1)CN(CC(=O)Nc1ncc(F)cn1)C2=O] |
| 315 | 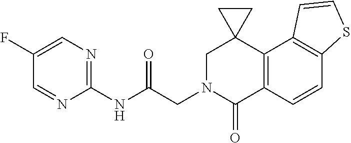 [SMILES: O=C(CN1CC2(CC2)c2c(ccc3sccc23)C1=O)Nc1ncc(F)cn1] |
| 316 | 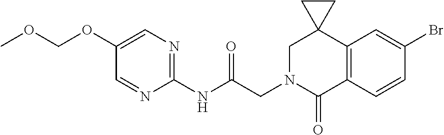 [SMILES: COCOc1cnc(NC(=O)CN2CC3(CC3)c3cc(Br)ccc3C2=O)nc1] |
| 317 | 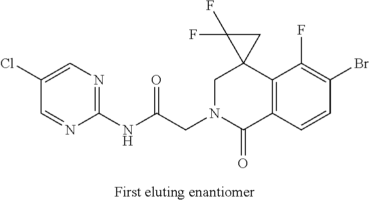 [SMILES: O=C(CN1CC2(CC2(F)F)c2c(ccc(Br)c2F)C1=O)Nc1ncc(Cl)cn1] |
| 318 | 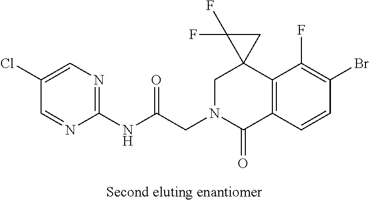 [SMILES: O=C(CN1CC2(CC2(F)F)c2c(ccc(Br)c2F)C1=O)Nc1ncc(Cl)cn1] |
| 319 | 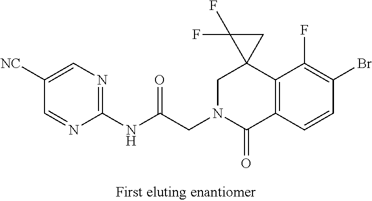 [SMILES: N#Cc1cnc(NC(=O)CN2CC3(CC3(F)F)c3c(ccc(Br)c3F)C2=O)nc1] |
| 320 | 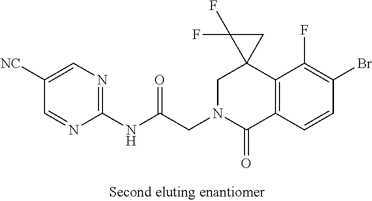 [SMILES: N#Cc1cnc(NC(=O)CN2CC3(CC3(F)F)c3c(ccc(Br)c3F)C2=O)nc1] |
| 321 | 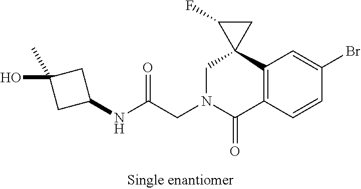 |
| 322 | 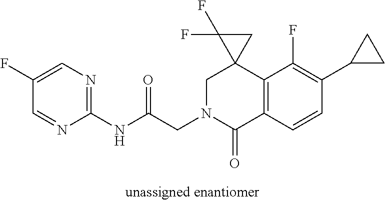 [SMILES: O=C(CN1CC2(CC2(F)F)c2c(ccc(C3CC3)c2F)C1=O)Nc1ncc(F)cn1] |
| 323 | 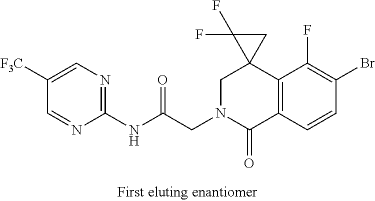 [SMILES: O=C(CN1CC2(CC2(F)F)c2c(ccc(Br)c2F)C1=O)Nc1ncc(C(F)(F)F)cn1] |
| 324 | 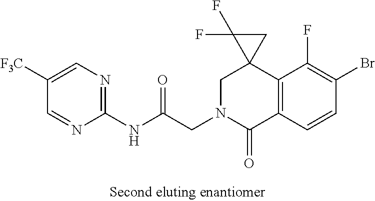 [SMILES: O=C(CN1CC2(CC2(F)F)c2c(ccc(Br)c2F)C1=O)Nc1ncc(C(F)(F)F)cn1] |
| 325 | 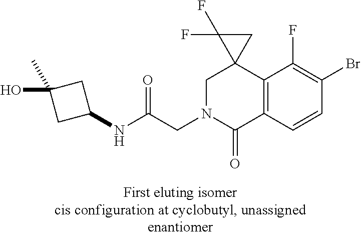 |
| 326 | 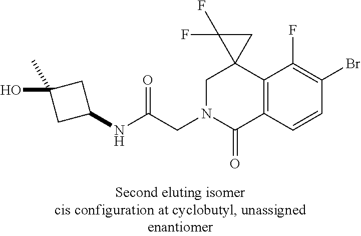 |
| 327 | 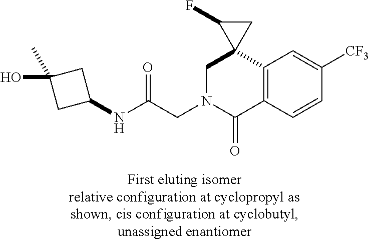 |
| 328 | 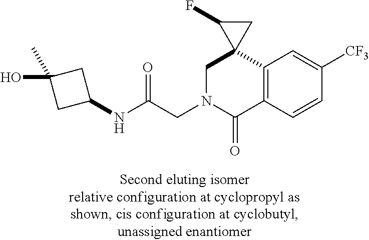 |
| 329 | 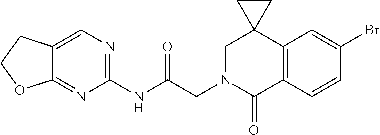 [SMILES: O=C(CN1CC2(CC2)c2cc(Br)ccc2C1=O)Nc1ncc2c(n1)OCC2] |
| 330 | 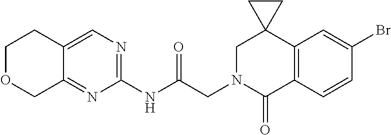 [SMILES: O=C(CN1CC2(CC2)c2cc(Br)ccc2C1=O)Nc1ncc2c(n1)COCC2] |
| 331 | 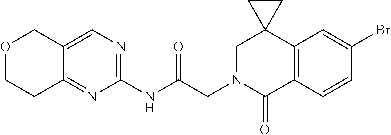 [SMILES: O=C(CN1CC2(CC2)c2cc(Br)ccc2C1=O)Nc1ncc2c(n1)CCOC2] |
| 332 | 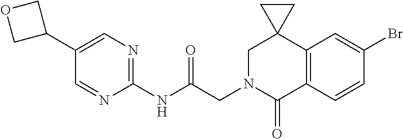 [SMILES: O=C(CN1CC2(CC2)c2cc(Br)ccc2C1=O)Nc1ncc(C2COC2)cn1] |
| 333 | 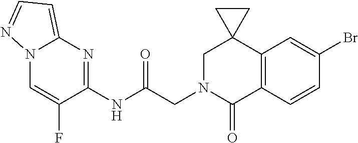 [SMILES: O=C(CN1CC2(CC2)c2cc(Br)ccc2C1=O)Nc1nc2ccnn2cc1F] |
| 334 | 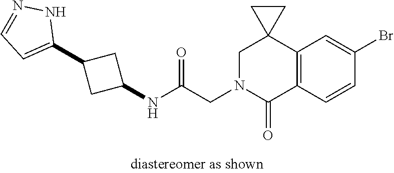 [SMILES: O=C(CN1CC2(CC2)c2cc(Br)ccc2C1=O)N[C@H]1C[C@@H](c2ccn[nH]2)C1] |
| 335 | 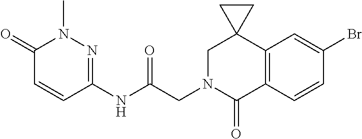 [SMILES: Cn1nc(NC(=O)CN2CC3(CC3)c3cc(Br)ccc3C2=O)ccc1=O] |
| 336 | 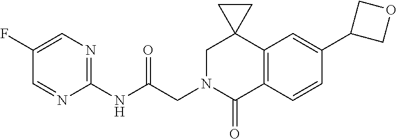 [SMILES: O=C(CN1CC2(CC2)c2cc(C3COC3)ccc2C1=O)Nc1ncc(F)cn1] |
| 337 | 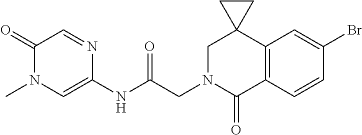 [SMILES: Cn1cc(NC(=O)CN2CC3(CC3)c3cc(Br)ccc3C2=O)ncc1=O] |
| 338 | 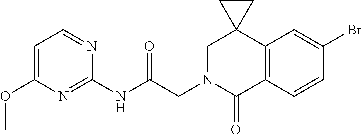 [SMILES: COc1ccnc(NC(=O)CN2CC3(CC3)c3cc(Br)ccc3C2=O)n1] |
| 339 | 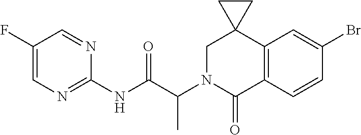 [SMILES: CC(C(=O)Nc1ncc(F)cn1)N1CC2(CC2)c2cc(Br)ccc2C1=O] |
| 340 | 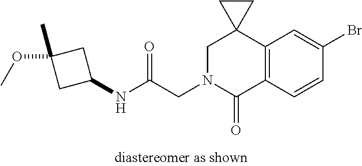 |
| 341 | 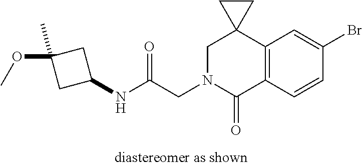 |
| 342 | 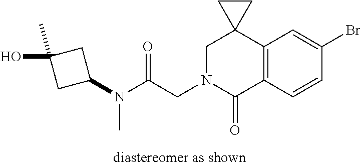 |
| 343 | 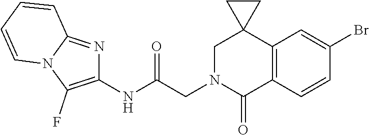 [SMILES: O=C(CN1CC2(CC2)c2cc(Br)ccc2C1=O)Nc1nc2ccccn2c1F] |
| 344 | 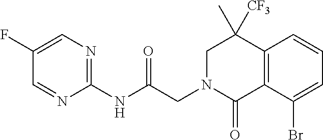 [SMILES: CC1(C(F)(F)F)CN(CC(=O)Nc2ncc(F)cn2)C(=O)c2c(Br)cccc21] |
| 345 | 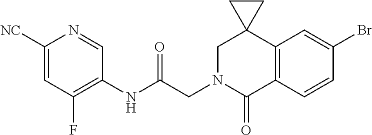 [SMILES: N#Cc1cc(F)c(NC(=O)CN2CC3(CC3)c3cc(Br)ccc3C2=O)cn1] |
| 346 | 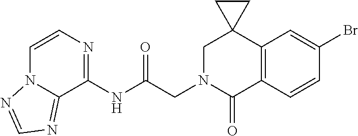 [SMILES: O=C(CN1CC2(CC2)c2cc(Br)ccc2C1=O)Nc1nccn2ncnc12] |
| 347 | 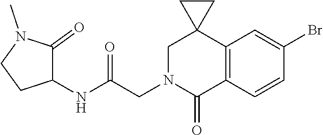 [SMILES: CN1CCC(NC(=O)CN2CC3(CC3)c3cc(Br)ccc3C2=O)C1=O] |
| 348 | 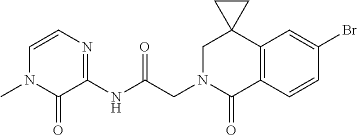 [SMILES: Cn1ccnc(NC(=O)CN2CC3(CC3)c3cc(Br)ccc3C2=O)c1=O] |
| 349 | 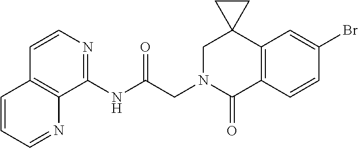 [SMILES: O=C(CN1CC2(CC2)c2cc(Br)ccc2C1=O)Nc1nccc2cccnc12] |
| 350 | 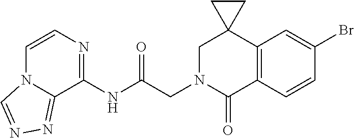 [SMILES: O=C(CN1CC2(CC2)c2cc(Br)ccc2C1=O)Nc1nccn2cnnc12] |
| 351 | 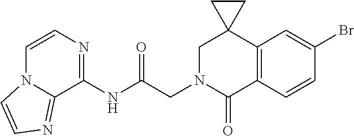 [SMILES: O=C(CN1CC2(CC2)c2cc(Br)ccc2C1=O)Nc1nccn2ccnc12] |
| 352 | 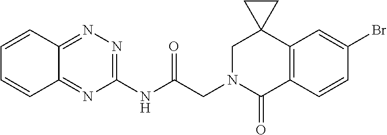 [SMILES: O=C(CN1CC2(CC2)c2cc(Br)ccc2C1=O)Nc1nnc2ccccc2n1] |
| 353 | 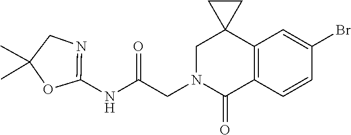 [SMILES: CC1(C)CN=C(NC(=O)CN2CC3(CC3)c3cc(Br)ccc3C2=O)O1] |
| 354 | 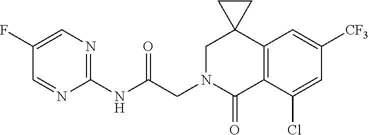 [SMILES: O=C(CN1CC2(CC2)c2cc(C(F)(F)F)cc(Cl)c2C1=O)Nc1ncc(F)cn1] |
| 355 | 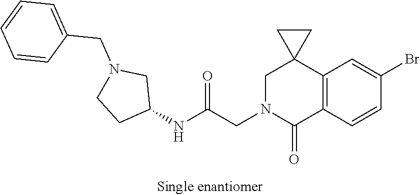 [SMILES: O=C(CN1CC2(CC2)c2cc(Br)ccc2C1=O)N[C@@H]1CCN(Cc2ccccc2)C1] |
| 356 | 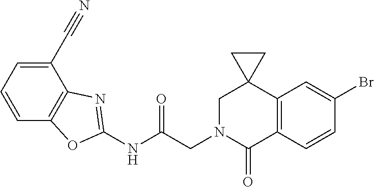 [SMILES: N#Cc1cccc2oc(NC(=O)CN3CC4(CC4)c4cc(Br)ccc4C3=O)nc12] |
| 357 | 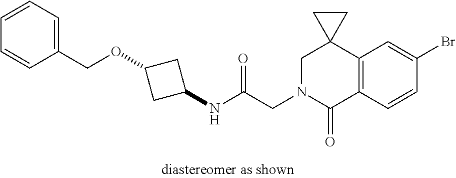 |
| 358 | 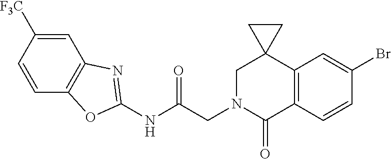 [SMILES: O=C(CN1CC2(CC2)c2cc(Br)ccc2C1=O)Nc1nc2cc(C(F)(F)F)ccc2o1] |
| 359 | 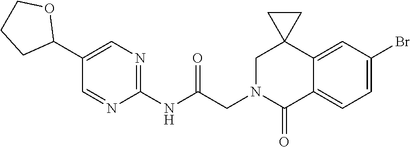 [SMILES: O=C(CN1CC2(CC2)c2cc(Br)ccc2C1=O)Nc1ncc(C2CCCO2)cn1] |
| 360 | 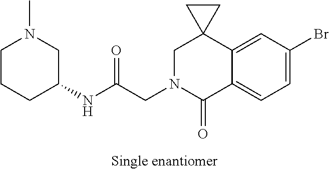 [SMILES: CN1CCC[C@@H](NC(=O)CN2CC3(CC3)c3cc(Br)ccc3C2=O)C1] |
| 361 | 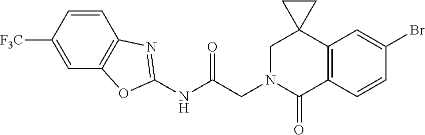 [SMILES: O=C(CN1CC2(CC2)c2cc(Br)ccc2C1=O)Nc1nc2ccc(C(F)(F)F)cc2o1] |
| 362 | 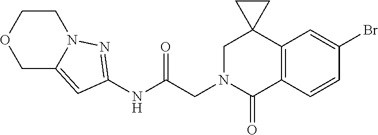 [SMILES: O=C(CN1CC2(CC2)c2cc(Br)ccc2C1=O)Nc1cc2n(n1)CCOC2] |
| 363 | 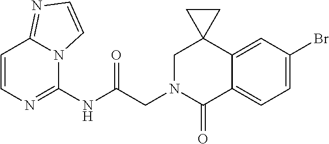 [SMILES: O=C(CN1CC2(CC2)c2cc(Br)ccc2C1=O)Nc1nccc2nccn12] |
| 364 | 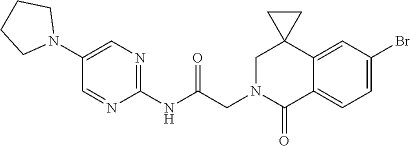 [SMILES: O=C(CN1CC2(CC2)c2cc(Br)ccc2C1=O)Nc1ncc(N2CCCC2)cn1] |
| 365 | 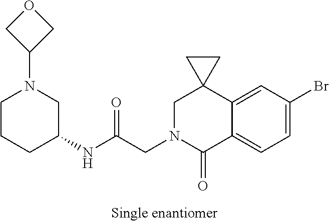 [SMILES: O=C(CN1CC2(CC2)c2cc(Br)ccc2C1=O)N[C@@H]1CCCN(C2COC2)C1] |
| 366 | 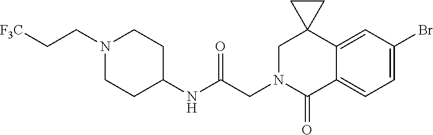 [SMILES: O=C(CN1CC2(CC2)c2cc(Br)ccc2C1=O)NC1CCN(CCC(F)(F)F)CC1] |
| 367 | 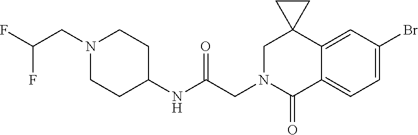 [SMILES: O=C(CN1CC2(CC2)c2cc(Br)ccc2C1=O)NC1CCN(CC(F)F)CC1] |
| 368 | 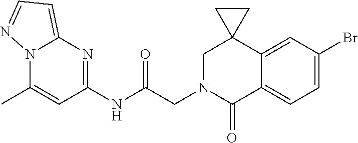 [SMILES: Cc1cc(NC(=O)CN2CC3(CC3)c3cc(Br)ccc3C2=O)nc2ccnn12] |
| 369 | 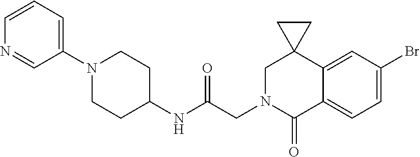 [SMILES: O=C(CN1CC2(CC2)c2cc(Br)ccc2C1=O)NC1CCN(c2cccnc2)CC1] |
| 370 | 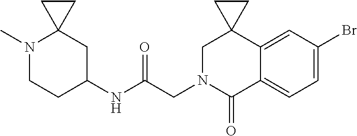 [SMILES: CN1CCC(NC(=O)CN2CC3(CC3)c3cc(Br)ccc3C2=O)CC12CC2] |
| 371 | 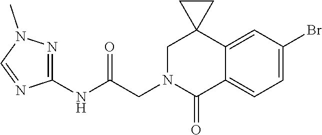 [SMILES: Cn1cnc(NC(=O)CN2CC3(CC3)c3cc(Br)ccc3C2=O)n1] |
| 372 | 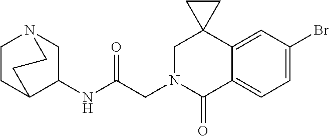 [SMILES: O=C(CN1CC2(CC2)c2cc(Br)ccc2C1=O)NC1CN2CCC1CC2] |
| 373 | 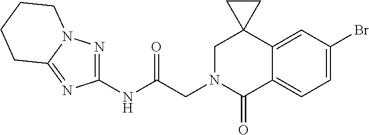 [SMILES: O=C(CN1CC2(CC2)c2cc(Br)ccc2C1=O)Nc1nc2n(n1)CCCC2] |
| 374 | 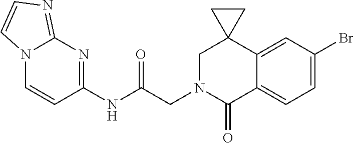 [SMILES: O=C(CN1CC2(CC2)c2cc(Br)ccc2C1=O)Nc1ccn2ccnc2n1] |
| 375 | 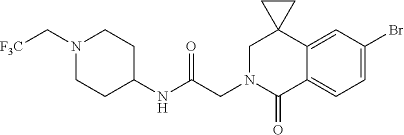 [SMILES: O=C(CN1CC2(CC2)c2cc(Br)ccc2C1=O)NC1CCN(CC(F)(F)F)CC1] |
| 376 | 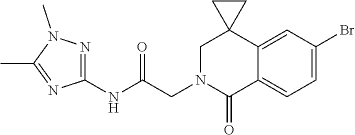 [SMILES: Cc1nc(NC(=O)CN2CC3(CC3)c3cc(Br)ccc3C2=O)nn1C] |
| 377 | 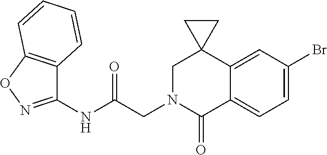 [SMILES: O=C(CN1CC2(CC2)c2cc(Br)ccc2C1=O)Nc1noc2ccccc12] |
| 378 | 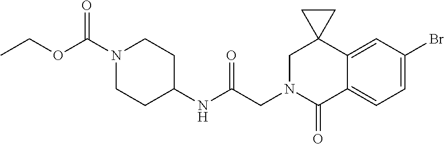 [SMILES: CCOC(=O)N1CCC(NC(=O)CN2CC3(CC3)c3cc(Br)ccc3C2=O)CC1] |
| 379 | 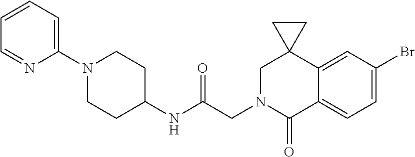 [SMILES: O=C(CN1CC2(CC2)c2cc(Br)ccc2C1=O)NC1CCN(c2ccccn2)CC1] |
| 380 | 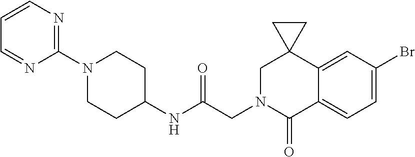 [SMILES: O=C(CN1CC2(CC2)c2cc(Br)ccc2C1=O)NC1CCN(c2ncccn2)CC1] |
| 381 | 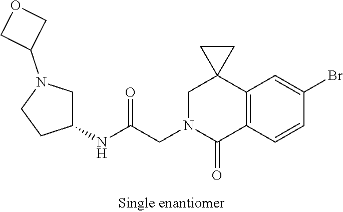 [SMILES: O=C(CN1CC2(CC2)c2cc(Br)ccc2C1=O)N[C@@H]1CCN(C2COC2)C1] |
| 382 | 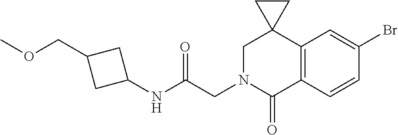 [SMILES: COCC1CC(NC(=O)CN2CC3(CC3)c3cc(Br)ccc3C2=O)C1] |
| 383 | 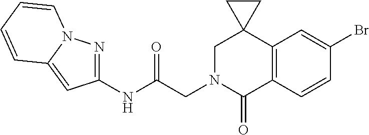 [SMILES: O=C(CN1CC2(CC2)c2cc(Br)ccc2C1=O)Nc1cc2ccccn2n1] |
| 384 | 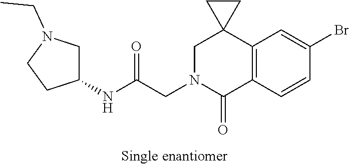 [SMILES: CCN1CC[C@@H](NC(=O)CN2CC3(CC3)c3cc(Br)ccc3C2=O)C1] |
| 385 | 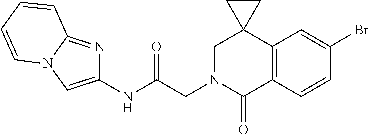 [SMILES: O=C(CN1CC2(CC2)c2cc(Br)ccc2C1=O)Nc1cn2ccccc2n1] |
| 386 | 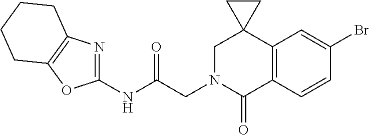 [SMILES: O=C(CN1CC2(CC2)c2cc(Br)ccc2C1=O)Nc1nc2c(o1)CCCC2] |
| 387 | 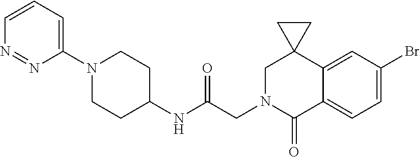 [SMILES: O=C(CN1CC2(CC2)c2cc(Br)ccc2C1=O)NC1CCN(c2cccnn2)CC1] |
| 389 | 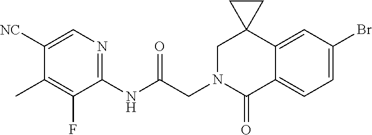 [SMILES: Cc1c(C#N)cnc(NC(=O)CN2CC3(CC3)c3cc(Br)ccc3C2=O)c1F] |
| 390 | 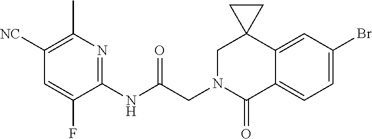 [SMILES: Cc1nc(NC(=O)CN2CC3(CC3)c3cc(Br)ccc3C2=O)c(F)cc1C#N] |
| 391 | 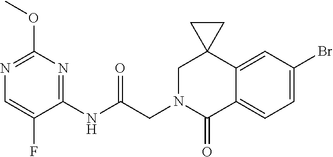 [SMILES: COc1ncc(F)c(NC(=O)CN2CC3(CC3)c3cc(Br)ccc3C2=O)n1] |
| 392 | 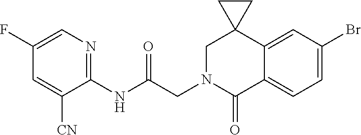 [SMILES: N#Cc1cc(F)cnc1NC(=O)CN1CC2(CC2)c2cc(Br)ccc2C1=O] |
| 393 | 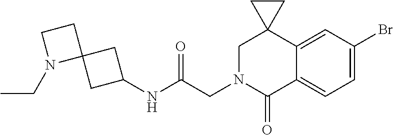 [SMILES: CCN1CCC12CC(NC(=O)CN1CC3(CC3)c3cc(Br)ccc3C1=O)C2] |
| 394 | 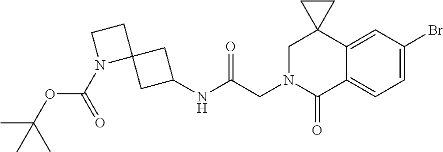 [SMILES: CC(C)(C)OC(=O)N1CCC12CC(NC(=O)CN1CC3(CC3)c3cc(Br)ccc3C1=O)C2] |
| 395 | 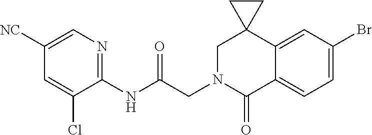 [SMILES: N#Cc1cnc(NC(=O)CN2CC3(CC3)c3cc(Br)ccc3C2=O)c(Cl)c1] |
| 396 | 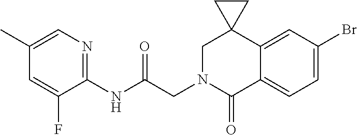 [SMILES: Cc1cnc(NC(=O)CN2CC3(CC3)c3cc(Br)ccc3C2=O)c(F)c1] |
| 397 | 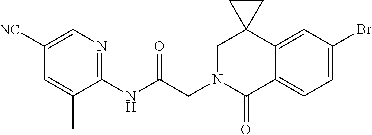 [SMILES: Cc1cc(C#N)cnc1NC(=O)CN1CC2(CC2)c2cc(Br)ccc2C1=O] |
| 398 | 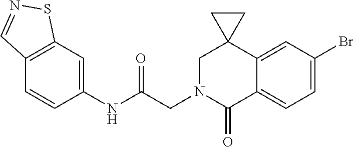 [SMILES: O=C(CN1CC2(CC2)c2cc(Br)ccc2C1=O)Nc1ccc2cnsc2c1] |
| 399 | 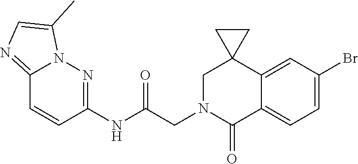 [SMILES: Cc1cnc2ccc(NC(=O)CN3CC4(CC4)c4cc(Br)ccc4C3=O)nn12] |
| 400 | 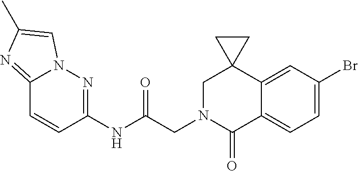 [SMILES: Cc1cn2nc(NC(=O)CN3CC4(CC4)c4cc(Br)ccc4C3=O)ccc2n1] |
| 401 | 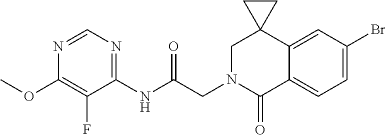 [SMILES: COc1ncnc(NC(=O)CN2CC3(CC3)c3cc(Br)ccc3C2=O)c1F] |
| 402 | 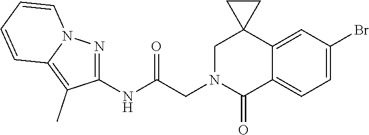 [SMILES: Cc1c(NC(=O)CN2CC3(CC3)c3cc(Br)ccc3C2=O)nn2ccccc12] |
| 403 | 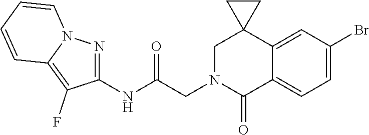 [SMILES: O=C(CN1CC2(CC2)c2cc(Br)ccc2C1=O)Nc1nn2ccccc2c1F] |
| 404 | 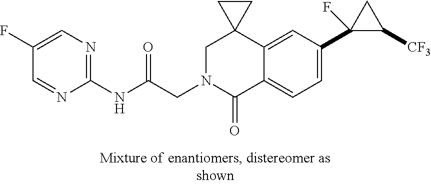 |
| 405 | 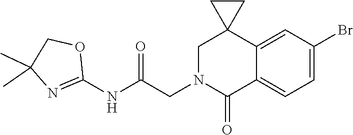 [SMILES: CC1(C)COC(NC(=O)CN2CC3(CC3)c3cc(Br)ccc3C2=O)=N1] |
| 406 | 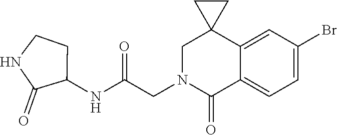 [SMILES: O=C(CN1CC2(CC2)c2cc(Br)ccc2C1=O)NC1CCNC1=O] |
| 407 | 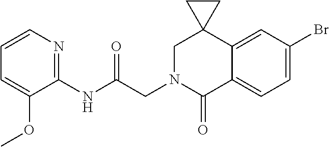 [SMILES: COc1cccnc1NC(=O)CN1CC2(CC2)c2cc(Br)ccc2C1=O] |
| 408 | 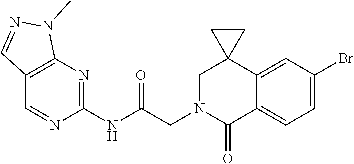 [SMILES: Cn1ncc2cnc(NC(=O)CN3CC4(CC4)c4cc(Br)ccc4C3=O)nc21] |
| 409 | 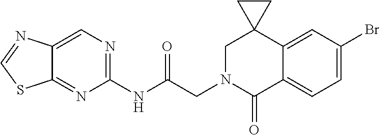 [SMILES: O=C(CN1CC2(CC2)c2cc(Br)ccc2C1=O)Nc1ncc2ncsc2n1] |
| 410 | 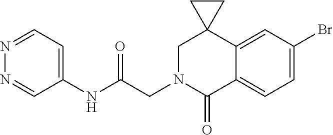 [SMILES: O=C(CN1CC2(CC2)c2cc(Br)ccc2C1=O)Nc1ccnnc1] |
| 411 | 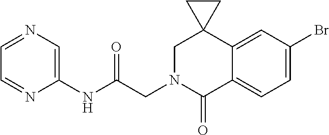 [SMILES: O=C(CN1CC2(CC2)c2cc(Br)ccc2C1=O)Nc1cnccn1] |
| 412 | 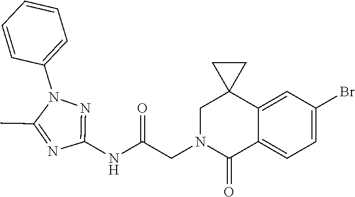 [SMILES: Cc1nc(NC(=O)CN2CC3(CC3)c3cc(Br)ccc3C2=O)nn1-c1ccccc1] |
| 413 | 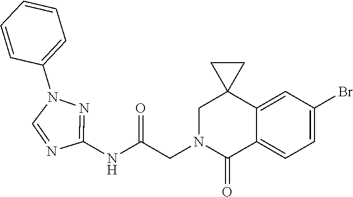 [SMILES: O=C(CN1CC2(CC2)c2cc(Br)ccc2C1=O)Nc1ncn(-c2ccccc2)n1] |
| 414 | 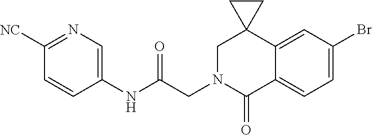 [SMILES: N#Cc1ccc(NC(=O)CN2CC3(CC3)c3cc(Br)ccc3C2=O)cn1] |
| 415 | 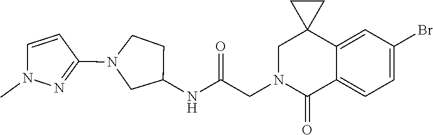 [SMILES: Cn1ccc(N2CCC(NC(=O)CN3CC4(CC4)c4cc(Br)ccc4C3=O)C2)n1] |
| 416 | 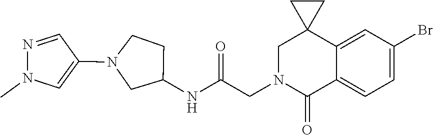 [SMILES: Cn1cc(N2CCC(NC(=O)CN3CC4(CC4)c4cc(Br)ccc4C3=O)C2)cn1] |
| 417 | 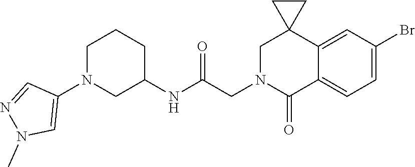 [SMILES: Cn1cc(N2CCCC(NC(=O)CN3CC4(CC4)c4cc(Br)ccc4C3=O)C2)cn1] |
| 418 | 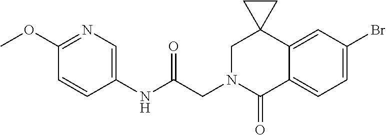 [SMILES: COc1ccc(NC(=O)CN2CC3(CC3)c3cc(Br)ccc3C2=O)cn1] |
| 419 | 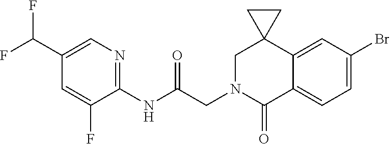 [SMILES: O=C(CN1CC2(CC2)c2cc(Br)ccc2C1=O)Nc1ncc(C(F)F)cc1F] |
| 420 | 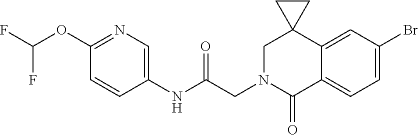 [SMILES: O=C(CN1CC2(CC2)c2cc(Br)ccc2C1=O)Nc1ccc(OC(F)F)nc1] |
| 421 | 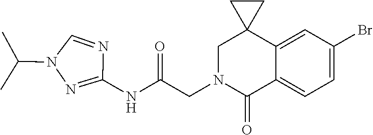 [SMILES: CC(C)n1cnc(NC(=O)CN2CC3(CC3)c3cc(Br)ccc3C2=O)n1] |
| 422 | 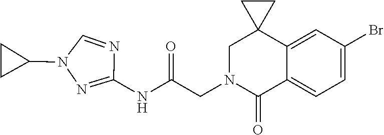 [SMILES: O=C(CN1CC2(CC2)c2cc(Br)ccc2C1=O)Nc1ncn(C2CC2)n1] |
| 423 | 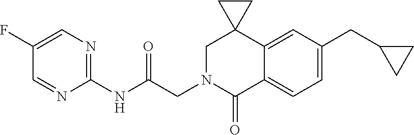 [SMILES: O=C(CN1CC2(CC2)c2cc(CC3CC3)ccc2C1=O)Nc1ncc(F)cn1] |
| 424 | 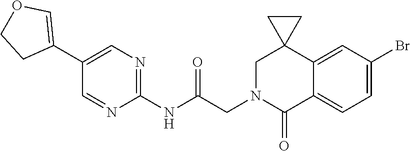 [SMILES: O=C(CN1CC2(CC2)c2cc(Br)ccc2C1=O)Nc1ncc(C2=COCC2)cn1] |
| 425 | 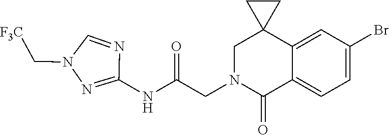 [SMILES: O=C(CN1CC2(CC2)c2cc(Br)ccc2C1=O)Nc1ncn(CC(F)(F)F)n1] |
| 427 | 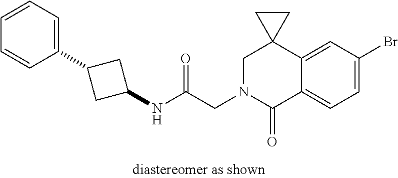 |
| 429 | 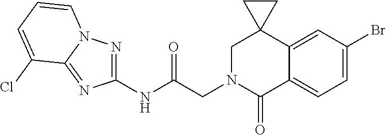 [SMILES: O=C(CN1CC2(CC2)c2cc(Br)ccc2C1=O)Nc1nc2c(Cl)cccn2n1] |
| 430 | 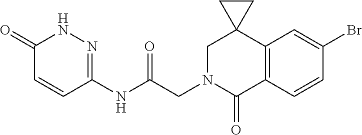 [SMILES: O=C(CN1CC2(CC2)c2cc(Br)ccc2C1=O)Nc1ccc(=O)[nH]n1] |

Absolute stereochemistry was assigned for certain compounds described herein as shown in Table 1A. Absolute stereochemistry was determined by co-crystallization using methods adapted from Sharif H., et al. Structural mechanism for NEK7-licensed activation of NLRP3 inflammasome. Nature, 2019, 570(7761), 338-343, or by correlation to an assigned compound via the use of specific enantiomerically enriched starting materials. Accordingly, in certain embodiments, provided is a compound of Table 1A, or a pharmaceutically acceptable salt, isotopically enriched analog, or prodrug thereof:

TABLE 1A

| Ex. | Structure | Ex. | Structure |
|---|---|---|---|
| 78 | 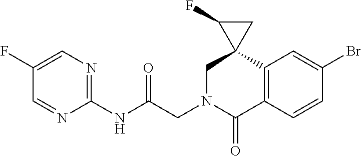 [SMILES: O=C(CN1C[C@@]2(C[C@@H]2F)c2cc(Br)ccc2C1=O)Nc1ncc(F)cn1] | 231 | 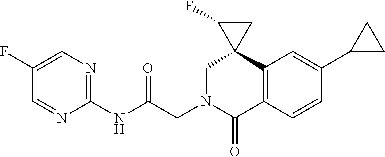 [SMILES: O=C(CN1C[C@]2(C[C@H]2F)c2cc(C3CC3)ccc2C1=O)Nc1ncc(F)cn1] |
| 79 | 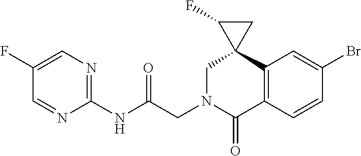 [SMILES: O=C(CN1C[C@]2(C[C@H]2F)c2cc(Br)ccc2C1=O)Nc1ncc(F)cn1] | 232 | 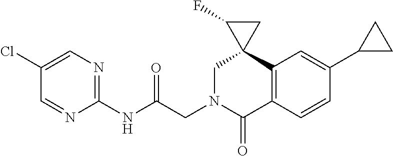 [SMILES: O=C(CN1C[C@]2(C[C@H]2F)c2cc(C3CC3)ccc2C1=O)Nc1ncc(Cl)cn1] |
| 109 | 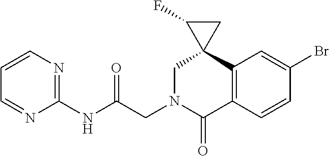 [SMILES: O=C(CN1C[C@]2(C[C@H]2F)c2cc(Br)ccc2C1=O)Nc1ncccn1] | 233 | 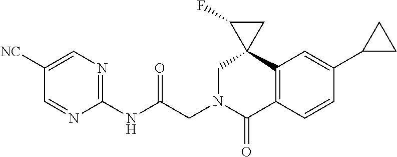 [SMILES: N#Cc1cnc(NC(=O)CN2C[C@]3(C[C@H]3F)c3cc(C4CC4)ccc3C2=O)nc1] |
| 110 | 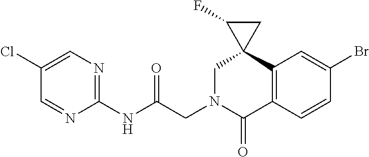 [SMILES: O=C(CN1C[C@]2(C[C@H]2F)c2cc(Br)ccc2C1=O)Nc1ncc(Cl)cn1] | 246 | 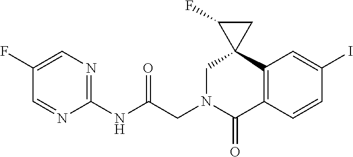 [SMILES: O=C(CN1C[C@]2(C[C@H]2F)c2cc(I)ccc2C1=O)Nc1ncc(F)cn1] |
| 247 | 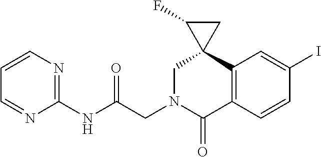 [SMILES: O=C(CN1C[C@]2(C[C@H]2F)c2cc(I)ccc2C1=O)Nc1ncccn1] | 273 | 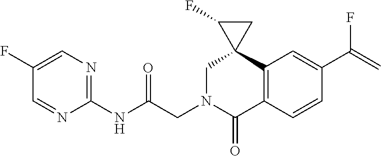 [SMILES: C=C(F)c1ccc2c(c1)[C@@]1(C[C@H]1F)CN(CC(=O)Nc1ncc(F)cn1)C2=O] |
| 248 | 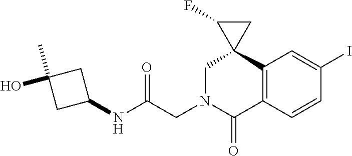 | 274 | 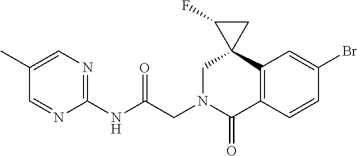 [SMILES: Cc1cnc(NC(=O)CN2C[C@]3(C[C@H]3F)c3cc(Br)ccc3C2=O)nc1] |
| 249 | 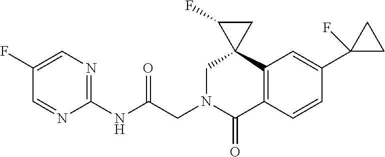 [SMILES: O=C(CN1C[C@]2(C[C@H]2F)c2cc(C3(F)CC3)ccc2C1=O)Nc1ncc(F)cn1] | 275 | 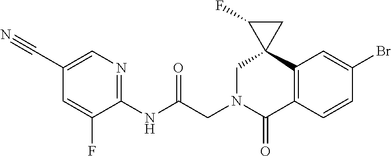 [SMILES: N#Cc1cnc(NC(=O)CN2C[C@]3(C[C@H]3F)c3cc(Br)ccc3C2=O)c(F)c1] |
| 250 | 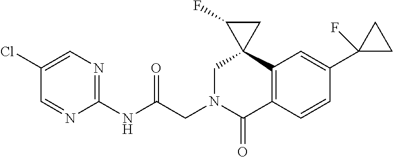 [SMILES: O=C(CN1C[C@]2(C[C@H]2F)c2cc(C3(F)CC3)ccc2C1=O)Nc1ncc(Cl)cn1] | 276 | 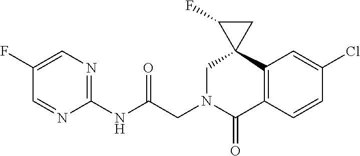 [SMILES: O=C(CN1C[C@]2(C[C@H]2F)c2cc(Cl)ccc2C1=O)Nc1ncc(F)cn1] |
| 271 | 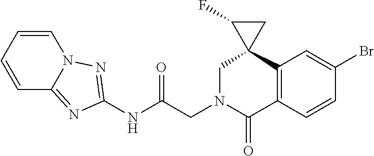 [SMILES: O=C(CN1C[C@]2(C[C@H]2F)c2cc(Br)ccc2C1=O)Nc1nc2ccccn2n1] | 308 | 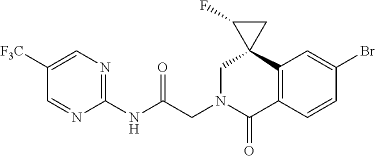 [SMILES: O=C(CN1C[C@]2(C[C@H]2F)c2cc(Br)ccc2C1=O)Nc1ncc(C(F)(F)F)cn1] |
| 272 | 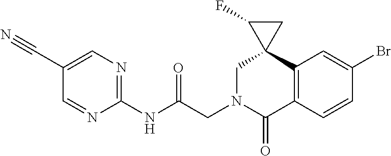 [SMILES: N#Cc1cnc(NC(=O)CN2C[C@]3(C[C@H]3F)c3cc(Br)ccc3C2=O)nc1] | 321 | 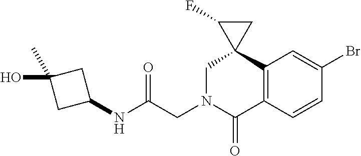 |

In certain embodiments, provided is a compound selected from Table 2 or a pharmaceutically acceptable salt, isotopically enriched analog, prodrug, stereoisomer, or a mixture of stereoisomers thereof:

TABLE 2

| Structure | Structure |
|---|---|
| 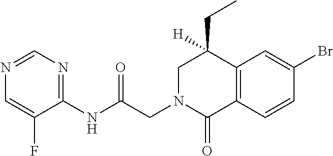 [SMILES: CC[C@@H]1CN(CC(=O)Nc2ncncc2F)C(=O)c2ccc(Br)cc21] | 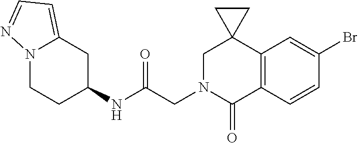 [SMILES: O=C(CN1CC2(CC2)c2cc(Br)ccc2C1=O)N[C@H]1CCn2nccc2C1] |
| 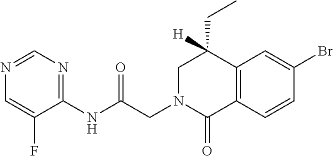 [SMILES: CC[C@H]1CN(CC(=O)Nc2ncncc2F)C(=O)c2ccc(Br)cc21] | 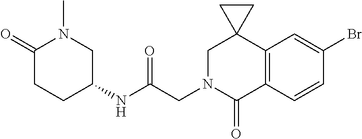 [SMILES: CN1C[C@H](NC(=O)CN2CC3(CC3)c3cc(Br)ccc3C2=O)CCC1=O] |
| 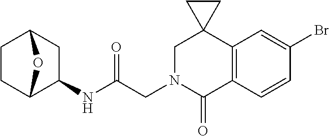 [SMILES: O=C(CN1CC2(CC2)c2cc(Br)ccc2C1=O)N[C@@H]1C[C@H]2CC[C@@H]1O2] | 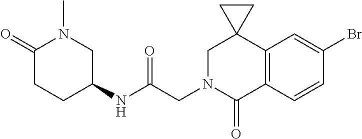 [SMILES: CN1C[C@@H](NC(=O)CN2CC3(CC3)c3cc(Br)ccc3C2=O)CCC1=O] |
| 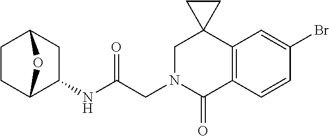 [SMILES: O=C(CN1CC2(CC2)c2cc(Br)ccc2C1=O)N[C@H]1C[C@H]2CC[C@@H]1O2] | 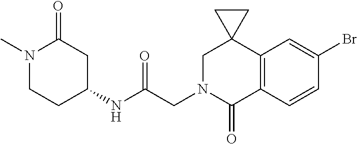 [SMILES: CN1CC[C@@H](NC(=O)CN2CC3(CC3)c3cc(Br)ccc3C2=O)CC1=O] |
| 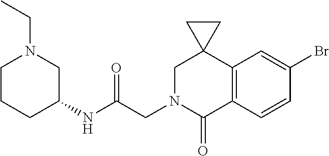 [SMILES: CCN1CCC[C@@H](NC(=O)CN2CC3(CC3)c3cc(Br)ccc3C2=O)C1] | 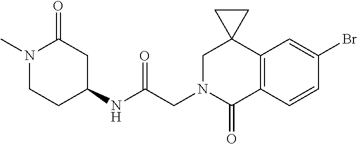 [SMILES: CN1CC[C@H](NC(=O)CN2CC3(CC3)c3cc(Br)ccc3C2=O)CC1=O] |
| 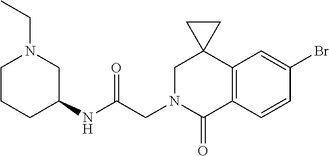 [SMILES: CCN1CCC[C@H](NC(=O)CN2CC3(CC3)c3cc(Br)ccc3C2=O)C1] | 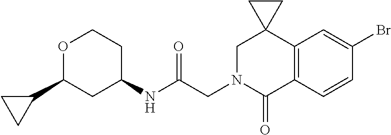 [SMILES: O=C(CN1CC2(CC2)c2cc(Br)ccc2C1=O)N[C@@H]1CCO[C@H](C2CC2)C1] |
| 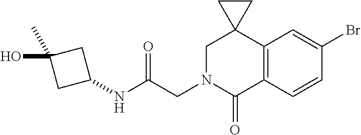 | 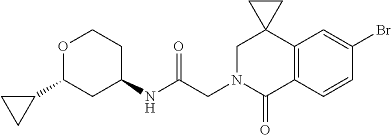 [SMILES: O=C(CN1CC2(CC2)c2cc(Br)ccc2C1=O)N[C@@H]1CCO[C@@H](C2CC2)C1] |
| 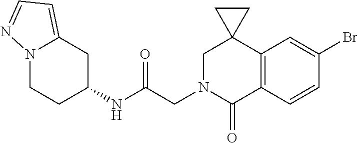 [SMILES: O=C(CN1CC2(CC2)c2cc(Br)ccc2C1=O)N[C@@H]1CCn2nccc2C1] | 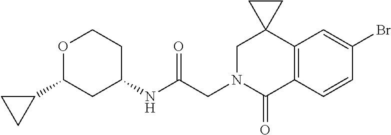 [SMILES: O=C(CN1CC2(CC2)c2cc(Br)ccc2C1=O)N[C@H]1CCO[C@@H](C2CC2)C1] |
| 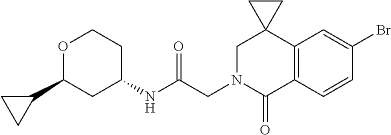 [SMILES: O=C(CN1CC2(CC2)c2cc(Br)ccc2C1=O)N[C@H]1CCO[C@H](C2CC2)C1] | 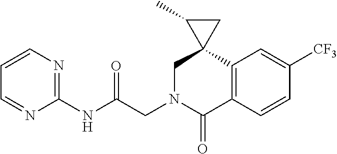 [SMILES: C[C@@H]1C[C@@]12CN(CC(=O)Nc1ncccn1)C(=O)c1ccc(C(F)(F)F)cc12] |
| 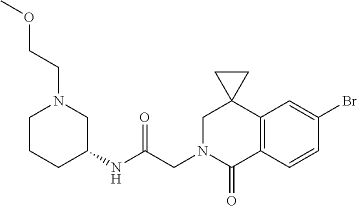 [SMILES: COCCN1CCC[C@@H](NC(=O)CN2CC3(CC3)c3cc(Br)ccc3C2=O)C1] | 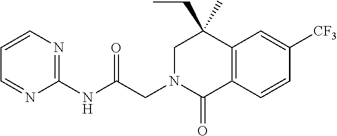 [SMILES: CC[C@]1(C)CN(CC(=O)Nc2ncccn2)C(=O)c2ccc(C(F)(F)F)cc21] |
| 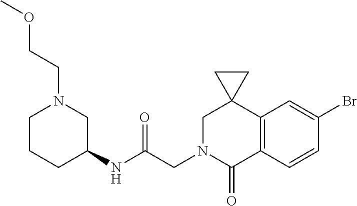 [SMILES: COCCN1CCC[C@H](NC(=O)CN2CC3(CC3)c3cc(Br)ccc3C2=O)C1] | 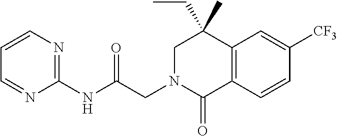 [SMILES: CC[C@@]1(C)CN(CC(=O)Nc2ncccn2)C(=O)c2ccc(C(F)(F)F)cc21] |
| 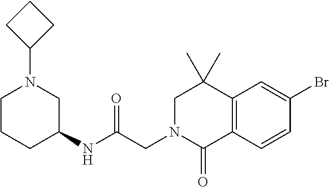 [SMILES: CC1(C)CN(CC(=O)N[C@H]2CCCN(C3CCC3)C2)C(=O)c2ccc(Br)cc21] | 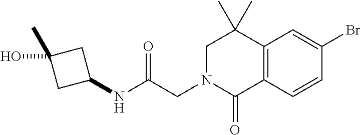 |
| 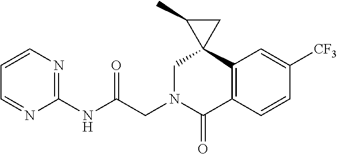 [SMILES: C[C@H]1C[C@]12CN(CC(=O)Nc1ncccn1)C(=O)c1ccc(C(F)(F)F)cc12] | 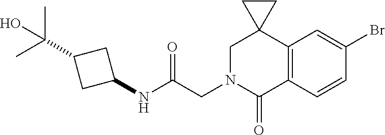 |
| 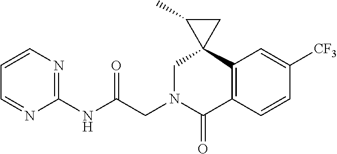 [SMILES: C[C@@H]1C[C@]12CN(CC(=O)Nc1ncccn1)C(=O)c1ccc(C(F)(F)F)cc12] | 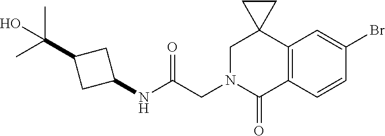 |
| 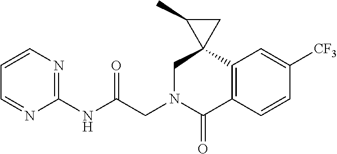 [SMILES: C[C@H]1C[C@@]12CN(CC(=O)Nc1ncccn1)C(=O)c1ccc(C(F)(F)F)cc12] | 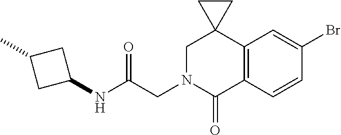 [SMILES: C[C@H]1C[C@H](NC(=O)CN2CC3(CC3)c3cc(Br)ccc3C2=O)C1] |
| | 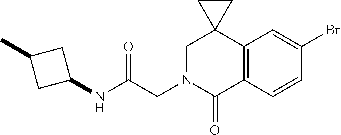 [SMILES: C[C@H]1C[C@@H](NC(=O)CN2CC3(CC3)c3cc(Br)ccc3C2=O)C1] |
| 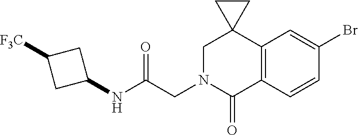 [SMILES: O=C(CN1CC2(CC2)c2cc(Br)ccc2C1=O)N[C@H]1C[C@@H](C(F)(F)F)C1] | 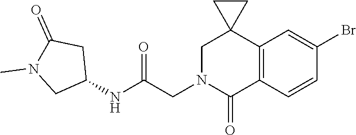 [SMILES: CN1C[C@@H](NC(=O)CN2CC3(CC3)c3cc(Br)ccc3C2=O)CC1=O] |
| 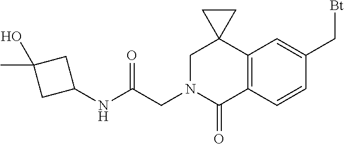 | 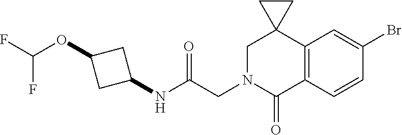 [SMILES: O=C(CN1CC2(CC2)c2cc(Br)ccc2C1=O)N[C@H]1C[C@@H](OC(F)F)C1] |
| 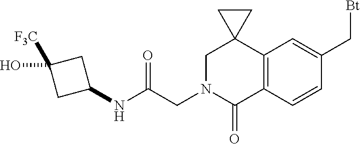 | 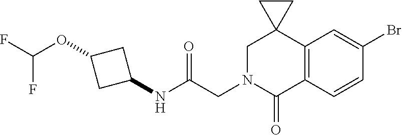 [SMILES: O=C(CN1CC2(CC2)c2cc(Br)ccc2C1=O)N[C@H]1C[C@H](OC(F)F)C1] |
| 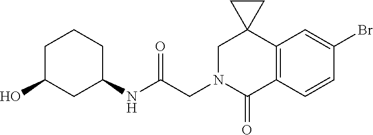 [SMILES: O=C(CN1CC2(CC2)c2cc(Br)ccc2C1=O)N[C@@H]1CCC[C@H](O)C1] | 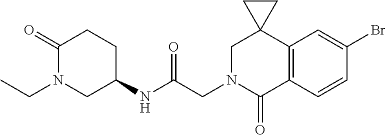 [SMILES: CCN1C[C@H](NC(=O)CN2CC3(CC3)c3cc(Br)ccc3C2=O)CCC1=O] |
| 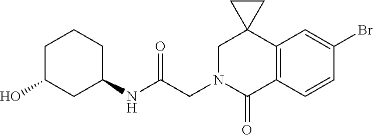 [SMILES: O=C(CN1CC2(CC2)c2cc(Br)ccc2C1=O)N[C@@H]1CCC[C@@H](O)C1] | 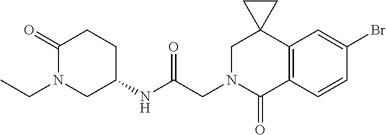 [SMILES: CCN1C[C@@H](NC(=O)CN2CC3(CC3)c3cc(Br)ccc3C2=O)CCC1=O] |
| 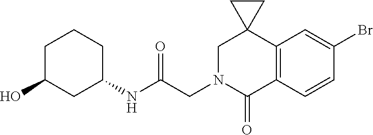 [SMILES: O=C(CN1CC2(CC2)c2cc(Br)ccc2C1=O)N[C@H]1CCC[C@H](O)C1] | 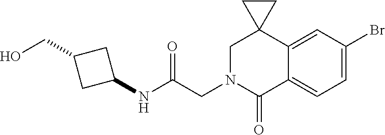 [SMILES: O=C(CN1CC2(CC2)c2cc(Br)ccc2C1=O)N[C@H]1C[C@H](CO)C1] |
| 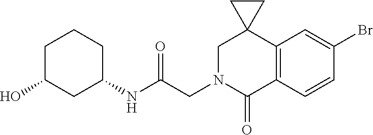 [SMILES: O=C(CN1CC2(CC2)c2cc(Br)ccc2C1=O)N[C@H]1CCC[C@@H](O)C1] | 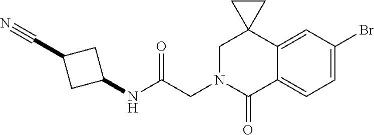 |
| 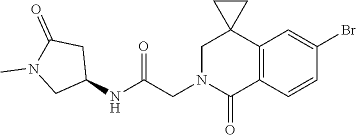 [SMILES: CN1C[C@H](NC(=O)CN2CC3(CC3)c3cc(Br)ccc3C2=O)CC1=O] | 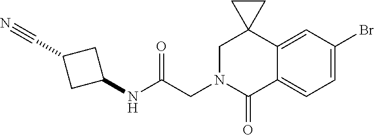 |
| 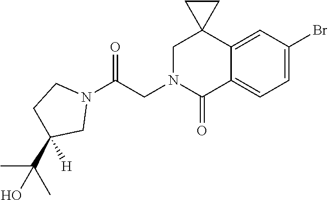 [SMILES: CC(C)(O)[C@H]1CCN(C(=O)CN2CC3(CC3)c3cc(Br)ccc3C2=O)C1] | 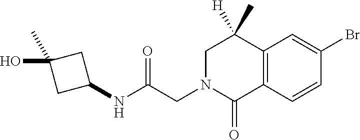 [SMILES: C[C@@H]1CN(CC(=O)N[C@H]2C[C@@](C)(O)C2)C(=O)c2ccc(Br)cc21] |
| 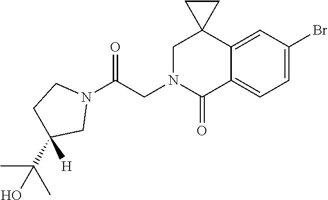 [SMILES: CC(C)(O)[C@@H]1CCN(C(=O)CN2CC3(CC3)c3cc(Br)ccc3C2=O)C1] | 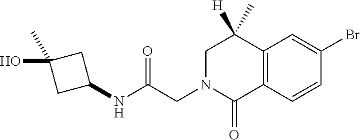 [SMILES: C[C@H]1CN(CC(=O)N[C@H]2C[C@@](C)(O)C2)C(=O)c2ccc(Br)cc21] |
| 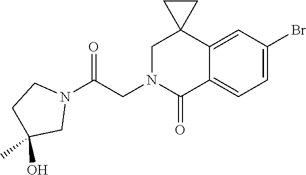 [SMILES: C[C@]1(O)CCN(C(=O)CN2CC3(CC3)c3cc(Br)ccc3C2=O)C1] | 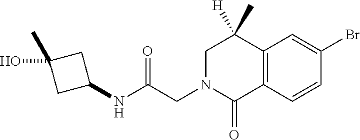 [SMILES: C[C@@H]1CN(CC(=O)N[C@H]2C[C@](C)(O)C2)C(=O)c2ccc(Br)cc21] |
| | 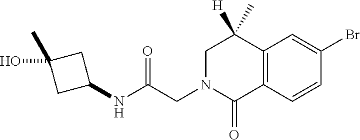 [SMILES: C[C@H]1CN(CC(=O)N[C@H]2C[C@](C)(O)C2)C(=O)c2ccc(Br)cc21] |
| 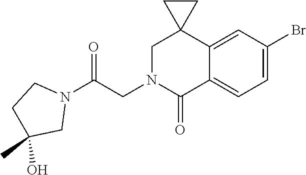 [SMILES: C[C@@]1(O)CCN(C(=O)CN2CC3(CC3)c3cc(Br)ccc3C2=O)C1] | 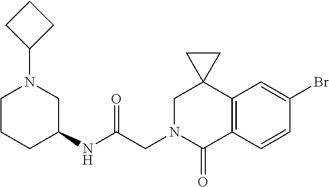 [SMILES: O=C(CN1CC2(CC2)c2cc(Br)ccc2C1=O)N[C@H]1CCCN(C2CCC2)C1] |
| 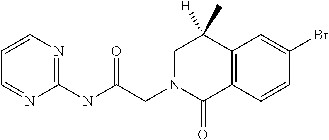 | 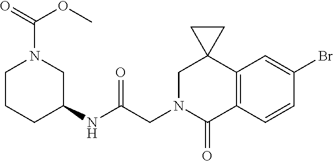 [SMILES: COC(=O)N1CCC[C@H](NC(=O)CN2CC3(CC3)c3cc(Br)ccc3C2=O)C1] |
| 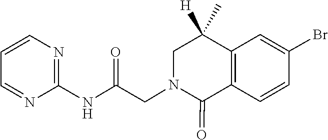 [SMILES: C[C@H]1CN(CC(=O)Nc2ncccn2)C(=O)c2ccc(Br)cc21] | 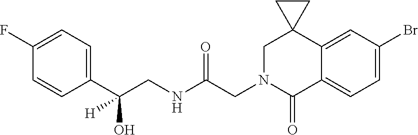 [SMILES: O=C(CN1CC2(CC2)c2cc(Br)ccc2C1=O)NC[C@@H](O)c1ccc(F)cc1] |
| 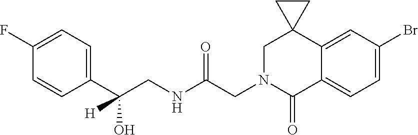 [SMILES: O=C(CN1CC2(CC2)c2cc(Br)ccc2C1=O)NC[C@H](O)c1ccc(F)cc1] | 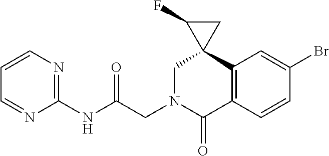 [SMILES: O=C(CN1C[C@]2(C[C@@H]2F)c2cc(Br)ccc2C1=O)Nc1ncccn1] |
| 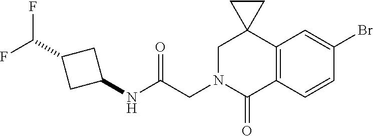 | 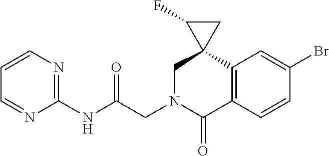 [SMILES: O=C(CN1C[C@@]2(C[C@H]2F)c2cc(Br)ccc2C1=O)Nc1ncccn1] |
| 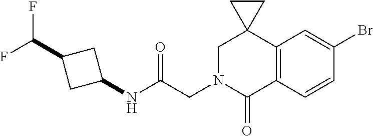 | 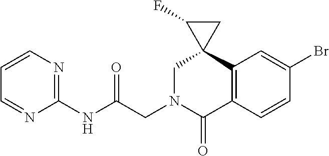 [SMILES: O=C(CN1C[C@]2(C[C@H]2F)c2cc(Br)ccc2C1=O)Nc1ncccn1] |
| 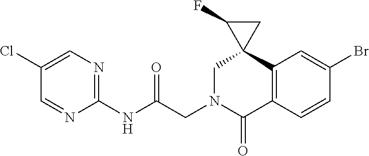 [SMILES: O=C(CN1C[C@]2(C[C@@H]2F)c2cc(Br)ccc2C1=O)Nc1ncc(Cl)cn1] | 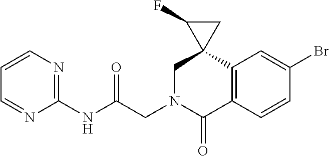 [SMILES: O=C(CN1C[C@@]2(C[C@@H]2F)c2cc(Br)ccc2C1=O)Nc1ncccn1] |
| 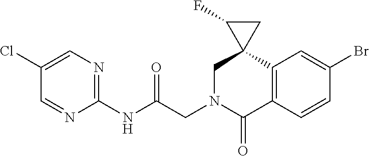 [SMILES: O=C(CN1C[C@@]2(C[C@H]2F)c2cc(Br)ccc2C1=O)Nc1ncc(Cl)cn1] | 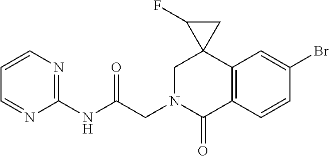 [SMILES: O=C(CN1CC2(CC2F)c2cc(Br)ccc2C1=O)Nc1ncccn1] |
| 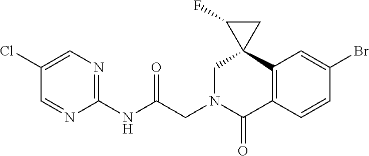 [SMILES: O=C(CN1C[C@]2(C[C@H]2F)c2cc(Br)ccc2C1=O)Nc1ncc(Cl)cn1] | 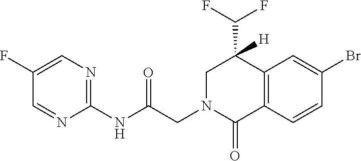 [SMILES: O=C(CN1C[C@H](C(F)F)c2cc(Br)ccc2C1=O)Nc1ncc(F)cn1] |
| 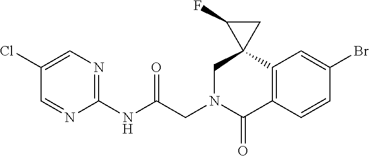 [SMILES: O=C(CN1C[C@@]2(C[C@@H]2F)c2cc(Br)ccc2C1=O)Nc1ncc(Cl)cn1] | 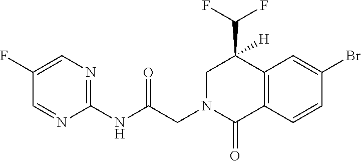 [SMILES: O=C(CN1C[C@@H](C(F)F)c2cc(Br)ccc2C1=O)Nc1ncc(F)cn1] |
| 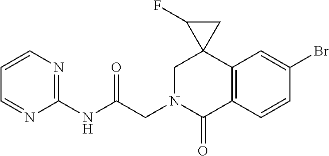 [SMILES: O=C(CN1CC2(CC2F)c2cc(Br)ccc2C1=O)Nc1ncccn1] | |
| 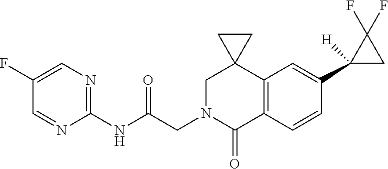 [SMILES: O=C(CN1CC2(CC2)c2cc([C@@H]3CC3(F)F)ccc2C1=O)Nc1ncc(F)cn1] | 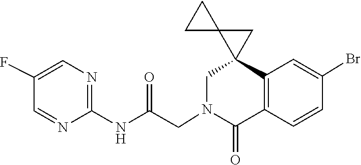 [SMILES: O=C(CN1C[C@]2(CC23CC3)c2cc(Br)ccc2C1=O)Nc1ncc(F)cn1] |
| 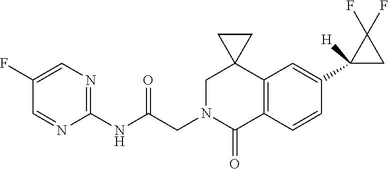 [SMILES: O=C(CN1CC2(CC2)c2cc([C@H]3CC3(F)F)ccc2C1=O)Nc1ncc(F)cn1] | 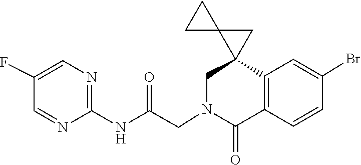 [SMILES: O=C(CN1C[C@@]2(CC23CC3)c2cc(Br)ccc2C1=O)Nc1ncc(F)cn1] |
| 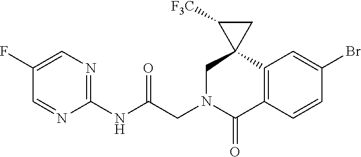 [SMILES: O=C(CN1C[C@@]2(C[C@H]2C(F)(F)F)c2cc(Br)ccc2C1=O)Nc1ncc(F)cn1] | 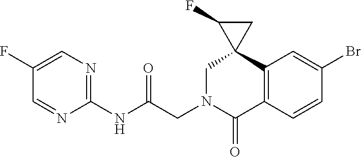 [SMILES: O=C(CN1C[C@]2(C[C@@H]2F)c2cc(Br)ccc2C1=O)Nc1ncc(F)cn1] |
| 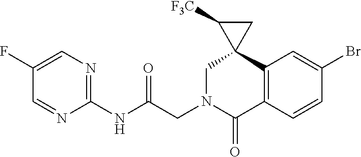 [SMILES: O=C(CN1C[C@]2(C[C@@H]2C(F)(F)F)c2cc(Br)ccc2C1=O)Nc1ncc(F)cn1] | 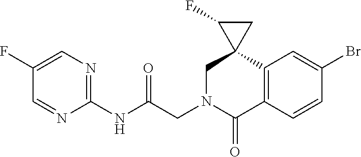 [SMILES: O=C(CN1C[C@@]2(C[C@H]2F)c2cc(Br)ccc2C1=O)Nc1ncc(F)cn1] |
| 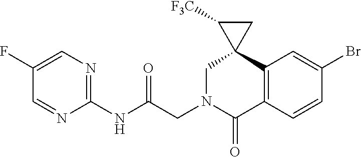 [SMILES: O=C(CN1C[C@]2(C[C@H]2C(F)(F)F)c2cc(Br)ccc2C1=O)Nc1ncc(F)cn1] | 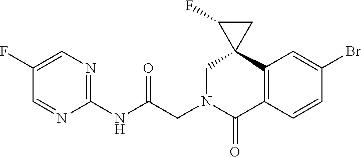 [SMILES: O=C(CN1C[C@]2(C[C@H]2F)c2cc(Br)ccc2C1=O)Nc1ncc(F)cn1] |
| 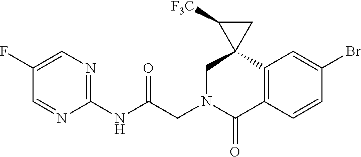 [SMILES: O=C(CN1C[C@@]2(C[C@@H]2C(F)(F)F)c2cc(Br)ccc2C1=O)Nc1ncc(F)cn1] | 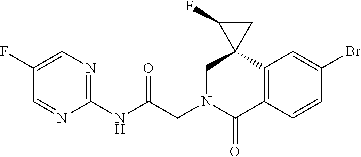 [SMILES: O=C(CN1C[C@@]2(C[C@@H]2F)c2cc(Br)ccc2C1=O)Nc1ncc(F)cn1] |
| 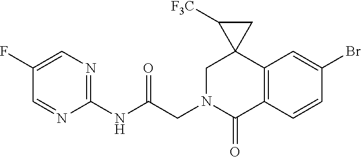 [SMILES: O=C(CN1CC2(CC2C(F)(F)F)c2cc(Br)ccc2C1=O)Nc1ncc(F)cn1] | 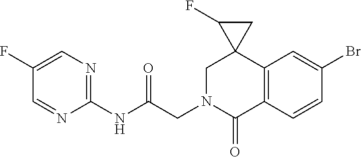 [SMILES: O=C(CN1CC2(CC2F)c2cc(Br)ccc2C1=O)Nc1ncc(F)cn1] |
| | 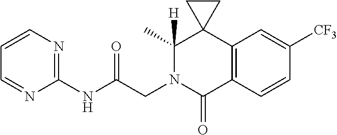 [SMILES: C[C@H]1N(CC(=O)Nc2ncccn2)C(=O)c2ccc(C(F)(F)F)cc2C12CC2] |
| 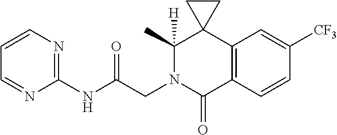 [SMILES: C[C@@H]1N(CC(=O)Nc2ncccn2)C(=O)c2ccc(C(F)(F)F)cc2C12CC2] | 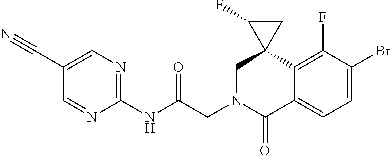 [SMILES: N#Cc1cnc(NC(=O)CN2C[C@@]3(C[C@H]3F)c3c(ccc(Br)c3F)C2=O)nc1] |
| 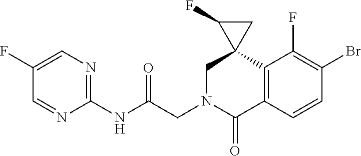 [SMILES: O=C(CN1C[C@@]2(C[C@@H]2F)c2c(ccc(Br)c2F)C1=O)Nc1ncc(F)cn1] | 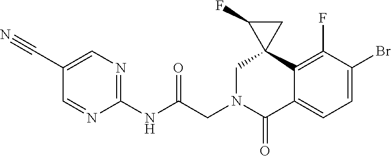 [SMILES: N#Cc1cnc(NC(=O)CN2C[C@]3(C[C@@H]3F)c3c(ccc(Br)c3F)C2=O)nc1] |
| 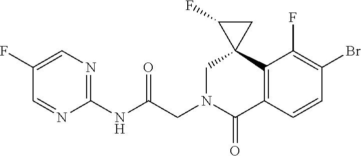 [SMILES: O=C(CN1C[C@]2(C[C@H]2F)c2c(ccc(Br)c2F)C1=O)Nc1ncc(F)cn1] | 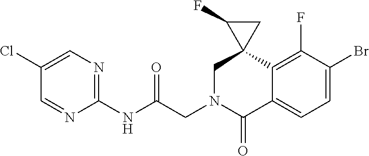 [SMILES: O=C(CN1C[C@@]2(C[C@@H]2F)c2c(ccc(Br)c2F)C1=O)Nc1ncc(Cl)cn1] |
| 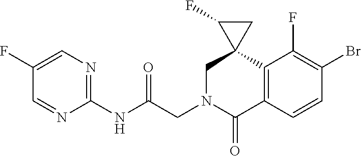 [SMILES: O=C(CN1C[C@@]2(C[C@H]2F)c2c(ccc(Br)c2F)C1=O)Nc1ncc(F)cn1] | 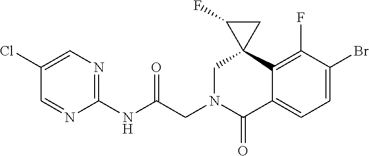 [SMILES: O=C(CN1C[C@]2(C[C@H]2F)c2c(ccc(Br)c2F)C1=O)Nc1ncc(Cl)cn1] |
| 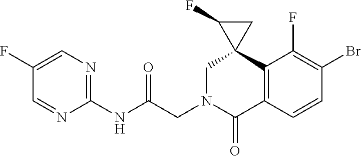 [SMILES: O=C(CN1C[C@]2(C[C@@H]2F)c2c(ccc(Br)c2F)C1=O)Nc1ncc(F)cn1] | 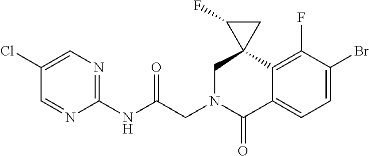 [SMILES: O=C(CN1C[C@@]2(C[C@H]2F)c2c(ccc(Br)c2F)C1=O)Nc1ncc(Cl)cn1] |
| 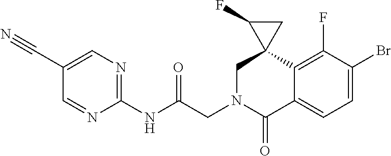 [SMILES: N#Cc1cnc(NC(=O)CN2C[C@@]3(C[C@@H]3F)c3c(ccc(Br)c3F)C2=O)nc1] | 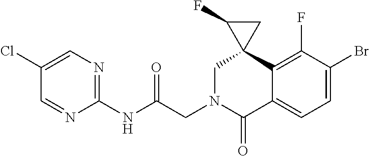 [SMILES: O=C(CN1C[C@]2(C[C@@H]2F)c2c(ccc(Br)c2F)C1=O)Nc1ncc(Cl)cn1] |
| 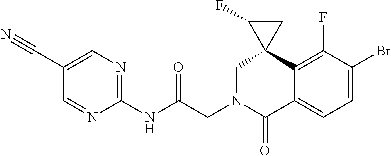 [SMILES: N#Cc1cnc(NC(=O)CN2C[C@]3(C[C@H]3F)c3c(ccc(Br)c3F)C2=O)nc1] | 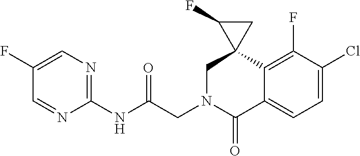 [SMILES: O=C(CN1C[C@@]2(C[C@@H]2F)c2c(ccc(Cl)c2F)C1=O)Nc1ncc(F)cn1] |
| 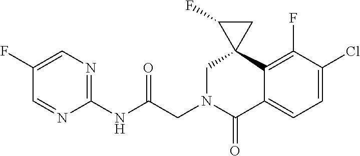 [SMILES: O=C(CN1C[C@]2(C[C@H]2F)c2c(ccc(Cl)c2F)C1=O)Nc1ncc(F)cn1] | 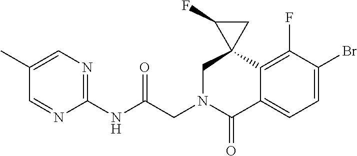 [SMILES: Cc1cnc(NC(=O)CN2C[C@@]3(C[C@@H]3F)c3c(ccc(Br)c3F)C2=O)nc1] |
| 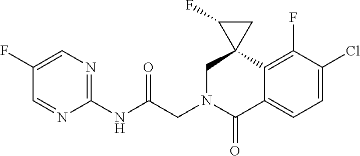 [SMILES: O=C(CN1C[C@@]2(C[C@H]2F)c2c(ccc(Cl)c2F)C1=O)Nc1ncc(F)cn1] | 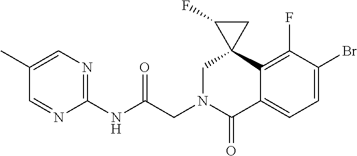 [SMILES: Cc1cnc(NC(=O)CN2C[C@]3(C[C@H]3F)c3c(ccc(Br)c3F)C2=O)nc1] |
| 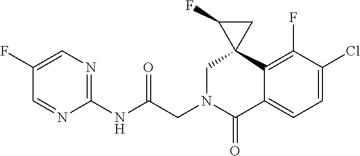 [SMILES: O=C(CN1C[C@]2(C[C@@H]2F)c2c(ccc(Cl)c2F)C1=O)Nc1ncc(F)cn1] | 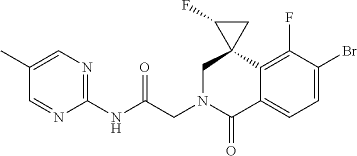 [SMILES: Cc1cnc(NC(=O)CN2C[C@@]3(C[C@H]3F)c3c(ccc(Br)c3F)C2=O)nc1] |
| 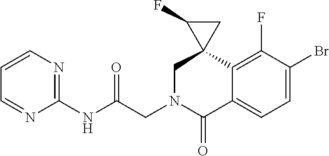 [SMILES: O=C(CN1C[C@@]2(C[C@@H]2F)c2c(ccc(Br)c2F)C1=O)Nc1ncccn1] | 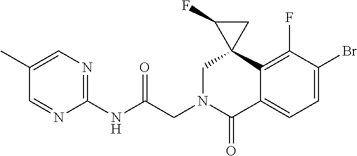 [SMILES: Cc1cnc(NC(=O)CN2C[C@]3(C[C@@H]3F)c3c(ccc(Br)c3F)C2=O)nc1] |
| 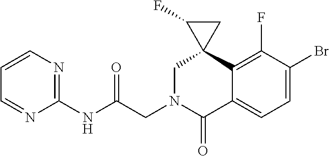 [SMILES: O=C(CN1C[C@]2(C[C@H]2F)c2c(ccc(Br)c2F)C1=O)Nc1ncccn1] | 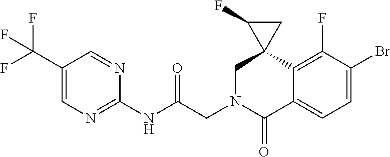 [SMILES: O=C(CN1C[C@@]2(C[C@@H]2F)c2c(ccc(Br)c2F)C1=O)Nc1ncc(C(F)(F)F)cn1] |
| 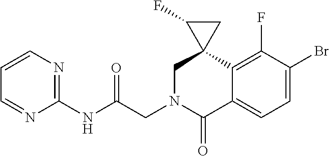 [SMILES: O=C(CN1C[C@@]2(C[C@H]2F)c2c(ccc(Br)c2F)C1=O)Nc1ncccn1] | 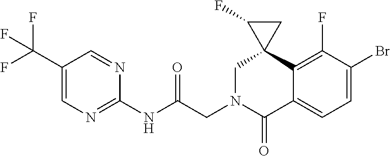 [SMILES: O=C(CN1C[C@]2(C[C@H]2F)c2c(ccc(Br)c2F)C1=O)Nc1ncc(C(F)(F)F)cn1] |
| 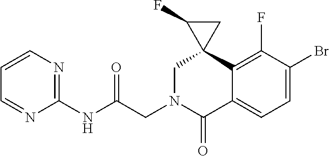 [SMILES: O=C(CN1C[C@]2(C[C@@H]2F)c2c(ccc(Br)c2F)C1=O)Nc1ncccn1] | 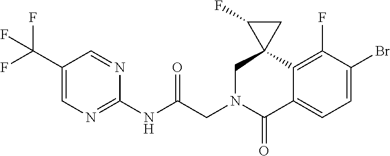 [SMILES: O=C(CN1C[C@@]2(C[C@H]2F)c2c(ccc(Br)c2F)C1=O)Nc1ncc(C(F)(F)F)cn1] |
| 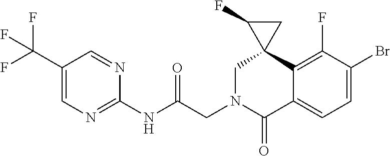 [SMILES: O=C(CN1C[C@]2(C[C@@H]2F)c2c(ccc(Br)c2F)C1=O)Nc1ncc(C(F)(F)F)cn1] | 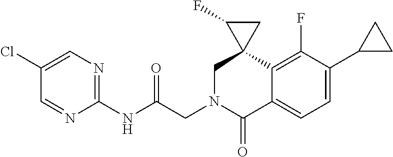 [SMILES: O=C(CN1C[C@@]2(C[C@H]2F)c2c(ccc(C3CC3)c2F)C1=O)Nc1ncc(Cl)cn1] |
| 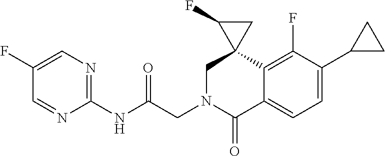 [SMILES: O=C(CN1C[C@@]2(C[C@@H]2F)c2c(ccc(C3CC3)c2F)C1=O)Nc1ncc(F)cn1] | 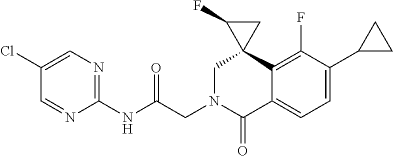 [SMILES: O=C(CN1C[C@]2(C[C@@H]2F)c2c(ccc(C3CC3)c2F)C1=O)Nc1ncc(Cl)cn1] |
| 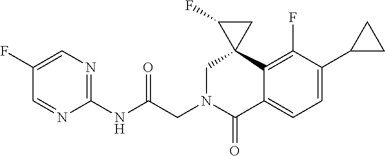 [SMILES: O=C(CN1C[C@]2(C[C@H]2F)c2c(ccc(C3CC3)c2F)C1=O)Nc1ncc(F)cn1] | 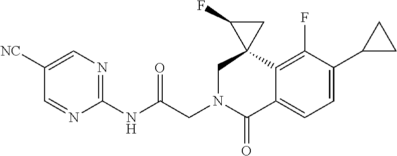 [SMILES: N#Cc1cnc(NC(=O)CN2C[C@@]3(C[C@@H]3F)c3c(ccc(C4CC4)c3F)C2=O)nc1] |
| 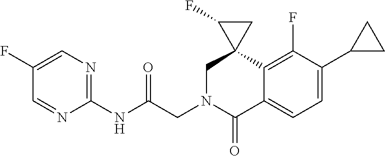 [SMILES: O=C(CN1C[C@@]2(C[C@H]2F)c2c(ccc(C3CC3)c2F)C1=O)Nc1ncc(F)cn1] | 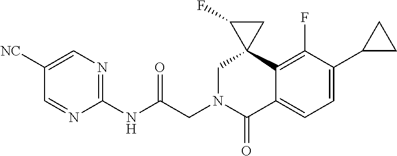 [SMILES: N#Cc1cnc(NC(=O)CN2C[C@]3(C[C@H]3F)c3c(ccc(C4CC4)c3F)C2=O)nc1] |
| 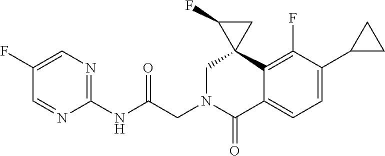 [SMILES: O=C(CN1C[C@]2(C[C@@H]2F)c2c(ccc(C3CC3)c2F)C1=O)Nc1ncc(F)cn1] | 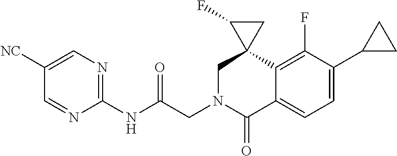 [SMILES: N#Cc1cnc(NC(=O)CN2C[C@@]3(C[C@H]3F)c3c(ccc(C4CC4)c3F)C2=O)nc1] |
| 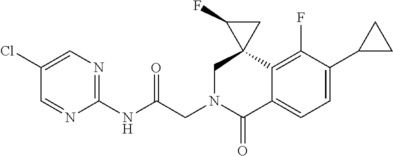 [SMILES: O=C(CN1C[C@@]2(C[C@@H]2F)c2c(ccc(C3CC3)c2F)C1=O)Nc1ncc(Cl)cn1] | 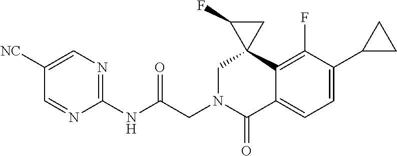 [SMILES: N#Cc1cnc(NC(=O)CN2C[C@]3(C[C@@H]3F)c3c(ccc(C4CC4)c3F)C2=O)nc1] |
| 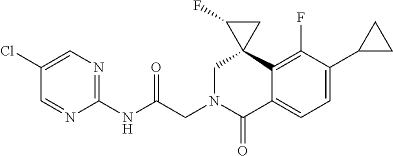 [SMILES: O=C(CN1C[C@]2(C[C@H]2F)c2c(ccc(C3CC3)c2F)C1=O)Nc1ncc(Cl)cn1] | 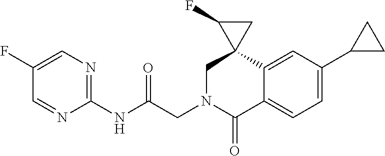 [SMILES: O=C(CN1C[C@@]2(C[C@@H]2F)c2cc(C3CC3)ccc2C1=O)Nc1ncc(F)cn1] |
| 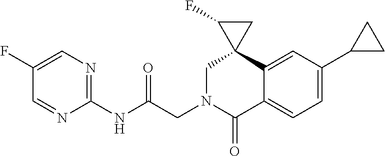 [SMILES: O=C(CN1C[C@]2(C[C@H]2F)c2cc(C3CC3)ccc2C1=O)Nc1ncc(F)cn1] | 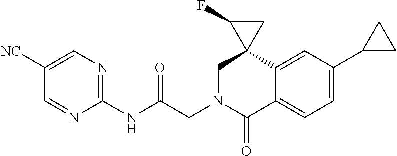 [SMILES: N#Cc1cnc(NC(=O)CN2C[C@@]3(C[C@@H]3F)c3cc(C4CC4)ccc3C2=O)nc1] |
| 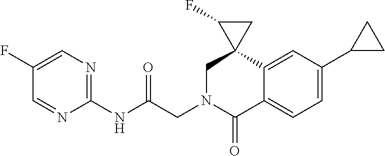 [SMILES: O=C(CN1C[C@@]2(C[C@H]2F)c2cc(C3CC3)ccc2C1=O)Nc1ncc(F)cn1] | 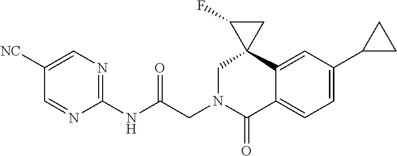 [SMILES: N#Cc1cnc(NC(=O)CN2C[C@]3(C[C@H]3F)c3cc(C4CC4)ccc3C2=O)nc1] |
| 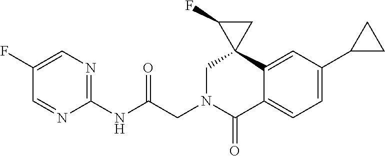 [SMILES: O=C(CN1C[C@]2(C[C@@H]2F)c2cc(C3CC3)ccc2C1=O)Nc1ncc(F)cn1] | 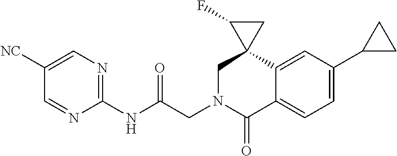 [SMILES: N#Cc1cnc(NC(=O)CN2C[C@@]3(C[C@H]3F)c3cc(C4CC4)ccc3C2=O)nc1] |
| 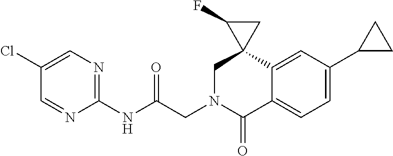 [SMILES: O=C(CN1C[C@@]2(C[C@@H]2F)c2cc(C3CC3)ccc2C1=O)Nc1ncc(Cl)cn1] | 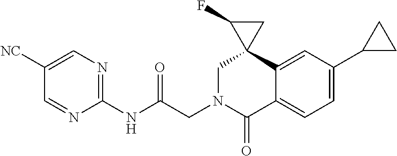 [SMILES: N#Cc1cnc(NC(=O)CN2C[C@]3(C[C@@H]3F)c3cc(C4CC4)ccc3C2=O)nc1] |
| 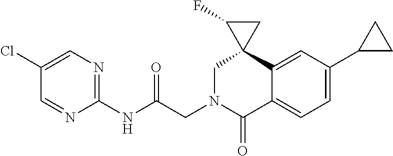 [SMILES: O=C(CN1C[C@]2(C[C@H]2F)c2cc(C3CC3)ccc2C1=O)Nc1ncc(Cl)cn1] | 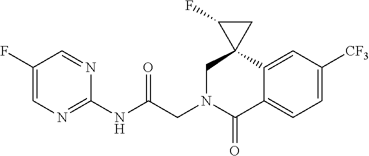 [SMILES: O=C(CN1C[C@@]2(C[C@H]2F)c2cc(C(F)(F)F)ccc2C1=O)Nc1ncc(F)cn1] |
| 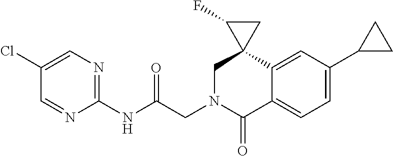 [SMILES: O=C(CN1C[C@@]2(C[C@H]2F)c2cc(C3CC3)ccc2C1=O)Nc1ncc(Cl)cn1] | 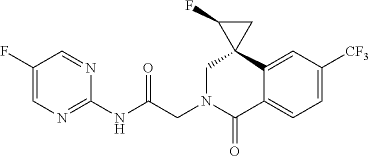 [SMILES: O=C(CN1C[C@]2(C[C@@H]2F)c2cc(C(F)(F)F)ccc2C1=O)Nc1ncc(F)cn1] |
| 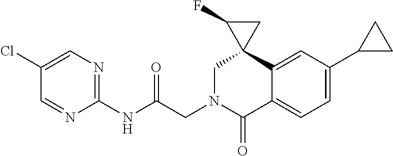 [SMILES: O=C(CN1C[C@]2(C[C@@H]2F)c2cc(C3CC3)ccc2C1=O)Nc1ncc(Cl)cn1] | 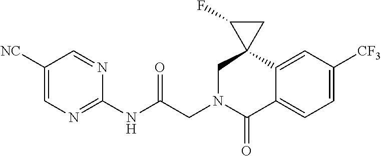 [SMILES: N#Cc1cnc(NC(=O)CN2C[C@@]3(C[C@H]3F)c3cc(C(F)(F)F)ccc3C2=O)nc1] |
| 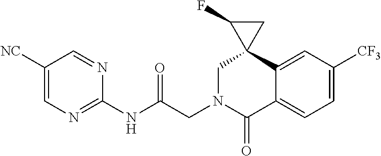 [SMILES: N#Cc1cnc(NC(=O)CN2C[C@]3(C[C@@H]3F)c3cc(C(F)(F)F)ccc3C2=O)nc1] | 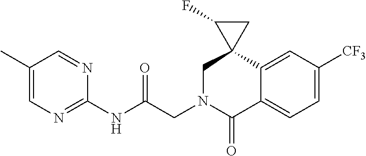 [SMILES: Cc1cnc(NC(=O)CN2C[C@@]3(C[C@H]3F)c3cc(C(F)(F)F)ccc3C2=O)nc1] |
| 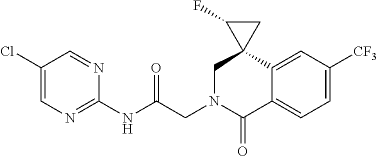 [SMILES: O=C(CN1C[C@@]2(C[C@H]2F)c2cc(C(F)(F)F)ccc2C1=O)Nc1ncc(Cl)cn1] | 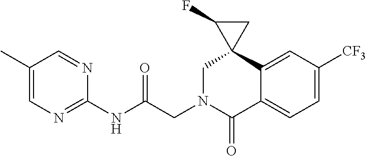 [SMILES: Cc1cnc(NC(=O)CN2C[C@]3(C[C@@H]3F)c3cc(C(F)(F)F)ccc3C2=O)nc1] |
| 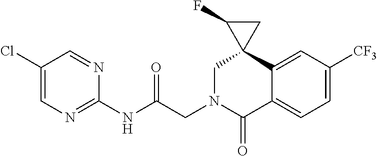 [SMILES: O=C(CN1C[C@]2(C[C@@H]2F)c2cc(C(F)(F)F)ccc2C1=O)Nc1ncc(Cl)cn1] | 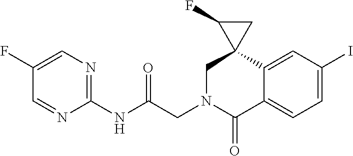 [SMILES: O=C(CN1C[C@@]2(C[C@@H]2F)c2cc(I)ccc2C1=O)Nc1ncc(F)cn1] |
| 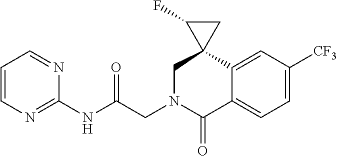 [SMILES: O=C(CN1C[C@@]2(C[C@H]2F)c2cc(C(F)(F)F)ccc2C1=O)Nc1ncccn1] | 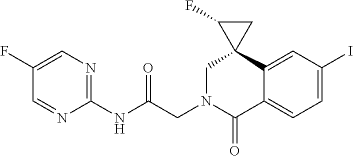 [SMILES: O=C(CN1C[C@]2(C[C@H]2F)c2cc(I)ccc2C1=O)Nc1ncc(F)cn1] |
| 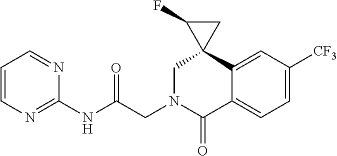 [SMILES: O=C(CN1C[C@]2(C[C@@H]2F)c2cc(C(F)(F)F)ccc2C1=O)Nc1ncccn1] | 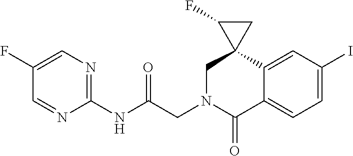 [SMILES: O=C(CN1C[C@@]2(C[C@H]2F)c2cc(I)ccc2C1=O)Nc1ncc(F)cn1] |
| 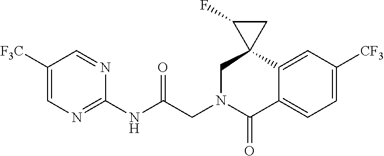 [SMILES: O=C(CN1C[C@@]2(C[C@H]2F)c2cc(C(F)(F)F)ccc2C1=O)Nc1ncc(C(F)(F)F)cn1] | 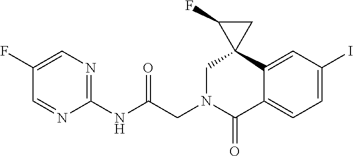 [SMILES: O=C(CN1C[C@]2(C[C@@H]2F)c2cc(I)ccc2C1=O)Nc1ncc(F)cn1] |
| 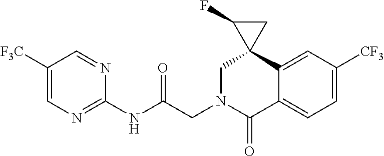 [SMILES: O=C(CN1C[C@]2(C[C@@H]2F)c2cc(C(F)(F)F)ccc2C1=O)Nc1ncc(C(F)(F)F)cn1] | 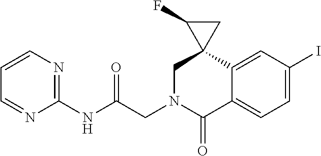 [SMILES: O=C(CN1C[C@@]2(C[C@@H]2F)c2cc(I)ccc2C1=O)Nc1ncccn1] |
| 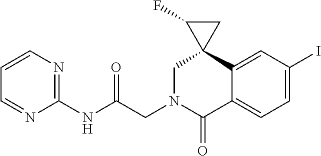 [SMILES: O=C(CN1C[C@]2(C[C@H]2F)c2cc(I)ccc2C1=O)Nc1ncccn1] | 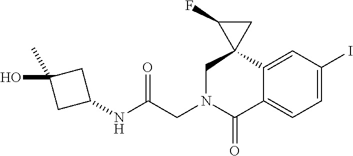 |
| 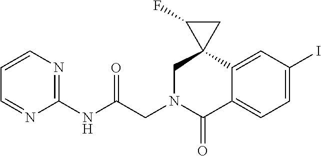 [SMILES: O=C(CN1C[C@@]2(C[C@H]2F)c2cc(I)ccc2C1=O)Nc1ncccn1] | 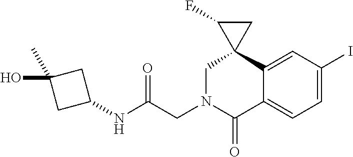 |
| 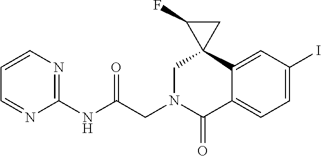 [SMILES: O=C(CN1C[C@]2(C[C@@H]2F)c2cc(I)ccc2C1=O)Nc1ncccn1] | 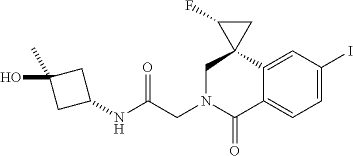 |
| 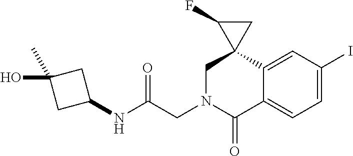 | 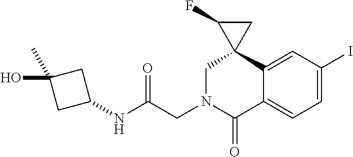 |
| 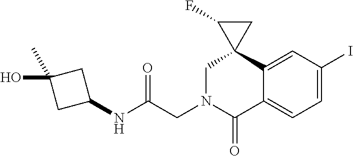 | 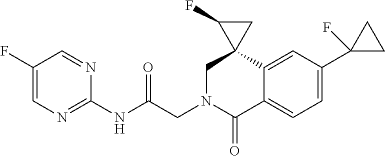 [SMILES: O=C(CN1C[C@@]2(C[C@@H]2F)c2cc(C3(F)CC3)ccc2C1=O)Nc1ncc(F)cn1] |
| 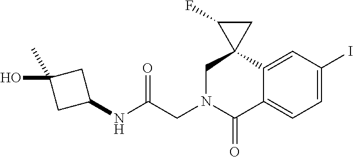 | 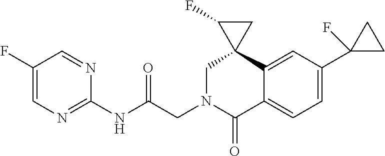 [SMILES: O=C(CN1C[C@]2(C[C@H]2F)c2cc(C3(F)CC3)ccc2C1=O)Nc1ncc(F)cn1] |
| 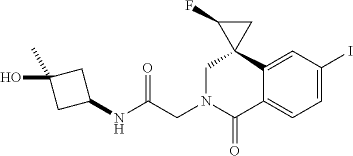 | 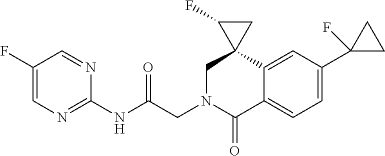 [SMILES: O=C(CN1C[C@@]2(C[C@H]2F)c2cc(C3(F)CC3)ccc2C1=O)Nc1ncc(F)cn1] |
| 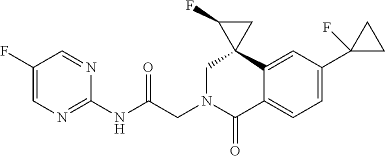 [SMILES: O=C(CN1C[C@]2(C[C@@H]2F)c2cc(C3(F)CC3)ccc2C1=O)Nc1ncc(F)cn1] | 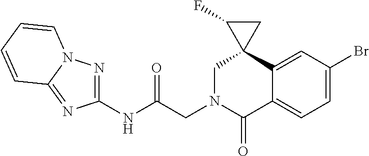 [SMILES: O=C(CN1C[C@]2(C[C@H]2F)c2cc(Br)ccc2C1=O)Nc1nc2ccccn2n1] |
| 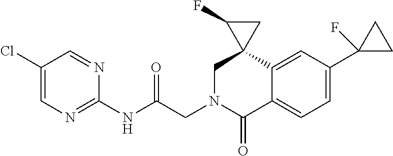 [SMILES: O=C(CN1C[C@@]2(C[C@@H]2F)c2cc(C3(F)CC3)ccc2C1=O)Nc1ncc(Cl)cn1] | 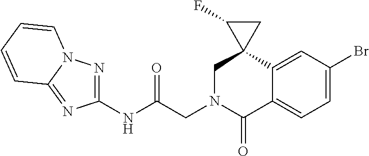 [SMILES: O=C(CN1C[C@@]2(C[C@H]2F)c2cc(Br)ccc2C1=O)Nc1nc2ccccn2n1] |
| 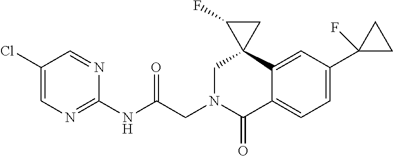 [SMILES: O=C(CN1C[C@]2(C[C@H]2F)c2cc(C3(F)CC3)ccc2C1=O)Nc1ncc(Cl)cn1] | 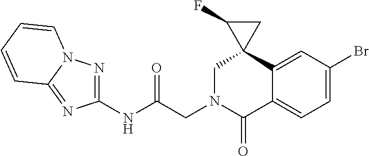 [SMILES: O=C(CN1C[C@]2(C[C@@H]2F)c2cc(Br)ccc2C1=O)Nc1nc2ccccn2n1] |
| 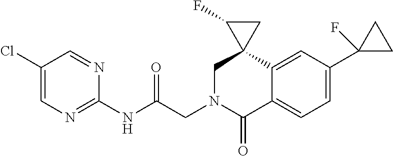 [SMILES: O=C(CN1C[C@@]2(C[C@H]2F)c2cc(C3(F)CC3)ccc2C1=O)Nc1ncc(Cl)cn1] | 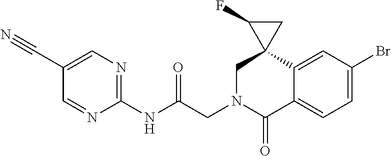 [SMILES: N#Cc1cnc(NC(=O)CN2C[C@@]3(C[C@@H]3F)c3cc(Br)ccc3C2=O)nc1] |
| 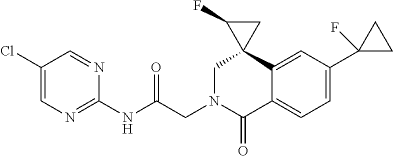 [SMILES: O=C(CN1C[C@]2(C[C@@H]2F)c2cc(C3(F)CC3)ccc2C1=O)Nc1ncc(Cl)cn1] | 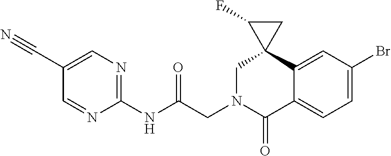 [SMILES: N#Cc1cnc(NC(=O)CN2C[C@]3(C[C@H]3F)c3cc(Br)ccc3C2=O)nc1] |
| 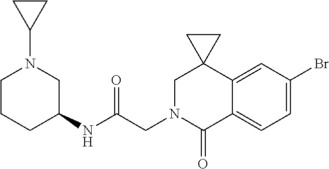 [SMILES: O=C(CN1CC2(CC2)c2cc(Br)ccc2C1=O)N[C@H]1CCCN(C2CC2)C1] | 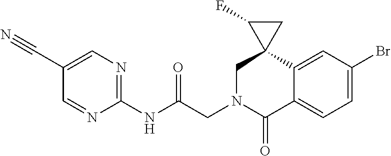 [SMILES: N#Cc1cnc(NC(=O)CN2C[C@@]3(C[C@H]3F)c3cc(Br)ccc3C2=O)nc1] |
| 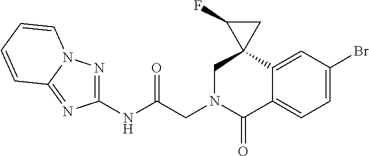 [SMILES: O=C(CN1C[C@@]2(C[C@@H]2F)c2cc(Br)ccc2C1=O)Nc1nc2ccccn2n1] | 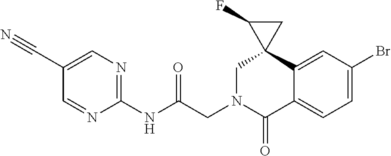 [SMILES: N#Cc1cnc(NC(=O)CN2C[C@]3(C[C@@H]3F)c3cc(Br)ccc3C2=O)nc1] |
| 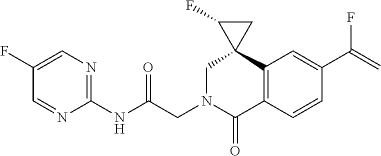 [SMILES: C=C(F)c1ccc2c(c1)[C@@]1(C[C@H]1F)CN(CC(=O)Nc1ncc(F)cn1)C2=O] | 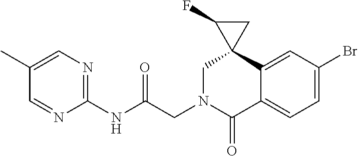 [SMILES: Cc1cnc(NC(=O)CN2C[C@]3(C[C@@H]3F)c3cc(Br)ccc3C2=O)nc1] |
| 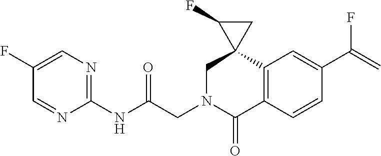 [SMILES: C=C(F)c1ccc2c(c1)[C@]1(C[C@@H]1F)CN(CC(=O)Nc1ncc(F)cn1)C2=O] | 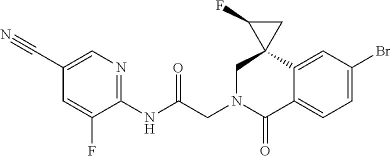 [SMILES: N#Cc1cnc(NC(=O)CN2C[C@@]3(C[C@@H]3F)c3cc(Br)ccc3C2=O)c(F)c1] |
| 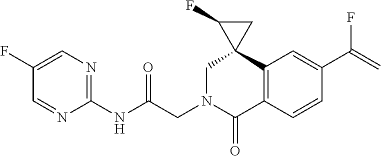 [SMILES: C=C(F)c1ccc2c(c1)[C@@]1(C[C@@H]1F)CN(CC(=O)Nc1ncc(F)cn1)C2=O] | 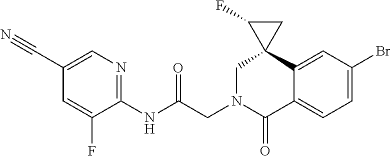 [SMILES: N#Cc1cnc(NC(=O)CN2C[C@]3(C[C@H]3F)c3cc(Br)ccc3C2=O)c(F)c1] |
| 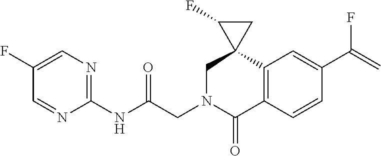 [SMILES: C=C(F)c1ccc2c(c1)[C@]1(C[C@H]1F)CN(CC(=O)Nc1ncc(F)cn1)C2=O] | 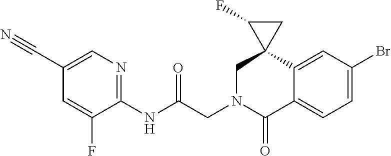 [SMILES: N#Cc1cnc(NC(=O)CN2C[C@@]3(C[C@H]3F)c3cc(Br)ccc3C2=O)c(F)c1] |
| 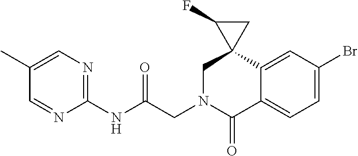 [SMILES: Cc1cnc(NC(=O)CN2C[C@@]3(C[C@@H]3F)c3cc(Br)ccc3C2=O)nc1] | 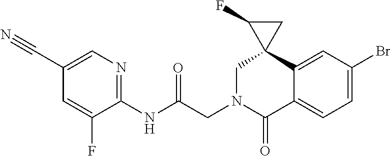 [SMILES: N#Cc1cnc(NC(=O)CN2C[C@]3(C[C@@H]3F)c3cc(Br)ccc3C2=O)c(F)c1] |
| 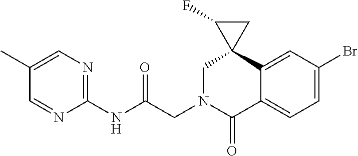 [SMILES: Cc1cnc(NC(=O)CN2C[C@]3(C[C@H]3F)c3cc(Br)ccc3C2=O)nc1] | 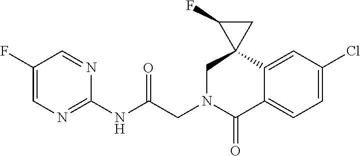 [SMILES: O=C(CN1C[C@@]2(C[C@@H]2F)c2cc(Cl)ccc2C1=O)Nc1ncc(F)cn1] |
| 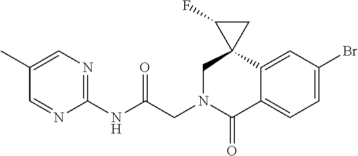 [SMILES: Cc1cnc(NC(=O)CN2C[C@@]3(C[C@H]3F)c3cc(Br)ccc3C2=O)nc1] | 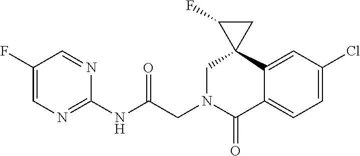 [SMILES: O=C(CN1C[C@]2(C[C@H]2F)c2cc(Cl)ccc2C1=O)Nc1ncc(F)cn1] |
| 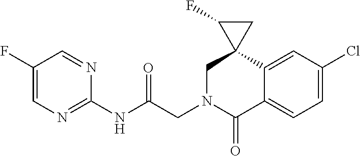 [SMILES: O=C(CN1C[C@@]2(C[C@H]2F)c2cc(Cl)ccc2C1=O)Nc1ncc(F)cn1] | 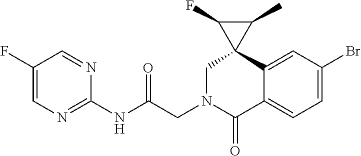 [SMILES: C[C@@H]1[C@H](F)[C@@]12CN(CC(=O)Nc1ncc(F)cn1)C(=O)c1ccc(Br)cc12] |
| 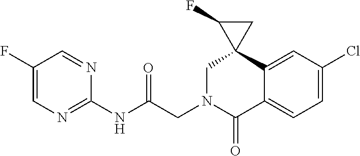 [SMILES: O=C(CN1C[C@]2(C[C@@H]2F)c2cc(Cl)ccc2C1=O)Nc1ncc(F)cn1] | 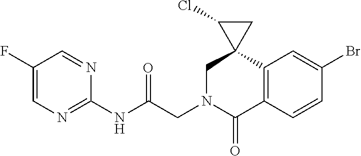 [SMILES: O=C(CN1C[C@@]2(C[C@H]2Cl)c2cc(Br)ccc2C1=O)Nc1ncc(F)cn1] |
| 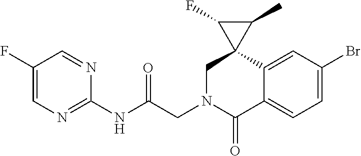 [SMILES: C[C@@H]1[C@@H](F)[C@]12CN(CC(=O)Nc1ncc(F)cn1)C(=O)c1ccc(Br)cc12] | 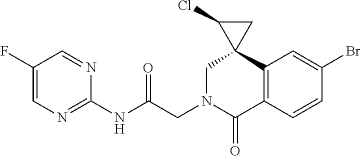 [SMILES: O=C(CN1C[C@]2(C[C@@H]2Cl)c2cc(Br)ccc2C1=O)Nc1ncc(F)cn1] |
| 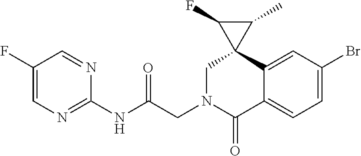 [SMILES: C[C@H]1[C@H](F)[C@@]12CN(CC(=O)Nc1ncc(F)cn1)C(=O)c1ccc(Br)cc12] | 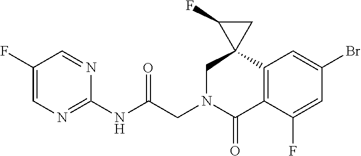 [SMILES: O=C(CN1C[C@@]2(C[C@@H]2F)c2cc(Br)cc(F)c2C1=O)Nc1ncc(F)cn1] |
| 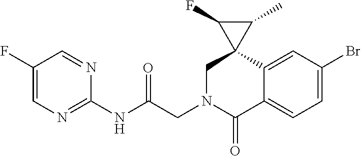 [SMILES: C[C@H]1[C@H](F)[C@]12CN(CC(=O)Nc1ncc(F)cn1)C(=O)c1ccc(Br)cc12] | 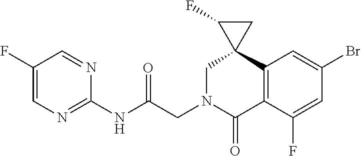 [SMILES: O=C(CN1C[C@]2(C[C@H]2F)c2cc(Br)cc(F)c2C1=O)Nc1ncc(F)cn1] |
| 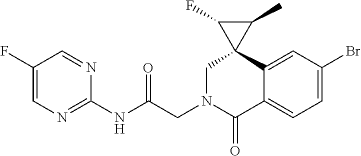 [SMILES: C[C@@H]1[C@@H](F)[C@@]12CN(CC(=O)Nc1ncc(F)cn1)C(=O)c1ccc(Br)cc12] | 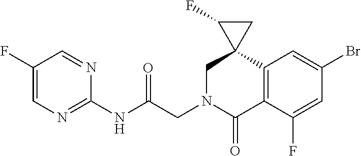 [SMILES: O=C(CN1C[C@@]2(C[C@H]2F)c2cc(Br)cc(F)c2C1=O)Nc1ncc(F)cn1] |
| 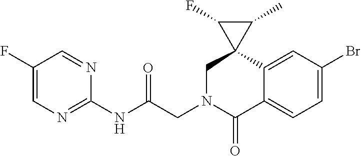 [SMILES: C[C@H]1[C@@H](F)[C@]12CN(CC(=O)Nc1ncc(F)cn1)C(=O)c1ccc(Br)cc12] | 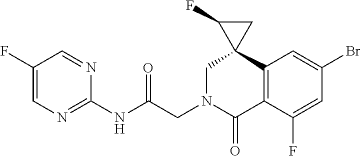 [SMILES: O=C(CN1C[C@]2(C[C@@H]2F)c2cc(Br)cc(F)c2C1=O)Nc1ncc(F)cn1] |
| 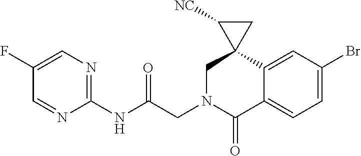 [SMILES: N#C[C@@H]1C[C@@]12CN(CC(=O)Nc1ncc(F)cn1)C(=O)c1ccc(Br)cc12] | 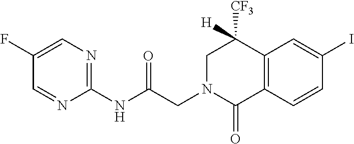 [SMILES: O=C(CN1C[C@H](C(F)(F)F)c2cc(I)ccc2C1=O)Nc1ncc(F)cn1] |
| 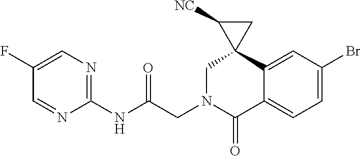 [SMILES: N#C[C@H]1C[C@]12CN(CC(=O)Nc1ncc(F)cn1)C(=O)c1ccc(Br)cc12] | 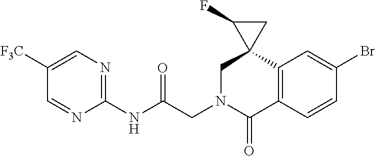 [SMILES: O=C(CN1C[C@@]2(C[C@@H]2F)c2cc(Br)ccc2C1=O)Nc1ncc(C(F)(F)F)cn1] |
| 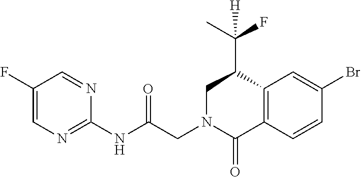 | 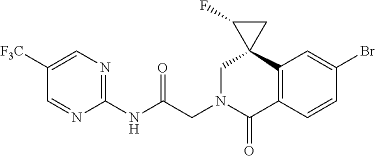 [SMILES: O=C(CN1C[C@]2(C[C@H]2F)c2cc(Br)ccc2C1=O)Nc1ncc(C(F)(F)F)cn1] |
| 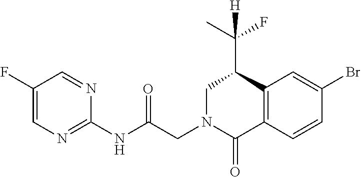 | 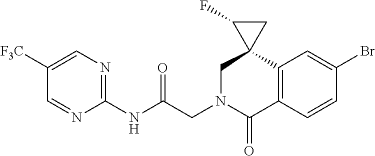 [SMILES: O=C(CN1C[C@@]2(C[C@H]2F)c2cc(Br)ccc2C1=O)Nc1ncc(C(F)(F)F)cn1] |
| 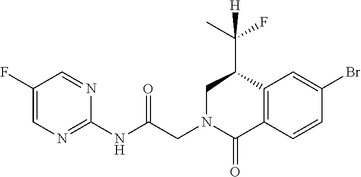 | 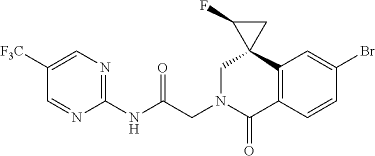 [SMILES: O=C(CN1C[C@]2(C[C@@H]2F)c2cc(Br)ccc2C1=O)Nc1ncc(C(F)(F)F)cn1] |
| 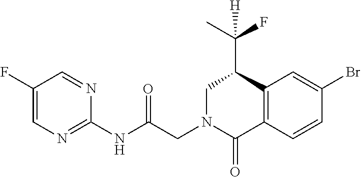 | 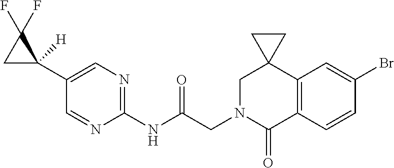 [SMILES: O=C(CN1CC2(CC2)c2cc(Br)ccc2C1=O)Nc1ncc([C@H]2CC2(F)F)cn1] |
| 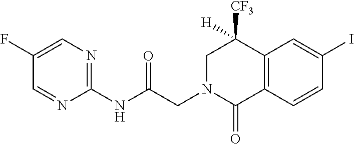 [SMILES: O=C(CN1C[C@@H](C(F)(F)F)c2cc(I)ccc2C1=O)Nc1ncc(F)cn1] | 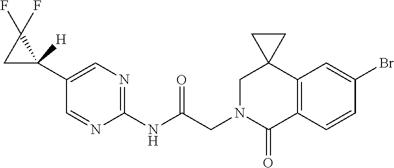 [SMILES: O=C(CN1CC2(CC2)c2cc(Br)ccc2C1=O)Nc1ncc([C@@H]2CC2(F)F)cn1] |
| 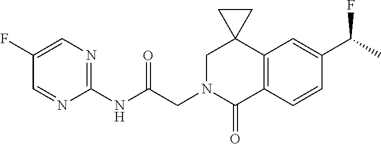 | 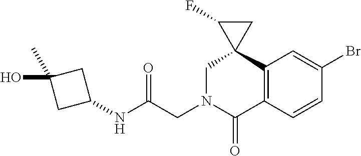 [SMILES: C[C@]1(O)C[C@H](NC(=O)CN2C[C@]3(C[C@H]3F)c3cc(Br)ccc3C2=O)C1] |
| 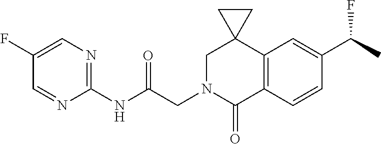 | 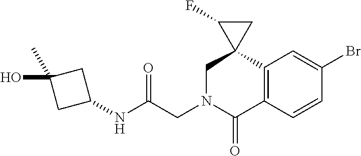 [SMILES: C[C@]1(O)C[C@H](NC(=O)CN2C[C@@]3(C[C@H]3F)c3cc(Br)ccc3C2=O)C1] |
| 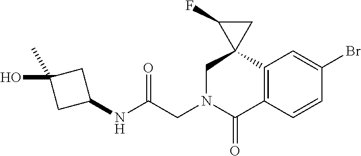 [SMILES: C[C@]1(O)C[C@@H](NC(=O)CN2C[C@@]3(C[C@@H]3F)c3cc(Br)ccc3C2=O)C1] | 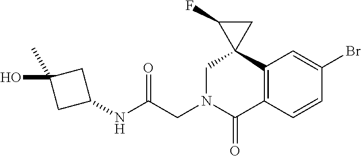 [SMILES: C[C@]1(O)C[C@H](NC(=O)CN2C[C@]3(C[C@@H]3F)c3cc(Br)ccc3C2=O)C1] |
| 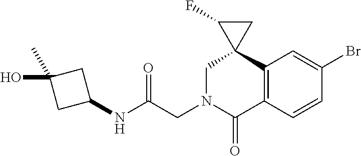 [SMILES: C[C@]1(O)C[C@@H](NC(=O)CN2C[C@]3(C[C@H]3F)c3cc(Br)ccc3C2=O)C1] | 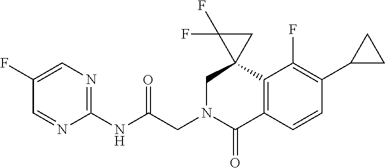 [SMILES: O=C(CN1C[C@@]2(CC2(F)F)c2c(ccc(C3CC3)c2F)C1=O)Nc1ncc(F)cn1] |
| 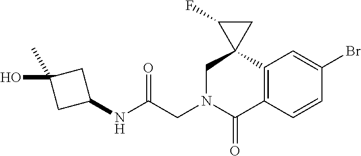 [SMILES: C[C@]1(O)C[C@@H](NC(=O)CN2C[C@@]3(C[C@H]3F)c3cc(Br)ccc3C2=O)C1] | 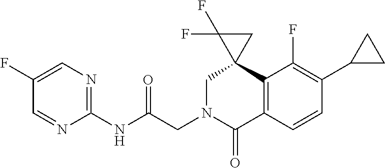 [SMILES: O=C(CN1C[C@]2(CC2(F)F)c2c(ccc(C3CC3)c2F)C1=O)Nc1ncc(F)cn1] |
| 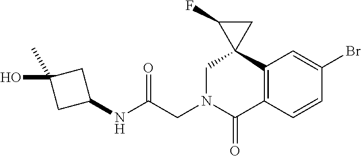 [SMILES: C[C@]1(O)C[C@@H](NC(=O)CN2C[C@]3(C[C@@H]3F)c3cc(Br)ccc3C2=O)C1] | 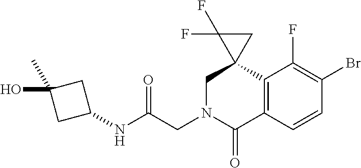 [SMILES: C[C@]1(O)C[C@H](NC(=O)CN2C[C@@]3(CC3(F)F)c3c(ccc(Br)c3F)C2=O)C1] |
| 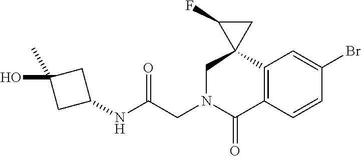 [SMILES: C[C@]1(O)C[C@H](NC(=O)CN2C[C@@]3(C[C@@H]3F)c3cc(Br)ccc3C2=O)C1] | 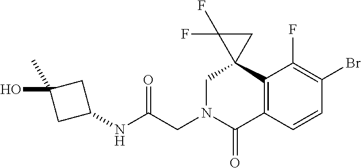 [SMILES: C[C@]1(O)C[C@H](NC(=O)CN2C[C@]3(CC3(F)F)c3c(ccc(Br)c3F)C2=O)C1] |
| 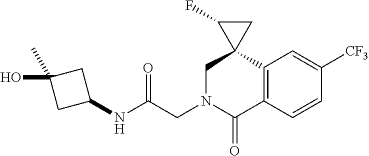 [SMILES: C[C@]1(O)C[C@@H](NC(=O)CN2C[C@@]3(C[C@H]3F)c3cc(C(F)(F)F)ccc3C2=O)C1] | 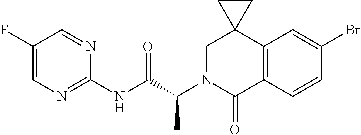 |
| 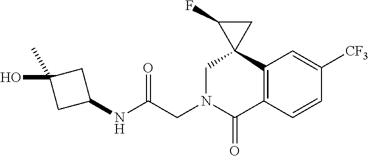 [SMILES: C[C@]1(O)C[C@@H](NC(=O)CN2C[C@]3(C[C@@H]3F)c3cc(C(F)(F)F)ccc3C2=O)C1] | 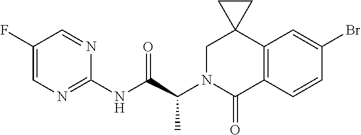 |
| 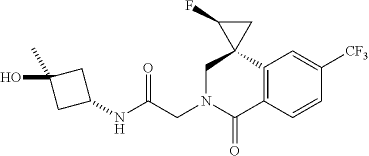 [SMILES: C[C@]1(O)C[C@H](NC(=O)CN2C[C@@]3(C[C@@H]3F)c3cc(C(F)(F)F)ccc3C2=O)C1] | 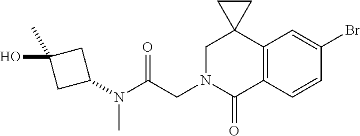 |
| 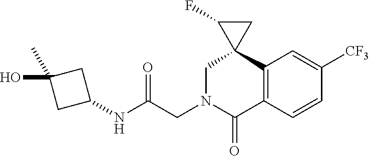 | 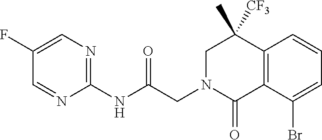 [SMILES: C[C@]1(C(F)(F)F)CN(CC(=O)Nc2ncc(F)cn2)C(=O)c2c(Br)cccc21] |
| 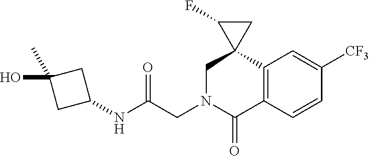 | 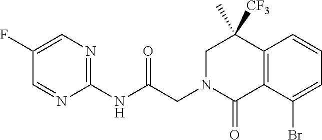 [SMILES: C[C@@]1(C(F)(F)F)CN(CC(=O)Nc2ncc(F)cn2)C(=O)c2c(Br)cccc21] |
| 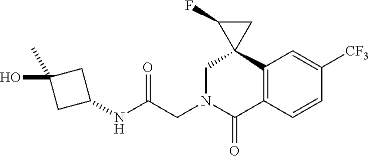 | 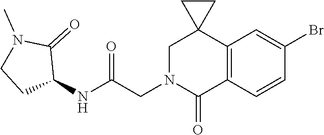 |
| 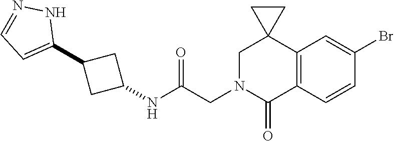 | 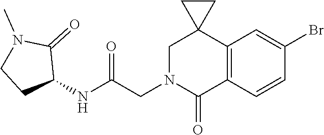 |
| | 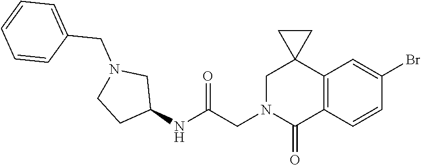 [SMILES: O=C(CN1CC2(CC2)c2cc(Br)ccc2C1=O)N[C@H]1CCN(Cc2ccccc2)C1] |
| 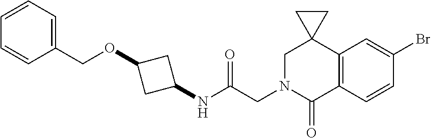 | 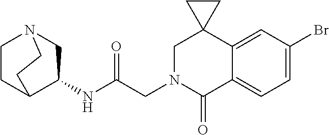 |
| 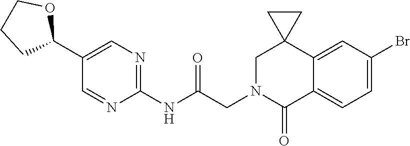 | 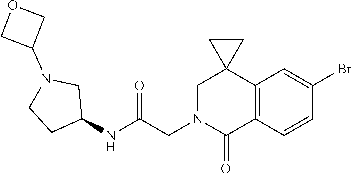 [SMILES: O=C(CN1CC2(CC2)c2cc(Br)ccc2C1=O)N[C@H]1CCN(C2COC2)C1] |
| 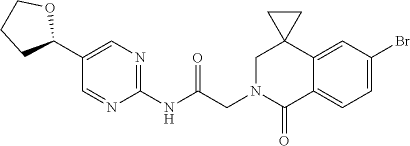 | 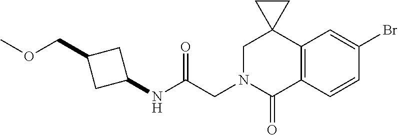 |
| 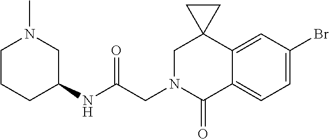 [SMILES: CN1CCC[C@H](NC(=O)CN2CC3(CC3)c3cc(Br)ccc3C2=O)C1] | 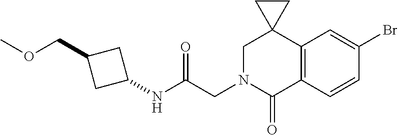 |
| 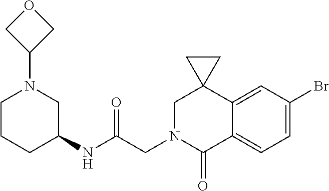 [SMILES: O=C(CN1CC2(CC2)c2cc(Br)ccc2C1=O)N[C@H]1CCCN(C2COC2)C1] | 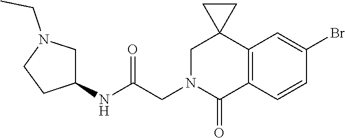 [SMILES: CCN1CC[C@H](NC(=O)CN2CC3(CC3)c3cc(Br)ccc3C2=O)C1] |
| 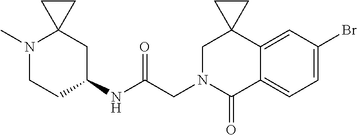 | 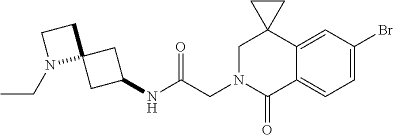 |
| 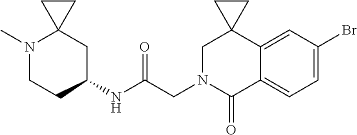 | 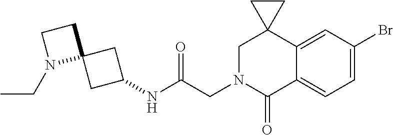 |
| 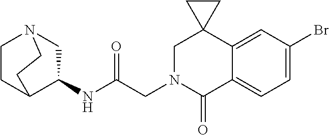 | 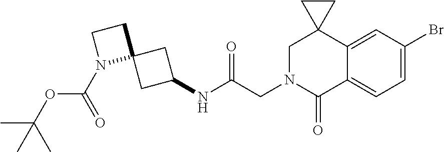 |
| 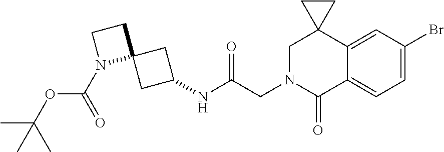 | 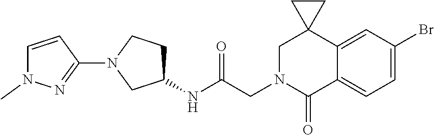 |
| 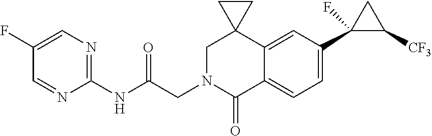 [SMILES: O=C(CN1CC2(CC2)c2cc([C@@]3(F)C[C@H]3C(F)(F)F)ccc2C1=O)Nc1ncc(F)cn1] | 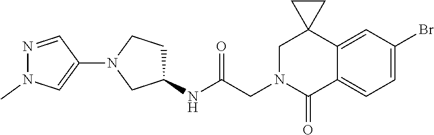 |
| 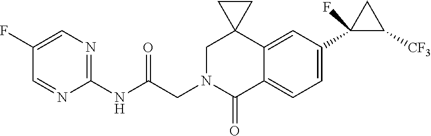 [SMILES: O=C(CN1CC2(CC2)c2cc([C@]3(F)C[C@@H]3C(F)(F)F)ccc2C1=O)Nc1ncc(F)cn1] | 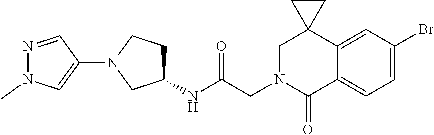 |
| 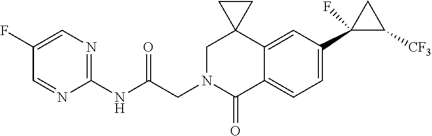 [SMILES: O=C(CN1CC2(CC2)c2cc([C@@]3(F)C[C@@H]3C(F)(F)F)ccc2C1=O)Nc1ncc(F)cn1] | 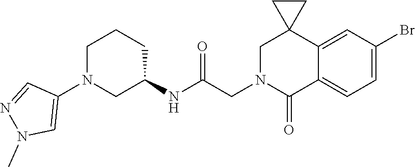 |
| 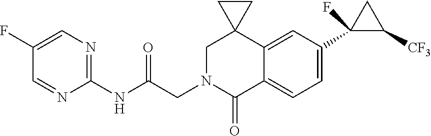 [SMILES: O=C(CN1CC2(CC2)c2cc([C@]3(F)C[C@H]3C(F)(F)F)ccc2C1=O)Nc1ncc(F)cn1] | 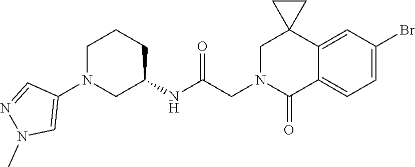 |
| 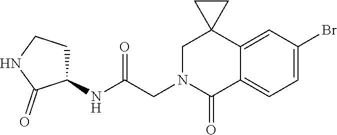 | 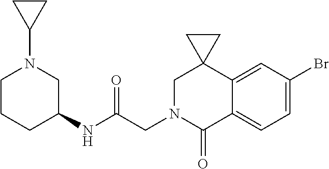 [SMILES: O=C(CN1CC2(CC2)c2cc(Br)ccc2C1=O)N[C@H]1CCCN(C2CC2)C1] |
| 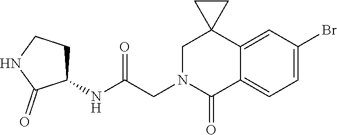 | 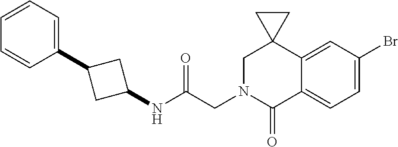 |
| 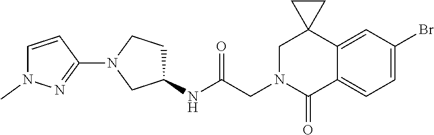 | 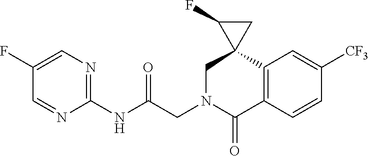 [SMILES: O=C(CN1C[C@@]2(C[C@@H]2F)c2cc(C(F)(F)F)ccc2C1=O)Nc1ncc(F)cn1] |
| 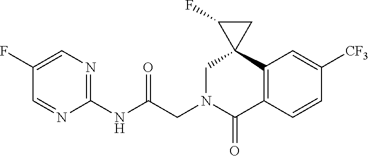 [SMILES: O=C(CN1C[C@]2(C[C@H]2F)c2cc(C(F)(F)F)ccc2C1=O)Nc1ncc(F)cn1] | 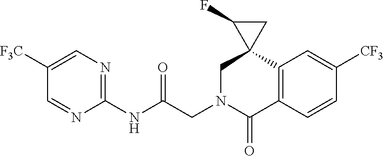 [SMILES: O=C(CN1C[C@@]2(C[C@@H]2F)c2cc(C(F)(F)F)ccc2C1=O)Nc1ncc(C(F)(F)F)cn1] |
| 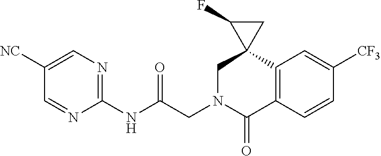 [SMILES: N#Cc1cnc(NC(=O)CN2C[C@@]3(C[C@@H]3F)c3cc(C(F)(F)F)ccc3C2=O)nc1] | 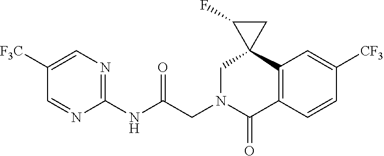 [SMILES: O=C(CN1C[C@]2(C[C@H]2F)c2cc(C(F)(F)F)ccc2C1=O)Nc1ncc(C(F)(F)F)cn1] |
| 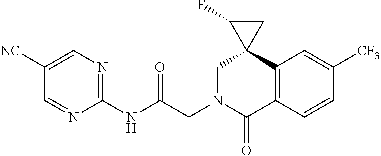 [SMILES: N#Cc1cnc(NC(=O)CN2C[C@]3(C[C@H]3F)c3cc(C(F)(F)F)ccc3C2=O)nc1] | 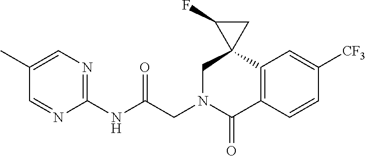 [SMILES: Cc1cnc(NC(=O)CN2C[C@@]3(C[C@@H]3F)c3cc(C(F)(F)F)ccc3C2=O)nc1] |
| 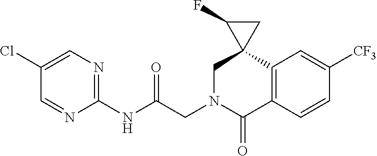 [SMILES: O=C(CN1C[C@@]2(C[C@@H]2F)c2cc(C(F)(F)F)ccc2C1=O)Nc1ncc(Cl)cn1] | 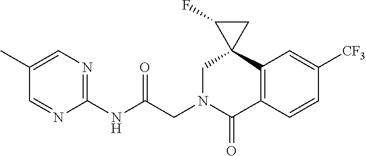 [SMILES: Cc1cnc(NC(=O)CN2C[C@]3(C[C@H]3F)c3cc(C(F)(F)F)ccc3C2=O)nc1] |
| 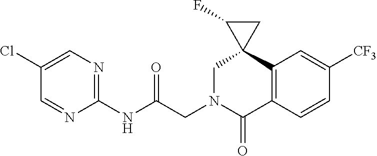 [SMILES: O=C(CN1C[C@]2(C[C@H]2F)c2cc(C(F)(F)F)ccc2C1=O)Nc1ncc(Cl)cn1] | 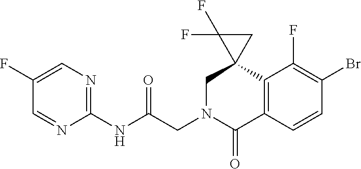 [SMILES: O=C(CN1C[C@@]2(CC2(F)F)c2c(ccc(Br)c2F)C1=O)Nc1ncc(F)cn1] |
| 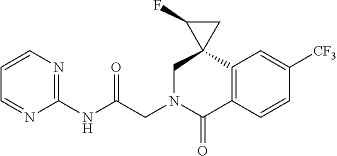 [SMILES: O=C(CN1C[C@@]2(C[C@@H]2F)c2cc(C(F)(F)F)ccc2C1=O)Nc1ncccn1] | 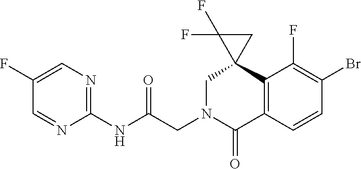 [SMILES: O=C(CN1C[C@]2(CC2(F)F)c2c(ccc(Br)c2F)C1=O)Nc1ncc(F)cn1] |
| 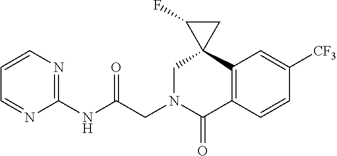 [SMILES: O=C(CN1C[C@]2(C[C@H]2F)c2cc(C(F)(F)F)ccc2C1=O)Nc1ncccn1] | 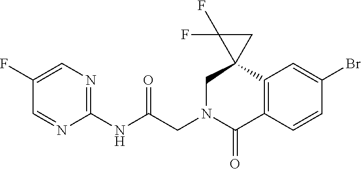 [SMILES: O=C(CN1C[C@@]2(CC2(F)F)c2cc(Br)ccc2C1=O)Nc1ncc(F)cn1] |
| 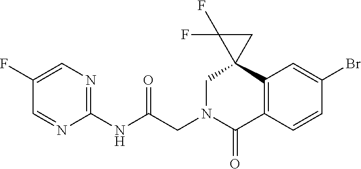 [SMILES: O=C(CN1C[C@]2(CC2(F)F)c2cc(Br)ccc2C1=O)Nc1ncc(F)cn1] | 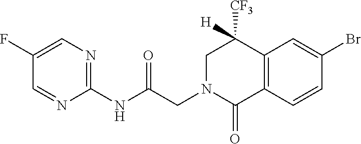 [SMILES: O=C(CN1C[C@H](C(F)(F)F)c2cc(Br)ccc2C1=O)Nc1ncc(F)cn1] |
| 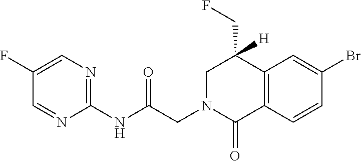 [SMILES: O=C(CN1C[C@H](CF)c2cc(Br)ccc2C1=O)Nc1ncc(F)cn1] | 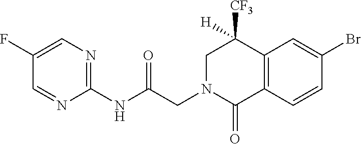 [SMILES: O=C(CN1C[C@@H](C(F)(F)F)c2cc(Br)ccc2C1=O)Nc1ncc(F)cn1] |
| 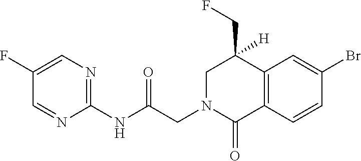 [SMILES: O=C(CN1C[C@@H](CF)c2cc(Br)ccc2C1=O)Nc1ncc(F)cn1] | 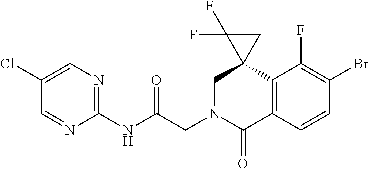 [SMILES: O=C(CN1C[C@@]2(CC2(F)F)c2c(ccc(Br)c2F)C1=O)Nc1ncc(Cl)cn1] |
| 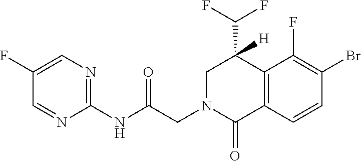 [SMILES: O=C(CN1C[C@H](C(F)F)c2c(ccc(Br)c2F)C1=O)Nc1ncc(F)cn1] | 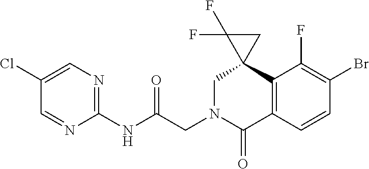 [SMILES: O=C(CN1C[C@]2(CC2(F)F)c2c(ccc(Br)c2F)C1=O)Nc1ncc(Cl)cn1] |
| 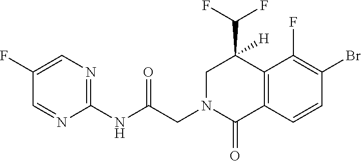 [SMILES: O=C(CN1C[C@@H](C(F)F)c2c(ccc(Br)c2F)C1=O)Nc1ncc(F)cn1] | 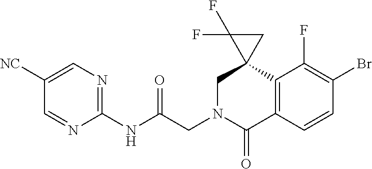 [SMILES: N#Cc1cnc(NC(=O)CN2C[C@@]3(CC3(F)F)c3c(ccc(Br)c3F)C2=O)nc1] |
| 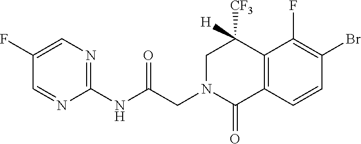 [SMILES: O=C(CN1C[C@H](C(F)(F)F)c2c(ccc(Br)c2F)C1=O)Nc1ncc(F)cn1] | 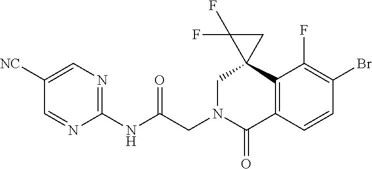 [SMILES: N#Cc1cnc(NC(=O)CN2C[C@]3(CC3(F)F)c3c(ccc(Br)c3F)C2=O)nc1] |
| 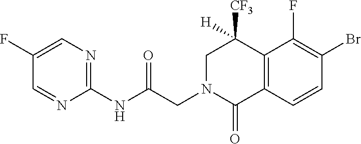 [SMILES: O=C(CN1C[C@@H](C(F)(F)F)c2c(ccc(Br)c2F)C1=O)Nc1ncc(F)cn1] | 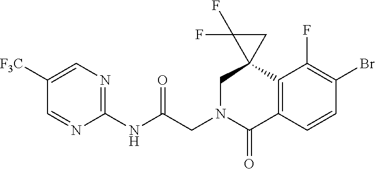 [SMILES: O=C(CN1C[C@@]2(CC2(F)F)c2c(ccc(Br)c2F)C1=O)Nc1ncc(C(F)(F)F)cn1] |
| 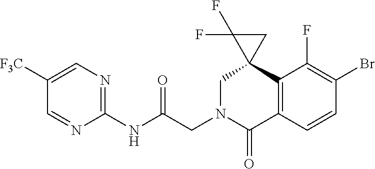 [SMILES: O=C(CN1C[C@]2(CC2(F)F)c2c(ccc(Br)c2F)C1=O)Nc1ncc(C(F)(F)F)cn1] | 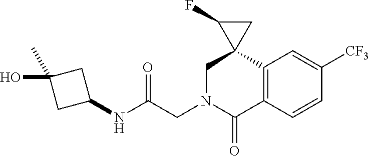 |
| 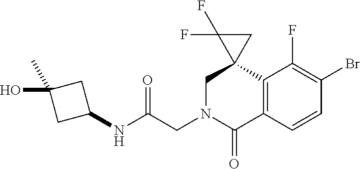 | 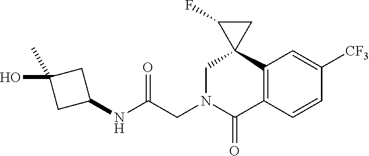 |
| 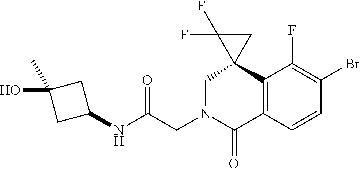 | |

### 3. Methods

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy, and/or veterinary applications. In some embodiments, the subject is a mammal. In certain embodiments, the subject is a human.

The term “therapeutically effective amount” or “effective amount” of a compound described herein or a pharmaceutically acceptable salt, isotopically enriched analog, stereoisomer, mixture of stereoisomers, or prodrug thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition of as described herein. The therapeutically effective amount may vary depending on the subject, and disease or condition being treated, the weight and age of the subject, the severity of the disease or condition, and the manner of administering, which can readily be determined by one of ordinary skill in the art.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The compounds may be further characterized to examine the safety or tolerance dosage in human or non-human subjects. Such properties may be examined using commonly known methods to those skilled in the art.

In certain embodiments, provided are compounds, or a pharmaceutically acceptable salt, isotopically enriched analog, stereoisomer, mixture of stereoisomers, or prodrug thereof, that modulate the activity of NLR Family Pyrin Domain Containing 3 (NLRP3). In certain embodiments, the compounds provided herein, or a pharmaceutically acceptable salt, isotopically enriched analog, stereoisomer, mixture of stereoisomers, or prodrug thereof, inhibit the activation of NLRP3.

NLR proteins are involved in the immune system, helping to start and regulate the immune system's response to injury, toxins, or invasion by microorganisms. NLRP3 (also known as cryopyrin, NALP3, LRR and PYD domains-containing protein 3), is a protein encoded by the NLRP3 gene (also known as CIAS1). Once activated, NLRP3 molecules assemble, along with other proteins, into inflammasomes. The activation of NLRP3 by cellular stress leads to inflammasome activation and downstream proteolytic events, including the formation of active proinflammatory cytokines such as interleukin (IL)-1β and IL-18 which are then secreted. Among other cytokines, IL-1β and IL-18 are known mediators of inflammation, e.g., artery wall inflammation, atherosclerosis and the aging process.

In certain embodiments, provided is a method of inhibiting inflammasome (e.g., the NLRP3 inflammasome) activity comprising contacting a cell with an effective amount of a compound disclosed herein, or a pharmaceutically acceptable salt, isotopically enriched analog, stereoisomer, mixture of stereoisomers, or prodrug thereof. The inhibiting can be in vitro or in vivo.

In certain embodiments, provided is a compound as disclosed herein, or a pharmaceutically acceptable salt, isotopically enriched analog, stereoisomer, mixture of stereoisomers, or prodrug thereof, for use in inhibiting inflammasome (e.g., the NLRP3 inflammasome) activity (e.g., in vitro or in vivo).

In certain embodiments, the present disclosure provides use of a compound as disclosed herein, or a pharmaceutically acceptable salt, isotopically enriched analog, stereoisomer, mixture of stereoisomers, or prodrug thereof, in the manufacture of a medicament for inhibiting inflammasome (e.g., the NLRP3 inflammasome) activity (e.g., in vitro or in vivo).

Chronic inflammation responses have been associated with various types of cancer. During malignant transformation or cancer therapy, inflammasomes may become activated in response to certain signals; and IL-Iβ expression is elevated in a variety of cancers (e.g., breast, prostate, colon, lung, head and neck cancers, melanomas, etc.), where patients with IL-IP producing tumors generally have a worse prognosis.

In certain embodiments, provided is a method for treating a disease or condition mediated, at least in part, by NLRP3, comprising administering an effective amount of a compound disclosed herein, or a pharmaceutically acceptable salt, isotopically enriched analog, stereoisomer, mixture of stereoisomers, or prodrug thereof, to a subject in need thereof.

In certain embodiments, provided is a method for treating a disease or condition selected from an autoinflammatory disorder, an autoimmune disorder, a neurodegenerative disease or cancer, comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, provided is a compound as disclosed herein, or a pharmaceutically acceptable salt, isotopically enriched analog, stereoisomer, mixture of stereoisomers, or prodrug thereof, for use in treating an autoinflammatory disorder, an autoimmune disorder, a neurodegenerative disease or cancer in a subject in need thereof.

In certain embodiments, the present disclosure provides use of a compound as disclosed herein, or a pharmaceutically acceptable salt, isotopically enriched analog, stereoisomer, mixture of stereoisomers, or prodrug thereof in the manufacture of a medicament for treating or preventing an autoinflammatory disorder, an autoimmune disorder, a neurodegenerative disease or cancer in a subject in need thereof.

In certain embodiments, provided is a method for treating inflammation, an auto-immune disease, cancer, an infection, a central nervous system disease, a metabolic disease, a cardiovascular disease, a respiratory disease, a liver disease, a renal disease, an ocular disease, a skin disease, a lymphatic condition, a psychological disorder, graft versus host disease, allodynia, and any disease where an individual has been determined to carry a germline or somatic non-silent mutation in NLRP3, comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, the disease or condition may be a disease or condition of the immune system, the cardiovascular system, the endocrine system, the gastrointestinal tract, the renal system, the hepatic system, the metabolic system, the respiratory system, the central nervous system, may be a cancer or other malignancy, and/or may be caused by or associated with a pathogen. It will be appreciated that these general embodiments defined according to broad categories of diseases, disorders and conditions are not mutually exclusive.

In certain embodiments, the disease or condition includes, inflammation, including inflammation occurring as a result of an inflammatory disorder, e.g. an autoinflammatory disease, inflammation occurring as a symptom of a non-inflammatory disorder, inflammation occurring as a result of infection, or inflammation secondary to trauma, injury or autoimmunity; auto-immune diseases such as acute disseminated encephalitis, Addison's disease, ankylosing spondylitis, antiphospholipid antibody syndrome (APS), anti-synthetase syndrome, aplastic anemia, autoimmune adrenalitis, autoimmune hepatitis, autoimmune oophoritis, autoimmune polyglandular failure, autoimmune thyroiditis, Coeliac disease, Crohn's disease, type 1 diabetes (T1D), Goodpasture's syndrome, Graves' disease, Guillain-Barré syndrome (GBS), Hashimoto's disease, idiopathic thrombocytopenic purpura, Kawasaki's disease, lupus erythematosus including systemic lupus erythematosus (SLE), multiple sclerosis (MS) including primary progressive multiple sclerosis (PPMS), secondary progressive multiple sclerosis (SPMS) and relapsing remitting multiple sclerosis (RRMS), myasthenia gravis, opsoclonus myoclonus syndrome (OMS), optic neuritis, Ord's thyroiditis, pemphigus, pernicious anaemia, polyarthritis, primary biliary cirrhosis, rheumatoid arthritis (RA), psoriatic arthritis, juvenile idiopathic arthritis or Still's disease, refractory gouty arthritis, Reiter's syndrome, Sjögren's syndrome, systemic sclerosis a systemic connective tissue disorder, Takayasu's arteritis, temporal arteritis, warm autoimmune hemolytic anemia, Wegener's granulomatosis, alopecia universalis, Behçet's disease, Chagas' disease, dysautonomia, endometriosis, hidradenitis suppurativa (HS), interstitial cystitis, neuromyotonia, psoriasis, sarcoidosis, scleroderma, ulcerative colitis, Schnitzler syndrome, macrophage activation syndrome, Blau syndrome, vitiligo or vulvodynia; cancer including lung cancer, pancreatic cancer, gastric cancer, myelodysplastic syndrome, leukemia including acute lymphocytic leukaemia (ALL) and acute myeloid leukaemia (AML), adrenal cancer, anal cancer, basal and squamous cell skin cancer, bile duct cancer, bladder cancer, bone cancer, brain and spinal cord tumors, breast cancer, cervical cancer, chronic lymphocytic leukaemia (CLL), chronic myeloid leukaemia (CML), chronic myelomonocytic leukaemia (CMML), colorectal cancer, endometrial cancer, oesophagus cancer, Ewing family of tumors, eye cancer, gallbladder cancer, gastrointestinal carcinoid tumours, gastrointestinal stromal tumor (GIST), gestational trophoblastic disease, glioma, Hodgkin lymphoma, Kaposi sarcoma, kidney cancer, laryngeal and hypopharyngeal cancer, liver cancer, lung carcinoid tumor, lymphoma including cutaneous T cell lymphoma, malignant mesothelioma, melanoma skin cancer, Merkel cell skin cancer, multiple myeloma, nasal cavity and paranasal sinuses cancer, nasopharyngeal cancer, neuroblastoma, non-Hodgkin lymphoma, non-small cell lung cancer, oral cavity and oropharyngeal cancer, osteosarcoma, ovarian cancer, penile cancer, pituitary tumours, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer including anaplastic thyroid cancer, uterine sarcoma, vaginal cancer, vulvar cancer, Waldenstrom macroglobulinemia, and Wilms tumor; infections including viral infections (e.g. from influenza virus, human immunodeficiency virus (HIV), alphavirus (such as Chikungunya and Ross River virus), flaviviruses (such as Dengue virus and Zika virus), herpes viruses (such as Epstein Barr Virus, cytomegalovirus, Varicella-zoster virus, and KSHV), poxviruses (such as vaccinia virus (Modified vaccinia virus Ankara) and Myxoma virus), adenoviruses (such as Adenovirus 5), or papillomavirus), bacterial infections (e.g. from Staphylococcus aureus, Helicobacter pylori, Bacillus anthracis, Bordatella pertussis, Burkholderia pseudomallei, Corynebacterium diptheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi or Yersinia pestis), fungal infections (e.g. from Candida or Aspergillus species), protozoan infections (e.g. from Plasmodium, Babesia, Giardia, Entamoeba, Leishmania or Trypanosomes), helminth infections (e.g. from schistosoma, roundworms, tapeworms or flukes) and prion infections; central nervous system diseases such as Parkinson's disease, Alzheimer's disease, dementia, motor neuron disease, Huntington's disease, cerebral malaria, brain injury from pneumococcal meningitis, intracranial aneurysms, traumatic brain injury, and amyotrophic lateral sclerosis; metabolic diseases such as type 2 diabetes (T2D), atherosclerosis, obesity, gout, and pseudo-gout; cardiovascular diseases such as hypertension, ischaemia, reperfusion injury including post-MI ischemic reperfusion injury, stroke including ischemic stroke, transient ischemic attack, myocardial infarction including recurrent myocardial infarction, heart failure including congestive heart failure and heart failure with preserved ejection fraction, embolism, aneurysms including abdominal aortic aneurysm, and pericarditis including Dressler's syndrome; respiratory diseases including chronic obstructive pulmonary disorder (COPD), asthma such as allergic asthma and steroid-resistant asthma, asbestosis, silicosis, nanoparticle induced inflammation, cystic fibrosis and idiopathic pulmonary fibrosis; liver diseases including non-alcoholic fatty liver disease (NAFLD) and non-alcoholic steatohepatitis (NASH) including advanced fibrosis stages F3 and F4; alcoholic fatty liver disease (AFLD), and alcoholic steatohepatitis (ASH); renal diseases including chronic kidney disease, oxalate nephropathy, nephrocalcinosis, glomerulonephritis, and diabetic nephropathy; ocular diseases including those of the ocular epithelium, age-related macular degeneration (AMD) (dry and wet), uveitis, corneal infection, diabetic retinopathy, optic nerve damage, dry eye, and glaucoma; skin diseases including dermatitis such as contact dermatitis and atopic dermatitis, contact hypersensitivity, sunburn, skin lesions, hidradenitis suppurativa (HS), other cyst-causing skin diseases, and acne conglobata; lymphatic conditions such as lymphangitis and Castleman's disease; psychological disorders such as depression and psychological stress; graft versus host disease; allodynia including mechanical allodynia; and any disease where an individual has been determined to carry a germline or somatic non-silent mutation in NLRP3.

In certain embodiments, the disease, disorder or condition is an autoinflammatory disease such as cryopyrin-associated periodic syndromes (CAPS), Muckle-Wells syndrome (MWS), familial cold autoinflammatory syndrome (FCAS), familial Mediterranean fever (FMF), neonatal onset multisystem inflammatory disease (NOMID), tumor Necrosis Factor (TNF) Receptor-Associated Periodic Syndrome (TRAPS), hyperimmunoglobulinemia D and periodic fever syndrome (HIDS), deficiency of interleukin 1 receptor antagonist (DIRA), Majeed syndrome, pyogenic arthritis, pyoderma gangrenosum and acne syndrome (PAPA), adult-onset Still's disease (AOSD), haploinsufficiency of A20 (HA20), pediatric granulomatous arthritis (PGA), PLCG2-associated antibody deficiency and immune dysregulation (PLAID), PLCG2-associated autoinflammatory, antibody deficiency and immune dysregulation (APLAID), or sideroblastic anaemia with B-cell immunodeficiency, periodic fevers and developmental delay (SIFD).

In certain embodiments, provided is a method for treating a disease or condition selected from an autoinflammatory disorder and/or an autoimmune disorder selected from cryopyrin-associated autoinflammatory syndrome (CAPS; e.g., familial cold autoinflammatory syndrome (FCAS)), Muckle-Wells syndrome (MWS), chronic infantile neurological cutaneous and articular (CINCA) syndrome, neonatal-onset multisystem inflammatory disease (NOMID), familial Mediterranean fever and nonalcoholic fatty liver disease (NAFLD), non-alcoholic steatohepatitis (NASH), gout, rheumatoid arthritis, osteoarthritis, Crohn's disease, chronic obstructive pulmonary disease (COPD), chronic kidney disease (CKD), fibrosis, obesity, type 2 diabetes, and multiple sclerosis and neuroinflammation occurring in protein misfolding diseases (e.g., Prion diseases) comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, provided is a method for treating a disease or condition selected from cryopyrin-associated periodic syndromes (CAPS), Muckle-Wells syndrome (MWS), familial cold autoinflammatory syndrome (FCAS), neonatal onset multisystem inflammatory disease (NOMID), familial Mediterranean fever (FMF), pyogenic arthritis, pyoderma gangrenosum and acne syndrome (PAPA); hyperimmunoglobulinemia D and periodic fever syndrome (HIDS), Tumour Necrosis Factor (TNF), Receptor-Associated Periodic Syndrome (TRAPS), systemic juvenile idiopathic arthritis, adult-onset Still's disease (AOSD), relapsing polychondritis, Schnitzler's syndrome, Sweet's syndrome, Behcet's disease, anti-synthetase syndrome, deficiency of interleukin 1 receptor antagonist (DIRA), and haploinsufficiency of A20 (HA20) comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, provided is a method for treating a disease or condition selected from Alzheimer's disease, atherosclerosis, asthma, allergic airway inflammation, cryopyrin-associated periodic syndromes, gout, inflammatory bowel disease and related disorders, nonalcoholic fatty liver disease (NAFLD), nonalcoholic steatohepatitis (NASH), hypertension, myocardial infarction, multiple sclerosis, experimental autoimmune encephalitis, oxalate-induced nephropathy, hyperinflammation following influenza infection, graft-versus-host disease, stroke, silicosis, type 1 diabetes, obesity-induced inflammation or insulin resistance, rheumatoid arthritis, myelodysplastic syndrome, contact hypersensitivity, joint inflammation triggered by chikungunya virus, or traumatic brain injury comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, provided is a method for treating a disease or condition that is mediated, at least in part, by TNF-α. In certain embodiments, the disease or condition is resistant to treatment with an anti-TNF-α agent. In some embodiments, the disease is a gut disease or condition. In some embodiments the disease or condition is inflammatory bowel disease, Crohn's disease, or ulcerative colitis. In some embodiments, a compound disclosed herein or a pharmaceutically acceptable salt, isotopically enriched analog, stereoisomer, mixture of stereoisomers, or prodrug thereof is adminstered in combination with an anti-TNF-α agent. In some embodiments, the anti-TNF-α agent is Infliximab, Etanercept, Certolizumab pegol, Golimumab, or Adalimumab.

In certain embodiments, the disease or condition is an autoinflammatory disorder, an autoimmune disorder, a neurodegenerative disease, or cancer.

In certain embodiments, the disease or condition is an autoinflammatory disorder and/or an autoimmune disorder.

In certain embodiments, the disease or condition is a neurodegenerative disease.

In certain embodiments, the disease or condition is Parkinson's disease or Alzheimer's disease.

In certain embodiments, provided is a method for treating cancer, comprising administering an effective amount of a compound disclosed herein, or a pharmaceutically acceptable salt, isotopically enriched analog, stereoisomer, mixture of stereoisomers, or prodrug thereof, to a subject in need thereof.

In certain embodiments, the cancer is metastasizing cancer, gastrointestinal cancer, skin cancer, non-small-cell lung carcinoma, or colorectal adenocarcinoma.

In certain embodiments, provided is a compound as disclosed herein, or a pharmaceutically acceptable salt, isotopically enriched analog, stereoisomer, mixture of stereoisomers, or prodrug thereof for use in treating a neurodegenerative disease (e.g., Parkinson's disease or Alzheimer's disease) in a subject in need thereof.

In certain embodiments, provided is a compound as disclosed herein, or a pharmaceutically acceptable salt, isotopically enriched analog, stereoisomer, mixture of stereoisomers, or prodrug thereof, for use in treating cancer in a subject in need thereof.

In certain embodiments, a compound as disclosed herein, or a pharmaceutically acceptable salt, isotopically enriched analog, stereoisomer, mixture of stereoisomers, or prodrug thereof, may be administered alone as a sole therapy or can be administered in addition with one or more other substances and/or treatments. Such conjoint treatment may be achieved by way of the simultaneous, sequential or separate administration of the individual components of the treatment.

For example, therapeutic effectiveness may be enhanced by administration of an adjuvant (i.e., by itself the adjuvant may only have minimal therapeutic benefit, but in combination with another therapeutic agent, the overall therapeutic benefit to the individual is enhanced). Alternatively, by way of example only, the benefit experienced by an individual may be increased by administering compound as disclosed herein, or a pharmaceutically acceptable salt, isotopically enriched analog, stereoisomer, mixture of stereoisomers, or prodrug thereof, with another therapeutic agent (which also includes a therapeutic regimen) that also has therapeutic benefit.

Other embodiments include use of the presently disclosed compounds in therapy.

### 4. Kits

Provided herein are also kits that include a compound of the disclosure, or a pharmaceutically acceptable salt, isotopically enriched analog, stereoisomer, mixture of stereoisomers, or prodrug thereof, and suitable packaging. In certain embodiments, a kit further includes instructions for use. In one aspect, a kit includes a compound of the disclosure, or a pharmaceutically acceptable salt, isotopically enriched analog, stereoisomer, mixture of stereoisomers, or prodrug thereof, and a label and/or instructions for use of the compounds in the treatment of the indications, including the diseases or conditions, described herein.

Provided herein are also articles of manufacture that include a compound described herein or a pharmaceutically acceptable salt, isotopically enriched analog, stereoisomer, mixture of stereoisomers, or prodrug thereof in a suitable container. The container may be a vial, jar, ampoule, preloaded syringe, or intravenous bag.

### 5. Pharmaceutical Compositions and Modes of Administration

Compounds provided herein are usually administered in the form of pharmaceutical compositions. Thus, provided herein are also pharmaceutical compositions that contain one or more of the compounds described herein, or a pharmaceutically acceptable salt, stereoisomer, mixture of stereoisomers, or prodrug thereof, and one or more pharmaceutically acceptable vehicles selected from carriers, adjuvants, and excipients. Suitable pharmaceutically acceptable vehicles may include, for example, inert solid diluents and fillers, diluents, including sterile aqueous solution and various organic solvents, permeation enhancers, solubilizers, and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal, and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, isotopically enriched analog, stereoisomer, mixture of stereoisomers, or prodrug thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include, e.g., lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, isotopically enriched analog, stereoisomer, mixture of stereoisomers, or prodrug thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, isotopically enriched analog, stereoisomer, mixture of stereoisomers, or prodrug thereof. When referring to these preformulation compositions as homogeneous, the active ingredient may be dispersed evenly throughout the composition so that the composition may be readily subdivided into equally effective unit dosage forms such as tablets, pills, and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

### 6. Dosing

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. In some embodiments, a dosage of from about 0.0001 to about 100 mg per kg of body weight per day, from about 0.001 to about 50 mg of compound per kg of body weight, or from about 0.01 to about 10 mg of compound per kg of body weight may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

### 7. Synthesis of the Compounds

The compounds may be prepared using the methods disclosed herein and routine modifications thereof, which will be apparent given the disclosure herein and methods well known in the art. Conventional and well-known synthetic methods may be used in addition to the teachings herein. The synthesis of typical compounds described herein may be accomplished as described in the following examples. If available, reagents and starting materials may be purchased commercially, e.g., from Sigma Aldrich or other chemical suppliers.

It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, conventional protecting groups (“PG”) may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006). Greene's protective groups in organic synthesis. Hoboken, N.J., Wiley-Interscience, and references cited therein. For example, protecting groups for alcohols, such as hydroxy, include silyl ethers (including trimethylsilyl (TMS), tert-butyldimethylsilyl (TBDMS), tri-iso-propylsilyloxymethyl (TOM), and triisopropylsilyl (TIPS) ethers), which can be removed by acid or fluoride ion, such as NaF, TBAF (tetra-n-butylammonium fluoride), HF-Py, or HF-NEt₃. Other protecting groups for alcohols include acetyl, removed by acid or base, benzoyl, removed by acid or base, benzyl, removed by hydrogenation, methoxyethoxymethyl ether, removed by acid, dimethoxytrityl, removed by acid, methoxymethyl ether, removed by acid, tetrahydropyranyl or tetrahydrofuranyl, removed by acid, and trityl, removed by acid. Examples of protecting groups for amines include carbobenzyloxy, removed by hydrogenolysis p-methoxybenzyl carbonyl, removed by hydrogenolysis, tert-butyloxycarbonyl, removed by concentrated strong acid (such as HCl or CF₃COOH), or by heating to greater than about 80° C., 9-fluorenylmethyloxycarbonyl, removed by base, such as piperidine, acetyl, removed by treatment with a base, benzoyl, removed by treatment with a base, benzyl, removed by hydrogenolysis, carbamate group, removed by acid and mild heating, p-methoxybenzyl, removed by hydrogenolysis, 3,4-dimethoxybenzyl, removed by hydrogenolysis, p-methoxyphenyl, removed by ammonium cerium(IV) nitrate, tosyl, removed by concentrated acid (such as HBr or H₂SO₄) and strong reducing agents (sodium in liquid ammonia or sodium naphthalenide), troc (trichloroethyl chloroformate), removed by Zn insertion in the presence of acetic acid, and sulfonamides (Nosyl & Nps), removed by samarium iodide or tributyltin hydride.

Furthermore, the compounds of this disclosure may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures or obvious modifications thereof, described in standard reference texts such as Fieser and Fieser's Reagents for Organic Synthesis, Volumes 1-15 (John Wiley, and Sons, 1991). Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

### General Synthesis

Scheme I illustrates a general methods which can be employed for the synthesis of compounds described herein, where each of A¹, A², A³, A⁴, R², R³, R⁴, R⁵, R⁶, R⁷, R⁹, and R¹⁰ are each independently as defined herein, each R^{z} is independently hydrogen or C₁₋₆ alkyl, and LG is a leaving group (e.g., halo). It should be understood that derivatization of any one or more of compounds I-1 and I-5, or any product obtained by the process outlined in Scheme I, can be performed to provide various compounds of Formula I.

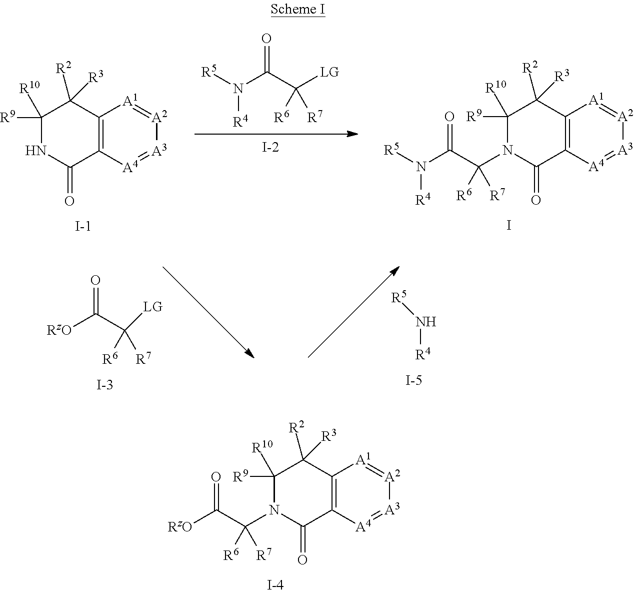

In Scheme I, compounds of formula I can be prepared from compound I-1 by coupling with compound I-2. Alternatively, coupling of compound I-1 with compound I-3 provides compound I-4. An appropriately substituted amine I-5 can be coupled directly with compound I-4 under amide bond forming reaction conditions to yield compounds of formula I. Alternatively, when R^{z} is C₁₋₆ alkyl, the ester can be cleaved to yield the corresponding carboxylic acid derivative, which upon reaction with an appropriately substituted amine I-5 under amide bond forming reaction conditions, yields compounds of formula I.

Appropriate starting materials and reagents can be purchased or prepared by methods known to one of skill in the art. Upon each reaction completion, each of the intermediate or final compounds can be recovered, and optionally purified, by conventional techniques such as neutralization, extraction, precipitation, chromatography, filtration and the like.

In some embodiments, the various substituents of compounds I-1, I-2, I-3, I-4, and I-5 as used in Scheme I are as defined for Formula I. However, derivatization of compounds I-1, I-2, I-3, I-4, and I-5 provides various compounds of Formula I.

In certain embodiments, provided is a process for preparing a compound of Formula I, comprising:
contacting a compound of Formula I-1 with a compound of Formula I-2, under conditions suitable to provide a compound of Formula I.

In certain embodiments, provided is a process for preparing a compound of Formula I, comprising:
contacting a compound of Formula I-4 with a compound of Formula I-5, under conditions suitable to provide a compound of Formula I.

In certain embodiments, provided is a process for preparing a compound of Formula I, comprising:
contacting a compound of Formula I-1 with a compound of Formula I-3, under conditions suitable to provide a compound of Formula I-4; and
contacting a compound of Formula I-4 with a compound of Formula I-5, under conditions suitable to provide a compound of Formula I.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### General Experimental Methods

All solvents used were commercially available and were used without further purification. Reactions were typically run using anhydrous solvents under an inert atmosphere of nitrogen.

NMR Spectroscopy: ¹H Nuclear magnetic resonance (NMR) spectroscopy was carried out using a Bruker Avance III equipped with a BBFO 300 MHz probe operating at 300 MHz or one of the following instruments: a Bruker Avance 400 instrument equipped with probe DUAL 400 MHz S1, a Bruker Avance 400 instrument equipped with probe 6 Si 400 MHz 5 mm ¹H-¹³C ID, a Bruker Avance III 400 instrument with nanobay equipped with probe Broadband BBFO 5 mm direct, a Bruker Mercury Plus 400 NMR spectrometer equipped with a Bruker 400 BBO probe operating at 400 MHz. All deuterated solvents contained typically 0.03% to 0.05% v/v tetramethylsilane, which was used as the reference signal (set at δ 0.00 for both ¹H and ¹³C). In certain cases, ¹H Nuclear magnetic resonance (NMR) spectroscopy was carried out using a Bruker Advance 400 instrument operating at 400 MHz using the stated solvent at around room temperature unless otherwise stated. In all cases, NMR data were consistent with the proposed structures. Characteristic chemical shifts (δ) are given in parts-per-million using conventional abbreviations for designation of major peaks: e.g. s, singlet; d, doublet; t, triplet; q, quartet; dd, doublet of doublets; dt, doublet of triplets; br, broad.

Thin Layer Chromatography: Where thin layer chromatography (TLC) has been used it refers to silica gel TLC using silica gel F254 (Merck) plates, Rf is the distance travelled by the compound divided by the distance travelled by the solvent on a TLC plate. Column chromatography was performed using an automatic flash chromatography system over silica gel cartridges or in the case of reverse phase chromatography over C18 cartridges. Alternatively, thin layer chromatography (TLC) was performed on Alugram® (Silica gel 60 F254) from Mancherey-Nagel and UV was typically used to visualize the spots. Additional visualization methods were also employed in some cases. In these cases the TLC plate was developed with iodine (generated by adding approximately 1 g of I₂ to 10 g silica gel and thoroughly mixing), ninhydrin (available commercially from Aldrich), or Magic Stain (generated by thoroughly mixing 25 g (NH₄)₆Mo₇O₂₄·4H₂O, 5 g (NH₄)₂Ce(IV)(NO₃)₆ in 450 mL water and 50 mL concentrated H₂SO₄) to visualize the compound.

Liquid Chromatography-Mass Spectrometry and HPLC Analysis: HPLC analysis was performed on Shimadzu 20AB HPLC system with a photodiode array detector and Luna-C18(2) 2.0×50 mm, 5 μm column at a flow rate of 1.2 mL/min with a gradient solvent Mobile phase A (MPA, H₂O+0.037% (v/v) TFA): Mobile phase B (MPB, ACN+0.018% (v/v) TFA) (0.01 min, 10% MPB; 4 min, 80% MPB; 4.9 min, 80% MPB; 4.92 min, 10% MPB; 5.5 min, 10% MPB). LCMS was detected under 220 and 254 nm or used evaporative light scattering (ELSD) detection as well as positive electrospray ionization (MS). Semi-preparative HPLC was performed by either acidic or neutral conditions. Acidic: Luna C18 100×30 mm, 5 μm; MPA: HCl/H₂O=0.04%, or formic acid/H₂O=0.2% (v/v); MPB: ACN. Neutral: Waters Xbridge 150×25, 5 μm; MPA: 10 mM NH₄HCO₃ in H₂O; MPB: ACN. Gradient for both conditions: 10% of MPB to 80% of MPB over 12 min at a flow rate of 20 mL/min, then 100% MPB over 2 min, 10% MPB over 2 min, UV detector. SFC analysis was performed on Thar analytical SFC system with a UV/Vis detector and series of chiral columns including AD, AS-H, OJ, OD, AY and IC, 4.6×100 mm, 3 μm column at a flow rate of 4 mL/min with a gradient solvent Mobile phase A (MPA, CO₂): Mobile phase B (MPB, MeOH+0.05% (v/v) IPAm) (0.01 min, 10% MPB; 3 min, 40% MPB; 3.5 min, 40% MPB; 3.56-5 min, 10% MPB). SFC preparative was performed on Thar 80 preparative SFC system with a UV/Vis detector and series of chiral preparative columns including AD-H, AS-H, OJ-H, OD-H, AY-H and IC-H, 30×250 mm, 5 μm column at a flow rate of 65 mL/min with a gradient solvent Mobile phase A (MPA, CO₂): Mobile phase B (MPB, MeOH+0.1% (v/v) NH₃H₂O) (0.01 min, 10% MPB; 5 min, 40% MPB; 6 min, 40% MPB; 6.1-10 min, 10% MPB). LC-MS data were also collected using an UPLC-MS Acquity™ system equipped with PDA detector and coupled to a Waters single quadrupole mass spectrometer operating in alternated positive and negative electrospray ionization mode. The column used was a Cortecs UPLC C18, 1.6 μm, 2.1×50 mm. A linear gradient was applied, starting at 95% A (A: 0.1% formic acid in water) and ending at 95% B (B: 0.1% formic acid in MeCN) over 2.0 min with a total run time of 2.5 min. The column temperature was at 40° C. with the flow rate of 0.8 mL/min.

### Intermediate 1

### 6-bromospiro[2,3-dihydroisoquinoline-4,1′-cyclopropane]-1-one

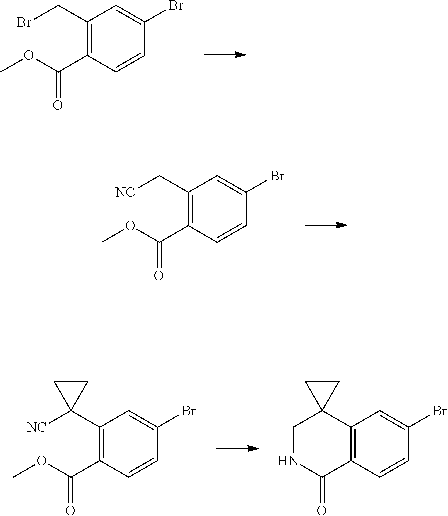 [SMILES: COC(=O)c1ccc(Br)cc1CBr ; COC(=O)c1ccc(Br)cc1CC#N ; COC(=O)c1ccc(Br)cc1C1(C#N)CC1 ; O=C1NCC2(CC2)c2cc(Br)ccc21]

Methyl 4-bromo-2-(cyanomethyl)benzoate: A solution of NaCN (8.4 g, 170 mmol) in H₂O (40 mL) was added dropwise to a solution of methyl 4-bromo-2-(bromomethyl)benzoate (35 g, 114 mmol) in DMSO (500 mL) at 0° C. The reaction mixture was stirred at 20° C. for 1 h. The reaction mixture was poured into H₂O (1.5 L) and extracted with EtOAc (3×200 mL). The combined organic layers were washed with brine (3×200 mL), dried over anhydrous Na₂SO₄, filtered, and concentrated under reduced pressure. The residue was purified by silica gel column chromatography. ¹H NMR (400 MHz, CDCl₃): δ 7.93 (d, J=8.4 Hz, 1H), 7.73 (d, J=2.0 Hz, 1H), 7.57 (dd, J=2.0, 8.8 Hz, 1H), 4.20 (s, 2H), 3.92 (s, 3H).

Methyl 4-bromo-2-(1-cyanocyclopropyl)benzoate: Methyl 4-bromo-2-(cyanomethyl)benzoate (9.0 g, 35.4 mmol) was added to a solution of NaH (3.26 g, 81.5 mmol, 60% purity) in DMSO (90 mL). The reaction mixture was stirred at 20° C. for 1 h before the addition of 1-bromo-2-chloroethane (8.13 g, 56.7 mmol). The resultant mixture was stirred at 20° C. for a further 8 h. The reaction mixture was cooled to 0° C., diluted with saturated aqueous NH₄Cl (200 mL), and extracted with EtOAc (4×50 mL). The combined organic layers were washed with brine (3×30 mL), dried over anhydrous Na₂SO₄, filtered, and concentrated under reduced pressure. The residue was purified by silica gel column chromatography. LCMS: m/z=280.0, 282.0 [M+H]⁺.

6-bromospiro[2,3-dihydroisoquinoline-4,1′-cyclopropane]-1-one: To a mixture of methyl 4-bromo-2-(1-cyanocyclopropyl)benzoate (2.50 g, 8.92 mmol) and dichlorocobalt (2.32 g, 17.9 mmol) in MeOH (40 mL) was added NaBH₄ (1.70 g, 44.9 mmol) at 0° C. The reaction mixture was stirred at 20° C. for 3 h. The reaction mixture was cooled to 0° C., diluted with saturated aqueous NH₄Cl (50 mL), and extracted with DCM (4×15 mL). The combined organic layers were washed with brine (20 mL), dried over anhydrous Na₂SO₄, filtered, and concentrated under reduced pressure. The residue was purified by silica gel column chromatography. LCMS: m/z=252.0, 254.0 [M+H]⁺.

### Intermediate 2

### Methyl 2-(6-bromo-1-oxo-spiro[3H-isoquinoline-4,1′-cyclopropane]-2-yl)acetate

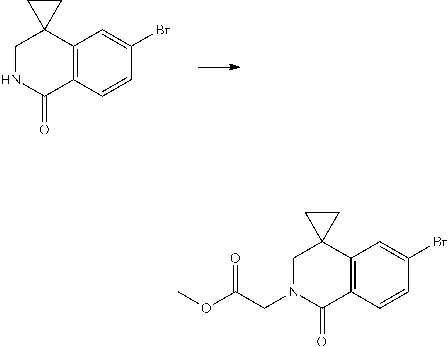 [SMILES: O=C1NCC2(CC2)c2cc(Br)ccc21 ; COC(=O)CN1CC2(CC2)c2cc(Br)ccc2C1=O]

To a solution of 6-bromospiro[2,3-dihydroisoquinoline-4,1′-cyclopropane]-1-one (2.30 g, 9.12 mmol) in DMF (30 mL) was added NaH (401 mg, 10.0 mmol, 60% purity) at 0° C. The reaction mixture was stirred at 0° C. for 0.5 h before the addition of methyl 2-bromoacetate (2.09 g, 13.7 mmol). The reaction mixture was then stirred at 20° C. for a further 2 h. The reaction mixture was cooled to 0° C., diluted with saturated aqueous NH₄Cl (50 mL), and extracted with EtOAc (4×20 mL). The combined organic layers were washed with brine (3×15 mL), dried over anhydrous Na₂SO₄, filtered, and concentrated under reduced pressure. The residue was purified by silica gel column chromatography. LCMS: m/z=324.0, 326.0 [M+H]⁺. ¹H NMR (400 MHz, CDCl₃): δ 7.98 (d, J=8.4 Hz, 1H), 7.44 (dd, J=2.0, 8.4 Hz, 1H), 6.99 (d, J=2.0 Hz, 1H), 4.33 (s, 2H), 3.76 (s, 3H), 3.45 (s, 2H), 1.14-1.09 (m, 2H), 1.08-1.03 (m, 2H).

### Intermediate 3

### 2-(6-bromo-1-oxo-spiro[3H-isoquinoline-4,1′-cyclopropane]-2-yl)acetic acid

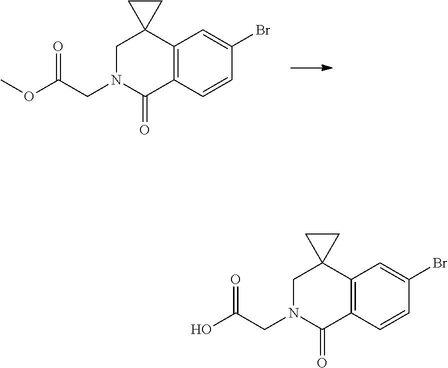 [SMILES: COC(=O)CN1CC2(CC2)c2cc(Br)ccc2C1=O ; O=C(O)CN1CC2(CC2)c2cc(Br)ccc2C1=O]

To a solution of methyl 2-(6-bromo-1-oxo-spiro[3H-isoquinoline-4,1′-cyclopropane]-2-yl)acetate (330 mg, 1.02 mmol) in THF (5 mL) was added a solution of LiOH (2.04 mL, 1 M in H₂O). The reaction mixture was stirred at 20° C. for 3 h. The reaction mixture was adjusted pH to 4 using aqueous 1 N HCl and extracted with EtOAc (3×10 mL). The combined organic layers were washed with brine, dried over anhydrous Na₂SO₄, filtered, and concentrated under reduced pressure to provide a residue that was used directly. LCMS: m/z=310.3, 312.3 [M+H]⁺.

### Intermediate 4

### 6-(trifluoromethyl)spiro[2,3-dihydroisoquinoline-4,1′-cyclopropane]-1-one

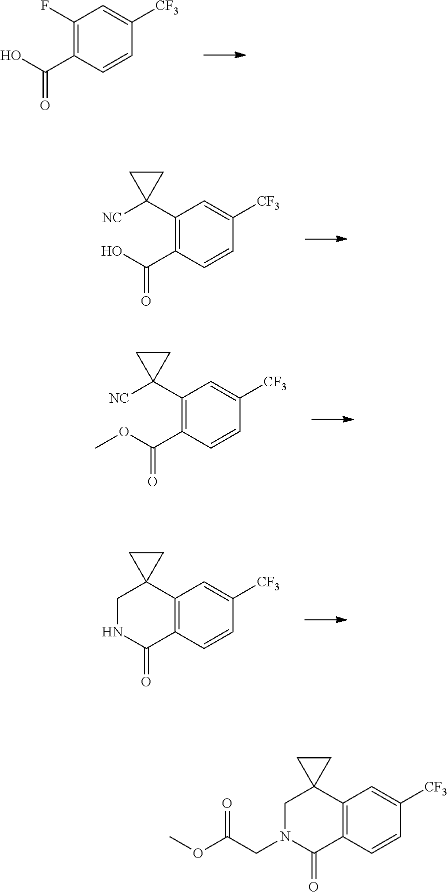 [SMILES: O=C(O)c1ccc(C(F)(F)F)cc1F ; N#CC1(c2cc(C(F)(F)F)ccc2C(=O)O)CC1 ; COC(=O)c1ccc(C(F)(F)F)cc1C1(C#N)CC1 ; O=C1NCC2(CC2)c2cc(C(F)(F)F)ccc21 ; COC(=O)CN1CC2(CC2)c2cc(C(F)(F)F)ccc2C1=O]

2-(1-cyanocyclopropyl)-4-(trifluoromethyl)benzoic acid: To a solution of 2-fluoro-4-(trifluoromethyl)benzoic acid (2.00 g, 9.61 mmol) and cyclopropanecarbonitrile (1.93 g, 28.8 mmol) in THF (20 mL) at −40° C. was added dropwise KHMDS (25.0 mL, 1 M in THF). The reaction mixture was slowly warmed up to 20° C. then stirred at this temperature for 1 h. The reaction mixture was then heated to 70° C., and stirred for an additional 2 h. The reaction mixture was poured into H₂O (20 mL) and washed with EtOAc (50 mL). The aqueous layer was then adjusted to pH=3-4 by the addition of aqueous HCl (3 N). The aqueous was then extracted with EtOAc (3×20 mL). The combined organics were dried over anhydrous Na₂SO₄, filtered, and concentrated under reduced pressure. The residue was purified by reverse-phase HPLC. ¹H NMR (400 MHz, CDCl₃): δ 8.26 (d, J=8.0 Hz, 1H), 7.82-7.72 (m, 2H), 1.92-1.84 (m, 2H), 1.46-1.38 (m, 2H).

Methyl 2-(1-cyanocyclopropyl)-4-(trifluoromethyl)benzoate: To a solution of 2-(1-cyanocyclopropyl)-4-(trifluoromethyl)benzoic acid (0.50 g, 1.96 mmol) in THF (10 mL) at 0° C. was added TMSCHN₂ (3.92 mmol, 2 M in n-hexane). The reaction mixture was stirred at 20° C. for 16 h. The reaction mixture was quenched by the addition of AcOH (2 mL) and the resulting mixture was extracted with EtOAc (20 mL). The organic layer was washed with saturated aqueous NaHCO₃ (10 mL), dried over anhydrous Na₂SO₄, filtered, and concentrated under reduced pressure. The residue was purified by silica gel column chromatography. ¹H NMR (400 MHz, CDCl₃): δ 8.08 (d, J=8.0 Hz, 1H), 7.75-7.67 (m, 2H), 4.11-3.99 (s, 3H), 1.84-1.78 (m, 2H), 1.38-1.32 (m, 2H).

6-(trifluoromethyl)spiro[2,3-dihydroisoquinoline-4,1′-cyclopropane]-1-one: To a mixture of methyl 2-(1-cyanocyclopropyl)-4-(trifluoromethyl)benzoate (0.30 g, 1.11 mmol) and dichlorocobalt (289 mg, 2.23 mmol) in MeOH (5 mL) and THF (2 mL) at 0° C. was added NaBH₄ (211 mg, 5.57 mmol). The reaction mixture was stirred at 20° C. for 2 h. The reaction mixture cooled to 0° C., quenched by the addition of sat. aq. NH₄Cl (10 mL), and extracted with EtOAc (3×10 mL). The organics were combined, dried over anhydrous Na₂SO₄, filtered and concentrated under reduced pressure to provide a residue that was used directly. LCMS: m/z=242.1 [M+H]⁺

Methyl 2-[1-oxo-6-(trifluoromethyl)spiro[3H-isoquinoline-4,1′-cyclopropane]-2-yl]acetate: To a solution of 6-(trifluoromethyl)spiro[2,3-dihydroisoquinoline-4,1′-cyclopropane]-1-one (0.20 g, 0.83 mmol) in DMF (5.0 mL) at 0° C. was added NaH (36 mg, 0.91 mmol, 60% purity in mineral oil). The reaction mixture was stirred at 0° C. for 0.5 h followed by the addition of methyl 2-bromoacetate (190 mg, 1.24 mmol). The reaction mixture was stirred at 20° C. for a further 2 h. The reaction mixture was quenched by the addition of water (5 mL) and extracted with EtOAc (3×5 mL). These organics were combined, dried over anhydrous Na₂SO₄, filtered, and concentrated under reduced pressure. The residue was purified by silica gel column chromatography. ¹H NMR (400 MHz, CDCl₃): δ 8.23 (d, J=8.0 Hz, 1H), 7.55 (d, J=8.0 Hz, 1H), 7.08 (s, 1H), 4.35 (s, 2H), 3.76 (s, 3H), 3.48 (s, 2H), 1.21-1.06 (m, 4H).

### Intermediate 5

### Methyl 2-(6-bromo-1-oxospiro[3H-isoquinoline-4,1′-cyclobutane]-2-yl)acetate

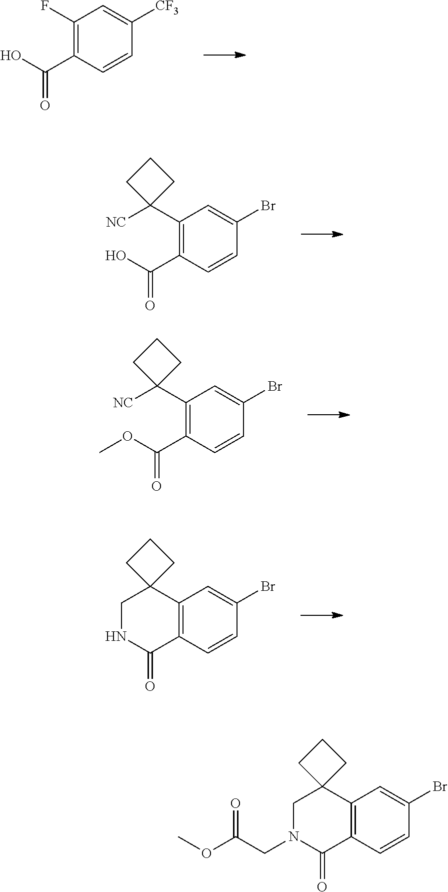

4-bromo-2-(1-cyanocyclobutyl)benzoic acid: To a solution of 4-bromo-2-fluorobenzoic acid (1.0 g, 4.57 mmol) and cyclobutanecarbonitrile (1.11 g, 13.7 mmol) in THF (10 mL) at −40° C. was added KHMDS (11.88 mL, 1 M in THF). The reaction mixture was stirred at 25° C. for 1 h and then stirred at 40° C. for an additional 2 h. The reaction mixture was diluted with water (10 mL) and extracted with EtOAc (2×8 mL). The organic layers were discarded and the aqueous layer was adjusted to pH=3 with aqueous HCl (3 N). The aqueous was then extracted with EtOAc (3×8 mL). The organics were combined, washed with brine (20 mL), dried over anhydrous Na₂SO₄, filtered, and concentrated under reduced pressure to provide a residue that was used directly. ¹H NMR (400 MHz, CDCl₃): δ 7.95 (d, J=8.4 Hz, 1H), 7.60-7.58 (m, 1H), 7.52 (d, J=2.0 Hz, 1H) 2.65-2.38 (m, 6H).

Methyl 4-bromo-2-(1-cyanocyclobutyl)benzoate: To a solution of 4-bromo-2-(1-cyanocyclobutyl)benzoic acid (1.0 g, 3.57 mmol) in THF (8 mL) and MeOH (2 mL) at 0° C. was added TMSCHN₂ (3.57 mL, 2 M in n-hexane). The reaction mixture was stirred at 25° C. for 2 h. The reaction mixture was quenched by addition of sat. aq. Na₂S₂O₃ (50 mL) and extracted with EtOAc (3×50 mL). The combined organic layers were washed with brine (50 mL), dried over anhydrous Na₂SO₄, filtered, and concentrated under reduced pressure. The residue was purified by silica gel column chromatography. ¹H NMR (400 MHz, CDCl₃): δ 7.45 (d, J=8.4 Hz, 1H), 7.54-7.51 (m, 1H), 7.47 (d, J=2.0 Hz, 1H), 3.92 (s, 3H), 2.94-2.89 (m, 2H), 2.56-2.45 (m, 2H), 1.95-1.92 (m, 2H).

6-bromospiro[2,3-dihydroisoquinoline-4,1′-cyclobutane]-1-one: To a solution of methyl 4-bromo-2-(1-cyanocyclobutyl)benzoate (360 mg, 1.22 mmol) and dichlorocobalt (318 mg, 2.45 mmol) in MeOH (6 mL) at 0° C. was added NaBH₄ (278 mg, 7.34 mmol). The reaction mixture was stirred at 20° C. for 3 h. The reaction mixture was cooled to 0° C., diluted with sat. aq. NH₄Cl (30 mL), and extracted with DCM (4×30 mL). The combined organic layers were washed with brine (30 mL), dried over anhydrous Na₂SO₄, filtered, and concentrated under reduced pressure. The residue was purified by silica gel column chromatography. ¹H NMR (400 MHz, CDCl₃): δ 7.93 (d, J=8.4 Hz, 1H), 7.65 (d, J=1.6 Hz, 1H) 7.55-7.49 (m, 1H), 3.59-3.58 (m, 2H), 2.34-2.31 (m, 2H), 2.18-2.08 (m, 4H).

Methyl 2-(6-bromo-1-oxospiro[3H-isoquinoline-4,1′-cyclobutane]-2-yl)acetate: To a solution of 6-bromospiro[2,3-dihydroisoquinoline-4,1′-cyclobutane]-1-one (150 mg, 0.56 mmol) and methyl 2-bromoacetate (95 mg, 0.62 mmol) in DMF (1.5 mL) was added Cs₂CO₃ (276 mg, 0.845 mmol). The reaction mixture was stirred at 70° C. for 12 h. The reaction mixture was diluted with water (10 mL) and extracted with EtOAc (3×10 mL). The combined organic layers were washed with brine (10 mL), dried over anhydrous Na₂SO₄, filtered, and concentrated under reduced pressure. The residue was purified by silica gel column chromatography. ¹H NMR (400 MHz, CDCl₃): δ 7.95 (d, J=8.4 Hz, 1H), 7.64 (d, J=2.0 Hz, 1H), 7.53-7.47 (m, 1H). 4.36 (s, 1H), 4.25 (s, 1H), 3.78-3.77 (m, 3H), 3.69-3.68 (m, 2H), 2.38-2.33 (m, 2H), 2.22-2.07 (m, 4H).

### Intermediate 6

### Methyl 2-[2′-methyl-1-oxo-6-(trifluoromethyl)spiro[3H-isoquinoline-4,1′-cyclopropane]-2-yl]acetate

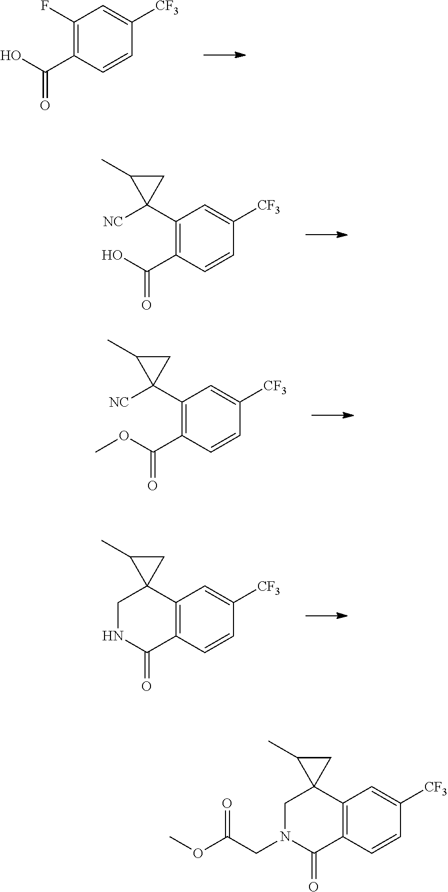 [SMILES: O=C(O)c1ccc(C(F)(F)F)cc1F ; CC1CC1(C#N)c1cc(C(F)(F)F)ccc1C(=O)O ; COC(=O)c1ccc(C(F)(F)F)cc1C1(C#N)CC1C ; CC1CC12CNC(=O)c1ccc(C(F)(F)F)cc12 ; COC(=O)CN1CC2(CC2C)c2cc(C(F)(F)F)ccc2C1=O]

2-(1-cyano-2-methylcyclopropyl)-4-(trifluoromethyl)benzoic acid: To a solution of 2-fluoro-4-(trifluoromethyl)benzoic acid (2.00 g, 9.61 mmol) and 2-methylcyclopropanecarbonitrile (2.34 g, 28.8 mmol) in THF (20 mL) at −40° C. was added KHMDS (38.4 mmol, 1 M in THF, 38.44 mL). The reaction mixture was stirred at 25° C. for 0.5 h then stirred at 50° C. for a further 1 h. The reaction mixture was diluted with water (10 mL) and extracted with EtOAc (2×8 mL). The organic layers were discarded and the aqueous layer was adjusted to pH=3 with aqueous HCl (3 N). The aqueous was then extracted with EtOAc (3×8 mL). The combined organics were dried over anhydrous Na₂SO₄, filtered, and concentrated under reduced pressure to give a residue that was used directly. LCMS: m/z=268.1 [M−H]⁻.

Methyl 2-(1-cyano-2-methylcyclopropyl)-4-(trifluoromethyl)benzoate: To a solution of 2-(1-cyano-2-methylcyclopropyl)-4-(trifluoromethyl)benzoic acid (2.50 g, 9.29 mmol) in THF (20 mL) and MeOH (5 mL) at 0° C. was added TMSCHN₂ (9.29 mL, 2 M in n-hexane). The reaction mixture was stirred at 25° C. for 1 h. The reaction mixture was diluted with water (50 mL) and extracted with EtOAc (4×50 mL). The combined organic layers were washed with brine (30 mL), dried over anhydrous Na₂SO₄, filtered, and concentrated under reduced pressure. The residue was purified by silica gel column chromatography. LCMS: m/z=284.0 [M+H]⁺.

2′-methyl-6-(trifluoromethyl)spiro[2,3-dihydroisoquinoline-4,1′-cyclopropane]-1-one: To a solution of methyl 2-(1-cyano-2-methylcyclopropyl)-4-(trifluoromethyl)benzoate (1.00 g, 3.53 mmol) in MeOH (20 mL) at 0° C. were added NaBH₄ (801 mg, 21.2 mmol) and dichlorocobalt (917 mg, 7.06 mmol). The reaction mixture was stirred at 25° C. for 2 h. The reaction mixture was cooled to 0° C., diluted with sat. aq. NH₄Cl (50 mL), and extracted with EtOAc (4×50 mL). The combined organics were washed with brine (30 mL), dried over anhydrous Na₂SO₄, filtered, and concentrated under reduced pressure. The residue was purified by silica gel column chromatography. ¹H NMR (400 MHz, DMSO-d₆): δ 8.25 (br s, 1H), 8.04 (d, J=8.0 Hz, 1H), 7.63 (d, J=8.0 Hz, 1H), 7.27 (s, 1H), 3.53-3.49 (m, 1H), 3.23-3.20 (m, 1H), 1.54-1.47 (m, 1H), 1.22 (s, 3H), 1.98-1.92 (m, 1H), 0.76-0.68 (m, 1H).

Methyl 2-[2′-methyl-1-oxo-6-(trifluoromethyl)spiro[3H-isoquinoline-4,1′-cyclopropane]-2-yl]acetate: To a solution of 2′-methyl-6-(trifluoromethyl)spiro[2,3-dihydroisoquinoline-4,1′-cyclopropane]-1-one (0.4 g, 1.57 mmol) and methyl 2-bromoacetate (239.74 mg, 1.57 mmol) in DMF (5 mL) was added Cs₂CO₃ (765.93 mg, 2.35 mmol). The reaction mixture was stirred at 50° C. for 10 h. The reaction mixture was quenched with sat. aq. NH₄Cl (50 mL) at 0° C., and extracted with EtOAc (4×50 mL). The combined organic layers were washed with brine (30 mL), dried over anhydrous Na₂SO₄, filtered, and concentrated under reduced pressure to give a residue. The residue was purified by silica gel column chromatography. ¹H NMR (400 MHz, DMSO-d₆): δ 8.07 (d, J=8.0 Hz, 1H), 7.66 (d, J=8.0 Hz, 1H), 7.32 (s, 1H), 4.40-4.26 (m, 2H), 3.78 (d, J=13.2 Hz, 1H), 3.68 (s, 3H), 3.53 (d, J=13.2 Hz, 1H), 1.53-1.50 (m, 1H), 1.35-1.32 (m, 1H), 1.27 (d, J=2.4 Hz, 3H), 0.78-0.75 (m, 1H).

### Intermediate 7

### Methyl 2-[4-ethyl-4-methyl-1-oxo-6-(trifluoromethyl)-3H-isoquinolin-2-yl]acetate

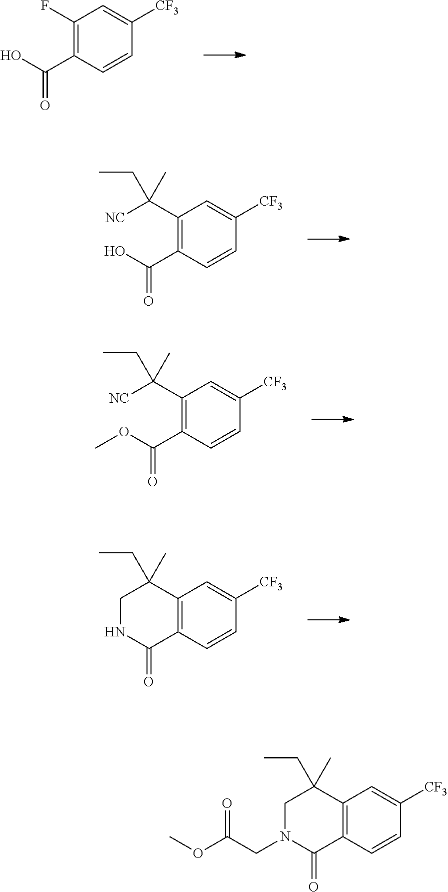 [SMILES: O=C(O)c1ccc(C(F)(F)F)cc1F ; CCC(C)(C#N)c1cc(C(F)(F)F)ccc1C(=O)O ; CCC(C)(C#N)c1cc(C(F)(F)F)ccc1C(=O)OC ; CCC1(C)CNC(=O)c2ccc(C(F)(F)F)cc21 ; CCC1(C)CN(CC(=O)OC)C(=O)c2ccc(C(F)(F)F)cc21]

2-(2-cyanobutan-2-yl)-4-(trifluoromethyl)benzoic acid: To a solution of 2-fluoro-4-(trifluoromethyl)benzoic acid (7.00 g, 33.6 mmol) and 2-methylbutanenitrile (16.8 g, 201 mmol) in THF (70 mL) at −40° C. was added KHMDS (175 mL, 1 M in THF). The reaction mixture was stirred at 25° C. for 1 h then stirred at 40° C. for a further 2 h. The reaction mixture was diluted with water (100 mL) and extracted with EtOAc (3×10 mL). The aqueous was lyophilized to give a residue that was used directly. LCMS: m/z=270.2 [M−H]⁻.

Methyl 2-(2-cyanobutan-2-yl)-4-(trifluoromethyl) benzoate: To a solution of 2-(2-cyanobutan-2-yl)-4-(trifluoromethyl) benzoic acid (5.00 g, 18.4 mmol) in DMF (50 mL) at 0° C. were added K₂CO₃ (3.82 g, 27.7 mmol) and CH₃I (3.92 g, 27.7 mmol). The reaction mixture was stirred at 25° C. for 1 h. The reaction mixture was diluted with sat. aq. NaHCO₃ (50 mL) and extracted with EtOAc (3×50 mL). The combined organics were washed with brine (50 mL), dried over anhydrous Na₂SO₄, filtered, and concentrated under reduced pressure. The residue was purified by silica gel column chromatography. LCMS: m/z=286.0 [M+H]⁺.

4-ethyl-4-methyl-6-(trifluoromethyl)-2,3-dihydroisoquinolin-1-one: To a solution of methyl 2-(2-cyanobutan-2-yl)-4-(trifluoromethyl)benzoate (800 mg, 2.80 mmol) in MeOH (16 mL) at 0° C. were added NaBH₄ (637 mg, 16.8 mmol) and dichlorocobalt (364 mg, 2.80 mmol). The reaction mixture was stirred at 25° C. for 2 h. The reaction mixture was cooled to 0° C., diluted with sat. aq. NH₄Cl(15 mL), and extracted with EtOAc (3×15 mL). The combined organic layers were washed with brine (15 mL), dried over anhydrous Na₂SO₄, filtered, and concentrated under reduced pressure. The residue was purified by silica gel column chromatography. ¹H NMR (400 MHz, CDCl₃): δ 8.21 (d, J=8.0 Hz, 1H), 7.62 (d, J=8.0 Hz, 1H), 7.52 (s, 1H), 6.78 (br s, 1H), 3.48-3.33 (m, 2H), 1.84-1.64 (m, 2H), 1.36 (s, 3H), 0.85 (t, J=7.2 Hz, 3H).

Methyl 2-[4-ethyl-4-methyl-1-oxo-6-(trifluoromethyl)-3H-isoquinolin-2-yl]acetate: To a solution of 4-ethyl-4-methyl-6-(trifluoromethyl)-2,3-dihydroisoquinolin-1-one (200 mg, 0.78 mmol) in DMF (2 mL) were added methyl 2-bromoacetate (178 mg, 1.17 mmol) and Cs₂CO₃ (507 mg, 1.55 mmol). The reaction mixture was stirred at 40° C. for 2 h. The reaction mixture was diluted with water (10 mL) and extracted with EtOAc (3×10 mL). The combined organics were washed with brine (10 mL), dried over anhydrous Na₂SO₄, filtered, and concentrated under reduced pressure. The residue was purified by silica gel preparatory TLC. LCMS: m/z=330.1 [M+H]⁺.

### Intermediate 8

### 2-(trifluoromethyl)spiro[6,7-dihydro-1,6-naphthyridine-8,1′-cyclopropane]-5-one

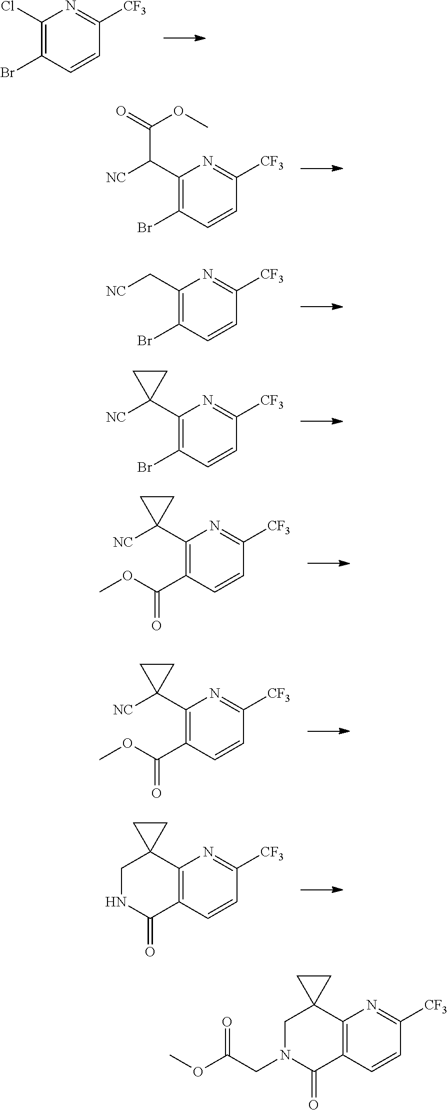

Methyl 2-[3-bromo-6-(trifluoromethyl)-2-pyridyl]-2-cyano-acetate: To a solution of 3-bromo-2-chloro-6-(trifluoromethyl)pyridine (2.00 g, 7.68 mmol) in CH₃CN (20 mL) were added methyl 2-cyanoacetate (1.52 g, 15.4 mmol) and Cs₂CO₃ (7.51 g, 23.0 mmol). The reaction mixture was stirred at 70° C. for 12 h. The reaction mixture was diluted with water (100 mL) and extracted with EtOAc (3×80 mL). The combined organics were washed with brine (100 mL), dried over anhydrous Na₂SO₄, filtered, and concentrated under reduced pressure. The residue was purified by silica gel column chromatography. ¹H NMR (400 MHz, CDCl₃) δ 8.17 (d, J=8.0 Hz, 1H), 7.64 (d, J=8.4 Hz, 1H), 5.44 (s, 1H), 3.90 (s, 3H).

2-(3-bromo-6-(trifluoromethyl)pyridin-2-yl)acetonitrile: To a solution of methyl 2-[3-bromo-6-(trifluoromethyl)-2-pyridyl]-2-cyano-acetate (1.60 g, 4.75 mmol) in DMSO (16 mL) was added a solution of NaCl (2.77 g, 47.5 mmol) in water (16 mL). The reaction mixture was stirred at 120° C. for 12 h. The reaction mixture was cooled to ambient temperature, diluted with water (80 mL) and extracted with EtOAc (3×60 mL). The combined organics were washed with brine (100 mL), dried over anhydrous Na₂SO₄, filtered, and concentrated under reduced pressure to provide a residue that was used directly. ¹H NMR (400 MHz, CDCl₃) δ 8.17 (d, J=8.0 Hz, 1H), 7.64 (d, J=8.0 Hz, 1H), 4.14 (s, 2H).

1-(3-bromo-6-(trifluoromethyl)pyridin-2-yl)cyclopropanecarbonitrile: To a mixture of 2-(3-bromo-6-(trifluoromethyl)pyridin-2-yl)acetonitrile (900 mg, 3.40 mmol) in DMF (10 mL) was added NaH (408 mg, 10.2 mmol, 60% purity). The reaction mixture was stirred at 25° C. for 15 min followed by the addition of 1,2-dibromoethane (1.91 g, 10.2 mmol). The reaction mixture was stirred at 25° C. for a further 15 min. The reaction mixture was diluted with water (40 mL) and extracted with EtOAc (3×60 mL). The combined organics were washed with brine (50 mL), dried over anhydrous Na₂SO₄, filtered, and concentrated under reduced pressure. The residue was purified by silica gel column chromatography. ¹H NMR (400 MHz, CDCl₃) δ 8.14 (d, J=8.0 Hz, 1H), 7.55 (d, J=8.0 Hz, 1H), 1.85-1.82 (m, 2H), 1.74-1.69 (m, 2H).

Methyl 2-(1-cyanocyclopropyl)-6-(trifluoromethyl)nicotinate: To a solution of 1-(3-bromo-6-(trifluoromethyl)pyridin-2-yl)cyclopropanecarbonitrile (900 mg, 3.09 mmol) in MeOH (30 mL) were added Pd(dppf)Cl₂ (113 mg, 0.15 mmol) and DIPEA (1.20 g, 9.28 mmol). The reaction mixture was stirred at 80° C. for 12 h under an atmosphere of CO (50 psi). The reaction mixture was diluted with water (120 mL) and extracted with EtOAc (3×80 mL). The combined organic layers were washed with brine (150 mL), dried over anhydrous Na₂SO₄, filtered, and concentrated under reduced pressure. The residue was purified by silica gel column chromatography. ¹H NMR (400 MHz, CDCl₃) δ 8.33 (d, J=8.0 Hz, 1H), 7.70 (d, J=8.0 Hz, 1H), 4.06 (s, 3H), 1.94-1.87 (m, 2H), 1.82-1.76 (m, 2H).

2-(trifluoromethyl)spiro[6,7-dihydro-1,6-naphthyridine-8,1′-cyclopropane]-5-one: To a solution of methyl 2-(1-cyanocyclopropyl)-6-(trifluoromethyl)nicotinate (600 mg, 2.22 mmol) in MeOH (12 mL) at 0° C. were added dichlorocobalt (577 mg, 4.44 mmol) and NaBH₄ (504 mg, 13.3 mmol). The reaction mixture was stirred at 25° C. for 1 h. The reaction mixture was cooled to 0° C., diluted with sat. aq. NH₄Cl (40 mL), and extracted with EtOAc (3×30 mL). The combined organics were washed with brine (50 mL), dried over anhydrous Na₂SO₄, filtered, and concentrated under reduced pressure. The residue was purified by preparatory TLC. ¹H NMR (400 MHz, CDCl₃) δ 8.49 (d, J=8.0 Hz, 1H), 7.60 (d, J=8.0 Hz, 1H), 6.66 (br s, 1H), 3.56 (d, J=4.0 Hz, 2H), 1.60-1.55 (m, 2H), 1.08-1.02 (m, 2H).

Methyl 2-[5-oxo-2-(trifluoromethyl)spiro[7H-1,6-naphthyridine-8,1′-cyclopropane]-6-yl]acetate: To a solution of 2-(trifluoromethyl)spiro[6,7-dihydro-1,6-naphthyridine-8,1′-cyclopropane]-5-one (240 mg, 0.99 mmol) in DMF (3.0 mL) were added methyl 2-bromoacetate (136 mg, 0.89 mmol) and Cs₂CO₃ (646 mg, 1.98 mmol). The reaction mixture was stirred at 40° C. for 2 h. The reaction mixture was diluted with water (10 mL) and extracted with EtOAc (3×8 mL). The combined organics were washed with brine (15 mL), dried over anhydrous Na₂SO₄, filtered, and concentrated under reduced pressure. The residue was purified by preparatory TLC. ¹H NMR (400 MHz, CDCl₃) δ 8.50 (d, J=8.0 Hz, 1H), 7.59 (d, J=8.0 Hz, 1H), 4.36 (s, 2H), 3.78 (s, 3H), 3.62 (s, 2H), 1.62-1.58 (m, 2H), 1.10-1.05 (m, 2H).

### Intermediate 9

### Methyl 2-[6-(difluoromethoxy)-1-oxospiro[3H-isoquinoline-4,1′-cyclopropane]-2-yl]acetate

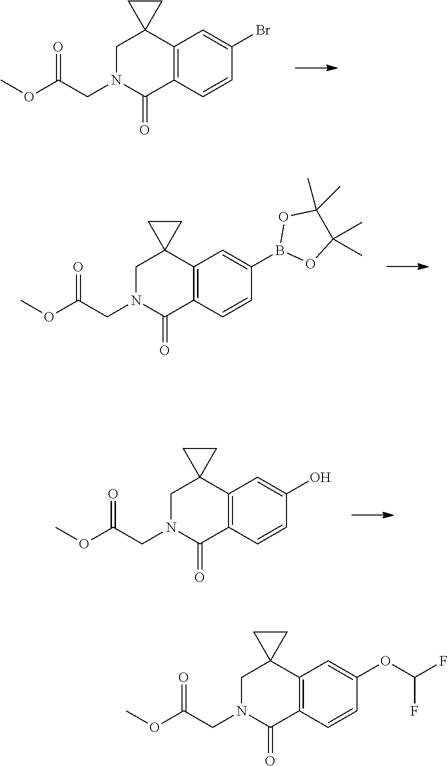

methyl 2-[1-oxo-6-(4,4,5,5-tetramethyl-1,3,2-dioxaborolan-2-yl)spiro[3H-isoquinoline-4,1′-cyclopropane]-2-yl]acetate: To a solution of methyl 2-(6-bromo-1-oxo-spiro[3H-isoquinoline-4,1′-cyclopropane]-2-yl)acetate (0.50 g, 1.54 mmol) and 4,4,4′,4′,5,5,5′,5′-octamethyl-2,2′-bi(1,3,2-dioxaborolane) (588 mg, 2.31 mmol) in 1,4-dioxane (10 mL) were added KOAc (454 mg, 4.63 mmol) and Pd(dppf)Cl₂ (12 mg, 0.02 mmol). The reaction mixture was stirred at 80° C. for 5 h. The reaction mixture was filtered and the filtrate was concentrated under reduced pressure. The residue was purified by silica gel column chromatography. LCMS: m/z=372.2 [M+H]⁺.

Methyl 2-(6-hydroxy-1-oxospiro[3H-isoquinoline-4,1′-cyclopropane]-2-yl)acetate: To a solution of methyl 2-[1-oxo-6-(4,4,5,5-tetramethyl-1,3,2-dioxaborolan-2-yl)spiro[3H-isoquinoline-4,1′-cyclopropane]-2-yl]acetate (670 mg, 1.80 mmol) in 1,4-dioxane (6 mL) and H₂O (6 mL) at 0° C. was added Oxone (1.22 g, 1.99 mmol). The reaction mixture was stirred at 20° C. for 4 h. The reaction mixture was diluted with sat. aq. Na₂S₂O₃ (10 mL) and extracted with EtOAc (3×10 mL). The combined organics were washed with brine (10 mL), dried over anhydrous Na₂SO₄, filtered, and concentrated under reduced pressure to give a residue that was used directly. LCMS: m/z=262.3 [M+H]⁺.

Methyl 2-[6-(difluoromethoxy)-1-oxospiro[3H-isoquinoline-4,1′-cyclopropane]-2-yl]acetate: To a solution of methyl 2-(6-hydroxy-1-oxospiro[3H-isoquinoline-4,1′-cyclopropane]-2-yl)acetate (390 mg, 1.49 mmol) and sodium 2-chloro-2,2-difluoroacetate (273 mg, 1.79 mmol) in DMF (5 mL) was added K₂CO₃ (413 mg, 2.99 mmol). The reaction mixture was stirred at 110° C. for 16 h. The reaction mixture was cooled to ambient temperature, diluted with water (5 mL), and extracted with EtOAc (3×5 mL). The combined organics were washed with brine (5 mL), dried over anhydrous Na₂SO₄, filtered, and concentrated under reduced pressure. The residue was purified by silica gel column chromatography. LCMS: m/z=312.1 [M+H]⁺.

### Intermediate 10

### Methyl 2-(6-cyclopropyl-1-oxospiro[3H-isoquinoline-4,1′-cyclopropane]-2-yl)acetate

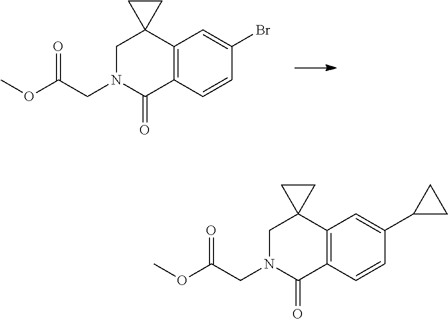 [SMILES: COC(=O)CN1CC2(CC2)c2cc(Br)ccc2C1=O ; COC(=O)CN1CC2(CC2)c2cc(C3CC3)ccc2C1=O]

To a solution of methyl 2-(6-bromo-1-oxo-spiro[3H-isoquinoline-4,1′-cyclopropane]-2-yl)acetate (200 mg, 0.62 mmol) in 1,4-dioxane (3.0 mL) were added cyclopropylboronic acid (159 mg, 1.85 mmol), CsF (282 mg, 1.85 mmol), and Pd(dppf)Cl₂ (45 mg, 0.06 mmol). The reaction mixture was stirred at 100° C. for 6 h. The reaction mixture was diluted with water (10 mL) and extracted with EtOAc (3×3 mL). The combined organic layers were washed with brine (10 mL), dried over anhydrous Na₂SO₄, filtered, and concentrated under reduced pressure. The residue was purified by silica gel column chromatography. LCMS: m/z=286.2 [M+H]⁺.

### Intermediate 11

### Methyl 2-[5-fluoro-1-oxo-6-(trifluoromethyl)spiro[3H-isoquinoline-4,1′-cyclopropane]-2-yl]acetate

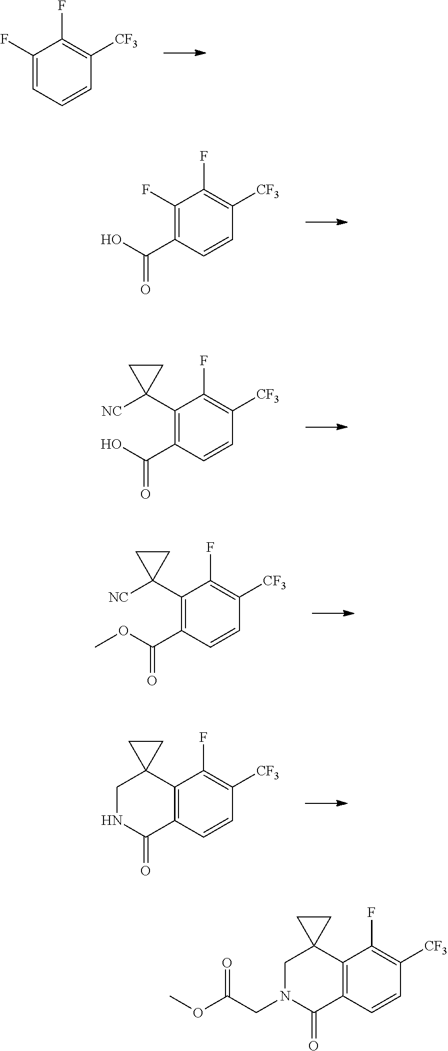 [SMILES: Fc1cccc(C(F)(F)F)c1F ; O=C(O)c1ccc(C(F)(F)F)c(F)c1F ; N#CC1(c2c(C(=O)O)ccc(C(F)(F)F)c2F)CC1 ; COC(=O)c1ccc(C(F)(F)F)c(F)c1C1(C#N)CC1 ; O=C1NCC2(CC2)c2c1ccc(C(F)(F)F)c2F ; COC(=O)CN1CC2(CC2)c2c(ccc(C(F)(F)F)c2F)C1=O]

2,3-difluoro-4-(trifluoromethyl)benzoic acid: To a solution of 1,2-difluoro-3-(trifluoromethyl)benzene (3.60 g, 19.8 mmol) in THF (240 mL) at −70° C. was added LDA (11.86 mL, 2 M in 12:25 THF:n-hexane). The reaction mixture was stirred at −70° C. for 2 h. Into the reaction mixture was added dry ice pellets (5 g) and the reaction mixture was stirred at −70° C. for a further 2 h. The reaction mixture was diluted with water (100 mL) and extracted with EtOAc (3×100 mL). The combined organics were dried over anhydrous Na₂SO₄, filtered, and concentrated under reduced pressure to provide a residue that was used directly. ¹H NMR (400 MHz, CDCl₃): δ 9.03 (br s, 1H), 7.78 (t, J=6.8 Hz, 1H), 7.41 (t, J=6.8 Hz, 1H).

2-(1-cyanocyclopropyl)-3-fluoro-4-(trifluoromethyl)benzoic acid: To a solution of 2,3-difluoro-4-(trifluoromethyl)benzoic acid (4.70 g, 20.8 mmol) and cyclopropanecarbonitrile (1.39 g, 20.8 mmol) in THF (100 mL) at −40° C. was added dropwise KHMDS (54.1 mL, 1 M in THF). The reaction mixture was stirred at −40° C. for 3 h. The reaction mixture was diluted with water (100 mL) and extracted with EtOAc (3×50 mL). The organics were discarded and the aqueous layer was adjusted pH=3-4 with aqueous HCl (2 N). The aqueous was extracted with EtOAc (3×50 mL). The combined organics were dried over anhydrous Na₂SO₄, filtered, and concentrated under reduced pressure to provide a residue that was used directly. ¹H NMR (400 MHz, CDCl₃): δ 9.34 (br s, 1H), 7.87-7.94 (m, 1H), 7.79-7.73 (m, 1H), 1.88-1.97 (m, 2H), 1.31-1.37 (m, 2H).

Methyl 2-(1-cyanocyclopropyl)-3-fluoro-4-(trifluoromethyl)benzoate: To a solution of 2-(1-cyanocyclopropyl)-3-fluoro-4-(trifluoromethyl)benzoic acid (4.50 g, 16.5 mmol) in DMF (50 mL) were added K₂CO₃ (3.41 g, 24.7 mmol) and CH₃I (2.57 g, 18.1 mmol). The reaction mixture was stirred at 20° C. for 2 h. The reaction mixture was diluted with EtOAc (100 mL) and washed with H₂O (50 mL) and brine (50 mL). The organic layer was dried over anhydrous Na₂SO₄, filtered, and concentrated under reduced pressure. The residue was purified by silica gel column chromatography. ¹H NMR (400 MHz, CDCl₃): δ 7.78-7.67 (m, 2H), 4.04 (s, 3H), 1.81-1.89 (m, 2H), 1.22-1.30 (m, 2H).

5-fluoro-6-(trifluoromethyl)spiro[2,3-dihydroisoquinoline-4,1′-cyclopropane]-1-one: To a mixture of methyl 2-(1-cyanocyclopropyl)-3-fluoro-4-(trifluoromethyl)benzoate (0.50 g, 1.74 mmol) and dichlorocobalt (226 mg, 1.74 mmol) in MeOH (10 mL) at 0° C. was added NaBH₄ (132 mg, 3.48 mmol). The reaction mixture was stirred at 20° C. for 2 h. The reaction mixture was cooled to 0° C., diluted with sat. aq. NH₄Cl (10 mL) and extracted with EtOAc (3×10 mL). The combined organics were washed with brine (50 mL), dried over anhydrous Na₂SO₄, filtered, and concentrated under reduced pressure to provide a residue that was used directly. LCMS: m/z=260.1 [M+H]⁺.

Methyl 2-[5-fluoro-1-oxo-6-(trifluoromethyl)spiro[3H-isoquinoline-4,1′-cyclopropane]-2-yl]acetate: To a solution of 5-fluoro-6-(trifluoromethyl)spiro[2,3-dihydroisoquinoline-4,1′-cyclopropane]-1-one (0.29 g, 1.12 mmol) in DMF (5 mL) at 0° C. was added NaH (49 mg, 1.23 mmol, 60% purity). The reaction mixture was stirred at 0° C. for 0.5 h followed by the addition of methyl 2-bromoacetate (257 mg, 1.68 mmol). The reaction mixture was stirred at 20° C. for a further 2 h. The reaction mixture was diluted with water (5 mL) and extracted with EtOAc (3×5 mL). The combined organic layers were dried over anhydrous Na₂SO₄, filtered, and concentrated under reduced pressure. The residue was purified by silica gel column chromatography. ¹H NMR (400 MHz, CDCl₃): δ 8.05 (d, J=8.0 Hz, 1H), 7.54 (d, J=8.0 Hz, 1H), 4.34 (s, 2H), 3.78 (s, 3H), 3.40 (s, 2H), 1.63-1.70 (m, 2H), 1.02-1.09 (m, 2H).

### Intermediate 12

### 2-(6-bromo-4,4-dimethyl-1-oxo-3H-isoquinolin-2-yl)acetic acid

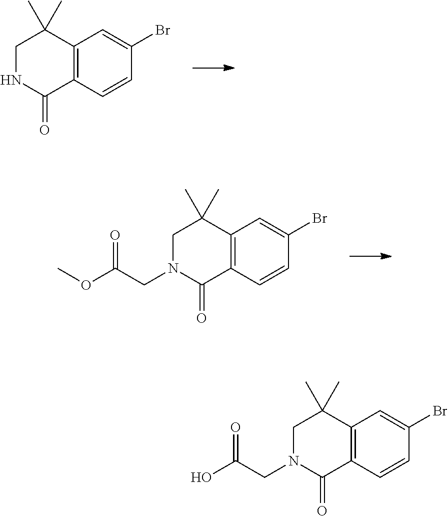 [SMILES: CC1(C)CNC(=O)c2ccc(Br)cc21 ; COC(=O)CN1CC(C)(C)c2cc(Br)ccc2C1=O ; CC1(C)CN(CC(=O)O)C(=O)c2ccc(Br)cc21]

Methyl 2-(6-bromo-4,4-dimethyl-1-oxo-3H-isoquinolin-2-yl)acetate: To a mixture of 6-bromo-4,4-dimethyl-2,3-dihydroisoquinolin-1-one (182 mg, 0.72 mmol) and Cs₂CO₃ (352 mg, 1.07 mmol) in MeCN (2.9 mL) were added methyl bromoacetate (131 mg, 0.86 mmol) and tetrabutylammonium iodide (26 mg, 0.07 mmol). The reaction mixture was stirred at 80° C. for 16 h. The reaction mixture was cooled to ambient temperature and diluted with EtOAc (15 mL). The resulting mixture was filtered through a pad of silica and the filtrate was concentrated under reduced pressure. The residue was purified by silica gel column chromatography. LCMS: m/z=326.2, 328.2 [M+H]⁺.

2-(6-bromo-4,4-dimethyl-1-oxo-3H-isoquinolin-2-yl)acetic acid: To a solution of methyl 2-(6-bromo-4,4-dimethyl-1-oxo-3H-isoquinolin-2-yl)acetate (234 mg, 0.72 mmol) in THF (15 mL) was added LiOH (0.72 mL, 2 M in water). The reaction mixture was stirred at 23° C. for 3 h. The reaction mixture was adjusted pH=3-4 using aqueous HCl (1 N) and extracted with EtOAc (3×10 mL). The combined organics were washed with brine, dried over anhydrous Na₂SO₄, filtered, and concentrated under reduced pressure to provide a residue that was used directly. LCMS: m/z=312.1, 314.1 [M+H]⁺.

### Intermediate 13

### 2-(6-bromo-1-oxo-spiro[3H-isoquinoline-4,1′-cyclopentane]-2-yl)acetic acid

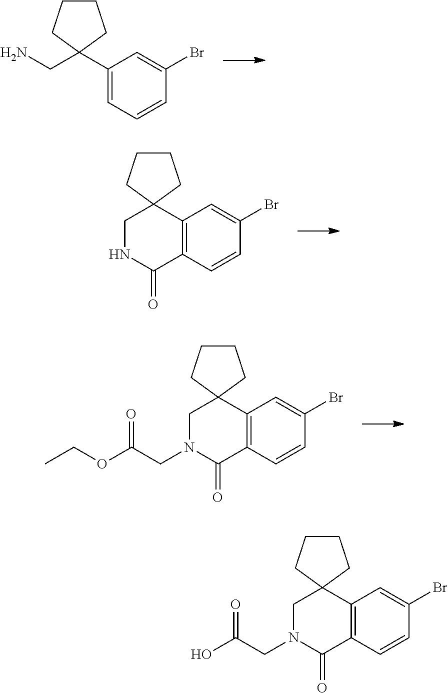

6-bromospiro[2,3-dihydroisoquinoline-4,1′-cyclopentane]-1-one: To a solution of [1-(3-bromophenyl)cyclopentyl]methanamine (300 mg, 1.18 mmol) in DCM (2.5 mL) were added a solution of triphosgene (140 mg, 0.47 mmol) in DCM (4 mL) followed by Et₃N (237 mg, 2.36 mmol). The reaction mixture was stirred for 2 h at 23° C. The reaction mixture was filtered through celite and the filtrate was added dropwise to a solution of aluminum chloride (644 mg, 4.72 mmol) in DCM (6 mL) at 0° C. This mixture was stirred at 0° C. for 45 min. The reaction mixture was diluted with aqueous HCl (30 mL, 0.1 N) and extracted with DCM (3×20 mL). The combined organics were washed with brine (3×20 mL), dried over anhydrous Na₂SO₄, filtered, and concentrated under reduced pressure. The crude residue was purified by reverse-phase HPLC. LCMS: m/z=280.0, 282.0 [M+H]⁺.

Ethyl 2-(6-bromo-1-oxospiro[3H-isoquinoline-4,1′-cyclopentane]-2-yl)acetate: To a solution of 6-bromospiro[2,3-dihydroisoquinoline-4,1′-cyclopentane]-1-one (59 mg, 0.21 mmol) in MeCN (1.5 mL) was added Cs₂CO₃ (138 mg, 0.42 mmol). The reaction mixture was stirred at 23° C. for 15 min followed by the addition of ethyl 2-iodoacetate (68 mg, 0.32 mmol). The reaction mixture was stirred at 55° C. for a further 2 h. The reaction mixture was cooled to 23° C., diluted with aqueous HCl (20 mL, 0.1 N), and extracted with EtOAc (4×20 mL). The combined organics were dried over anhydrous Na₂SO₄, filtered, and concentrated under reduced pressure to provide a residue that was used directly. LCMS: m/z=366.4, 368.3 [M+H]⁺.

2-(6-bromo-1-oxospiro[3H-isoquinoline-4,1′-cyclopentane]-2-yl)acetic acid: To a solution of ethyl 2-(6-bromo-1-oxo-spiro[3H-isoquinoline-4,1′-cyclopentane]-2-yl)acetate (88 mg, 0.24 mmol) in THF (1.5 mL) was added a solution of LiOH (12 mg, 0.48 mmol) in water (0.12 mL). The reaction mixture was stirred for 3 h at 23° C. The reaction mixture was adjusted pH=3-4 using aqueous HCl (1 N) and extracted with EtOAc (3×15 mL). The combined organic layers were washed with brine (20 mL), dried over anhydrous Na₂SO₄, filtered, and concentrated under reduced pressure to provide a residue that was used directly. LCMS: m/z=338.2, 340.2 [M+H]⁺.

### Intermediate 14

### 2-(6-bromo-4-methyl-1-oxo-3,4-dihydroisoquinolin-2-yl)acetic acid

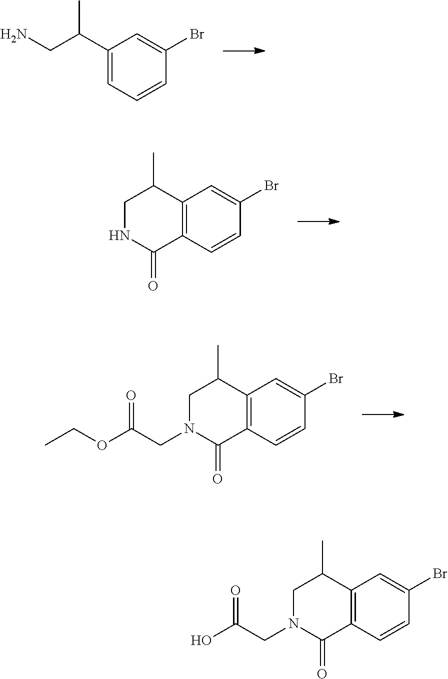 [SMILES: CC(CN)c1cccc(Br)c1 ; CC1CNC(=O)c2ccc(Br)cc21 ; CCOC(=O)CN1CC(C)c2cc(Br)ccc2C1=O ; CC1CN(CC(=O)O)C(=O)c2ccc(Br)cc21]

6-bromo-4-methyl-3,4-dihydro-2H-isoquinolin-1-one: To a solution triphosgene (140 mg, 0.47 mmol) in DCM (4 mL) was added 2-(3-bromophenyl)propan-1-amine (250 mg, 1.17 mmol) as a solution in DCM (2.5 mL) followed by Et₃N (237 mg, 2.36 mmol). The reaction mixture was stirred for 2 h at 23° C., and then filtered through celite. The resulting filtrate was added dropwise to a solution of AlCl₃ (637 mg, 4.67 mmol) in DCM (6 mL) at 0° C. The resultant mixture was stirred at 0° C. for 1 h. The reaction mixture was diluted with aqueous HCl (30 mL, 0.1 N) and extracted with DCM (3×20 mL). The combined organics were washed with brine (3×20 mL), dried over anhydrous Na₂SO₄, filtered, and concentrated under reduced pressure. The crude residue was purified by reverse-phase HPLC. LCMS: m/z=240.1, 242.0 [M+H]⁺.

ethyl 2-(6-bromo-4-methyl-1-oxo-3,4-dihydroisoquinolin-2-yl)acetate: To a solution of 6-bromo-4-methyl-3,4-dihydro-2H-isoquinolin-1-one (40 mg, 0.17 mmol) in MeCN (1.1 mL) was added Cs₂CO₃ (1.31 g, 0.33 mmol). The reaction mixture was stirred at 23° C. for 15 min followed by the addition of ethyl 2-iodoacetate (54 mg, 0.25 mmol). The reaction mixture was heated at 55° C. for a further 2 h. The reaction mixture was cooled to 23° C., diluted with aqueous HCl (20 mL, 0.1 N), and extracted with EtOAc (4×20 mL). The combined organics were dried over anhydrous Na₂SO₄, filtered, and concentrated under reduced pressure to provide a residue that was used directly. LCMS: m/z=326.2, 328.2 [M+H]⁺.

2-(6-bromo-4-methyl-1-oxo-3,4-dihydroisoquinolin-2-yl)acetic acid: To a solution of ethyl 2-(6-bromo-4-methyl-1-oxo-3,4-dihydroisoquinolin-2-yl)acetate (64 mg, 0.20 mmol) in THF (1.1 mL) was added a solution of LiOH (10 mg, 0.39 mmol) in water (0.10 mL). The reaction mixture was stirred for 3 h at 23° C. The reaction mixture was adjusted pH=3-4 using aqueous HCl (1 N) and extracted with EtOAc (3×15 mL). The combined organics were washed with brine (20 mL), dried over anhydrous Na₂SO₄, filtered, and concentrated under reduced pressure to provide a residue that was used directly. LCMS: m/z=298.1, 300.1 [M+H]⁺.

### Intermediate 15

### Methyl 2-(6-bromo-5-fluoro-1-oxo-spiro[3H-isoquinoline-4,1′-cyclopropane]-2-yl)acetate and methyl 2-(5-fluoro-1-oxo-spiro[3H-isoquinoline-4,1′-cyclopropane]-2-yl)acetate

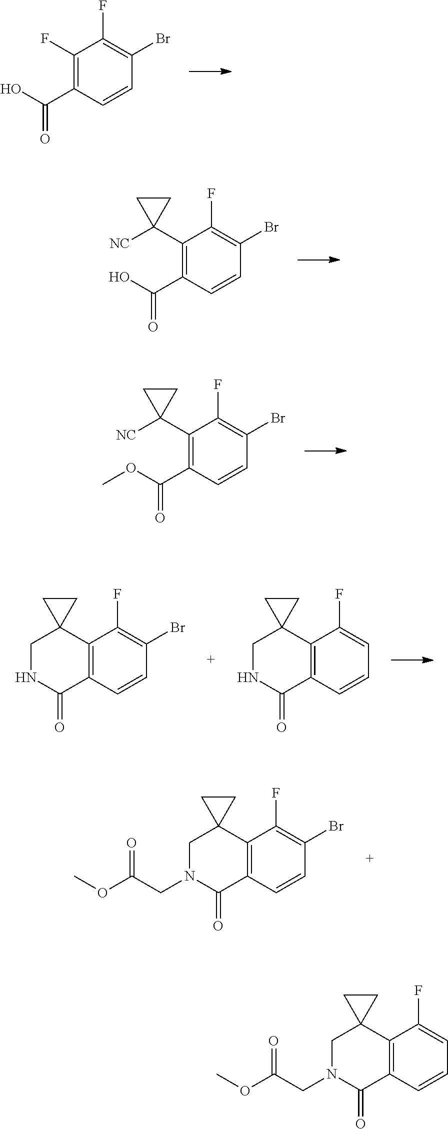

4-bromo-2-(1-cyanocyclopropyl)-3-fluorobenzoic acid: To a solution of 4-bromo-2,3-difluorobenzoic acid (4.50 g, 19.0 mmol) and cyclopropanecarbonitrile (3.80 g, 57.0 mmol) in THF (6.0 mL) at −40° C. was added KHMDS (49.4 mL, 1 M in THF). The reaction mixture was stirred at 20° C. for 1 h. The reaction mixture was diluted with water (10 mL) and extracted with EtOAc (3×10 mL). The aqueous was adjusted pH=3-4 using aqueous HCl (3.0 N) and then extracted with EtOAc (3×10 mL). The combined organics were washed with brine (20 mL), dried over anhydrous Na₂SO₄, filtered, and concentrated under reduced pressure to give a residue that was used directly. ¹H NMR (400 MHz, CD₃OD): δ 7.79-7.74 (m, 1H), 7.64-7.60 (m, 1H), 1.81-1.76 (m, 2H), 1.33-1.29 (m, 2H).

Methyl 4-bromo-2-(1-cyanocyclopropyl)-3-fluorobenzoate: To a solution of 4-bromo-2-(1-cyanocyclopropyl)-3-fluorobenzoic acid (4.80 g, 16.9 mmol) in THF (50 mL) at 0° C. was added TMSCHN₂ (16.9 mL, 2 M in n-hexane). The reaction mixture was stirred at 20° C. for 16 h. The reaction mixture was diluted with glacial acetic acid (20 mL) stirred at 20° C. for a further 30 min. The reaction mixture was diluted with water (20 mL) and extracted with EtOAc (3×10 mL). The combined organics were washed with brine (15 mL), dried over anhydrous Na₂SO₄, filtered, and concentrated under reduced pressure. The residue was purified by silica gel column chromatography. ¹H NMR (400 MHz, CD₃OD): δ 7.82-7.76 (m, 1H), 7.63-7.57 (m, 1H), 3.98 (s, 3H), 1.81-1.75 (m, 2H), 1.31-1.25 (m, 2H).

6-bromo-5-fluoro-spiro[2,3-dihydroisoquinoline-4,1′-cyclopropane]-1-one and 5-fluoro-spiro[2,3-dihydroisoquinoline-4,1′-cyclopropane]-1-one: To a solution of methyl 4-bromo-2-(1-cyanocyclopropyl)-3-fluorobenzoate (1.8 g, 6.04 mmol) and dichlorocobalt (1.57 g, 12.1 mmol) in MeOH (3.0 mL) at 0° C. was added NaBH₄ (1.15 g, 30.4 mmol). The reaction mixture was stirred at 20° C. for 16 h. The reaction mixture was diluted with sat. aq. NH₄Cl (10 mL) and water (5 mL) and extracted with EtOAc (3×8 mL). The combined organics were washed with brine, dried over anhydrous Na₂SO₄, filtered, and concentrated under reduced pressure to give a residue as a 3:1 mixture in favor of 5-fluoro-spiro[2,3-dihydroisoquinoline-4,1-cyclopropane]-1-one that was used directly: 6-bromo-5-fluoro-spiro[2,3-dihydroisoquinoline-4,1′-cyclopropane]-1-one. LCMS: m/z=270.0, 272.0 [M+H]⁺; 5-fluoro-spiro[2,3-dihydroisoquinoline-4,1′-cyclopropane]-1-one: LCMS: m/z=192.1 [M+H]⁺.

Methyl 2-(6-bromo-5-fluoro-1-oxo-spiro[3H-isoquinoline-4,1′-cyclopropane]-2-yl)acetate and methyl 2-(5-fluoro-1-oxo-spiro[3H-isoquinoline-4,1′-cyclopropane]-2-yl)acetate: To a mixture of 6-bromo-5-fluoro-spiro[2,3-dihydroisoquinoline-4,1′-cyclopropane]-1-one and 5-fluoro-spiro[2,3-dihydroisoquinoline-4,1′-cyclopropane]-1-one (500 mg, 1.85 mmol, 1:3 ratio) in DMF (3.0 mL) at 0° C. was added NaH (74 mg, 1.85 mmol, 60% purity). The reaction mixture was stirred at 0° C. for 30 min followed by the addition of methyl 2-bromoacetate (425 mg, 2.78 mmol). The reaction mixture was stirred at 20° C. for a further 1.5 h. The reaction mixture was diluted with water (5 mL) and then extracted with EtOAc (3×5 mL). The combined organics were washed with brine (5 mL), dried over anhydrous Na₂SO₄, filtered, and concentrated under reduced pressure. The residue was purified by silica gel column chromatography to provide a 3:1 mixture of the title compounds in favor of methyl 2-(5-fluoro-1-oxo-spiro[3H-isoquinoline-4,1′-cyclopropane]-2-yl)acetate:

methyl 2-(6-bromo-5-fluoro-1-oxo-spiro[3H-isoquinoline-4,1′-cyclopropane]-2-yl)acetate: LCMS: m/z=342.0, 344.0 [M+H]⁺; methyl 2-(5-fluoro-1-oxo-spiro[3H-isoquinoline-4,1′-cyclopropane]-2-yl)acetate: LCMS: m/z=264.1 [M+H]+

### Intermediate 16

### methyl 2-(6-cyano-1-oxo-spiro[3H-isoquinoline-4,1′-cyclopropane]-2-yl)acetate

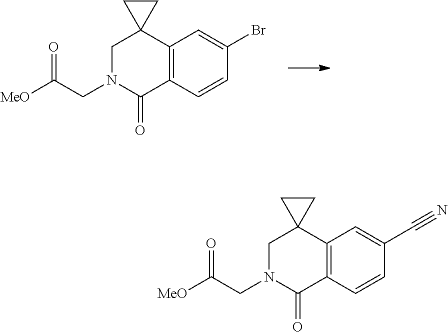 [SMILES: COC(=O)CN1CC2(CC2)c2cc(Br)ccc2C1=O ; COC(=O)CN1CC2(CC2)c2cc(C#N)ccc2C1=O]

To a solution of methyl 2-(6-bromo-1-oxo-spiro[3H-isoquinoline-4,1′-cyclopropane]-2-yl)acetate (100 mg, 0.31 mmol) in DMF (3.1 mL) were added zinc cyanide (54 mg, 0.46 mmol) and Pd(PPh₃)₄ (71 mg, 0.2 mmol). The suspension was sparged with N₂ and stirred at 100° C. for 3 h. The reaction mixture was cooled to ambient temperature, diluted with water (10 mL), and extracted with EtOAc (3×10 mL). The combined organic layers were washed with brine, dried over anhydrous Na₂SO₄, filtered, and concentrated under reduced pressure. The residue was purified by silica gel column chromatography. LCMS: m/z=271.2 [M+H]⁺. ¹H NMR (400 MHz, CDCl₃): δ 8.19 (dd, J=8.0, 0.5 Hz, 1H), 7.57 (dd, J=8.0, 1.5 Hz, 1H), 7.14 (dd, J=1.5, 0.4 Hz, 1H), 4.34 (s, 2H), 3.75 (s, 3H), 3.48 (s, 2H), 1.16-1.09 (m, 4H).

### Intermediate 17

### 2-(6-fluoro-1-oxo-spiro[3H-isoquinoline-4,1′-cyclopropane]-2-yl)acetic acid

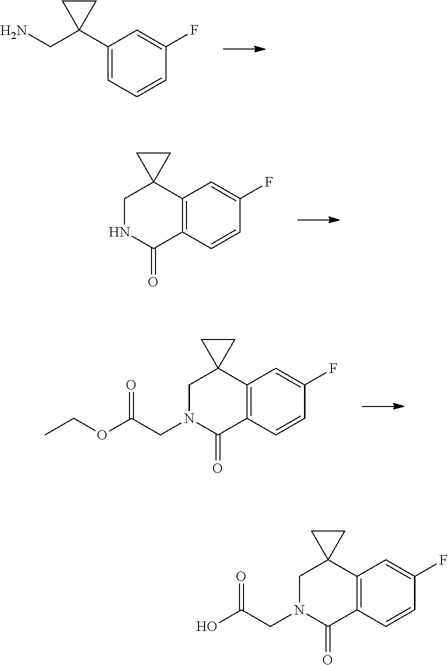 [SMILES: NCC1(c2cccc(F)c2)CC1 ; O=C1NCC2(CC2)c2cc(F)ccc21 ; CCOC(=O)CN1CC2(CC2)c2cc(F)ccc2C1=O ; O=C(O)CN1CC2(CC2)c2cc(F)ccc2C1=O]

6-fluorospiro[2,3-dihydroisoquinoline-4,1′-cyclopropane]-1-one: To a solution of [1-(3-fluorophenyl)cyclopropyl]methanamine (300 mg, 1.82 mmol) in DCM (3 mL) was added a solution of triphosgene (215 mg, 0.73 mmol) in DCM (3 mL) followed by Et₃N (367 mg, 3.63 mmol). The reaction mixture was stirred for 2 h at 23° C. The reaction mixture was filtered through celite, and the filtrate was added dropwise to a solution of aluminum chloride (990 mg, 7.26 mmol) in DCM (6 mL) at 0° C. This mixture was stirred at 0° C. for 60 min. The reaction mixture was diluted with aqueous HCl (50 mL, 0.1 N) and extracted with DCM (3×20 mL). The combined organics were washed with brine (3×20 mL), dried over anhydrous Na₂SO₄, filtered, and concentrated under reduced pressure. The crude residue was purified by reverse-phase preparative HPLC. LCMS: m/z=192.1 [M+H]⁺.

Ethyl 2-(6-fluoro-1-oxo-spiro[3H-isoquinoline-4,1′-cyclopropane]-2-yl)acetate: To a solution of 6-fluorospiro[2,3-dihydroisoquinoline-4,1′-cyclopropane]-1-one (32 mg, 0.17 mmol) in MeCN (1.2 mL) was added Cs₂CO₃ (110 mg, 0.33 mmol). The reaction mixture was stirred at 23° C. for 15 min followed by the addition of ethyl 2-iodoacetate (54 mg, 0.25 mmol). The reaction mixture was stirred at 55° C. for 24 h. The reaction mixture was cooled to 23° C., diluted with aq. HCl (15 mL, 0.1 N), and extracted with EtOAc (4×15 mL). The combined organics were dried over anhydrous Na₂SO₄, filtered, and concentrated under reduced pressure to provide a residue that was used directly. LCMS: m/z=278.1 [M+H]⁺.

2-(6-fluoro-1-oxo-spiro[3H-isoquinoline-4,1′-cyclopropane]-2-yl)acetic acid: To a solution of ethyl 2-(6-fluoro-1-oxo-spiro[3H-isoquinoline-4,1′-cyclopropane]-2-yl)acetate (45 mg, 0.16 mmol) in THF (1.0 mL) was added a solution of LiOH (12 mg, 0.48 mmol) in water (0.12 mL). The reaction mixture was stirred for 3 h at 23° C. The reaction mixture was adjusted to pH=3-4 using aq. HCl (1 N) and extracted with EtOAc (3×20 mL). The combined organic layers were washed with brine (20 mL), dried over anhydrous Na₂SO₄, filtered, and concentrated under reduced pressure to provide a residue that was used directly. LCMS: m/z=250.1 [M+H]⁺.

### Intermediate 18

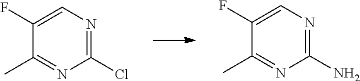

5-fluoro-4-methylpyrimidin-2-amine: To a solution of 2-chloro-5-fluoro-4-methylpyrimidine (300 mg, 2.05 mmol) in i-PrOH (1 mL) was added NH₃—H₂O (0.63 mL) at 25° C. Then the mixture was stirred at 100° C. for 1.5 h. The reaction mixture was filtered and the filter cake was concentrated under reduced pressure. ¹H NMR (400 MHz, DMSO-d₆): δ 8.11 (d, J=1.6 Hz, 1H), 6.48 (br s, 2H), 2.23 (d, J=2.4 Hz, 3H).

### Intermediate 19

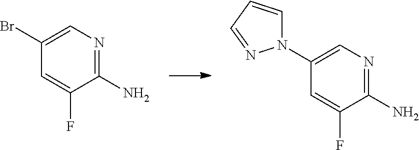 [SMILES: Nc1ncc(Br)cc1F ; Nc1ncc(-n2cccn2)cc1F]

3-fluoro-5-(1H-pyrazol-1-yl)pyridin-2-amine: To a solution of 5-bromo-3-fluoro-pyridin-2-amine (500 mg, 2.62 mmol) and 1H-pyrazole (149 mg, 2.18 mmol) in DMF (5 mL) was added (1R,2R)—N¹,N²-dimethylcyclohexane-1,2-diamine (62.1 mg, 0.44 mmol), CuI (41.6 mg, 0.22 mmol) and K₂CO₃ (452 mg, 3.27 mmol) under N₂. The reaction mixture was stirred at 120° C. for 12 h. The reaction mixture was quenched by addition of H₂O (30 mL) and extracted with EtOAc (5×30 mL). The combined organics were washed with brine (30 mL), dried over anhydrous Na₂SO₄, filtered, and concentrated under reduced pressure. The residue was purified by silica gel column chromatography. ¹H NMR (400 MHz, CDCl₃): δ 8.19 (d, J=2.0 Hz, 1H), 7.80 (d, J=2.4 Hz, 1H), 7.71-7.67 (m. 2H), 6.47 (s, 1H), 4.73 (br s, 2H).

### Intermediate 20

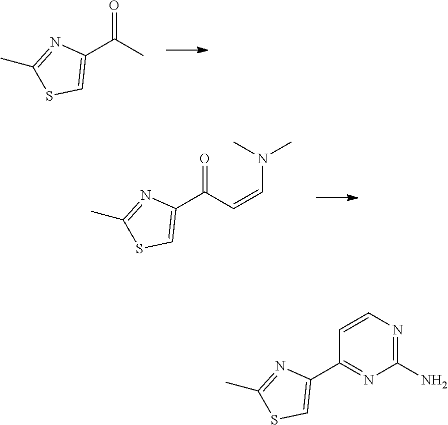

(Z)-3-(dimethylamino)-1-(2-methylthiazol-4-yl)prop-2-en-1-one: A solution of 1-(2-methylthiazol-4-yl)ethanone (3.0 g, 21.3 mmol) in N,N-Dimethylformamide dimethyl acetal (10 mL) was heated at 90° C. in a sealed tube for 16 h. The reaction mixture was cooled to 15° C., filtered, and the filter cake was dried under reduced pressure to provide a solid that was used directly. LCMS: m/z=197.1 [M+H]⁺.

4-(2-methylthiazol-4-yl)pyrimidin-2-amine: To a solution of (Z)-3-(dimethylamino)-1-(2-methylthiazol-4-yl)prop-2-en-1-one (1.0 g, 5.10 mmol) and guanidine hydrochloride (487 mg, 5.10 mmol) in EtOH (20 mL) was added NaOH (245 mg, 6.11 mmol). The reaction mixture was stirred at 80° C. for 32 h. The reaction mixture was concentrated under reduced pressure, and the resulting residue was suspended in H₂O (10 mL). This solution was filtered and the filter cake was dried under reduced pressure to provide a solid that was used directly. LCMS: m/z=193.1 [M+H]⁺.

### Intermediate 21

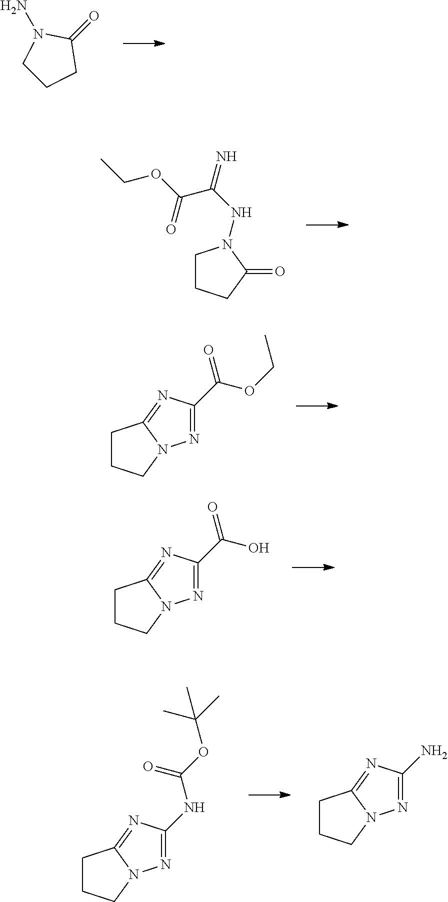 [SMILES: NN1CCCC1=O ; CCOC(=O)C(=N)NN1CCCC1=O ; CCOC(=O)c1nc2n(n1)CCC2 ; O=C(O)c1nc2n(n1)CCC2 ; CC(C)(C)OC(=O)Nc1nc2n(n1)CCC2 ; Nc1nc2n(n1)CCC2]

Ethyl 2-imino-2-((2-oxopyrrolidin-1-yl)amino)acetate: To a solution of 1-aminopyrrolidin-2-one HCl salt (10 g, 73.2 mmol) and ethyl 2-ethoxy-2-imino-acetate (15.9 g, 110 mmol) in EtOH (120 mL) was added Et₃N (7.41 g, 73.2 mmol). The reaction mixture was stirred at 60° C. for 5 h. The reaction mixture was concentrated under reduced pressure to give a residue that was used directly. LCMS: m/z=200.1 [M+H]⁺.

Ethyl 6,7-dihydro-5H-pyrrolo[1,2-b][1,2,4]triazole-2-carboxylate: A solution of ethyl 2-imino-2-((2-oxopyrrolidin-1-yl)amino)acetate (15.0 g, 75.3 mmol) in POCl₃ (173 g, 1.13 mol) was stirred at 120° C. for 3 h. The reaction mixture was concentrated under reduced pressure, and the resulting solution was added dropwise to sat. aq. NaHCO₃ (240 mL) and extracted with EtOAc (3×80 mL). The combined organics were washed with brine (80 mL), dried over anhydrous Na₂SO₄, filtered, and concentrated under reduced pressure. The residue was purified by silica gel column chromatography. LCMS: m/z=182.1 [M+H]⁺.

6,7-dihydro-5H-pyrrolo[1,2-b][1,2,4]triazole-2-carboxylic acid: To a solution of ethyl 6,7-dihydro-5H-pyrrolo[1,2-b][1,2,4]triazole-2-carboxylate (3.0 g, 16.6 mmol) in 1,4-dioxane (15 mL) was added aq. HCl (30.4 mL, 3 M). The reaction mixture was stirred at 100° C. for 16 h. The reaction mixture was concentrated under reduced pressure, and the resulting residue was slurried with MTBE:DCM (3:1, 20 mL) at 25° C. to give a solid that was filtered and used directly. LCMS: m/z=154.1 [M+H]⁺.

tert-butyl N-(6,7-dihydro-5H-pyrrolo[1,2-b][1,2,4]triazol-2-yl)carbamate: To a solution of 6,7-dihydro-5H-pyrrolo[1,2-b][1,2,4]triazole-2-carboxylic acid (2.6 g, 16.9 mmol) in toluene (26 mL) was added Et₃N (2.6 g, 25.5 mmol) and DPPA (5.6 g, 20.4 mmol). The reaction mixture was stirred at 25° C. for 16 h, followed by addition of t-BuOH (20.1 g, 272 mmol). The reaction mixture was stirred at 80° C. for an additional 4 h. The reaction mixture was cooled to ambient temperature, quenched with water (120 mL), and extracted with EtOAc (3×40 mL). The combined organics were washed with brine (40 mL), dried over anhydrous Na₂SO₄, filtered, and concentrated under reduced pressure. The residue was purified by silica gel column chromatography. The resulting residue was slurried with MTBE (30 mL) and the filter cake was collected and dried under reduced pressure. LCMS: m/z=225.1 [M+H]⁺.

6,7-dihydro-5H-pyrrolo[1,2-b][1,2,4]triazol-2-amine: A solution of tert-butyl N-(6,7-dihydro-5H-pyrrolo[1,2-b][1,2,4]triazol-2-yl)carbamate (500 mg, 2.23 mmol) in HCl (10 mL, 4 M in EtOAc) was stirred at 20° C. for 4 h. The reaction mixture was concentrated under reduced pressure to give a solid that was used directly. ¹H NMR (400 MHz, DMSO-d₆) δ 4.05 (t, J=7.2 Hz, 2H). 3.02 (t, J=7.2 Hz, 2H), 2.60 (m, 2H).

### Intermediate 22

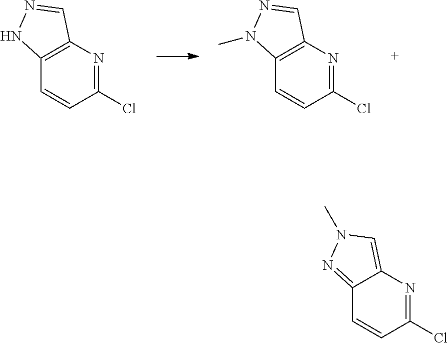

5-chloro-1-methyl-1H-pyrazolo[4,3-b]pyridine: To a solution of 5-chloro-1H-pyrazolo[4,3-b]pyridine (2.0 g, 13.0 mmol) in acetone (20 mL) at 0° C. was added KOH (2.19 g, 39.1 mmol). The reaction mixture was stirred for 1 h at 0° C. followed by the addition of Mel (1.22 mL, 19.5 mmol). The reaction mixture was stirred at 25° C. for another 12 h. The reaction mixture was poured into H₂O (40 mL) and extracted with EtOAc (3×40 mL). The combined organic layers were washed with brine (40 mL), dried over anhydrous Na₂SO₄, filtered, and concentrated under reduced pressure. The residue was purified by silica gel column chromatography.

### Intermediate 23

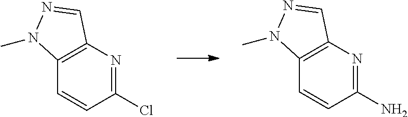

1-methyl-1H-pyrazolo[4,3-b]pyridin-5-amine: To a solution of 5-chloro-1-methyl-1H-pyrazolo[4,3-b]pyridine (1.0 g, 5.97 mmol) in THF (10 mL) was added diphenylmethanimine (1.30 g, 7.16 mmol), Pd₂(dba)₃ (1.09 g, 1.19 mmol), X-phos (1.14 g, 2.39 mmol) and LiHMDS (1 M in THF, 7.16 mL). The reaction mixture was stirred at 65° C. for 10 h. To the reaction mixture was added 2N HCl (30 mL) and THF (10 mL), and the reaction mixture was stirred at 25° C. for 30 min. The reaction mixture was adjusted to pH=9 by the addition of solid Na₂CO₃. The reaction mixture was poured into H₂O (20 mL) and extracted with EtOAc (3×20 mL). The combined organics were washed with brine (20 mL), dried over anhydrous Na₂SO₄, filtered, and concentrated under reduced pressure. The residue was purified by silica gel column chromatography. LCMS: m/z=149.1 [M+H]⁺.

### Intermediate 24

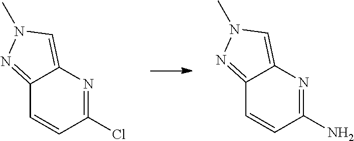

2-methyl-2H-pyrazolo[4,3-b]pyridin-5-amine: To a solution of 5-chloro-2-methyl-2H-pyrazolo[4,3-b]pyridine (1.0 g, 5.97 mmol) and diphenylmethanimine (1.3 g, 7.16 mmol) in THF (10 mL) at 0° C. was added X-Phos (1.1 g, 2.39 mmol), Pd₂(dba)₃ (1.1 g, 1.19 mmol) and LiHMDS (1 M in THF, 7.16 mL). The reaction mixture was stirred at 65° C. for 10 h. To the reaction mixture was added aq. HCl (30 mL, 2 N) and THF (10 mL), and the reaction mixture was stirred at 25° C. for 30 min. The reaction mixture was adjusted to pH=9 by the addition of solid Na₂CO₃. The reaction mixture was diluted with water (20 mL) and extracted with EtOAc (3×20 mL). The combined organics were washed with brine (20 mL), dried over anhydrous Na₂SO₄, filtered, and concentrated under reduced pressure. The residue was purified by silica gel column chromatography. ¹H NMR (400 MHz, DMSO-d₆) δ 7.84 (d, J=1.2 Hz, 1H), 7.67-7.65 (m, 1H), 6.58-6.55 (m, 1H). 5.91 (br s, 2H), 4.01 (d, J=2.0 Hz, 3H).

### Intermediate 25

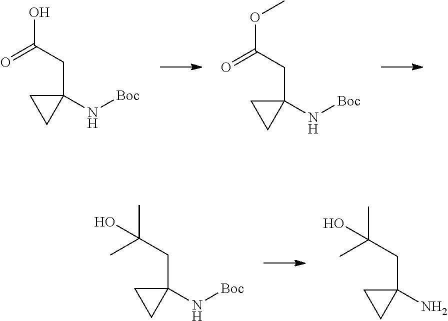

Methyl 2-(1-((tert-butoxycarbonyl)amino)cyclopropyl)acetate: To a solution of 2-(1-((tert-butoxycarbonyl)amino)cyclopropyl)acetic acid (300 mg, 1.39 mmol) in THF (3 mL) and MeOH (1 mL) was added TMSCHN₂ (2 M in hexanes, 1.39 mL) at 0° C., the mixture was stirred at 25° C. for 2 h. The mixture was quenched by addition of Na₂S₂O₃ (10 mL) and extracted with EtOAc (3×6 mL). The combined organic layers were washed with brine (10 mL), dried over anhydrous Na₂SO₄, filtered and concentrated under reduced pressure to give a residue. The mixture was purified by preparatory TLC.

tert-butyl (1-(2-hydroxy-2-methylpropyl)cyclopropyl)carbamate: To a solution of methyl 2-(1-((tert-butoxycarbonyl)amino)cyclopropyl)acetate (100 mg, 0.44 mmol) in THF (2 mL) was added MeMgBr (3 M in ether, 0.58 mL) at −78° C., and then the mixture was stirred at 25° C. for 1 h. The mixture was poured into ice water (10 mL) and extracted with EtOAc (3×8 mL). The combined organic layers were washed with brine (15 mL), dried over anhydrous Na₂SO₄, filtered and concentrated under reduced pressure to provide a residue that was used directly.

1-(1-aminocyclopropyl)-2-methylpropan-2-ol: A solution of tert-butyl (1-(2-hydroxy-2-methylpropyl)cyclopropyl)carbamate (40 mg, 0.17 mmol) in 4 M HCl/MeOH (0.5 mL) was stirred at 25° C. for 0.5 h. The mixture was concentrated under reduced pressure. Crude material was used in the next step without purification.

### Intermediate 26

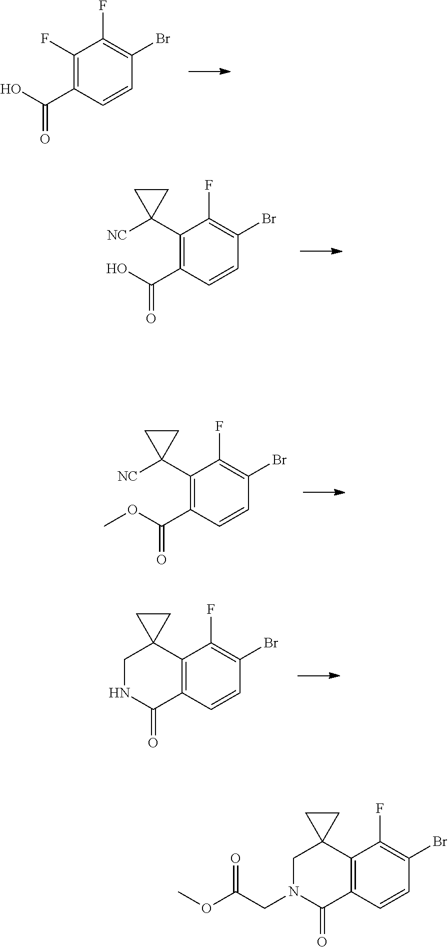 [SMILES: O=C(O)c1ccc(Br)c(F)c1F ; N#CC1(c2c(C(=O)O)ccc(Br)c2F)CC1 ; COC(=O)c1ccc(Br)c(F)c1C1(C#N)CC1 ; O=C1NCC2(CC2)c2c1ccc(Br)c2F ; COC(=O)CN1CC2(CC2)c2c(ccc(Br)c2F)C1=O]

4-bromo-2-(1-cyanocyclopropyl)-3-fluorobenzoic acid: To a solution of 4-bromo-2,3-difluorobenzoic acid (40 g, 169 mmol) in THF (700 mL) was added cyclopropanecarbonitrile (34 g, 506 mmol). To the reaction mixture was cooled to −40° C. followed by the addition of KHMDS (438.8 mL, 1M in THF). The reaction mixture was stirred at 20° C. for 1 h, diluted with water (500 mL), and extracted with EtOAc (2×100 mL). The aqueous phase was adjusted to pH=3 with aq. HCl (3 N) and extracted with EtOAc (3×200 mL). The combined organics were dried over anhydrous Na₂SO₄, filtered, and concentrated under reduced pressure to provide a solid that was used directly. LCMS: m/z=284.0, 285.9 [M+H]⁺.

Methyl 4-bromo-2-(1-cyanocyclopropyl)-3-fluorobenzoate: To a solution of 4-bromo-2-(1-cyanocyclopropyl)-3-fluorobenzoic acid (46 g, 162 mmol) in DMF (500 mL) at 0° C. was added K₂CO₃ (34 g, 243 mmol). To the mixture was added Mel (23 g, 162 mmol). The reaction mixture was stirred at 20° C. for 2 h. The reaction mixture was poured into ice cold water (500 mL), filtered, and the filter cake was washed with petroleum ether (2×100 mL). The filtrate was concentrated under reduced pressure to provide a solid that was used directly. LCMS: m/z=298.1, 300.0 [M+H]⁺.

6-bromo-5-fluoro-spiro[2,3-dihydroisoquinoline-4,1′-cyclopropane]-1-one: To a solution of methyl 4-bromo-2-(1-cyanocyclopropyl)-3-fluorobenzoate (42 g, 141 mmol) in MeOH (600 mL) and water (20 mL) at 0° C. was added dichlorocobalt (73 g, 563 mmol) and NaBH₄ (27.0 g, 704 mmol). The reaction mixture was stirred at 20° C. for 4 h. The reaction mixture was poured into sat. aq. NH₄Cl (600 mL), diluted with water (300 mL), and extracted with EtOAc (3×200 mL). The combined organics were dried over anhydrous Na₂SO₄, filtered, and concentrated under reduced pressure. The residue was purified by silica gel chromatography. LCMS: m/z=270.0, 272.0 [M+H]⁺.

Methyl 2-(6-bromo-5-fluoro-1-oxospiro[3H-isoquinoline-4,1′-cyclopropane]-2-yl)acetate: To a solution of 6-bromo-5-fluoro-spiro[2,3-dihydroisoquinoline-4,1′-cyclopropane]-1-one (9.3 g, 34.4 mmol) in DMF (100 mL) at 0° C. was added NaH (2.1 g, 51.6 mmol, 60% purity). The reaction mixture was stirred at 0° C. for 0.5 h. To the reaction mixture was added methyl 2-bromoacetate (11 g, 68.9 mmol, 6.50 mL) at 0° C. The reaction mixture was stirred at 20° C. for 16 h. The reaction mixture was quenched by the addition sat. aq. NH₄Cl(50 mL) and ice-cold water (300 mL) and extracted with EtOAc (3×80 mL). The combined organics were washed with brine (200 mL), dried over anhydrous Na₂SO₄, filtered, and concentrated under reduced pressure. The residue was purified by silica gel chromatography, and the resulting product was slurried with MTBE:PE (2:1, 5 mL) at 25° C. to give a solid that was filtered and dried under reduced pressure. LCMS: m/z=342.0, 344.0 [M+H]⁺. ¹H NMR (400 MHz, CDCl₃): δ 7.84 (dd, J=0.8, 8.4 Hz. 1H), 7.52-7.46 (m, 1H), 4.32 (s, 2H), 3.77 (s, 3H), 3.38 (s, 2H), 1.66-1.60 (m, 2H), 1.04-0.99 (m, 2H).

### Intermediate 27

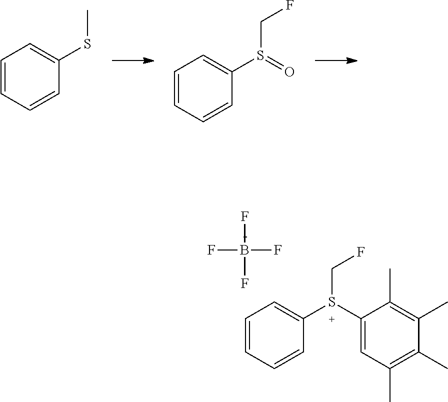

((fluoromethyl)sulfinyl)benzene: To a suspension of SelectFluor (463.5 g, 1.31 mol) in MeCN (1500 mL) at 0° C. was added a solution of methyl(phenyl)sulfane (130.0 g, 1.05 mol) in MeCN (150 mL) over 10 min. Et₃N (132.4 g, 1.31 mol) was then added to the mixture at 0° C. The reaction mixture was allowed to warm to 20° C., and stirred for 16 h. Two batches of the above were run in parallel and combined for workup. The combined batches were diluted with water (1000 mL) and extracted with DCM (3×500 mL). The combined organic layers were dried over anhydrous Na₂SO₄, filtered, and concentrated under reduced pressure. The crude residue was then dissolved in MeOH (2000 mL), diluted with water (200 mL), and cooled to 0° C. NBS (372.6 g, 2.09 mol) was carefully added and the mixture was stirred for 16 h at 15° C. The reaction mixture was diluted with 10% Na₂SO₃ solution (200 mL) followed by sat. aq. NaHCO₃ until pH=7. Methanol was removed under reduced pressure and the remaining aqueous phase was extracted with DCM (3×500 mL). The combined organic layers were washed with brine (500 mL), dried over anhydrous Na₂SO₄, filtered, and concentrated under reduced pressure

(fluoromethyl)(phenyl)(2,3,4,5-tetramethylphenyl)sulfonium tetrafluoroborate: To a solution of ((fluoromethyl)sulfinyl)benzene (10.0 g, 63.2 mmol) in diisopropyl ether (100 mL) at −10° C. were added 1,2,3,4-tetramethylbenzene (7.64 g, 56.9 mmol) and Tf₂O (17.8 g, 63.2 mmol). The reaction mixture was allowed to warm to 20° C., and stirred for 1 h. The reaction mixture was filtered and the resulting solid was dissolved in DCM (200 mL) and washed with aq. NaBF₄ (1 M, 6×200 mL). The organic phase was dried over anhydrous Na₂SO₄, filtered, and concentrated under reduced pressure. The residue was triturated with MTBE at 20° C. for 30 min and then filtered to provide the desired product. LCMS: m/z=275.2 [M+H]⁺.

### Intermediate 28

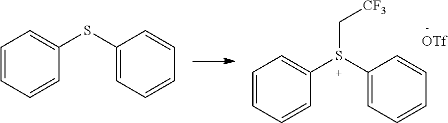

Diphenyl(2,2,2-trifluoroethyl)sulfonium trifluoromethanesulfonate: A mixture of diphenylsulfane (36.1 g, 193.9 mmol) and 2,2,2-trifluoroethyl trifluoromethanesulfonate (9 g, 38.8 mmol) was stirred at 150° C. for 20 h in a 100 mL sealed tube. The mixture was cooled to 20° C., and added in MTBE (200 mL). The resulting mixture was filtered and the solid was concentrated under reduced pressure. ¹H NMR (400 MHz, Acetone-d₆): δ 8.38-8.36 (m, 4H), 7.96-7.92 (m, 2H), 7.87-7.83 (m, 4H), 5.79-5.72 (m, 2H).

### Intermediates 29 and 30

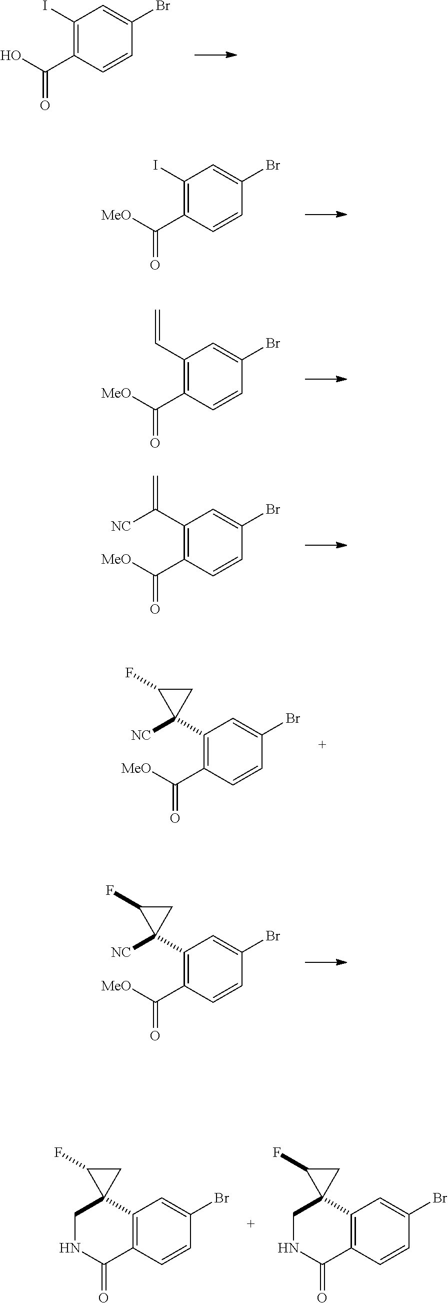

Methyl 4-bromo-2-iodobenzoate: To a solution of 4-bromo-2-iodo-benzoic acid (10.0 g, 30.6 mmol) in MeOH (100 mL) at 0° C. was added conc. H₂SO₄ (15 g, 153 mmol). The reaction mixture was stirred at 80° C. for 5 h. The reaction mixture was poured into water (300 mL) and extracted with EtOAc (3×100 mL). The combined organic layers were washed with sat. aq. NaHCO₃ (3×50 mL), dried over anhydrous Na₂SO₄, filtered, and concentrated under reduced pressure to provide a crude residue that was used directly. ¹H NMR (400 MHz, CDCl₃): δ 8.18 (s, 1H), 7.70 (d, J=8.4 Hz, 1H), 7.55 (dd, J=0.8, 8.0 Hz, 1H), 3.93 (s, 3H).

Methyl 4-bromo-2-vinylbenzoate: To a mixture of methyl 4-bromo-2-iodo-benzoate (6.0 g, 17.6 mmol), potassium vinyltrifluoroborate (2.4 g, 17.6 mmol), and CsF (8.0 g, 52.8 mmol) in 1,4-dioxane (100 mL) was added Pd(dppf)Cl₂ (1.3 g, 1.76 mmol). The reaction mixture was stirred at 90° C. for 16 h. The reaction mixture was concentrated under reduced pressure. The residue was purified by silica gel column chromatography. ¹H NMR (400 MHz, CDCl₃): δ 7.77 (d, J=8.4 Hz, 1H), 7.72 (d, J=2.0 Hz, 1H), 7.49-7.37 (m, 2H), 5.66 (d, J=17.2 Hz, 1H), 5.41 (d, J=10.8 Hz, 1H), 3.90 (s, 3H).

Methyl 4-bromo-2-(1-cyanovinyl)benzoate: To a mixture of Cu₂O (345 mg, 2.41 mmol), 6,6′-dimethyl-2,2′-bipyridine (444 mg, 2.41 mmol), and Selectfluor (6.4 g, 18 mmol) in acetone (20 mL) and water (10 mL) were added methyl 4-bromo-2-vinyl-benzoate (2.9 g, 12 mmol) and TMSCN (2.4 g, 24.1 mmol). The reaction mixture was stirred at 20° C. for 16 h. The reaction mixture was poured into aq. NaHCO₃ (1 M, 30 mL) and extracted with EtOAc (3×30 mL). The combined organic layers were washed with brine (30 mL), dried over anhydrous Na₂SO₄, filtered, and concentrated under reduced pressure. The residue was purified by silica gel column chromatography. ¹H NMR (400 MHz, CDCl₃): δ 7.89 (d, J=8.4 Hz, 1H), 7.65 (dd, J=2.0, 8.4 Hz, 1H), 7.51 (d, J=1.6 Hz, 1H), 6.24 (s, 1H), 6.01 (s, 1H), 3.96 (s, 3H).

Methyl 4-bromo-2-((1r,2r)-1-cyano-2-fluorocyclopropyl)benzoate and methyl 4-bromo-2-((1r,2s)-1-cyano-2-fluorocyclopropyl)benzoate: To a solution of methyl 4-bromo-2-(1-cyanovinyl)benzoate (800 mg, 3.01 mmol) in THF (30 mL) at 0° C. were added (fluoromethyl)(phenyl)(2,3,4,5-tetramethylphenyl)sulfonium tetrafluoroborate (2.3 g, 6.0 mmol) and NaH (1.2 g, 30.1 mmol, 60% purity in mineral oil). The reaction mixture was stirred at 20° C. for 1 h. The reaction mixture was poured into water (50 mL) and extracted with EtOAc (3×30 mL). The combined organic layers were washed with brine (10 mL), dried over anhydrous Na₂SO₄, filtered, and concentrated under reduced pressure. The residue was purified by silica gel column chromatography to provide the title products as a 3:2 mixture. LCMS: m/z=298.0, 300.0 [M+H]⁺.

(2′s,4r)-6-bromo-2′-fluorospiro[2,3-dihydroisoquinoline-4,1′-cyclopropane]-1-one and (2′r,4r)-6-bromo-2′-fluorospiro[2,3-dihydroisoquinoline-4,1′-cyclopropane]-1-one: To a solution of methyl 4-bromo-2-((1r,2r)-1-cyano-2-fluorocyclopropyl)benzoate and methyl 4-bromo-2-((1r,2s)-1-cyano-2-fluorocyclopropyl)benzoate (0.92 g, 3.09 mmol, 3:2 mixture) and dichlorocobalt (401 mg, 3.09 mmol) in MeOH (10 mL) at 0° C. was added NaBH₄ (350 mg, 9.26 mmol). The reaction mixture was stirred at 20° C. for 2 h. The reaction mixture was poured into sat. aq. NH₄Cl (20 mL) and extracted with EtOAc (3×20 mL). The combined organic layers were washed with brine (2×10 mL), dried over anhydrous Na₂SO₄, filtered, and concentrated under reduced pressure. The residue was purified by silica gel column chromatography to provide:

(2′s,4r)-6-bromo-2′-fluorospiro[2,3-dihydroisoquinoline-4,1′-cyclopropane]-1-one (Int. 29): ¹H NMR (400 MHz, CDCl₃): δ 8.02 (d, J=8.4 Hz, 1H), 7.52 (dd, J=2.0, 8.4 Hz, 1H), 6.85 (d, J=2.0 Hz, 1H), 6.12 (br s, 1H). 4.70-4.50 (m, 1H), 3.87 (d, J=12.4 Hz, 1H), 3.51 (dd, J=4.4, 12.8 Hz, 1H), 1.62-1.54 (m, 1H), 1.42-1.24 (m, 1H).

(2′r,4r)-6-bromo-2′-fluorospiro[2,3-dihydroisoquinoline-4,1′-cyclopropane]-1-one (Int. 30): ¹H NMR (400 MHz, CDCl₃): δ 8.02 (d, J=8.4 Hz, 1H), 7.55 (dd, J=2.0, 8.4 Hz, 1H), 7.28 (d, J=2.0 Hz, 1H), 6.56 (br s, 1H), 4.81-4.59 (m, 1H), 3.78 (dd, J=7.6, 12.4 Hz, 1H), 2.74 (dd, J=4.4, 12.8 Hz, 1H), 1.83-1.73 (m, 1H), 1.21-1.15 (m, 1H).

### Intermediate 31

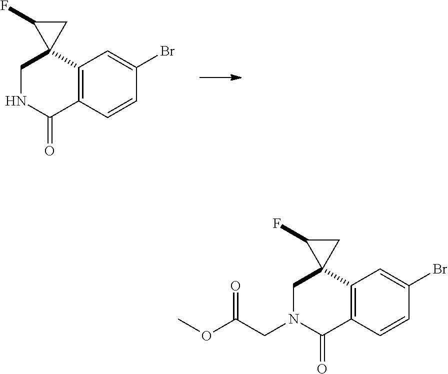

Methyl 2-[(2′s,4r)-6-bromo-2′-fluoro-1-oxospiro[3H-isoquinoline-4,1′-cyclopropane]-2-yl]acetate: To a solution of (2′s,4r)-6-bromo-2′-fluorospiro[2,3-dihydroisoquinoline-4,1′-cyclopropane]-1-one (200 mg, 0.74 mmol, Int. 29) in DMF (2.0 mL) at 0° C. was added NaH (45 mg, 1.11 mmol, 60% purity in mineral oil). The reaction mixture was stirred at 20° C. for 0.5 h followed by the addition of methyl 2-bromoacetate (227 mg, 1.48 mmol). The reaction mixture was stirred at 20° C. for 3 h. The reaction mixture was poured into sat. aq. NH₄Cl and ice water mixture (10 mL) and extracted with EtOAc (3×5 mL). The combined organic layers were washed with brine (5 mL), dried over anhydrous Na₂SO₄, filtered, and concentrated under reduced pressure. The residue was purified by silica gel column chromatography. LCMS: m/z=342.0, 344.0 [M+H]⁺. ¹H NMR (400 MHz, CDCl₃): δ 8.03 (d, J=4.4 Hz, 1H), 7.51 (d, J=8.4 Hz, 1H), 6.83 (s, 1H), 4.73-4.54 (m, 2H), 4.13-4.08 (m, 1H), 4.03-3.94 (m, 1H), 3.77 (s, 3H), 3.44 (m, 1H), 1.63-1.55 (m, 1H), 1.44-1.33 (m, 1H).

### Intermediates 32 and 33

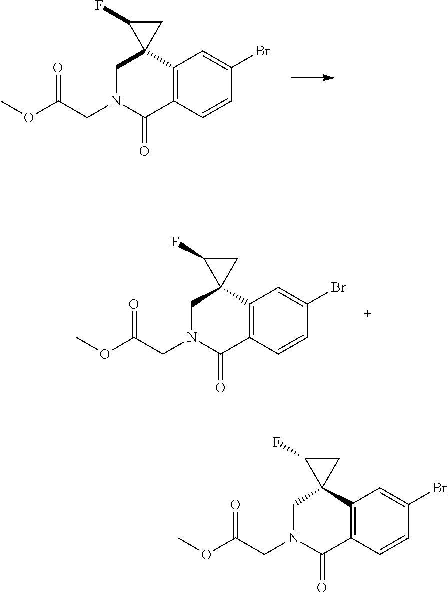

Methyl 2-[(2′s,4r)-6-bromo-2′-fluoro-1-oxospiro[3H-isoquinoline-4,1′-cyclopropane]-2-yl]acetate (Int. 31) could be further separated by chiral SFC (Column: Daicel Chiralpak AD (250 mm×30 mm, 10 μm particle size); Mobile Phase: A: CO₂, B: 0.1% NH₄OH in i-PrOH; Gradient: 38% B isocratic; Flow Rate: 64 g/min; Detection Wavelength: 220 nm; Column Temperature: 40° C.; System Back Pressure: 100 bar) to provide:

methyl 2-[(2′s,4r)-6-bromo-2′-fluoro-1-oxospiro[3H-isoquinoline-4,1′-cyclopropane]-2-yl]acetate (first eluting isomer, Int. 32): ¹H NMR (400 MHz, CDCl₃): δ 8.02 (d, J=8.0 Hz, 1H), 7.51 (dd, J=1.6, 8.4 Hz, 1H), 6.83 (d, J=1.6 Hz, 1H), 4.73-4.54 (m, 2H), 4.10 (dd, J=2.0, 12.4 Hz, 1H), 4.00 (d, J=17.2 Hz, 1H), 3.77 (s, 3H), 3.44 (d, J=12.4 Hz, 1H), 1.60-1.57 (m, 1H), 1.44-1.33 (m, 1H).

methyl 2-[(2′s,4r)-6-bromo-2′-fluoro-1-oxospiro[3H-isoquinoline-4,1′-cyclopropane]-2-yl]acetate (second eluting isomer, Int. 33): ¹H NMR (400 MHz, CDCl₃): δ 8.02 (d, J=8.0 Hz, 1H), 7.51 (dd, J=1.6, 8.4 Hz, 1H), 6.83 (d, J=1.6 Hz, 1H), 4.73-4.54 (m, 2H), 4.10 (dd, J=2.0, 12.4 Hz, 1H), 4.00 (d, J=17.2 Hz, 1H), 3.77 (s, 3H), 3.44 (d, J=12.4 Hz, 1H), 1.60-1.57 (m, 1H), 1.44-1.33 (m, 1H).

Absolute stereochemistry of intermediates 32 and 33 were assigned based on stereochemical identification of a Table 1A compound disclosed herein. Intermediate 32 has been identified as methyl 2-((1R,2S)-6′-bromo-2-fluoro-1′-oxo-1′H-spiro[cyclopropane-1,4′-isoquinolin]-2′(3′H)-yl)acetate. Intermediate 32 has been identified as methyl 2-((1S,2R)-6′-bromo-2-fluoro-1′-oxo-1′H-spiro[cyclopropane-1,4′-isoquinolin]-2′(3′H)-yl)acetate.

### Intermediate 34

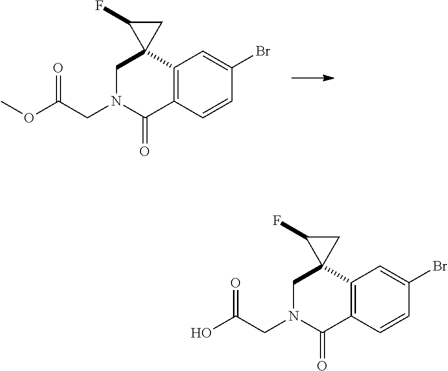

2-[(2′s,4r)-6-bromo-2′-fluoro-1-oxospiro[3H-isoquinoline-4,1′-cyclopropane]-2-yl]acetic acid: To a solution of methyl 2-[(2′s,4r)-6-bromo-2′-fluoro-1-oxospiro[3H-isoquinoline-4,1′-cyclopropane]-2-yl]acetate (150 mg, 0.44 mmol, Int. 33) in THF (2.0 mL) and water (2.0 mL) was added LiOH·H₂O (46 mg, 1.10 mmol). The reaction mixture was stirred at 20° C. for 1 h. The reaction mixture was diluted with water (5 mL), extracted with MTBE (3 mL), and the organics were discarded. The aqueous was adjusted pH=3 by the addition of aq. HCl (3 M) and extracted with EtOAc (3×2 mL). The combined organics were washed with brine (5 mL), dried over anhydrous Na₂SO₄, filtered, and concentrated under reduced pressure to provide a residue that was used directly. LCMS: m/z=328.0, 330.0 [M+H]⁺.

Intermediate 34 has been identified as 2-((1S,2R)-6′-bromo-2-fluoro-1′-oxo-1′H-spiro[cyclopropane-1,4′-isoquinolin]-2′(3′H)-yl)acetic acid.

### Intermediate 35

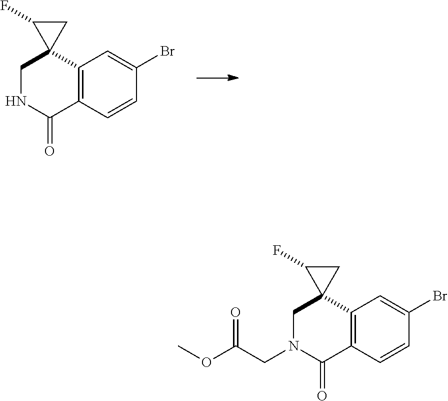

Methyl 2-[(2′r,4r)-6-bromo-2′-fluoro-1-oxospiro[3H-isoquinoline-4,1′-cyclopropane]-2-yl]acetate: To a solution of (2′r,4r)-6-bromo-2′-fluorospiro[2,3-dihydroisoquinoline-4,1′-cyclopropane]-1-one (210 mg, 0.77 mmol, Int. 30) in DMF (2.0 mL) at 0° C. was added NaH (93 mg, 2.33 mmol, 60% purity in mineral oil). The reaction mixture was stirred at 0° C. for 0.5 h followed by the addition of methyl 2-bromoacetate (119 mg, 0.78 mmol). The reaction mixture was stirred at 20° C. for 3 h. The reaction mixture was poured into a mixture of sat. aq. NH₄Cl and ice water (10 mL) and extracted with EtOAc (3×5 mL). The combined organic layers were washed with brine (5 mL), dried over anhydrous Na₂SO₄, filtered, and concentrated under reduced pressure. The residue was purified by silica gel column chromatography. ¹H NMR (400 MHz, CDCl₃): δ 8.03 (d, J=8.4 Hz, 1H), 7.54 (dd, J=1.2, 8.4 Hz, 1H), 7.26 (s, 1H), 4.90-4.70 (m, 1H), 4.44-4.25 (m, 2H), 3.97 (dd, J=7.6, 12.4 Hz, 1H), 3.79 (s, 3H), 2.72-2.65 (m, 1H), 1.87-1.71 (m, 1H), 1.23-1.10 (m, 1H).

### Intermediate 36

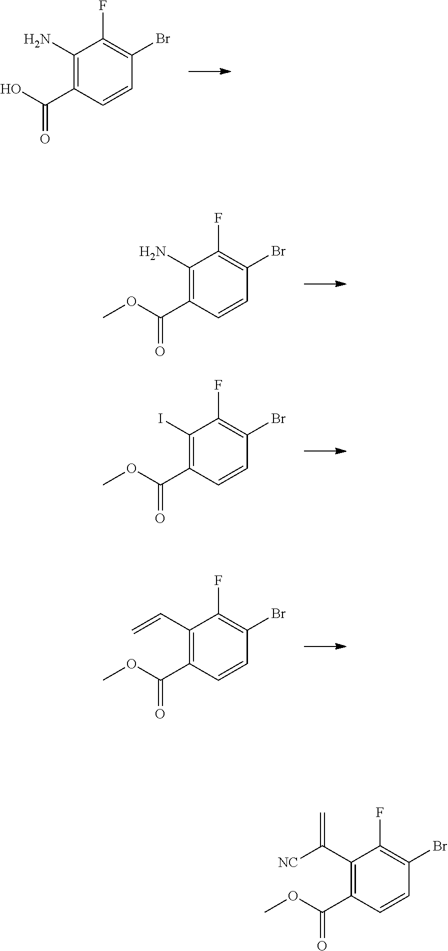 [SMILES: Nc1c(C(=O)O)ccc(Br)c1F ; COC(=O)c1ccc(Br)c(F)c1N ; COC(=O)c1ccc(Br)c(F)c1I ; C=Cc1c(C(=O)OC)ccc(Br)c1F ; C=C(C#N)c1c(C(=O)OC)ccc(Br)c1F]

methyl 2-amino-4-bromo-3-fluorobenzoate: To a solution of 2-amino-4-bromo-3-fluoro-benzoic acid (40.0 g, 170 mmol) in DCM (500 mL) and MeOH (500 mL) at 0° C. was added TMSCHN₂ (2 M in n-hexane, 427 mL, 854 mmol). The mixture was stirred for 16 h at 15° C., cooled to 0° C., and TMSCHN₂ (2 M in hexane, 171 mL) was added. The mixture was stirred for another 16 h at 15° C. The mixture was quenched by addition of sat. aq. NH₄Cl (500 mL) and H₂O (500 mL). The mixture was concentrated under reduced pressure to remove organic solvent. The white precipitate was filtered and dried under reduced pressure. The filter cake was then suspended in n-hexane (300 mL), stirred for 10 min, filtered, and dried under reduced pressure to provide a residue that was used directly. LCMS: m/z=247.9, 249.9 [M+H]⁺.

methyl 4-bromo-3-fluoro-2-iodobenzoate: To a solution of methyl 2-amino-4-bromo-3-fluoro-benzoate (42.0 g, 170 mmol) in H₂SO₄ (420 mL) and MeCN (420 mL) at 0° C. was added a solution of NaNO₂ (14 g, 203 mmol) in H₂O (60 mL). The mixture was stirred for 1 h at 0° C. followed by the addition of a solution of KI (56 g, 338 mmol) in H₂O (60 mL). The mixture was stirred for 15 h at 15° C. The mixture was cooled to 0° C., diluted with sat. aq. Na₂S₂O₃ (1000 mL), and extracted with MTBE (4×500 mL). The combined organic layers were washed with brine (500 mL), dried over anhydrous Na₂SO₄, filtered, concentrated under reduced pressure, and purified by silica gel column chromatography.

methyl 4-bromo-3-fluoro-2-vinylbenzoate: To a solution of methyl 4-bromo-3-fluoro-2-iodo-benzoate (12.0 g, 33.0 mmol) and potassium hydride:trifluoro(vinyl)boron (5.6 g, 41.7 mmol) in 1,4-dioxane (300 mL) were added CsF (20.3 g, 133.7 mmol) and Pd(dppf)Cl₂ (4.89 g, 6.69 mmol). The mixture was stirred for 16 h at 100° C. The mixture was filtered, the filtrate was diluted with water (1000 mL) and extracted with EtOAc (3×300 mL). The combined organic layers were washed with brine (100 mL), dried over anhydrous Na₂SO₄, filtered, concentrated under reduced pressure, and purified by silica gel column chromatography. ¹H NMR (400 MHz, CDCl₃): δ 7.56-7.47 (m, 2H), 7.01 (dd, 1=11.6, 18 Hz, 1H), 5.81-5.72 (m, 1H), 5.66 (td, J=1.2, 12.8 Hz, 1H), 3.90 (s, 3H).

methyl 4-bromo-2-(1-cyanovinyl)-3-fluoro-benzoate (Int. 36): To a solution of 6,6′-dimethyl-2,2′-bipyridine (711 mg, 3.86 mmol), Cu₂O (2.76 g, 19.3 mmol) and SelectFluor (10.3 g, 29.0 mmol) in acetone (60 mL) and water (30 mL) at 0° C. were added a solution of methyl 4-bromo-3-fluoro-2-vinyl-benzoate (5.0 g, 19.3 mmol) in acetone (5 mL) and TMSCN (3.83 g, 38.6 mmol, 4.83 mL). The mixture was stirred for 16 h at 20° C. The mixture was quenched with H₂O (100 mL) and extracted with EtOAc (5×30 mL). The combined organic layers were washed with brine (30 mL), dried over anhydrous Na₂SO₄, filtered, concentrated under reduced pressure, and purified by silica gel column chromatography. LCMS: m/z=283.9, 285.9 [M+H]⁺.

### Intermediate 37 and 38

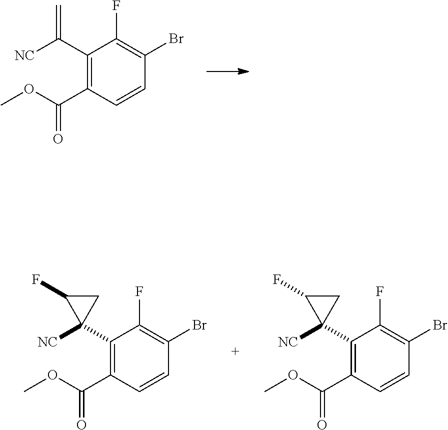

methyl 4-bromo-3-fluoro-2-[(1s,2r)-1-cyano-2-fluoro-cyclopropyl]benzoate and methyl 4-bromo-3-fluoro-2-[(1s,2s)-1-cyano-2-fluoro-cyclopropyl]benzoate: To a solution of methyl 4-bromo-2-(1-cyanovinyl)-3-fluoro-benzoate (6.0 g, 21.1 mmol, Int. 36) and (fluoromethyl)(phenyl)(2,3,4,5-tetramethylphenyl)sulfonium tetrafluoroborate (11.5 g, 31.7 mmol) in THF (80 mL) at 0° C. was added NaH (3.38 g, 84.5 mmol, 60% purity). The mixture was stirred for 1 h at 20° C. The mixture was cooled to 0° C., diluted with sat. aq. NH₄Cl (150 mL), and extracted with EtOAc (3×50 mL). The combined organic layers were washed with brine (50 mL), dried over anhydrous Na₂SO₄, filtered, concentrated under reduced pressure, and purified by silica gel column chromatography to provide:

methyl 4-bromo-3-fluoro-2-[(1s,2r)-1-cyano-2-fluoro-cyclopropyl]benzoate (Int. 37): ¹H NMR (400 MHz, CDCl₃): δ 7.73-7.66 (m, 2H), 4.83-4.63 (m, 1H), 4.03 (s, 3H), 2.39-2.28 (m, 1H), 1.67-1.57 (m, 1H).

methyl 4-bromo-3-fluoro-2-[(1s,2s)-1-cyano-2-fluoro-cyclopropyl]benzoate (Int. 38): 1H NMR (400 MHz, CDCl₃): δ 7.74-7.67 (m, 2H), 5.29-5.06 (m, 1H), 3.99 (s, 3H), 2.37-2.29 (m, 1H), 1.78-1.67 (m, 1H).

### Intermediate 39

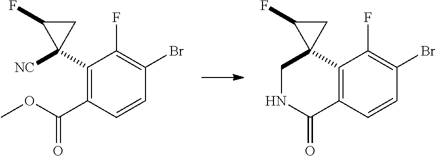

(2′s,4r)-6-bromo-2′,5-difluorospiro[2,3-dihydroisoquinoline-4,1′-cyclopropane]-1-one: To a solution of methyl 4-bromo-3-fluoro-2-[(1s,2r)-1-cyano-2-fluoro-cyclopropyl]benzoate (5.8 g, 18.3 mmol, Int. 37) and CoCl₂ (9.5 g, 73.4 mmol) in MeOH (180 mL) and H₂O (4.5 mL) at 0° C. was added NaBH₄ (2.78 g, 73.4 mmol). The mixture was stirred for 2 h at 20° C. Additional NaBH₄ (694 mg, 18.3 mmol) was then added to the solution. The mixture was stirred for a further 1 h. The mixture was quenched by sat. aq. NH₄Cl (200 mL) at 0° C., and extracted with EtOAc (3×60 mL). The combined organic layers were washed with brine (60 mL), dried over anhydrous Na₂SO₄, filtered, concentrated under reduced pressure and purified by silica gel column chromatography. LCMS: m/z=287.9, 289.9 [M+H]⁺.

### Intermediate 40 and 41

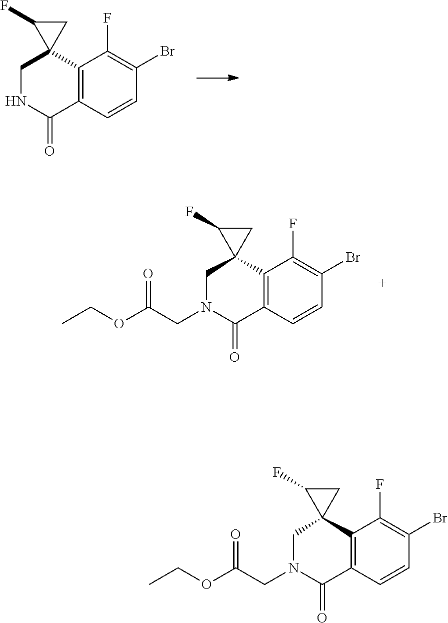

ethyl 2-[(2′s,4r)-6-bromo-2′,5-difluoro-1-oxo-spiro[3H-isoquinoline-4,1′-cyclopropane]-2-yl]acetate: To a solution of (2′s,4r)-6-bromo-2′,5-difluorospiro[2,3-dihydroisoquinoline-4,1′-cyclopropane]-1-one (1.2 g, 4.17 mmol, Int. 39) in DMF (20 mL) at 0° C. was added Cs₂CO₃ (2.71 g, 8.33 mmol). The mixture was stirred for 0.5 h. Ethyl 2-iodoacetate (1.34 g, 6.25 mmol, 0.74 mL) was added to the solution. The mixture was stirred for 2 h at 15° C. The mixture was quenched with H₂O (45 mL) and extracted with EtOAc (3×15 mL). The combined organic layers were washed with brine (10 mL), dried over anhydrous Na₂SO₄, filtered, and concentrated under reduced pressure. The mixture was purified by silica gel column chromatography. The mixture was further purified by reverse-phase preparative HPLC. The mixture was further purified by chiral SFC (Column: Daicel Chiralpak AS (250 mm×30 mm, 10 μm particle size); Mobile phase: A: CO₂ and B: i-PrOH; Gradient: 15% B isocratic; Flowrate: 55 g/min; Detection wavelength: 220 nm; Column temperature: 35° C.; System back pressure: 100 bar) to provide:

ethyl 2-[(2′s,4r)-6-bromo-2′,5-difluoro-1-oxo-spiro[3H-isoquinoline-4,1′-cyclopropane]-2-yl]acetate (first eluting isomer, Int. 40): LCMS: m/z=373.9, 375.9 [M+H]⁺. ¹H NMR (400 MHz, CDCl₃): δ 7.87 (d, J=8.0 Hz, 1H), 7.58-7.51 (m, 1H), 5.48-5.23 (m, 1H), 4.60 (d, J=17.2 Hz, 1H), 4.23 (q, J=7.2 Hz, 2H), 4.05 (d, J=17.2 Hz, 1H), 3.79 (dd, J=2.4, 12.8 Hz, 1H), 3.63 (d, J=12.8 Hz, 1H), 1.89 (td, J=7.2, 14.0 Hz, 1H), 1.55-1.40 (m, 1H), 1.30 (t, J=7.2 Hz, 3H).

ethyl 2-[(2′s,4r)-6-bromo-2′,5-difluoro-1-oxo-spiro[3H-isoquinoline-4,1′-cyclopropane]-2-yl]acetate (second eluting isomer, Int. 41): LCMS: m/z=373.9, 375.9 [M+H]⁺. ¹H NMR (400 MHz, CDCl₃): δ 7.87 (d, J=8.0 Hz, 1H), 7.58-7.51 (m, 1H), 5.48-5.23 (m, 1H), 4.60 (d, J=17.2 Hz, 1H), 4.23 (q, J=7.2 Hz, 2H), 4.05 (d, J=17.2 Hz, 1H), 3.79 (dd, J=2.4, 12.8 Hz, 1H), 3.63 (d, J=12.8 Hz, 1H), 1.89 (td, J=7.2, 14.0 Hz, 1H), 1.55-1.40 (m, 1H), 1.30 (t, J=7.2 Hz, 3H).

### Intermediate 42 and 43

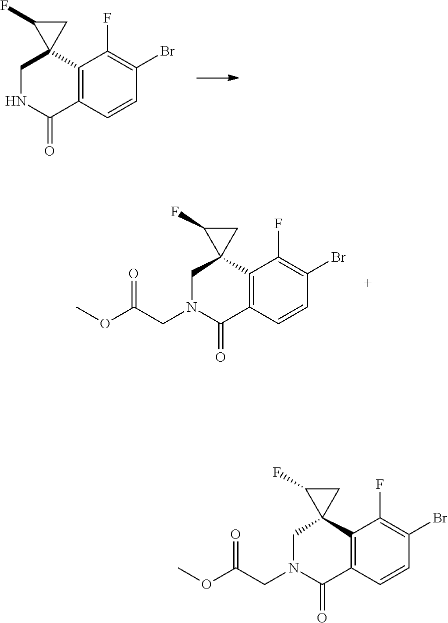

methyl 2-[(2′s,4r)-6-bromo-2′,5-difluoro-1-oxo-spiro[3H-isoquinoline-4,1′-cyclopropane]-2-yl]acetate: To a solution of (2′s,4r)-6-bromo-2′,5-difluorospiro[2,3-dihydroisoquinoline-4,1′-cyclopropane]-1-one (31.0 g, 107 mmol, Int. 39) and NaI (1.61 g, 10.8 mmol) in DMF (350 mL) at 0° C. were added Cs₂CO₃ (70 g, 215 mmol) and methyl 2-bromoacetate (19.7 g, 129 mmol). The mixture was stirred at 20° C. for 16 h. The reaction mixture was quenched by addition of aq. HCl (1 M, 700 mL) at 0° C., and extracted with EtOAc (3×200 mL). The combined organic layers were washed with brine (3×350 mL), dried over Na₂SO₄, filtered and concentrated under reduced pressure. The residue was purified by reverse-phase HPLC. The mixture was further by chiral SFC (Column: Daicel Chiralpak IG (250 mm×50 mm, 10 μm particle size); Mobile phase: A: CO₂ and B: MeOH; Gradient: 35% B isocratic; Flow rate: 200 g/min; Detection wavelength: 220 nm; Column temperature: 40° C.; System back pressure: 100 bar) to provide:

methyl 2-[(2′s,4r)-6-bromo-2′,5-difluoro-1-oxo-spiro[3H-isoquinoline-4,1′-cyclopropane]-2-yl]acetate (first eluting isomer, Int. 42): LCMS: m/z=359.9, 361.9 [M+H]⁺. ¹H NMR (400 MHz, CDCl₃): δ 7.87 (dd, J=1.0, 8.4 Hz, 1H), 7.54 (dd, J=6.4, 8.4 Hz, 1H), 5.44-5.23 (m, 1H), 4.60 (d, J=17.6 Hz, 1H), 4.12-4.00 (m, 1H), 3.81-3.77 (m, 4H), 3.62 (d, J=12.8 Hz, 1H), 1.93-1.87 (m, 1H), 1.54-1.41 (m, 1H).

methyl 2-[(2′s,4r)-6-bromo-2′,5-difluoro-1-oxo-spiro[3H-isoquinoline-4,1′-cyclopropane]-2-yl]acetate (second eluting isomer, Int. 43): LCMS: m/z=359.9, 361.9 [M+H]⁺. ¹H NMR (400 MHz, CDCl₃): δ 7.87 (dd, J=0.8, 8.4 Hz, 1H), 7.54 (dd, J=6.4, 8.4 Hz, 1H), 5.46-5.23 (m, 1H), 4.60 (d, J=17.6 Hz, 1H), 4.08 (d, J=17.6 Hz, 1H), 3.81 (d, J=2.6 Hz, 1H), 3.78 (s, 3H), 3.63 (d, J=12.9 Hz, 1H), 1.93-1.87 (m, 1H), 1.55-1.40 (m, 1H).

### Intermediate 44

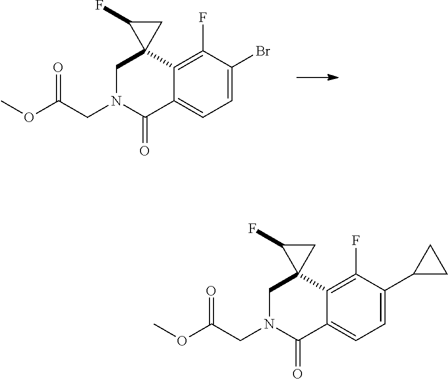

methyl 2-[(2′s,4r)-6-cyclopropyl-2′,5-difluoro-1-oxo-spiro[3H-isoquinoline-4,1′-cyclopropane]-2-yl]acetate: To a solution of methyl 2-[(2′s,4r)-6-bromo-2′,5-difluoro-1-oxo-spiro[3H-isoquinoline-4,1′-cyclopropane]-2-yl]acetate (50 mg, 0.14 mmol, Int. 43) and potassium cyclopropyltrifluoroborate (62 mg, 0.42 mmol) in 1,4-dioxane (1.0 mL) and H₂O (0.1 mL) were added CsF (63 mg, 0.42 mmol) and Pd(dppf)Cl₂ (10 mg, 0.014 mmol). The mixture was stirred at 100° C. for 16 h. The reaction mixture was quenched with H₂O (6 mL) and extracted with EtOAc (3×2 mL). The combined organic layers were washed with brine (3 mL), dried over anhydrous Na₂SO₄, filtered, concentrated under reduced pressure, and purified by silica gel column chromatography. LCMS: m/z=322.0 [M+H]⁺.

### Intermediate 45

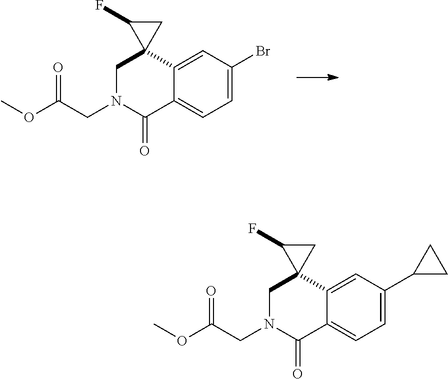

methyl 2-[(2′s,4r)-6-cyclopropyl-2′-fluoro-1-oxo-spiro[3H-isoquinoline-4,1′-cyclopropane]-2-yl]acetate: To a solution of methyl 2-[(2′s,4r)-6-bromo-2′-fluoro-1-oxospiro[3H-isoquinoline-4,1′-cyclopropane]-2-yl]acetate (100 mg, 0.30 mmol, Int. 33) and cyclopropylboronic acid (75 mg, 0.88 mmol) in 1,4-dioxane (2 mL) were added Pd(dppf)Cl₂ (21 mg, 0.03 mmol) and CsF (133 mg, 0.88 mmol). The mixture was stirred at 100° C. for 6 h. The mixture was added H₂O (5 mL) and extracted with EtOAc (3×5 mL). The combined organic layers were washed with brine (5 mL), dried over anhydrous Na₂SO₄, filtered, concentrated under reduced pressure, and purified by silica gel column chromatography. ¹H NMR (400 MHz, CDCl₃): δ 8.04 (d, J=8.4 Hz, 1H), 7.01-6.98 (m, 1H), 6.39 (s, 1H), 4.72-4.52 (m, 2H), 4.07 (dd, J=2.4, 12.0 Hz, 1H), 4.01 (d, J=18.0 Hz, 1H), 3.76 (s, 3H), 3.43 (d, J=12.4 Hz, 1H), 1.97-1.83 (m, 1H), 1.26 (s, 2H), 1.08-1.00 (m, 2H), 0.76-0.70 (m, 2H).

Intermediate 45 has been identified as methyl 2-((1S,2R)-6′-cyclopropyl-2-fluoro-1′-oxo-1′H-spiro[cyclopropane-1,4′-isoquinolin]-2′(3′H)-yl)acetate.

### Intermediate 46 and 47

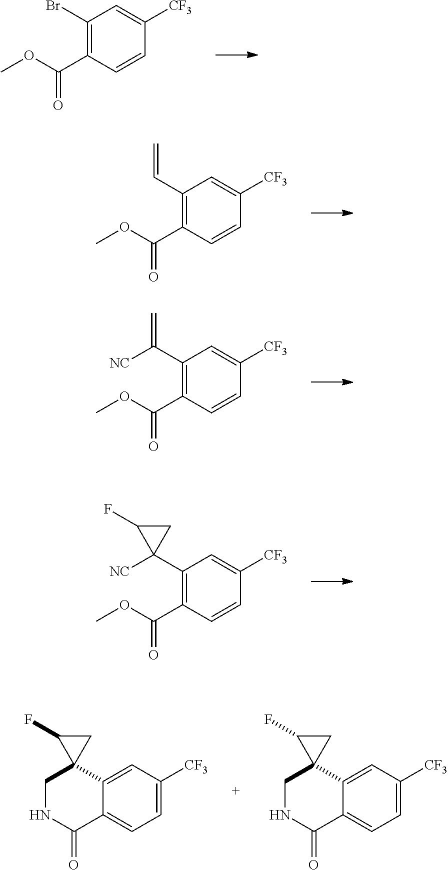

methyl 4-(trifluoromethyl)-2-vinylbenzoate: To a solution of methyl 2-bromo-4-(trifluoromethyl)benzoate (100 g, 353 mmol) in 1,4-dioxane (2000 mL) was added potassium vinyltrifluoroborate (52 g, 388 mmol), CsF (161 g, 1.06 mol) and Pd(dppf)Cl₂ (12.9 g, 17.7 mmol). The mixture was stirred at 90° C. for 6 h. The mixture was diluted with H₂O (2000 mL) and extracted with EtOAc (3×700 mL). The combined organic layers were washed with brine (1500 mL), dried over anhydrous Na₂SO₄, filtered, and concentrated under reduced pressure. The residue was purified by silica gel column chromatography.

methyl 2-(1-cyanovinyl)-4-(trifluoromethyl)benzoate: To a solution of Cu₂O (31.0 g, 217 mmol), 6,6′-dimethyl-2,2′-bipyridine (8.0 g, 43 mmol) and SelectFluor (115 g, 325 mmol) in acetone (1 L) and H₂O (500 mL) were added dropwise methyl 4-(trifluoromethyl)-2-vinylbenzoate (50 g, 217 mmol) and TMSCN (64.6 g, 652 mmol). The mixture was stirred at 20° C. for 20 h. The mixture was poured into aq. sat. NaHCO₃ (200 mL) and H₂O (800 mL). The mixture was filtered and the filtrate was extracted with EtOAc (3×300 mL). The combined organic layers were washed with brine (600 mL), dried over anhydrous Na₂SO₄, filtered, and concentrated under reduced pressure. The residue was purified by silica gel column chromatography. LCMS: m/z=256.0 [M+H]⁺.

methyl 2-[(1r,2s)-1-cyano-2-fluoro-cyclopropyl]-4-(trifluoromethyl)benzoate and methyl 2-[(1s,2s)-1-cyano-2-fluoro-cyclopropyl]-4-(trifluoromethyl)benzoate: To a solution of methyl 2-(1-cyanovinyl)-4-(trifluoromethyl)benzoate (11.0 g, 43.1 mmol) in THF (200 mL) at 0° C. were added (fluoromethyl)(phenyl)(2,3,4,5-tetramethylphenyl)sulfonium tetrafluoroborate (31.0 g, 86 mmol) and NaH (6.9 g, 172 mmol, 60% purity). The mixture was stirred at 20° C. for 1 h. The reaction mixture was poured into H₂O (30 mL) and extracted with EtOAc (3×10 mL). The combined organic layers were washed with brine (30 mL), dried over anhydrous Na₂SO₄, filtered, and concentrated under reduced pressure. The residue was purified by silica gel column chromatography. LCMS: m/z=288.0 [M+H]⁺.

(2′s,4r)-2′-fluoro-6-(trifluoromethyl)spiro[2,3-dihydroisoquinoline-4,1′-cyclopropane]-1-one and (2′s,4s)-2′-fluoro-6-(trifluoromethyl)spiro[2,3-dihydroisoquinoline-4,1′-cyclopropane]-1-one: To a solution of methyl 2-[(1r,2s)-1-cyano-2-fluoro-cyclopropyl]-4-(trifluoromethyl)benzoate and methyl 2-[(1s,2s)-1-cyano-2-fluoro-cyclopropyl]-4-(trifluoromethyl)benzoate (12.0 g, 41.0 mmol) in MeOH (120 mL) was added Raney-Ni (5.0 g, 58.0 mmol). The mixture was stirred at 20° C. for 1 h under H₂ (30 psi). The mixture was filtered, and the filtrate was concentrated under reduced pressure. The residue was purified by silica gel column chromatography to provide:

(2′s,4r)-2′-fluoro-6-(trifluoromethyl)spiro[2,3-dihydroisoquinoline-4,1′-cyclopropane]-1-one (Int. 46): LCMS: m/z=260.0 [M+H]⁺. ¹H NMR (400 MHz, CDCl₃): δ 8.27 (d, J=8.0 Hz, 1H), 7.65 (d, J=8.0 Hz, 1H), 7.04 (br s, 1H), 6.95 (s, 1H), 4.75-4.52 (m, 1H), 3.91 (d, J=12.8 Hz, 1H), 3.58 (dd, J=4.4, 13.2 Hz, 1H), 1.61-1.72 (m, 1H), 1.47-1.36 (m, 1H);

(2′s,4s)-2′-fluoro-6-(trifluoromethyl)spiro[2,3-dihydroisoquinoline-4,1′-cyclopropane]-1-one (Int 47): LCMS: m/z=260.0 [M+H]⁺. ¹H NMR (400 MHz, CDCl₃): δ 8.27 (d, J=8.4 Hz, 1H), 7.67 (dd, J=0.8, 8.0 Hz, 1H), 7.48 (br s, 1H), 7.37 (s, 1H), 4.89-4.62 (m, 1H), 3.81 (dd, J=7.6, 12.8 Hz, 1H), 2.81 (dd, J=4.8, 13.2 Hz, 1H), 1.93-1.79 (m, 1H), 1.25-1.18 (m, 1H).

### Intermediate 48

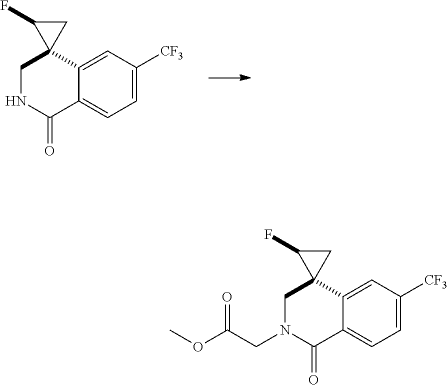

methyl 2-[(2′s,4r)-2′-fluoro-1-oxo-6-(trifluoromethyl)spiro[3H-isoquinoline-4,1′-cyclopropane]-2-yl]acetate: To a solution of (2′s,4r)-2′-fluoro-6-(trifluoromethyl)spiro[2,3-dihydroisoquinoline-4,1′-cyclopropane]-1-one (2.4 g, 9.3 mmol, Int. 46) in DMF (30 mL) at 0° C. was added NaH (560 mg, 13.9 mmol, 60% purity). The mixture was stirred for 0.5 h at 0° C. To the mixture was added methyl 2-bromoacetate (2.83 g, 18.5 mmol) at 0° C. The mixture was stirred at 20° C. for 2 h. The mixture was poured into sat. aq. NH₄Cl (30 mL) and extracted with EtOAc (3×10 mL). The combined organic layers were washed with brine (20 mL), dried over anhydrous Na₂SO₄, filtered, and concentrated under reduced pressure. The residue was purified by silica gel column chromatography. LCMS: m/z=332.2 [M+H]⁺.

### Intermediate 49

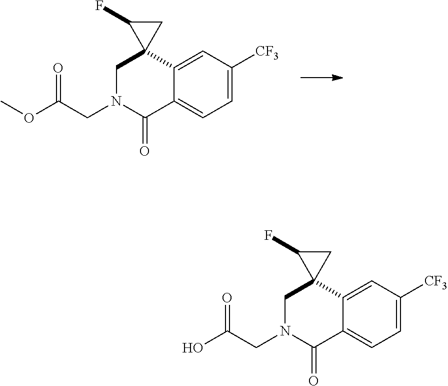

2-[(2′s,4r)-2′-fluoro-1-oxo-6-(trifluoromethyl)spiro[3H-isoquinoline-4,1′-cyclopropane]-2-yl]acetic acid: To a solution of methyl 2-[(2′s,4r)-2′-fluoro-1-oxo-6-(trifluoromethyl)spiro[3H-isoquinoline-4,1′-cyclopropane]-2-yl]acetate (150 mg, 0.45 mmol, Int. 48) in THF (2.0 mL) and H₂O (2.0 mL) was added LiOH·H₂O (47 mg, 1.1 mmol). The mixture was stirred at 20° C. for 2 h. The mixture was diluted with H₂O (3 mL) and extracted with MTBE (2 mL). The aqueous phase was adjusted to pH=3 by addition of aq. HCl (3 M). The mixture was extracted with EtOAc (3×2 mL). The combined organic layers were washed with brine (3 mL), dried over anhydrous Na₂SO₄, filtered, and concentrated under reduced pressure to give a crude residue that was used directly. LCMS: m/z=317.9 [M+H]⁺.

### Intermediate 50

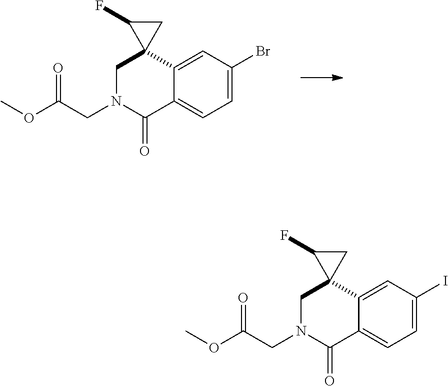

methyl 2-[(2′s,4r)-2′-fluoro-6-iodo-1-oxo-spiro[3H-isoquinoline-4,1′-cyclopropane]-2-yl]acetate: A mixture of methyl 2-[(2′s,4r)-6-bromo-2′-fluoro-1-oxospiro[3H-isoquinoline-4,1′-cyclopropane]-2-yl]acetate (200 mg, 0.58 mmol, Int. 33), CuI (22 mg, 0.12 mmol), NaI (350 mg, 2.34 mmol) and (1R,2R)—N1,N2-dimethylcyclohexane-1,2-diamine (33 mg, 0.23 mmol) in toluene (4 mL) was degassed and purged with N₂. The mixture was stirred at 130° C. for 32 h. The reaction mixture was washed with H₂O (5 mL) and extracted with EtOAc (3×5 mL). The combined organic layers were washed with brine (5 mL), dried over anhydrous Na₂SO₄, filtered, and concentrated under reduced pressure to provide a residue that was used directly. ¹H NMR (400 MHz, CDCl₃): δ 7.86 (d, J=8.4 Hz, 1H), 7.73 (dd, J=8.4, 1.6 Hz, 1H), 7.04 (d, J=1.2 Hz, 1H), 4.66-4.53 (m, 2H), 4.09 (dd, J=12.4, 1.6 Hz, 1H), 3.99 (d, J=17.2 Hz, 1H), 3.77 (s, 3H), 3.43 (d, J=12.8 Hz, 1H), 1.60-1.56 (m, 1H), 1.33-1.43 (m, 1H).

Intermediate 50 has been identified as methyl 2-((1S,2R)-2-fluoro-6′-iodo-1′-oxo-1′H-spiro[cyclopropane-1,4′-isoquinolin]-2′(3′H)-yl)acetate.

### Intermediate 51

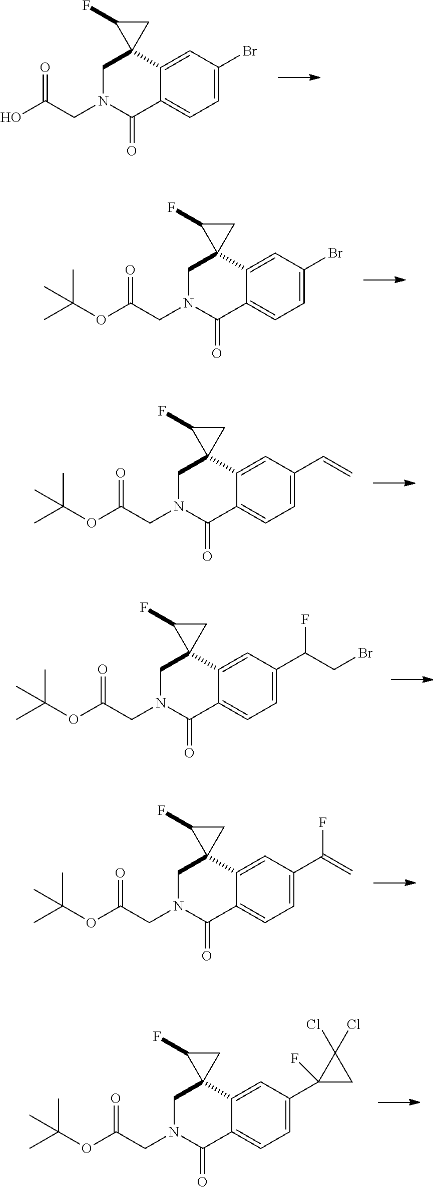

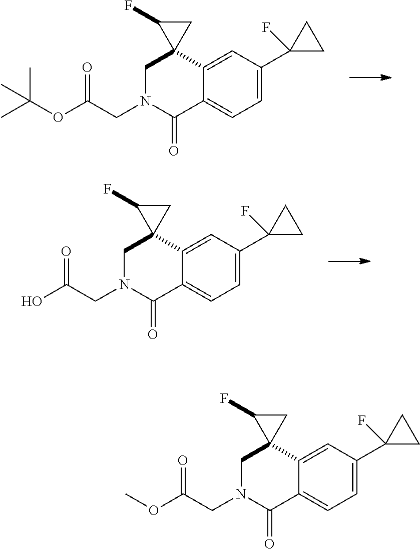

tert-butyl 2-[(2′s,4r)-6-bromo-2′-fluoro-1-oxospiro[3H-isoquinoline-4,1′-cyclopropane]-2-yl]acetate: To a solution of 2-[(2′s,4r)-6-bromo-2′-fluoro-1-oxospiro[3H-isoquinoline-4,1′-cyclopropane]-2-yl]acetic acid (1.25 g, 3.81 mmol, Int. 34) in DCM (20 mL) at 0° C. were added DMAP (465 mg, 3.81 mmol), DCC (865 mg, 4.19 mmol), and t-BuOH (424 mg, 5.71 mmol). The mixture was stirred at 20° C. for 16 h. The mixture was filtered and the filtrate was washed with aq. sat. Na₂CO₃ (3×200 mL). The combined organic layers were washed with brine (2×20 mL), dried over anhydrous Na₂SO₄, filtered, concentrated under reduced pressure, and purified by silica gel column chromatography. ¹H NMR (400 MHz, CDCl₃): δ 8.03 (d, J=8.0 Hz, 1H), 7.50 (dd, J=2.0, 8.4 Hz, 1H), 6.82 (d, J=2.0 Hz. 1H), 4.73-4.51 (m, 2H), 4.07 (dd, J=1.6, 12.4 Hz, 1H), 3.85 (d, J=17.2 Hz, 1H), 3.43 (d, J=12.4 Hz, 1H), 1.58-1.54 (m, 1H), 1.48 (s, 9H), 1.44-1.33 (m, 1H).

tert-butyl 2-[(2′s,4r)-2′-fluoro-1-oxo-6-vinylspiro[3H-isoquinoline-4,1′-cyclopropane]-2-yl]acetate: To a solution of tert-butyl 2-[(2′s,4r)-6-bromo-2′-fluoro-1-oxospiro[3H-isoquinoline-4,1′-cyclopropane]-2-yl]acetate (500 mg, 1.30 mmol), potassium trifluoro(vinyl)borate (436 mg, 3.25 mmol) in 1,4-dioxane (10 mL) were added CsF (593 mg, 3.90 mmol) and Pd(dppf)Cl₂ (95 mg, 0.13 mmol). The mixture was stirred at 90° C. for 3 h. The reaction mixture was poured into H₂O (20 mL) and extracted with EtOAc (4×15 mL). The combined organic layers were washed with brine (2×10 mL), dried over anhydrous Na₂SO₄, filtered, concentrated under reduced pressure, and purified by silica gel column chromatography. LCMS: m/z=276.2 [M-tBu+H]⁺. ¹H NMR (400 MHz, CDCl₃): δ 8.13 (d, J=8.0 Hz, 1H), 7.41 (dd, J=1.6, 8.0 Hz, 1H), 6.75-6.68 (m, 1H), 6.66 (d, J=1.6 Hz, 1H), 5.82 (d, J=17.6 Hz, 1H), 5.37 (d, J=11.2 Hz, 1H), 4.75-4.53 (m, 2H), 4.07 (dd, J=2.0, 12.8 Hz, 1H), 3.87 (d, J=17.2 Hz, 1H), 3.44 (d, J=12.8 Hz, 1H), 1.64-1.60 (m, 1H), 1.48 (s, 9H), 1.42-1.31 (m, 1H).

tert-butyl 2-[(2′s,4r)-2′-fluoro-1-oxo-6-(2-bromo-1-fluoroethyl)spiro[3H-isoquinoline-4,1′-cyclopropane]-2-yl]acetate: To a solution of tert-butyl 2-[(2′s,4r)-2′-fluoro-1-oxo-6-vinylspiro[3H-isoquinoline-4,1′-cyclopropane]-2-yl]acetate (1.04 g, 3.14 mmol) in DCM (20 mL) at 0° C. were added NBS (614 mg, 3.45 mmol) and triethylamine tris(hydrogen fluoride) (759 mg, 4.71 mmol). The mixture was stirred at 0° C. for 15 min then stirred at 20° C. for 16 h. The mixture was poured into H₂O (10 mL) and extracted with DCM (3×10 mL). The combined organic layers were washed with brine (30 mL), dried over anhydrous Na₂SO₄, filtered, concentrated under reduced pressure, and purified by silica gel column chromatography. LCMS: m/z=374.0, 376.0 [M-tBu+H]⁺. ¹H NMR (400 MHz, CDCl₃): δ 8.20 (d, J=8.0 Hz, 1H), 7.31 (br d, J=8.0 Hz, 1H), 6.70 (s, 1H), 5.73-5.55 (m, 1H), 4.75-4.52 (m, 2H), 4.15-4.04 (m, 1H), 3.87 (dd, J=4.8, 17.2 Hz, 1H), 3.68-3.57 (m, 2H), 3.46 (dd, J=6.4, 12.8 Hz, 1H), 1.66-1.59 (m. 1H), 1.49 (s, 9H), 1.45-1.35 (m, 1H).

tert-butyl 2-[(2′s,4r)-2′-fluoro-1-oxo-6-(1-fluorovinyl)spiro[3H-isoquinoline-4,1′-cyclopropane]-2-yl]acetate: To a solution of tert-butyl 2-[(2′s,4r)-2′-fluoro-1-oxo-6-(2-bromo-1-fluoroethyl)spiro[3H-isoquinoline-4,1′-cyclopropane]-2-yl]acetate (1.07 g, 2.49 mmol) in DMSO (20 mL) was added DBU (568 mg, 3.73 mmol). The mixture was stirred at 60° C. for 1 h. The reaction mixture was poured into H₂O (100 mL) and extracted with EtOAc (3×20 mL). The combined organic layers were washed with brine (30 mL), dried over anhydrous Na₂SO₄, filtered, concentrated under reduced pressure, and purified by silica gel column chromatography. LCMS: m/z=294.1 [M-t-Bu+H]⁺. ¹H NMR (400 MHz, CDCl₃): δ 8.27 (d, J=8.0 Hz, 1H), 7.64 (d, J=8.0 Hz, 1H), 6.93 (s, 1H), 5.23 (dd, J=3.6, 49.2 Hz, 1H), 5.07 (dd, J=3.6, 17.6 Hz, 1H), 4.87-4.62 (m, 2H), 4.26-4.11 (m, 1H), 4.02-3.91 (m, 1H), 3.64-3.50 (m, 1H), 1.74-1.67 (m, 1H), 1.58 (s, 9H), 1.54-1.44 (m, 1H).

tert-butyl 2-[(2′s,4r)-2′-fluoro-1-oxo-6-(2,2-dichloro-1-fluorocyclopropyl)spiro[3H-isoquinoline-4,1′-cyclopropane]-2-yl]acetate: To a solution of tert-butyl 2-[(2′s,4r)-2′-fluoro-1-oxo-6-(1-fluorovinyl)spiro[3H-isoquinoline-4,1′-cyclopropane]-2-yl]acetate (650 mg, 1.86 mmol) in CHCl₃ (15 mL) at 0° C. were added benzyltriethylammonium chloride (17 mg, 0.07 mmol) followed by aq. NaOH (4.47 g, 55.8 mmol, 50 wt %) dropwise. The mixture was stirred at 0° C. for 0.5 h. The mixture was then stirred at 20° C. for 3 h. The reaction mixture was poured into ice-cold H₂O (20 mL) and extracted with DCM (2×10 mL). The combined organic layers were washed with aq. HCl (20 mL, 0.1 M), aq. sat. NaHCO₃ (20 ml), and brine (20 mL), dried over anhydrous Na₂SO₄, filtered, and concentrated under reduced pressure. The residue was purified by silica gel column chromatography. LCMS: m/z=375.9, 377.0, 377.9 [M-t-Bu+H]⁺. ¹H NMR (400 MHz, CDCl₃): δ 8.22 (d, J=8.0 Hz, 1H), 7.40-7.31 (m, 1H), 6.89-6.81 (m, 1H), 4.76-4.56 (m, 2H), 4.18-4.05 (m, 1H), 3.87 (dd, J=3.2, 17.2 Hz, 1H), 3.52-3.42 (m, 1H), 2.36-2.19 (m, 2H), 1.70-1.62 (m, 1H), 1.49 (s, 9H), 1.46-1.36 (m, 1H).

tert-butyl 2-[(2′s,4r)-2′-fluoro-1-oxo-6-(1-fluorocyclopropyl)spiro[3H-isoquinoline-4,1′-cyclopropane]-2-yl]acetate: To a solution tert-butyl 2-[(2′s,4r)-2′-fluoro-1-oxo-6-(2,2-dichloro-1-fluorocyclopropyl)spiro[3H-isoquinoline-4,1′-cyclopropane]-2-yl]acetate (100 mg, 0.23 mmol) in tri-n-butyl-tin hydride (660 mg, 2.27 mmol) was added AIBN (3.80 mg, 0.023 mmol). The mixture was stirred at 160° C. for 3 h. The reaction mixture was poured into aq. sat. KF (20 mL). The mixture was stirred 1 h at 20° C., and extracted with EtOAc (4×10 mL). The combined organic layers were washed with brine (10 mL), dried over anhydrous Na₂SO₄, filtered, and concentrated under reduced pressure. The residue was purified by preparative silica gel thin-layer chromatography. LCMS: m/z=308.1 [M-tBu+H]⁺. ¹H NMR (400 MHz. CDCl₃): δ 8.13 (dd, J=0.8, 8.0 Hz, 1H), 6.99 (d, J=8.0 Hz, 1H), 6.71 (d, J=1.6 Hz, 1H), 4.77-4.53 (m, 2H), 4.08 (dd, J=2.0, 12.8 Hz, 1H), 3.87 (d, J=17.6 Hz, 1H), 3.45 (d, J=12.6 Hz, 1H), 1.59-1.51 (m, 2H), 1.48 (s, 9H), 1.43-1.32 (m, 2H), 1.14-1.09 (m, 2H).

2-[(2′s,4r)-2′-fluoro-1-oxo-6-(1-fluorocyclopropyl)spiro[3H-isoquinoline-4,1′-cyclopropane]-2-yl]acetic acid: To a solution of tert-butyl 2-[(2′s,4r)-2′-fluoro-1-oxo-6-(1-fluorocyclopropyl)spiro[3H-isoquinoline-4,1′-cyclopropane]-2-yl]acetate (190 mg, 0.52 mmol) in DCM (4.0 mL) was added formic acid (1.0 mL). The mixture was stirred at 40° C. for 12 h. The mixture was concentrated under reduced pressure to provide a residue that was used directly. LCMS: m/z=306.2 [M−H]⁻.

methyl 2-[(2′s,4r)-2′-fluoro-1-oxo-6-(1-fluorocyclopropyl)spiro[3H-isoquinoline-4,1′-cyclopropane]-2-yl]acetate (Int. 51): To a solution of 2-[(2′s,4r)-2′-fluoro-1-oxo-6-(1-fluorocyclopropyl)spiro[3H-isoquinoline-4,1′-cyclopropane]-2-yl]acetic acid (77 mg, 0.25 mmol) in DMF (2.0 mL) were added K₂CO₃ (52 mg, 0.38 mmol) and Mel (43 mg, 0.3 mmol). The mixture was stirred at 20° C. for 2 h. The reaction mixture was poured into H₂O (10 mL) and extracted with EtOAc (3×5 mL). The combined organic layers were washed with brine (2×10 mL), dried over anhydrous Na₂SO₄, filtered and concentrated under reduced pressure. The residue was purified by preparative silica gel thin-layer chromatography. LCMS: m/z=322.1 [M+H]⁺.

Intermediate 51 has been identified as methyl 2-((1S,2R)-2-fluoro-6′-(1-fluorocyclopropyl)-1′-oxo-1′H-spiro[cyclopropane-1,4′-isoquinolin]-2′(3′H)-yl)acetate.

### Intermediate 52

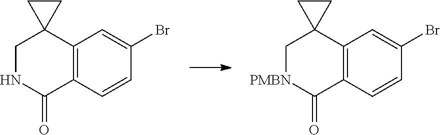

6-bromo-2-[(4-methoxyphenyl)methyl]spiro[3H-isoquinoline-4,1′-cyclopropane]-1-one: To a solution of 6′-bromo-2′,3′-dihydro-1′H-spiro[cyclopropane-1,4′-isoquinolin]-1′-one (5.0 g, 19.9 mmol, Int. 1) in THF (100 mL) at 0° C. were added PMBCl (3.73 g, 23.8 mmol) and NaH (1.59 g, 39.7 mmol, 60% purity). The mixture was stirred at 50° C. for 12 h. The reaction mixture was poured into aq. sat. NH₄Cl (100 mL) and extracted with EtOAc (3×50 mL). The combined organic layers were washed with brine (50 mL), dried over anhydrous Na₂SO₄, filtered and concentrated under reduced pressure. The residue was purified by silica gel column chromatography. ¹H NMR (400 MHz, CDCl₃): δ 8.05 (d, J=8.0 Hz, 1H), 7.45 (dd, J=2.0, 8.4 Hz, 1H), 7.24 (d, J=8.4 Hz, 2H), 6.95 (d, J=1.6 Hz, 1H), 6.89-6.85 (m, 2H), 4.71 (s, 2H), 3.81 (s, 3H), 3.20 (s. 2H), 1.04-0.99 (m, 2H), 0.77-0.72 (m, 2H).

### Intermediates 53 and 54

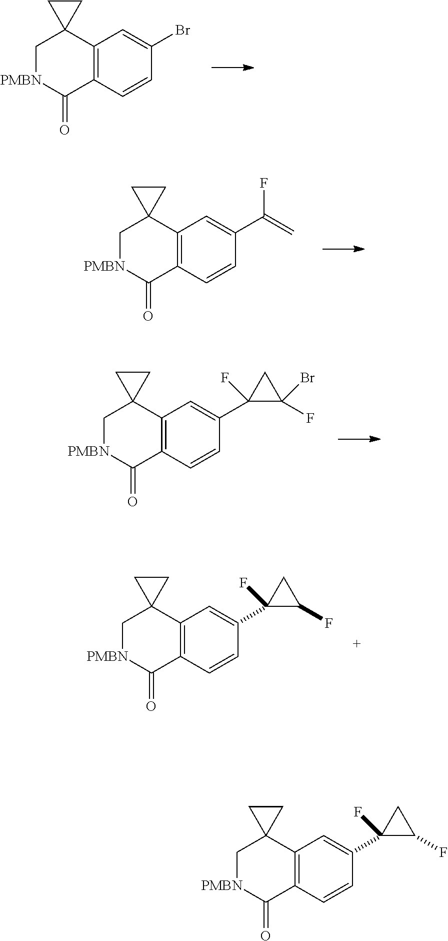

6′-(1-fluorovinyl)-2′-(4-methoxybenzyl)-2′,3′-dihydro-1′H-spiro[cyclopropane-1,4′-isoquinolin]-1′-one: To a solution of 6′-bromo-2′-(4-methoxybenzyl)-2′,3′-dihydro-1′H-spiro[cyclopropane-1,4′-isoquinolin]-1′-one (3 g, 8.06 mmol, Int. 52) and (1-fluorovinyl)(methyl)diphenylsilane (2.93 g, 12.1 mmol) in 1,3-dimethylimidazolidin-2-one (30 mL) were added CuI (306 mg, 1.61 mmol), Pd(dppf)Cl₂ (590 mg, 0.81 mmol), and CsF (3.06 g, 20.1 mmol). The mixture was stirred for 16 h. The reaction mixture was quenched by addition of H₂O (50 mL) and extracted with EtOAc (3×20 mL). The combined organic layers were washed with brine (3×10 mL), dried over anhydrous Na₂SO₄, filtered, and concentrated under reduced pressure. The residue was purified by silica gel column chromatography. LCMS: m/z=338.0 [M+H]⁺.

6′-(2-bromo-1,2-difluorocyclopropyl)-2′-(4-methoxybenzyl)-2′,3′-dihydro-1′H-spiro[cyclopropane-1,4′-isoquinolin]-1′-one: To a solution of 6′-(1-fluorovinyl)-2′-(4-methoxybenzyl)-2′,3′-dihydro-1′H-spiro[cyclopropane-1,4′-isoquinolin]-1′-one (800 mg, 2.37 mmol) in H₂O (2 mL) and DCM (10 mL) at 0° C. were added benzyltriethylammonium chloride (54 mg, 0.24 mmol), dibromo(fluoro)methane (2.27 g, 11.9 mmol) and aq. NaOH (190 mg, 2.37 mmol, 50 wt %). The mixture was stirred at 20° C. for 16 h. The reaction mixture was quenched by addition of aq. sat. NH₄Cl(30 mL) and extracted with EtOAc (3×20 mL). The combined organic layers were washed with brine (3×10 mL), dried over anhydrous Na₂SO₄, filtered, and concentrated under reduced pressure. The residue was purified by silica gel column chromatography. LCMS: m/z=447.9, 450.0 [M+H]⁺.

6-[(1r,2r)-1,2-difluorocyclopropyl]-2-[(4-methoxyphenyl)methyl]spiro[3H-isoquinoline-4,1′-cyclopropane]-1-one and 6-[(1r,2s)-1,2-difluorocyclopropyl]-2-[(4-methoxyphenyl)methyl]spiro[3H-isoquinoline-4,1′-cyclopropane]-1-one: A solution of 6′-(2-bromo-1,2-difluorocyclopropyl)-2′-(4-methoxybenzyl)-2′,3′-dihydro-1′H-spiro[cyclopropane-1,4′-isoquinolin]-1′-one (1.8 g, 4.02 mmol) and AIBN (65.93 mg, 0.401 mol) in toluene (15 mL) was degassed with N₂. To this mixture was added a solution of tributylstannane (4.32 g, 14.86 mmol) in toluene (2 mL). The reaction mixture was stirred at 70° C. for 3 h. The mixture was quenched by addition of aq. sat. KF (20 mL) and extracted with EtOAc (3×30 mL). The combined organic layers were washed with brine (3×20 mL), dried over anhydrous Na₂SO₄, filtered, and concentrated under reduced pressure. The residue was purified by silica gel column chromatography and further purified by reverse-phase preparative HPLC to provide:

6-[(1r,2s)-1,2-difluorocyclopropyl]-2-[(4-methoxyphenyl)methyl]spiro[3H-isoquinoline-4,1′-cyclopropane]-1-one (Int. 53): ¹H NMR (400 MHz, CDCl₃): δ=8.22 (d, J=8.0 Hz, 1H), 7.33 (br d, J=8.0 Hz, 1H), 7.28-7.25 (m, 2H), 6.91 (s, 1H), 6.87 (d, J=8.6 Hz, 2H), 5.19-4.90 (m, 1H), 4.73 (s, 2H), 3.81 (s, 3H), 3.23 (s, 2H), 1.90-1.64 (m, 2H), 1.12-0.98 (m, 2H), 0.77-0.73 (m, 2H).

6-[(1r,2r)-1,2-difluorocyclopropyl]-2-[(4-methoxyphenyl)methyl]spiro[3H-isoquinoline-4,1′-cyclopropane]-1-one (Int. 54): ¹H NMR (400 MHz, CDCl₃): δ 8.18 (d, J=7.6 Hz, 1H), 7.25 (d, J=8.6 Hz, 2H), 6.94 (d, J=8.1 Hz, 1H), 6.87 (d, J=8.7 Hz, 2H), 6.82 (d, J=1.5 Hz, 1H), 4.72 (s, 2H), 4.67-4.45 (m, 1H), 3.81 (s, 3H), 3.22 (s, 2H), 1.99-1.79 (m, 1H), 1.63-1.50 (m, 1H), 1.08-1.01 (m, 2H), 0.81-0.68 (m, 2H).

### Intermediate 55

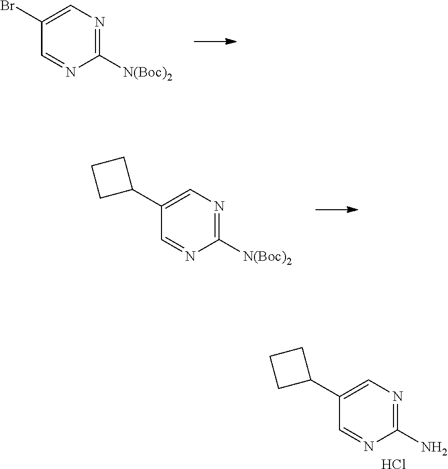

tert-butyl N-tert-butoxycarbonyl-N-(5-cyclobutylpyrimidin-2-yl)carbamate: A solution of tert-butyl N-(5-bromopyrimidin-2-yl)-N-tert-butoxycarbonyl-carbamate (500 mg, 1.34 mmol), potassium cyclobutyltrifluoroboranuide (325 mg, 2.00 mmol), Na₂CO₃ (283 mg, 2.67 mmol), 4,4′-di-tert-butyl-2,2′-bipyridine (18 mg, 0.07 mmol), bis[3,5-difluoro-2-[5-(trifluoromethyl)-2-pyridyl]phenyl]iridium(1+);2-(2-pyridyl)pyridine hexafluorophosphate (13.5 mg, 0.013 mmol), and dichloro(1,2-dimethoxyethane)nickel (15 mg, 0.067 mmol) in DMA (10 mL) was stirred for 16 h under a 34 W blue light. The reaction mixture was poured into aq. sat. NH₄Cl (30 mL) and extracted with EtOAc (3×10 mL). The combined organic layers were washed with brine (10 mL), dried over anhydrous Na₂SO₄, filtered, concentrated under reduced pressure, and purified by silica gel column chromatography. LCMS: m/z=350.2 [M+H]⁺.

5-cyclobutylpyrimidin-2-amine hydrochloride 9 (Int. 55): A solution of tert-butyl N-tert-butoxycarbonyl-N-(5-cyclobutylpyrimidin-2-yl)carbamate (100 mg, 0.29 mmol) in HCl (4M in EtOAc, 10 mL) was stirred for 16 h. The reaction mixture was concentrated under reduced pressure to provide a residue that was used directly. LCMS: m/z=150.2 [M+H]⁺.

### Intermediate 56

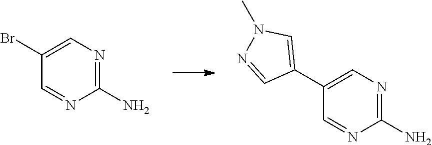

5-(1-methylpyrazol-4-yl)pyrimidin-2-amine: To a solution of 5-bromopyrimidin-2-amine (500 mg, 2.87 mmol) and 1-methyl-4-(4,4,5,5-tetramethyl-1,3,2-dioxaborolan-2-yl)-1H-pyrazole (658 mg, 3.16 mmol) in 1,4-dioxane (5 mL) and H₂O (1 mL) were added Pd(dppf)Cl₂ (421 mg, 0.57 mmol) and K₂CO₃ (993 mg, 7.18 mmol). The reaction mixture was stirred at 80° C. for 3 h. The reaction mixture was poured into H₂O (10 mL) and extracted with DCM (4×10 mL). The combined organic layers were washed with brine (10 mL), dried over anhydrous Na₂SO₄, filtered, and concentrated under reduced pressure. The residue was purified by preparative silica gel thin-layer chromatography. LCMS: m/z=176.1 [M+H]⁺.

### Intermediate 57

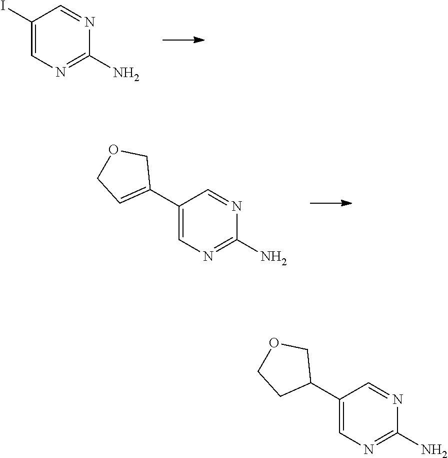 [SMILES: Nc1ncc(I)cn1 ; Nc1ncc(C2=CCOC2)cn1 ; Nc1ncc(C2CCOC2)cn1]

5-(2,5-dihydrofuran-3-yl)pyrimidin-2-amine: To a solution of 5-iodopyrimidin-2-amine (500 mg, 2.26 mmol) and 2-(2,5-dihydrofuran-3-yl)-4,4,5,5-tetramethyl-1,3,2-dioxaborolane (444 mg, 2.26 mmol) in 1,4-dioxane (5 mL) and H₂O (0.5 mL) were added K₂CO₃ (625 mg, 4.52 mmol) and Pd(dppf)Cl₂ (82 mg, 0.11 mmol). The mixture was stirred at 90° C. for 16 h. The reaction mixture was filtered then quenched by addition of H₂O (10 mL) at 20° C. The mixture was extracted with DCM:MeOH (10:1, 6×5 mL). The combined organic layers were washed with brine (15 mL), dried over anhydrous Na₂SO₄, filtered and concentrated under reduced pressure. The residue was purified by silica gel column chromatography. LCMS: m/z=164.0 [M+H]⁺.

5-tetrahydrofuran-3-ylpyrimidin-2-amine (Int. 57): To a solution of 5-(2,5-dihydrofuran-3-yl)pyrimidin-2-amine (230 mg, 1.41 mmol) in MeOH (4.5 mL), THF (4.5 mL) and EtOAc (4.5 mL) was added Pd/C (200 mg, 10% purity) under H₂. The mixture was stirred at 20° C. for 5 h. The reaction mixture was filtered and concentrated under reduced pressure to provide a residue that was used directly. LCMS: m/z=166.0 [M+H]⁺.

### Intermediate 58

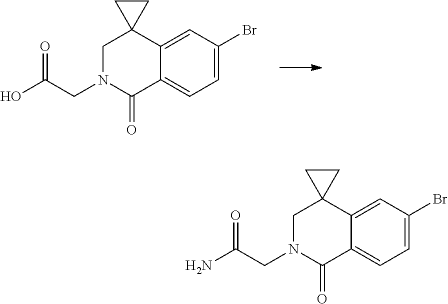 [SMILES: O=C(O)CN1CC2(CC2)c2cc(Br)ccc2C1=O ; NC(=O)CN1CC2(CC2)c2cc(Br)ccc2C1=O]

2-(6-bromo-1-oxo-spiro[3H-isoquinoline-4,1′-cyclopropane]-2-yl)acetamide: To a solution of 2-(6-bromo-1-oxo-spiro[3H-isoquinoline-4,1′-cyclopropane]-2-yl)acetic acid (2 g, 6.45 mmol, Int. 3), NH₄Cl (690 mg, 12.90 mmol), HOBt (1.05 g, 7.74 mmol) in DCM (20 mL) were added EDCI (1.48 g, 7.74 mmol) and DIPEA (1.67 g, 12.90 mmol). The mixture was stirred at 20° C. for 16 h. The mixture was added to H₂O (30 mL) and extracted with DCM (3×20 mL). The combined organics were washed with brine (20 mL), dried over anhydrous Na₂SO₄, filtered and concentrated under reduced pressure. The crude product were triturated with MTBE at 25° C. for 30 min to provide a residue that was used directly. LCMS: m/z=309.0, 311.0 [M+H]⁺.

### Intermediate 59

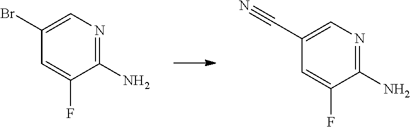 [SMILES: Nc1ncc(Br)cc1F ; N#Cc1cnc(N)c(F)c1]

4-amino-3-fluoro-benzonitrile: To a solution of 5-bromo-3-fluoropyridin-2-amine (300 mg, 1.57 mmol), K₄[Fe(CN)₆] (231 mg, 0.63 mmol), and Pd(PPh₃)₄ (91 mg, 0.08 mmol) in t-BuOH (3 mL) and water (3 mL) was added DBU (60 mg, 0.4 mmol). The reaction mixture was stirred at 85° C. for 12 h and then at 120° C. for 3 h. The reaction mixture was poured into H₂O (5 mL) and extracted with EtOAc (3×5 mL). The combined organic layers were washed with brine (5 mL), dried over anhydrous Na₂SO₄, filtered, and concentrated under reduced pressure. The residue was purified by silica gel column chromatography. ¹H NMR (400 MHz, CDCl₃): δ 8.19 (s, 1H), 7.41 (dd, J=2.0, 10.4 Hz, 1H), 5.19 (br s, 2H).

### Intermediate 60

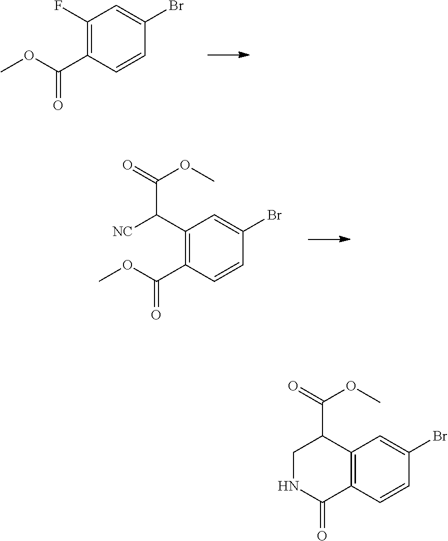 [SMILES: COC(=O)c1ccc(Br)cc1F ; COC(=O)c1ccc(Br)cc1C(C#N)C(=O)OC ; COC(=O)C1CNC(=O)c2ccc(Br)cc21]

methyl 4-bromo-2-(1-cyano-2-methoxy-2-oxoethyl)benzoate: To a solution of methyl 4-bromo-2-fluorobenzoate (50 g, 214 mmol) and Cs₂CO₃ (140 g, 429 mmol) in DMF (800 mL) was added methyl 2-cyanoacetate (25.5 g, 257 mmol). The mixture was stirred at 90° C. for 16 h. The reaction mixture was poured into H₂O (1.5 L) at 0° C., and extracted with EtOAc (3×500 mL). The combined organic layers were washed with aq. sat. NaHCO₃ (3×200 mL). The combined organic layers were washed with brine (200 mL), dried over anhydrous Na₂SO₄, filtered and concentrated under reduced pressure. The residue was purified by silica gel column chromatography.

methyl 6-bromo-1-oxo-3,4-dihydro-2H-isoquinoline-4-carboxylate (Int. 60): To a solution of methyl 4-bromo-2-(1-cyano-2-methoxy-2-oxoethyl)benzoate (10 g, 32 mmol) in MeOH (420 mL) and H₂O (4.2 mL) at 0° C. was added CoCl₂ (4.16 g, 32 mmol) and NaBH₄ (4.85 g, 128 mmol). The mixture was stirred at 0° C. for 3 h. The reaction mixture was quenched with HCl (3M) to pH=3 and concentrated under reduced pressure. The mixture was extracted with EtOAc (3×300 mL). The combined organic layers were washed with brine (400 mL) dried over anhydrous Na₂SO₄, filtered and concentrated under reduced pressure. The residue was purified by silica gel column chromatography. LCMS: m/z=284.0, 286.0 [M+H]⁺.

### Intermediate 61

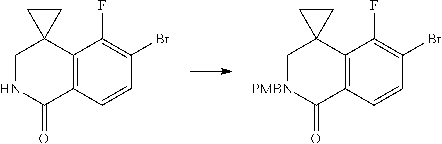

To a solution of 6-bromo-5-fluoro-spiro[2,3-dihydroisoquinoline-4,1′-cyclopropane]-1-one (5.3 g, 19.6 mmol) and 1-(chloromethyl)-4-methoxy-benzene (3.69 g, 23.6 mmol) in THF (50 mL) at 0° C. was added NaH (1.57 g, 39.2 mmol, 60% purity). The mixture was stirred at 50° C. for 20 h. The reaction mixture was poured into H₂O (50 mL) and extracted with EtOAc (3×20 mL). The combined organic layers were washed with brine (20 mL), dried over anhydrous Na₂SO₄, filtered and concentrated under reduced pressure. The residue was purified by silica gel column chromatography. LCMS: m/z=390.0, 392.0 [M+H]⁺.

### Intermediate 62 and 63

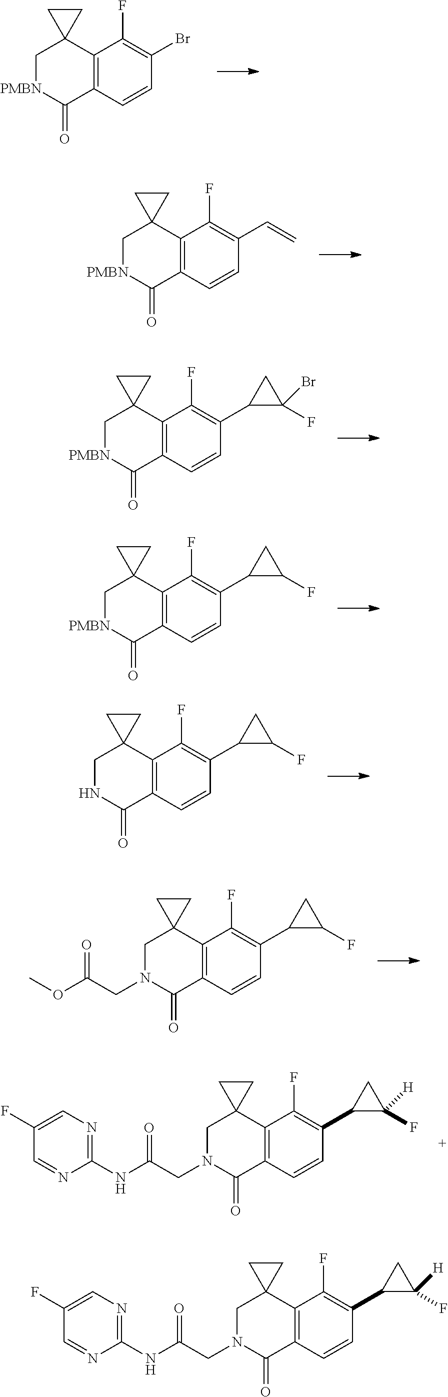

5′-fluoro-2′-(4-methoxybenzyl)-6′-vinyl-2′,3′-dihydro-1′H-spiro[cyclopropane-1,4′-isoquinolin]-1′-one: To a solution of 6′-bromo-5′-fluoro-2′-(4-methoxybenzyl)-2′,3′-dihydro-1′H-spiro[cyclopropane-1,4′-isoquinolin]-1′-one (4.0 g, 10.2 mmol, Int. 61) and potassium vinyltrifluoroborate (1.37 g, 10.2 mmol) in 1,4-dioxane (40 mL) and H₂O (10 mL) were added Pd(dppf)Cl₂ (750 mg, 1.02 mmol) and CsF (3.11 g, 20.5 mmol). The mixture was stirred at 100° C. for 12 h. The reaction mixture was poured into H₂O (80 mL) and extracted with EtOAc (3×40 mL). The combined organic layers were dried over anhydrous Na₂SO₄, filtered, and concentrated under reduced pressure. The residue was purified by silica gel column chromatography. LCMS: m/z=338.2 [M+H]⁺.

6′-(2-bromo-2-fluorocyclopropyl)-5′-fluoro-2′-(4-methoxybenzyl)-2′,3′-dihydro-1′H-spiro[cyclopropane-1,4′-isoquinolin]-1′-one: To a solution of 5′-fluoro-2′-(4-methoxybenzyl)-6′-vinyl-2′,3′-dihydro-1′H-spiro[cyclopropane-1,4′-isoquinolin]-1′-one (2.7 g, 8.0 mmol) in DCM (15 mL) at 0° C. were added aq. NaOH (28.8 g, 360 mmol, 50% (w/w)), dibromo(fluoro)methane (7.68 g, 40.0 mmol), and benzyltriethylammonium chloride (183 mg, 0.80 mmol). The mixture was stirred at 0° C. for 0.5 h, then stirred at 20° C. for 12 h. The reaction mixture was poured into ice-cold H₂O (40 mL) and extracted with EtOAc (2×20 mL). The combined organic layers were dried over anhydrous Na₂SO₄, filtered, and concentrated under reduced pressure. The residue was purified by silica gel column chromatography. LCMS: m/z=448.0, 450.1 [M+H]⁺.

5′-fluoro-6′-(2-fluorocyclopropyl)-2′-(4-methoxybenzyl)-2′,3′-dihydro-1′H-spiro[cyclopropane-1,4′-isoquinolin]-1′-one: To a solution of 6′-(2-bromo-2-fluorocyclopropyl)-5′-fluoro-2′-(4-methoxybenzyl)-2′,3′-dihydro-1′H-spiro[cyclopropane-1,4′-isoquinolin]-1′-one (2.4 g, 5.35 mmol) in tributylstannane (26.4 g, 90.7 mmol) at 20° C. was added AIBN (88 mg, 0.54 mmol). The mixture was stirred at 80° C. for 3 h. The reaction mixture was poured into aq. sat. KF (40 mL), stirred for 1 h at 20° C., and extracted with EtOAc (4×30 mL). The combined organic layers were washed with brine (10 mL), dried over anhydrous Na₂SO₄, filtered, and concentrated under reduced pressure. The residue was purified by silica gel column chromatography. LCMS: m/z=370.1 [M+H]⁺.

5′-fluoro-6′-(2-fluorocyclopropyl)-2′,3′-dihydro-1′H-spiro[cyclopropane-1,4′-isoquinolin]-1′-one: 5′-fluoro-6′-(2-fluorocyclopropyl)-2′-(4-methoxybenzyl)-2′,3′-dihydro-1′H-spiro[cyclopropane-1,4′-isoquinolin]-1′-one (1.4 g, 3.79 mmol) was added to TFA (15 mL) at 20° C. and the mixture was stirred at 60° C. for 5 h. The reaction mixture was poured into H₂O (10 mL) and the aqueous layer was adjusted to pH=8 with aq. sat. NaHCO₃. The mixture was extracted with EtOAc (3×20 mL). The combined organic layers were dried over anhydrous Na₂SO₄, filtered, and concentrated under reduced pressure. The residue was purified by silica gel column chromatography. LCMS: m/z=250.1 [M+H]⁺.

methyl 2-(5′-fluoro-6′-(2-fluorocyclopropyl)-1′-oxo-1′H-spiro[cyclopropane-1,4′-isoquinolin]-2′(3′H)-yl)acetate: To a solution of 5′-fluoro-6′-(2-fluorocyclopropyl)-2′,3′-dihydro-1′H-spiro[cyclopropane-1,4′-isoquinolin]-1′-one (200 mg, 0.80 mmol) in DMF (3.0 mL) at 0° C. was added NaH (48 mg, 1.20 mmol, 60% purity). The mixture was stirred for 15 min and methyl 2-bromoacetate (245 mg, 1.60 mmol) was added. The mixture was stirred at 25° C. for 2 h. The reaction mixture was quenched by addition aq. sat. NH₄Cl (10 mL) and extracted with EtOAc (3×10 mL). The combined organic layers were dried over anhydrous Na₂SO₄, filtered, and concentrated under reduced pressure. The residue was purified by silica gel column chromatography. LCMS: m/z=322.2 [M+H]⁺.

2-(5′-fluoro-6′-((1r,2r)-2-fluorocyclopropyl)-1′-oxo-1′H-spiro[cyclopropane-1,4′-isoquinolin]-2′(3′H)-yl)-N-(5-fluoropyrimidin-2-yl)acetamide and 2-(5′-fluoro-6′-((1s,2r)-2-fluorocyclopropyl)-1′-oxo-1′H-spiro[cyclopropane-1,4′-isoquinolin]-2′(3′H)-yl)-N-(5-fluoropyrimidin-2-yl)acetamide: To a solution of methyl 2-(5′-fluoro-6′-(2-fluorocyclopropyl)-1′-oxo-1′H-spiro[cyclopropane-1,4′-isoquinolin]-2′(3′H)-yl)acetate (100 mg, 0.31 mmol) and 5-fluoropyrimidin-2-amine (70 mg, 0.62 mmol) in DCE (3.0 mL) at 25° C. was added AlMe₃ (1 M in n-heptane, 0.62 mmol). The mixture was stirred at 90° C. for 3 h. The mixture was diluted with H₂O (30 mL) and extracted with DCM (3×10 mL). The combined organic layers were dried over anhydrous Na₂SO₄, filtered, and concentrated under reduced pressure. The residue was purified by reverse-phase preparative HPLC to provide:

2-(5′-fluoro-6′-((1r,2r)-2-fluorocyclopropyl)-1′-oxo-1′H-spiro[cyclopropane-1,4′-isoquinolin]-2′(3′H)-yl)-N-(5-fluoropyrimidin-2-yl)acetamide (Int. 62)

LCMS: m/z=403.0 [M+H]⁺. ¹H NMR (400 MHz, CDCl₃): δ 9.06 (br s, 1H), 8.48 (s, 2H), 7.94 (d, J=8.0 Hz, 1H), 7.15 (t, J=7.2 Hz, 1H), 5.00-4.73 (m, 1H), 4.60-4.47 (m, 2H), 3.54-3.36 (m, 2H), 2.26-2.06 (m, 1H), 1.64-1.58 (m, 2H), 1.41-1.23 (m, 2H), 1.06-0.91 (m, 2H).

2-(5′-fluoro-6′-((1s,2r)-2-fluorocyclopropyl)-1′-oxo-1′H-spiro[cyclopropane-1,4′-isoquinolin]-2′(3′H)-yl)-N-(5-fluoropyrimidin-2-yl)acetamide (Int. 63). LCMS: m/z=403.0 [M+H]⁺. ¹H NMR (400 MHz, CDCl₃): δ 9.06-8.89 (m, 1H), 8.48 (s, 2H), 7.90 (d, J=8.0 Hz, 1H), 6.80-6.68 (m, 1H), 4.79-4.59 (m, 1H), 4.55 (br s, 2H), 3.44 (s, 2H), 2.63-2.44 (m, 1H), 1.67-1.55 (m, 3H), 1.22-1.12 (m, 1H), 1.00 (s, 2H).

### Intermediate 64

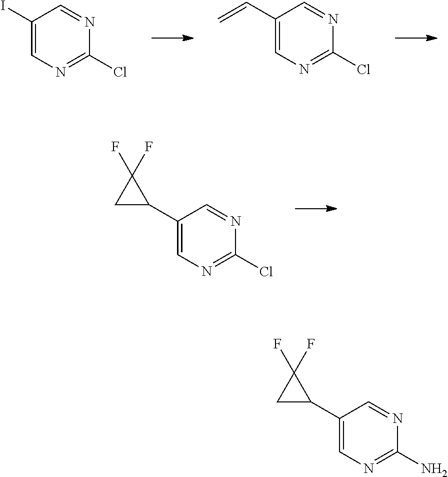

2-chloro-5-vinylpyrimidine: To a solution of 2-chloro-5-iodo-pyrimidine (2.0 g, 8.32 mmol) and potassium trifluorovinylborate (1.11 g, 8.32 mmol) in 1,4-dioxane (40 mL) at 15° C. were added CsF (2.53 g, 16.64 mmol) and Pd(dppf)Cl₂ (609 mg, 0.83 mmol). The mixture was heated to 80° C., and stirred for 5 h. The mixture was diluted with H₂O (60 mL) and extracted with EtOAc (3×20 mL). The combined organic layers were washed with brine (30 mL), dried over anhydrous Na₂SO₄, filtered and concentrated under reduced pressure. The residue was purified by silica gel column chromatography. LCMS: m/z=141.1, 143.0 [M+H]⁺.

2-chloro-5-(2,2-difluorocyclopropyl)pyrimidine: To a solution of 2-chloro-5-vinyl-pyrimidine (300 mg, 2.13 mmol) and NaI (96 mg, 0.64 mmol) in THF (3.0 mL) at 80° C. was added TMSCF₃ (1.52 g, 10.7 mmol). The mixture was stirred for 1 h at 80° C. The mixture was diluted with H₂O (10 mL) and extracted with EtOAc (3×3 mL). The combined organic layers were washed with brine (3 mL), dried over anhydrous Na₂SO₄, filtered, and concentrate under reduced pressure. The residue was purified by silica gel column chromatography. LCMS: m/z=191.0, 193.0 [M+H]⁺.

5-(2,2-difluorocyclopropyl)pyrimidin-2-amine: To a solution of 2-chloro-5-(2,2-difluorocyclopropyl)pyrimidine (30 mg, 0.16 mmol) in 1,4-dioxane (0.5 mL) at 20° C. was added NH₄OH (0.5 mL). The mixture was heated to 65° C., and stirred for 2 h. The mixture was concentrated under reduced pressure to provide a residue that was used directly. LCMS: m/z=172.1 [M+H]⁺.

### Intermediate 65

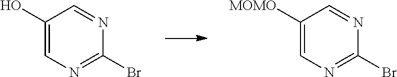

2-bromo-5-(methoxymethoxy)pyrimidine: To a solution of 2-bromopyrimidin-5-ol (5.0 g, 28.6 mmol) in THF (50 mL) at 0° C. were added Et₃N (3.47 g, 34.3 mmol) and MOMCl (2.5 g, 31.4 mmol). The reaction mixture was quenched by addition aq. HCl (1M, 50 mL) and extracted with EtOAc (3×20 mL). The combined organic layers were washed with aq. NaOH (1M, 15 mL) and brine (3×20 mL), dried over anhydrous Na₂SO₄, filtered, and concentrated under reduced pressure to provide a residue. The residue was purified by silica gel column chromatography. LCMS: m/z=219.0, 221.0 [M+H]⁺.

### Intermediate 66

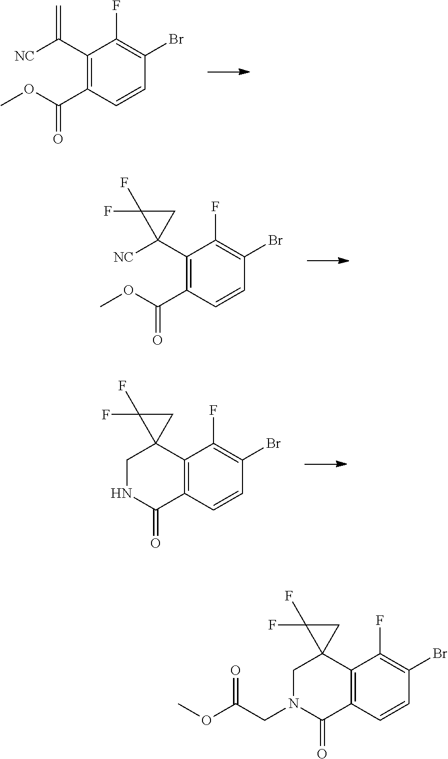 [SMILES: C=C(C#N)c1c(C(=O)OC)ccc(Br)c1F ; COC(=O)c1ccc(Br)c(F)c1C1(C#N)CC1(F)F ; O=C1NCC2(CC2(F)F)c2c1ccc(Br)c2F ; COC(=O)CN1CC2(CC2(F)F)c2c(ccc(Br)c2F)C1=O]

methyl 4-bromo-2-(1-cyano-2,2-difluorocyclopropyl)-3-fluorobenzoate: To a solution of methyl 4-bromo-2-(1-cyanovinyl)-3-fluorobenzoate (450 mg, 1.58 mmol, Int. 36) in 1,4-dioxane (2.0 mL) was added sodium 2-chloro-2,2-difluoroacetate (725 mg, 4.75 mmol). The mixture was stirred at 150° C. for 20 min under microwave irradiation. The reaction mixture was poured into H₂O (20 mL) and extracted with EtOAc (4×15 mL). The combined organic layers were washed with brine (2×10 mL), dried over anhydrous Na₂SO₄, filtered, concentrated under reduced pressure, and purified by reverse-phase preparative HPLC. LCMS: m/z=333.9, 335.9 [M+H]⁺.

6′-bromo-2,2,5′-trifluoro-2′,3′-dihydro-1′H-spiro[cyclopropane-1,4′-isoquinolin]-1′-one: To a mixture of methyl 4-bromo-2-(1-cyano-2,2-difluorocyclopropyl)-3-fluorobenzoate (600 mg, 1.80 mmol) and CoCl₂ (233 mg, 1.80 mmol) in MeOH (12 mL) and H₂O (1.2 mL) at −10° C. was added NaBH₄ (204 mg, 5.39 mmol). The mixture was stirred at 0° C. for 1 h and then −10° C. for a further 4 h. The reaction mixture was diluted with sat. aq. NH₄Cl(20 mL) and extracted with EtOAc (4×15 mL). The combined organic layers were washed with brine (2×10 mL), dried over anhydrous Na₂SO₄, filtered, concentrated under reduced pressure, and purified by silica gel column chromatography. LCMS: m/z=306.0, 308.0 [M+H]⁺.

methyl 2-(6′-bromo-2,2,5′-trifluoro-1′-oxo-1′H-spiro[cyclopropane-1,4′-isoquinolin]-2′(3′H)-yl)acetate: To a solution of methyl 2-bromoacetate (82 mg, 0.54 mmol) in DMF (1.5 mL) were added 6′-bromo-2,2,5′-trifluoro-2′,3′-dihydro-1′H-spiro[cyclopropane-1,4′-isoquinolin]-1′-one (110 mg, 0.36 mmol), Cs₂CO₃ (234 mg, 0.72 mmol), and NaI (27 mg, 0.18 mmol). The mixture was stirred at 20° C. for 2 h. The reaction mixture was cooled to 0° C., diluted with H₂O (10 mL), and extracted with EtOAc (4×5 mL). The combined organic layers were washed with brine (10 mL), dried over anhydrous Na₂SO₄, filtered, concentrated under reduced pressure, and purified by silica gel column chromatography. LCMS: m/z=378.0, 380.0 [M+H]⁺.

### Intermediate 67

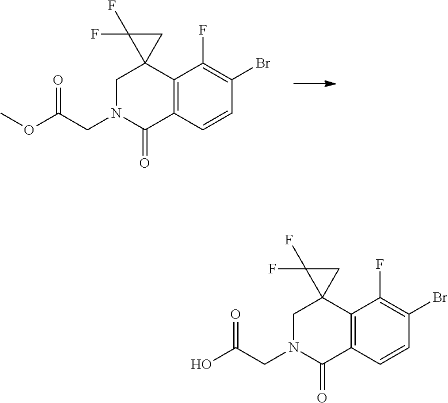 [SMILES: COC(=O)CN1CC2(CC2(F)F)c2c(ccc(Br)c2F)C1=O ; O=C(O)CN1CC2(CC2(F)F)c2c(ccc(Br)c2F)C1=O]

2-(6′-bromo-2,2,5′-trifluoro-1′-oxo-1′H-spiro[cyclopropane-1,4′-isoquinolin]-2′(3′H)-yl)acetic acid: To a solution of methyl 2-(6′-bromo-2,2,5′-trifluoro-1′-oxo-1′H-spiro[cyclopropane-1,4′-isoquinolin]-2′(3′H)-yl)acetate (115 mg, 0.30 mmol, Int. 66) in THF (3.0 mL) and H₂O (0.6 mL) was added LiOH·H₂O (26 mg, 0.61 mmol). The mixture was stirred at 20° C. for 1 h. The reaction mixture was poured into H₂O (10 mL) and washed with MTBE (3×5 mL). The aqueous layer was adjusted to pH=3 with aq. HCl (3 M) at 0° C., and extracted with EtOAc (3×10 mL). The combined organic layers were washed with brine (10 mL), dried over anhydrous Na₂SO₄, filtered, and concentrated under reduced pressure to provide a residue that was used directly. LCMS: m/z=361.9, 363.9 [M−H]⁻.

### Example 1

### 2-(6-bromo-4-ethyl-1-oxo-3,4-dihydroisoquinolin-2(1H)-yl)-N-(5-fluoropyrimidin-4-yl)acetamide (1)

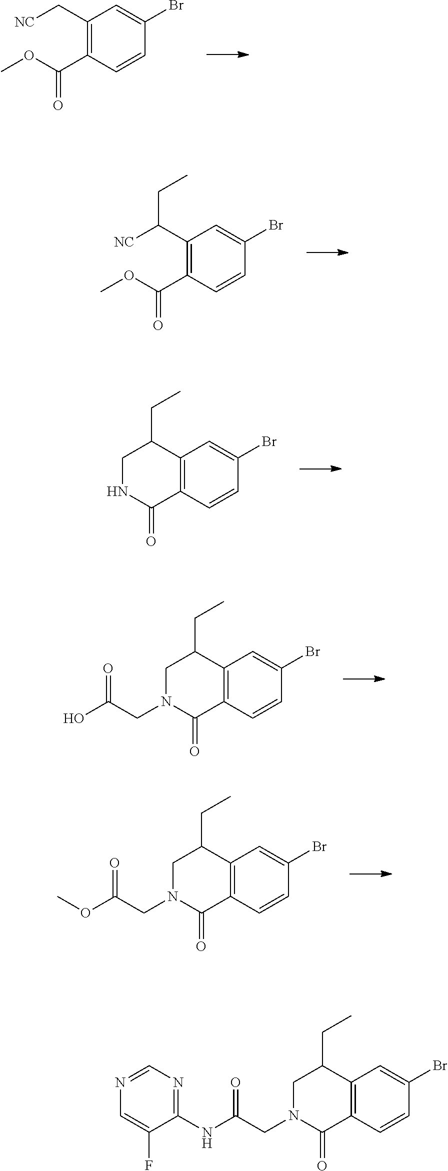 [SMILES: COC(=O)c1ccc(Br)cc1CC#N ; CCC(C#N)c1cc(Br)ccc1C(=O)OC ; CCC1CNC(=O)c2ccc(Br)cc21 ; CCC1CN(CC(=O)O)C(=O)c2ccc(Br)cc21 ; CCC1CN(CC(=O)OC)C(=O)c2ccc(Br)cc21 ; CCC1CN(CC(=O)Nc2ncncc2F)C(=O)c2ccc(Br)cc21]

Methyl 4-bromo-2-(1-cyanopropyl)benzoate: To a solution of methyl 4-bromo-2-(cyanomethyl)benzoate (500 mg, 1.97 mmol) in THF (8 mL) was added NaHMDS (2.16 mmol, 1 M in THF) dropwise at −78° C. The reaction mixture was stirred at −78° C. for 0.5 h before the addition of a solution of iodoethane (307 mg, 1.97 mmol) in THF (2 mL). The reaction mixture was stirred at −78° C. for a further 1 h and then stirred at 25° C. for 2 h. The reaction mixture was poured into 1 M aqueous HCl (20 mL) and extracted with EtOAc (3×8 mL). The combined organic layers were washed with brine (5 mL), dried over anhydrous Na₂SO₄, filtered, and concentrated under reduced pressure. The residue was purified by silica gel column chromatography. LCMS: m/z=282.0, 283.9 [M+H]⁺.

6-Bromo-4-ethyl-3,4-dihydroisoquinolin-1(2H)-one: To a mixture of methyl 4-bromo-2-(1-cyanopropyl)benzoate (200 mg, 0.71 mmol) and dichlorocobalt (184 mg, 1.42 mmol) in MeOH (4 mL) was added NaBH₄ (134 mg, 3.54 mmol) at 0° C. The reaction mixture was stirred at 50° C. for 6 h. The reaction mixture was poured into water (10 mL) and extracted with EtOAc (3×5 mL). The combined organic layers were washed with brine (5 mL), dried over anhydrous Na₂SO₄, filtered, and concentrated under reduced pressure. The residue was purified by silica gel column chromatography. LCMS: m/z=253.9, 255.9 [M+H]⁺.

2-(6-bromo-4-ethyl-1-oxo-3,4-dihydroisoquinolin-2(1H)-yl)acetic acid: To a solution of 6-bromo-4-ethyl-3,4-dihydroisoquinolin-1(2H)-one (120 mg, 0.47 mmol) in THF (3 mL) was added NaH (20 mg, 0.52 mmol, 60% purity) at 0° C. The reaction mixture was stirred at 0° C. for 0.5 h before the addition of methyl 2-bromoacetate (79 mg, 0.52 mmol). The reaction mixture was stirred at 25° C. for a further 2 h. The reaction mixture was poured into water (10 mL). The pH was adjusted to pH=3 using aqueous 3 N HCl and then the mixture was extracted with EtOAc (3×10 mL). The combined organic layers were dried over anhydrous Na₂SO₄, filtered, and concentrated under reduced pressure to provide a residue that was used directly. LCMS: m/z=311.9, 313.9 [M+H]⁺.

Methyl 2-(6-bromo-4-ethyl-1-oxo-3,4-dihydroisoquinolin-2(1H)-yl)acetate: To a solution of 2-(6-bromo-4-ethyl-1-oxo-3,4-dihydroisoquinolin-2(1H)-yl)acetic acid (130 mg, 0.42 mmol) in MeOH (1 mL) was added SOCl₂ (99 mg, 0.83 mmol) at 0° C. The reaction mixture was stirred at 60° C. for 1 h. The reaction mixture was concentrated under reduced pressure. The residue was resuspended in MTBE (5 mL) and adjusted to pH=7 with sat. aqueous NaHCO₃. The layers were separated and the aqueous phase was extracted with MTBE (2×5 mL). The combined organic layers were washed with brine (5 mL), dried over anhydrous Na₂SO₄, filtered, and concentrated under reduced pressure to provide a residue that was used directly.

2-(6-bromo-4-ethyl-1-oxo-3,4-dihydroisoquinolin-2(1H)-yl)-N-(5-fluoropyrimidin-4-yl)acetamide: To a solution of methyl 2-(6-bromo-4-ethyl-1-oxo-3,4-dihydroisoquinolin-2(1H)-yl)acetate (70 mg, 0.21 mmol) in toluene (1 mL) and THF (1 mL) were added 5-fluoropyrimidin-4-amine (48 mg, 0.43 mmol) and AlMe₃ (0.64 mmol, 2 M in toluene) at 25° C. The reaction mixture was stirred at 90° C. for 4 h. The reaction mixture was poured into water (5 mL) and filtered. The filtrate was extracted with EtOAc (4×5 mL). The combined organic layers were dried over anhydrous Na₂SO₄, filtered, and concentrated under reduced pressure. The crude residue was purified by reverse-phase HPLC. LCMS: m/z=407.0, 409.0 [M+H]⁺. ¹H NMR (400 MHz, CDCl₃): δ 8.82-8.69 (m, 1H), 8.49 (d, J=2.4 Hz, 1H). 7.97 (d, J=8.4 Hz, 1H), 7.52 (dd, J=1.6, 8.4 Hz, 1H), 7.39 (d, J=1.6 Hz, 1H), 4.74-4.48 (m, 2H), 3.99 (dd, J=4.4, 12.4 Hz, 1H), 3.54 (dd, J=3.6, 12.4 Hz, 1H), 2.82 (m, 1H), 1.81-1.71 (m, 2H), 1.00 (t, J=7.6 Hz, 3H).

### Example 2

### 2-(6-bromo-1-oxo-spiro[3H-isoquinoline-4,1′-cyclopropane]-2-yl)-N-(5-fluoropyrimidin-2-yl)acetamide (2)

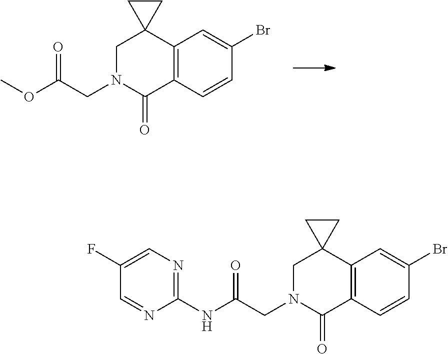 [SMILES: COC(=O)CN1CC2(CC2)c2cc(Br)ccc2C1=O ; O=C(CN1CC2(CC2)c2cc(Br)ccc2C1=O)Nc1ncc(F)cn1]

To a mixture of 5-fluoropyrimidin-2-amine (49 mg, 0.43 mmol) and methyl 2-(6-bromo-1-oxo-spiro[3H-isoquinoline-4,1′-cyclopropane]-2-yl)acetate (70 mg, 0.22 mmol) in toluene (2 mL) and THF (2 mL) was added dropwise AlMe₃ (0.30 mL, 2 M in toluene) at 20° C. The reaction mixture was stirred for 3 h at 90° C. The reaction mixture was poured into ice-cold H₂O (10 mL) and extracted with EtOAc (4×5 mL). The combined organic layers were washed with brine (2×10 mL), dried over anhydrous Na₂SO₄, filtered, and concentrated under reduced pressure. The crude residue was purified by reverse-phase HPLC. LCMS: m/z=405.0, 407.0 [M+H]⁺. H NMR (400 MHz, CDCl₃): δ 9.06 (br s, 1H), 8.49 (s, 2H), 8.02 (d, =8.4 Hz, 1H), 7.46 (dd, J=1.6, 8.4 Hz, TH), 7.01 (d, =1.6 Hz, 1H), 4.64-4.49 (m, 2H), 3.54 (s, 2H), 1.17-1.11 (, 2H), 1.10-1.05 (, 2H).

The following compounds as shown in Table 1 were, or can be, made via similar procedures as those described above.

| Ex. | Name | NMR | LCMS |
|---|---|---|---|
| 3 | 2-(6-bromo-1- | ¹H NMR (400 MHz, | m/z = 411.9, |
| | oxo-spiro[3H- | CDCl₃): δ 9.27 (br s, | 414.0 |
| | isoquinoline-4,1′- | 1H), 8.86 (s, 2H), 8.01 | [M + H]⁺ |
| | cyclopropane]- | (d, J = 8.4 Hz, 1H), | |
| | 2-yl)-N-(5- | 7.47 (dd, J = 1.6, 8.4 Hz, | |
| | cyanopyrimidin-2- | 1H), 7.01 (d, J = 1.6 | |
| | yl)acetamide | Hz, 1H), 4.62 (s, 2H), | |
| | | 3.54 (s, 2H), 1.18-1.12 | |
| | | (m, 2H), 1.11-1.05 (m, 2H) | |
| 4 | 2-(6-bromo-1- | ¹H NMR (400 MHz, | m/z = |
| | oxo-spiro[3H- | CDCl₃): δ 8.96 (br s, | 421.1, |
| | isoquinoline-4,1′- | 1H), 8.55 (s, 2H), 8.02 | 423.0 |
| | cyclopropane]- | (d, J = 8.4 Hz, 1H), | [M + H]⁺ |
| | 2-yl)-N-(5- | 7.46 (dd, J = 8.4, 1.6 Hz, | |
| | chloropyrimidin-2- | 1H), 7.01 (d, J = 1.6 | |
| | yl)acetamide | Hz, 1H), 4.60 (s, 2H), | |
| | | 3.53 (s, 2H), 1.16-1.11 | |
| | | (m, 2H), 1.10-1.05 (m, 2H) | |
| 5 | 2-(6-bromo-1- | ¹H NMR (400 MHz, | m/z = 387.1, |
| | oxo-spiro[3H- | CDCl₃): δ 9.16 (br s, | 389.1 |
| | isoquinoline-4,1′- | 1H), 8.62 (d, J = 4.8 Hz, | [M + H]⁺ |
| | cyclopropane]- | 2H), 8.01 (d, J = 8.4 | |
| | 2-yl)-N- | Hz, 1H), 7.45 (dd, J = 8.4, | |
| | pyrimidin-2- | 1.8 Hz, 1H), 7.03 | |
| | yl-acetamide | (t, J = 4.8 Hz, 1H), 7.00 | |
| | | (d, J = 1.6 Hz, 1H), | |
| | | 4.71 (br s, 2H), 3.54 | |
| | | (s, 2H), 1.16-1.11 (m, | |
| | | 2H), 1.10-1.05 (m, 2H) | |
| 6 | 2-(6-bromo-1- | ¹H NMR (400 MHz, | m/z = 404.9, |
| | oxospiro[3H- | DMSO-d₆): δ 8.77 (dd, J = | 406.9 |
| | isoquinoline-4,1′- | 2.8, 10.8 Hz, 2H), 7.81 | [M + H]⁺ |
| | cyclopropane]- | (d, J = 8.0 Hz, 1H), | |
| | 2-yl)-N-(5- | 7.52 (dd, J = 2.0, 8.4 Hz, | |
| | fluoropyrimidin-4- | 1H), 7.26 (d, J = 2.0 | |
| | yl)acetamide | Hz, 1H), 4.50 (s, 2H), | |
| | | 3.50 (s, 2H), 1.19-1.11 | |
| | | (m, 2H), 1.10-0.99 (m, 2H) | |
| 7 | 2-(6-bromo-1- | ¹H NMR (400 MHz, | m/z = 429.4, |
| | oxospiro[3H- | DMSO-d₆): δ 11.02- | 431.4 |
| | isoquinoline-4,1′- | 10.98 (m, 1H), 8.73 (dd, | [M + H]⁺ |
| | cyclopropane]- | J = 1.8, 0.6 Hz, 1H), | |
| | 2-yl)-N-(5- | 8.43 (dd, J = 10.3, 1.8 | |
| | cyano-3- | Hz, 1H), 7.82 (d, J = | |
| | fluoropyridin-2- | 8.3 Hz, 1H), 7.53 (dd, | |
| | yl)acetamide | J = 8.3, 1.9 Hz, 1H), | |
| | | 7.27 (d, J = 1.9 Hz, 1H), | |
| | | 4.49 (s, 2H), 3.51 (s, | |
| | | 2H), 1.18-1.15 (m, 2H), | |
| | | 1.07-1.04 (m, 2H) | |
| 8 | 2-(6-bromo-1- | ¹H NMR (400 MHz, | m/z = 438.3, |
| | oxospiro[3H- | DMSO-d₆): δ 10.65- | 440.3 |
| | isoquinoline-4,1′- | 10.64 (m, 1H), 8.37-8.36 | [M + H]⁺ |
| | cyclopropane]- | (m, 1H), 8.15 (dd, J = | |
| | 2-yl)-N-(5- | 9.7, 2.2 Hz, 1H), 7.82 | |
| | chloro-3- | (d, J = 8.4 Hz, 1H), | |
| | fluoropyridin-2- | 7.53 (dd, J = 8.3, 1.9 Hz, | |
| | yl)acetamide | 1H), 7.26 (d, J = 1.9 | |
| | | Hz, 1H), 4.43 (s, 2H), | |
| | | 3.51 (s, 2H), 1.16-1.14 | |
| | | (m, 2H), 1.07-1.04 (m, 2H) | |
| 9 | 2-(6-bromo-1- | ¹H NMR (400 MHz, | m/z = 472.3, |
| | oxospiro[3H- | DMSO-d₆): δ 10.94 (t, J = | 474.3 |
| | isoquinoline-4,1′- | 0.5 Hz, 1H), 8.68 (t, | [M + H]⁺ |
| | cyclopropane]- | J = 0.6 Hz, 1H), 8.37- | |
| | 2-yl)-N-[3- | 8.34 (m, 1H), 7.82 (d, | |
| | fluoro-5- | J = 8.3 Hz, 1H), 7.53 | |
| | (trifluoro- | (dd, J = 8.3, 1.9 Hz, 1H), | |
| | methyl)pyridin-2- | 7.27 (d, J = 1.9 Hz, | |
| | yl]acetamide | 1H), 4.49 (s, 2H), 3.52 | |
| | | (s, 2H), 1.18-1.15 (m, | |
| | | 2H), 1.07-1.04 (m, 2H) | |
| 10 | 2-(6-bromo-1- | ¹H NMR (400 MHz, | m/z = 421.3, |
| | oxospiro[3H- | DMSO-d₆): δ 11.56 (d, J = | 423.3 |
| | isoquinoline-4,1′- | 0.3 Hz, 1H), 8.36 (d, | [M + H]⁺ |
| | cyclopropane]- | J = 9.4 Hz, 1H), 7.89 | |
| | 2-yl)-N-(6- | (d, J = 9.4 Hz, 1H), | |
| | chloropyridazin-3- | 7.81 (d, J = 8.3 Hz, 1H), | |
| | yl)acetamide | 7.53 (dd, J = 8.3, 1.9 | |
| | | Hz, 1H), 7.27 (d, J = 1.9 | |
| | | Hz, 1H), 4.45 (s, 2H), | |
| | | 3.53 (s, 2H), 1.19-1.16 | |
| | | (m, 2H), 1.07-1.04 (m, 2H) | |
| 11 | 2-(6-bromo-1- | ¹H NMR (400 MHz, | m/z = 404.2, |
| | oxospiro[3H- | DMSO-d₆): δ 10.48 (s, | 406.2 |
| | isoquinoline-4,1′- | 1H), 8.55 (d, J = 2.9 Hz, | [M + H]⁺ |
| | cyclopropane]- | 1H), 8.31 (d, J = 5.4 | |
| | 2-yl)-N-(3- | Hz, 1H), 8.18 (dd, J = 6.9, | |
| | fluoropyridin- | 5.4 Hz, 1H), 7.82 | |
| | 4-yl)acetamide | (d, J = 8.3 Hz, 1H), 7.53 | |
| | | (dd, J = 8.3, 1.9 Hz, | |
| | | 1H), 7.27 (d, J = 1.9 | |
| | | Hz, 1H), 4.46 (s, 2H), | |
| | | 3.52 (s, 2H), 1.19-1.16 | |
| | | (m, 2H), 1.08-1.05 (m, | |
| | | 2H) | |
| 12 | 2-(6-bromo-4- | ¹H NMR (400 MHz, | m/z = 422.4, |
| | isopropyl-1- | DMSO-d₆): δ 10.54 (s, | 424.4 |
| | oxo-2- | 1H), 8.36-8.36 (m, | [M + H]⁺ |
| | isoquinolyl)-N-(3,5- | 1H), 8.03 (ddd, J = 9.9, | |
| | difluoro-2- | 8.5, 2.6 Hz, 1H), 7.82 | |
| | pyridyl)acetamide | (d, J = 8.3 Hz, 1H), | |
| | | 7.53 (dd, J = 8.3, 1.9 | |
| | | Hz, 1H), 7.26 (d, J = 1.9 | |
| | | Hz, 1H), 4.41 (s, 2H), | |
| | | 3.51 (s, 2H), 1.17-1.14 | |
| | | (m, 2H), 1.07-1.04 (m, 2H) | |

### Example 13

### 2-(6-bromo-1-oxospiro[3H-isoquinoline-4,1′-cyclopropane]-2-yl)-N-[3-(trifluoromethyl)-1-bicyclo[1.1.1]pentanyl]acetamide (13)

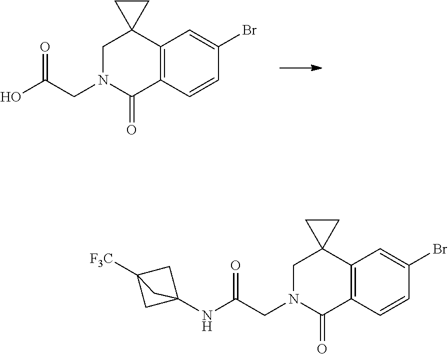 [SMILES: O=C(O)CN1CC2(CC2)c2cc(Br)ccc2C1=O ; O=C(CN1CC2(CC2)c2cc(Br)ccc2C1=O)NC12CC(C(F)(F)F)(C1)C2]

To a solution of 2-(6-bromo-1-oxo-spiro[3H-isoquinoline-4,1-cyclopropane]-2-yl)acetic acid (35 mg, 0.11 mmol) in DMF (1.4 mL) were added 3-(trifluoromethyl)bicyclo[1.1.1]pentan-1-amine HCl salt (29 mg, 0.16 mmol), DIPEA (43 mg, 0.34 mmol), and T3P (93 mg, 0.15 mmol, 50% in DMF). The reaction mixture was stirred at 25° C. for 1 h. The reaction mixture was purified directly by reverse-phase HPLC. LCMS: m/z=443.3, 445.4 [M+H]⁺. ¹H NMR (400 MHz, DMSO-d₆): δ 8.83 (s, 1H), 7.80 (d, J=8.3 Hz, 1H), 7.52 (dd, J=8.3, 1.9 Hz, 1H), 7.24 (d, J=1.9 Hz, 1H), 4.08 (s, 2H), 3.44 (s, 2H), 2.25-2.20 (m, 6H), 1.15-1.12 (m, 2H), 1.04-1.01 (m, 2H).

The following compounds as shown in Table 1 were, or can be, made via similar procedures as those described above.

| Ex. | Name | NMR | LCMS |
|---|---|---|---|
| 14 | 2-(6-bromo-1- | ¹H NMR (400 MHz, | m/z = |
| | oxospiro[3H- | DMSO-d₆): δ 8.83 | 400.4, |
| | isoquinoline-4,1′- | (s, 1H), 7.80 (d, J = | 402.3 |
| | cyclopropane]-2-yl)- | 8.3 Hz, 1H), 7.51 | [M + H]⁺ |
| | N-(3-cyano-1- | (dd, J = 8.3, 1.9 Hz, | |
| | bicyclo[1.1.1]pen- | 1H), 7.24 (d, J = 1.9 | |
| | tanyl)acetamide | Hz, 1H), 4.05 (s, 2H), | |
| | | 3.43 (s, 2H), 2.49- | |
| | | 2.43 (m, 6H), 1.15-1.11 | |
| | | (m, 2H), 1.03- | |
| | | 1.00 (m, 2H) | |
| 15 | 2-(6-bromo-1- | ¹H NMR (400 MHz, | m/z = |
| | oxospiro[3H- | DMSO-d₆): δ 8.02- | 427.5, |
| | isoquinoline-4,1′- | 8.00 (m, 1H), 7.81 (d, | 429.5 |
| | cyclopropane]-2-yl)- | J = 8.3 Hz, 1H), | [M + H]⁺ |
| | N-(4,4-difluoro- | 7.52 (dd, J = 8.3, 1.9 | |
| | cyclohexyl)acetamide | Hz, 1H), 7.24 (d, J = | |
| | | 1.9 Hz, 1H), 4.10 (s, | |
| | | 2H), 3.78-3.75 (m, | |
| | | 1H), 3.44 (s, 2H), | |
| | | 2.08-1.85 (m, 4H), | |
| | | 1.82-1.76 (m, 2H), | |
| | | 1.54-1.45 (m, 2H), | |
| | | 1.15-1.12 (m, 2H), | |
| | | 1.05-1.02 (m, 2H) | |
| 16 | 2-(6-bromo-1- | ¹H NMR (400 MHz, | m/z = |
| | oxospiro[3H- | DMSO-d₆): δ 8.25 | 405.3, |
| | isoquinoline-4,1′- | (d, J = 6.2 Hz, 1H), | 407.3 |
| | cyclopropane]-2-yl)- | 7.80 (d, J = 8.3 Hz, | [M + H]⁺ |
| | N-[(1R,2R,4S)-7- | 1H), 7.51 (dd, J = 8.3, | |
| | oxabicyclo[2.2.1]heptan- | 1.9 Hz, 1H), 7.24 | |
| | 2-yl]acetamide | (d, J = 1.9 Hz, 1H), | |
| | | 4.45 (t, J = 5.1 Hz, | |
| | | 2H), 4.18-4.08 (m, | |
| | | 2H), 3.90-3.85 (m, | |
| | | 1H), 3.45 (s, 2H), 2.01- | |
| | | 1.99 (m, 1H), 1.82 | |
| | | (ddd, J = 12.0, 9.0, 4.6 | |
| | | Hz, 1H), 1.60-1.40 | |
| | | (m, 3H), 1.23-1.19 (m, | |
| | | 1H), 1.15-1.13 (m, | |
| | | 2H), 1.05-1.02 (m, 2H) | |
| 17 | 2-(6-bromo-1- | ¹H NMR (400 MHz, | m/z = |
| | oxospiro[3H- | DMSO-d₆): δ 8.06- | 405.3, |
| | isoquinoline-4,1′- | 8.02 (m, 1H), 7.82-7.79 | 407.3 |
| | cyclopropane]-2-yl)- | (m, 1H), 7.53- | [M + H]⁺ |
| | N-[(1S,2R,4R)-7- | 7.50 (m, 1H), 7.24 | |
| | oxabicyclo[2.2.1]heptan- | (dd, J = 5.7, 1.3 Hz, | |
| | 2-yl]acetamide | 1H), 4.56-4.52 (m, | |
| | | 1H), 4.22-4.20 (m, | |
| | | 1H), 4.10-4.06 (m, | |
| | | 2H), 3.78-3.73 (m, | |
| | | 1H), 3.46-3.41 (m, | |
| | | 2H), 1.88-1.82 (m, | |
| | | 1H), 1.54-1.34 (m, | |
| | | 5H), 1.15-1.10 (m, | |
| | | 2H), 1.06-1.00 (m, 2H) | |
| 18 | 2-(6-bromo-1- | ¹H NMR (400 MHz, | m/z = |
| | oxospiro[3H- | CDCl₃): δ 8.24 (s, | 420.4, |
| | isoquinoline-4,1′- | 1H), 7.90 (d, J = 8.3 | 422.3 |
| | cyclopropane]-2-yl)- | Hz, 1H), 7.38 | [M + H]⁺ |
| | N-(1-ethylpiperidin- | (dd, J = 8.3, 1.8 Hz, | |
| | 3-yl)acetamide | 1H), 6.94 (d, J = 1.8 | |
| | | Hz, 1H), 4.42-4.37 | |
| | | (m, 1H), 4.23 (s, 2H), | |
| | | 3.46-3.39 (m, 2H), | |
| | | 3.31-3.30 (m, 1H), | |
| | | 3.06-2.96 (m, 2H), | |
| | | 2.74-2.74 (m, 1H), | |
| | | 2.22-2.19 (m, 2H), | |
| | | 1.90-1.87 (m, 1H), | |
| | | 1.85-1.83 (m, 1H), | |
| | | 1.81-1.79 (m, 1H), | |
| | | 1.73-1.68 (m, 1H), | |
| | | 1.31 (t, J = 7.2 Hz, | |
| | | 3H), 1.07-1.04 (m, 2H), | |
| | | 1.02-0.99 (m, 2H) | |
| 19 | 2-(6-bromo-1- | ¹H NMR (400 MHz, | m/z = |
| | oxospiro[3H- | DMSO-d₆): δ 8.19 | 393.5, |
| | isoquinoline-4,1′- | (d, J = 7.2 Hz, 1H), | 395.4 |
| | cyclopropane]-2-yl)- | 7.80 (d, J = 8.3 Hz, | [M + H]⁺ |
| | N-(cis-3-hydroxy-3- | 1H), 7.51 (dd, J = 8.3, | |
| | methylcyclo- | 1.9 Hz, 1H), 7.24 | |
| | butyl)acetamide | (d, J = 1.9 Hz, 1H), | |
| | | 4.07 (s, 2H), 3.81- | |
| | | 3.71 (m, 1H), 3.43 | |
| | | (s, 2H), 2.25-2.19 (m, | |
| | | 2H), 1.95-1.90 (m, | |
| | | 2H), 1.21 (s, 3H), | |
| | | 1.15-1.12 (m, 2H), | |
| | | 1.04-1.01 (m, 2H) | |
| 20 | 2-(6-bromo-1- | ¹H NMR (400 MHz, | m/z = |
| | oxospiro[3H- | DMSO-d₆): δ 8.24 | 429.3, |
| | isoquinoline-4,1′- | (d, J = 7.2 Hz, 1H), | 431.3 |
| | cyclopropane]-2-yl)- | 7.81 (d, J = 8.3 Hz, | [M + H]⁺ |
| | N-(4,5,6,7- | 1H), 7.52 (dt, J = 8.3, | |
| | tetrahydropyrazolo[1,5- | 1.4 Hz, 1H), 7.36 | |
| | alpyridin-5- | (d, J = 1.8 Hz, 1H), | |
| | yl)acetamide | 7.25 (t, J = 2.3 Hz, | |
| | | 1H), 6.01-6.01 (m, 1H), | |
| | | 4.23-4.05 (m, | |
| | | 5H), 3.46 (s, 2H), | |
| | | 3.08-3.03 (m, 1H), | |
| | | 2.69-2.63 (m, 1H), | |
| | | 2.14-2.08 (m, 1H), | |
| | | 2.08-1.98 (m, 1H), | |
| | | 1.15 (dt, J = 5.3, 2.8 | |
| | | Hz, 2H), 1.04 (dt, | |
| | | J = 5.2, 2.8 Hz, 2H) | |
| 21 | 2-(6-bromo-1- | ¹H NMR (400 MHz, | m/z = |
| | oxo-spiro[3H- | CDCl₃): δ 7.94 | 420.3, |
| | isoquinoline-4,1′- | (d, J = 8.3 Hz, 1H), | 422.3 |
| | cyclopropane]-2-yl)- | 7.46 (dd, J = 8.3, 1.8 | [M + H]⁺ |
| | N-(1-methyl-6-oxo-3- | Hz, 1H), 7.00 (d, J = | |
| | piperidyl)acetamide | 1.7 Hz, 1H), 6.73 | |
| | | (d, J = 7.3 Hz, 1H), | |
| | | 4.28-4.22 (m, 1H), | |
| | | 4.14 (q, J = 17.6 Hz, | |
| | | 2H), 3.55-3.51 (m, | |
| | | 1H), 3.51-3.44 (m, | |
| | | 2H), 3.13 (m, 1H), | |
| | | 2.90 (s, 3H), 2.47-2.44 | |
| | | (m, 2H), 2.03-1.96 | |
| | | (m, 1H), 1.88-1.80 (m, | |
| | | 1H), 1.13-1.09 (m, | |
| | | 2H), 1.03-1.01 (m, 2H) | |
| 22 | 2-(6-bromo-1- | ¹H NMR (400 MHz, | m/z = |
| | oxo-spiro[3H- | CDCl₃): δ 7.91 | 420.5, |
| | isoquinoline-4,1′- | (d, J = 8.3 Hz, 1H), | 422.4 |
| | cyclopropane]-2-yl)- | 7.43 (dd, J = 8.3, 1.8 | [M + H]⁺ |
| | N-(1-methyl-2-oxo-4- | Hz, 1H), 6.98 (d, J = | |
| | piperidyl)acetamide | 1.7 Hz, 1H), 6.96- | |
| | | 6.94 (m, 1H), 4.25-4.21 | |
| | | (m, 1H), 4.20- | |
| | | 4.17 (m, 1H), 4.11-4.06 | |
| | | (m, 1H), 3.53 | |
| | | (d, J = 12.5 Hz, 1H), | |
| | | 3.40 (d, J = 12.5 Hz, | |
| | | 1H), 3.32-3.28 (m, 2H), | |
| | | 2.87 (s, 3H), 2.67 | |
| | | (dd, J = 17.0, 5.2 Hz, | |
| | | 1H), 2.31-2.25 (m, | |
| | | 1H), 2.09-2.05 (m, | |
| | | 1H), 1.88-1.79 (m, | |
| | | 1H), 1.15-1.07 (m, 2H), | |
| | | 1.05-0.99 (m, 2H) | |
| 23 | 2-(6-bromo-1- | ¹H NMR (400 MHz, | m/z = |
| | oxo-spiro[3H- | CDCl₃): δ 8.01 (d, J = | 389.3, |
| | isoquinoline-4,1′- | 8.3 Hz, 1H), 7.50-7.48 | 391.3 |
| | cyclopropane]-2-yl)- | (m, 1H), 7.02 | [M + H]⁺ |
| | N-spiro[2.3]hexan- | (d, J = 1.6 Hz, 1H), | |
| | 5-yl-acetamide | 6.55-6.53 (m, 1H), | |
| | | 4.64-4.54 (m, 1H), | |
| | | 4.17 (s, 2H), 3.48 (s, | |
| | | 2H), 2.36 (ddd, J = 10.3, | |
| | | 7.9, 2.6 Hz, 2H), | |
| | | 2.17-2.12 (m, 2H), 1.15 | |
| | | (t, J = 5.9 Hz, | |
| | | 2H), 1.07-1.04 (m, 2H), | |
| | | 0.51-0.47 (m, | |
| | | 2H), 0.42-0.38 (m, 2H) | |
| 24 | 2-(6-bromo-1- | ¹H NMR (400 MHz, | m/z = |
| | oxo-spiro[3H- | CDCl₃): δ 7.94 | 433.3, |
| | isoquinoline-4,1′- | (dd, J = 8.3, 0.3 Hz, | 435.2 |
| | cyclopropane]-2-yl)- | 1H), 7.46 (dd, J = | [M + H]⁺ |
| | N-(2-cyclopropyl- | 8.3, 1.9 Hz, 1H), 7.00 | |
| | tetrahydropyran-4- | (d, J = 1.8 Hz, 1H), | |
| | yl)acetamide | 6.94-6.92 (m, 1H), | |
| | | 4.26-4.22 (m, 1H), | |
| | | 4.12 (d, J = 6.2 Hz, 2H), | |
| | | 3.86-3.83 (m, | |
| | | 1H), 3.55-3.49 (m, | |
| | | 2H), 3.49-3.45 (m, | |
| | | 1H), 2.70-2.65 (m, 1H), | |
| | | 1.90-1.84 (m, | |
| | | 1H), 1.74-1.71 (m, 2H), | |
| | | 1.56-1.52 (m, | |
| | | 1H), 1.13-1.10 (m, 2H), | |
| | | 1.05-1.01 (m, | |
| | | 2H), 0.91-0.85 (m, 1H), | |
| | | 0.54-0.49 (m, | |
| | | 1H), 0.47-0.39 (m, 1H), | |
| | | 0.30-0.24 (m, | |
| | | 1H), 0.05-0.01 (m, 1H) | |
| 25 | 2-(6-bromo-1- | ¹H NMR (400 MHz, | m/z = |
| | oxo-spiro[3H- | CDCl₃): δ 8.25 (s, | 450.4, |
| | isoquinoline-4,1′- | 1H), 8.00 (d, J = 8.3 | 452.4 |
| | cyclopropane]-2-yl)- | Hz, 1H), 7.46 (dd, J = | [M + H]⁺ |
| | N-[1-(2- | 8.3, 1.8 Hz, 1H), 7.01 | |
| | methoxyethyl)-3- | (d, J = 1.7 Hz, | |
| | piperidyl]acetamide | 1H), 4.27-4.16 (m, 3H), | |
| | | 3.51-3.47 (m, | |
| | | 3H), 3.30-3.28 (m, 3H), | |
| | | 2.77-2.63 (m, | |
| | | 4H), 2.48-2.44 (m, | |
| | | 5H), 1.82-1.80 (m, | |
| | | 1H), 1.66-1.63 (m, 1H), | |
| | | 1.15-1.06 (m, 4H) | |

### Example 26

### (R)-2-(6-bromo-4,4-dimethyl-1-oxo-3,4-dihydroisoquinolin-2(1H)-yl)-N-(1-cyclobutylpiperidin-3-yl)acetamide (26)

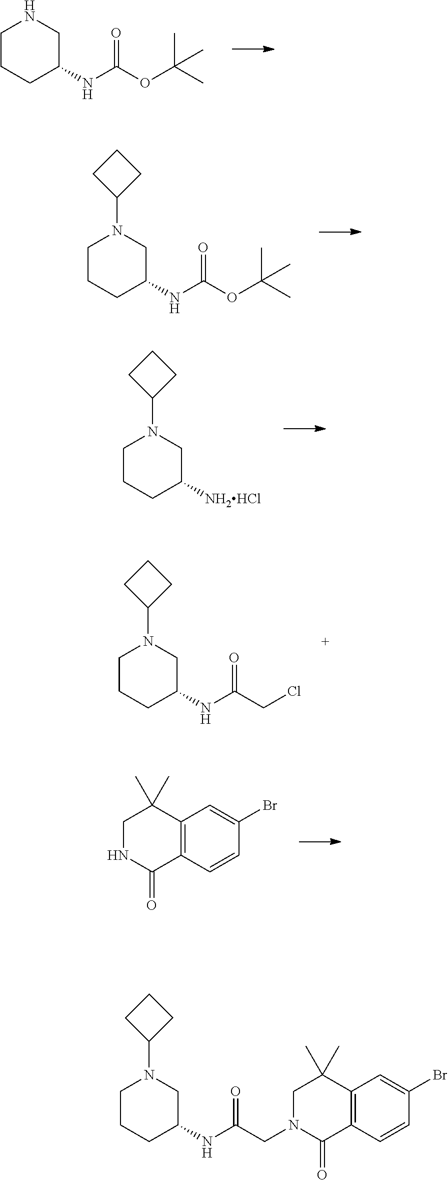

tert-Butyl N-[(3R)-1-cyclobutyl-3-piperidyl]carbamate: To a solution of tert-butyl N-[(3R)-3-piperidyl]carbamate (10.0 g, 49.9 mmol) and cyclobutanone (7.0 g, 99.9 mmol) in methanol (100 mL) was added sodium cyanoborohydride (5.33 g, 84.9 mmol) and acetic acid (5.71 mL, 99.9 mmol). The reaction mixture was stirred at 23° C. for 18 h. The reaction mixture was concentrated under reduced pressure. The residue was taken up in EtOAc (100 mL) and the organics were washed with water (2×50 mL) and brine (50 mL). The organic layer was dried over anhydrous MgSO₄, filtered, and concentrated under reduced pressure. The residue was purified by silica gel column chromatography. LCMS: m/z=255.2 [M+H]⁺.

(3R)-1-cyclobutylpiperidin-3-amine HCl salt: tert-Butyl N-[(3R)-1-cyclobutyl-3-piperidyl]carbamate (6.5 g, 25.6 mmol) was dissolved in HCl (63.9 mL, 4 N in 1,4-dioxane). The reaction mixture was stirred at 23° C. for 3 h. The reaction mixture was concentrated under reduced pressure to provide a residue that was used directly. LCMS: m/z=155.1 [M+H]⁺.

2-chloro-N-[(3R)-1-cyclobutyl-3-piperidyl]acetamide: To a solution of (3R)-1-cyclobutylpiperidin-3-amine HCl salt (5.7 g, 29.9 mmol) and N-methylmorpholine (13.2 mL, 119.6 mmol) in DMF (10 mL) and DCM (50 mL) was added a solution of 2-chloroacetyl chloride (2.38 mL, 29.9 mmol) in DCM (10 mL) at −78° C. The reaction mixture was stirred at 23° C. for 3 h. The reaction mixture was concentrated under reduced pressure. The residue was purified by silica gel column chromatography. LCMS: m/z=231.2 [M+H]⁺.

(R)-2-(6-bromo-4,4-dimethyl-1-oxo-3,4-dihydroisoquinolin-2(1H)-yl)-N-(1-cyclobutylpiperidin-3-yl)acetamide: To a solution of 2-chloro-N-[(3R)-1-cyclobutyl-3-piperidyl]acetamide (75 mg, 0.33 mmol) and 6-bromo-4,4-dimethyl-2,3-dihydroisoquinolin-1-one (64 mg, 0.25 mmol) in MeCN (1.0 mL) was added Cs₂CO₃ (205 mg, 0.63 mmol). The reaction mixture was stirred at 80° C. for 3 h. The reaction mixture was cooled to 23° C., poured into ice-cold water, and extracted with EtOAc (4×10 mL). The combined organic layers were dried over anhydrous MgSO₄, filtered, and concentrated under reduced pressure. The residue was purified by reverse-phase HPLC. LCMS: m/z=448.5, 450.5 [M+H]⁺. ¹H NMR (400 MHz, CDCl₃): δ 8.39 (s, 1H), 7.93-7.90 (m, 1H), 7.44-7.41 (m, 2H), 4.39-4.37 (m, 1H), 4.31-4.21 (m, 2H), 3.42 (q, J=10.4 Hz, 2H), 3.26-3.15 (m, 3H), 2.71-2.66 (m, 1H), 2.55-2.45 (m, 2H), 2.42-2.32 (m, 1H), 2.25-2.12 (m, 3H), 1.93-1.86 (m, 2H), 1.81-1.63 (m, 3H), 1.33 (d, J=2.5 Hz, 6H).

### Example 27

### 2-[1-oxo-6-(trifluoromethyl)spiro[3H-isoquinoline-4,1′-cyclopropane]-2-yl]-N-pyrimidin-2-yl-acetamide (27)

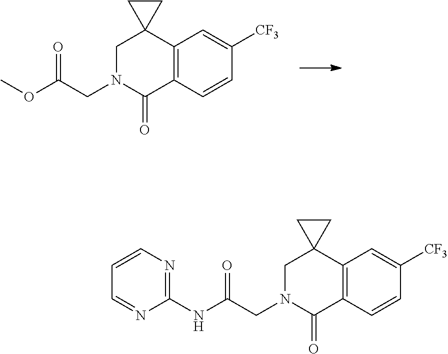 [SMILES: COC(=O)CN1CC2(CC2)c2cc(C(F)(F)F)ccc2C1=O ; O=C(CN1CC2(CC2)c2cc(C(F)(F)F)ccc2C1=O)Nc1ncccn1]

To a solution of pyrimidin-2-amine (91 mg, 0.96 mmol) and methyl 2-[1-oxo-6-(trifluoromethyl)spiro[3H-isoquinoline-4,1′-cyclopropane]-2-yl]acetate (100 mg, 0.32 mmol) in THF (3.0 mL) and toluene (1.0 mL) was added AlMe₃ (0.48 mL, 2 M in toluene). The reaction mixture was stirred at 90° C. for 4 h. The reaction mixture was diluted with water (2 mL) and extracted with EtOAc (2×5 mL). The combined organics were dried over anhydrous Na₂SO₄, filtered, and concentrated under reduced pressure. The residue was purified by reverse-phase HPLC. LCMS: m/z=377.0 [M+H]⁺. ¹H NMR (400 MHz, DMSO-d₆): δ 10.83 (s, 1H), 8.67 (d, J=5.2 Hz, 2H), 8.09 (d, J=8.0 Hz, 1H), 7.68 (d, J=8.0 Hz, 1H), 7.37 (s, 1H), 7.19 (t, J=5.2 Hz, 1H), 4.60 (s, 2H), 3.54 (s, 2H), 1.26-1.20 (m, 2H), 1.13-1.07 (m, 2H).

### Example 28

### 2-[1-oxo-6-(trifluoromethyl)spiro[3H-isoquinoline-4,1′-cyclopropane]-2-yl]-N-(5-fluoropyrimidin-2-yl-acetamide (28)

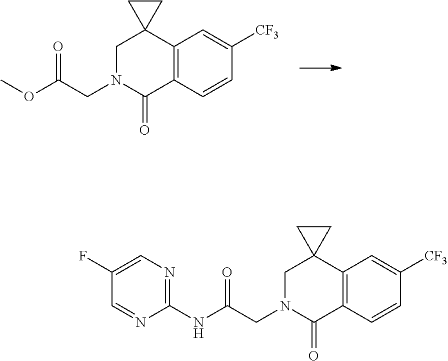 [SMILES: COC(=O)CN1CC2(CC2)c2cc(C(F)(F)F)ccc2C1=O ; O=C(CN1CC2(CC2)c2cc(C(F)(F)F)ccc2C1=O)Nc1ncc(F)cn1]

To a mixture of methyl 2-[1-oxo-6-(trifluoromethyl)spiro[3H-isoquinoline-4,1′-cyclopropane]-2-yl]acetate (50 mg, 0.16 mmol) and 5-fluoropyrimidin-2-amine (20 mg, 0.18 mmol) in toluene (1.0 mL) was added bis(trimethylaluminum)-1,4-diazabicyclo[2.2.2]-octane (41 mg, 0.16 mmol). The reaction mixture was stirred at 120° C. for 8 h. The reaction mixture was cooled to ambient temperature, diluted with water (2 mL) and EtOAc (2 mL), and filtered. The filtrate was extracted with EtOAc (3×2 mL). The combined organics were washed with brine (5 mL), dried over anhydrous Na₂SO₄, filtered, and concentrated under reduce pressure. The residue was purified by reverse-phase HPLC. LCMS: m/z=395.1 [M+H]⁺. ¹H NMR (400 MHz, DMSO-d₆) δ 10.99 (s, 1H), 8.77 (s, 2H), 8.10 (d, J=8.0 Hz, 1H), 7.69 (d, J=8.0 Hz. 1H), 7.38 (s, 1H), 4.54 (s, 2H), 3.54 (s, 2H), 1.25-1.22 (m, 2H), 1.12-1.09 (m, 2H).

The following compounds as shown in Table 1 were, or can be, made via similar procedures as those described above.

| Ex. | Name | NMR | LCMS |
|---|---|---|---|
| 29 | 2-[2′-methyl- | ¹H NMR (400 MHz, | m/z = |
| | 1-oxo-6- | DMSO-d₆) δ 10.84 (br s, 1H), | 391.0 |
| | (trifluoro- | 8.67 (d, J = 4.4 Hz, 2H), 8.07 | [M + H]⁺ |
| | methyl)spiro[3H- | (d, J = 8.0 Hz, 1H), 7.66 | |
| | isoquinoline-4,1′- | (d, J = 7.6 Hz, 1H), 7.32 | |
| | cyclopropane]- | (s, 1H), 7.19 (t, J = 4.8 Hz, | |
| | 2-yl]-N- | 1H), 4.70 (d, J = 17.2 Hz, | |
| | pyrimidin-2- | 1H), 4.50 (d, J = 16.8 Hz, | |
| | yl-acetamide | 1H), 3.81 (d, J = 8.4 Hz, 1H), | |
| | | 3.54 (d, J = 13.2 Hz, | |
| | | 1H), 1.52-1.49 (m, 1H), | |
| | | 1.35-1.34 (m, 1H), 1.26-1.25 | |
| | | (d, J = 6.4 Hz, 3H), 0.80-0.76 | |
| | | (m, 1H) | |
| 30 | 2-[4-ethyl-4-methyl-1- | ¹H NMR (400 MHz, CDCl₃): | m/z = |
| | oxo-6-(trifluoromethyl)- | δ 8.78 (br s, 1H), 8.62 (d, | 393.1 |
| | 3H-isoquinolin-2-yl]-N- | J = 4.8 Hz, 2H), 8.27 (d, J = | [M + H]⁺ |
| | pyrimidin-2-ylacetamide | 8.0 Hz, 1H), 7.63 (d, J = | |
| | | 8.0 Hz, 1H), 7.51 (s, 1H), | |
| | | 7.04 (t, J = 4.8 Hz, 1H), | |
| | | 4.89-4.62 (m, 2H), 3.72-3.47 | |
| | | (m, 2H), 1.85-1.72 (m, | |
| | | 2H), 1.37 (s, 3H), 0.85 | |
| | | (t, J = 8.4 Hz, 3H) | |
| 31 | 2-[5-oxo-2- | ¹H NMR (400 MHz, CDCl₃) δ | m/z = |
| | (trifluoro- | 8.61 (m, 2H), 8.53 (m, | 378.0 |
| | methyl)spiro[7H- | 2H), 7.60 (d, J = 8.0 Hz, 1H), | [M + H]⁺ |
| | 1,6-naphthyridine-8,1′- | 7.05 (t, J = 4.8 Hz, 1H), | |
| | cyclopropane]-6-yl]-N- | 4.82 (s, 2H), 3.71 (s, 2H), | |
| | pyrimidin-2-ylacetamide | 1.63-1.60 (m, 2H), 1.13-1.08 | |
| | | (m, 2H) | |

### Example 32

### 2-(6-bromo-1-oxo-spiro[3H-isoquinoline-4,1′-cyclobutane]-2-yl)-N-pyrimidin-2-yl-acetamide (32)

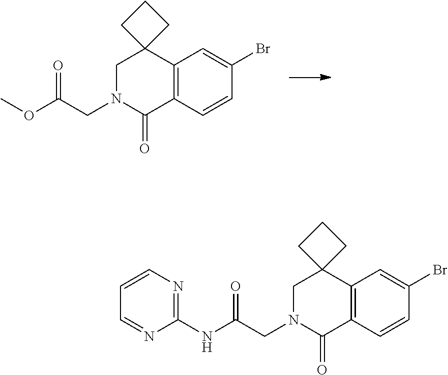 [SMILES: COC(=O)CN1CC2(CCC2)c2cc(Br)ccc2C1=O ; O=C(CN1CC2(CCC2)c2cc(Br)ccc2C1=O)Nc1ncccn1]

To a solution of methyl 2-(6-bromo-1-oxospiro[3H-isoquinoline-4,1′-cyclobutane]-2-yl)acetate and pyrimidin-2-amine (23 mg, 0.23 mmol) in toluene (0.8 mL) and THF (0.8 mL) was added AlMe₃ (0.24 mL, 2 M in toluene,). The reaction mixture was stirred at 50° C. for 12 h before another portion of AlMe₃ (0.24 mL, 2 M in toluene) was added. The reaction mixture was stirred at 90° C. for a further 4 h. The reaction mixture was cooled to ambient temperature, diluted with water (20 mL), and extracted with EtOAc (3×20 mL). The combined organic layers were washed with brine (20 mL), dried over anhydrous Na₂SO₄, filtered, and concentrated under reduced pressure. The residue was purified by reverse-phase HPLC. LCMS: m/z=401.1, 403.1 [M+H]⁺. ¹H NMR (400 MHz, CDCl₃): δ 8.84 (br s, 1H), 8.62 (d, J=4.8 Hz, 2H), 7.99 (d, J=8.4 Hz, 1H), 7.66 (d, J=1.6 Hz, 1H) 7.51-7.49 (in, 1H), 7.03 (t, J=4.8 Hz, 1H), 4.74 (s, 2H), 3.78 (s, 2H), 2.36-2.31 (m, 2H), 2.24-2.20 (m, 2H), 2.09-2.07 (m, 2H).

The following compounds as shown in Table 1 were, or can be, made via similar procedures as those described above.

| Ex. | Name | NMR | LCMS |
|---|---|---|---|
| 33 | N-(pyrimidin- | ¹H NMR (400 MHz, | m/z = |
| | 2-yl)-2-[6- | CDCl₃): δ 8.99 (br s, | 375.1 |
| | (difluoromethoxy)-1- | 1H), 8.62 (d, J = 4.8 | [M + H]⁺ |
| | oxospiro[3H- | Hz, 2H), 8.18 (d, J = 8.6 | |
| | isoquinoline-4,1′- | Hz, 1H), 7.12-6.98 | |
| | cyclopropane]- | (m, 2H), 6.63 (s, 1H), | |
| | 2-yl]acetamide | 6.57(t, J = 72.0 Hz, 1H), | |
| | | 4.73 (s, 2H), 3.59 (s, | |
| | | 2H), 1.17-1.07 (m, 4H) | |
| 34 | N-(5-chloropyrimidin- | ¹H NMR (400 MHz, | m/z = |
| | 2-yl)-2-(6- | CDCl₃): δ 9.21 (br s, | 383.1 |
| | cyclopropyl-1- | 1H), 8.55 (br s, 2H), 8.05 | [M + H]⁺ |
| | oxo-spiro[3H- | (d, J = 8.4 Hz, 1H), | |
| | isoquinoline-4,1′- | 6.96 (dd, J = 1.6, 8.0 | |
| | cyclopropane]- | Hz, 1H), 6.57 (d, J = | |
| | 2-yl)acetamide | 9.6 Hz, 1H), 4.53 (s, 2H), | |
| | | 3.51 (s, 2H), 1.95- | |
| | | 1.86 (m, 1H), 1.18-1.08 | |
| | | (m, 2H), 1.08-0.99 | |
| | | (m, 4H), 0.77-0.72 (m, 2H) | |
| 35 | 2-[5-fluoro- | ¹H NMR (400 MHz, | m/z = |
| | 1-oxo-6- | DMSO-d₆): δ 11.00 (br | 413.0 |
| | (trifluoro- | s, 1H), 8.77 (s, 2H), | [M + H]⁺ |
| | methyl)spiro[3H- | 7.98-7.90 (m, 1H), 7.76- | |
| | isoquinoline-4,1′- | 7.70 (m, 1H), 4.53 (s, | |
| | cyclopropane]- | 2H), 3.49 (s, 2H), 1.55- | |
| | 2-yl]-N-(5- | 1.46 (m, 2H), 1.16-1.10 | |
| | fluoropyrimidin-2- | (m, 2H) | |
| | yl)acetamide | | |
| 36 | 2-(6-bromo-1- | ¹H NMR (400 MHz, | m/z = |
| | oxo-spiro[3H- | DMSO-d₆): δ 10.83 (s, | 452.3, |
| | isoquinoline-4,1′- | 1H), 8.26 (dd, J = 3.0, | 454.3 |
| | cyclopropane]- | 0.5 Hz, 1H), 8.11-8.07 | [M + H]⁺ |
| | 2-yl)-N-[5- | (m, 1H), 7.81 (d, | |
| | (difluoromethoxy)-2- | J = 8.3 Hz, 1H), 7.70 | |
| | pyridyl]acetamide | (dd, J = 9.0, 2.9 Hz, | |
| | | 1H), 7.53 (dd, J = 8.3, | |
| | | 1.9 Hz, 1H), 7.27 (d, | |
| | | J = 1.9 Hz, 1H), 7.23 | |
| | | (t, J = 73.6 Hz, 1H), | |
| | | 4.39 (s, 2H), 3.52 (s, | |
| | | 2H), 1.18-1.15 (t, J = 3.0 | |
| | | Hz, 2H), 1.07-1.05 | |
| | | (m, 2H) | |
| 37 | 2-(6-bromo-1- | ¹H NMR (400 MHz, | m/z = |
| | oxo-spiro[3H- | DMSO-d₆): δ 10.95 (br | 432.3, |
| | isoquinoline-4,1′- | s, 1H), 10.05 (d, J = 2.1 | 434.3 |
| | cyclopropane]- | Hz, 1H), 8.79 | [M + H]⁺ |
| | 2-yl)-N-(3-fluoro- | (dd, J = 1.8, 0.8 Hz, | |
| | 5-formyl-2- | 1H), 8.13 (dd, J = 10.2, | |
| | pyridyl)acetamide | 1.8 Hz, 1H), 7.82 (d, | |
| | | J = 8.3 Hz, 1H), 7.53 | |
| | | (dd, J = 8.3, 1.9 Hz, 1H), | |
| | | 7.27 (d, J = 1.9 Hz, | |
| | | 1H), 4.51 (s, 2H), 3.52 | |
| | | (s, 2H), 1.17-1.15 (m, | |
| | | 2H), 1.08-1.05 (m, 2H) | |
| 38 | 2-(6-bromo-1- | ¹H NMR (400 MHz, | m/z = |
| | oxo-spiro[3H- | DMSO-d₆): δ 10.54 (br | 422.4, |
| | isoquinoline-4,1′- | s, 1H), 8.36-8.30 (m, 1H), | 424.4 |
| | cyclopropane]- | 8.08-8.00 (m, 1H), | [M + H]⁺ |
| | 2-yl)-N-(3,5- | 7.82 (d, J = 8.3 Hz, | |
| | difluoro-2- | 1H), 7.53 (d, J = 8.3, | |
| | pyridyl)acetamide | 1H), 7.26 (s, 1H), 4.41 | |
| | | (s, 2H), 3.51 (s, 2H), | |
| | | 1.17-1.14 (m, 2H), 1.07-1.04 | |
| | | (m, 2H). | |

### Examples 39 and 40

### 2-(6-bromo-5-fluoro-1-oxo-spiro[3H-isoquinoline-4,1′-cyclopropane]-2-yl)-N-(pyrimidin-2-yl)acetamide (39) and 2-(5-fluoro-1-oxo-spiro[3H-isoquinoline-4,1′-cyclopropane]-2-yl)-N-(pyrimidin-2-yl)acetamide (40)

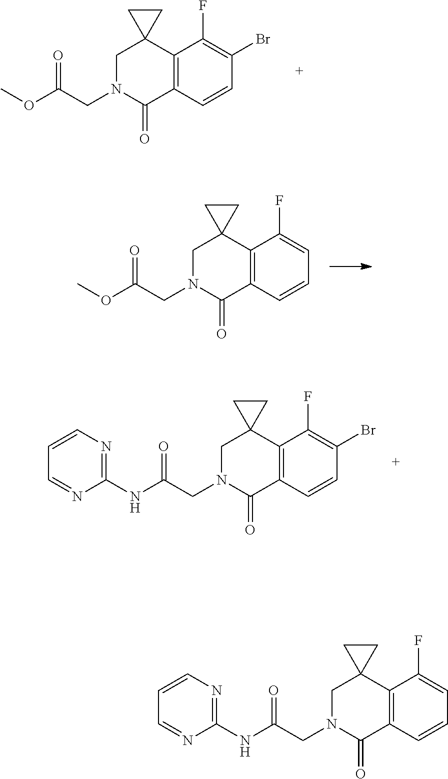 [SMILES: COC(=O)CN1CC2(CC2)c2c(ccc(Br)c2F)C1=O ; COC(=O)CN1CC2(CC2)c2c(F)cccc2C1=O ; O=C(CN1CC2(CC2)c2c(ccc(Br)c2F)C1=O)Nc1ncccn1 ; O=C(CN1CC2(CC2)c2c(F)cccc2C1=O)Nc1ncccn1]

To a mixture of methyl 2-(6-bromo-5-fluoro-1-oxo-spiro[3H-isoquinoline-4,1′-cyclopropane]-2-yl)acetate and methyl 2-(5-fluoro-1-oxo-spiro[3H-isoquinoline-4,1′-cyclopropane]-2-yl)acetate (230 mg, 0.67 mmol, 1:3 ratio) and pyrimidin-2-amine (192 mg, 2.02 mmol) in toluene (3.0 mL) and THF (2.0 mL) was added and AlMe₃ (1.01 mL, 2 M in toluene). The reaction mixture was stirred at 100° C. for 5 h. The reaction mixture was cooled to ambient temperature, diluted with ice cold water (5 mL) and extracted with EtOAc (3×5 mL). The combined organics were washed with brine (20 mL), dried over anhydrous Na₂SO₄, filtered, and concentrated under reduced pressure. The residue was purified by reverse-phase HPLC to provide:

2-(6-bromo-5-fluoro-1-oxo-spiro[3H-isoquinoline-4,1′-cyclopropane]-2-yl)-N-(pyrimidin-2-yl)acetamide (39). LCMS: m/z=404.9, 406.9 [M+H]⁺. ¹H NMR (400 MHz, CDCl₃): δ 9.26-9.11 (m, 1H), 8.63 (d, J=4.8 Hz, 2H), 7.87 (d, J=8.4 Hz, 1H), 7.51-7.45 (m, 1H), 7.03 (t, J=4.8 Hz, 1H), 4.81-4.66 (m, 2H), 3.51-3.41 (m, 2H), 1.65-1.61 (m, 2H), 1.08-1.00 (m, 2H).

2-(5-fluoro-1-oxo-spiro[3H-isoquinoline-4,1′-cyclopropane]-2-yl)-N-(pyrimidin-2-yl)acetamide (40). LCMS: m/z=327.0 [M+H]⁺. ¹H NMR (400 MHz, CDCl₃): δ 9.32-9.22 (m, 1H), 8.63 (d, J=4.8 Hz, 2H), 8.03-7.97 (m. 1H), 7.26-7.22 (m, 1H), 7.14-7.06 (m, 1H), 7.03 (t, J=4.8 Hz, 1H), 4.72 (br s, 2H), 3.46 (s, 2H), 1.65-1.59 (m, 2H), 1.04-0.98 (m, 2H).

### Examples 41 and 42

### 2-(6-bromo-5-fluoro-1-oxo-spiro[3H-isoquinoline-4,1′-cyclopropane]-2-yl)-N-(5-fluoropyrimidin-2-yl)acetamide (41) and 2-(5-fluoro-1-oxo-spiro[3H-isoquinoline-4,1′-cyclopropane]-2-yl)-N-(5-fluoropyrimidin-2-yl)acetamide (42)

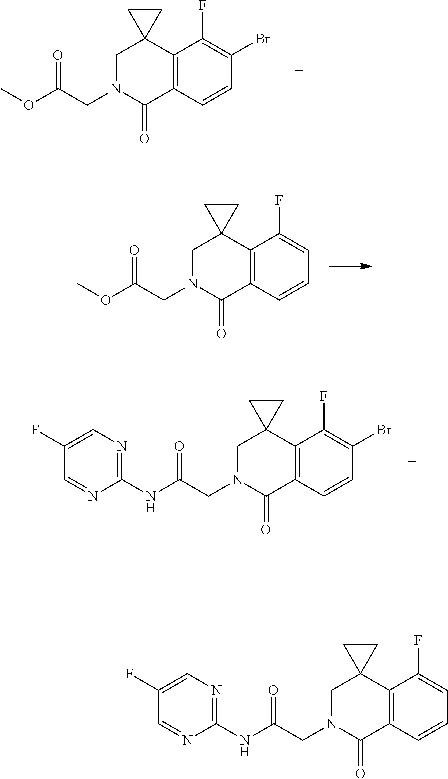 [SMILES: COC(=O)CN1CC2(CC2)c2c(ccc(Br)c2F)C1=O ; COC(=O)CN1CC2(CC2)c2c(F)cccc2C1=O ; O=C(CN1CC2(CC2)c2c(ccc(Br)c2F)C1=O)Nc1ncc(F)cn1 ; O=C(CN1CC2(CC2)c2c(F)cccc2C1=O)Nc1ncc(F)cn1]

To a mixture methyl 2-(6-bromo-5-fluoro-1-oxo-spiro[3H-isoquinoline-4,1′-cyclopropane]-2-yl)acetate and methyl 2-(5-fluoro-1-oxo-spiro[3H-isoquinoline-4,1-cyclopropane]-2-yl)acetate (400 mg, 1.17 mmol, 1:3 ratio) in toluene (3.0 mL) and THF (2.0 mL) were added 5-fluoropyrimidin-2-amine (397 mg, 3.51 mmol) and AlMe₃ (1.75 mL, 2 M in toluene). The reaction mixture was stirred at 100° C. for 3 h. The reaction mixture was cooled to ambient temperature, poured into ice cold water, and extracted with EtOAc (3×8 mL). The combined organics were washed with brine (20 mL), dried over anhydrous Na₂SO₄, filtered, and concentrated under reduced pressure. The residue was purified by reverse-phase HPLC to provide:

2-(6-bromo-5-fluoro-1-oxo-spiro[3H-isoquinoline-4,1′-cyclopropane]-2-yl)-N-(5-fluoropyrimidin-2-yl)acetamide (41): LCMS: m/z=423.1, 425.1 [M+H]⁺. ¹H NMR (400 MHz, DMSO-d₆): δ 10.98 (br s, 1H). 8.76 (s, 2H), 7.72-7.64 (m, 2H), 4.50 (s, 2H), 3.45 (s, 2H), 1.51-1.44 (m, 2H), 1.11-1.05 (m, 2H).

2-(5-fluoro-1-oxo-spiro[3H-isoquinoline-4,1′-cyclopropane]-2-yl)-N-(5-fluoropyrimidin-2-yl)acetamide (42): LCMS: m/z=345.1 [M+H]⁺. ¹H NMR (400 MHz, DMSO-d₆): δ 10.90-11.05 (br s, 1H), 8.76 (s, 2H), 7.76-7.81 (m, 1H), 7.27-7.38 (m, 2H), 4.41-4.60 (s, 2H), 3.44 (s. 2H), 1.40-1.46 (m, 2H), 1.05 (m, 2H).

### Example 43

### 2-(6-bromo-1-oxo-spiro[3H-isoquinoline-4,1′-cyclopropane]-2-yl)-N-(1H-indazol-6-yl)acetamide (43)

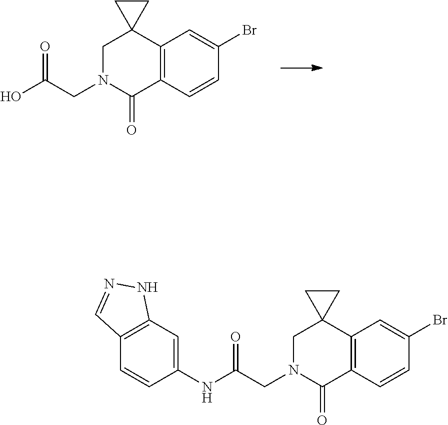 [SMILES: O=C(O)CN1CC2(CC2)c2cc(Br)ccc2C1=O ; O=C(CN1CC2(CC2)c2cc(Br)ccc2C1=O)Nc1ccc2cn[nH]c2c1]

To a solution of 1H-indazol-6-amine (70.84 mg, 0.53 mmol) and 2-(6-bromo-1-oxo-spiro[3H-isoquinoline-4,1′-cyclopropane]-2-yl)acetic acid (150 mg, 0.48 mmol) in DMF (5.0 mL) were added HATU (276 mg, 0.73 mmol) and DIPEA (94 mg, 0.73 mmol). The reaction mixture was stirred at 25° C. for 4 h. The reaction mixture was diluted with water (30 mL) and extracted with EtOAc (3×20 mL). The combined organic layers were washed with brine (20 mL), dried over anhydrous Na₂SO₄, filtered, and concentrated under reduced pressure. The residue was purified by reverse-phase HPLC. LCMS: m/z=425.0, 427.0 [M+H]⁺. ¹H NMR (400 MHz, DMSO-d₆) δ 12.89 (br s, 1H), 10.27 (br s, 1H), 8.11 (s, 1H), 7.96 (s, 1H), 7.82 (d, J=8.4 Hz, 1H), 7.66 (d, J=8.8 Hz, 1H), 7.55-7.51 (m, 1H), 7.27 (d, J=2.0 Hz, 1H), 7.11-7.07 (m. 1H), 4.36 (s, 2H), 3.54 (s, 2H), 1.20-1.14 (m, 2H), 1.10-1.03 (m, 2H).

The following compounds as shown in Table 1 were, or can be, made via similar procedures as those described above.

| Ex. | Name | NMR | LCMS |
|---|---|---|---|
| 44 | 2-(6-bromo-1- | ¹H NMR (400 MHz, DMSO-d₆): | m/z = |
| | oxospiro[3H- | δ 10.99 (s, 1H), 10.01 (s, 1H), | 424.0, |
| | isoquinoline-4,1′- | 7.94 (s, 1H), 7.83 (d, J = 8.4 Hz, | 426.0 |
| | cyclopropane]- | 1H), 7.53 (dd, J = 8.4, 1.6 Hz, | [M + H]⁺ |
| | 2-yl)-N-(1H-indol-6- | 1H), 7.43 (d, J = 8.4 Hz, 1H), | |
| | yl)acetamide | 7.27-7.24 (m, 2H), 7.03 (dd, J = | |
| | | 8.4, 1.6 Hz, 1H), 6.34 (d, J = 2.0 | |
| | | Hz, 1H), 4.33 (s, 2H), 3.54 (s, | |
| | | 2H), 1.20-1.14 (m, 2H), 1.11- | |
| | | 1.04 (m, 2H) | |
| 45 | 2-(6-bromo-1- | ¹H NMR (400 MHz, CDCl₃): δ | m/z = |
| | oxospiro[3H- | 8.89 (d, J = 8.4 Hz, 1H), 7.42 (d, | 429.1, |
| | isoquinoline-4,1′- | J = 9.6 Hz, 1H), 7.28 (d, J = 9.6 | 431.0 |
| | cyclopropane]-2- | Hz, 1H), 6.98 (s, 1H), 6.88 (d, J = | [M + H]⁺ |
| | yl)-N-(4,5,6,7- | 7.6 Hz, 1H), 6.61 (br s, 1H), | |
| | tetrahydro-1H- | 4.30 (s, 1H), 4.25-4.11 (m, 2H), | |
| | indazol-6-yl)acetamide | 3.52-3.45 (m, 2H), 3.00-2.95 (m, | |
| | | 1H), 2.61-2.52 (m, 3H), 1.9-1.84 | |
| | | (m, 1H), 1.82-1.80 (m, 1H), | |
| | | 1.10-0.99 (m, 4H) | |
| 46 | 2-(6-bromo-4,4- | ¹H NMR (400 MHz, CDCl₃): δ | m/z = |
| | dimethyl-1-oxo-3H- | 7.91 (d, J = 8.2 Hz, 1H), 7.50- | 395.3, |
| | isoquinolin-2-yl)- | 7.46 (m, 2H), 7.01 (d, J = 7.6 | 397.3 |
| | N-(cis-3-hydroxy- | Hz, 1H), 4.18 (s, 2H), 3.98 | [M + H]⁺ |
| | 3-methyl- | (sextet, J = 7.9 Hz, 1H), 3.48 (s, | |
| | cyclobutyl)acetamide | 2H), 3.12 (br s, 1H), 2.49 (ddd, J = | |
| | | 10.0, 7.6, 2.6 Hz, 2H), 2.08- | |
| | | 2.02 (m, 2H), 1.35 (d, J = 6.0 | |
| | | Hz, 9H) | |
| 47 | 2-(6-bromo-1- | ¹H NMR (400 MHz, CDCl₃): δ | m/z = |
| | oxospiro[3H- | 7.96 (d, J = 8.4 Hz, 1H), 7.45 | 395.1, |
| | isoquinoline-4,1′- | (dd, J = 1.6, 8.4 Hz, 1H), 6.99 (d, | 397.1 |
| | cyclopropane]-2- | J = 1.6 Hz, 2H), 4.15 (s, 2H), | [M + H]⁺ |
| | yl)-N-(3-hydroxy-3- | 3.46 (s, 2H), 3.46-3.40 (m, 2H), | |
| | methylbutyl)acetamide | 1.70-1.66 (m, 2H), 1.24 (s, 6H), | |
| | | 1.14-1.09 (m, 2H), 1.08-1.02 (m, | |
| | | 2H) | |

### Example 48

### 2-(6-bromo-1-oxospiro[3H-isoquinoline-4,1′-cyclopropane]-2-yl)-N-(5-pyrazol-1-ylpyrimidin-2-yl)acetamide (48)

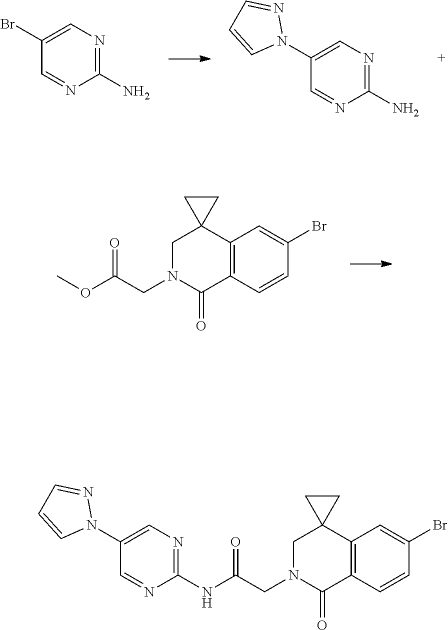 [SMILES: Nc1ncc(Br)cn1 ; Nc1ncc(-n2cccn2)cn1 ; COC(=O)CN1CC2(CC2)c2cc(Br)ccc2C1=O ; O=C(CN1CC2(CC2)c2cc(Br)ccc2C1=O)Nc1ncc(-n2cccn2)cn1]

5-(1H-pyrazol-1-yl) pyrimidin-2-amine: To a solution of 5-bromopyrimidin-2-amine (0.30 g, 1.72 mmol) and 1H-pyrazole (98 mg, 1.44 mmol) in DMF (3.0 mL) were added (1S,2S)—N₁,N₂-dimethylcyclohexane-1,2-diamine (41 mg, 0.28 mmol), K₂CO₃ (298 mg, 2.16 mmol) and CuI (27 mg. 0.14 mmol). The reaction mixture was stirred at 120° C. for 2 h. The reaction mixture was cooled to 0° C., diluted with water (10 mL), and extracted with EtOAc (4×10 mL). The combined organics were washed with brine (10 mL), dried over anhydrous Na₂SO₄, filtered, and concentrated under reduced pressure. The residue was purified by preparatory TLC. LCMS: m/z=162.0 [M+H]⁺.

2-(6-bromo-1-oxospiro[3H-isoquinoline-4,1′-cyclopropane]-2-yl)-N-(5-pyrazol-1-ylpyrimidin-2-yl)acetamide: To a solution of 5-(1H-pyrazol-1-yl) pyrimidin-2-amine (30 mg, 0.19 mmol) and 2-(6-bromo-1-oxo-spiro[3H-isoquinoline-4,1′-cyclopropane]-2-yl)acetate (60 mg, 0.19 mmol) in toluene (1.5 mL) was added bis(trimethylaluminum)-1,4-diazabicyclo[2.2.2]-octane (48 mg, 0.19 mmol). The reaction mixture was stirred at 100° C. for 2 h. The reaction mixture was cooled to 0° C., diluted with water (10 mL), and extracted with EtOAc (4×10 mL). The combined organics were washed with brine (10 mL), dried over anhydrous Na₂SO₄, filtered, and concentrated under reduced pressure. The crude residue was purified by reverse-phase HPLC. LCMS: m/z=453.0, 455.0 [M+H]⁺. ¹H NMR (400 MHz, CDCl₃) δ 9.12 (s, 1H), 8.71 (s, 2H), 8.03 (d, J=8.0 Hz, 1H), 7.80 (s, 1H), 7.49-7.46 (m, 1H), 7.31 (s, 1H), 7.24 (s, 1H), 7.02 (s, 1H), 4.62 (s, 2H), 3.56 (s, 2H), 1.15-1.11 (m, 2H), 1.10-1.07 (m, 2H).

### Example 49

### 2-(6-bromo-1-oxo-spiro[3H-isoquinoline-4,1′-cyclopropane]-2-yl)-N-[3-(1-hydroxy-1-methyl-ethyl)-1-bicyclo[1.1.1]pentanyl]acetamide (49)

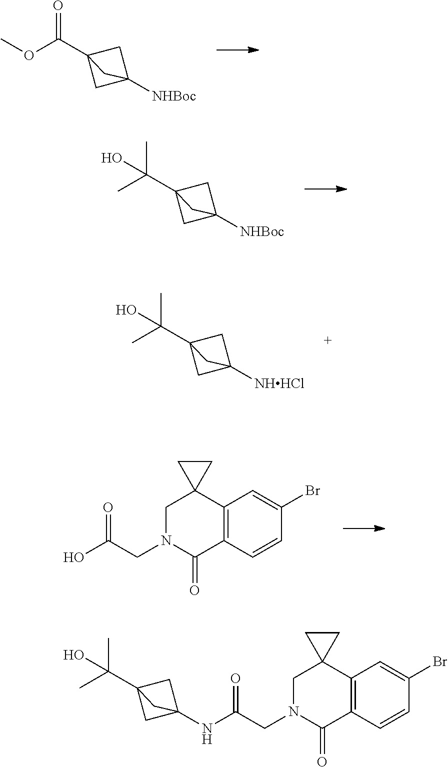

tert-butyl (3-(2-hydroxypropan-2-yl)bicyclo[1.1.1]pentan-1-yl)carbamate: To a solution of methyl 3-((tert-butoxycarbonyl)amino)bicyclo[1.1.1]pentane-1-carboxylate (2.0 g, 8.29 mmol) in THF (20 mL) at 0° C. was added MeMgBr (11.1 mL, 3 M in diethyl ether). The reaction mixture was stirred at 20° C. for 2 h. The reaction mixture was diluted with sat. aq. NH₄Cl (20 mL) and extracted with EtOAc (3×8 mL). The combined organics were washed with brine (20 mL), dried over anhydrous Na₂SO₄, filtered, and concentrated under reduced pressure to provide a residue that was used directly. ¹H NMR (400 MHz, DMSO-d₆): δ 7.35 (br s, 1H), 4.08 (s, 1H), 1.69 (s, 6H), 1.36 (s, 9H), 1.02 (s, 6H).

2-(3-aminobicyclo[1.1.1]pentan-1-yl)propan-2-ol HCl salt: A solution of tert-butyl (3-(2-hydroxypropan-2-yl)bicyclo[1.1.1]pentan-1-yl)carbamate (1.0 g, 4.14 mmol) in HCl (10 mL, 4 N in MeOH) was stirred at 20° C. for 2 h. The reaction mixture was concentrated under reduced pressure to provide a residue that was used directly. ¹H NMR (400 MHz, DMSO-d₆): δ 8.89 (br s, 3H), 1.86-1.72 (m, 6H), 1.07-1.00 (m, 6H).

2-(6-bromo-1-oxo-spiro[3H-isoquinoline-4,1′-cyclopropane]-2-yl)-N-[3-(1-hydroxy-1-methyl-ethyl)-1-bicyclo[1.1.1]pentanyl]acetamide: To a solution of 2-(3-aminobicyclo[1.1.1]pentan-1-yl)propan-2-ol HCl salt (55 mg, 0.31 mmol) in DMF (1.0 mL) were added 2-(6-bromo-1-oxo-spiro[3H-isoquinoline-4,1′-cyclopropane]-2-yl)acetic acid (80 mg, 0.26 mmol), DIPEA (134 mg, 1.03 mmol), and HATU (196 mg, 0.52 mmol). The reaction mixture was stirred at 20° C. for 2 h. The reaction mixture was diluted with water (5 mL) and extracted with EtOAc (3×2 mL). The combined organic layers were washed with brine (5 mL), dried over anhydrous Na₂SO₄, filtered, and concentrated under reduced pressure. The residue was purified by reverse-phase HPLC. LCMS: m/z=433.1, 435.1 [M+H]⁺. ¹H NMR (400 MHz, CDCl₃): δ 7.97 (d, J=8.4 Hz, 1H), 7.47 (d, 8.4 Hz, 1H), 7.00 (s, 1H), 6.64 (s, 1H), 4.13 (s, 2H), 3.46 (s, 2H), 1.98 (s, 6H), 1.19 (s, 6H), 1.15-1.09 (m, 2H), 1.05-1.00 (m, 2H).

### Example 50

### 2-(6-bromo-1-oxospiro[3H-isoquinoline-4,1′-cyclopropane]-2-yl)-N-[3-(2-hydroxypropan-2-yl)cyclobutyl]acetamide (50)

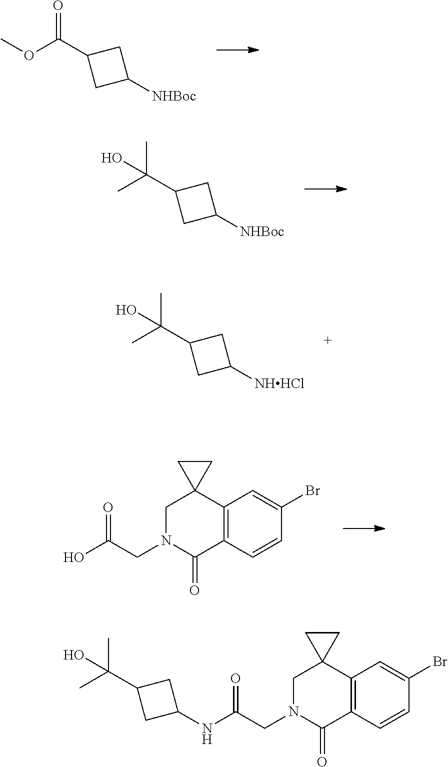

tert-butyl (3-(2-hydroxypropan-2-yl)cyclobutyl)carbamate: To a solution of methyl 3-((tert-butoxycarbonyl)amino)cyclobutanecarboxylate (500 mg, 2.18 mmol) in THF (10 mL) at −78° C. was added MeMgBr (2.91 mL, 3 M in Et₂O). The reaction mixture was stirred at 25° C. for 1 h. The reaction mixture was diluted with water (20 mL) and extracted with EtOAc (3×20 mL). The combined organics were washed with brine (20 mL), dried over anhydrous Na₂SO₄, filtered, and concentrated under reduced pressure to provide a residue that was used directly. LCMS: m/z=230.2 [M+H]⁺.

2-(3-aminocyclobutyl)propan-2-ol HCl salt: A solution of tert-butyl(3-(2-hydroxypropan-2-yl)cyclobutyl) carbamate (400 mg, 1.74 mmol) in HCl (4.0 mL, 4 N in MeOH) was stirred at 25° C. for 1 h. The reaction mixture was concentrated under reduced pressure to provide a residue that was used directly. ¹H NMR (400 MHz, CDCl₃): δ 5.41 (br s, 3H), 2.45-2.25 (m, 3H), 2.22-2.10 (m, 1H), 1.97 (d, J=8.8 Hz, 1H), 1.55-1.41 (m. 1H), 1.25-1.03 (m, 6H).

2-(6-bromo-1-oxospiro[3H-isoquinoline-4,1′-cyclopropane]-2-yl)-N-[3-(2-hydroxypropan-2-yl)cyclobutyl]acetamide: To a solution of 2-(6-bromo-1-oxo-spiro[3H-isoquinoline-4,1′-cyclopropane]-2-yl)acetic acid (50 mg, 0.16 mmol), HATU (96 mg, 0.25 mmol) and DIPEA (109 mg, 0.84 mmol) in DMF (2.0 mL) was added 2-(3-aminocyclobutyl)propan-2-ol HCl salt (28 mg, 0.17 mmol). The reaction mixture was stirred at 20° C. for 2 h. The reaction mixture was diluted with water (5 mL) and extracted with EtOAc (3×5 mL). The combined organic layers were washed with brine (5 mL), dried over anhydrous Na₂SO₄, filtered, and concentrated under reduced pressure. The residue was purified by reverse-phase HPLC. LCMS: m/z=421.1, 423.0 [M+H]⁺. ¹H NMR (400 MHz, CDCl₃): δ 7.97 (dd, J=8.4, 4.0 Hz. 1H), 7.48-7.45 (m, 1H), 7.02-7.00 (m, 1H), 6.84-6.66 (m, 1H), 4.37-4.23 (m, 1H), 4.18 (d, J=5.6 Hz, 2H), 3.48 (d, J=8.4 Hz, 2H), 2.41-2.30 (m, 4H), 2.04-1.94 (m, 1H), 1.88-1.79 (m, 1H), 1.16 (s, 3H), 1.15-1.12 (m, 2H), 1.11 (s, 3H), 1.09-1.02 (m, 2H).

### Example 51

### 2-(6-bromo-1-oxo-spiro[3H-isoquinoline-4,1′-cyclopropane]-2-yl)-N-(2-oxabicyclo[2.2.2]octan-4-yl)acetamide (51)

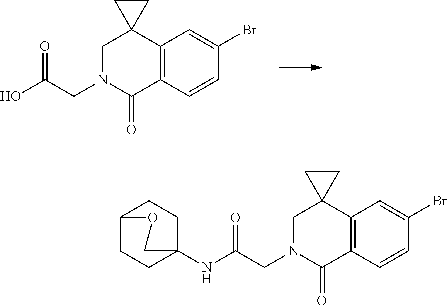 [SMILES: O=C(O)CN1CC2(CC2)c2cc(Br)ccc2C1=O ; O=C(CN1CC2(CC2)c2cc(Br)ccc2C1=O)NC12CCC(CC1)OC2]

To a solution of 2-(6-bromo-1-oxo-spiro[3H-isoquinoline-4,1′-cyclopropane]-2-yl)acetic acid (15 mg, 0.048 mmol) in DMF (1.4 mL) were added 2-oxabicyclo[2.2.2]octan-4-amine (8.6 mg, 0.068 mmol), DIPEA (18.8 mg, 0.15 mmol), and T3P (40 mg, 0.068 mmol, 50% in DMF). The reaction mixture was stirred at 25° C. for 1 h. The reaction mixture was directly purified directly by reverse-phase HPLC. LCMS: m/z=419.4, 421.4 [M+H]⁺. ¹H NMR (400 MHz, DMSO-d₆): δ 7.80 (d, J=8.3 Hz, 1H), 7.62 (s, 1H). 7.51 (dd, J=8.3, 1.9 Hz, 1H), 7.23 (d, J=1.9 Hz, 1H), 4.05 (s, 2H), 3.81 (s, 2H), 3.65-3.64 (m, 1H), 3.40 (s, 2H), 2.02-1.87 (m, 4H), 1.83-1.76 (m, 2H), 1.67-1.60 (m, 2H), 1.15-1.12 (m, 2H), 1.04-1.01 (m, 2H).

The following compounds as shown in Table 1 were, or can be, made via similar procedures as those described above.

| Ex. | Name | NMR | LCMS |
|---|---|---|---|
| 52 | 2-(6-bromo-1- | | m/z = |
| | oxo-spiro[3H- | | 377.3, |
| | isoquinoline- | | 379.3 |
| | 4,1′-cyclopropane]- | | [M + H]⁺ |
| | 2-yl)-N-(3- | | |
| | methylcyclo- | | |
| | butyl)acetamide | | |
| 53 | 2-(6-bromo-1- | ¹H NMR (400 MHz, | m/z = |
| | oxo-spiro[3H- | DMSO-d₆): δ 8.66 (s, | 405.1, |
| | isoquinoline-4,1′- | 1H), 7.81 (d, J = 8.3 Hz, | 407.0 |
| | cyclopropane]- | 1H), 7.52 (dd, J = | [M + H]⁺ |
| | 2-yl)-N-(1- | 8.3, 1.9 Hz, 1H), 7.24 | |
| | methyl-2- | (d, J = 1.9 Hz, 1H), | |
| | oxabicy- | 4.11 (s, 2H), 3.64 (s, 2H), | |
| | clo[2.1.1]hexan-4- | 3.44 (s, 2H), 1.91 | |
| | yl)acetamide | (dd, J = 4.3, 1.6 Hz, 2H), | |
| | | 1.71 (dd, J = 4.3, 1.6 | |
| | | Hz, 2H), 1.34 (s, 3H), | |
| | | 1.15-1.12 (m, 2H), | |
| | | 1.04-1.02 (m, 2H) | |
| 54 | 2-(6-bromo-1- | ¹H NMR (400 MHz, | m/z = |
| | oxo-spiro[3H- | DMSO-d₆): δ 8.43 | 431.3, |
| | isoquinoline-4,1′- | (d, J = 7.4 Hz, 1H), | 433.3 |
| | cyclopropane]- | 7.82-7.80 (m, 1H), 7.52 | [M + H]⁺ |
| | 2-yl)-N-[trans-3- | (dd, J = 8.3, 1.9 Hz, 1H), | |
| | (trifluoromethyl)cy- | 7.24 (d, J = 1.8 Hz, | |
| | clobutyl]acetamide | 1H), 4.36-4.30 (m, 1H), | |
| | | 4.09 (s, 2H), 3.44 (s, | |
| | | 2H), 3.10-3.01 (m, 1H), | |
| | | 2.41-2.25 (m, 4H), | |
| | | 1.15-1.13 (m, 2H), | |
| | | 1.04-1.02 (m, 2H) | |
| 55 | 2-(6-bromo-1- | ¹H NMR (400 MHz, | m/z = |
| | oxo-spiro[3H- | DMSO-d₆): δ 8.44 | 447.3, |
| | isoquinoline-4,1′- | (d, J = 7.2 Hz, 1H), 7.81 | 449.3 |
| | cyclopropane]- | (d, J = 8.3 Hz, 1H), | [M + H]⁺ |
| | 2-yl)-N-[cis- | 7.52 (dd, J = 8.3, 1.9 Hz, | |
| | 3-hydroxy-3- | 1H), 7.24 (d, J = 1.9 | |
| | (trifluoromethyl)cy- | Hz, 1H), 6.71-6.61 (m, | |
| | clobutyl]acetamide | 1H), 4.09 (s, 2H), | |
| | | 3.96-3.90 (m, 1H), | |
| | | 3.44 (s, 2H), 2.74-2.68 | |
| | | (m, 2H), 2.21-2.14 | |
| | | (m, 2H), 1.15-1.12 (m, | |
| | | 2H), 1.04-1.02 (m, 2H) | |
| 56 | N-(1-bicyclo[2.2. | ¹H NMR (400 MHz, | m/z = |
| | 2]octanyl)-2-(6- | DMSO-d₆): δ 7.80 | 417.4, |
| | bromo-1-oxo- | (d, J = 8.3 Hz, 1H), | 419.4 |
| | spiro[3H- | 7.52-7.49 (m, 1H), 7.40- | [M + H]⁺ |
| | isoquinoline-4,1′- | 7.37 (m, 1H), 7.23 (q, | |
| | cyclopropane]- | J = 2.7 Hz, 1H), 4.02 | |
| | 2-yl)acetamide | (s, 2H), 3.40 (s, 2H), | |
| | | 1.78-1.74 (m, 6H), 1.59- | |
| | | 1.55 (m, 6H), 1.52-1.48 | |
| | | (m, 1H), 1.14-1.11 | |
| | | (m, 2H), 1.04-1.01 (m, 2H) | |
| 57 | 2-(6-bromo-1- | ¹H NMR (400 MHz, | m/z = |
| | oxo-spiro[3H- | DMSO-d₆): δ 7.79 | 433.3, |
| | isoquinoline-4,1′- | (d, J = 8.3 Hz, 1H), 7.51 | 435.3 |
| | cyclopropane]- | (dd, J = 8.3, 1.9 Hz, | [M + H]⁺ |
| | 2-yl)-N-(4- | 1H), 7.44 (s, 1H), 7.23 | |
| | hydroxy-1- | (d, J = 1.9 Hz, 1H), | |
| | bicyclo[2.2.2]oc- | 4.26 (s, 1H), 4.01 (s, 2H), | |
| | tanyl)acetamide | 3.39 (s, 2H), 1.90- | |
| | | 1.86 (m, 6H), 1.58-1.54 | |
| | | (m, 6H), 1.14-1.11 | |
| | | (m, 2H), 1.03-1.02 (m, 2H) | |
| 58 | 2-(6-bromo-1- | | m/z = |
| | oxo-spiro[3H- | | 407.4, |
| | isoquinoline-4,1′- | | 409.3 |
| | cyclopropane]- | | [M + H]⁺ |
| | 2-yl)-N-(3- | | |
| | hydroxycyclo- | | |
| | hexyl)acetamide | | |
| 59 | 2-(6-bromo-1- | ¹H NMR (400 MHz, | m/z = |
| | oxo-spiro[3H- | CDCl₃): δ 7.90 (dd, J = | 406.4, |
| | isoquinoline-4,1′- | 8.3, 2.2 Hz, 1H), | 408.3 |
| | cyclopropane]- | 7.45-7.42 (m, 1H), 7.41-7.40 | [M + H]⁺ |
| | 2-yl)-N-(1- | (m, 1H), 6.98-6.97 (m, | |
| | methyl-5-oxo- | 1H), 4.53-4.48 (m, | |
| | pyrrolidin-3- | 1H), 4.21-4.09 (m, 2H), | |
| | yl)acetamide | 3.72-3.67 (m, 1H), | |
| | | 3.52-3.42 (m, 2H), | |
| | | 3.25-3.21 (m, 1H), 2.76- | |
| | | 2.74 (m, 3H), 2.74-2.68 | |
| | | (m, 1H), 2.31-2.25 | |
| | | (m, 1H), 1.14-1.08 (m, 2H), | |
| | | 1.05-0.98 (m, 2H) | |
| 60 | 2-(6-bromo-1- | | m/z = |
| | oxo-spiro[3H- | | 429.2, |
| | isoquinoline-4,1′- | | 431.2 |
| | cyclopropane]- | | [M + H]⁺ |
| | 2-yl)-N-[3- | | |
| | (difluoromethoxy)cy- | | |
| | clobutyl]acetamide | | |
| 61 | 2-(6-bromo-1- | ¹H NMR (400 MHz, | m/z = |
| | oxo-spiro[3H- | CDCl₃): δ 7.95 (d, J = | 405.3, |
| | isoquinoline-4,1′- | 8.3 Hz, 1H), 7.46 (dd, | 407.3 |
| | cyclopropane]- | J = 8.3, 1.9 Hz, 1H), | [M + H]⁺ |
| | 2-yl)-N-(2- | 7.00 (d, J = 1.7 Hz, 1H), | |
| | oxaspiro[3.3]heptan- | 6.54 (d, J = 6.5 Hz, | |
| | 6-yl)acetamide | 1H), 4.70 (s, 2H), 4.57 (s, | |
| | | 2H), 4.19-4.12 (m, | |
| | | 1H), 4.13-4.10 (m, 2H), | |
| | | 3.45 (s, 2H), 2.69- | |
| | | 2.63 (m, 2H), 2.07-2.02 | |
| | | (m, 2H), 1.13-1.09 | |
| | | (m, 2H), 1.03-0.99 (m, 2H) | |
| 62 | 2-(6-bromo-1- | ¹H NMR (400 MHz, | m/z = |
| | oxo-spiro[3H- | CDCl₃): δ 7.93 (d, J = | 434.4, |
| | isoquinoline-4,1′- | 8.3 Hz, 1H), 7.45 (dd, | 436.3 |
| | cyclopropane]- | J = 8.3, 1.9 Hz, 1H), | [M + H]⁺ |
| | 2-yl)-N-(1- | 6.99 (d, J = 1.8 Hz, 1H), | |
| | ethyl-6-oxo-3- | 6.88 (d, J = 7.2 Hz, | |
| | piperidyl)acetamide | 1H), 4.27-4.21 (m, 1H), | |
| | | 4.20-4.10 (m, 2H), | |
| | | 3.53-3.49 (m, 2H), | |
| | | 3.46-3.43 (m, 1H), 3.42- | |
| | | 3.36 (m, 1H), 3.35-3.28 | |
| | | (m, 1H), 3.17-3.12 | |
| | | (m, 1H), 2.53-2.41 (m, | |
| | | 2H), 2.03-1.95 (m, | |
| | | 1H), 1.88-1.78 (m, 1H), | |
| | | 1.15-1.09 (m, 2H), | |
| | | 1.09-1.05 (m, 3H), | |
| | | 1.04-0.99 (m, 2H) | |
| 63 | 2-(6-bromo-1- | ¹H NMR (400 MHz, | m/z = |
| | oxo-spiro[3H- | CDCl₃): δ 7.92 (d, J = | 381.3, |
| | isoquinoline-4,1′- | 8.3 Hz, 1H), 7.43 (dd, | 383.3 |
| | cyclopropane]- | J = 8.3, 1.9 Hz, 1H), | [M + H]⁺ |
| | 2-yl)-N-(2- | 7.00-6.96 (m, 2H), 4.20 | |
| | hydroxy-2-methyl- | (s, 2H), 3.51 (s, 2H), | |
| | propyl)acetamide | 3.27 (d, J = 6.1 Hz, 2H), | |
| | | 1.20 (s, 6H), 1.12- | |
| | | 1.05 (m, 4H) | |
| 64 | 2-(6-bromo-1- | ¹H NMR (400 MHz, | m/z = |
| | oxo-spiro[3H- | CD₃OD): δ 7.88 (d, J = | 393.4, |
| | isoquinoline-4,1′- | 8.4 Hz, 1H), 7.49 (dd, | 395.3 |
| | cyclopropane]- | J = 8.3, 1.9 Hz, 1H), | [M + H]⁺ |
| | 2-yl)-N-[cis-3- | 7.17 (d, J = 1.9 Hz, 1H), | |
| | (hydroxymethyl)cy- | 4.26-4.18 (m, 3H), | |
| | clobutyl]acetamide | 3.51 (d, J = 5.3 Hz, 4H), | |
| | | 2.42-2.35 (m, 2H), | |
| | | 2.23-2.14 (m, 1H), | |
| | | 1.78-1.70 (m, 2H), 1.16- | |
| | | 1.08 (m, 4H) | |
| 65 | 2-(6-bromo-1- | ¹H NMR (400 MHz, | m/z = |
| | oxo-spiro[3H- | CD₃OD): δ 7.89 (d, J = | 388.2, |
| | isoquinoline-4,1′- | 8.3 Hz, 1H), 7.51 (dd, | 390.2 |
| | cyclopropane]- | J = 8.3, 1.9 Hz, 1H), | [M + H]⁺ |
| | 2-yl)-N-(3- | 7.19 (d, J = 1.9 Hz, 1H), | |
| | cyanocyclo- | 4.65-4.56 (m, 1H), | |
| | butyl)acetamide | 4.22 (s, 2H), 3.52 (s, | |
| | | 2H), 3.23 (dd, J = 5.5, | |
| | | 4.1 Hz, 1H), 2.68-2.61 | |
| | | (m, 2H), 2.52-2.45 (m, | |
| | | 2H), 1.17-1.09 (m, 4H) | |
| 66 | 6-bromo-2-[2- | ¹H NMR (400 MHz, | m/z = |
| | [3-(1-hydroxy-1- | CDCl₃): δ 7.98 (d, J = | 421.4, |
| | methyl- | 8.3 Hz, 1H), 7.44 (dt, | 423.3 |
| | ethyl)pyrrolidin- | J = 8.3, 2.1 Hz, 1H), | [M + H]⁺ |
| | 1-yl]-2- | 6.99 (t, J = 1.5 Hz, 1H), | |
| | oxo-ethyl]spiro[3H- | 5.32 (s, 2H), 4.39 | |
| | isoquinoline- | (dd, J = 15.9, 2.8 Hz, | |
| | 4,1′-cyclopropane]- | 1H), 4.26 (dd, J = 15.9, | |
| | 1-one | 8.2 Hz, 1H), 3.83-3.26 | |
| | | (m, 5H), 2.42-2.18 (m, | |
| | | 1H), 2.08-1.73 (m, 2H), | |
| | | 1.29-1.27 (m, 6H), | |
| | | 1.11-1.09 (m, 4H) | |
| 67 | 2-(6-bromo-1- | ¹H NMR (400 MHz, | m/z = |
| | oxo-spiro[3H- | CDCl₃): δ 7.98 (d, J = | 399.3, |
| | isoquinoline-4,1′- | 8.3 Hz, 1H), 7.49 (dd, | 401.3 |
| | cyclopropane]- | J = 8.3, 1.9 Hz, 1H), | [M + H]⁺ |
| | 2-yl)-N-(3,3- | 7.02 (d, J = 1.8 Hz, | |
| | difluorocyclo- | 1H), 6.80-6.76 (m, 1H), | |
| | butyl)acetamide | 4.29-4.21 (m, 1H), 4.16 | |
| | | (s, 2H), 3.50 (s, 2H), | |
| | | 3.06-2.95 (m, 2H), | |
| | | 2.56-2.44 (m, 2H), 1.17- | |
| | | 1.14 (m, 2H), 1.06-1.03 | |
| | | (m, 2H) | |
| 68 | 6-bromo-2-[2- | ¹H NMR (400 MHz, | m/z = |
| | (3-hydroxy-3- | CDCl₃): δ 7.95 (dd, J = | 393.4, |
| | methyl-pyrrolidin- | 8.3, 5.2 Hz, 1H), 7.44 | 395.3 |
| | 1-yl)-2-oxo- | (dd, J = 8.3, 1.9 Hz, | [M + H]⁺ |
| | ethyl]spiro[3H- | 1H), 6.99 (d, J = 1.9 Hz, | |
| | isoquinoline-4,1′- | 1H), 4.33 (d, J = 8.8 | |
| | cyclopropane]- | Hz, 1H), 4.31-4.22 (m, | |
| | 1-one | 1H), 3.81-3.30 (m, | |
| | | 6H), 2.09-1.82 (m, 2H), | |
| | | 1.46 (d, J = 4.2 Hz, | |
| | | 3H), 1.13-1.06 (m, 4H) | |
| 69 | 2-(6-bromo-4- | ¹H NMR (400 MHz, | m/z = |
| | methyl-1-oxo-3,4- | CDCl₃): δ 7.91 (d, J = | 381.3, |
| | dihydroisoquinolin- | 8.3 Hz, 1H), 7.49 (dd, | 383.2 |
| | 2-yl)-N-(cis- | J = 8.3, 1.8 Hz, 1H), | [M + H]⁺ |
| | 3-hydroxy-3- | 7.39 (d, J = 1.5 Hz, 1H), | |
| | methyl- | 6.70 (d, J = 7.2 Hz, | |
| | cyclobutyl)acetamide | 1H), 4.20-4.08 (m, 2H), | |
| | | 4.01-3.91 (m, 1H), | |
| | | 3.78-3.73 (m, 1H), | |
| | | 3.45-3.40 (m, 1H), 3.19- | |
| | | 3.11 (m, 1H), 2.52-2.47 | |
| | | (m, 2H), 2.05-1.98 | |
| | | (m, 2H), 1.35 (s, 3H), 1.33 | |
| | | (d, J = 7.0 Hz, 3H) | |

### Example 70

### 2-(6-bromo-1-oxo-spiro[3H-isoquinoline-4,1′-cyclopropane]-2-yl)-N-[(3R)-1-cyclobutyl-3-piperidyl]acetamide (70)

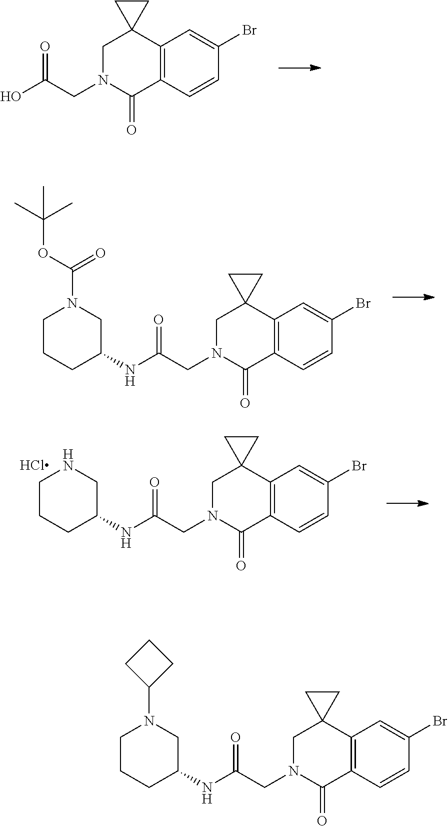

tert-Butyl (3R)-3-[[2-(6-bromo-1-oxo-spiro[3H-isoquinoline-4,1′-cyclopropane]-2-yl)acetyl]amino]piperidine-1-carboxylate: To a solution of 2-(6-bromo-1-oxo-spiro[3H-isoquinoline-4,1′-cyclopropane]-2-yl)acetic acid (73 mg, 0.024 mmol), tert-butyl (3R)-3-aminopiperidine-1-carboxylate (66 mg, 0.033 mmol), DIPEA (92 mg, 0.706 mmol) in DMF (1.4 mL) was added T3P (195 mg, 0.306 mmol, 50% in DMF). The reaction mixture was stirred at 25° C. for 1 h. The reaction mixture was diluted with sat. aq. NaHCO₃ (10 mL) and extracted with EtOAc (3×10 mL). The combined organics were washed with brine (10 mL), dried over anhydrous Na₂SO₄, filtered, and concentrated under reduced pressure. The residue was purified by reverse-phase HPLC. LCMS: m/z=492.4, 494.4 [M+H]⁺.

2-(6-bromo-1-oxo-spiro[3H-isoquinoline-4,1′-cyclopropane]-2-yl)-N-[(3R)-3-piperidyl]acetamide HCl salt: A solution of tert-butyl (3R)-3-[[2-(6-bromo-1-oxo-spiro[3H-isoquinoline-4,1′-cyclopropane]-2-yl)acetyl]amino]piperidine-1-carboxylate (101 mg, 0.20 mmol) in HCl (5 mL, 4 N in dioxane) was stirred at 23° C. for 3 h. The reaction mixture was concentrated under reduced pressure to provide a residue that was used directly. LCMS: m/z=392.3, 394.3 [M+H]⁺.

2-(6-bromo-1-oxo-spiro[3H-isoquinoline-4,1′-cyclopropane]-2-yl)-N-[(3R)-1-cyclobutyl-3-piperidyl]acetamide: To a solution of 2-(6-bromo-1-oxo-spiro[3H-isoquinoline-4,1′-cyclopropane]-2-yl)-N-[(3R)-3-piperidyl]acetamide hydrochloride (30 mg, 0.07 mmol), cyclobutanone (9.8 mg, 0.14 mmol), and acetic acid (8.4 mg, 0.14 mmol) in methanol (1.0 mL) was added sodium cyanoborohydride (7.5 mg, 0.12 mmol). The reaction mixture was stirred at 23° C. for 18 h. The reaction mixture was diluted with sat. aq. NaHCO₃ (10 mL) and extracted with EtOAc (3×10 mL). The combined organics were washed with brine (15 mL), dried over anhydrous Na₂SO₄, filtered, and concentrated under reduced pressure. The residue was purified by reverse-phase HPLC. LCMS: m/z=446.3 448.3 [M+H]⁺. ¹H NMR (400 MHz, DMSO-d₆): δ 8.16 (s, 1H), 7.81 (d, J=8.3 Hz, 1H), 7.54-7.50 (m, 1H), 7.24 (d, J=1.9 Hz, 1H), 4.09 (s, 2H), 3.71-3.65 (m, 1H), 3.44 (s, 2H), 2.69-2.59 (m, 2H), 1.95-1.90 (m, 2H), 1.80-1.55 (m, 8H), 1.44-1.40 (m, 2H), 1.26-1.18 (m, 1H), 1.15-1.13 (m, 2H), 1.05-1.03 (m, 2H).

### Example 71

### methyl (3R)-3-[[2-(6-bromo-1-oxo-spiro[3H-isoquinoline-4,1′-cyclopropane]-2-yl)acetyl]amino]piperidine-1-carboxylate (71)

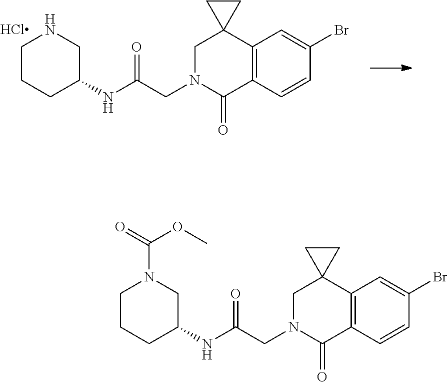 [SMILES: O=C(CN1CC2(CC2)c2cc(Br)ccc2C1=O)N[C@@H]1CCCNC1 ; COC(=O)N1CCC[C@@H](NC(=O)CN2CC3(CC3)c3cc(Br)ccc3C2=O)C1]

To a solution of 2-(6-bromo-1-oxo-spiro[3H-isoquinoline-4,1′-cyclopropane]-2-yl)-N-[(3R)-3-piperidyl]acetamide HCl salt (20 mg, 0.05 mmol) in THF (1.0 mL) were added methyl chloroformate (8.8 mg, 0.09 mmol) and DIPEA (0.02 mL, 0.14 mmol). The reaction mixture was stirred at 23° C. for 3 h. The reaction mixture was concentrated under reduced pressure. The residue was purified by reverse-phase HPLC. LCMS: m/z=450.4, 452.4 [M+H]⁺. ¹H NMR (400 MHz, DMSO-d₆): δ 8.01-7.99 (m, 1H), 7.81 (d, J=8.3 Hz, 1H), 7.54-7.50 (m, 1H), 7.25 (d, J=1.9 Hz, 1H), 4.12-4.09 (m, 2H), 3.81-3.75 (m, 1H), 3.68-3.63 (m, 2H), 3.59-3.55 (m, 3H), 3.44 (s, 2H), 2.98-2.91 (m, 1H), 2.84-2.68 (m, 1H), 1.82-1.65 (m, 2H), 1.43-1.36 (m, 2H), 1.16-1.13 (m, 2H), 1.05-1.03 (m, 2H).

### Example 72

### 2-(6-bromo-1-oxospiro[3H-isoquinoline-4,1′-cyclopropane]-2-yl)-N-(5-cyanopyridin-2-yl)acetamide (72)

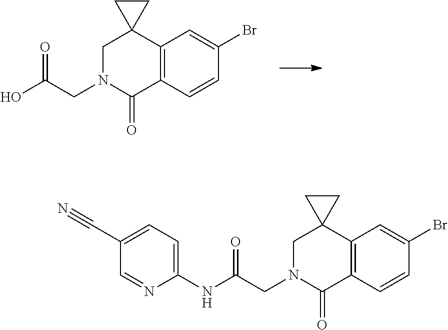 [SMILES: O=C(O)CN1CC2(CC2)c2cc(Br)ccc2C1=O ; N#Cc1ccc(NC(=O)CN2CC3(CC3)c3cc(Br)ccc3C2=O)nc1]

To a solution of 2-(6-bromo-1-oxo-spiro[3H-isoquinoline-4,1′-cyclopropane]-2-yl)acetic acid (27 mg, 0.09 mmol) in MeCN (1.0 mL) were added 2-amino-5-cyanopyridine (14 mg, 0.11 mmol), 1-methylimidazole (28 mg, 0.34 mmol), and chloro-N,N,N′,N′-tetramethylformamidinium hexafluorophosphate (31 mg, 0.11 mmol). The reaction mixture was stirred at 23° C. for 20 h. The reaction mixture was purified directly by reverse-phase HPLC. LCMS: m/z=411.2, 413.2 [M+H]⁺. ¹H NMR (400 MHz, CDCl₃): δ 9.17-9.15 (m, 1H), 8.56 (s, 1H), 8.32-8.30 (m, 1H), 8.02 (d, J=8.3 Hz, 1H), 7.95-7.92 (m, 1H), 7.49-7.46 (m, 1H), 7.01 (s, 1H), 4.36 (s, 2H), 3.52 (s, 2H), 1.18-1.13 (m, 2H), 1.07-1.03 (m, 2H).

The following compounds as shown in Table 1 were, or can be, made via similar procedures as those described above.

| Ex. | Name | NMR | LCMS |
|---|---|---|---|
| 73 | 2-(6-bromo-1-oxo-spiro[3H- | ¹H NMR (400 MHZ, CDCl₃): δ 10.38 (br s, | m/z = |
| | isoquinoline-4,1′-cyclopropane]- | 1H), 7.89 (d, J = 8.3 Hz, 1H), 7.40 (dd, J = | 416.3, |
| | 2-yl)-N-(1-methyl-6-oxo-3- | 8.3, 1.7 Hz, 1H), 7.38-7.35 (m, 1H), 7.12- | 418.2 |
| | pyridyl)acetamide | 7.02 (m, 2H), 6.96 (d, J = 1.8 Hz, 1H), 4.43 | [M + H]⁺ |
| | | (d, J = 0.5 Hz, 2H), 3.57 (s, 3H), 3.49 (s, | |
| | | 2H), 1.09-1.07 (m, 2H), 1.03 (dd, J = 7.5, | |
| | | 2.3 Hz, 2H) | |
| 74 | 2-(6-bromo-4-methyl-1-oxo-3,4- | ¹H NMR (400 MHZ, acetone-d₆): δ 10.03 | m/z = |
| | dihydroisoquinolin-2-yl)-N- | (br s, 1H), 8.76-8.75 (m, 2H), 7.90 (d, J = | 375.1, |
| | pyrimidin-2-yl-acetamide | 8.9 Hz, 1H), 7.61-7.58 (m, 1H), 7.56-7.54 | 377.1 |
| | | (m, 1H), 7.31-7.29 (m, 1H), 4.91-4.73 (m, | [M + H]⁺ |
| | | 2H), 3.91-3.86 (m, 1H), 3.56-3.50 (m, 1H), | |
| | | 3.32-3.28 (m, 1H), 1.40 (d, J = 6.9 Hz, 3H) | |
| 75 | 2-(6-bromo-1-oxo-spiro[3H- | ¹H NMR (400 MHZ, CDCl₃): δ 9.26 (nr s, | |
| | isoquinoline-4,1′-cyclopentane]- | 1H), 8.62 (d, J = 4.8 Hz, 2H), 7.98 (d, J = | m/z = |
| | 2-yl)-N-pyrimidin-2-yl- | 8.3 Hz, 1H), 7.45 (dd, J = 8.3, 1.6 Hz, 1H), | 415.2, |
| | acetamide | 7.40 (s, 1H), 7.05-7.03 (m, 1H), 4.70 (s, | 417.2 |
| | | 2H), 3.53 (s, 2H), 1.90-1.86 (m, 4H), 1.84- | [M + H]⁺ |
| | | 1.77 (m, 4H) | |

### Example 76

### 2-[3-methyl-1-oxo-6-(trifluoromethyl)spiro[3H-isoquinoline-4,1′-cyclopropane]-2-yl]-N-pyrimidin-2-ylacetamide (76)

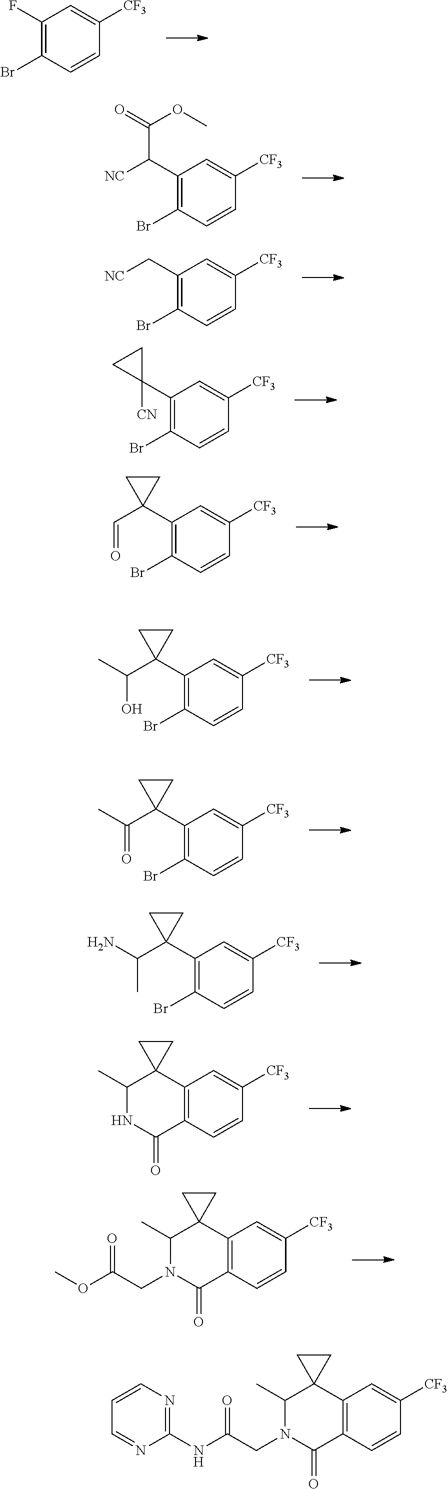

Methyl 2-(2-bromo-5-(trifluoromethyl)phenyl)-2-cyanoacetate: To a solution of 1-bromo-2-fluoro-4-(trifluoromethyl)benzene (25 g, 102.9 mmol) in NMP (250 mL) was added methyl 2-cyanoacetate (10.2 g, 102.9 mmol) and Cs₂CO₃ (83.8 g, 257.2 mmol). The mixture was stirred at 120° C. for 3 h. The reaction mixture was diluted with water (250 mL) and extracted with EtOAc (3×250 mL). The combined organic layers were washed with brine (250 mL), dried over anhydrous Na₂SO₄, filtered, and concentrated under reduced pressure. The residue was purified by silica gel column chromatography. ¹H NMR (400 MHz, CDCl₃): δ 7.84-7.77 (m, 2H), 7.56 (dd, J=1.6, 8.4 Hz, 1H), 5.28 (s, 1H), 3.88 (s, 3H).

2-(2-bromo-5-(trifluoromethyl)phenyl)acetonitrile: To a solution of methyl 2-(2-bromo-5-(trifluoromethyl)phenyl)-2-cyanoacetate (17.8 g, 55.0 mmol) in DMSO (200 mL) and water (50 mL) was added NaCl (3.22 g, 55.0 mmol). The mixture was stirred at 120° C. for 16 h. The reaction mixture was diluted with water (500 mL) and extracted with EtOAc (3×300 mL). The combined organic layers were washed with brine (300 mL), dried over anhydrous Na₂SO₄, filtered, and concentrated under reduced pressure. The residue was purified by silica gel column chromatography. ¹H NMR (400 MHz, CDCl₃): δ 7.80-7.75 (m, 2H), 7.50 (dd, J=1.6, 8.0 Hz, 1H), 3.91 (s, 2H).

1-(2-bromo-5-(trifluoromethyl)phenyl)cyclopropanecarbonitrile: To a solution of NaH (3.60 g, 90.1 mmol. 60% purity) in DMSO (100 mL) was added 2-(2-bromo-5-(trifluoromethyl)phenyl)acetonitrile (10.3 g, 39.2 mmol) and 1-bromo-2-chloro-ethane (6.74 g, 47.0 mmol) at 0° C. The mixture was stirred at 20° C. for 2 h. The reaction mixture was diluted with water (300 mL) and extracted with EtOAc (3×150 mL). The combined organic layers were washed with brine (150 mL), dried over anhydrous Na₂SO₄, filtered and concentrated under reduced pressure. The residue was purified by silica gel column chromatography. ¹H NMR (400 MHz, CDCl₃): δ 7.78 (d, J=8.0 Hz, 1H), 7.60 (s, 1H), 7.49 (dd, J=1.6, 8.0 Hz, 1H), 1.90-1.79 (m, 2H), 1.46-1.35 (m, 2H).

1-(2-bromo-5-(trifluoromethyl)phenyl)cyclopropanecarbaldehyde: To a solution of 1-(2-bromo-5-(trifluoromethyl)phenyl)cyclopropanecarbonitrile (9.0 g, 31.0 mmol) in THF (100 mL) was added DIBAL (1 M in toluene, 62.1 mL) at 0° C. The mixture was stirred at 20° C. for 12 h. The reaction mixture was diluted with water (200 mL) and extracted with EtOAc (3×150 mL). The combined organic layers were washed with brine (150 mL), dried over anhydrous Na₂SO₄, filtered and concentrated under reduced pressure. The residue was purified by silica gel column chromatography. ¹H NMR (400 MHz, CDCl₃): δ 9.09 (s, 1H). 7.79-7.74 (m, 1H), 7.51 (s. 1H), 7.49-7.44 (m, 1H), 1.86-1.72 (m, 2H), 1.54-1.41 (m, 2H).

1-(1-(2-bromo-5-(trifluoromethyl)phenyl)cyclopropyl)ethanol: To a solution of 1-(2-bromo-5-(trifluoromethyl)phenyl)cyclopropanecarbaldehyde (6.0 g, 20.5 mmol) in THF (60 mL) at 0° C. was added MeMgBr (3 M in Et₂O, 6.82 mL). The reaction mixture was stirred at 20° C. for 1 h. The mixture was diluted with water (100 mL) and extracted with EtOAc (3×50 mL). The combined organics were washed with brine (50 mL), dried over anhydrous Na₂SO₄, filtered, and concentrated under reduced pressure. The residue was purified by silica gel chromatography. ¹H NMR (400 MHz, CDCl₃): δ 7.68 (d, J=8.0 Hz, 1H), 7.59 (d, J=1.6 Hz, 1H), 7.36 (dd, J=1.6, 8.0 Hz, 1H), 3.85 (m, 1H), 1.14 (d, J=6.4 Hz, 3H), 1.12-1.03 (m, 2H), 0.96-0.81 (m, 2H).

1-(1-(2-bromo-5-(trifluoromethyl)phenyl)cyclopropyl)ethanone: To a solution of 1-(1-(2-bromo-5-(trifluoromethyl)phenyl)cyclopropyl)ethanol (2.5 g, 8.1 mmol) in DCM (30 mL) was added DMP (6.9 g, 16.2 mmol) at 0° C. The mixture was stirred at 35° C. for 1 h. The mixture was diluted with DCM (20 mL) and washed with H₂O (20 mL). The organic layer was dried over anhydrous Na₂SO₄, filtered, and concentrated under reduced pressure. The residue was purified by silica gel chromatography. ¹H NMR (400 MHz, CDCl₃): δ 7.76 (d, J=8.4 Hz, 1H), 7.62 (d, J=2.0 Hz, 1H), 7.46 (dd, J=8.0, 2.0 Hz, 1H), 2.03 (s, 3H), 1.90-1.76 (m, 2H), 1.31-1.19 (m, 2H).

1-(1-(2-bromo-5-(trifluoromethyl)phenyl)cyclopropyl)ethanamine: To a solution of 1-(1-(2-bromo-5-(trifluoromethyl)phenyl)cyclopropyl)ethanone (1.8 g, 5.9 mmol) in MeOH (20 mL) was added NH₄OAc (3.16 g, 41.0 mmol). Then the mixture was stirred at 20° C. for 1 h. NaBH₃CN (1.84 g, 29.3 mmol) was added at 20° C. Then the mixture was stirred at 80° C. for 16 h. The mixture was quenched with water (10 mL) and concentrated under reduced pressure. The residue was purified by reverse-phase HPLC. ¹H NMR (400 MHz, CDCl₃): δ 7.69 (d, J=8.4 Hz, 1H), 7.57 (s, 1H), 7.36 (dd, J=8.0, 1.6 Hz, 1H), 3.05 (m, 1H), 1.12-1.03 (m, 5H), 0.90-0.76 (m, 2H).

3-methyl-6-(trifluoromethyl)spiro[2,3-dihydroisoquinoline-4,1′-cyclopropane]-1-one: To a solution of 1-(1-(2-bromo-5-(trifluoromethyl)phenyl)cyclopropyl)ethanamine (100 mg, 0.32 mmol) in toluene (10 mL) was added Pd(dba)₂ (9 mg, 0.02 mmol), Na₂CO₃ (103 mg, 0.97 mmol) and bis(1-adamantyl)-butyl-phosphane (12 mg, 0.03 mmol) at 20° C. The suspension was degassed under vacuum and purged with CO several times. The mixture was stirred at 80° C. for 16 h under an atmosphere of CO (30 psi). The mixture was concentrated under reduced pressure. The residue was purified by silica gel chromatography. ¹H NMR (400 MHz, CDCl₃): δ 8.22 (d, J=8.0 Hz, 1H), 7.61-7.54 (m, 1H), 7.13 (s, 1H), 6.47 (br s, 1H), 3.15 (m, 1H), 1.48-1.39 (m, 1H), 1.23 (d, J=6.8 Hz, 3H), 1.19-1.11 (m, 1H), 0.98 (m, 1H), 0.84 (m, 1H).

Methyl 2-[3-methyl-1-oxo-6-(trifluoromethyl)spiro[3H-isoquinoline-4,1′-cyclopropane]-2-yl]acetate: To a solution of 3-methyl-6-(trifluoromethyl)spiro[2,3-dihydroisoquinoline-4,1′-cyclopropane]-1-one (70 mg, 0.27 mmol) in DMF (2 mL) was added NaH (12.1 mg, 0.3 mmol, 60% purity) at 0° C. The mixture was stirred at 0° C. for 0.5 h. Then a solution of methyl 2-bromoacetate (62.9 mg, 0.41 mmol) was added to above the mixture. The mixture was stirred at 20° C. for 2 h. The mixture was quenched with H₂O (5 mL) and extracted with EtOAc (3×5 mL). The organic layer was washed with brine (3×5 mL), H₂O (5 mL), dried over anhydrous Na₂SO₄, filtered and concentrated under reduced pressure. The residue was purified by silica gel chromatography. LCMS: m/z=328.1 [M+H]⁺.

2-(3′-methyl-1′-oxo-6′-(trifluoromethyl)-1′H-spiro[cyclopropane-1,4′-isoquinolin]-2′(3′H)-yl)-N-(pyrimidin-2-yl)acetamide: To a solution of pyrimidin-2-amine (29.06 mg, 0.32 mmol), methyl 2-[3-methyl-1-oxo-6-(trifluoromethyl)spiro[3H-isoquinoline-4,1′-cyclopropane]-2-yl]acetate (50 mg, 0.15 mmol) in THF (1 mL) and toluene (2 mL) was added AlMe₃ (2 M in toluene, 0.15 mL). The mixture was stirred at 90° C. for 4 h. The mixture was quenched with water (2 mL) and extracted with EtOAc (2×5 mL). The organic layer was washed with brine (3×5 mL), H₂O (5 mL), dried over anhydrous Na₂SO₄, filtered and concentrated under reduced pressure. The residue was purified by reverse-phase preparative HPLC. LCMS: m/z=391.0 [M+H]⁺. ¹H NMR (400 MHz, CDCl₃): δ 9.05 (br s, 1H), 8.64 (br s, 2H), 8.26 (d, J=8.0 Hz, 1H), 7.59 (d, J=8.4 Hz, 1H), 7.14 (s, 1H), 7.07 (br s, 1H), 5.08 (br d, J=16.4 Hz, 1H), 4.15 (br d, J=16.4 Hz, 1H), 3.05 (q, J=6.4 Hz, 1H), 1.65-1.59 (m, 1H), 1.37-1.32 (m, 1H), 1.27 (d, J=6.4 Hz, 3H), 0.94-0.88 (m, 1H), 0.81-0.74 (m. 1H).

### Example 77

### 1-(6-bromo-1-oxospiro[3H-isoquinoline-4,1′-cyclopropane]-2-yl)-N-pyrimidin-2-cyclopropane-1-carboxamide (77)

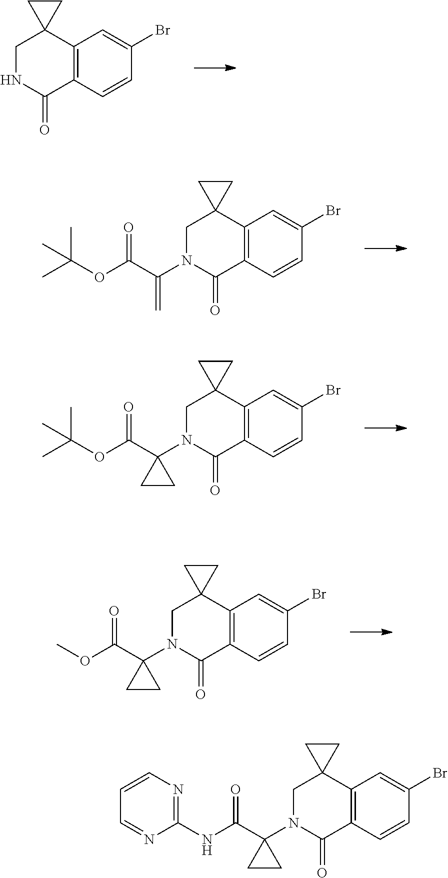 [SMILES: O=C1NCC2(CC2)c2cc(Br)ccc21 ; C=C(C(=O)OC(C)(C)C)N1CC2(CC2)c2cc(Br)ccc2C1=O ; CC(C)(C)OC(=O)C1(N2CC3(CC3)c3cc(Br)ccc3C2=O)CC1 ; COC(=O)C1(N2CC3(CC3)c3cc(Br)ccc3C2=O)CC1 ; O=C1c2ccc(Br)cc2C2(CC2)CN1C1(C(=O)Nc2ncccn2)CC1]

tert-butyl 2-(6-bromo-1-oxospiro[3H-isoquinoline-4,1′-cyclopropane]-2-yl)prop-2-enoate: To a solution of PPh₃ (624 mg, 2.38 mmol) and 6-bromospiro[2,3-dihydroisoquinoline-4,1′-cyclopropane]-1-one (600 mg, 2.38 mmol) in DCM (20 mL) at 0° C. was added a solution of tert-butyl propiolate (300 mg, 2.38 mmol) in DCM (2 mL) dropwise. The mixture was stirred at 0° C. for 5 min and then warmed to 20° C. stirred for a further 16 h. The mixture was concentrated under reduced pressure. The residue was purified by silica gel column chromatography. ¹H NMR (400 MHz, CDCl₃): δ 7.99 (d, J=8.4 Hz, 1H), 7.44 (dd, J=8.4, 2.0 Hz, 1H), 7.00 (d, J=2.0 Hz, 1H), 6.03 (s, 1H), 5.48 (s, 1H), 3.58 (s, 2H), 1.51 (s, 9H), 1.18-1.12 (m, 2H), 1.11-1.05 (m, 2H).

tert-butyl 1-(6-bromo-1-oxospiro[3H-isoquinoline-4,1′-cyclopropane]-2-yl)cyclopropane-1-carboxylate: To a mixture of trimethylsulfoxonium iodide (381.70 mg, 1.73 mmol) in DMSO (5 mL) was added NaH (69.4 mg, 1.73 mmol, 60% purity) at 20° C. Then the mixture was stirred at 20° C. for 40 min. A solution tert-butyl 2-(6-bromo-1-oxospiro[3H-isoquinoline-4,1′-cyclopropane]-2-yl)prop-2-enoate (0.328 g, 0.87 mmol) in DMSO (1 mL) was added to the above mixture at 20° C. Then the mixture was stirred at 20° C. for 1 h. The mixture was poured into H₂O (10 mL) and extracted with DCM (3×10 mL). The organic layer was concentrated under reduced pressure. The residue was purified by silica gel column chromatography. LCMS: m/z=336.0, 338.0 [M-55]⁺.

Methyl 1-(6-bromo-1-oxospiro[3H-isoquinoline-4,1′-cyclopropane]-2-yl)cyclopropane-1-carboxylate: A solution of tert-butyl 1-(6-bromo-1-oxospiro[3H-isoquinoline-4,1′-cyclopropane]-2-yl)cyclopropane-1-carboxylate (180 mg, 0.46 mmol) in HCl (1 mL, 4 M in MeOH) was stirred at 20° C. for 32 h. The mixture was concentrated under reduced pressure. The residue was purified by silica gel column chromatography. ¹H NMR (400 MHz, CDCl₃): δ 7.98 (d, J=8.4 Hz, 1H), 7.42 (dd, J=8.4, 2.0 Hz, 1H), 6.95 (d, J=1.6 Hz, 1H), 3.69 (s, 3H), 3.45 (br s, 2H), 1.71 (br s, 2H), 1.33-1.17 (m, 2H), 1.15-1.02 (m, 2H), 1.00-0.90 (m, 2H).

1-(6-bromo-1-oxospiro[3H-isoquinoline-4,1′-cyclopropane]-2-yl)-N-pyrimidin-2-ylcyclopropane-1-carboxamide: To a solution of methyl 1-(6-bromo-1-oxospiro[3H-isoquinoline-4,1′-cyclopropane]-2-yl)cyclopropane-1-carboxylate (87 mg, 0.25 mmol) and pyrimidin-2-amine (35 mg, 0.37 mmol) in THF (3 mL) and toluene (1 mL) was added AlMe₃ (2 M in toluene, 0.25 mL) at 20° C. The mixture was stirred at 90° C. for 6 h. The mixture was poured into water (10 mL) and extracted with EtOAc (3×10 mL). The organic layer was dried over anhydrous Na₂SO₄, filtered and concentrated under reduced pressure. The residue was purified by reverse-phase preparative HPLC. LCMS: m/z=413.0, 415.0 [M+H]⁺. ¹H NMR (400 MHz, CDCl₃): δ 9.27 (br s, 1H), 8.61 (d, J=4.8 Hz, 2H), 8.04 (d, J=8.4 Hz, 1H), 7.48 (br d, J=8.4 Hz, 1H), 7.06-6.93 (m, 2H), 3.49 (s, 2H), 1.87-1.83 (m, 2H), 1.38-1.22 (m, 2H), 1.18 (br s, 2H), 1.05 (br s, 2H).

### Examples 78 and 79

### 2-[(2′s,4r)-6-bromo-2′-fluoro-1-oxospiro[3H-isoquinoline-4,1′-cyclopropane]-2-yl]-N-(5-fluoropyrimidin-2-yl)acetamide (78 and 79)

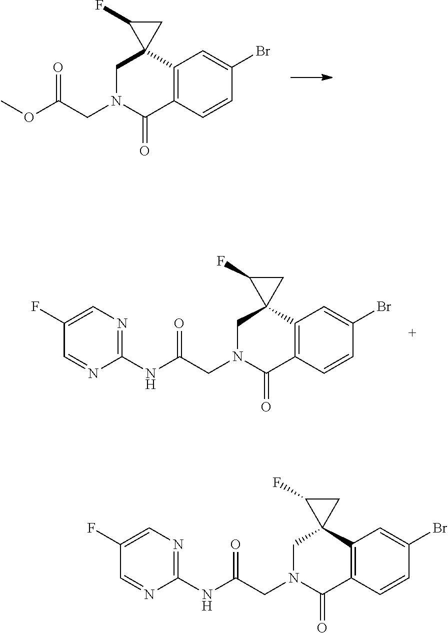

A mixture of 5-fluoropyrimidin-2-amine (59 mg, 0.53 mmol) and methyl 2-[(2′s,4r)-6-bromo-2′-fluoro-1-oxospiro[3H-isoquinoline-4,1′-cyclopropane]-2-yl]acetate (90 mg, 0.26 mmol, Int. 31), and DABAL-Me₃ (202 mg, 0.79 mmol) in toluene (3.0 mL) was stirred at 60° C. for 12 h. The reaction mixture was poured into water (10 mL) and the mixture was extracted with EtOAc (3×10 mL). The combined organic layers were washed with brine (10 mL), dried over anhydrous Na₂SO₄, filtered, and concentrated under reduced pressure. The residue was purified by reverse-phase preparative HPLC and further purified by chiral SFC (Column: Regis(S,S) Whelk-O1 (250 mm×30 mm, 10 μm particle size); Mobile Phase: A: CO₂, B: 0.1% NH₄OH in EtOH; Gradient: 50% B isocratic; Flow Rate: 70 g/min; Detection Wavelength: 220 nm; Column Temperature: 35° C.; System Back Pressure: 150 bar) to provide:

2-[(2′s,4r)-6-bromo-2′-fluoro-1-oxospiro[3H-isoquinoline-4,1′-cyclopropane]-2-yl]-N-(5-fluoropyrimidin-2-yl)acetamide (78, first eluting isomer): LCMS: m/z=423.0, 425.0 [M+H]⁺. ¹H NMR (400 MHz, CDCl₃): δ 9.00 (br s, 1H), 8.49 (s, 2H), 8.05 (d, J=8.4 Hz. 1H), 7.52 (dd, J=1.6, 8.4 Hz, 1H), 6.85 (s, 1H), 4.96 (d, J=15.0 Hz, 1H), 4.73-4.50 (m, 1H), 4.38 (d, J=16.0 Hz, 1H), 4.18 (d, J=12.8 Hz, 1H), 3.53 (d, J=12.8 Hz, 1H), 1.69-1.54 (m, 1H), 1.48-1.35 (m, 1H);

2-[(2′s,4r)-6-bromo-2′-fluoro-1-oxospiro[3H-isoquinoline-4,1′-cyclopropane]-2-yl]-N-(5-fluoropyrimidin-2-yl)acetamide (79, second eluting isomer): LCMS: m/z=422.9, 424.9 [M+H]⁺. ¹H NMR (400 MHz, CDCl₃): δ 8.98 (br s, 1H), 8.49 (s, 2H), 8.05 (d, J=8.0 Hz, 1H), 7.52 (dd, J=2.0, 8.4 Hz, 1H), 6.85 (d, J=1.6 Hz, 1H), 4.96 (d, J=14.4 Hz, 1H), 4.73-4.51 (m, 1H), 4.38 (d, J=16.8 Hz, 1H), 4.18 (dd, J=2.0, 12.8 Hz, 1H), 3.53 (d, J=12.8 Hz, 1H), 1.65-1.57 (m, 1H), 1.47-1.36 (m, 1H).

Example 78 has been identified as a single enantiomer, 2-[(2′S,4R)-6-bromo-2′-fluoro-1-oxospiro[3H-isoquinoline-4,1′-cyclopropane]-2-yl]-N-(5-fluoropyrimidin-2-yl)acetamide, as shown in Table 1A.

Example 79 has been identified as a single enantiomer, 2-[(2′R,4S)-6-bromo-2′-fluoro-1-oxospiro[3H-isoquinoline-4,1′-cyclopropane]-2-yl]-N-(5-fluoropyrimidin-2-yl)acetamide, as shown in Table 1A.

### Example 80

### 2-[(2′r,4r)-6-bromo-2′-fluoro-1-oxospiro[3H-isoquinoline-4,1′-cyclopropane]-2-yl]-N-(5-fluoropyrimidin-2-yl)acetamide (80)

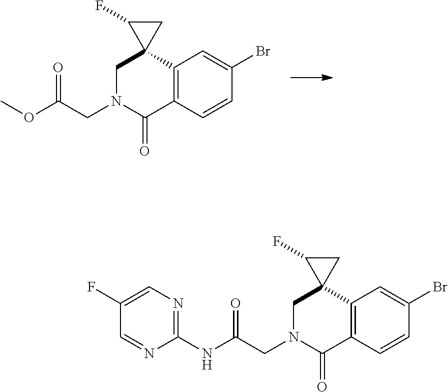

A mixture of 5-fluoropyrimidin-2-amine (33 mg, 0.29 mmol), methyl 2-[(2′r,4r)-6-bromo-2′-fluoro-1-oxospiro[3H-isoquinoline-4,1′-cyclopropane]-2-yl]acetate (50 mg, 0.15 mmol, Int. 35), and DABAL-Me₃ (112 mg, 0.44 mmol) in toluene (3.0 mL) was stirred at 60° C. for 12 h. The reaction mixture was poured into water (10 mL) and extracted with EtOAc (3×10 mL). The combined organic layers were washed with brine (10 mL), dried over anhydrous Na2SO4, filtered, and concentrated under reduced pressure. The residue was purified by reverse-phase preparative HPLC. LCMS: m/z=423.0, 425.0 [M+H]⁺. ¹H NMR (400 MHz, CDCl₃): δ 8.93 (br s, 1H), 8.49 (s, 2H), 8.06 (d, J=8.4 Hz, 1H), 7.55 (dd, J=2.0 Hz, 8.0 Hz, 1H), 7.27-7.26 (m, 1H), 4.98-4.70 (m, 2H), 4.55 (d, J=14.0 Hz, 1H), 4.02 (d, J=12.8 Hz. 1H), 2.84 (d, J=12.8 Hz, 1H), 1.89-1.70 (m, 1H), 1.35-1.09 (m, 1H).

### Example 81

### N-(5-fluoropyrimidin-2-yl)-2-(6-bromo-1-oxospiro[3H-isoquinoline-4,1′-(spiro[2.2]pentan-1-yl)]-2-yl)acetamide (81)

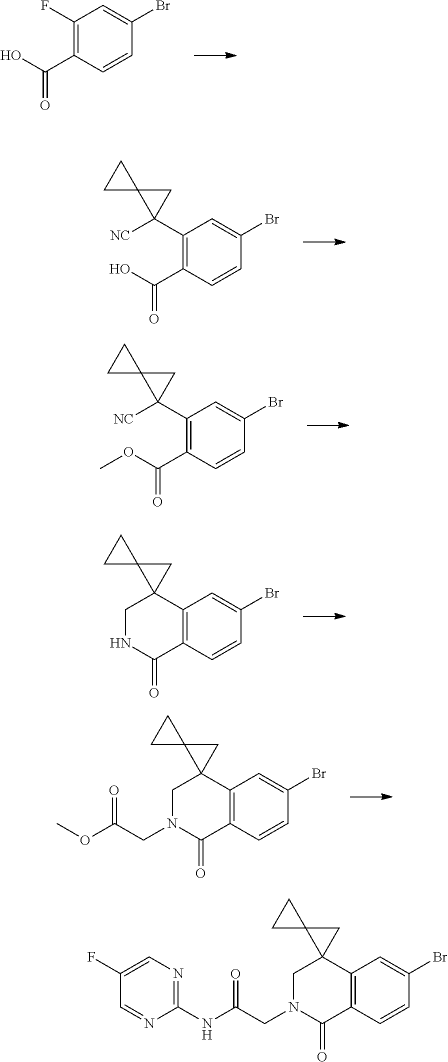

4-bromo-2-(1-cyanospiro[2.2]pentan-1-yl)benzoic acid: To a solution of spiro[2.2]pentane-1-carbonitrile (170 mg, 1.83 mmol), 4-bromo-2-fluorobenzoic acid (100 mg, 0.46 mmol) in THF (2 mL) was added KHMDS (1 M in THF, 1.19 mL) at −40° C. The mixture was stirred at 45° C. for 12 h. The reaction mixture was poured into ice-cold water (20 mL) and extracted with MTBE (3×10 mL). The organics were discarded and the aqueous phase was adjusted to pH=3 with aq. HCl (3 M) and extracted with EtOAc (4×10 mL). The combined organic layers were washed with brine (2×10 mL), dried over anhydrous Na₂SO₄, filtered, and concentrated under reduced pressure. LCMS: m/z=292.0, 290.0 [M−H]⁻

Methyl 4-bromo-2-(1-cyanospiro[2.2]pentan-1-yl)benzoate: To a solution of 4-bromo-2-(1-cyanospiro[2.2]pentan-1-yl)benzoic acid (580 mg, 1.99 mmol). K₂CO₃ (412 mg, 2.98 mmol) in DMF (10 mL) was added CH₃I (310 mg, 2.18 mmol). The mixture was stirred at 20° C. for 3 h. The reaction mixture was poured into ice-cold water (10 mL) and extracted with EtOAc (3×5 mL). The combined organic layers were washed with brine (3×10 mL), dried over anhydrous Na₂SO₄, filtered, and concentrated under reduced pressure. The residue was purified by silica gel column chromatography. LCMS: m/z=306.0, 308.0 [M+H]⁺.

6-bromospiro[2,3-dihydroisoquinoline-4,1′-(spiro[2.2]pentan-1-yl)]-1-one: To a mixture of methyl 4-bromo-2-(1-cyanospiro[2.2]pentan-1-yl)benzoate (190 mg, 0.62 mmol) and dichlorocobalt (81 mg, 0.62 mmol) in MeOH (3 mL) and THF (2 mL) at 0° C. was added NaBH₄ (50 mg, 1.32 mmol). The mixture was stirred at 20° C. for 3 h. The reaction mixture was cooled to 0° C., diluted with sat. aq. NH₄Cl (10 mL), and extracted with DCM (4×5 mL). The combined organic layers were washed with brine (10 mL), dried over anhydrous Na₂SO₄, filtered, and concentrated under reduced pressure. LCMS: m/z=278.0, 280.0 [M+H]⁺.

Methyl 2-(6-bromo-5-fluoro-1-oxospiro[3H-isoquinoline-4,1′-(spiro[2.2]pentan-1-yl)]-2-yl)acetate: To a solution of 6-bromospiro[2,3-dihydroisoquinoline-4,1′-(spiro[2.2]pentan-1-yl)]-1-one (150 mg, 0.54 mmol) in DMF (2 mL) at 0° C. was added NaH (26 mg, 0.65 mmol, 60% purity). The mixture was stirred at 0° C. for 0.5 h and then methyl 2-bromoacetate (83 mg, 0.54 mmol) was added. The mixture was stirred at 20° C. for 2 h. The reaction mixture was cooled to 0° C., diluted with sat. aq. NH₄Cl (10 mL), and extracted with DCM (4×5 mL). The combined organic layers were washed with brine (10 mL), dried over anhydrous Na₂SO₄, filtered, and concentrated under reduced pressure. The residue was purified by silica gel column chromatography. LCMS: m/z=350.0, 352.0 [M+H]⁺.

N-(5-fluoropyrimidin-2-yl)-2-(6-bromo-1-oxospiro[3H-isoquinoline-4,1′-(spiro[2.2]pentan-1-yl)]-2-yl)acetamide: To a mixture of methyl 2-(6-bromo-5-fluoro-1-oxospiro[3H-isoquinoline-4,1′-(spiro[2.2]pentan-1-yl)]-2-yl)acetate (80 mg, 0.23 mmol), 5-fluoropyrimidin-2-amine (52 mg, 0.46 mmol) in THF (1.5 mL) and toluene (1.5 mL) was added AlMe₃ (2 M in toluene, 0.3 mL). The mixture was stirred for 3 h at 90° C. The reaction mixture was cooled to 0° C., poured into water (10 mL), and extracted with EtOAc (4×5 mL). The combined organic layers were washed with brine (2×10 mL), dried over anhydrous Na₂SO₄, filtered, and concentrated under reduced pressure. The residue was purified by reverse-phase preparative HPLC. LCMS: m/z=431.0, 432.9, [M+H]⁺. ¹H NMR (400 MHz, CDCl₃): δ 9.00 (br s, 1H), 8.48 (s, 2H), 8.01 (d, J=8.4 Hz, 1H), 7.45 (dd, J=2.0, 8.4 Hz, 1H), 7.25 (d, J=2.0 Hz, 1H), 4.71-4.41 (m, 2H), 3.72-3.56 (m, 2H), 1.51-1.41 (m, 2H), 1.14-1.06 (m, 1H), 1.01-0.86 (m, 3H).

### Example 82

### 2-[6-(fluoromethoxy)-1-oxospiro[3H-isoquinoline-4,1′-cyclopropane]-2-yl]-N-pyrimidin-2-ylacetamide (82)

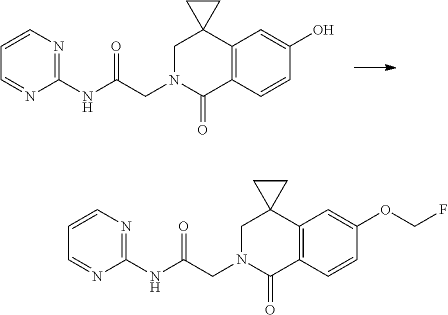 [SMILES: O=C(CN1CC2(CC2)c2cc(O)ccc2C1=O)Nc1ncccn1 ; O=C(CN1CC2(CC2)c2cc(OCF)ccc2C1=O)Nc1ncccn1]

To a solution of 2-[6-hydroxy-1-oxospiro[3H-isoquinoline-4,1′-cyclopropane]-2-yl]-N-pyrimidin-2-ylacetamide (50 mg, 0.15 mmol) in DMF (1 mL) was added Cs₂CO₃ (101 mg, 0.30 mmol) followed by fluoromethyl 4-methylbenzenesulfonate (47 mg, 0.23 mmol) as a solution in DMF (1 mL). The mixture was stirred at 20° C. for 16 h. The mixture was poured into sat. aq. NaHCO₃ (5 mL) and extracted with EtOAc (3×3 mL). The combined organic layers were washed with brine (5 mL), dried over anhydrous Na₂SO₄, filtered, and concentrated under reduced pressure. The residue was purified by reverse-phase HPLC. LCMS: m/z: 357.1 [M+H]⁺. ¹H NMR (400 MHz, CDCl₃): δ 8.84 (br s, 1H), 8.61 (d, J=5.2 Hz, 2H), 8.17 (d, J=8.8 Hz, 1H), 7.06-6.99 (m, 2H), 6.53 (s, 1H), 5.74 (d, J=54.4, 2H), 4.64 (br s, 2H), 3.54 (s, 2H), 1.12 (br s, 2H), 1.07 (br s, 2H).

### Example 83

### 2-(6-bromo-5-chloro-1-oxospiro[3H-isoquinoline-4,1′-cyclopropane]-2-yl)-N-(5-fluoropyrimidin-2-yl)acetamide (83)

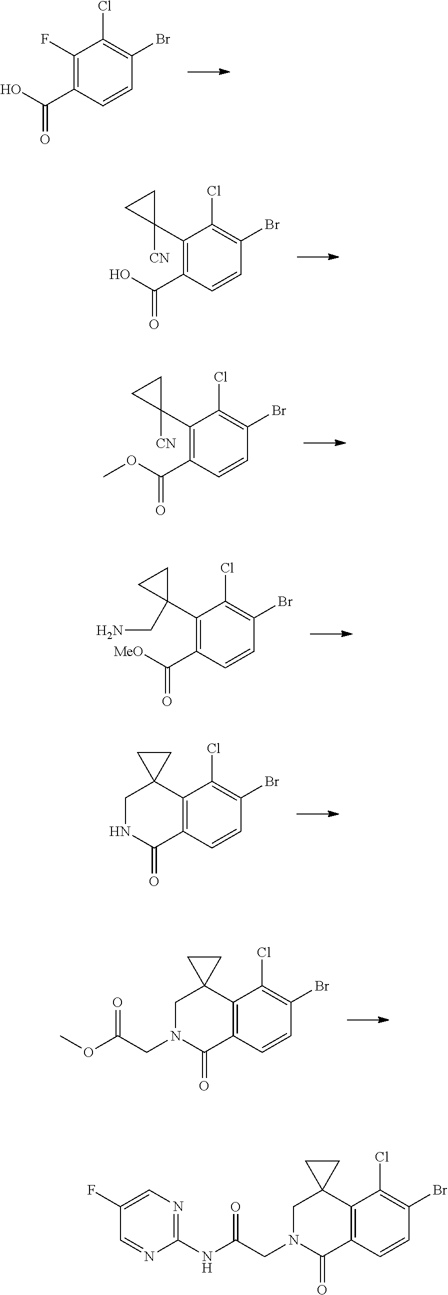

4-bromo-3-chloro-2-(1-cyanocyclopropyl)benzoic acid: To a solution of 4-bromo-3-chloro-2-fluorobenzoic acid (2.0 g, 7.89 mmol), cyclopropanecarbonitrile (1.06 g, 15.8 mmol) in THF (30 mL) was added KHMDS (1 M in THF, 21 mL) at −40° C. The mixture was stirred at 40° C. for 3 h. The reaction mixture was cooled to 0° C., diluted with water (20 mL) and cone. HCl (1.3 mL), adjusted to pH=8 with sat. aq. NaHCO₃, and extracted with MTBE (3×10 mL). The organics were discarded. The aqueous phase was adjusted back to pH=3 with aq. HCl (3 M) and extracted with EtOAc (4×10 mL). The combined organic layers were washed with brine (2×10 mL), dried over anhydrous Na₂SO₄, filtered, and concentrated under reduced pressure. LCMS: m/z=297.9, 300.0 [M−H]⁻.

Methyl 4-bromo-3-chloro-2-(1-cyanocyclopropyl)benzoate: To a solution of 4-bromo-3-chloro-2-(1-cyanocyclopropyl)benzoic acid (1.95 g, 6.49 mmol) and K₂CO₃ (1.35 g, 9.73 mmol) in DMF (20 mL) was added CH₃I (1.01 g, 7.14 mmol, 0.44 mL). The mixture was stirred at 20° C. for 3 h. The reaction mixture was cooled to 0° C., diluted with water (40 mL), and extracted with EtOAc (3×15 mL). The combined organic layers were washed with brine (3×10 mL), dried over anhydrous Na₂SO₄, filtered, and concentrated under reduced pressure. The residue was purified by silica gel column chromatography. LCMS: m/z=313.9, 315.9 [M+H]⁺.

Methyl 2-(1-(aminomethyl)cyclopropyl)-4-bromo-3-chlorobenzoate: To a mixture of methyl 4-bromo-3-chloro-2-(1-cyanocyclopropyl)benzoate (1.6 g, 5.09 mmol) and dichlorocobalt (1.32 g, 10.2 mmol) in MeOH (20 mL) and THF (5 mL) at 0° C. was added NaBH₄ (962 mg, 25.4 mmol). The mixture was stirred at 20° C. for 3 h. The reaction mixture was cooled to 0° C., diluted with sat. aq. NH₄Cl (40 mL), and extracted with DCM (4×15 mL). The combined organic layers were washed with brine (20 mL), dried over anhydrous Na₂SO₄, filtered, and concentrated under reduced pressure to provide a residue that was used directly. LCMS: m/z=317.8, 319.9 [M+H]⁺.

6-bromo-5-chlorospiro[2,3-dihydroisoquinoline-4,1′-cyclopropane]-1-one: To a solution of methyl 2-(1-(aminomethyl)cyclopropyl)-4-bromo-3-chlorobenzoate (1.6 g, 5.02 mmol) in 1,4-dioxane (30 mL) was added DIPEA (1.30 g, 10.04 mmol). The mixture was stirred at 90° C. for 3 h. The reaction mixture was cooled to 0° C., diluted with water (30 mL), and extracted with EtOAc (3×15 mL). The combined organic layers were washed with aq. HCl (0.5 M, 2×10 mL) and brine (10 mL), dried over anhydrous Na₂SO₄, filtered, and concentrated under reduced pressure to provide a residue that was used directly. LCMS: m/z=285.8, 287.7 [M+H]⁺.

Methyl 2-(6-bromo-5-chloro-1-oxo-spiro[3H-isoquinoline-4,1′-cyclopropane]-2-yl)acetate: To a solution of 6-bromo-5-chlorospiro[2,3-dihydroisoquinoline-4,1′-cyclopropane]-1-one (600 mg, 2.09 mmol) in DMF (15 mL) at 0° C. was added NaH (101 mg, 2.51 mmol, 60% purity). The mixture was stirred at 0° C. for 0.5 h and then methyl 2-bromoacetate (320 mg, 2.09 mmol, 0.2 mL) was added. The reaction mixture was stirred at 20° C. for 2 h. The reaction mixture was cooled to 0° C., diluted with sat. aq. NH₄Cl (30 mL), and extracted with EtOAc (4×10 mL). The combined organic layers were washed with brine (3×10 mL), dried over anhydrous Na₂SO₄, filtered, and concentrated under reduced pressure. The residue was purified by silica gel column chromatography. LCMS: m/z=357.8, 359.8 [M+H]⁺.

2-(6-bromo-5-chloro-1-oxospiro[3H-isoquinoline-4,1′-cyclopropane]-2-yl)-N-(5-fluoropyrimidin-2-yl)acetamide: To a mixture of 5-fluoropyrimidin-2-amine (126 mg, 1.12 mmol), methyl 2-(6-bromo-5-chloro-1-oxo-spiro[3H-isoquinoline-4,1′-cyclopropane]-2-yl)acetate (200 mg, 0.56 mmol) in toluene (3 mL) and THF (3 mL) was added AlMe₃ (2 M in toluene, 0.8 mL). The mixture was stirred for 3 h at 90° C. The reaction mixture was cooled to 0° C., diluted with H₂O (10 mL), and extracted with EtOAc (4×5 mL). The combined organic layers were washed with brine (2×10 mL), dried over anhydrous Na₂SO₄, filtered, and concentrated under reduced pressure. The residue was purified by reverse-phase preparative HPLC. LCMS: m/z=439.0, 441.0 [M+H]⁺. ¹H NMR (400 MHz, DMSO-d₆): δ 10.97 (br s, 1H), 8.76 (s, 2H), 7.87-7.75 (m, 2H), 4.50 (s, 2H), 3.41 (s, 2H), 1.83-1.79 (m, 2H), 1.16-1.12 (m, 2H).

### Example 84

### 2-[7-fluoro-1-oxo-6-(trifluoromethyl)spiro[3H-isoquinoline-4,1′-cyclopropane]-2-yl]-N-(5-fluoropyrimidin-2-yl)acetamide (84)

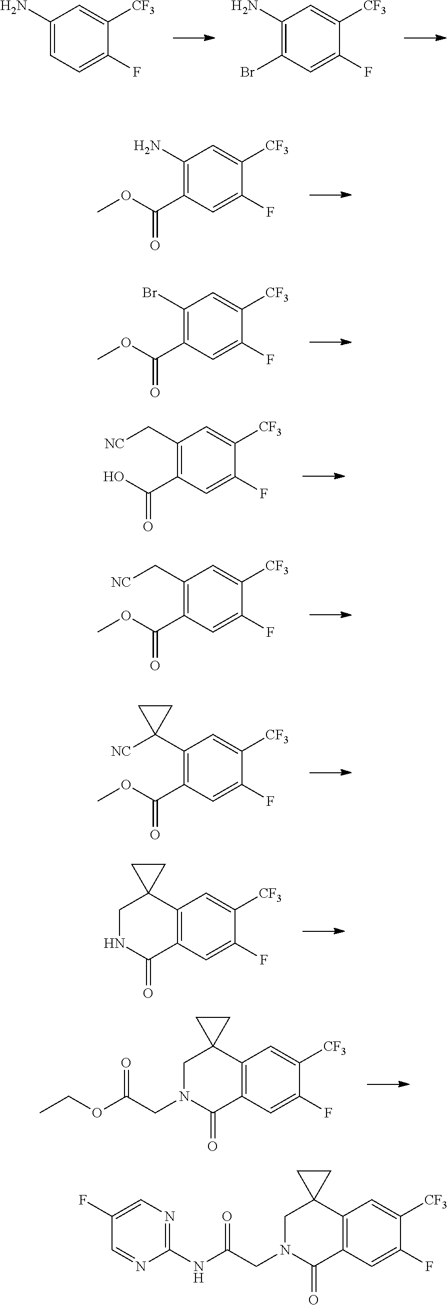

2-bromo-4-fluoro-5-(trifluoromethyl)aniline: To a solution of 4-fluoro-3-(trifluoromethyl)aniline (25 g, 139.6 mmol) in DCM (80 mL) was added NBS (24.8 g, 139.6 mmol) in DCM (160 mL). The mixture was stirred at 20° C. for 16 h. The reaction mixture was diluted with sat. aq. NaHCO₃ (200 mL) and extracted with DCM (3×100 mL). The combined organic layers were washed with brine (100 mL), dried over anhydrous Na₂SO₄, filtered, and concentrated under reduced pressure. The residue was purified by silica gel column chromatography.

Methyl 2-amino-5-fluoro-4-(trifluoromethyl)benzoate: To a solution of 2-bromo-4-fluoro-5-(trifluoromethyl)aniline (14 g, 54.3 mmol), Et₃N (16.5 g, 162.8 mmol) in DMSO (150 mL) and MeOH (120 mL) were added Pd(OAc)₂ (1.22 g, 5.43 mmol) and DPPF (6.02 g, 10.9 mmol). The suspension was degassed under vacuum and purged with CO several three times. The mixture was stirred under CO (50 psi) at 80° C. for 16 h. The mixture was filtered and concentrated under reduced pressure. The residue was purified by silica gel column chromatography. ¹H NMR (400 MHz, CDCl₃): δ 7.65 (d, J=12.0 Hz, 1H), 6.88 (d, J=4 Hz, 1H), 5.76 (br s, 2H), 3.90 (s, 3H).

Methyl 2-bromo-5-fluoro-4-(trifluoromethyl)benzoate: To a solution of CuBr (3.63 g, 25.3 mmol) and CuBr₂ (5.65 g, 25.3 mmol) in MeCN (40 mL) were added t-BuONO (6.52 g, 63.25 mmol, 7.52 mL) and methyl 2-amino-5-fluoro-4-(trifluoromethyl)benzoate (10 g, 42.2 mmol). The mixture was stirred at 65° C. for 2 h. The mixture was diluted with ice-cold water (50 mL) and extracted with EtOAc (3×40 mL). The combined organic layers were washed with brine (20 mL), dried over anhydrous Na₂SO₄, filtered, and concentrated under reduced pressure. The residue was purified by silica gel column chromatography. ¹H NMR (400 MHz, CDCl₃): δ 7.90 (d, J=6.4 Hz, 1H), 7.64 (d, J=10.0 Hz, 1H), 3.98 (s, 3H).

2-(cyanomethyl)-5-fluoro-4-(trifluoromethyl)benzoic acid: A mixture of methyl 2-bromo-5-fluoro-4-(trifluoromethyl)benzoate (9.2 g, 30.6 mmol) and ditert-butyl(cyclopentyl)phosphane;dichloropalladium;iron (1.99 g, 3.1 mmol), 4-(4,4,5,5-tetramethyl-1,3,2-dioxaborolan-2-yl)isoxazole (11.9 g, 61.1 mmol) and Cs₂CO₃ (29.9 g, 91.68 mmol) in THF (200 mL) and H₂O (20 mL) was stirred at 110° C. for 16 h. The reaction mixture was concentrated to remove the organics and diluted with sat. aq. Na₂CO₃ (50 mL). The mixture was washed with MTBE (3×100 mL) and the organics were discarded. The aqueous layer was acidified to pH=3 and extracted with DCM (3×100 mL). The combined organic layers were washed with brine (50 mL), dried over anhydrous Na₂SO₄, filtered, and concentrated to provide residue that was used directly. LCMS: m/z=246.0 [M−H]⁻.

Methyl 2-(cyanomethyl)-5-fluoro-4-(trifluoromethyl)benzoate: To a solution of 2-(cyanomethyl)-5-fluoro-4-(trifluoromethyl)benzoic acid (9.0 g, 36.4 mmol) in THF (100 mL) was added diazomethyl(trimethyl)silane (2 M in n-hexane, 27.3 mL). The mixture was stirred at 20° C. for 1 h. The mixture was diluted with ice-cold water (100 mL) and extracted with EtOAc (3×50 mL). The combined organic layers were washed with brine (50 mL), dried over anhydrous Na₂SO₄, filtered, and concentrated under reduced pressure. The residue was purified by silica gel column chromatography.

Methyl 2-(1-cyanocyclopropyl)-5-fluoro-4-(trifluoromethyl)benzoate: To a solution of NaH (704 mg, 17.6 mmol, 60% purity) in DMSO (20 mL) were added 1-bromo-2-chloro-ethane (1.32 g, 9.2 mmol) and methyl 2-(cyanomethyl)-5-fluoro-4-(trifluoromethyl)benzoate (2.0 g, 7.7 mmol). The mixture was stirred at 20° C. for 2 h. The mixture was quenched with sat. aq. NH₄Cl (30 mL) and extracted with EtOAc (3×15 mL). The combined organic layers were washed with brine (15 mL), dried over anhydrous Na₂SO₄, filtered, and concentrated under reduced pressure. The residue was purified by silica gel column chromatography. ¹H NMR (400 MHz, CDCl₃): δ 7.80 (d, J=10.0 Hz, 1H), 7.72 (d, J=6.4 Hz, 1H), 4.05 (s, 3H), 1.84-1.79 (m, 2H), 1.36-1.30 (m, 2H).

7-fluoro-6-trifluorospiro[2,3-dihydroisoquinoline-4,1′-cyclopropane]-1-one: To a solution of methyl 2-(1-cyanocyclopropyl)-5-fluoro-4-(trifluoromethyl)benzoate (800 mg, 2.79 mmol) in MeOH (10 mL) at 0° C. were added NaBH₄ (527 mg, 13.9 mmol) and dichlorocobalt (1.08 g, 8.36 mmol). The mixture was stirred at 20° C. for 3 h. The mixture was quenched with sat. aq. NH₄Cl (200 mL) and extracted with EtOAc (3×10 mL). The combined organic layers were washed with brine (10 mL), dried over anhydrous Na₂SO₄, filtered, and concentrated under reduced pressure. The crude residue was triturated with MTBE (5 mL) at 20° C. for 30 min and filtered to provide a solid that was used directly. LCMS: m/z=260.2 [M+H]⁺.

Ethyl 2-(7-fluoro-1-oxo-6-(trifluoromethyl)spiro[3H-isoquinoline-4,1′-cyclopropane]-2-yl)acetate: To a solution of 7-fluoro-6-trifluorospiro[2,3-dihydroisoquinoline-4,1′-cyclopropane]-1-one (450 mg, 1.74 mmol) in DMF (5 mL) at 0° C. were added Cs₂CO₃ (1.13 g, 3.47 mmol) and ethyl 2-iodoacetate (483 mg, 2.26 mmol). The mixture was stirred at 20° C. for 2 h. The mixture was quenched with sat. aq. NH₄Cl (10 mL) and extracted with EtOAc (3×5 mL). The combined organic layers were washed with brine (5 mL), dried over anhydrous Na₂SO₄, filtered, and concentrated under reduced pressure. The residue was purified by silica gel column chromatography.

2-[7-fluoro-1-oxo-6-(trifluoromethyl)spiro[3H-isoquinoline-4,1′-cyclopropane]-2-yl]-N-(5-fluoropyrimidin-2-yl)acetamide: To a solution of ethyl 2-(7-fluoro-1-oxo-6-(trifluoromethyl)spiro[3H-isoquinoline-4,1′-cyclopropane]-2-yl)acetate (100 mg, 0.29 mmol) in toluene (2 mL) was added trimethyl-(4-trimethylalumanuidyl-1,4-diazoniabicyclo[2.2.2]octan-1-yl)alumanuide (74 mg, 0.29 mmol) and 5-fluoropyrimidin-2-amine (39 mg, 0.35 mmol). The mixture was stirred at 60° C. for 7 h. The mixture was quenched with sat. aq. NH₄Cl (5 mL) and extracted with EtOAc (3×2 mL). The combined organic layers were washed with brine (5 mL), dried over anhydrous Na₂SO₄, filtered, and concentrated under reduced pressure. The residue was purified by reverse-phase HPLC. LCMS: m/z=413.0 [M+H]⁺. ¹H NMR (400 MHz, CDCl₃): 11.09 (s, 1H), 8.77 (s, 2H), 7.84 (d, J=8.0 Hz, 1H), 7.40 (d, J=6.4 Hz, 1H), 4.54 (s, 2H), 3.54 (s, 2H), 1.27-1.21 (m, 2H), 1.11-1.06 (m, 2H).

### Example 85

### N-(5-cyano-3-fluoropyridin-2-yl)-2-[1-oxo-6-(trifluoromethyl)spiro[3H-isoquinoline-4,1′-cyclopropane]-2-yl]acetamide (85)

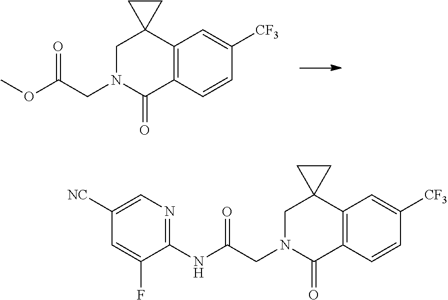 [SMILES: COC(=O)CN1CC2(CC2)c2cc(C(F)(F)F)ccc2C1=O ; N#Cc1cnc(NC(=O)CN2CC3(CC3)c3cc(C(F)(F)F)ccc3C2=O)c(F)c1]

To a solution of methyl 2-(1-oxo-6-trifluoromethylspiro[3H-isoquinoline-4,1′-cyclopropane]-2-yl)acetate (60 mg, 0.19 mmol) and 6-amino-5-fluoronicotinonitrile (53 mg, 0.3 mmol) in toluene (1 mL) was added DABAL-Me₃ (49 mg, 0.19 mmol). The mixture was stirred at 110° C. for 2 h. The reaction mixture was quenched by addition of water (5 mL) and extracted with EtOAc (3×5 mL). The combined organic phase was dried over anhydrous Na₂SO₄, filtered, and concentrated under reduced pressure. The residue was purified by reverse-phase preparative HPLC. LCMS: m/z=419.1 [M+H]⁺. H NMR (400 MHz, CDCl₃): δ 9.03 (br s, 1H), 8.51 (d, J=1.6 Hz, 1H), 8.26 (d, J=8.0 Hz, 1H), 7.70-7.67 (m, 1H), 7.59 (d, J=8.0 Hz, 1H), 7.11 (s, 1H), 4.62 (s, 2H), 3.60 (s, 2H), 1.22-1.18 (m, 2H), 1.16-1.11 (m, 2H).

### Example 86

### 2-[(2′s,4r)-6-bromo-1-oxo-2′-(trifluoromethyl)spiro[3H-isoquinoline-4,1′-cyclopropane]-2-yl]-N-(5-fluoropyrimidin-2-yl)acetamide (86)

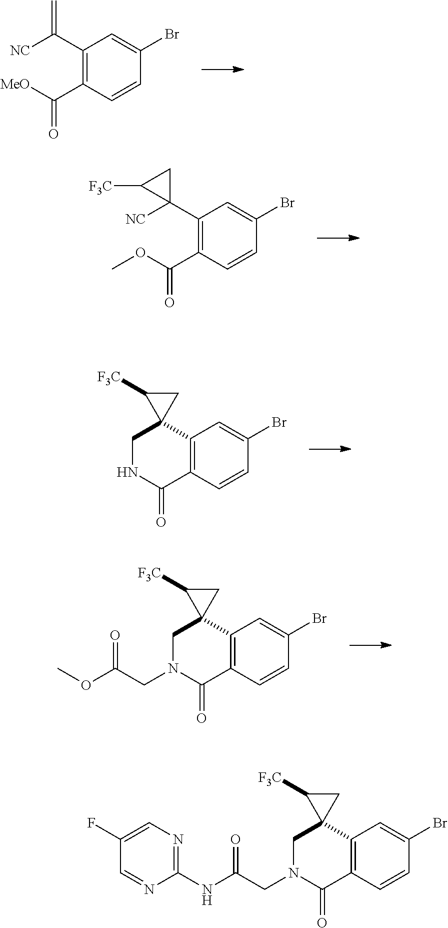

Methyl 4-bromo-2-(1-cyano-2-(trifluoromethyl)cyclopropyl)benzoate: To a mixture of methyl 4-bromo-2-(1-cyanovinyl)benzoate (460 mg, 1.73 mmol), diphenyl(2,2,2-trifluoroethyl)sulfonium trifluoromethanesulfonate (1.81 g, 4.32 mmol), and CsF (657 mg, 4.32 mmol) in DMA (5 mL) was added (TPP)FeCl (61 mg, 0.086 mmol). The mixture was stirred at 60° C. for 12 h. The mixture was diluted with water (60 mL) and extracted with EtOAc (3×60 mL). The combined organic layers were washed with brine (60 mL), dried over anhydrous Na₂SO₄, filtered and concentrated under reduced pressure. The residue was purified by silica gel column chromatography. ¹H NMR (400 MHz, CDCl₃): δ 7.92 (d, J=8.4 Hz, 1H), 7.66-7.61 (m, 2H), 4.00 (s, 3H), 2.34-2.25 (m, 1H), 2.17-2.14 (m, 1H), 1.73-1.68 (m, 1H).

(2′s,4r)-6-bromo-2′-(trifluoromethyl)spiro[2,3-dihydroisoquinoline-4,1′-cyclopropane]-1-one: To a solution of methyl 4-bromo-2-(1-cyano-2-(trifluoromethyl)cyclopropyl)benzoate (220 mg, 0.63 mmol) in MeOH (2.0 mL) and water (0.2 mL) at 0° C. were added NaBH₄ (143 mg, 3.79 mmol) and dichlorocobalt (328 mg, 2.53 mmol). The mixture was stirred at 40° C. for 12 h. The reaction mixture was diluted with sat. aq. NH₄Cl (30 mL) and extracted with DCM (4×30 mL). The combined organic layers were washed with brine (30 mL), dried over anhydrous Na₂SO₄, filtered, and concentrated under reduced pressure. The residue was purified by silica gel column chromatography. LCMS: m/z=320.0, 321.9 [M+H]⁺.

Methyl 2-[(2′s,4r)-6-bromo-1-oxo-2′-(trifluoromethyl)spiro[3H-isoquinoline-4,1′-cyclopropane]-2-yl]acetate: To a solution of (2′s,4r)-6-bromo-2′-(trifluoromethyl)spiro[2,3-dihydroisoquinoline-4,1′-cyclopropane]-1-one (80 mg, 0.25 mmol) and methyl 2-bromoacetate (42 mg, 0.27 mmol) in DMF (1.0 mL) was added Cs₂CO₃ (122 mg, 0.37 mmol). The mixture was stirred at 40° C. for 2 h. The mixture was diluted with water (20 mL) and extracted with EtOAc (3×20 mL). The combined organic layers were washed with brine (20 mL), dried over anhydrous Na₂SO₄, filtered and concentrated under reduced pressure. The residue was purified by silica gel preparatory TLC. LCMS: m/z=392.0, 394.0 [M+H]⁺.

2-[(2′s,4r)-6-bromo-1-oxo-2′-(trifluoromethyl)spiro[3H-isoquinoline-4,1′-cyclopropane]-2-yl]-N-(5-fluoropyrimidin-2-yl)acetamide: To a solution of methyl 2-[(2′s,4r)-6-bromo-1-oxo-2′-(trifluoromethyl)spiro[3H-isoquinoline-4,1′-cyclopropane]-2-yl]acetate (35 mg, 0.089 mmol) and 5-fluoropyrimidin-2-amine (20 mg, 0.18 mmol) in toluene (1.0 mL) was added DABAL-Me₃ (23 mg, 0.089 mmol). The mixture was stirred at 60° C. for 5 h. The reaction mixture was quenched by addition of water(10 mL) and extracted with EtOAc (3×10 mL). The combined organic layers were washed with brine (10 mL), dried over anhydrous Na₂SO₄, filtered and concentrated under reduced pressure. The residue was purified by reverse-phase HPLC. LCMS: m/z=473.0, 475.0 [M+H]⁺. ¹H NMR (400 MHz, CDCl₃): δ 8.79 (br s, 1H), 8.49 (s, 2H), 8.07 (d, J=8.4 Hz, 1H), 7.57-7.55 (m, 1H), 7.03 (d, J=1.6 Hz, 1H), 5.12-5.08 (m, 1H), 4.26 (d, J=16.8 Hz, 1H), 4.07 (d, J=13.6 Hz, 1H), 3.64 (d, J=13.2 Hz, 1H), 2.06-1.95 (m, 1H), 1.71-1.67 (m, 1H), 1.60-1.57 (m, 1H).

### Example 87

### 2-(6-cyclobutyl-1-oxospiro[3H-isoquinoline-4,1′-cyclopropane]-2-yl)-N-(5-fluoropyrimidin-2-yl)acetamide (87)

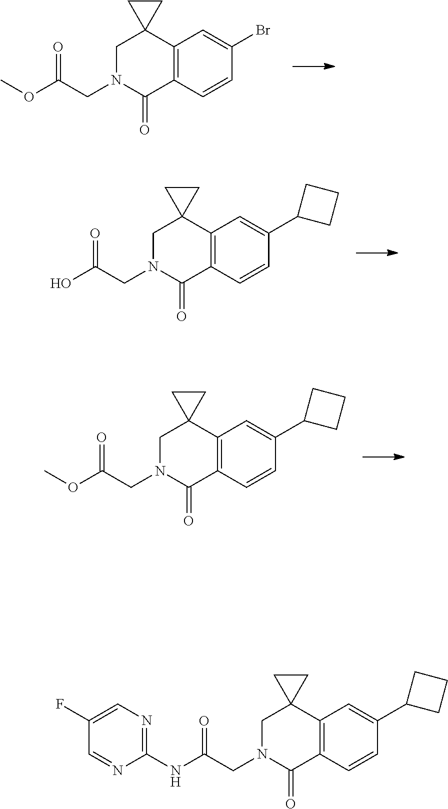 [SMILES: COC(=O)CN1CC2(CC2)c2cc(Br)ccc2C1=O ; O=C(O)CN1CC2(CC2)c2cc(C3CCC3)ccc2C1=O ; COC(=O)CN1CC2(CC2)c2cc(C3CCC3)ccc2C1=O ; O=C(CN1CC2(CC2)c2cc(C3CCC3)ccc2C1=O)Nc1ncc(F)cn1]

2-(6-cyclobutyl-1-oxo-1H-spiro[cyclopropane-1,4′-isoquinolin]-2(3H)-yl)acetic acid: To a mixture of methyl 2-(6-bromo-1-oxo-spiro[3H-isoquinoline-4,1′-cyclopropane]-2-yl)acetate (300 mg, 0.92 mmol) in THF (2.0 mL) was added Pd(dppf)Cl₂ (68 mg, 0.092 mmol). The mixture was sparged with N₂ for 5 min. Cyclobutylzinc(II) bromide (0.5 M in THF, 18.5 mL) was added to the mixture. The reaction mixture was stirred at 80° C. for 12 h. The mixture was quenched by addition of water (20 mL), the pH was adjusted to 2-3 with aq. HCl (2 M) and extracted with EtOAc (3×20 mL). The combined organic layers were washed with brine (30 mL), dried over anhydrous Na₂SO₄, filtered and concentrated under reduced pressure to provide a residue that was used directly. LCMS: m/z=286.1 [M+H]⁺.

Methyl 2-(6-bromo-1-oxo-spiro[3H-isoquinoline-4,1′-cyclopropane]-2-yl)acetate: To a solution of 2-(6-cyclobutyl-1-oxo-1H-spiro[cyclopropane-1,4′-isoquinolin]-2(3H)-yl)acetic acid (60 mg, 0.21 mmol) and K₂CO₃ (58 mg, 0.42 mmol) in DMF (1.2 mL) at 0° C. was added CH₃I (45 mg, 0.31 mmol) dropwise. The mixture was stirred at 20° C. for 2 h. The reaction mixture was quenched by addition of water (10 mL) and extracted with EtOAc (3×10 mL). The combined organic layers were washed with brine (10 mL), dried over anhydrous Na₂SO₄, filtered and concentrated under reduced pressure. The residue was purified by silica gel preparatory TLC. LCMS: m/z=300.1 [M+H]⁺.

2-(6-cyclobutyl-1-oxo-spiro[3H-isoquinoline-4,1′-cyclopropane]-2-yl)-N-(5-fluoropyrimidin-2-yl)acetamide: To a solution of methyl 2-(6-bromo-1-oxo-spiro[3H-isoquinoline-4,1′-cyclopropane]-2-yl)acetate (60 mg, 0.20 mmol) and 5-fluoropyrimidin-2-amine (45 mg, 0.40 mmol) in DCE (0.6 mL) at 0° C. was added AlMe₃ (2 M in toluene, 0.10 mL). The mixture was stirred at 60° C. for 2 h. The reaction was quenched by addition of MeCN:H₂O (4:1 mixture, 10 mL) and filtered. The filtrate was extracted with EtOAc (3×10 mL). The combined organic layers were washed with brine (10 mL), dried over with anhydrous Na₂SO₄, filtered and concentrated under reduced pressure. The residue was purified by reverse-phase preparative HPLC. LCMS: m/z=381.2 [M+H]⁺. ¹H NMR (400 MHz, CDCl₃): δ 9.20 (s, 1H), 8.49 (s, 2H), 8.09 (d, J=8.0 Hz, 1H), 7.20 (d, J=8.0 Hz, 1H), 6.66 (s, 1H), 4.54 (s, 2H). 3.58-3.53 (m, 3H), 2.40-2.33 (m, 2H), 2.19-2.03 (m, 3H), 1.91-1.84 (m, 1H), 1.16-1.13 (m, 2H), 1.04-1.01 (m, 2H).

### Example 88

### 2-[6-(2,2-difluorocyclopropyl)-1-oxo-spiro[3H-isoquinoline-4,1′-cyclopropane]-2-yl]-N-(5-fluoropyrimidin-2-yl)acetamide (88)

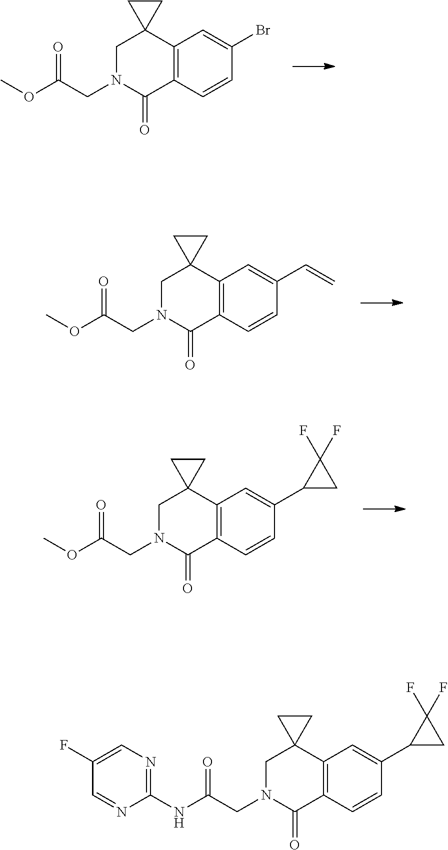

Methyl 2-(6-vinyl-1-oxo-spiro[3H-isoquinoline-4,1′-cyclopropane]-2-yl)acetate: To a solution of methyl 2-(6-bromo-1-oxo-spiro[3H-isoquinoline-4,1′-cyclopropane]-2-yl)acetate (1.5 g, 4.6 mmol), potassium trifluoro(vinyl)borate (3.10 g, 23.1 mmol) in 1,4-dioxane (35 mL) were added Pd(dppf)Cl₂ (339 mg, 0.46 mmol) and CsF (2.11 g, 13.9 mmol). The mixture was stirred at 90° C. for 4 h. The reaction mixture was poured into water (30 mL) and extracted with EtOAc (4×15 mL). The combined organic layers were washed with brine (2×20 mL), dried over anhydrous Na₂SO₄, filtered and concentrated under reduced pressure. The residue was purified by silica gel column chromatography. LCMS: m/z=272.1 [M+H]⁺.

Methyl 2-(6-(2,2-difluorocyclopropyl)-1-oxo-spiro[3H-isoquinoline-4,1′-cyclopropane]-2-yl)acetate: To a solution of methyl 2-(6-vinyl-1-oxo-spiro[3H-isoquinoline-4,1′-cyclopropane]-2-yl)acetate (100 mg, 0.37 mmol) in CH₃CN (1.9 mL) were added TMSCF₃ (262 mg, 1.84 mmol, 0.3 mL) and NaI (6 mg, 0.04 mmol). The mixture was stirred at 110° C. for 2 h. The reaction mixture was poured into water (10 mL) and extracted with MTBE (3×5 mL). The combined organic layers were washed with brine (5 mL), dried over anhydrous Na₂SO₄, filtered, and concentrated under reduced pressure. The residue was purified by silica gel column chromatography. LCMS: m/z=322.1 [M+H]⁺.

2-[6-(2,2-difluorocyclopropyl)-1-oxospiro[3H-isoquinoline-4,1′-cyclopropane]-2-yl]-N-(5-fluoropyrimidin-2-yl)acetamide: To a mixture of methyl 2-(6-(2,2-difluorocyclopropyl)-1-oxo-spiro[3H-isoquinoline-4,1′-cyclopropane]-2-yl)acetate (90 mg, 0.28 mmol), 5-fluoropyrimidin-2-amine (63 mg, 0.56 mmol) in THF (1.0 mL) and toluene (1.0 mL) was added AlMe₃ (2 M in toluene, 0.42 mL). The mixture was stirred for 3 h at 90° C. The reaction mixture was cooled to 0° C., diluted with water (10 mL), and extracted with EtOAc (4×5 mL). The combined organic layers were washed with brine (2×10 mL), dried over anhydrous Na₂SO₄, filtered, and concentrated under reduced pressure. The residue was purified by reverse-phase preparative HPLC. LCMS: m/z=403.1 [M+H]⁺. ¹H NMR (400 MHz, CDCl₃): δ 9.11 (br s, 1H), 8.48 (s, 2H), 8.13 (d, J=8.0 Hz, 1H), 7.17 (d, J=8.0 Hz, 1H), 6.71 (s, 1H). 4.55 (br s, 2H), 3.54 (s, 2H), 2.80-2.71 (m, 1H), 1.94-1.82 (m, 1H), 1.71-1.62 (m, 1H), 1.19-1.00 (m, 4H).

### Example 89

### 2-[5-cyano-1-oxo-6-(trifluoromethyl)spiro[3H-isoquinoline-4,1′-cyclopropane]-2-yl]-N-(5-fluoropyrimidin-2-yl)acetamide (89)

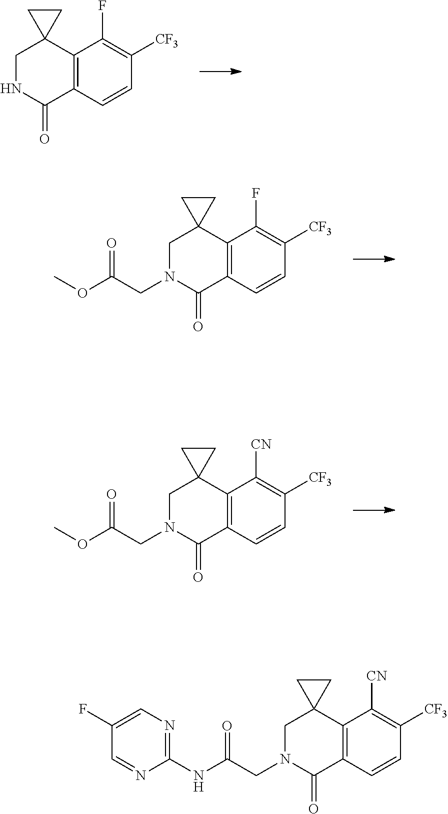 [SMILES: O=C1NCC2(CC2)c2c1ccc(C(F)(F)F)c2F ; COC(=O)CN1CC2(CC2)c2c(ccc(C(F)(F)F)c2F)C1=O ; COC(=O)CN1CC2(CC2)c2c(ccc(C(F)(F)F)c2C#N)C1=O ; N#Cc1c(C(F)(F)F)ccc2c1C1(CC1)CN(CC(=O)Nc1ncc(F)cn1)C2=O]

Methyl 2-(5-fluoro-1-oxo-6-(trifluoromethyl)spiro[3H-isoquinoline-4,1′-cyclopropane]-2-yl)acetate: To a solution of 5-fluoro-6-(trifluoromethyl)spiro[2,3-dihydroisoquinoline-4,1′-cyclopropane]-1-one (900 mg, 3.47 mmol) in DMF (10 mL) at 0° C. was added NaH (139 mg, 3.47 mmol, 60% purity in mineral oil). The mixture was stirred at 0° C. for 30 min followed by the addition of methyl 2-bromoacetate (797 mg, 5.21 mmol). The reaction mixture was stirred at 20° C. for 1.5 h. The reaction mixture was quenched by the addition of water (5 mL) and extracted with EtOAc (3×5 mL). The combined organics were dried over anhydrous Na₂SO₄, filtered, and concentrated under reduced pressure. The residue was purified by silica gel column chromatography. ¹H NMR (400 MHz, CDCl₃): δ 8.04-8.01 (m, 1H), 7.56-7.47 (m, 1H), 4.32 (s, 2H), 3.76 (s, 3H), 3.39 (s, 2H), 1.68-1.62 (m, 2H), 1.08-1.01 (m, 2H).

Methyl 2-(5-cyano-1-oxo-6-(trifluoromethyl)spiro[3H-isoquinoline-4,1′-cyclopropane]-2-yl)acetate: To a solution of methyl 2-(5-fluoro-1-oxo-6-(trifluoromethyl)spiro[3H-isoquinoline-4,1′-cyclopropane]-2-yl)acetate (500 mg, 1.51 mmol) in DMF (10 mL) was added KCN (147 mg, 2.26 mmol). The mixture was stirred at 110° C. for 16 h. The reaction mixture was quenched by the addition of water (10 mL) and extracted with EtOAc (3×15 mL). The combined organics were concentrated under reduced pressure to provide a residue that was dissolved in DMF (50 mL). To this solution was added K₂CO₃ (294 mg, 2.13 mmol) and Mel (203 mg, 1.43 mmol). The reaction mixture was stirred at 20° C. for 3 h. The reaction mixture was diluted with water (20 mL) and extracted with EtOAc (3×15 mL). The combined organics were washed with brine (20 mL), dried over anhydrous Na₂SO₄, filtered, and concentrated under reduced pressure. The residue was purified by preparatory TLC. ¹H NMR (400 MHz, CDCl₃): δ 8.46 (d, J=8.2 Hz, 1H), 7.76 (d, J=8.2 Hz, 1H), 4.34 (s, 2H), 3.77 (s, 3H), 3.42 (s, 2H), 2.06-2.01 (m, 2H), 1.27-1.21 (m, 2H).

2-[5-cyano-1-oxo-6-(trifluoromethyl)spiro[3H-isoquinoline-4,1′-cyclopropane]-2-yl]-N-(5-fluoropyrimidin-2-yl)acetamide: To a solution of methyl 2-(5-cyano-1-oxo-6-(trifluoromethyl)spiro[3H-isoquinoline-4,1′-cyclopropane]-2-yl)acetate (100 mg, 0.29 mmol) and 5-fluoropyrimidin-2-amine (100 mg, 0.89 mmol) in THF (1.0 mL) and toluene (2.0 mL) was added AlMe₃ (0.44 mL, 2 M in toluene). The reaction mixture was stirred at 90° C. for 3 h. The reaction mixture was diluted with water (5 mL) and extracted with EtOAc (3×5 mL). The combined organics were dried over anhydrous Na₂SO₄, filtered, and concentrated under reduced pressure. The residue was purified by reverse-phase preparatory HPLC. LCMS: m/z=420.1 [M+H]⁺. ¹H NMR (400 MHz, CDCl₃): δ 8.70 (br s, 1H), 8.51-8.48 (m, 3H), 7.78 (br d, J=8.2 Hz, 1H), 4.75 (br s, 2H), 3.52 (br s, 2H), 2.06 (br s, 2H), 1.28 (br s, 2H).

### Example 90

### N-(5-fluoropyrimidin-2-yl)-2-(6-iodo-1-oxospiro[3H-isoquinoline-4,1′-cyclopropane]-2-yl)acetamide (90)

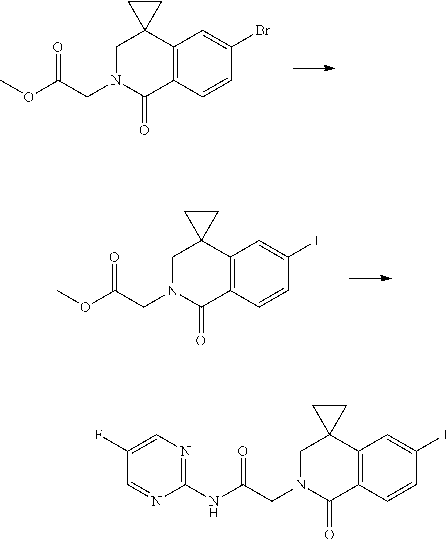 [SMILES: COC(=O)CN1CC2(CC2)c2cc(Br)ccc2C1=O ; COC(=O)CN1CC2(CC2)c2cc(I)ccc2C1=O ; O=C(CN1CC2(CC2)c2cc(I)ccc2C1=O)Nc1ncc(F)cn1]

Methyl 2-(6-iodo-1-oxo-spiro[3H-isoquinoline-4,1′-cyclopropane]-2-yl)acetate: To a solution of methyl 2-(6-bromo-1-oxo-spiro[3H-isoquinoline-4,1-cyclopropane]-2-yl)acetate (200 mg, 0.62 mmol) in toluene (4.0 mL) were added CuI (23 mg, 0.12 mmol), NaI (370 mg, 2.47 mmol), and cis-N,N′-dimethyl-1,2-diaminocyclohexane (35 mg, 0.25 mmol). The reaction mixture was degassed and purged with N₂ three times then stirred at 130° C. for 32 h. The mixture was diluted with water (5 mL) and extracted with EtOAc (3×5 mL). The combined organic layers were washed with brine (5 mL), dried over anhydrous Na₂SO₄, filtered, and concentrated under reduced pressure. The residue was purified by silica gel column chromatography. LCMS: m/z=372.0 [M+H]⁺. ¹H NMR (400 MHz, CDCl₃): δ 7.82 (d, J=8.2 Hz, 1H), 7.67 (dd, J=8.2, 1.3 Hz, 1H), 7.20 (d, J=1.2 Hz, 1H), 4.33 (s, 2H), 3.76 (s, 3H), 3.43 (s, 2H), 1.14-1.08 (m, 2H), 1.07-1.02 (m, 2H).

N-(5-fluoropyrimidin-2-yl)-2-(6-iodo-1-oxospiro[3H-isoquinoline-4,1′-cyclopropane]-2-yl)acetamide: To a solution of methyl 2-(6-iodo-1-oxo-spiro[3H-isoquinoline-4,1′-cyclopropane]-2-yl)acetate (200 mg, 0.54 mmol) and 5-fluoropyrimidin-2-amine (183 mg, 1.62 mmol) in toluene (2.0 mL) and THF (1.0 mL) was added AlMe₃ (0.89 mL, 2 M in toluene). The reaction mixture was stirred at 110° C. for 3 h. The reaction mixture was diluted with water (5 mL) and extracted with EtOAc (3×5 mL). The combined organics were dried over anhydrous Na₂SO₄, filtered, and concentrated under reduced pressure. The residue was purified by reverse-phase preparatory HPLC. LCMS: m/z=453.1 [M+H]⁺. ¹H NMR (400 MHz, CDCl₃): δ 8.97 (br s, 1H), 8.51-8.45 (s, 2H), 7.86 (d, J=8.2 Hz, 1H), 7.72-7.67 (m, 1H), 7.22 (d, J=1.6 Hz, 1H), 4.59-4.53 (br s, 2H), 3.53 (s, 2H), 1.16-1.11 (m, 2H), 1.10-1.04 (m, 2H).

### Example 91

### 2-(6-methoxy-1-oxospiro[3H-isoquinoline-4,1′-cyclopropane]-2-yl)-N-pyrimidin-2-ylacetamide (91)

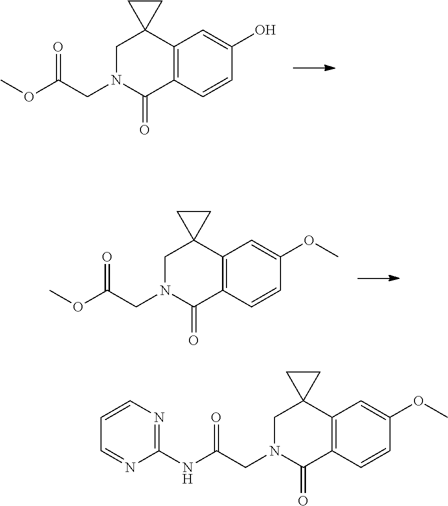

Methyl 2-(6-methoxy-1-oxo-spiro[3H-isoquinoline-4,1′-cyclopropane]-2-yl)acetate: To a solution of methyl 2-(6-hydroxy-1-oxo-spiro[3H-isoquinoline-4,1′-cyclopropane]-2-yl)acetate (100 mg, 0.38 mmol) in DMF (2.0 mL) at 0° C. were added K₂CO₃ (80 mg, 0.57 mmol) and Mel (54 mg, 0.38 mmol). The reaction mixture was stirred at 20° C. for 2 h. The reaction mixture was diluted with water (8 mL) and extracted with EtOAc (3×3 mL). The combined organics were washed with brine (5 mL), dried over anhydrous Na₂SO₄, filtered, and concentrated under reduced pressure. The residue was purified by silica gel chromatography. LCMS: m/z=276.1 [M+H]⁺.

2-(6-methoxy-1-oxospiro[3H-isoquinoline-4,1′-cyclopropane]-2-yl)-N-pyrimidin-2-ylacetamide: To a solution of methyl 2-(6-methoxy-1-oxo-spiro[3H-isoquinoline-4,1′-cyclopropane]-2-yl)acetate (60 mg, 0.22 mmol) in toluene (1.0 mL) and THF (1.0 mL) were added pyrimidin-2-amine (62 mg, 0.65 mmol) and AlMe₃ (0.33 mL, 2 M in toluene). The reaction mixture was stirred at 90° C. for 3 h. The reaction mixture was diluted with water (5 mL) and extracted with EtOAc (3×2 mL). The combined organics were washed with brine (5 mL), dried over anhydrous Na₂SO₄, filtered, and concentrated under reduced pressure. The residue was purified by reverse-phase preparatory HPLC. LCMS: m/z=339.1 [M+H]⁺. ¹H NMR (400 MHz, CDCl₃): δ 9.19 (br s, 1H), 8.62 (d, J=4.8 Hz, 2H), 8.13 (d, J=8.8 Hz, 1H), 7.02 (t, J=4.8 Hz, 1H), 6.83 (dd, J=2.8, 8.8 Hz, 1H), 6.34 (d, J=2.4 Hz, 1H), 4.61 (br s, 2H), 3.84 (s, 3H), 3.51 (s, 2H), 1.13-1.07 (m, 2H), 1.06-1.01 (m, 2H).

### Example 92

### 2-(6-bromo-1-oxospiro[3H-isoquinoline-4,3′-oxetane]-2-yl)-N-pyrimidin-2-ylacetamide (92)

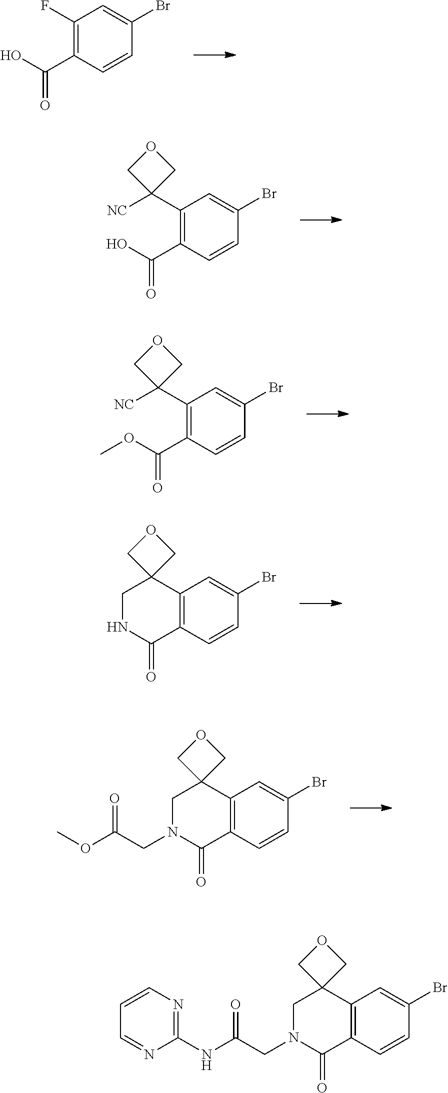

4-bromo-2-(3-cyanooxetan-3-yl)benzoic acid: To a solution of 4-bromo-2-fluorobenzoic acid (500 mg, 2.28 mmol) and oxetane-3-carbonitrile (500 mg, 6.02 mmol) in THF (10 mL) at −78° C. was added NaHMDS (5.02 mL, 1 M in THF). The reaction mixture was stirred at 25° C. for 12 h. The reaction mixture was poured into sat. aq. NH₄Cl (20 mL) and extracted with EtOAc (3×10 mL). The combined organics were dried over anhydrous Na₂SO₄, filtered, and concentrated under reduced pressure to provide a residue that was used directly. LCMS: m/z=279.9, 281.9 [M−H]⁻.

Methyl 4-bromo-2-(3-cyanooxetan-3-yl)benzoate: To a mixture of 4-bromo-2-(3-cyanooxetan-3-yl)benzoic acid (3.2 g, 11.3 mmol) and Na₂CO₃ (3.6 g, 34 mmol) in DMF (50 mL) was added Mel (3.2 g, 22.7 mmol, 1.41 mL). The reaction mixture was stirred at 25° C. for 12 h. The reaction mixture was poured into ice-cold water (150 mL) and extracted with MTBE (3×50 mL). The combined organics were washed with brine (3×50 mL), dried over anhydrous Na₂SO₄, filtered, and concentrated under reduced pressure. The residue was purified by silica gel chromatography. ¹H NMR (400 MHz, CDCl₃): δ 8.00 (d, J=8.4 Hz, 1H), 7.65 (dd, J=1.6, 8.4 Hz, 1H), 7.44 (d, J=1.6 Hz, 1H), 5.28 (d, J=6.8 Hz, 2H), 4.98 (d, J=6.4 Hz, 2H), 3.93 (s, 3H).

6-bromospiro[2,3-dihydroisoquinoline-4,3′-oxetane]-1-one: To a solution of methyl 4-bromo-2-(3-cyanooxetan-3-yl)benzoate (500 mg, 1.69 mmol) and dichlorocobalt (219 mg, 1.69 mmol) in MeOH (5 mL) at 0° C. was added NaBH₄ (160 mg, 4.22 mmol). The reaction mixture was stirred at 20° C. for 3 h. The reaction mixture was poured into sat. aq. NH₄Cl (20 mL) and extracted with EtOAc (3×10 mL). The combined organics were washed with brine (2×10 mL), dried over anhydrous Na₂SO₄, filtered, and concentrated under reduced pressure. The residue was purified by silica gel chromatography. ¹H NMR (400 MHz, CDCl₃): δ 8.00 (d, J=8.4 Hz, 1H), 7.91 (d, J=2.0 Hz, 1H), 7.60 (dd, J=2.0, 8.4 Hz, 1H), 6.21 (br s, 1H), 4.85 (d, J=6.4 Hz, 2H), 4.68 (d, J=6.8 Hz, 2H), 3.90 (d, J=2.8 Hz, 2H).

Methyl 2-(6-bromo-1-oxospiro[3H-isoquinoline-4,3′-oxetane]-2-yl)acetate: To a solution of 6-bromospiro[2,3-dihydroisoquinoline-4,3′-oxetane]-1-one (300 mg, 1.12 mmol) in DMF (4 mL) at 0° C. was added methyl 2-bromoacetate (342 mg, 2.24 mmol) and NaH (67.1 mg, 1.68 mmol, 60% purity). The reaction mixture was stirred at 20° C. for 3 h. The reaction mixture was poured into sat. aq. NH₄Cl (20 mL) and extracted with EtOAc (3×10 mL). The combined organics were washed with brine (10 mL), dried over anhydrous Na₂SO₄, filtered, and concentrated under reduced pressure. The residue was purified by silica gel chromatography. ¹H NMR (400 MHz, CDCl₃): δ 8.01 (d, J=8.4 Hz, 1H), 7.89 (d, J=1.6 Hz, 1H), 7.58 (dd, J=1.6, 8.0 Hz, 1H), 4.86 (d, J=6.8 Hz, 2H), 4.73 (d, J=6.8 Hz, 2H), 4.38 (s, 2H), 3.99 (s, 2H), 3.79 (s, 3H).

2-(6-bromo-1-oxospiro[3H-isoquinoline-4,3′-oxetane]-2-yl)-N-pyrimidin-2-ylacetamide: To a solution of methyl 2-(6-bromo-1-oxospiro[3H-isoquinoline-4,3′-oxetane]-2-yl)acetate (150 mg, 0.44 mmol) in toluene (2.0 mL) were added pyrimidin-2-amine (50 mg, 0.53 mmol) and DABAL-Me₃ (170 mg, 0.66 mol). The reaction mixture was stirred at 60° C. for 4 h. The reaction mixture was poured into water (10 mL) and extracted with EtOAc (3×5 mL). The combined organics were washed with brine (5 mL), dried over anhydrous Na₂SO₄, filtered, and concentrated under reduced pressure. The residue was purified by preparatory reverse-phase HPLC. LCMS: m/z=402.9, 405.0 [M+H]⁺. ¹H NMR (400 MHz, CDCl₃): δ 8.84 (br s, 1H), 8.62 (d, J=4.8 Hz, 2H), 8.03 (d, J=8.4 Hz, 1H), 7.91 (d, J=2.0 Hz, TH), 7.59 (dd, J=1.6, 8.4 Hz, 1H), 7.05 (t, J=4.8 Hz, 1H), 4.89 (br s, 2H), 4.87 (d, J=6.8 Hz, 2H), 4.78 (d, J=6.4 Hz, 2H), 4.07 (s, 2H).

### Example 93

### 2-[6-(1-cyanocyclopropyl)-1-oxospiro[3H-isoquinoline-4,1′-cyclopropane]-2-yl]-N-(5-fluoropyrimidin-2-yl)acetamide (93)

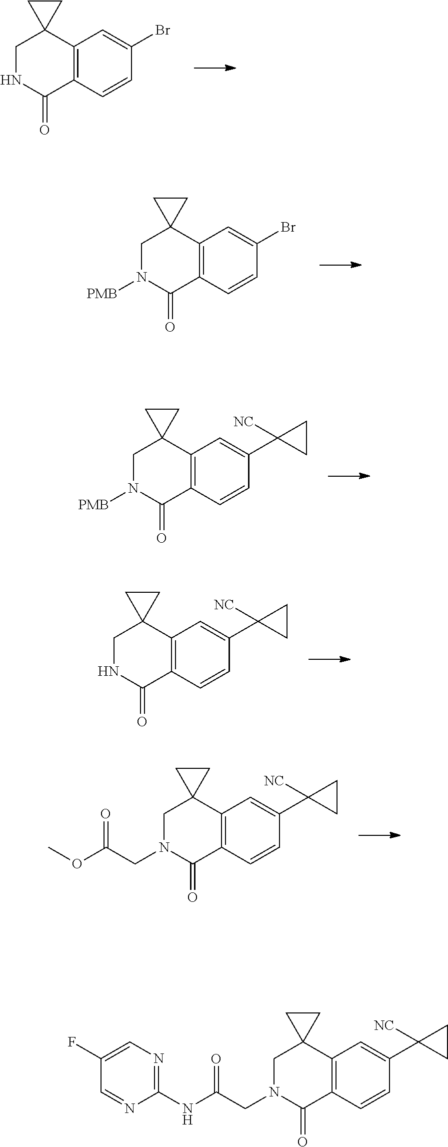

6-bromo-2-[(4-methoxyphenyl)methyl]spiro[3H-isoquinoline-4,1′-cyclopropane]-1-one: To a solution of 6-bromospiro[2,3-dihydroisoquinoline-4,1′-cyclopropane]-1-one (5.0 g, 19.8 mmol) in THF (100 mL) at 0° C. was added 4-methoxybenzyl chloride (3.7 g, 23.8 mmol) and NaH (1.6 g, 39.7 mmol, 60% purity). The reaction mixture was stirred at 50° C. for 12 h. The reaction mixture was poured into sat. aq. NH₄Cl (100 mL) and extracted with EtOAc (3×50 mL). The combined organics were washed with brine (50 mL), dried over anhydrous Na₂SO₄, filtered, and concentrated under reduced pressure. The residue was purified by silica gel chromatography. ¹H NMR (400 MHz, CDCl₃): δ 8.05 (d, J=8.0 Hz, 1H), 7.45 (dd, J=2.0, 8.4 Hz, 1H), 7.24 (d, J=8.4 Hz, 2H), 6.95 (d, J=1.6 Hz, 1H), 6.89-6.85 (m, 2H), 4.71 (s, 2H), 3.81 (s, 3H), 3.20 (s, 2H), 1.04-0.99 (m, 2H), 0.77-0.72 (m, 2H).

1-[2-[(4-methoxyphenyl)methyl]-1-oxospiro[3H-isoquinoline-4,1′-cyclopropane]-6-yl]cyclopropane-1-carbonitrile: To a solution of 6-bromo-2-[(4-methoxyphenyl)methyl]spiro[3H-isoquinoline-4,1′-cyclopropane]-1-one (1.0 g, 2.69 mmol) and cyclopropanecarbonitrile (270 mg, 4.03 mmol) in toluene (5.0 mL) was added Pd₂(dba)₃ (246 mg, 0.27 mmol) and BINAP (167 mg, 0.27 mmol). The reaction mixture was stirred at 20° C. for 30 min then LiHMDS (4.03 mL, 1 M in THF) was added. The reaction mixture was stirred at 80° C. for 3 h. The reaction mixture was poured into sat. aq. NH₄Cl (15 mL) and extracted with EtOAc (3×10 mL). The combined organics were washed with brine (10 mL), dried over anhydrous Na₂SO₄, filtered, and concentrated under reduced pressure. The residue was purified by silica gel chromatography. ¹H NMR (400 MHz, CDCl₃): δ 8.15 (d, J=8.0 Hz, 1H), 7.25 (d, J=8.4 Hz, 2H), 7.06 (dd, J=2.0, 8.4 Hz, 1H), 6.88-6.85 (m, 3H), 4.71 (s, 2H), 3.81 (s, 3H), 3.21 (s, 2H), 1.80-1.75 (m, 2H), 1.46-1.42 (m, 2H), 1.07-1.03 (m, 2H), 0.77-0.73 (m, 2H).

1-(1-oxospiro[2,3-dihydroisoquinoline-4,1′-cyclopropane]-6-yl)cyclopropane-1-carbonitrile: A solution of 1-[2-[(4-methoxyphenyl)methyl]-1-oxospiro[3H-isoquinoline-4,1-cyclopropane]-6-yl]cyclopropane-1-carbonitrile (730 mg, 2.04 mmol) in TFA (10 mL) was stirred at 70° C. for 20 h. The reaction mixture was cooled to ambient temperature, adjusted to pH=7 with sat. aq. Na₂CO₃, and extracted with EtOAc (3×10 mL). The combined organics were washed with brine (30 mL), dried over anhydrous Na₂SO₄, filtered, and concentrated under reduced pressure. The residue was purified by silica gel chromatography. ¹H NMR (400 MHz, CDCl₃): δ 8.08 (d, J=8.4 Hz, 1H), 7.05 (dd, J=1.6, 8.4 Hz, 1H), 6.90 (d, J=1.6 Hz, 1H), 6.67 (br s, 1H), 3.37 (d, J=2.4 Hz, 2H), 1.82-1.76 (m, 2H), 1.48-1.43 (m, 2H), 1.16-1.11 (m, 2H), 1.05-0.98 (m, 2H).

Methyl 2-[6-(1-cyanocyclopropyl)-1-oxospiro[3H-isoquinoline-4,1′-cyclopropane]-2-yl]acetate: To a solution of 1-(1-oxospiro[2,3-dihydroisoquinoline-4,1′-cyclopropane]-6-yl)cyclopropane-1-carbonitrile (200 mg, 0.84 mmol) in DMF (2 mL) at 0° C. was added NaH (50 mg, 1.26 mmol, 60% purity). The mixture was stirred at 0° C. for 30 min then methyl 2-bromoacetate (257 mg, 1.68 mmol) was added. The reaction mixture was stirred at 20° C. for 3 h. The reaction mixture was poured into sat. aq. NH₄Cl (10 mL) and extracted with EtOAc (3×5 mL). The combined organics were washed with brine (5 mL), dried over anhydrous Na₂SO₄, filtered, and concentrated under reduced pressure. The residue was purified by silica gel chromatography. ¹H NMR (400 MHz, CDCl₃): δ 8.10 (d, J=8.0 Hz, 1H), 7.04 (dd, J 1.6, 8.4 Hz, 1H), 6.90 (d, J 1.6 Hz, 1H), 4.35 (s, 2H), 3.76 (s, 3H), 3.45 (s, 2H), 1.82-1.76 (m, 2H), 1.48-1.43 (m, 2H), 1.19-1.14 (m, 2H), 1.08-1.04 (m, 2H).

2-[6-(1-cyanocyclopropyl)-1-oxospiro[3H-isoquinoline-4,1′-cyclopropane]-2-yl]-N-(5-fluoropyrimidin-2-yl)acetamide: To a solution of methyl 2-[6-(1-cyanocyclopropyl)-1-oxospiro[3H-isoquinoline-4,1′-cyclopropane]-2-yl]acetate (130 mg, 0.42 mmol) and 5-fluoropyrimidin-2-amine (142 mg, 1.26 mmol) in toluene (2 mL) was added DABAL-Me₃ (322 mg, 1.26 mmol). The reaction mixture was stirred at 60° C. for 3 h. The reaction mixture was cooled to ambient temperature, poured into water (10 mL), and extracted with EtOAc (3×5 mL). The combined organic layers were washed with brine (5 mL), dried over anhydrous Na₂SO₄, filtered, and concentrated under reduced pressure. The residue was purified by preparatory reverse-phase HPLC. LCMS: m/z=392.2 [M+H]⁺. 1H NMR (400 MHz, CDCl₃): δ 9.10 (br s, 1H), 8.49 (s, 2H), 8.13 (d, J=8.4 Hz, 1H), 7.07 (dd, J=1.6, 8.0 Hz, 1H), 6.90 (d, J=2.0 Hz, 1H), 4.59 (br s, 2H), 3.54 (s, 2H), 1.84-1.77 (m, 2H), 1.50-1.43 (m, 2H), 1.22-1.14 (m, 2H), 1.11-1.04 (m, 2H).

### Example 94

### 2-(6-chloro-1-oxospiro[3H-isoquinoline-4,1′-cyclopropane]-2-yl)-N-(5-fluoropyrimidin-2-yl)acetamide (94)

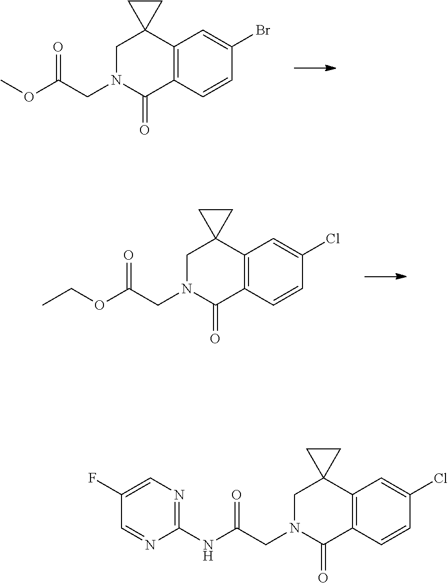 [SMILES: COC(=O)CN1CC2(CC2)c2cc(Br)ccc2C1=O ; CCOC(=O)CN1CC2(CC2)c2cc(Cl)ccc2C1=O ; O=C(CN1CC2(CC2)c2cc(Cl)ccc2C1=O)Nc1ncc(F)cn1]

Ethyl 2-(6-chloro-1-oxo-spiro[3H-isoquinoline-4,1′-cyclopropane]-2-yl)acetate: To a solution of methyl 2-(6-bromo-1-oxo-spiro[3H-isoquinoline-4,1′-cyclopropane]-2-yl)acetate (100 mg, 0.31 mmol) in EtOH (2.0 mL) were added L-proline (14 mg, 0.12 mmol), tetramethylammonium chloride (135 mg, 1.23 mmol), and Cu₂O (9 mg, 0.06 mmol). The reaction mixture was stirred at 110° C. for 12 h. The reaction mixture was quenched by addition of water (4 mL) and extracted with EtOAc (4×3 mL). The combined organics were washed with brine (4 mL), dried over anhydrous Na₂SO₄, and concentrated under reduced pressure. The residue was purified by preparatory TLC. LCMS: m/z=294.1, 296.1 [M+H]⁺.

2-(6-chloro-1-oxospiro[3H-isoquinoline-4,1′-cyclopropane]-2-yl)-N-(5-fluoropyrimidin-2-yl)acetamide: To a solution of ethyl 2-(6-chloro-1-oxo-spiro[3H-isoquinoline-4,1′-cyclopropane]-2-yl)acetate (80 mg, 0.27 mmol) and 5-fluoropyrimidin-2-amine (62 mg, 0.54 mmol) in DCE (1.0 mL) was added AlMe₃ (0.27 mL, 2 M in toluene). The reaction mixture was stirred at 60° C. for 2 h. The reaction mixture was quenched by addition of water (3 mL) and extracted with EtOAc (3×3 mL). The combined organics were washed with brine (3 mL), dried over anhydrous Na₂SO₄, and concentrated under reduced pressure. The residue was purified by preparatory reverse-phase HPLC. LCMS: m/z=361.1, 363.1 [M+H]⁺. H NMR (400 MHz, CDCl₃) δ 9.01 (br s, 1H), 8.49 (s, 2H), 8.10 (d, J=8.4 Hz, 1H), 7.30 (dd, J=8.4, 2.0 Hz, 1H), 6.84 (d, J=2.0 Hz. 1H), 4.58 (s, 2H), 3.55 (s, 2H), 1.16-1.12 (m, 2H), 1.10-1.06 (m, 2H).

### Example 95

### 2-[6-bromo-4-(difluoromethyl)-1-oxo-3,4-dihydroisoquinolin-2-yl]-N-(5-fluoropyrimidin-2-yl)acetamide (95)

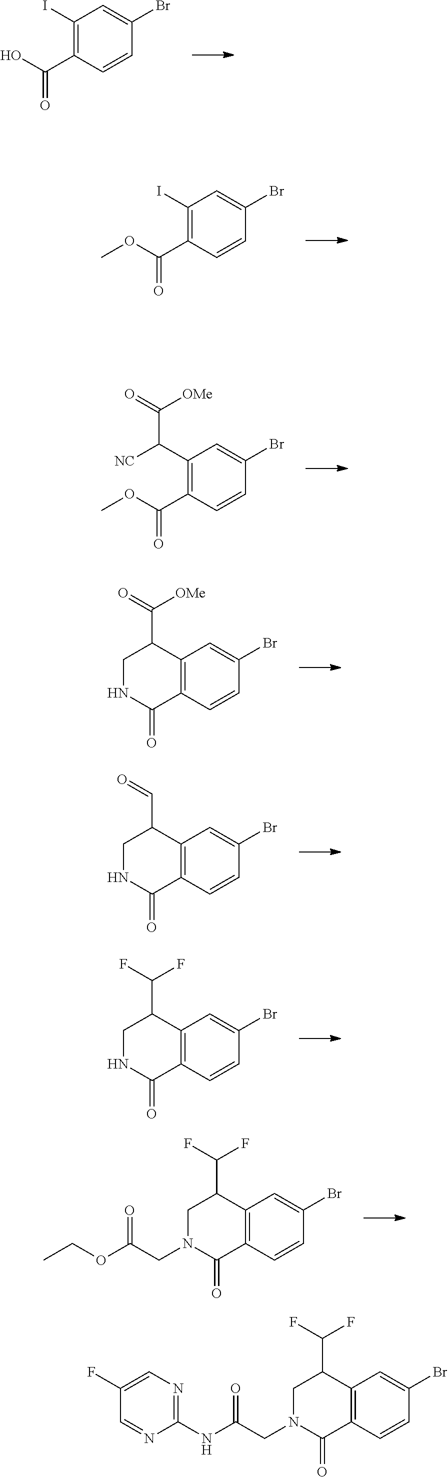

Methyl 4-bromo-2-iodobenzoate: To a solution of 4-bromo-2-iodobenzoic acid (14.5 g, 44.4 mmol) in MeOH (150 mL) was added conc. H₂SO₄ (21.8 g, 222 mmol). The reaction mixture was stirred at 80° C. for 6 h. The reaction mixture was poured into water (200 mL) and extracted with EtOAc (3×80 mL). The combined organics were washed with sat. aq. NaHCO₃ (3×50 mL), dried over anhydrous Na₂SO₄, filtered, and concentrated under reduced pressure to give an oil that was used directly.

Methyl 4-bromo-2-(1-cyano-2-methoxy-2-oxoethyl)benzoate: To a solution of methyl 4-bromo-2-iodobenzoate (10.0 g, 29.3 mmol) in 1,4-dioxane (100 mL) were added methyl 2-cyanoacetate (5.81 g, 58.7 mmol), Cs₂CO₃ (19.1 g, 58.7 mmol), CuI (1.12 g, 5.87 mmol), and pyridine-2-carboxylic acid (1.44 g, 11.7 mmol). The reaction mixture was degassed and purged with N₂ three times then stirred at 90° C. for 16 h. The reaction mixture was filtered. The filtrate was diluted with water (50 mL), adjusted to pH=4 with aq. HCl (2 M), and extracted with EtOAc (2×30 mL). The combined organics were washed with brine (2×30 mL), dried over anhydrous Na₂SO₄, filtered, and concentrated under reduced pressure. The residue was purified by silica gel column chromatography. ¹H NMR (400 MHz, CDCl₃): δ 7.98 (d, J=8.4 Hz, 1H), 7.80 (d, J=2.0 Hz, 1H), 7.65 (dd, J=2.0, 8.4 Hz, 1H), 5.95 (s, 1H), 3.92 (s, 3H), 3.85 (s, 3H).

Methyl 6-bromo-1-oxo-1,2,3,4-tetrahydroisoquinoline-4-carboxylate: To a solution of methyl 4-bromo-2-(1-cyano-2-methoxy-2-oxoethyl)benzoate (6.10 g, 19.5 mmol) in MeOH (60 mL) were added dichlorocobalt (2.54 g, 19.5 mmol) and NaBH₄ (2.9 g, 78.2 mmol). The mixture was stirred at 20° C. for 3 h. The reaction mixture was poured into sat. aq. NH₄Cl (50 mL) and extracted with EtOAc (3×30 mL). The combined organics were washed with brine (10 mL), dried over anhydrous Na₂SO₄, filtered, and concentrated under reduce pressure. The residue was purified by silica gel column chromatography. LCMS: m/z: 284.1, 286.0 [M+H]⁺.

6-bromo-1-oxo-1,2,3,4-tetrahydroisoquinoline-4-carbaldehyde: To a solution of methyl 6-bromo-1-oxo-1,2,3,4-tetrahydroisoquinoline-4-carboxylate (600 mg, 2.11 mmol) in DCM (10 mL) at −78° C. was added DIBAL (1 M in toluene, 4.22 mL). The reaction mixture was stirred at −78° C. for 2 h. The reaction mixture was quenched by addition aq. HCl (2 M, 10 mL) at −78° C., diluted with water (10 mL), and extracted with DCM (3×15 mL). The combined organics were washed with brine (30 mL), dried over anhydrous Na₂SO₄, filtered, and concentrated under reduced pressure to give a residue that was used directly. LCMS: m/z: 254.0, 256.0 [M+H]⁺.

6-bromo-4-(difluoromethyl)-3,4-dihydroisoquinolin-1(2H)-one: To a solution of 6-bromo-1-oxo-1,2,3,4-tetrahydroisoquinoline-4-carbaldehyde (500 mg, 1.97 mmol) in DCM (5 mL) was added DAST (317 mg, 1.97 mmol). The reaction mixture was stirred at 20° C. for 16 h. The reaction mixture was poured into water (10 mL) and extracted with DCM (3×5 mL). The combined organics were washed with brine (15 mL), dried over anhydrous Na₂SO₄, filtered, and concentrated under reduce pressure to give a residue that was used directly. LCMS: m/z: 275.9, 277.9 [M+H]⁺.

Ethyl 2-(6-bromo-4-(difluoromethyl)-1-oxo-3,4-dihydroisoquinolin-2(1H)-yl)acetate: To a solution of 6-bromo-4-(difluoromethyl)-3,4-dihydroisoquinolin-1(2H)-one (340 mg, 1.23 mmol) in DMF (5 mL) at 0° C. were added Cs₂CO₃ (401 mg, 1.23 mmol) and ethyl 2-iodoacetate (343 mg, 1.60 mmol). The reaction mixture was stirred at 20° C. for 2 h. The reaction mixture was quenched with sat. aq. NH₄Cl (10 mL) and extracted with EtOAc (3×5 mL). The combined organics were dried over anhydrous Na₂SO₄, filtered, and concentrated under reduced pressure. The residue was purified by silica gel chromatography. LCMS: m/z: 362.2, 364.2 [M+H]⁺.

2-(6-bromo-4-(difluoromethyl)-1-oxo-3,4-dihydroisoquinolin-2(1H)-yl)-N-(5-fluoropyrimidin-2-yl)acetamide: To a solution of ethyl 2-(6-bromo-4-(difluoromethyl)-1-oxo-3,4-dihydroisoquinolin-2(1H)-yl)acetate (85 mg, 0.24 mmol) in toluene (1.0 mL) were added 5-fluoropyrimidin-2-amine (53 mg, 0.47 mmol) and AlMe₃ (2 M in toluene, 0.35 mL). The reaction mixture was stirred at 90° C. for 3 h. The reaction mixture was diluted with water (5 mL) and extracted with EtOAc (3×2 mL). The combined organics were washed with brine (5 mL), dried over anhydrous Na₂SO₄, filtered, and concentrated under reduced pressure. The residue was purified by preparatory reverse-phase HPLC. LCMS: m/z=428.9, 430.9 [M+H]⁺. ¹H NMR (400 MHz, CDCl₃): δ 8.64 (br s, 1H), 8.48 (s, 2H), 8.03 (d, J=8.0 Hz, 1H), 7.63 (dd, J=2.0, 8.4 Hz, 1H), 7.52 (s, 1H), 6.15 (dt, J=6.4, 56.0 Hz, 1H), 4.75 (s, 2H), 4.14 (br d, J=12.4 Hz, 1H), 3.73 (br d, J=13.2 Hz, 1H), 3.34-3.21 (m, 1H).

### Example 96

### 2-[6-(difluoromethyl)-1-oxospiro[3H-isoquinoline-4,1′-cyclopropane]-2-yl]-N-(5-fluoropyrimidin-2-yl)acetamide (96)

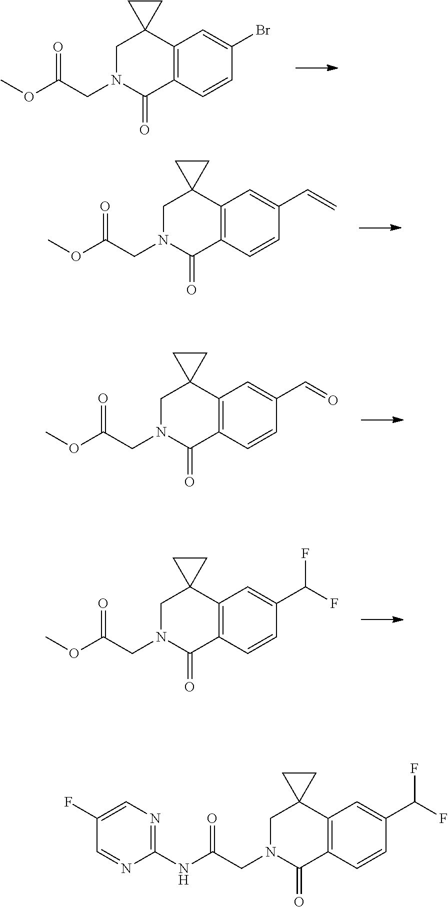

Methyl 2-(1-oxo-6-vinyl-spiro[3H-isoquinoline-4,1′-cyclopropane]-2-yl)acetate: To a solution of methyl 2-(6-bromo-1-oxo-spiro[3H-isoquinoline-4,1-cyclopropane]-2-yl)acetate (1.0 g, 3.08 mmol) in 1,4-dioxane (30 mL) were added potassium trifluoro(vinyl)borate (2.1 g, 15.4 mmol), CsF (1.4 g, 9.25 mmol), and Pd(dppf)Cl₂ (226 mg, 0.31 mmol). The reaction mixture was stirred at 90° C. for 3 h. The reaction mixture was cooled to ambient temperature and concentrated under reduced pressure. The residue was purified by silica gel chromatography. LCMS: m/z=272.1 [M+H]⁺.

Methyl 2-(6-formyl-1-oxo-spiro[3H-isoquinoline-4,1′-cyclopropane]-2-yl)acetate: A solution of methyl 2-(1-oxo-6-vinyl-spiro[3H-isoquinoline-4,1′-cyclopropane]-2-yl)acetate (800 mg, 2.95 mmol) in DCM (20 mL) was stirred at −78° C. for 0.5 h under ozone (15 psi). To the reaction mixture was added Me₂S (2.1 g, 34.1 mmol) and the reaction mixture was stirred at 20° C. for 15 h. The reaction mixture was diluted with water (30 mL) and extracted with DCM (3×10 mL). The combined organics were washed with brine (20 mL), dried over anhydrous Na₂SO₄, filtered, and concentrated under reduced pressure. The residue was purified by silica gel chromatography. LCMS: m/z=274.1 [M+H]⁺.

Methyl 2-(6-difluoromethyl-1-oxo-spiro[3H-isoquinoline-4,1′-cyclopropane]-2-yl)acetate: Methyl 2-(6-formyl-1-oxo-spiro[3H-isoquinoline-4,1′-cyclopropane]-2-yl)acetate (200 mg, 0.73 mmol) was added to bis(2-methoxyethyl)aminosulfur trifluoride (2.02 g, 9.13 mmol) at 0° C. The reaction mixture was stirred at 20° C. for 16 h. The reaction mixture was poured into sat. aq. NaHCO₃ (5 mL) and extracted with DCM (3×2 mL). The combined organics were washed with brine (5 mL), dried over anhydrous Na₂SO₄, filtered, and concentrated under reduced pressure. The residue was purified by silica gel chromatography. LCMS: m/z=296.2 [M+H]⁺.

2-[6-(difluoromethyl)-1-oxospiro[3H-isoquinoline-4,1′-cyclopropane]-2-yl]-N-(5-fluoropyrimidin-2-yl)acetamide: To a solution of methyl 2-(6′-(difluoromethyl)-1′-oxo-1′H-spiro[cyclopropane-1,4′-isoquinolin]-2′(3′H)-yl)acetate (80 mg, 0.27 mmol) in DCE (1.0 mL) were added 5-fluoropyrimidin-2-amine (92 mg, 0.81 mmol) and AlMe₃ (2 M in toluene, 0.41 mL). The reaction mixture was stirred at 60° C. for 3 h. The reaction mixture was diluted with water (5 mL) and extracted with EtOAc (3×2 mL). The combined organics were washed with brine (5 mL), dried over anhydrous Na₂SO₄, filtered, and concentrated under reduced pressure. The residue was purified by preparatory reverse-phase HPLC. LCMS: m/z=377.1 [M+H]⁺. ¹H NMR (400 MHz, CDCl₃): δ 9.02 (s, 1H), 8.50 (s, 2H), 8.25 (d, J=8.0 Hz, 1H), 7.46 (d, J=8.0 Hz, 1H), 7.02 (s, 1H), 6.65 (t, J=56.4 Hz, 1H), 4.62 (s, 2H). 3.58 (s, 2H), 1.23-1.14 (m, 2H), 1.14-1.06 (m, 2H).

### Example 97

### N-(5-fluoropyrimidin-2-yl)-2-[6-(1-methylcyclopropyl)-1-oxospiro[3H-isoquinoline-4,1′-cyclopropane]-2-yl]acetamide (97)

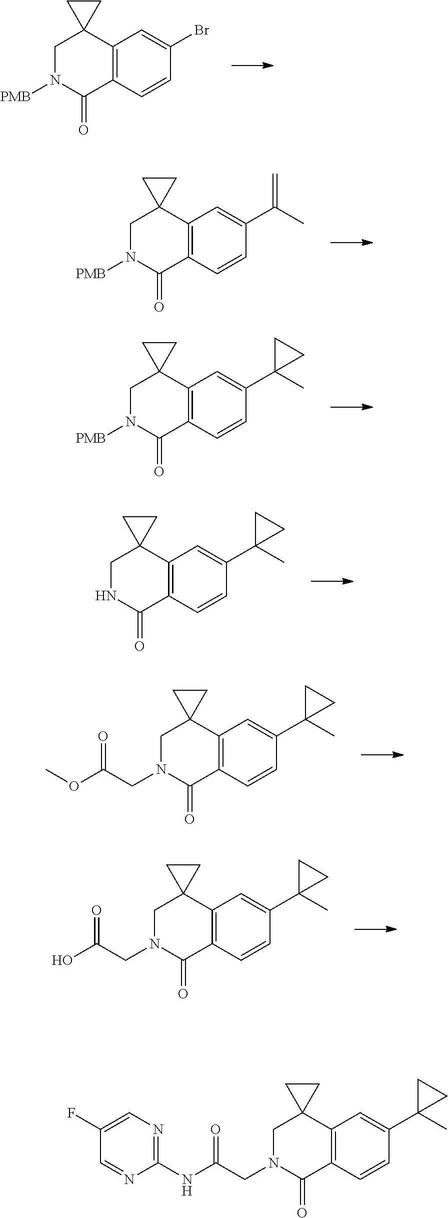

2-(4-methoxyphenyl)methyl-6-prop-1-en-2-ylspiro[3H-isoquinoline-4,1′-cyclopropane]-1-one: To a solution of 6-bromo-2-[(4-methoxyphenyl)methyl]spiro[3H-isoquinoline-4,1′-cyclopropane]-1-one (2.00 g, 5.37 mmol) and 2-isopropenyl-4,4,5,5-tetramethyl-1,3,2-dioxaborolane (1.35 g, 8.06 mmol) in 1,4-dioxane (20 mL) and water (2 mL) were added Pd(dppf)Cl₂ (393 mg, 0.54 mmol) and Cs₂CO₃ (4.38 g, 13.4 mmol). The reaction mixture was stirred at 90° C. for 3 h. The reaction mixture was poured into water (20 mL) and extracted with EtOAc (3×10 mL). The combined organics were washed with brine (10 mL), dried over anhydrous Na₂SO₄, filtered, and concentrated under reduced pressure. The residue was purified by silica gel column chromatography. ¹H NMR (400 MHz, CDCl₃): δ 8.15 (d, J=8.0 Hz, 1H), 7.41 (dd, J=1.6, 8.0 Hz, 1H), 7.25 (d, J=8.0 Hz, 2H), 6.89-6.84 (m, 3H), 5.39 (s, 1H), 5.15 (s, 1H), 4.72 (s, 2H), 3.80 (s, 3H), 3.9 (s, 2H), 2.14 (s, 3H), 1.07-1.00 (m, 2H), 0.76-0.68 (m, 2H).

2-(4-methoxyphenyl)methyl-6-(1-methylcyclopropyl)spiro[3H-isoquinoline-4,1′-cyclopropane]-1-one: To a solution of 2-(4-methoxyphenyl)methyl-6-prop-1-en-2-ylspiro[3H-isoquinoline-4,1′-cyclopropane]-1-one (1.20 g, 3.60 mmol) in CH₂I₂ (15 mL, 49.8 g, 186 mmol) 0° C. were added ZnEt₂ (15 mL, 1M in toluene). The reaction mixture was stirred at 50° C. for 12 h. The reaction mixture was quenched with water (50 mL) and extracted with EtOAc (3×20 mL). The combined organics were washed with brine (30 mL), dried over anhydrous Na₂SO₄, filtered, and concentrated under reduced pressure. The residue was purified by silica gel column chromatography. LCMS: m/z=348.4 [M+H]⁺.

6-(1-methylcyclopropyl)spiro[2,3-dihydroisoquinoline-4,1′-cyclopropane]-1-one: A solution of 2-(4-methoxyphenyl)methyl-6-(1-methylcyclopropyl)spiro[3H-isoquinoline-4,1′-cyclopropane]-1-one (550 mg, 1.58 mmol) in TFA (10 mL) was stirred at 60° C. for 1 h. The reaction mixture was quenched with sat. aq. NaHCO₃ (20 mL) and extracted with EtOAc (3×10 mL). The combined organics were washed with brine (10 mL), dried over anhydrous Na₂SO₄, filtered, and concentrated under reduced pressure. The residue was purified by silica gel column chromatography. ¹H NMR (400 MHz, CDCl₃): δ 8.02 (d, J=8.0 Hz, 1H), 7.18 (dd, J=1.6, 8.0 Hz, 1H), 6.72 (d, J=1.6 Hz, 1H), 6.23 (br s, 1H), 3.37-3.35 (m, 2H), 1.41 (s, 3H), 1.13-1.07 (m, 2H), 1.00-0.95 (m, 2H), 0.91-0.86 (m, 2H), 0.81-0.76 (m, 2H).

Methyl 2-[6-(1-methylcyclopropyl)-1-oxospiro[3H-isoquinoline-4,1′-cyclopropane]-2-yl]acetate: To a solution of 6-(1-methylcyclopropyl)spiro[2,3-dihydroisoquinoline-4,1′-cyclopropane]-1-one (200 mg, 0.88 mmol) in DMF (5 mL) were added NaH (52 mg, 1.32 mmol, 60% purity) and methyl 2-bromoacetate (162 mg, 1.06 mmol). The reaction mixture was stirred at 20° C. for 2 h. The reaction mixture was poured into sat. aq. NH₄Cl (5 mL) and extracted with EtOAc (3×2 mL). The combined organics were washed with brine (2 mL), dried over anhydrous Na₂SO₄, filtered, and concentrated under reduced pressure. The residue was purified by silica gel column chromatography. ¹H NMR (400 MHz, CDCl₃): δ 8.03 (d, J=8.0 Hz, 1H), 7.19-7.14 (m, 1H), 6.72-6.69 (m, 1H), 4.34 (s, 2H), 3.75 (s, 3H), 3.43 (s, 2H), 1.42 (s, 3H), 1.14-1.10 (m, 2H), 1.03-0.98 (m, 2H), 0.91-0.86 (m, 2H), 0.80-0.76 (m, 2H).

2-[6-(1-methylcyclopropyl)-1-oxospiro[3H-isoquinoline-4,1′-cyclopropane]-2-yl]acetic acid: To a solution of methyl 2-[6-(1-methylcyclopropyl)-1-oxospiro[3H-isoquinoline-4,1′-cyclopropane]-2-yl]acetate (100 mg, 0.33 mmol) in THF (2 mL) and water (2 mL) was added LiOH·H₂O (35 mg, 0.84 mmol). The reaction mixture was stirred at 20° C. for 2 h. The reaction mixture was adjusted to pH=4 with aq. HCl (3 N) and extracted with EtOAc (2×15 mL). The combined organics were washed with brine (15 mL), dried over anhydrous Na₂SO₄, filtered, and concentrated under reduced pressure to provide a solid that was used directly. LCMS: m/z=286.2 [M+H]⁺. H NMR (400 MHz, CDCl₃): δ 8.02 (d, J=8.0 Hz, 1H), 7.21-7.13 (m, 1H), 6.71 (s, 1H), 4.35 (s, 2H), 3.46 (s, 2H), 1.40 (s, 3H), 1.15-1.10 (m, 2H), 1.04-0.98 (m, 2H), 0.91-0.85 (m, 2H), 0.81-0.77 (m, 2H).

N-(5-fluoropyrimidin-2-yl)-2-[6-(1-methylcyclopropyl)-1-oxospiro[3H-isoquinoline-4,1′-cyclopropane]-2-yl]acetamide: To a solution of 2-[6-(1-methylcyclopropyl)-1-oxospiro[3H-isoquinoline-4,1′-cyclopropane]-2-yl]acetic acid (50 mg, 0.18 mmol) and 5-fluoropyrimidin-2-amine (24 mg, 0.21 mmol) in pyridine (1.0 mL) was added EDCI (50 mg, 0.26 mmol). The reaction mixture was stirred at 20° C. for 16 h. The reaction mixture was quenched with sat. aq. NH₄Cl (5 mL) and extracted with EtOAc (3×2 mL). The combined organics were washed with brine (5 mL), dried over anhydrous Na₂SO₄, filtered, and concentrated under reduced pressure. The residue was purified by preparatory reverse-phase HPLC. LCMS: m/z=381.1 [M+H]⁺. ¹H NMR (400 MHz, CDCl₃): 9.19 (br s, 1H), 8.52 (s, 2H), 8.08 (d, J=8.0 Hz, 1H). 7.23-7.15 (m, 1H), 6.71 (s. 1H), 4.51 (s, 2H), 3.51 (s, 2H), 1.41 (s, 3H), 1.16-1.11 (m, 2H), 1.05-0.99 (m, 2H), 0.91-0.86 (m, 2H), 0.81-0.77 (m, 2H).

### Example 98

### 2-(6-chloro-5-fluoro-1-oxospiro[3H-isoquinoline-4,1′-cyclopropane]-2-yl)-N-(5-fluoropyrimidin-2-yl)acetamide (98)

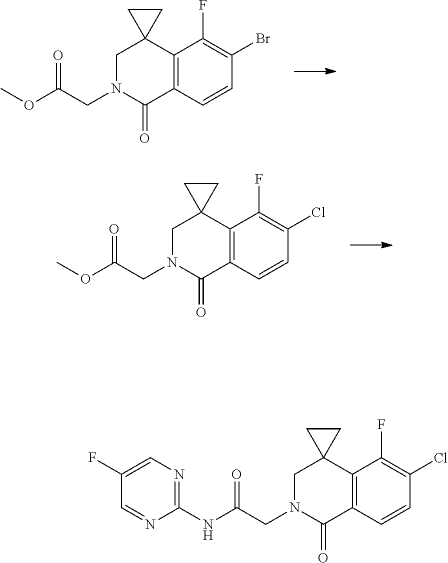 [SMILES: COC(=O)CN1CC2(CC2)c2c(ccc(Br)c2F)C1=O ; COC(=O)CN1CC2(CC2)c2c(ccc(Cl)c2F)C1=O ; O=C(CN1CC2(CC2)c2c(ccc(Cl)c2F)C1=O)Nc1ncc(F)cn1]

Methyl 2-(6-chloro-5-fluoro-1-oxospiro[3H-isoquinoline-4,1′-cyclopropane]-2-yl)acetate: To a solution of methyl 2-(6-bromo-5-fluoro-1-oxospiro[3H-isoquinoline-4,1′-cyclopropane]-2-yl)acetate (150 mg, 0.43 mmol) in EtOH (2.0 mL) were added Cu₂O (13 mg, 0.08 mmol), L-proline (20 mg, 0.18 mmol) and tetramethylammonium chloride (192 mg, 1.75 mmol). The reaction mixture was stirred at 70° C. for 12 h. The reaction mixture was cooled to ambient temperature, diluted with water (30 mL), and extracted with EtOAc (3×30 mL). The combined organics were washed with brine (30 mL), dried over anhydrous Na₂SO₄, filtered, and concentrated under reduced pressure. The residue was purified by preparatory TLC. LCMS: m/z=298.1, 300.1 [M+H]⁺.

2-(6-chloro-5-fluoro-1-oxospiro[3H-isoquinoline-4,1′-cyclopropane]-2-yl)-N-(5-fluoropyrimidin-2-yl)acetamide: To a solution of methyl 2-(6-chloro-5-fluoro-1-oxospiro[3H-isoquinoline-4,1′-cyclopropane]-2-yl)acetate (90 mg, 0.30 mmol) in toluene (3 mL) at 0° C. were added DABAL-Me₃ (78 mg, 0.30 mmol) and 5-fluoropyrimidin-2-amine (68 mg, 0.60 mmol). The reaction mixture was stirred at 60° C. for 2 h. The reaction mixture was poured into ice-cold H₂O (10 mL) and extracted with EtOAc (4×5 mL). The combined organics were washed with brine (10 mL), dried over anhydrous Na₂SO₄, filtered, and concentrated under reduced pressure. The residue was purified by preparatory reverse-phase HPLC. LCMS: m/z=379.0, 381.0 [M+H]⁺. ¹H NMR (400 MHz, MeOD): δ 8.58 (s, 2H), 7.83 (d, J=8.4 Hz, 1H), 7.44 (t, J=7.8 Hz, 1H), 4.59 (s, 2H), 3.52 (s, 2H), 1.60-1.56 (m, 2H), 1.17-1.12 (m, 2H).

### Example 99

### 2-(6-bromo-7-fluoro-1-oxospiro[3H-isoquinoline-4,1′-cyclopropane]-2-yl)-N-(5-fluoropyrimidin-2-yl)acetamide (99)

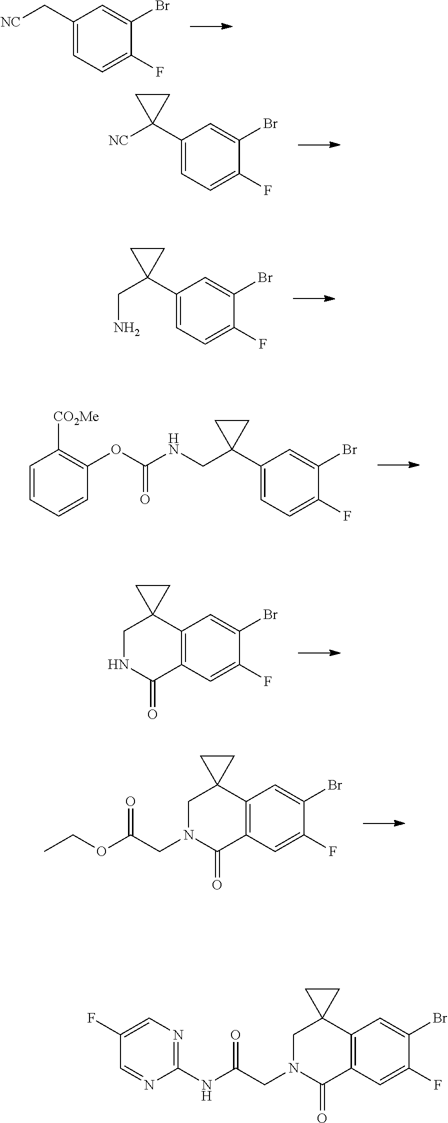

1-(3-bromo-4-fluorophenyl)cyclopropanecarbonitrile: To a solution of NaH (2.15 g, 53.7 mmol, 60% purity) in DMSO (50 mL) was added 2-(3-bromo-4-fluorophenyl)acetonitrile (5.0 g, 23.4 mmol) and 1-bromo-2-chloroethane (4.0 g, 28.1 mmol). The reaction mixture was stirred at 20° C. for 2 h. The reaction mixture was quenched with sat. aq. NH₄Cl (150 mL) and extracted with EtOAc (3×60 mL). The combined organics were washed with brine (60 mL), dried over anhydrous Na₂SO₄, filtered, and concentrated under reduced pressure. The residue was purified by silica gel column chromatography. ¹H NMR (400 MHz, CDCl₃): δ 7.49 (dd, J=4.0, 8.0 Hz, 1H), 7.27-7.22 (m, 1H), 7.15-7.08 (m, 1H), 1.81-1.69 (m, 2H), 1.44-1.32 (m, 2H).

(1-(3-bromo-4-fluorophenyl)cyclopropyl)methanamine: To a solution of 1-(3-bromo-4-fluorophenyl)cyclopropanecarbonitrile (3.0 g, 12.5 mmol) in MeOH (30 mL) at 0° C. were added NaBH₄ (946 mg, 25 mmol) and dichlorocobalt (1.62 g, 12.5 mmol). The reaction mixture was stirred at 20° C. for 3 h. The reaction mixture was quenched with sat. aq. NH₄Cl (50 mL) and extracted with EtOAc (3×30 mL). The combined organics were washed with brine (30 mL), dried over anhydrous Na₂SO₄, filtered, and concentrated under reduced pressure to provide a solid that was used directly. LCMS: m/z=244.1, 246.1 [M+H]⁺.

Methyl 2-((((1-(3-bromo-4-fluorophenyl)cyclopropyl)methyl)carbamoyl)oxy)benzoate: To a solution of (1-(3-bromo-4-fluorophenyl)cyclopropyl)methanamine (1.90 g, 6.23 mmol) in THF (20 mL) was added dimethyl 2,2′-(carbonylbis(oxy))dibenzoate (1.71 g, 5.19 mmol). The reaction mixture was stirred at 20° C. for 16 h. The reaction mixture was quenched with sat. aq. NH₄Cl (20 mL) and extracted with EtOAc (3×15 mL). The combined organics were washed with brine (15 mL), dried over anhydrous Na₂SO₄, filtered and concentrated under reduced pressure. The residue was purified by silica gel column chromatography.

6-bromo-7-fluoro-spiro[2,3-dihydroisoquinoline-4,1′-cyclopropane]-1-one: To a solution of methyl 2-((((1-(3-bromo-4-fluorophenyl)cyclopropyl)methyl)carbamoyl)oxy)benzoate (1.1 g, 2.61 mmol) in DCM (10 mL) was added TfOH (3.91 g, 26.1 mmol). The reaction mixture was stirred at 0° C. for 0.5 h. The mixture was quenched with sat. aq. Na₂CO₃ (20 mL) and extracted with EtOAc (3×15 mL). The combined organics were washed with brine (15 mL), dried over anhydrous Na₂SO₄, filtered, and concentrated under reduced pressure. The residue was purified by silica gel column chromatography. ¹H NMR (400 MHz, CDCl₃): δ 7.85 (d, J=8.0 Hz, 1H), 7.09-7.01 (m, 1H), 6.24 (br s, 1H), 3.37 (d, J=2.8 Hz, 2H), 1.11-1.00 (m, 4H).

Ethyl 2-(6′-bromo-7′-fluoro-1′-oxo-1′H-spiro[cyclopropane-1,4′-isoquinolin]-2′(3′H)-yl)acetate: To a solution of 6-bromo-7-fluorospiro[2,3-dihydroisoquinoline-4,1′-cyclopropane]-1-one (50 mg, 0.19 mmol) in DMF (2 mL) at 0° C. was added Cs₂CO₃ (120 mg, 0.37 mmol) and ethyl 2-iodoacetate (59 mg, 0.28 mmol). The reaction mixture was stirred at 20° C. for 2 h. The reaction mixture was quenched with sat. aq. NH₄Cl (10 mL) and extracted with EtOAc (3×5 mL). The combined organics were washed with brine (15 mL), dried over anhydrous Na₂SO₄, filtered, and concentrated under reduced pressure. The residue was purified by preparatory TLC. LCMS: m/z=356.0, 358.0 [M+H]⁺. ¹H NMR (400 MHz, CDCl₃): δ 7.86 (d, J=8.0 Hz, 1H), 7.03 (d, J=8.0 Hz, 1H), 4.30 (s, 2H), 4.26-4.19 (m, 2H), 3.45 (s, 2H), 1.32-1.27 (m, 3H), 1.10-1.05 (m, 4H).

2-(6-bromo-7-fluoro-1-oxospiro[3H-isoquinoline-4,1′-cyclopropane]-2-yl)-N-(5-fluoropyrimidin-2-yl)acetamide: To a solution of ethyl 2-(6-bromo-1-oxo-spiro[3H-isoquinoline-4,1′-cyclopropane]-2-yl)acetate (35 mg, 0.10 mmol) in toluene (1 mL) was added DABAL-Me₃ (25 mg, 0.10 mmol) and 5-fluoropyrimidin-2-amine (12 mg, 0.11 mmol). The reaction mixture was stirred at 60° C. for 3 h. The reaction mixture was quenched with sat. aq. NH₄Cl (5 mL) and extracted with EtOAc (3×2 mL). The combined organics were washed with brine (5 mL), dried over anhydrous Na₂SO₄, filtered, and concentrated under reduced pressure. The residue was purified by preparatory reverse-phase HPLC. LCMS: m/z=423.0, 425.0 [M+H]⁺. ¹H NMR (400 MHz, DMSO-d₆): δ 10.98 (br s, 1H), 8.80 (s, 2H), 7.70 (d, J=8.0 Hz, 1H), 7.43 (d, J=8.0 Hz, 1H), 4.51 (s, 2H), 3.49 (s, 2H), 1.20-1.12 (m, 2H), 1.08-1.01 (m, 2H).

### Example 100

### 2-(6-cyclopropyl-5-fluoro-1-oxospiro[3H-isoquinoline-4,1′-cyclopropane]-2-yl)-N-(5-fluoropyrimidin-2-yl)acetamide (100)

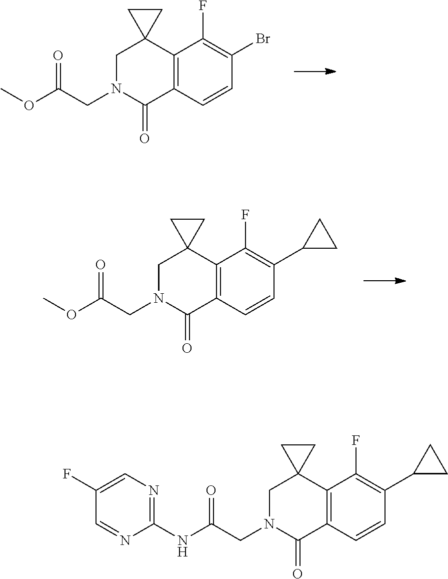 [SMILES: COC(=O)CN1CC2(CC2)c2c(ccc(Br)c2F)C1=O ; COC(=O)CN1CC2(CC2)c2c(ccc(C3CC3)c2F)C1=O ; O=C(CN1CC2(CC2)c2c(ccc(C3CC3)c2F)C1=O)Nc1ncc(F)cn1]

Methyl 2-(6-cyclopropyl-5-fluoro-1-oxo-spiro[3H-isoquinoline-4,1′-cyclopropane]-2-yl)acetate: To a solution of methyl 2-(6-bromo-5-fluoro-1-oxo-spiro[3H-isoquinoline-4,1′-cyclopropane]-2-yl)acetate (200 mg, 0.61 mmol) and cyclopropylboronic acid (151 mg, 1.75 mmol) in 1,4-dioxane (2.0 mL) were added CsF (266 mg, 1.75 mmol) and Pd(dppf)Cl₂ (43 mg, 0.06 mmol). The reaction mixture was stirred at 100° C. for 6 h. The reaction mixture was diluted with water (20 mL) and extracted with EtOAc (3×10 mL). The combined organics were washed with brine (10 mL), dried over anhydrous Na₂SO₄, filtered, and concentrated under reduced pressure. The residue was purified by silica gel column chromatography. ¹H NMR (400 MHz, CDCl₃): δ 7.84 (d, J=8.0 Hz, 1H), 6.84-6.67 (m, 1H), 4.29 (s, 2H), 3.76 (s, 3H), 3.32 (s, 2H), 2.12-2.05 (m, 1H), 1.67-1.61 (m, 2H), 1.06-0.99 (m, 2H), 0.99-0.93 (m, 2H), 0.78-0.71 (m, 2H).

2-(6-cyclopropyl-5-fluoro-1-oxospiro[3H-isoquinoline-4,1′-cyclopropane]-2-yl)-N-(5-fluoropyrimidin-2-yl)acetamide: To a solution of methyl 2-(6-cyclopropyl-5-fluoro-1-oxo-spiro[3H-isoquinoline-4,1′-cyclopropane]-2-yl)acetate (100 mg, 0.33 mmol) and 5-fluoropyrimidin-2-amine (75 mg, 0.66 mmol) in toluene (2.0 mL) was added DABAL-Me₃ (169 mg, 0.66 mmol). The reaction mixture was stirred at 60° C. for 12 h. The reaction mixture was diluted with water (5 mL) and extracted with EtOAc (3×10 mL). The combined organics were washed with brine (3 mL), dried over anhydrous Na₂SO₄, filtered, and concentrated under reduced pressure. The residue was purified by preparatory reverse-phase HPLC. LCMS: m/z=385.1 [M+H]⁺. ¹H NMR (400 MHz, CDCl₃): δ 9.12 (br s, 1H), 8.49 (s, 2H), 7.88 (d, J=8.0 Hz, 1H), 6.81-3.75 (m, 1H), 4.53 (s, 2H), 3.43 (s, 2H), 2.15-1.99 (m, 1H), 1.67-1.63 (m, 2H), 1.07-1.01 (m, 2H), 1.00-0.95 (m, 2H), 0.80-0.70 (m, 2H).

### Example 101

### 2-[6-(1,1-difluoroethyl)-1-oxospiro[3H-isoquinoline-4,1′-cyclopropane]-2-yl]-N-(5-fluoropyrimidin-2-yl)acetamide (101)

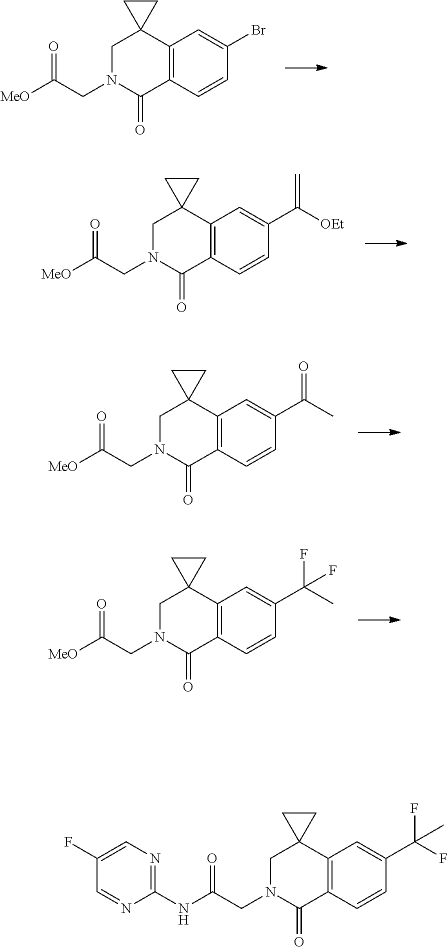 [SMILES: COC(=O)CN1CC2(CC2)c2cc(Br)ccc2C1=O ; C=C(OCC)c1ccc2c(c1)C1(CC1)CN(CC(=O)OC)C2=O ; COC(=O)CN1CC2(CC2)c2cc(C(C)=O)ccc2C1=O ; COC(=O)CN1CC2(CC2)c2cc(C(C)(F)F)ccc2C1=O ; CC(F)(F)c1ccc2c(c1)C1(CC1)CN(CC(=O)Nc1ncc(F)cn1)C2=O]

Methyl 2-(6-(1-ethoxyvinyl)-1-oxo-spiro[3H-isoquinoline-4,1′-cyclopropane]-2-yl)acetate: To a solution of methyl 2-(6-bromo-1-oxo-spiro[3H-isoquinoline-4,1′-cyclopropane]-2-yl)acetate (500 mg, 1.54 mmol) in DMF (5 mL) were added tributyl(1-ethoxyvinyl)stannane (2.80 g, 7.71 mmol) and Pd(PPh₃)₂Cl₂ (108 mg, 0.15 mmol). The reaction mixture was stirred at 100° C. for 2 h. The reaction mixture was poured into sat. aq. KF (30 mL) and extracted with EtOAc (3×15 mL). The combined organics were washed with brine (3×10 mL), dried over anhydrous Na₂SO₄, filtered, and concentrated under reduced pressure. The residue was purified by silica gel chromatography. LCMS: m/z=316.2 [M+H]⁺.

Methyl 2-(6-acetyl-1-oxo-spiro[3H-isoquinoline-4,1′-cyclopropane]-2-yl)acetate: A solution of methyl 2-(6-(1-ethoxyvinyl)-1-oxo-spiro[3H-isoquinoline-4,1′-cyclopropane]-2-yl)acetate (250 mg, 0.8 mmol) in HCl (10 mL, 4 M in EtOAc) was stirred at 20° C. for 2 h. The reaction mixture was concentrated under reduced pressure, and the residue was purified directly by silica gel chromatography. ¹H NMR (400 MHz, CDCl₃): δ 8.23 (d, J=8.0 Hz, 1H), 7.85 (dd, J=8.2, 1.6 Hz, 1H), 7.47 (d, J=1.6 Hz, 1H), 4.37 (s, 2H), 3.77 (s, 3H), 3.49 (s, 2H), 2.63 (s, 3H), 1.23-1.19 (m, 2H), 1.10-1.05 (m, 2H).

Methyl 2-(6-(1,1-difluoroethyl)-1-oxo-spiro[3H-isoquinoline-4,1′-cyclopropane]-2-yl)acetate: Methyl 2-(6-acetyl-1-oxo-spiro[3H-isoquinoline-4,1′-cyclopropane]-2-yl)acetate (220 mg, 0.77 mmol) was dissolved in BAST (5 mL) at 0° C. The reaction mixture was stirred at 80° C. for 5 h. The reaction mixture was poured into ice-cold sat. aq. NaHCO₃ (20 mL) and extracted with EtOAc (3×10 mL). The combined organics were dried over anhydrous Na₂SO₄, filtered, and concentrated under reduced pressure. The residue was purified by silica gel chromatography. ¹H NMR (400 MHz, CDCl₃): δ 8.20-8.16 (m, 1H), 7.45-7.40 (m, 1H), 6.99 (s, 1H), 4.36 (s, 2H), 3.77 (s, 3H), 3.47 (s, 2H), 1.98-1.84 (t, J=18.4 Hz, 3H), 1.18-1.14 (m, 2H), 1.09-1.04 (m, 2H).

2-[6-(1,1-difluoroethyl)-1-oxospiro[3H-isoquinoline-4,1′-cyclopropane]-2-yl]-N-(5-fluoropyrimidin-2-yl)acetamide: To a solution of methyl 2-(6-(1,1-difluoroethyl)-1-oxo-spiro[3H-isoquinoline-4,1′-cyclopropane]-2-yl)acetate (105 mg, 0.34 mmol) and 5-fluoropyrimidin-2-amine (115 mg, 1.02 mmol) in THF (1.0 mL) and toluene (2.0 mL) was added AlMe₃ (2 M in toluene, 0.5 mL). The reaction mixture was stirred at 90° C. for 3 h. The reaction mixture was poured into ice-cold water (10 mL) and extracted with EtOAc (3×5 mL). The combined organics were dried over anhydrous Na₂SO₄, filtered, and concentrated under reduced pressure. The residue was purified by preparatory reverse-phase HPLC. LCMS: m/z=391.2 [M+H]⁺. ¹H NMR (400 MHz, DMSO-d₆): δ 10.96 (br s, 1H), 8.77 (s, 2H), 7.99 (d, J=8.2 Hz, 1H), 7.51 (d, J=8.0 Hz, 1H), 7.15 (s, 1H), 4.52 (s, 2H), 3.52 (s, 2H), 1.98 (t, J=18.8 Hz, 3H), 1.20-1.13 (m, 2H), 1.10-1.03 (m, 2H).

### Example 102

### 2-(6-bromo-1-sulfanylidenespiro[3H-isoquinoline-4,1′-cyclopropane]-2-yl)-N-(5-fluoropyrimidin-2-yl)acetamide (102)

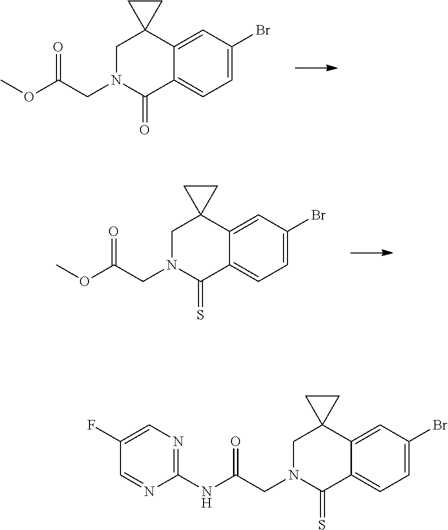 [SMILES: COC(=O)CN1CC2(CC2)c2cc(Br)ccc2C1=O ; COC(=O)CN1CC2(CC2)c2cc(Br)ccc2C1=S ; O=C(CN1CC2(CC2)c2cc(Br)ccc2C1=S)Nc1ncc(F)cn1]

Methyl 2-(6-bromo-1-sulfanylidenespiro[3H-isoquinoline-4,1′-cyclopropane]-2-yl)acetate: To a solution of methyl 2-(6-bromo-1-oxo-spiro[3H-isoquinoline-4,1′-cyclopropane]-2-yl)acetate (400 mg, 1.23 mmol) in toluene (10 mL) was added Lawesson's reagent (499 mg, 1.23 mmol). The reaction mixture was stirred at 90° C. for 6 h. The reaction mixture was concentrated under reduced pressure. The residue was purified by preparatory reverse-phase HPLC. LCMS: m/z=339.8, 341.7 [M+H]⁺.

2-(6-bromo-1-sulfanylidenespiro[3H-isoquinoline-4,1′-cyclopropane]-2-yl)-N-(5-fluoropyrimidin-2-yl)acetamide: To a solution of methyl 2-(6-bromo-1-sulfanylidenespiro[3H-isoquinoline-4,1′-cyclopropane]-2-yl)acetate (200 mg, 0.58 mmol) and 5-fluoropyrimidin-2-amine (199 mg, 1.76 mmol) in toluene (3.0 mL) and THF (1.0 mL) was added AlMe₃ (2 M in toluene, 0.88 mL). The reaction mixture was stirred at 100° C. for 3 h. The reaction mixture was poured into ice-cold water and extracted with EtOAc (3×5 mL). The combined organics were dried over anhydrous Na₂SO₄, filtered, and concentrated under reduced pressure. The residue was purified by preparatory reverse-phase HPLC. LCMS: m/z=420.9, 423.0 [M+H]⁺. ¹H NMR (400 MHz, CDCl₃): δ 9.19 (br s, 1H), 8.51-8.47 (m, 3H), 7.43 (dd, J=8.6, 1.9 Hz, 1H), 6.99 (d, J=2.0 Hz, 1H), 5.26 (br s, 2H), 3.67 (s, 2H), 1.18-1.12 (m, 2H), 1.11-1.06 (m, 2H).

### Example 103

### 2-(6-bromo-1-oxospiro[3H-isoquinoline-4,1′-cyclopropane]-2-yl)-N-(5-fluoropyrimidin-2-yl)ethanethioamide (103)

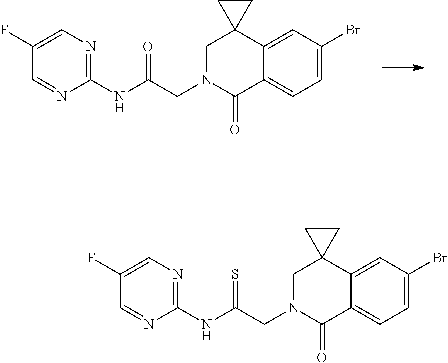 [SMILES: O=C(CN1CC2(CC2)c2cc(Br)ccc2C1=O)Nc1ncc(F)cn1 ; O=C1c2ccc(Br)cc2C2(CC2)CN1CC(=S)Nc1ncc(F)cn1]

To a solution of 2-(6-bromo-1-oxospiro[3H-isoquinoline-4,1′-cyclopropane]-2-yl)-N-(5-fluoropyrimidin-2-yl)acetamide (150 mg, 0.16 mmol) in toluene (5.0 mL) was added Lawesson's reagent (64 mg, 0.16 mmol). The reaction mixture was stirred at 100° C. for 1 h. The reaction mixture was poured into ice-cold water (10 mL) and extracted with EtOAc (4×5 mL). The combined organics were washed with brine (2×10 mL), dried over anhydrous Na₂SO₄, filtered, and concentrated under reduced pressure. The residue was purified by preparatory TLC. LCMS: m/z=421.0, 422.9 [M+H]⁺. ¹H NMR (400 MHz, DMSO-d₆): δ 12.43 (br s, 1H), 8.90 (s, 2H). 7.81 (d, J=8.0 Hz, 1H), 7.51 (dd, J=2.0, 8.4 Hz, 1H), 7.24 (d, J=2.0 Hz, 1H), 4.79 (br s, 2H), 3.55 (s, 2H), 1.18-1.12 (m, 2H), 1.08-1.01 (m, 2H).

### Example 104

### 2-(6-bromo-1-oxospiro[3H-isoquinoline-4,1′-cyclopropane]-2-yl)-N-(5-pyridin-2-ylpyrimidin-2-yl)acetamide (104)

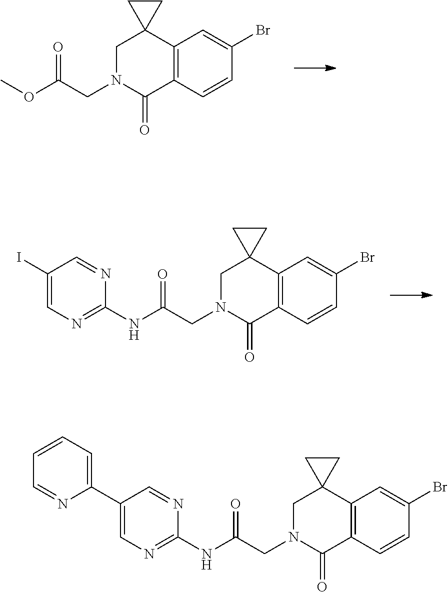 [SMILES: COC(=O)CN1CC2(CC2)c2cc(Br)ccc2C1=O ; O=C(CN1CC2(CC2)c2cc(Br)ccc2C1=O)Nc1ncc(I)cn1 ; O=C(CN1CC2(CC2)c2cc(Br)ccc2C1=O)Nc1ncc(-c2ccccn2)cn1]

2-(6-bromo-1-oxo-spiro[3H-isoquinoline-4,1′-cyclopropane]-2-yl)-N-(5-iodopyrimidin-2-yl)acetamide: To a solution of 5-iodopyrimidin-2-amine (409 mg, 1.85 mmol) and methyl 2-(6-bromo-1-oxo-spiro[3H-isoquinoline-4,1′-cyclopropane]-2-yl)acetate (300 mg, 0.93 mmol) in toluene (5 mL) was added AlMe₃ (2 M in toluene, 0.93 mL). The reaction mixture was stirred at 90° C. for 5 h. The reaction mixture was cooled to ambient temperature, poured into H₂O (10 mL), and extracted with EtOAc (3×10 mL). The combined organics were washed with brine (10 mL), dried over anhydrous Na₂SO₄, filtered, and concentrated under reduced pressure. The residue was purified by silica gel column chromatography. LCMS: m/z=513.0, 514.9 [M+H]⁺.

2-(6-bromo-1-oxospiro[3H-isoquinoline-4,1′-cyclopropane]-2-yl)-N-(5-pyridin-2-ylpyrimidin-2-yl)acetamide: A mixture of 2-(6-bromo-1-oxo-spiro[3H-isoquinoline-4,1′-cyclopropane]-2-yl)-N-(5-iodopyrimidin-2-yl)acetamide

(90 mg, 0.17 mmol), 2-(tributylstannyl)pyridine (65 mg, 0.17 mmol), Pd(PPh₃)₄ (10 mg, 0.09 mmol), LiCl (22 mg, 0.53 mmol) and CuI (100 mg, 0.53 mmol) in 1,4-dioxane (3 mL) was stirred at 50° C. for 16 h. The reaction mixture was poured into sat. aq. KF (5 mL) and extracted with EtOAc (3×5 mL). The combined organics were washed with brine (5 mL), dried over anhydrous Na₂SO₄, filtered, and concentrated under reduced pressure. The residue was purified by preparatory reverse-phase HPLC. LCMS: m/z=464.0, 466.0 [M+H]⁺.

¹H NMR (400 MHz, DMSO-d₆): δ 9.31 (s, 2H), 8.71 (d, J=5.6 Hz, 1H), 8.09 (d, J=8.6 Hz, 1H), 7.99-7.91 (m, 1H), 7.82 (d, J=8.6 Hz, 1H), 7.53 (d, J=10.0 Hz, 1H), 7.46-7.40 (m, 1H), 7.27 (s, 1H), 4.60 (s, 2H), 3.51 (s, 2H), 1.18-1.15 (m, 2H), 1.08-1.05 (m, 2H).

### Example 105

### 2-(6-bromo-5-fluoro-1-oxospiro[3H-isoquinoline-4,1′-cyclopropane]-2-yl)-N-(5-cyanopyrimidin-2-yl)acetamide (105)

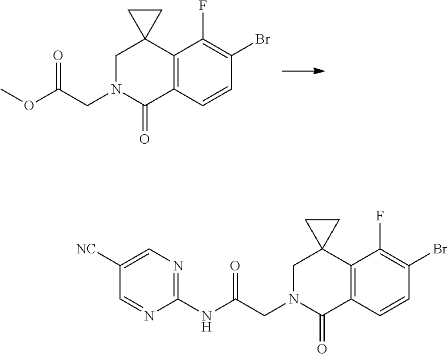 [SMILES: COC(=O)CN1CC2(CC2)c2c(ccc(Br)c2F)C1=O ; N#Cc1cnc(NC(=O)CN2CC3(CC3)c3c(ccc(Br)c3F)C2=O)nc1]

To a solution of 2-aminopyrimidine-5-carbonitrile (42 mg, 0.35 mmol) and methyl 2-(6-bromo-5-fluoro-1-oxo-spiro[3H-isoquinoline-4,1′-cyclopropane]-2-yl)acetate (100 mg, 0.29 mmol) in toluene (3.0 mL) was added DABAL-Me₃ (75 mg, 0.29 mmol). The reaction mixture was stirred for 12 h at 60° C. The reaction mixture was poured into ice-cold water (10 mL) and extracted with EtOAc (4×5 mL). The combined organics were washed with brine (10 mL), dried over anhydrous Na₂SO₄, filtered, and concentrated under reduced pressure. The residue was purified by reverse-phase preparative HPLC. LCMS: m/z=430.0, 432.0 [M+H]⁺. ¹H NMR (400 MHz, DMSO-d₆): δ 9.11 (s, 2H), 7.77-7.60 (m, 2H), 7.35 (br s, 1H), 4.58 (s, 2H), 3.46 (s, 2H), 1.53-1.42 (m, 2H), 1.13-1.06 (m, 2H).

### Example 106

### 2-(6-bromo-5-fluoro-1-oxospiro[3H-isoquinoline-4,1′-cyclopropane]-2-yl)-N-(5-cyano-3-fluoropyridin-2-yl)acetamide (106)

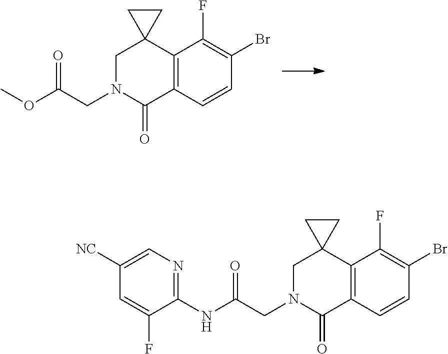 [SMILES: COC(=O)CN1CC2(CC2)c2c(ccc(Br)c2F)C1=O ; N#Cc1cnc(NC(=O)CN2CC3(CC3)c3c(ccc(Br)c3F)C2=O)c(F)c1]

To a solution of methyl 2-(6-bromo-1-oxo-spiro[3H-isoquinoline-4,1′-cyclopropane]-2-yl)acetate (50 mg, 0.15 mmol) in toluene (2.0 mL) was added 6-amino-5-fluoronicotinonitrile (20 mg, 0.15 mmol) and DABAL-Me₃ (38 mg, 0.15 mmol). The reaction mixture was stirred at 60° C. for 2 h. The reaction mixture was poured into ice-cold water (10 mL) and extracted with EtOAc (4×5 mL). The combined organics were washed with brine (10 mL), dried over anhydrous Na₂SO₄, filtered, and concentrated under reduced pressure. The residue was purified by reverse-phase preparative HPLC. LCMS: m/z=446.9, 448.9 [M+H]⁺. ¹H NMR (400 MHz, CDCl₃): δ 8.96 (br s, 1H), 8.50 (d, J=1.6 Hz, 1H), 7.87-7.85 (m, 1H), 7.71-7.68 (m, 1H), 7.54-7.50 (m, 1H), 4.57 (s, 2H), 3.48 (s, 2H), 1.66-1.63 (m, 2H), 1.05-1.02 (m, 2H).

### Example 107

### 2-(6-bromo-1-oxospiro[3H-isoquinoline-4,1′-cyclopropane]-2-yl)-N-(4-cyanopyrimidin-2-yl)acetamide (107)

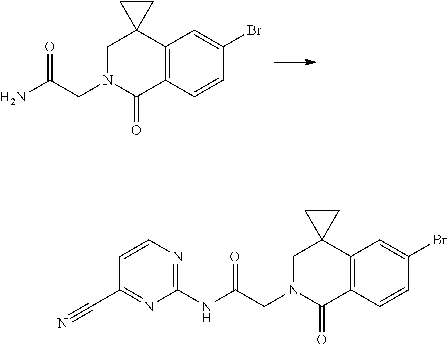 [SMILES: NC(=O)CN1CC2(CC2)c2cc(Br)ccc2C1=O ; N#Cc1ccnc(NC(=O)CN2CC3(CC3)c3cc(Br)ccc3C2=O)n1]

To a solution of 2-(6-bromo-1-oxo-spiro[3H-isoquinoline-4,1′-cyclopropane]-2-yl)acetamide (114 mg, 0.37 mmol) and 2-chloropyrimidine-4-carbonitrile (77 mg, 0.55 mmol) in THF (2.0 mL) was added Pd₂(dba)₃ (34 mg, 0.04 mmol), XPhos (18 mg, 0.04 mmol), and Cs₂CO₃ (240 mg, 0.07 mmol). The reaction mixture was stirred at 50° C. for 12 h. The reaction mixture was poured into ice-cold water (10 mL) and extracted with EtOAc (3×5 mL). The combined organics were washed with brine (10 mL), dried over anhydrous Na₂SO₄, filtered, and concentrated under reduced pressure. The residue was purified by reverse-phase preparative HPLC. LCMS: m/z=411.9, 414.0 [M+H]⁺. ¹H NMR (400 MHz, CDCl₃): δ 9.09 (br s, 1H), 8.84 (d, J=4.4 Hz, 1H), 8.02 (d, J=8.0 Hz, 1H), 7.47 (br d, J=7.2 Hz, 1H), 7.34 (d, J=4.8 Hz, 1H), 7.01 (s, 1H), 4.66 (s, 2H), 3.55 (s, 2H), 1.17-1.12 (m, 2H), 1.11-1.06 (m, 2H).

### Example 108

### 2-(5-fluoro-6-iodo-1-oxospiro[3H-isoquinoline-4,1′-cyclopropane]-2-yl)-N-(5-fluoropyrimidin-2-yl)acetamide (108)

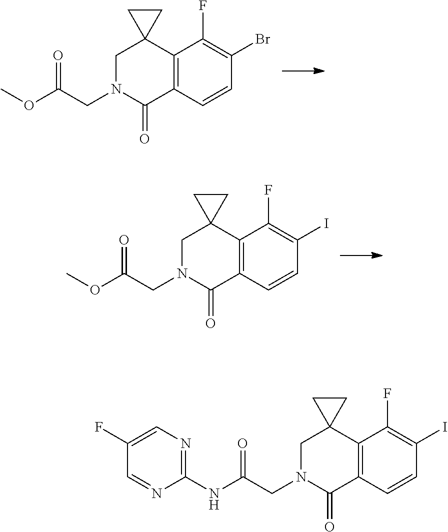 [SMILES: COC(=O)CN1CC2(CC2)c2c(ccc(Br)c2F)C1=O ; COC(=O)CN1CC2(CC2)c2c(ccc(I)c2F)C1=O ; O=C(CN1CC2(CC2)c2c(ccc(I)c2F)C1=O)Nc1ncc(F)cn1]

Methyl 2-(5-fluoro-6-iodo-1-oxospiro[3H-isoquinoline-4,1′-cyclopropane]-2-yl)acetate: To a solution of methyl 2-(6-bromo-5-fluoro-1-oxospiro[3H-isoquinoline-4,1′-cyclopropane]-2-yl)acetate (300 mg, 0.88 mmol), NaI (263 mg, 1.75 mmol) and N,N′-dimethyl-1,2-diaminocyclohexane (50 mg, 0.35 mmol) in toluene (5 mL) was added CuI (8 mg, 0.04 mmol). The mixture was stirred at 130° C. for 48 h. The reaction mixture was diluted with water (5 mL) and extracted with EtOAc (3×5 mL). The combined organics were washed with brine (5 mL), dried over anhydrous Na₂SO₄, filtered, and concentrated under reduced pressure. The residue was purified by silica gel column chromatography. ¹H NMR (400 MHz, CDCl₃): δ 7.71-7.69 (m, 2H), 4.31 (s, 2H), 3.77 (s, 3H), 3.37 (s, 2H), 1.65-1.58 (m, 2H), 1.04-0.97 (m, 2H).

2-(5-fluoro-6-iodo-1-oxospiro[3H-isoquinoline-4,1′-cyclopropane]-2-yl)-N-(5-fluoropyrimidin-2-yl)acetamide: To a solution of methyl 2-(5-fluoro-6-iodo-1-oxospiro[3H-isoquinoline-4,1′-cyclopropane]-2-yl)acetate (0.14 g, 0.36 mmol) and 5-fluoropyrimidin-2-amine (90 mg, 0.79 mmol) in DCE (5 mL) was added DABAL-Me₃ (203 mg, 0.79 mmol). The mixture was stirred at 60° C. for 16 h. The reaction mixture was diluted with water (15 mL) and filtered. The filtrate was extracted with EtOAc (3×10 mL). The combined organic layers were dried over anhydrous Na₂SO₄, filtered, and concentrated under reduced pressure. The residue was purified by reverse-phase preparative HPLC. LCMS: m/z=471.1 [M+H]⁺. ¹H NMR (400 MHz, CDCl₃): δ 8.86 (br s, 1H), 8.50 (s, 2H), 7.74-7.71 (m, 2H), 4.58 (s, 2H), 3.46 (s, 2H), 1.65-1.59 (m, 2H), 1.06-0.99 (m, 2H).

### Example 109

### 2-[(2′s,4r)-6-bromo-2′-fluoro-1-oxospiro[3H-isoquinoline-4,1′-cyclopropane]-2-yl]-N-(pyrimidin-2-yl)acetamide (109)

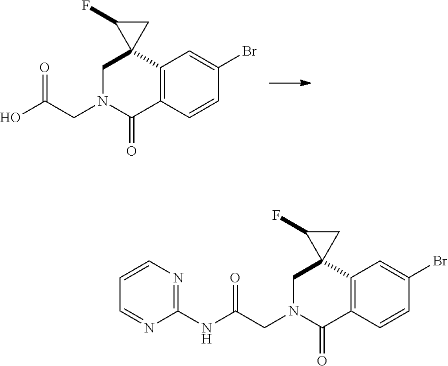

To a solution of 2-[(2′s,4r)-6-bromo-2′-fluoro-1-oxospiro[3H-isoquinoline-4,1′-cyclopropane]-2-yl]acetic acid (50 mg, 0.15 mmol, Int. 34) in pyridine (1.0 mL) were added pyrimidin-2-amine (36 mg, 0.38 mmol) and EDCI (58 mg, 0.31 mmol). The reaction mixture was stirred at 20° C. for 6 h. The reaction mixture was diluted with water (2 mL) and extracted with EtOAc (3×2 mL). The combined organics were washed with brine (2 mL), dried over anhydrous Na₂SO₄, filtered, and concentrated under reduced pressure. The residue was purified by preparatory reverse-phase HPLC. LCMS: m/z=405.0, 407.0 [M+H]⁺. ¹H NMR (400 MHz, DMSO-d₆): δ 10.81 (s, 1H), 8.67 (d, J=4.8 Hz, 2H), 7.83 (d, J=8.4 Hz, 1H), 7.59 (dd, J=1.6, 8.4 Hz, 1H), 7.25 (d, J=1.6 Hz, 1H), 7.19 (t, J=4.4 Hz. 1H), 5.19-4.98 (m, 1H), 4.80 (br d, J=17.2 Hz, 1H), 4.35 (br d, J=17.2 Hz, 1H), 3.93 (dd, J=1.6, 13.2 Hz, 1H), 3.55 (d, J=12.8 Hz, 1H), 1.81-1.72 (m, 1H), 1.56-1.45 (m, 1H).

Example 109 has been identified as a single enantiomer, 2-[(2′R,4S)-6-bromo-2′-fluoro-1-oxospiro[3H-isoquinoline-4,1′-cyclopropane]-2-yl]-N-pyrimidin-2-ylacetamide, as shown in Table 1A.

### Example 110

### 2-[(2′s,4r)-6-bromo-2′-fluoro-1-oxospiro[3H-isoquinoline-4,1′-cyclopropane]-2-yl]-N-(5-chloropyrimidin-2-yl)acetamide (110)

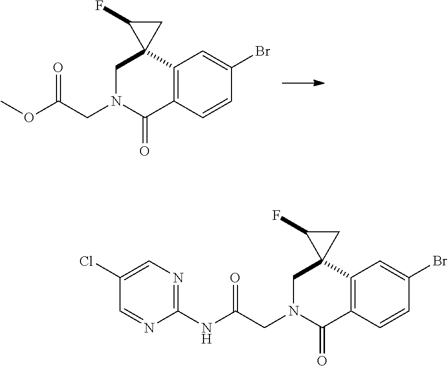

To a solution of methyl 2-[(2′s,4r)-6-bromo-2′-fluoro-1-oxospiro[3H-isoquinoline-4,1′-cyclopropane]-2-yl]acetate (60 mg, 0.18 mmol, Int. 33) and 5-chloropyrimidin-2-amine (68 mg, 0.53 mmol) in DCE (1 mL) was added AlMe₃ (0.52 mL. 1 M in n-heptane). The reaction mixture was stirred at 90° C. for 2 h. The reaction mixture was diluted with sat. aq. NH₄Cl (5 mL) and extracted with EtOAc (3×5 mL). The combined organics were washed with brine (5 mL), dried over anhydrous Na₂SO₄, filtered, and concentrated under reduced pressure. The residue was purified by preparatory reverse-phase HPLC. LCMS: m/z=438.9, 440.9, 442.9 [M+H]⁺. ¹H NMR (400 MHz, CDCl₃): δ 8.78 (s, 2H), 7.83 (d, J=8.0 Hz, 1H), 7.59 (dd, J=2.0, 8.0 Hz, 1H), 7.25 (d, J=2.0 Hz, 1H), 6.17-5.95 (s, 1H), 5.21-4.96 (m, 1H), 4.76 (d, J=16.0 Hz, 1H), 4.32 (d, J=16.0 Hz, 1H), 3.93 (d, J=12.0 Hz, 1H), 3.55 (d, J=12.0 Hz, 1H). 1.79-1.73 (m, 1H), 1.56-1.45 (m, 1H).

Example 110 has been identified as a single enantiomer, 2-[(2′R,4S)-6-bromo-2′-fluoro-1-oxospiro[3H-isoquinoline-4,1′-cyclopropane]-2-yl]-N-(5-chloropyrimidin-2-yl)acetamide, as shown in Table 1A.

### Example 111

### 2-(6-bromo-1-oxospiro[3H-isoquinoline-4,1′-cyclopropane]-2-yl)-N-[3-(difluoromethyl)cyclobutyl]acetamide (111)

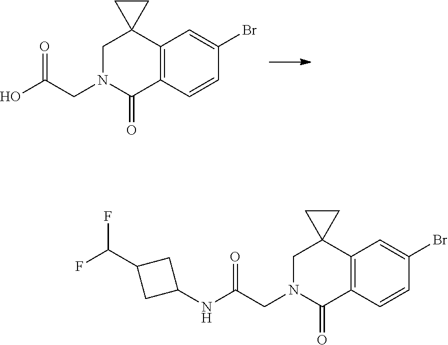 [SMILES: O=C(O)CN1CC2(CC2)c2cc(Br)ccc2C1=O ; O=C(CN1CC2(CC2)c2cc(Br)ccc2C1=O)NC1CC(C(F)F)C1]

To a solution of 2-(6-bromo-1-oxo-spiro[3H-isoquinoline-4,1′-cyclopropane]-2-yl)acetic acid (20 mg, 0.06 mmol) in DMF (1.0 mL) were added 3-(difluoromethyl)cyclobutanamine HCl salt (14 mg, 0.09 mmol), DIPEA (25 mg, 0.19 mmol), and T3P (53 mg, 0.08 mmol, 50% in DMF). The reaction mixture was stirred at 25° C. for 1 h. The reaction mixture was directly purified directly by reverse-phase preparative HPLC. LCMS: m/z=413.3, 415.3 [M+H]⁺. ¹H NMR (400 MHz, DMSO-d₆): δ 8.37-8.28 (m, 1H), 7.81 (dd, J=8.3, 1.3 Hz, 1H), 7.52 (ddd, J=8.3, 1.9, 0.6 Hz, 1H), 7.24 (d, J=1.8 Hz, 1H), 6.35-5.86 (m, 1H), 4.32-4.14 (m, 1H), 4.15 (dt, J=1.0, 0.5 Hz, 2H), 3.47-3.37 (m, 2H), 2.43-2.08 (m, 5H), 1.15-1.12 (m, 2H), 1.03 (dt, J=5.2, 2.8 Hz, 2H).

The following compound was, or can be, made via similar procedures as those described above (coupling reagents employed include T3P, HATU, and EDCI):

| Ex. | Name | NMR | LCMS |
|---|---|---|---|
| 112 | 2-(6-bromo-1-oxospiro[3H- | ¹H NMR (400 MHZ, DMSO-d₆): δ 8.09 | m/z = |
| | isoquinoline-4,1′-cyclopropane]-2- | (t, J = 6.1 Hz, 1H), 7.81 (d, J = 8.3 Hz, | 441.3, |
| | yl)-N-[[1-(2,2- | 1H), 7.52 (dd, J = 8.3, 1.9 Hz, 1H), 7.24 | 443.3 |
| | difluoroethyl)cyclobutyl]methyl] | (d, J = 1.9 Hz, 1H), 6.18 (tt, J = 56.3, 5.0 | [M + H]⁺ |
| | acetamide | Hz, 1H), 4.16 (s, 2H), 3.45 (s, 2H), 3.27 | |
| | | (d, J = 6.2 Hz, 2H), 1.95 (td, J = 17.9, | |
| | | 4.9 Hz, 2H), 1.86-1.79 (m, 6H), 1.15 (t, J = | |
| | | 3.6 Hz, 2H), 1.05-1.02 (m, 2H) | |
| 113 | 2-(6-bromo-1-oxospiro[3H- | ¹H NMR (400 MHZ, DMSO-d₆): δ 9.64 | m/z = |
| | isoquinoline-4,1′-cyclopropane]-2- | (s, 1H), 7.95-7.92 (m, 1H), 7.83 (d, J = | 451.3, |
| | yl)-N-[2- | 8.3 Hz, 1H), 7.53 (d, J = 8.3, 1H), 7.27- | 453.3 |
| | (difluoromethoxy)phenyl]acetamide | 7.18 (m, 4H), 7.16 (t, J = 73.7 Hz, 1H), | [M + H]⁺ |
| | | 4.39 (s, 2H), 3.51 (s, 2H), 1.18-1.15 (m, | |
| | | 2H), 1.08-1.05 (m, 2H) | |
| 114 | 2-(6-bromo-1-oxospiro[3H- | | m/z = |
| | isoquinoline-4,1′-cyclopropane]-2- | | 400.0, |
| | yl)-N-(6-methylpyridin-2- | | 402.0 |
| | yl)acetamide | | [M + H]⁺ |
| 115 | 2-(6-bromo-1-oxospiro[3H- | | m/z = |
| | isoquinoline-4,1′-cyclopropane]-2- | | 404.0, |
| | yl)-N-(3,4-dimethyl-1,2-oxazol-5- | | 406.0 |
| | yl)acetamide | | [M + H]⁺ |
| 116 | 2-(6-bromo-1-oxospiro[3H- | | m/z = |
| | isoquinoline-4,1′-cyclopropane]-2- | | 425.1, |
| | yl)-N-imidazo[1,2-a]pyridin-5- | | 427.1 |
| | ylacetamide | | [M + H]⁺ |
| 117 | 2-(6-bromo-1-oxospiro[3H- | | m/z = |
| | isoquinoline-4,1′-cyclopropane]-2- | | 379.0, |
| | yl)-N-(oxetan-3-ylmethyl)acetamide | | 381.0 |
| | | | [M + H]⁺ |
| 118 | 2-(6-bromo-1-oxospiro[3H- | | m/z = |
| | isoquinoline-4,1′-cyclopropane]-2- | | 412.9, |
| | yl)-N-(1,1-dioxothietan-3- | | 414.9 |
| | yl)acetamide | | [M + H]⁺ |
| 119 | 2-(6-bromo-1-oxospiro[3H- | | m/z = |
| | isoquinoline-4,1′-cyclopropane]-2- | | 390.0, |
| | yl)-N-(1,3-oxazol-2- | | 392.0 |
| | ylmethyl)acetamide | | [M + H]⁺ |
| 120 | 2-(6-bromo-1-oxospiro[3H- | | m/z = |
| | isoquinoline-4,1′-cyclopropane]-2- | | 403.0, |
| | yl)-N-[(1-methylimidazol-2- | | 405.0 |
| | yl)methyl]acetamide | | [M + H]⁺ |
| 121 | 2-(6-bromo-1-oxospiro[3H- | | m/z = |
| | isoquinoline-4,1′-cyclopropane]-2- | | 403.0, |
| | yl)-N-(2-imidazol-1- | | 405.0 |
| | ylethyl)acetamide | | [M + H]⁺ |
| 122 | 2-(6-bromo-1-oxospiro[3H- | | m/z = |
| | isoquinoline-4,1′-cyclopropane]-2- | | 400.0, |
| | yl)-N-(pyridin-4-ylmethyl)acetamide | | 402.0 |
| | | | [M + H]⁺ |
| 123 | 2-(6-bromo-1-oxospiro[3H- | | m/z = |
| | isoquinoline-4,1′-cyclopropane]-2- | | 417.0, |
| | yl)-N-methyl-N-[(1-methylpyrazol-4- | | 419.0 |
| | yl)methyl]acetamide | | [M + H]⁺ |
| 124 | 2-(6-bromo-1-oxospiro[3H- | | m/z = |
| | isoquinoline-4,1′-cyclopropane]-2- | | 403.0, |
| | yl)-N-[(2-methylpyrazol-3- | | 405.0 |
| | yl)methyl]acetamide | | [M + H]⁺ |
| 125 | 6-bromo-2-[2-(5,7- | | m/z = |
| | dihydropyrrolo[3,4-b]pyridin-6-yl)-2- | | 412.0, |
| | oxoethyl]spiro[3H-isoquinoline-4,1′- | | 414.0 |
| | cyclopropane]-1-one | | [M + H]⁺ |
| 126 | 2-(6-bromo-1-oxospiro[3H- | | m/z = |
| | isoquinoline-4,1′-cyclopropane]-2- | | 429.0, |
| | yl)-N-(5-cyclopropyl-2- | | 431.0 |
| | methylpyrazol-3-yl)acetamide | | [M + H]⁺ |
| 127 | 2-(6-bromo-1-oxospiro[3H- | | m/z = |
| | isoquinoline-4,1′-cyclopropane]-2- | | 385.0, |
| | yl)-N-phenylacetamide | | 387.0 |
| | | | [M + H]⁺ |
| 128 | 2-(6-bromo-1-oxospiro[3H- | | m/z = |
| | isoquinoline-4,1′-cyclopropane]-2- | | 422.2, |
| | yl)-N-(2-morpholin-4- | | 424.1 |
| | ylethyl)acetamide | | [M + H]⁺ |
| 129 | 2-(6-bromo-1-oxospiro[3H- | | m/z = |
| | isoquinoline-4,1′-cyclopropane]-2- | | 425.0, |
| | yl)-N-(2,3-dihydro-1H-inden-2- | | 427.0 |
| | yl)acetamide | | [M + H]⁺ |
| 130 | 2-(6-bromo-1-oxospiro[3H- | | m/z = |
| | isoquinoline-4,1′-cyclopropane]-2- | | 436.0, |
| | yl)-N-quinolin-6-ylacetamide | | 438.0 |
| | | | [M + H]⁺ |
| 131 | 2-(6-bromo-1-oxospiro[3H- | | m/z = |
| | isoquinoline-4,1′-cyclopropane]-2- | | 436.0, |
| | yl)-N-quinolin-3-ylacetamide | | 438.0 |
| | | | [M + H]⁺ |
| 132 | 2-(6-bromo-1-oxospiro[3H- | | m/z = |
| | isoquinoline-4,1′-cyclopropane]-2- | | 447.0, |
| | yl)-N-[2-(4-fluorophenyl)-2- | | 449.0 |
| | hydroxyethyl]acetamide | | [M + H]⁺ |
| 133 | 2-(6-bromo-1-oxospiro[3H- | | m/z = |
| | isoquinoline-4,1′-cyclopropane]-2- | | 405.0, |
| | yl)-N-(3,3,3- | | 407.0 |
| | trifluoropropyl)acetamide | | [M + H]⁺ |
| 134 | 2-(6-bromo-1-oxospiro[3H- | | m/z = |
| | isoquinoline-4,1′-cyclopropane]-2- | | 376.0, |
| | yl)-N-(2-cyanopropan-2- | | 378.0 |
| | yl)acetamide | | [M + H]⁺ |
| 135 | 2-(6-bromo-1-oxospiro[3H- | | m/z = |
| | isoquinoline-4,1′-cyclopropane]-2- | | 427.0, |
| | yl)-N-(2,3-dihydro-1-benzofuran-5- | | 429.0 |
| | yl)acetamide | | [M + H]⁺ |
| 136 | 2-(6-bromo-1-oxospiro[3H- | | m/z = |
| | isoquinoline-4,1′-cyclopropane]-2- | | 436.1, |
| | yl)-N-isoquinolin-6-ylacetamide | | 438.1 |
| | | | [M + H]⁺ |
| 137 | 2-(6-bromo-1-oxospiro[3H- | | |
| | isoquinoline-4,1′-cyclopropane]-2- | | m/z = |
| | yl)-N-[2- | | 429.9, |
| | (methanesulfonamido)ethyl] | | 431.9 |
| | acetamide | | [M + H]⁺ |
| 138 | 2-(6-bromo-1-oxospiro[3H- | | m/z = |
| | isoquinoline-4,1′-cyclopropane]-2- | | 433.0, |
| | yl)-N-[1-(2-methoxyethyl)pyrazol-4- | | 435.0 |
| | yl]acetamide | | [M + H]⁺ |
| 139 | 2-(6-bromo-1-oxospiro[3H- | | m/z = |
| | isoquinoline-4,1′-cyclopropane]-2- | | 415.0, |
| | yl)-N-(2- | | 416.9 |
| | methylsulfonylethyl)acetamide | | [M + H]⁺ |
| 140 | N-(1,3-benzothiazol-6-yl)-2-(6- | | m/z = |
| | bromo-1-oxospiro[3H-isoquinoline- | | 441.9, |
| | 4,1′-cyclopropane]-2-yl)acetamide | | 443.9 |
| | | | [M + H]⁺ |
| 141 | 2-(6-bromo-1-oxospiro[3H- | | m/z = |
| | isoquinoline-4,1′-cyclopropane]-2- | | 439.0, |
| | yl)-N-(1-methylindazol-5- | | 441.0 |
| | yl)acetamide | | [M + H]⁺ |
| 142 | 2-(6-bromo-1-oxospiro[3H- | | m/z = |
| | isoquinoline-4,1′-cyclopropane]-2- | | 482.0, |
| | yl)-N-[3-(2-methyl-1,3-thiazol-4- | | 484.0 |
| | yl)phenyl]acetamide | | [M + H]⁺ |
| 143 | 2-(6-bromo-1-oxospiro[3H- | | m/z = |
| | isoquinoline-4,1′-cyclopropane]-2- | | 455.9, |
| | yl)-N-(2-methyl-1,3-benzothiazol-6- | | 458.0 |
| | yl)acetamide | | [M + H]⁺ |
| 144 | 2-(6-bromo-1-oxospiro[3H- | | m/z = |
| | isoquinoline-4,1′-cyclopropane]-2- | | 403.0, |
| | yl)-N-(2,5-dimethylpyrazol-3- | | 405.0 |
| | yl)acetamide | | [M + H]⁺ |
| 145 | N-(1H-benzimidazol-2-yl)-2-(6- | | m/z = |
| | bromo-1-oxospiro[3H-isoquinoline- | | 425.0, |
| | 4,1′-cyclopropane]-2-yl)acetamide | | 427.0 |
| | | | [M + H]⁺ |
| 146 | N-(1,3-benzothiazol-5-yl)-2-(6- | | m/z = |
| | bromo-1-oxospiro[3H-isoquinoline- | | 442.0, |
| | 4,1′-cyclopropane]-2-yl)acetamide | | 444.0 |
| | | | [M + H]⁺ |
| 147 | 2-(6-bromo-1-oxospiro[3H- | | m/z = |
| | isoquinoline-4,1′-cyclopropane]-2- | | 439.0, |
| | yl)-N-(1-methylbenzimidazol-5- | | 441.0 |
| | yl)acetamide | | [M + H]⁺ |
| 148 | 2-(6-bromo-1-oxospiro[3H- | | m/z = |
| | isoquinoline-4,1′-cyclopropane]-2- | | 436.0, |
| | yl)-N-isoquinolin-8-ylacetamide | | 438.0 |
| | | | [M + H]⁺ |
| 149 | 2-(6-bromo-1-oxospiro[3H- | | m/z = |
| | isoquinoline-4,1′-cyclopropane]-2- | | 465.0, |
| | yl)-N-(5-methyl-2-phenylpyrazol-3- | | 467.0 |
| | yl)acetamide | | [M + H]⁺ |
| 150 | 2-(6-bromo-1-oxospiro[3H- | | m/z = |
| | isoquinoline-4,1′-cyclopropane]-2- | | 414.0, |
| | yl)-N-(4,6-dimethylpyridin-2- | | 416.0 |
| | yl)acetamide | | [M + H]⁺ |
| 151 | 2-(6-bromo-1-oxospiro[3H- | | m/z = |
| | isoquinoline-4,1′-cyclopropane]-2- | | 468.0, |
| | yl)-N-[2-(trifluoromethyl)pyridin-3- | | 470.0 |
| | yl]methyl]acetamide | | [M + H]⁺ |
| 152 | 2-(6-bromo-1-oxospiro[3H- | | m/z = |
| | isoquinoline-4,1′-cyclopropane]-2- | | 451.0, |
| | yl)-N-(2-phenylpyrazol-3- | | 453.0 |
| | yl)acetamide | | [M + H]⁺ |
| 153 | 2-(6-bromo-1-oxospiro[3H- | | m/z = |
| | isoquinoline-4,1′-cyclopropane]-2- | | 436.0, |
| | yl)-N-isoquinolin-4-ylacetamide | | 438.0 |
| | | | [M + H]⁺ |
| 154 | 2-(6-bromo-1-oxospiro[3H- | | m/z = |
| | isoquinoline-4,1′-cyclopropane]-2- | | 416.0, |
| | yl)-N-(5-methyl-2-oxo-1H-pyridin-3- | | 418.0 |
| | yl)acetamide | | [M + H]⁺ |
| 155 | 2-(6-bromo-1-oxospiro[3H- | | m/z = |
| | isoquinoline-4,1′-cyclopropane]-2- | | 425.0, |
| | yl)-N-imidazo[1,2-a]pyridin-8- | | 427.0 |
| | ylacetamide | | [M + H]⁺ |
| 156 | 2-(6-bromo-1-oxospiro[3H- | | m/z = |
| | isoquinoline-4,1′-cyclopropane]-2- | | 425.0, |
| | yl)-N-(1H-pyrrolo[2,3-b]pyridin-5- | | 427.0 |
| | yl)acetamide | | [M + H]⁺ |
| 157 | 2-(6-bromo-1-oxospiro[3H- | | m/z = |
| | isoquinoline-4,1′-cyclopropane]-2- | | 464.9, |
| | yl)-N-(2,2-difluoro-1,3-benzodioxol- | | 466.9 |
| | 5-yl)acetamide | | [M + H]⁺ |
| 158 | 6-bromo-2-[2-(2,3- | | m/z = |
| | dihydropyrrolo[2,3-b]pyridin-1-yl)-2- | | 412.0, |
| | oxoethyl]spiro[3H-isoquinoline-4,1′- | | 414.0 |
| | cyclopropane]-1-one | | [M + H]⁺ |
| 159 | 2-(6-bromo-1-oxospiro[3H- | | m/z = |
| | isoquinoline-4,1′-cyclopropane]-2- | | 425.9, |
| | yl)-N-([1,2,4]triazolo[4,3-a]pyridin- | | 428.0 |
| | 6-yl)acetamide | | [M + H]⁺ |
| 160 | 2-(6-bromo-1-oxospiro[3H- | | m/z = |
| | isoquinoline-4,1′-cyclopropane]-2- | | 379.0, |
| | yl)-N-[1- | | 381.0 |
| | (hydroxymethyl)cyclopropyl] | | [M + H]⁺ |
| | acetamide | | |
| 161 | 2-(6-bromo-1-oxospiro[3H- | | m/z = |
| | isoquinoline-4,1′-cyclopropane]-2- | | 425.0, |
| | yl)-N-(1- | | 427.0 |
| | phenylcyclopropyl)acetamide | | [M + H]⁺ |
| 162 | 2-(6-bromo-1-oxospiro[3H- | | m/z = |
| | isoquinoline-4,1′-cyclopropane]-2- | | 436.0, |
| | yl)-N-quinolin-5-ylacetamide | | 438.0 |
| | | | [M + H]⁺ |
| 163 | 2-(6-bromo-1-oxospiro[3H- | | m/z = |
| | isoquinoline-4,1′-cyclopropane]-2- | | 410.0, |
| | yl)-N-(5-fluoro-1,3-thiazol-2- | | 412.0 |
| | yl)acetamide | | [M + H]⁺ |
| 164 | 2-(6-bromo-1-oxospiro[3H- | | m/z = |
| | isoquinoline-4,1′-cyclopropane]-2- | | 377.0, |
| | yl)-N-(cyclobutylmethyl)acetamide | | 379.0 |
| | | | [M + H]⁺ |
| 165 | 2-(6-bromo-1-oxospiro[3H- | | m/z = |
| | isoquinoline-4,1′-cyclopropane]-2- | | 504.1, |
| | yl)-N-[1-(6-chloropyridazin-3- | | 506.1 |
| | yl)piperidin-4-yl]acetamide | | [M + H]⁺ |
| 166 | 2-(6-bromo-1-oxospiro[3H- | | m/z = |
| | isoquinoline-4,1′-cyclopropane]-2- | | 425.1, |
| | yl)-N-(1H-indazol-3-yl)acetamide | | 427.1 |
| | | | [M + H]⁺ |
| 167 | N-(1H-benzotriazol-5-yl)-2-(6- | | m/z = |
| | bromo-1-oxospiro[3H-isoquinoline- | | 426.2, |
| | 4,1′-cyclopropane]-2-yl)acetamide | | 428.2 |
| | | | [M + H]⁺ |
| 168 | 2-(6-bromo-1-oxospiro[3H- | | m/z = |
| | isoquinoline-4,1′-cyclopropane]-2- | | 415.0, |
| | yl)-N-(3-cyclopropyl-1H-pyrazol-5- | | 417.0 |
| | yl)acetamide | | [M + H]⁺ |
| 169 | N-(3H-benzimidazol-5-yl)-2-(6- | | m/z = |
| | bromo-1-oxospiro[3H-isoquinoline- | | 425.1, |
| | 4,1′-cyclopropane]-2-yl)acetamide | | 427.1 |
| | | | [M + H]⁺ |
| 170 | 2-(6-bromo-1-oxospiro[3H- | | m/z = |
| | isoquinoline-4,1′-cyclopropane]-2- | | 425.1, |
| | yl)-N-(1H-indazol-5-yl)acetamide | | 427.1 |
| | | | [M + H]⁺ |
| 171 | 2-(6-bromo-1-oxospiro[3H- | | m/z = |
| | isoquinoline-4,1′-cyclopropane]-2- | | 401.1, |
| | yl)-N-(6-methylpyrazin-2- | | 403.1 |
| | yl)acetamide | | [M + H]⁺ |
| 172 | 2-(6-bromo-1-oxospiro[3H- | | m/z = |
| | isoquinoline-4,1′-cyclopropane]-2- | | 453.1, |
| | yl)-N-[4-(1H-tetrazol-5- | | 455.1 |
| | yl)phenyl]acetamide | | [M + H]⁺ |
| 173 | N-(1,3-benzoxazol-5-yl)- | | m/z = |
| | 2-(6-bromo- | | 426.1, |
| | 1-oxospiro[3H-isoquinoline-4,1′- | | 428.1 |
| | cyclopropane]-2-yl)acetamide | | [M + H]⁺ |
| 174 | 2-(6-bromo-1-oxospiro[3H- | | m/z = |
| | isoquinoline-4,1′-cyclopropane]-2- | | 421.1, |
| | yl)-N-[1-(2-hydroxy-2- | | 423.1 |
| | methylpropyl)cyclopropyl]acetamide | | [M + H]⁺ |

### Example 175

### 2-(6-bromo-1-oxospiro[3H-isoquinoline-4,1′-cyclopropane]-2-yl)-N-pyrazolo[1,5-a]pyrimidin-5-ylacetamide (175)

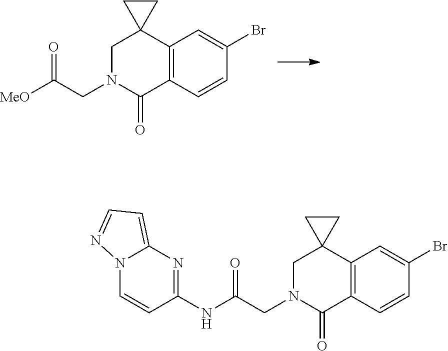 [SMILES: COC(=O)CN1CC2(CC2)c2cc(Br)ccc2C1=O ; O=C(CN1CC2(CC2)c2cc(Br)ccc2C1=O)Nc1ccn2nccc2n1]

To a solution of pyrazolo[1,5-a]pyrimidin-5-amine (37 mg, 0.28 mmol), methyl 2-(6-bromo-1-oxo-spiro[3H-isoquinoline-4,1′-cyclopropane]-2-yl)acetate (45 mg, 0.14 mmol) in DCE (1.0 mL) was added AlMe₃ (0.20 mL, 2 M in toluene). The reaction mixture was stirred at 25° C. for 1 h then 90° C. for 30 min. The reaction mixture was diluted with water (0.5 mL) and extracted with EtOAc (2×5 mL). The combined organics were dried over anhydrous Na₂SO₄, filtered, and concentrated under reduced pressure. The residue was purified by reverse-phase preparative HPLC. LCMS: m/z=426.3, 428.3 [M+H]⁺. ¹H NMR (400 MHz, DMSO-d₆): δ 11.17 (s, 1H), 9.02 (dd, J=7.7, 0.9 Hz, 1H), 8.12 (d, J=2.3 Hz, 1H), 7.82 (d, J=8.3 Hz, 1H), 7.71-7.69 (in, 1H), 7.53 (dd, J=8.3, 1.9 Hz, 1H), 7.27 (d, J=1.9 Hz, 1H), 6.44 (dd, J=2.3, 0.9 Hz, 1H), 4.44 (s, 2H), 3.53 (s, 2H), 1.19-1.17 (m, 2H), 1.08-1.05 (m, 2H).

The following compound was, or can be, made via similar procedures as those described above:

| Ex. | Name | NMR | LCMS |
|---|---|---|---|
| 176 | 2-(6-bromo-1-oxospiro[3H- | ¹H NMR (400 MHZ, DMSO-d₆): δ 10.98 (s, | m/z = |
| | isoquinoline-4,1′- | 1H), 9.24 (t, J = 0.9 Hz, 1H), 8.91 (dd, J = | 437.4, |
| | cyclopropane]-2-yl)-N- | 4.1, 1.6 Hz, 1H), 8.50 (s, 1H), 8.40-8.37 (m, | 439.3 |
| | (1,7-naphthyridin-6- | 1H), 7.83 (d, J = 8.3 Hz, 1H), 7.70 (dd, J = | [M + H]⁺ |
| | yl)acetamide | 8.4, 4.1 Hz, 1H), 7.54 (dd, J = 8.3, 1.9 Hz, | |
| | | 1H), 7.28 (d, J = 1.9 Hz, 1H), 4.46 (s, 2H), | |
| | | 3.56 (s, 2H), 1.20-1.18 (m, 2H), 1.10-1.06 (m, | |
| | | 2H) | |
| 177 | 2-(6-cyano-1-oxospiro[3H- | ¹H NMR (400 MHZ, acetone-d₆): δ 9.80 (s, | m/z = |
| | isoquinoline-4,1′- | 1H), 8.64 (s, 2H), 8.18 (d, J = 8.0 Hz, 1H), | 352.3 |
| | cyclopropane]-2-yl)-N-(5- | 7.75-7.72 (m, 1H), 7.50 (dd, J = 4.5, 0.9 Hz, | [M + H]⁺ |
| | fluoropyrimidin-2- | 1H), 4.80 (s, 2H), 3.67 (s, 2H), 1.23-1.19 (m, | |
| | yl)acetamide | 4H) | |
| 178 | 2-(6-bromo-4,4-dimethyl-1- | ¹H NMR (400 MHZ, MeOD): δ 8.60 (d, J = | m/z = |
| | oxo-3H-isoquinolin-2-yl)- | 0.6 Hz, 2H), 7.89 (d, J = 8.3 Hz, 1H), 7.63 (d, | 407.2, |
| | N-(5-fluoropyrimidin-2- | J = 1.9 Hz, 1H), 7.55 (dd, J = 8.3, 1.9 Hz, | 409.2 |
| | yl)acetamide | 1H), 4.62 (s, 2H), 3.58 (s, 2H), 1.42 (s, 6H) | [M + H]⁺ |
| 179 | 2-(6-bromo-1-oxospiro[3H- | ¹H NMR (400 MHZ, DMSO-d₆): δ 10.78 (s, | m/z = |
| | isoquinoline-4,1′- | 1H), 9.26 (d, J = 1.4 Hz, 1H), 8.95 (dd, J = | 426.3, |
| | cyclopropane]-2-yl)-N- | 1.5, 0.7 Hz, 1H), 8.22 (t, J = 0.8 Hz, 1H), | 428.3 |
| | imidazo[1,2-a]pyrazin-6- | 7.83-7.79 (m, 2H), 7.53 (dd, J = 8.3, 1.9 Hz, | [M + H]⁺ |
| | ylacetamide | 1H), 7.28 (d, J = 1.9 Hz, 1H), 4.42 (s, 2H), | |
| | | 3.54 (s, 2H), 1.20-1.17 (m, 2H), 1.09-1.06 (m, | |
| | | 2H) | |
| 180 | 2-(6-bromo-1-oxospiro[3H- | ¹H NMR (400 MHZ, DMSO-d₆): δ 11.09 (s, | m/z = |
| | isoquinoline-4,1′- | 1H), 8.15-8.10 (m, 2H), 7.88-7.81 (m, 2H), | 426.3, |
| | cyclopropane]-2-yl)-N- | 7.72 (d, J = 1.2 Hz, 1H), 7.53 (dd, J = 8.3, 1.9 | 428.3 |
| | imidazo[1,2-b]pyridazin-6- | Hz, 1H), 7.27 (d, J = 1.9 Hz, 1H), 4.43 (s, | [M + H]⁺ |
| | ylacetamide | 2H), 3.54 (s, 2H), 1.19-1.16 (m, 2H), 1.08- | |
| | | 1.05 (m, 2H) | |
| 181 | 2-(6-bromo-1-oxospiro[3H- | ¹H NMR (400 MHz, DMSO-d₆): δ 11.05 (s, | m/z = |
| | isoquinoline-4,1′- | 1H), 8.85 (d, J = 6.7 Hz, 1H), 7.82 (d, J = 8.3 | 426.3, |
| | cyclopropane]-2-yl)-N- | Hz, 1H), 7.72-7.62 (m, 2H), 7.53 (dd, J = 8.3, | 428.2 |
| | ([1,2,4]triazolo[1,5- | 1.9 Hz, 1H), 7.27 (d, J = 1.8 Hz, 1H), 7.14 (td, | [M + H]⁺ |
| | alpyridin-2-yl)acetamide | J = 6.8, 1.5 Hz, 1H), 4.52-4.48 (br s, 2H), | |
| | | 3.52 (s, 2H), 1.19-1.16 (m, 2H), 1.08-1.06 (m, | |
| | | 2H) | |
| 182 | 2-(6-bromo-1-oxospiro[3H- | ¹H NMR (400 MHZ, DMSO-d₆): δ 10.60 (s, | m/z = |
| | isoquinoline-4,1′- | 1H), 8.18 (dd, J = 5.1, 0.5 Hz, 1H), 7.92-7.88 | 400.2, |
| | cyclopropane]-2-yl)-N-(4- | (m, 1H), 7.81 (d, J = 8.3 Hz, 1H), 7.53 (dd, J = | 402.2 |
| | methylpyridin-2- | 8.3, 1.9 Hz, 1H), 7.27 (d, J = 1.9 Hz, 1H), | [M + H]⁺ |
| | yl)acetamide | 6.95 (ddd, J = 5.1, 1.5, 0.7 Hz, 1H), 4.37 (s, | |
| | | 2H), 3.51 (s, 2H), 2.30 (s, 3H), 1.18-1.16 (m, | |
| | | 2H), 1.07-1.05 (m, 2H) | |
| 183 | 2-(6-bromo-1-oxospiro[3H- | | m/z = |
| | isoquinoline-4,1′- | | 497.0, |
| | cyclopropane]-2-yl)-N-(2- | | 499.0 |
| | chloro-4- | | [M + H]⁺ |
| | methylsulfonylphenyl) | | |
| | acetamide | | |
| 184 | 2-(6-bromo-1-oxospiro[3H- | ¹H NMR (400 MHZ, CDCl₃) δ 8.67 (br s, 1H), | m/z = |
| | isoquinoline-4,1′- | 8.32 (s, 1H), 8.02 (d, J = 8.4 Hz, 1H), 7.46 | 419.0, |
| | cyclopropane]-2-yl)-N-(5- | (dd, J = 8.4, 2.0 Hz, 1H), 7.00 (d, J = 1.6 Hz, | 421.0 |
| | fluoro-4-methylpyrimidin- | 1H), 4.62 (br s, 2H), 3.53 (s, 2H), 2.49 (d, J = | [M + H]⁺ |
| | 2-yl)acetamide | 2.4 Hz, 3H), 1.16-1.11 (m, 2H), 1.10-1.06 (m, | |
| | | 2H) | |
| 185 | 2-(6-bromo-1-oxospiro[3H- | ¹H NMR (400 MHZ, CDCl₃) δ 8.80 (br s, 1H), | m/z = |
| | isoquinoline-4,1′- | 8.30 (s, 2H), 8.02 (d, J = 8.4 Hz, 1H), 7.46 | 417.0, |
| | cyclopropane]-2-yl)-N-(5- | (dd, J = 8.4, 1.6 Hz, 1H), 7.00 (d, J = 1.6 Hz, | 419.0 |
| | methoxypyrimidin-2- | 1H), 4.59 (s, 2H), 3.90 (s, 3H), 3.54 (s, 2H), | [M + H]⁺ |
| | yl)acetamide | 1.15-1.10 (m, 2H), 1.09-1.04 (m, 2H) | |
| 186 | 2-(6-bromo-1-oxospiro[3H- | ¹H NMR (400 MHZ, CDCl₃): δ 8.68 (br s, | m/z = |
| | isoquinoline-4,1′- | 1H), 8.57 (s, 1H), 8.02 (d, J = 8.0 Hz, 1H), | 470.1, |
| | cyclopropane]-2-yl)-N-(3- | 7.95-7.92 (m, 2H), 7.77 (d, J = 1.2 Hz, 1H), | 472.1 |
| | fluoro-5-pyrazol-1- | 7.49-7.46 (m, 1H), 7.02 (d, J = 1.6 Hz, 1H), | [M + H]⁺ |
| | ylpyridin-2-yl)acetamide | 6.54-6.53 (m, 1H), 4.52 (s, 2H), 3.57 (s, 2H), | |
| | | 1.17-1.13 (m, 2H), 1.11-1.08 (m, 2H) | |
| 187 | 2-(6-bromo-1-oxospiro[3H- | ¹H NMR (400 MHZ, DMSO-d₆): δ 10.84 (br s, | m/z = |
| | isoquinoline-4,1′- | 1H), 8.74 (d, J = 5.2 Hz, 1H), 8.31 (s, 1H), | 484.0, |
| | cyclopropane]-2-yl)-N-[4- | 7.82 (d, J = 8.4 Hz, 1H), 7.68 (d, J = 5.2 Hz, | 486.0 |
| | (2-methyl-1,3-thiazol-4- | 1H), 7.53 (dd, J = 1.6, 8.4 Hz, 1H), 7.26 (d, J = | [M + H]⁺ |
| | yl)pyrimidin-2- | 1.6 Hz, 1H), 4.64 (s, 2H), 3.52 (s, 2H), 2.75 | |
| | yl]acetamide | (s, 3H), 1.21-1.13 (m, 2H), 1.11-1.03 (m, 2H) | |
| 188 | 2-(6-bromo-1-oxospiro[3H- | ¹H NMR (400 MHZ, DMSO-d₆): δ 10.46 (br s, | m/z = |
| | isoquinoline-4,1′- | 1H), 7.80 (d, J = 8.0 Hz, 1H), 7.52 (dd, J = | 416.0, |
| | cyclopropane]-2-yl)-N- | 1.6, 8.0 Hz, 1H), 7.25 (d, J = 2.0 Hz, 1H), | 418.0 |
| | (6,7-dihydro-5H- | 4.32 (br s, 2H), 4.03 (t, J = 7.2 Hz, 2H), 3.47 | [M + H]⁺ |
| | pyrrolo[1,2-b][1,2,4]triazol- | (s, 2H), 2.87-2.76 (m, 2H), 2.61-2.54 (m, 2H), | |
| | 2-yl)acetamide | 1.17-1.02 (m, 4H) | |
| 189 | 2-(6-bromo-1-oxospiro[3H- | ¹H NMR (400 MHZ, CDCl₃) δ 8.85 (br s, 1H), | m/z = |
| | isoquinoline-4,1′- | 8.33 (d, J = 9.2 Hz, 1H), 8.06 (s, 1H), 8.04- | 440.0, |
| | cyclopropane]-2-yl)-N-(1- | 8.03 (m, 1H), 7.79 (d, J = 9.2 Hz, 1H), 7.49 | 442.0 |
| | methylpyrazolo[4,3- | (d, J = 8.0 Hz, 1H), 7.02 (d, J = 1.6 Hz, 1H), | [M + H]⁺ |
| | b]pyridin-5-yl)acetamide | 4.42 (s, 2H), 4.10 (s, 3H), 3.54 (s, 2H), 1.20- | |
| | | 1.16 (m, 2H), 1.11-1.08 (m, 2H) | |
| 190 | 2-(6-bromo-1-oxospiro[3H- | ¹H NMR (400 MHZ, CDCl₃) δ 8.89 (br s, 1H), | m/z = |
| | isoquinoline-4,1′- | 8.22 (d, J = 9.2 Hz, 1H), 8.07-8.03 (m, 2H), | 440.0, |
| | cyclopropane]-2-yl)-N-(2- | 7.95 (s, 1H), 7.48 (dd, J = 8.4, 1.6 Hz, 1H), | 442.0 |
| | methylpyrazolo[4,3- | 7.01 (d, J = 1.6 Hz, 1H), 4.41 (s, 2H), 4.23 (s, | [M + H]⁺ |
| | b]pyridin-5-yl)acetamide | 3H), 3.54 (s, 2H), 1.18-1.14 (m, 2H), 1.12- | |
| | | 1.07 (m, 2H) | |
| 191 | 2-(6-bromo-1-oxospiro[3H- | ¹H NMR (400 MHZ, CDCl₃) δ 9.20-9.14 (m, | m/z = |
| | isoquinoline-4,1′- | 2H), 8.43 (d, J = 5.6 Hz, 1H), 8.37 (s, 1H), | 436.0, |
| | cyclopropane]-2-yl)-N- | 8.02 (d, J = 8.4 Hz, 1H), 7.74 (d, J = 8.8 Hz, | 438.0 |
| | isoquinolin-7-ylacetamide | 1H), 7.63 (dd, J = 8.8, 2.0 Hz, 1H), 7.56 (d, J = | [M + H]⁺ |
| | | 5.6 Hz, 1H), 7.49 (dd, J = 8.4, 2.0 Hz, 1H), | |
| | | 7.03 (d, J = 2.0 Hz, 1H), 4.38 (s, 2H), 3.62 (s, | |
| | | 2H), 1.17-1.12 (m, 2H), 1.11-1.06 (m, 2H) | |
| 192 | 2-(6-bromo-1-oxospiro[3H- | ¹H NMR (400 MHZ, CDCl₃): δ 8.87 (s, 2H), | m/z = |
| | isoquinoline-4,1′- | 8.80 (br s, 1H), 7.93 (d, J = 8.4 Hz, 1H), 7.40- | 388.0, |
| | cyclopropane]-2-yl)-N- | 7.37 (m, 1H), 6.93 (s, 1H), 4.61 (s, 2H), 3.45 | 390.0 |
| | (1,3,5-triazin-2- | (s, 2H), 1.06-1.04 (m, 2H), 1.02-0.98 (m, 2H) | [M + H]⁺ |
| | yl)acetamide | | |

### Example 193

### 2-(6-bromo-1-oxospiro[3H-isoquinoline-4,1′-cyclopropane]-2-yl)-N-[5-(trifluoromethyl)pyrimidin-2-yl]acetamide (193)

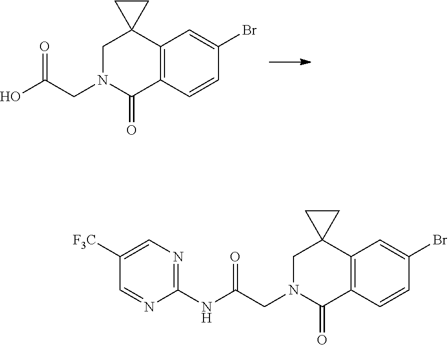 [SMILES: O=C(O)CN1CC2(CC2)c2cc(Br)ccc2C1=O ; O=C(CN1CC2(CC2)c2cc(Br)ccc2C1=O)Nc1ncc(C(F)(F)F)cn1]

To a solution of 2-(6-bromo-1-oxo-spiro[3H-isoquinoline-4,1′-cyclopropane]-2-yl)acetic acid (38 mg, 0.12 mmol) in MeCN (1.0 mL) were added 5-(trifluoromethyl)pyrimidin-2-amine (40 mg, 0.25 mmol), 1-methylimidazole (70 mg, 0.86 mmol), and chloro-N,N,N′,N′-tetramethylformamidinium hexafluorophosphate (69 mg, 0.25 mmol). The reaction mixture was stirred at 23° C. for 24 h. The reaction mixture was purified directly by reverse-phase preparative HPLC. LCMS: m/z=455.2, 457.2 [M+H]⁺. ¹H NMR (400 MHz, CDCl₃): δ 9.07 (s, 1H), 8.83 (d, J=0.8 Hz, 2H), 8.01 (d, 1=8.3 Hz, 1H), 7.46 (dd, J=8.3, 1.9 Hz, 1H), 7.00 (d, J=1.8 Hz, 1H), 4.65 (s, 2H), 3.53 (s, 2H), 1.15-1.11 (m, 2H), 1.08-1.05 (m, 2H).

The following compound was, or can be, made via similar procedures as those described above:

| Ex. | Name | NMR | LCMS |
|---|---|---|---|
| 194 | 2-(6-bromo-1-oxospiro[3H- | ¹H NMR (400 MHZ, CDCl₃): δ 9.59 (s, 1H), | m/z = |
| | isoquinoline-4,1′- | 8.38 (s, 2H), 7.98 (d, J = 8.3 Hz, 1H), 7.42 | 427.1, |
| | cyclopropane]-2-yl)-N-(5- | (dd, J = 8.3, 1.8 Hz, 1H), 6.98 (d, J = 1.8 Hz, | 429.1 |
| | cyclopropylpyrimidin-2- | 1H), 4.68 (s, 2H), 3.51 (s, 2H), 1.86-1.79 (m, | [M + H]⁺ |
| | yl)acetamide | 1H), 1.12-1.02 (m, 6H), 0.74-0.70 (m, 2H) | |
| 195 | 2-(6-bromo-1-oxospiro[3H- | ¹H NMR (400 MHZ, CDCl₃): δ 9.15 (s, 1H), | m/z = |
| | isoquinoline-4,1′- | 8.40 (s, 1H), 8.30-8.28 (m, 1H), 8.02 (d, J = | 436.0, |
| | cyclopropane]-2-yl)-N-[5- | 8.4 Hz, 1H), 7.89-7.83 (m, 1H), 7.47 (dd, J = | 438.1 |
| | (difluoromethyl)pyridin-2- | 8.4, 1.9 Hz, 1H), 7.01 (d, J = 1.9 Hz, 1H), | [M + H]⁺ |
| | yl]acetamide | 6.66 (t, J = 55.9 Hz, 1H), 4.39 (s, 2H), 3.52 (s, | |
| | | 2H), 1.17-1.11 (m, 2H), 1.08-1.04 (m, 2H) | |
| 196 | 2-(6-bromo-1-oxospiro[3H- | ¹H NMR (400 MHZ, CDCl₃): δ 8.78 (d, J = | m/z = |
| | isoquinoline-4,1′- | 0.3 Hz, 1H), 8.22 (dd, J = 2.6, 0.7 Hz, 1H), | 420.2, |
| | cyclopropane]-2-yl)-N-(5- | 8.17-8.14 (m, 1H), 8.02 (d, J = 8.4 Hz, 1H), | 422.2, |
| | chloropyridin-2- | 7.66 (dd, J = 8.9, 2.6 Hz, 1H), 7.47 (dd, J = | [M + H]⁺ |
| | yl)acetamide | 8.3, 1.9 Hz, 1H), 7.01 (d, J = 1.8 Hz, 1H), | |
| | | 4.35 (s, 2H), 3.51 (s, 2H), 1.18-1.14 (m, 2H), | |
| | | 1.08-1.04 (m, 2H) | |
| 197 | 2-(6-bromo-1-oxospiro[3H- | ¹H NMR (400 MHZ, CDCl₃): δ 9.52 (s, 1H), | m/z = |
| | isoquinoline-4,1′- | 8.04-7.90 (m, 5H), 7.65 (t, J = 7.2 Hz, 1H), | 437.3, |
| | cyclopropane]-2-yl)-N- | 7.44-7.42 (m, 1H), 6.99 (d, J = 1.8 Hz, 1H), | 439.2 |
| | quinazolin-2-ylacetamide | 4.90 (s, 2H), 3.56 (s, 2H), 1.11-1.08 (m, 4H) | [M + H]⁺ |
| 198 | 2-(6-bromo-1-oxospiro[3H- | ¹H NMR (400 MHZ, CDCl₃): δ 8.47-8.36 (m, | m/z = |
| | isoquinoline-4,1′- | 2H), 8.02 (d, J = 8.3 Hz, 1H), 7.91-7.86 (m, | 436.2, |
| | cyclopropane]-2-yl)-N- | 2H), 7.82-7.77 (m, 1H), 7.61-7.57 (m, 1H), | 438.2 |
| | quinolin-2-ylacetamide | 7.46 (dd, J = 8.3, 1.9 Hz, 1H), 7.01 (d, J = 1.9 | [M + H]⁺ |
| | | Hz, 1H), 4.57 (s, 2H), 3.54 (s, 2H), 1.18-1.14 | |
| | | (m, 2H), 1.11-1.08 (m, 2H) | |
| 199 | 2-(6-bromo-1-oxospiro[3H- | ¹H NMR (400 MHZ, CDCl₃): δ 10.65 (s, 1H), | m/z = |
| | isoquinoline-4,1′- | 9.66 (s, 1H), 8.48 (d, J = 9.0 Hz, 1H), 8.20 | 437.3, |
| | cyclopropane]-2-yl)-N- | (dd, J = 8.1, 1.8 Hz, 1H), 8.04-7.98 (m, 2H), | 439.2 |
| | (1,8-naphthyridin-2- | 7.86 (s, 1H), 7.45 (dd, J = 8.3, 1.9 Hz, 1H), | [M + H]⁺ |
| | yl)acetamide | 7.01 (d, J = 1.8 Hz, 1H), 4.82-4.81 (m, 2H), | |
| | | 3.58 (s, 2H), 1.17-1.13 (m, 2H), 1.11-1.07 (m, | |
| | | 2H) | |
| 200 | N-(1,2-benzoxazol-5-yl)-2- | ¹H NMR (400 MHZ, CD₃CN): δ 8.82 (d, J = | m/z = |
| | (6-bromo-1-oxospiro[3H- | 0.9 Hz, 1H), 8.68 (s, 1H), 8.20 (d, J = 1.3 Hz, | 426.2, |
| | isoquinoline-4,1′- | 1H), 7.91 (d, J = 8.3 Hz, 1H), 7.62 (d, J = 2.0 | 428.2 |
| | cyclopropane]-2- | Hz, 1H), 7.61 (t, J = 0.9 Hz, 1H), 7.49 (dd, J = | [M + H]⁺ |
| | yl)acetamide | 8.3, 1.9 Hz, 1H), 7.17 (d, J = 1.9 Hz, 1H), | |
| | | 4.31 (s, 2H), 3.52 (s, 2H), 1.16-1.12 (m, 2H), | |
| | | 1.10-1.06 (m, 2H) | |
| 201 | 2-(6-bromo-1-oxospiro[3H- | ¹H NMR (400 MHZ, CDCl₃): δ 8.94 (s, 1H), | m/z = |
| | isoquinoline-4,1′- | 8.91 (d, J = 1.2 Hz, 1H), 8.07 (s, 1H), 7.97 (d, | 425.3, |
| | cyclopropane]-2-yl)-N- | J = 8.3 Hz, 1H), 7.47 (dd, J = 8.3, 1.9 Hz, | 427.3 |
| | imidazo[1,5-a]pyridin-6- | 1H), 7.37 (s, 1H), 7.33 (d, J = 9.6 Hz, 1H), | [M + H]⁺ |
| | ylacetamide | 7.01 (d, J = 1.8 Hz, 1H), 6.45 (dd, J = 9.6, 1.6 | |
| | | Hz, 1H), 4.31 (s, 2H), 3.58 (s, 2H), 1.16-1.11 | |
| | | (m, 2H), 1.08-1.04 (m, 2H) | |
| 202 | 2-(6-bromo-1-oxospiro[3H- | ¹H NMR (400 MHZ, CDCl₃): δ 9.63 (s, 1H), | m/z = |
| | isoquinoline-4,1′- | 8.51 (s, 2H), 7.97 (d, J = 8.3 Hz, 1H), 7.44 | 401.3, |
| | cyclopropane]-2-yl)-N-(5- | (dd, J = 8.3, 1.7 Hz, 1H), 6.98 (d, J = 1.7 Hz, | 403.2 |
| | methylpyrimidin-2- | 1H), 4.66 (s, 2H), 3.51 (s, 2H), 2.30 (s, 3H), | [M + H]⁺ |
| | yl)acetamide | 1.13-1.04 (m, 4H) | |
| 203 | N-(1,2-benzoxazol-6-yl)-2- | ¹H NMR (400 MHZ, CD₃CN): δ 8.85 (s, 1H), | m/z = |
| | (6-bromo-1-oxospiro[3H- | 8.77 (d, J = 1.1 Hz, 1H), 8.20-8.18 (m, 1H), | 426.2, |
| | isoquinoline-4,1′- | 7.91 (dd, J = 8.3, 0.3 Hz, 1H), 7.73 (dd, J = | 428.2 |
| | cyclopropane]-2- | 8.6, 0.6 Hz, 1H), 7.49 (dd, J = 8.3, 1.9 Hz, | [M + H]⁺ |
| | yl)acetamide | 1H), 7.35 (dd, J = 8.6, 1.7 Hz, 1H), 7.17 (d, J = | |
| | | 1.7 Hz, 1H), 4.33 (s, 2H), 3.52 (s, 2H), 1.16- | |
| | | 1.12 (m, 2H), 1.10-1.06 (m, 2H) | |
| 204 | 2-(6-bromo-1-oxospiro[3H- | ¹H NMR (400 MHZ, CDCl₃): δ 8.82 (s, 1H), | m/z = |
| | isoquinoline-4,1′- | 8.44 (s, 2H), 8.01 (d, J = 8.3 Hz, 1H), 7.45 | 415.4, |
| | cyclopropane]-2-yl)-N-(5- | (dd, J = 8.3, 1.9 Hz, 1H), 6.99 (d, J = 1.8 Hz, | 417.3 |
| | ethylpyrimidin-2- | 1H), 4.65-4.64 (m, 2H), 3.52 (s, 2H), 2.64- | [M + H]⁺ |
| | yl)acetamide | 2.58 (m, 2H), 1.25 (t, J = 7.6 Hz, 3H), 1.14- | |
| | | 1.10 (m, 2H), 1.10-1.05 (m, 2H) | |
| 205 | N-(1,3-benzoxazol-2-yl)-2- | ¹H NMR (400 MHZ, acetone-d₆): δ 7.94 (d, J = | m/z = |
| | (6-bromo-1-oxospiro[3H- | 8.3 Hz, 1H), 7.57-7.50 (m, 3H), 7.35-7.26 | 426.2, |
| | isoquinoline-4,1′- | (m, 2H), 7.21 (d, J = 1.9 Hz, 1H), 4.76 (s, | 428.2 |
| | cyclopropane]-2- | 2H), 3.65 (s, 2H), 1.21-1.20 (m, 2H), 1.18- | [M + H]⁺ |
| | yl)acetamide | 1.16 (m, 2H) | |
| 206 | 2-(6-bromo-1-oxospiro[3H- | ¹H NMR (400 MHZ, CDCl₃): δ 9.97 (s, 1H), | m/z = |
| | isoquinoline-4,1′- | 9.40 (s, 1H), 8.81 (d, J = 9.3 Hz, 1H), 8.61 (d, | 437.3, |
| | cyclopropane]-2-yl)-N- | J = 6.7 Hz, 1H), 8.56 (d, J = 8.6 Hz, 1H), | 439.2 |
| | (1,6-naphthyridin-2- | 8.12-8.10 (m, 1H), 8.05 (d, J = 8.3 Hz, 1H), | [M + H]⁺ |
| | yl)acetamide | 7.52-7.49 (m, 1H), 7.03 (d, J = 1.8 Hz, 1H), | |
| | | 4.44 (s, 2H), 3.58 (s, 2H), 1.19-1.16 (m, 2H), | |
| | | 1.10-1.06 (m, 2H) | |
| 207 | 2-(6-bromo-1-oxospiro[3H- | ¹H NMR (400 MHZ, DMSO-d₆): δ 12.35- | m/z = |
| | isoquinoline-4,1′- | 12.33 (m, 1H), 8.28 (t, J = 0.3 Hz, 1H), 7.81 | 451.2, |
| | cyclopropane]-2-yl)-N-(6- | (d, J = 8.3 Hz, 1H), 7.79-7.73 (m, 2H), 7.53 | 453.1 |
| | cyano-1,3-benzoxazol-2- | (dd, J = 8.4, 1.9 Hz, 1H), 7.28 (d, J = 1.9 Hz, | [M + H]⁺ |
| | yl)acetamide | 1H), 4.57 (d, J = 0.2 Hz, 2H), 3.52 (s, 2H), | |
| | | 1.20-1.17 (m, 2H), 1.08-1.05 (m, 2H) | |
| 208 | 2-(6-bromo-1-oxospiro[3H- | ¹H NMR (400 MHZ, CDCl₃): δ 8.92 (s, 1H), | m/z = |
| | isoquinoline-4,1′- | 8.22 (dd, J = 9.1, 4.1 Hz, 1H), 8.15 (d, J = 3.0 | 404.2, |
| | cyclopropane]-2-yl)-N-(5- | Hz, 1H), 8.04 (d, J = 8.3 Hz, 1H), 7.50-7.44 | 406.1 |
| | fluoropyridin-2- | (m, 2H), 7.03 (d, J = 1.9 Hz, 1H), 4.39 (s, | [M + H]⁺ |
| | yl)acetamide | 2H), 3.54 (s, 2H), 1.20-1.16 (m, 2H), 1.11- | |
| | | 1.07 (m, 2H) | |
| 209 | 2-(6-bromo-1-oxo- | ¹H NMR (400 MHZ, DMSO-d₆): δ 12.30 (s, | m/z = |
| | spiro[3H-isoquinoline-4,1′- | 1H), 8.39 (dd, J = 5.0, 1.4 Hz, 1H), 8.07 (dd, J = | 427.2, |
| | cyclopropane]-2-yl)-N- | 8.1, 1.4 Hz, 1H), 7.82 (d, J = 8.3 Hz, 1H), | 429.1 |
| | oxazolo[4,5-b]pyridin-2-yl- | 7.54 (dd, J = 8.3, 1.9 Hz, 1H), 7.32-7.28 (m, | [M + H]⁺ |
| | acetamide | 2H), 4.58 (dd, J = 3.6, 0.5 Hz, 2H), 3.53 (s, | |
| | | 2H), 1.20-1.17 (m, 2H), 1.08-1.05 (m, 2H) | |
| 210 | 2-(6-bromo-1-oxo- | ¹H NMR (400 MHZ, DMSO-d₆): δ 12.71 (s, | m/z = |
| | spiro[3H-isoquinoline-4,1′- | 1H), 9.26 (s, 1H), 8.76 (d, J = 6.2 Hz, 1H), | 427.2, |
| | cyclopropane]-2-yl)-N- | 8.25-8.24 (m, 1H), 7.82 (d, J = 8.3 Hz, 1H), | 429.1 |
| | oxazolo[4,5-c]pyridin-2-yl- | 7.54 (dd, J = 8.3, 1.9 Hz, 1H), 7.29 (d, J = 1.9 | [M + H]⁺ |
| | acetamide | Hz, 1H), 4.58 (s, 2H), 3.54 (s, 2H), 1.21-1.18 | |
| | | (m, 2H), 1.09-1.06 (m, 2H) | |
| 211 | 2-(6-bromo-1-oxo- | ¹H NMR (400 MHZ, DMSO-d₆): δ 12.16 (s, | m/z = |
| | spiro[3H-isoquinoline-4,1′- | 1H), 8.21-8.19 (m, 1H), 8.03-8.01 (m, 1H), | 427.2, |
| | cyclopropane]-2-yl)-N- | 7.82 (d, J = 8.3 Hz, 1H), 7.54 (dd, J = 8.3, 1.9 | 429.1 |
| | oxazolo[5,4-b]pyridin-2-yl- | Hz, 1H), 7.41 (dd, J = 7.8, 5.0 Hz, 1H), 7.28 | [M + H]⁺ |
| | acetamide | (d, J = 2.0 Hz, 1H), 4.56 (dt, J = 2.1, 0.9 Hz, | |
| | | 2H), 3.52 (s, 2H), 1.19-1.16 (m, 2H), 1.08- | |
| | | 1.05 (m, 2H) | |
| 212 | 2-(6-bromo-1-oxo- | ¹H NMR (400 MHZ, DMSO-d₆): δ 9.24-9.23 | m/z = |
| | spiro[3H-isoquinoline-4,1′- | (m, 1H), 8.65-8.63 (m, 1H), 7.95-7.93 (m, | 427.2, |
| | cyclopropane]-2-yl)-N- | 1H), 7.82 (d, J = 8.3 Hz, 1H), 7.55-7.53 (m, | 429.1 |
| | oxazolo[5,4-c]pyridin-2-yl- | 1H), 7.29 (t, J = 1.8 Hz, 1H), 4.59 (s, 2H), | [M + H]⁺ |
| | acetamide | 3.53 (s, 2H), 1.21-1.17 (m, 2H), 1.08-1.05 (m, | |
| | | 2H) | |
| 213 | 2-(6-bromo-1-oxospiro[3H- | ¹H NMR (400 MHZ, DMSO-d₆): δ 10.89 (s, | m/z = |
| | isoquinoline-4,1′- | 1H), 8.61 (d, J = 0.9 Hz, 1H), 7.81 (dd, J = | 429.3, |
| | cyclopropane]-2-yl)-N- | 8.3, 2.4 Hz, 1H), 7.52 (ddd, J = 8.3, 6.7, 1.9 | 431.2 |
| | (5,7-dihydrofuro[3,4- | Hz, 1H), 7.27-7.24 (m, 1H), 5.07-5.06 (m, | [M + H]⁺ |
| | d]pyrimidin-2-yl)acetamide | 2H), 4.91-4.90 (m, 2H), 4.55-4.54 (m, 2H), | |
| | | 3.50-3.44 (m, 2H), 1.18-1.12 (m, 2H), 1.07- | |
| | | 1.01 (m, 2H) | |
| 214 | 2-(6-bromo-1-oxospiro[3H- | ¹H NMR (400 MHZ, DMSO-d₆): δ 8.29 (d, J = | m/z = |
| | isoquinoline-4,1′- | 0.3 Hz, 1H), 7.82-7.80 (m, 1H), 7.77-7.74 (m, | 403.2, |
| | cyclopropane]-2-yl)-N-(6- | 1H), 7.54-7.50 (m, 1H), 7.27 (d, J = 1.9 Hz, | 405.2 |
| | oxo-1H-pyrimidin-2- | 1H), 6.60-6.57 (m, 1H), 5.98-5.95 (m, 1H), | [M + H]⁺ |
| | yl)acetamide | 4.38 (s, 2H), 3.50 (s, 2H), 1.19-1.12 (m, 2H), | |
| | | 1.08-1.03 (m, 2H) | |
| 215 | 2-(6-fluoro-1-oxospiro[3H- | ¹H NMR (400 MHZ, CDCl₃): δ 9.01 (s, 1H), | m/z = |
| | isoquinoline-4,1′- | 8.49 (s, 2H), 8.17 (dd, J = 8.7, 5.9 Hz, 1H), | 345.1 |
| | cyclopropane]-2-yl)-N-(5- | 6.99 (td, J = 8.5, 2.5 Hz, 1H), 6.54 (dd, J = | [M + H]⁺ |
| | fluoropyrimidin-2- | 9.6, 2.5 Hz, 1H), 4.56 (s, 2H), 3.55 (s, 2H), | |
| | yl)acetamide | 1.10 (dt, J = 2.9, 1.5 Hz, 2H), 1.09-1.06 (m, | |
| | | 2H) | |
| 216 | 2-(6-bromo-1-oxospiro[3H- | | m/z = |
| | isoquinoline-4,1′- | | 415.0, |
| | cyclopropane]-2-yl)-N- | | 417.0 |
| | (2,6-dimethylpyrimidin-4- | | [M + H]⁺ |
| | yl)acetamide | | |
| 217 | 2-(6-bromo-1-oxospiro[3H- | | m/z = |
| | isoquinoline-4,1′- | | 415.0, |
| | cyclopropane]-2-yl)-N- | | 417.0 |
| | (4,5-dimethylpyrimidin-2- | | [M + H]⁺ |
| | yl)acetamide | | |
| 218 | 2-(6-bromo-1-oxospiro[3H- | | m/z = |
| | isoquinoline-4,1′- | | 390.1, |
| | cyclopropane]-2-yl)-N-(2- | | 392.1 |
| | methyl-1,2,4-triazol-3- | | [M + H]⁺ |
| | yl)acetamide | | |
| 219 | 2-(6-bromo-1-oxospiro[3H- | ¹H NMR (400 MHZ, CDCl₃): δ 10.31-10.19 | m/z = |
| | isoquinoline-4,1′- | (m, 1H), 8.01 (d, J = 8.4 Hz, 1H), 7.90 (t, J = | 451.2, |
| | cyclopropane]-2-yl)-N-(5- | 0.7 Hz, 1H), 7.56 (dd, J = 8.4, 1.6 Hz, 1H), | 453.2 |
| | cyano-1,3-benzoxazol-2- | 7.51 (dd, J = 8.4, 0.6 Hz, 1H), 7.49 (dd, J = | [M + H]⁺ |
| | yl)acetamide | 8.3, 1.9 Hz, 1H), 7.02 (d, J = 1.8 Hz, 1H), | |
| | | 4.47 (t, J = 0.7 Hz, 2H), 3.58 (s, 2H), 1.17- | |
| | | 1.12 (m, 2H), 1.09-1.05 (m, 2H) | |

### Example 220

### 2-[(2′s,4r)-6-bromo-2′,5-difluoro-1-oxospiro[3H-isoquinoline-4,1′-cyclopropane]-2-yl]-N-(5-fluoropyrimidin-2-yl)acetamide (220)

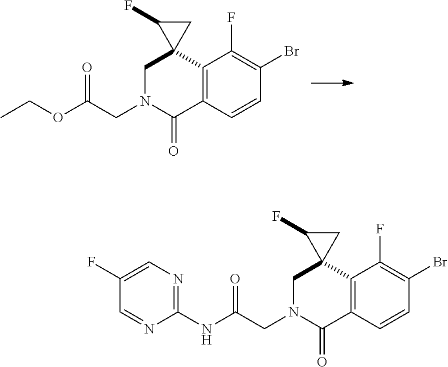

To a solution of ethyl 2-[(2′s,4r)-6-bromo-2′,5-difluoro-1-oxo-spiro[3H-isoquinoline-4,1′-cyclopropane]-2-yl]acetate (20 mg, 0.05 mmol, Int. 40) and 5-fluoropyrimidin-2-amine (12 mg, 0.11 mmol) in DCE (1 mL) was added AlMe₃ (1 M in heptane, 0.12 mL). The mixture was heated to 90° C. and stirred for 2 h. The mixture was quenched with H₂O (10 mL) and extracted with EtOAc (3×3 mL). The combined organic layers were washed with brine (3 mL) and concentrated under reduced pressure. The residue was purified by reverse-phase preparative HPLC. LCMS: m/z=440.9, 442.9 [M+H]⁺. ¹H NMR (400 MHz, CDCl₃): δ 8.72 (br d, J=2.8 Hz, 1H), 8.48 (s, 2H), 7.90 (dd, J=0.9, 8.4 Hz, 1H), 7.56 (dd, J=6.4, 8.4 Hz, 1H), 5.44-5.18 (m, 1H), 5.01-4.85 (m, 1H), 4.45 (br d, J=16.9 Hz, 1H), 3.92 (dd, J=2.6, 13.1 Hz, 1H), 3.68 (d, J=13.0 Hz, 1H), 1.96 (td, J=6.9, 11.9 Hz, 1H), 1.54-1.44 (m, 1H).

### Example 221

### 2-[(2′s,4r)-6-bromo-2′,5-difluoro-1-oxospiro[3H-isoquinoline-4,1′-cyclopropane]-2-yl]-N-(5-cyanopyrimidin-2-yl)acetamide (221)

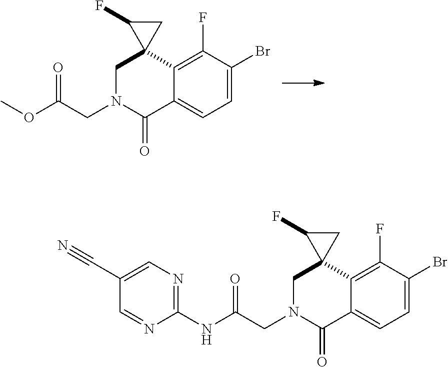

To a solution of methyl 2-[(2′s,4r)-6-bromo-2′,5-difluoro-1-oxo-spiro[3H-isoquinoline-4,1′-cyclopropane]-2-yl]acetate (50 mg, 0.14 mmol, Int. 43) and 2-aminopyrimidine-5-carbonitrile (33 mg, 0.28 mmol) in DCE (1 mL) 0° C. was added AlMe₃ (1 M in heptane, 0.3 mL). The mixture was stirred at 60° C. for 2 h. The reaction mixture was quenched with H₂O (3 mL) and extracted with EtOAc (4×3 mL). The combined organic layers were washed with brine (5 mL), dried over anhydrous Na₂SO₄, filtered, concentrated under reduced pressure, and purified by reverse-phase preparative HPLC. LCMS: m/z=447.9, 449.9 [M+H]⁺. ¹H NMR (400 MHz, CDCl₃): δ 9.06 (br s, 1H), 8.86 (s, 2H), 7.89 (d, J=8.4 Hz, 1H), 7.57 (dd, J=6.4, 8.4 Hz, 1H), 5.42-5.19 (m, 1H), 4.98 (d, J=17.2 Hz, 1H), 4.52 (d, J=17.2 Hz, 1H), 3.92 (dd, J=2.4, 13.0 Hz, 1H), 3.67 (d, J=13.2 Hz, 1H), 1.98 (td, J=7.2, 11.6 Hz, 1H), 1.54-1.44 (m, 1H).

### Example 222

### 2-[(2′s,4r)-6-bromo-2′,5-difluoro-1-oxospiro[3H-isoquinoline-4,1′-cyclopropane]-2-yl]-N-(5-chloropyrimidin-2-yl)acetamide (222)

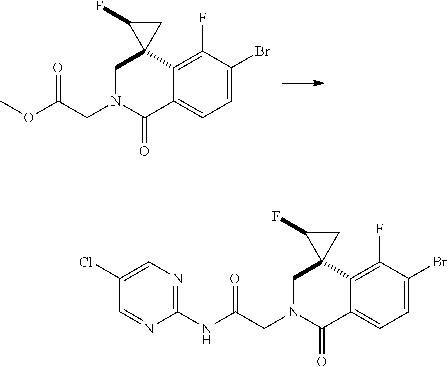

To a solution of methyl 2-[(2′s,4r)-6-bromo-2′,5-difluoro-1-oxo-spiro[3H-isoquinoline-4,1′-cyclopropane]-2-yl]acetate (50 mg, 0.14 mmol, Int. 43) and 5-chloropyrimidin-2-amine (36 mg, 0.28 mmol) in DCE (1 mL) was added AlMe₃ (1 M in n-heptane, 0.31 mL). The mixture was heated to 85° C., and stirred for 2 h. The reaction mixture was quenched with H₂O (8 mL) at 0° C., and extracted with EtOAc (3×5 mL). The combined organic layers were washed with brine (15 mL), dried over anhydrous Na₂SO₄, filtered, concentrated under reduced pressure, and purified by reverse-phase preparative HPLC. LCMS: m/z=456.9, 458.9, 460.9 [M+H]⁺. ¹H NMR (400 MHz, CDCl₃): δ 8.90 (br s, 1H), 8.56 (s, 2H), 7.89 (d, J=8.4 Hz, 1H), 7.63-7.45 (m, 1H), 5.30-5.27 (m, 1H), 4.98 (br d, J=16.4 Hz, 1H), 4.48 (br d, J=16.8 Hz, 1H), 3.91 (dd, J=2.4, 13.2 Hz, 1H), 3.67 (d, J=12.8 Hz, 1H), 1.95 (td, J=7.4, 11.6 Hz, 1H), 1.56-1.45 (m. 1H).

### Example 223

### 2-[(2′s,4r)-6-chloro-2′,5-difluoro-1-oxospiro[3H-isoquinoline-4,1′-cyclopropane]-2-yl]-N-(5-fluoropyrimidin-2-yl)acetamide (223)

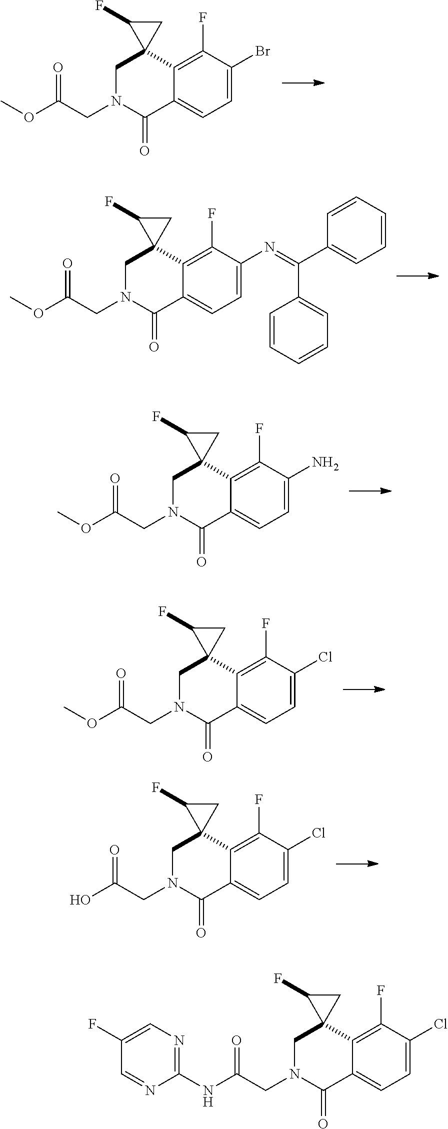

methyl 2-[(2′s,4r)-2′,5-difluoro-6-((diphenylmethylene)amino)-1-oxo-spiro[3H-isoquinoline-4,1′-cyclopropane]-2-yl]acetate: To a solution of methyl 2-[(2′s,4r)-6-bromo-2′,5-difluoro-1-oxo-spiro[3H-isoquinoline-4,1′-cyclopropane]-2-yl]acetate (300 mg, 0.83 mmol, Int. 43) and diphenylmethanimine (302 mg, 1.67 mmol) in 1,4-dioxane (10 mL) were added Pd₂(dba)₃ (76 mg, 0.08 mmol), XPhos (80 mg, 0.17 mmol) and Cs₂CO₃ (543 mg, 1.67 mmol). The mixture was stirred at 90° C. for 16 h. The mixture was cooled to 20° C., and added to sat. aq. NH₄Cl (10 mL). The mixture was extracted with EtOAc (5×10 mL). The combined organic layers were washed with brine (10 mL), dried over anhydrous Na₂SO₄, filtered and concentrated under reduced pressure to provide a residue that was used directly. LCMS: m/z=461.1 [M+H]⁺.

methyl 2-[(2′s,4r)-6-amino-2′,5-difluoro-1-oxo-spiro[3H-isoquinoline-4,1′-cyclopropane]-2-yl]acetate: A solution of methyl 2-[(2′s,4r)-2′,5-difluoro-6-((diphenylmethylene)amino)-1-oxo-spiro[3H-isoquinoline-4,1′-cyclopropane]-2-yl]acetate (650 mg, 1.41 mmol), NH₂OH·HCl (196 mg, 2.82 mmol), and NaOAc (347 mg, 4.23 mmol) in MeOH (10 mL) was stirred for 16 h. The reaction mixture was poured into sat. aq. NaHCO₃ (15 mL). The mixture was extracted with EtOAc (3×10 mL). The combined organic layers were washed with brine (10 mL), dried over anhydrous Na₂SO₄, filtered, concentrated under reduced pressure, and purified by silica gel column chromatography. LCMS: m/z=297.2 [M+H]⁺.

methyl 2-[(2′s,4r)-6-chloro-2′,5-difluoro-1-oxo-spiro[3H-isoquinoline-4,1′-cyclopropane]-2-yl]acetate: To a mixture of CuCl (107 mg, 1.08 mmol) in MeCN (10 mL) at 60° C. were added t-BuONO (100 mg, 0.97 mmol) and a solution of methyl 2-[(2′s,4r)-6-amino-2′,5-difluoro-1-oxo-spiro[3H-isoquinoline-4,1′-cyclopropane]-2-yl]acetate (160 mg, 0.54 mmol) in MeCN (3 mL). The mixture was stirred at 60° C. for 5 h. The reaction mixture was poured into sat. aq. NaHCO₃ (10 mL) and the mixture was extracted with EtOAc (3×10 mL). The combined organic layers were washed with brine (10 mL), dried over anhydrous Na₂SO₄, filtered, concentrated under reduced pressure, and purified by silica gel column chromatography. LCMS: m/z=316.1, 318.1 [M+H]⁺.

2-[(2′s,4r)-6-chloro-2′,5-difluoro-1-oxo-spiro[3H-isoquinoline-4,1′-cyclopropane]-2-yl]acetic acid: A mixture of methyl 2-[(2′s,4r)-6-chloro-2′,5-difluoro-1-oxo-spiro[3H-isoquinoline-4,1′-cyclopropane]-2-yl]acetate (43 mg, 0.13 mmol) and LiOH·H₂O (12 mg, 0.27 mmol) in THF (2 mL) and H₂O (2 mL) was stirred for 1 h. The reaction mixture was diluted with H₂O (5 mL). The mixture was washed with MTBE (3 mL). The aqueous layer was adjusted to pH=1 with HCl (2M) and extracted with DCM (3×5 mL). The combined organic layers were dried over anhydrous Na₂SO₄, filtered and concentrated to provide a residue that was used directly. ¹H NMR (400 MHz, CDCl₃): δ 7.94 (d, J=8.4 Hz, 1H), 7.39 (dd, J=8.8 Hz, 1H), 5.46-5.19 (m, 1H), 4.64 (d, J=17.6 Hz, 1H), 4.11 (d, J=17.6 Hz, 1H), 3.86-3.77 (m, 1H), 3.65-3.62 (m, 1H), 1.94-1.87 (m, 1H), 1.53-1.43 (m, 1H).

2-[(2′s,4r)-6-chloro-2′,5-difluoro-1-oxospiro[3H-isoquinoline-4,1′-cyclopropane]-2-yl]-N-(5-fluoropyrimidin-2-yl)acetamide: To a solution of 5-fluoropyrimidin-2-amine (25 mg, 0.22 mmol) and 2-[(2′s,4r)-6-chloro-2′,5-difluoro-1-oxo-spiro[3H-isoquinoline-4,1′-cyclopropane]-2-yl]acetic acid (33 mg, 0.11 mmol) in pyridine (1.0 mL) was added EDCI (31 mg, 0.16 mmol). The mixture was stirred at 15° C. for 16 h. The reaction mixture was poured into sat. aq. NH₄Cl (10 mL) and the mixture was extracted with EtOAc (3×5 mL). The combined organic layers were washed with brine (5 mL), dried over anhydrous Na₂SO₄, filtered, concentrated under reduced pressure, and purified by reverse-phase preparative HPLC. LCMS: m/z=397.1, 399.1 [M+H]⁺. ¹H NMR (400 MHz, CDCl₃): δ 8.69 (br s, 1H), 8.48 (s, 2H), 7.97 (d, J=8.4 Hz, 1H), 7.40 (m, 1H), 5.40-5.22 (m, 1H), 4.92 (d, J=16.8 Hz, 1H), 4.46-4.42 (m, 1H), 3.94-3.91 (m, 1H), 3.69-3.66 (m, 1H), 2.04-1.87 (m, 1H), 1.49 (m, 1H).

### Example 224

### 2-[(2′s,4r)-6-bromo-2′,5-difluoro-1-oxospiro[3H-isoquinoline-4,1′-cyclopropane]-2-yl]-N-pyrimidin-2-ylacetamide (224)

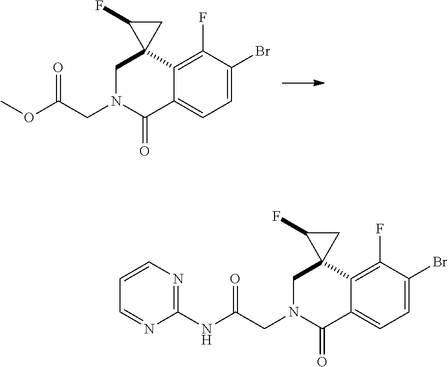

To a mixture of methyl 2-[(2′s,4r)-6-bromo-2′,5-difluoro-1-oxo-spiro[3H-isoquinoline-4,1′-cyclopropane]-2-yl]acetate (30 mg, 0.08 mmol, Int. 43) and pyrimidin-2-amine (16 mg, 0.17 mmol) in DCM (2 mL) was added AlMe₃ (1 M in heptane, 0.18 mL). The mixture was stirred at 90° C. for 2 h. The reaction mixture was diluted with sat. aq. NH₄Cl (10 mL) and extracted with EtOAc (3×5 mL). The combined organic layers were washed with brine (5 mL), dried over anhydrous Na₂SO₄, filtered, concentrated under reduced pressure and purified by reverse-phase preparative HPLC. LCMS: m/z=423.0, 424.9 [M+H]⁺. ¹H NMR (400 MHz, CDCl₃): δ=9.16 (br s, 1H), 8.63 (d, J=4.8 Hz, 2H), 7.89 (d, J=8.4 Hz, 1H), 7.54 (dd, J=6.4, 8.4 Hz, 1H), 7.04 (t, J=4.8 Hz, 1H), 5.46-5.23 (m, TH), 5.10 (br d, J=16.8 Hz, 1H), 4.57 (br d, J=17.2 Hz, 1H), 3.90 (d, J=13.2 Hz, 1H), 3.69 (d, J=13.2 Hz, 1H), 1.96-1.89 (m, 1H), 1.58-1.45 (m, 1H).

### Example 225

### 2-[(2′s,4r)-6-bromo-2′,5-difluoro-1-oxospiro[3H-isoquinoline-4,1′-cyclopropane]-2-yl]-N-(5-methylpyrimidin-2-yl)acetamide (225)

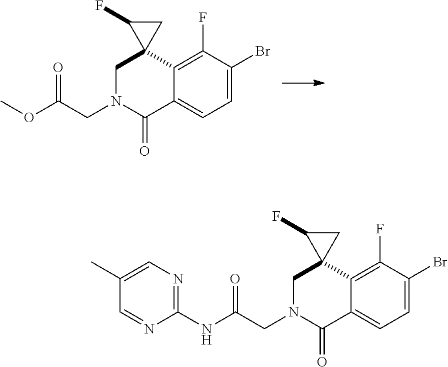

To a solution of methyl 2-[(2′s,4r)-6-bromo-2′,5-difluoro-1-oxo-spiro[3H-isoquinoline-4,1′-cyclopropane]-2-yl]acetate (50 mg, 0.14 mmol, Int. 43) in DCE (2 mL) was added 5-methylpyrimidin-2-amine (18 mg, 0.17 mmol) and AlMe₃ (1 M in n-heptane, 0.21 mL). The mixture was first stirred at 60° C. for 16 h and then 90° C. for 3 h. The reaction was quenched with H₂O (6 mL) and extracted with EtOAc (3×6 mL). The combined organic layers were washed with brine (6 mL), dried over anhydrous Na₂SO₄, filtered and concentrated under reduced pressure. The residue was purified by reverse-phase preparative HPLC. LCMS: m/z=436.9, 438.9 [M+H]⁺. ¹H NMR (400 MHz, DMSO-d₆): δ 10.71 (s, 1H), 8.51 (s, 2H), 7.81-7.66 (m, 2H), 5.68-5.39 (m, 1H), 4.72 (br d, J=17.2 Hz, 1H), 4.40 (br d, J=16.8 Hz, 1H), 3.84-3.74 (m, 1H), 3.72-3.62 (m, 1H), 2.26 (s, 3H), 1.90-1.75 (m, 1H), 1.73-1.58 (m, 1H).

### Example 226

### 2-[(2′s,4r)-6-bromo-2′,5-difluoro-1-oxospiro[3H-isoquinoline-4,1′-cyclopropane]-2-yl]-N-[5-(trifluoromethyl)pyrimidin-2-yl]acetamide (226)

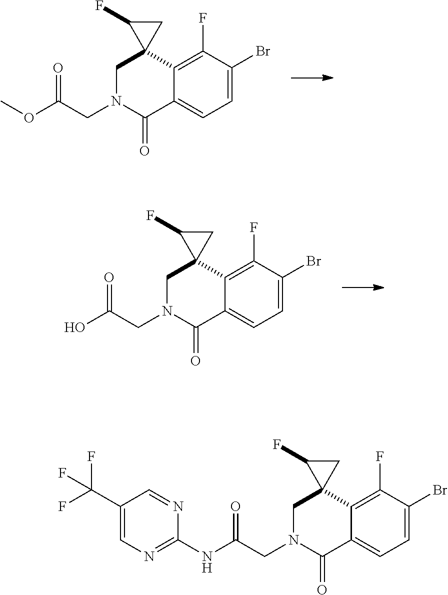

2-[(2′s,4r)-6-bromo-2′,5-difluoro-1-oxo-spiro[3H-isoquinoline-4,1′-cyclopropane]-2-yl]acetic acid: To a solution of methyl 2-[(2′s,4r)-6-bromo-2′,5-difluoro-1-oxo-spiro[3H-isoquinoline-4,1′-cyclopropane]-2-yl]acetate (500 mg, 1.39 mmol, Int. 43) in THF (5 mL) and H₂O (5 mL) was added LiOH·H₂O (146 mg, 3.47 mmol). The mixture was stirred for 1 h. The reaction mixture was diluted with H₂O (10 mL) and extracted with MTBE (10 mL). HCl (2M) was added to the aqueous phase to adjust the pH to 3-4 and the mixture was extracted with EtOAc (3×8 mL). The combined organic layers were washed with brine (10 mL), dried over anhydrous Na₂SO₄, filtered and concentrated under reduced pressure to provide a residue that was used directly. ¹H NMR (400 MHz, CDCl₃): δ 12.80 (br s, 1H), 7.73-7.22 (m, 2H), 5.70-5.38 (m, 1H), 4.38-4.06 (m, 2H), 3.81-3.59 (m, 2H), 1.89-1.77 (m, 1H), 1.70-1.54 (m, 1H).

2-[(2′s,4r)-6-bromo-2′,5-difluoro-1-oxospiro[3H-isoquinoline-4,1′-cyclopropane]-2-yl]-N-[5-(trifluoromethyl)pyrimidin-2-yl]acetamide: To a solution of 5-(trifluoromethyl)pyrimidin-2-amine (50 mg, 0.3 mmol) in THF (2 mL) at 0° C. was added LiHMDS (1 M in THF, 0.3 mL). The mixture was stirred at 0° C. for 1 h. To a solution of 2-[(2′s,4r)-6-bromo-2′,5-difluoro-1-oxo-spiro[3H-isoquinoline-4,1′-cyclopropane]-2-yl]acetic acid (70 mg, 0.2 mmol) in THF (2 mL) was added CDI (66 mg, 0.4 mmol). The latter mixture was stirred at 20° C. for 1 h and added to the former mixture at 0° C. The reaction was stirred at 20° C. for 16 h. The reaction mixture was diluted with H₂O (5 mL) and extracted with EtOAc (3×5 mL). The combined organic layers were washed with brine (5 mL), dried over anhydrous Na₂SO₄, filtered and concentrated under reduced pressure. The residue was purified by reverse-phase preparative HPLC. LCMS: m/z=490.9, 492.9 [M+H]⁺. ¹H NMR (400 MHz, CDCl₃): δ 8.83 (m, 3H), 7.90 (d, J=8.4 Hz, 1H), 7.56 (dd, J=6.4, 8.4 Hz, 1H), 5.45-5.18 (m, 1H), 5.02 (br d, J=17.2 Hz, 1H), 4.54 (d, J=17.2 Hz, 1H), 3.99-3.60 (m, 2H), 2.00-1.95 (m, 1H), 1.51-1.42 (m, 1H).

### Example 227

### 2-[(2′s,4r)-6-bromo-2′,5-difluoro-1-oxospiro[3H-isoquinoline-4,1′-cyclopropane]-2-yl]-N-pyrimidin-2-ylacetamide (227)

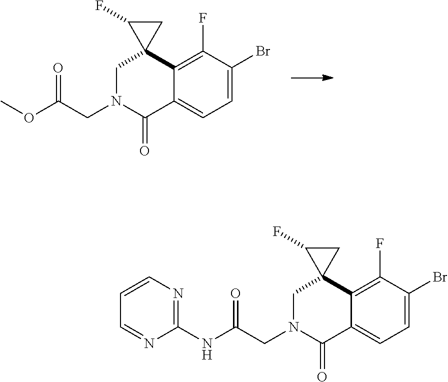

To a mixture of methyl 2-[(2′s,4r)-6-bromo-2′,5-difluoro-1-oxo-spiro[3H-isoquinoline-4,1′-cyclopropane]-2-yl]acetate (30.0 mg, 0.08 mmol. Int. 42) and pyrimidin-2-amine (16 mg, 0.16 mmol) in DCM (2 mL) was added AlMe₃ (1 M in n-heptane, 0.18 mL). The reaction mixture was stirred at 90° C. for 2 h. The reaction mixture was diluted with aq. sat. NH₄Cl (10 mL), and the resulting aqueous mixture was extracted with EtOAc (3×5 mL). The combined organic layers were washed with brine (5 mL), dried over anhydrous Na₂SO₄, filtered, and concentrated under reduced pressure. The residue was purified by reverse-phase preparative HPLC. LCMS: m/z=422.9, 424.9 [M+H]⁺. ¹H NMR (400 MHz, CDCl₃): δ 8.95 (br s, 1H), 8.62 (d, J=4.8 Hz, 2H), 7.90 (d, J=8.4 Hz, 1H), 7.55 (dd, J=6.8, 8.4 Hz, 1H), 7.04 (t, J=4.8 Hz, 1H), 5.50-5.20 (m, 1H), 5.09 (br d, J=16.8 Hz, 1H), 4.56 (br d, J=17.2 Hz, 1H), 3.91 (dd, J=2.4, 13.2 Hz, 1H), 3.69 (d, J=13.2 Hz, 1H), 1.96-1.90 (m, 1H), 1.57-1.43 (m, 1H).

### Example 228

### 2-[(2′s,4r)-6-cyclopropyl-2′,5-difluoro-1-oxospiro[3H-isoquinoline-4,1′-cyclopropane]-2-yl]-N-(5-fluoropyrimidin-2-yl)acetamide (228)

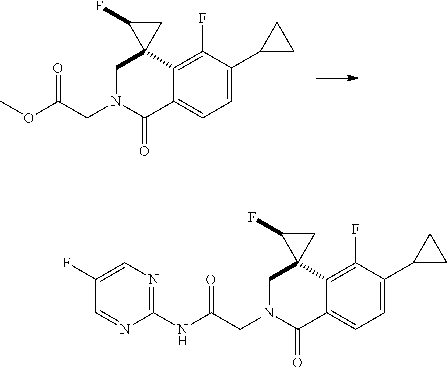

To a solution of methyl 2-[(2′s,4r)-6-cyclopropyl-2′,5-difluoro-1-oxo-spiro[3H-isoquinoline-4,1′-cyclopropane]-2-yl]acetate (50 mg, 0.16 mmol, Int. 44) and 5-fluoropyrimidin-2-amine (35 mg, 0.31 mmol) in DCE (1 mL) at 0° C. was added AlMe₃ (1 M in heptane, 0.314 mmol). The mixture was stirred at 90° C. for 3 h. The reaction mixture was quenched with H₂O (3 mL) and extracted with DCM:MeOH (V:V=10:1, 3×1 mL). The combined organic layers were washed with brine (1.5 mL), dried over anhydrous Na₂SO₄, filtered, concentrated under reduced pressure, and purified by reverse-phase preparative HPLC. LCMS: m/z=403.0 [M+H]⁺. ¹H NMR (400 MHz, CDCl₃): δ 8.92 (br s, 1H), 8.48 (s, 2H), 7.90 (d, J=8.4 Hz, 1H), 6.83 (t, J=7.6 Hz, 1H), 5.47-5.23 (m, 1H), 4.82 (br d, J=17.6 Hz, 1H), 4.41 (br d, J=16.0 Hz, 1H), 3.87 (d, J=13.2 Hz, 1H), 3.67 (d, J=13.2 Hz, 1H), 2.12-2.02 (m, 1H), 1.96-1.94 (m, 1H), 1.53-1.37 (m, 1H), 1.12-0.99 (m, 2H), 0.83-0.69 (m, 2H).

### Example 229

### 2-[(2′s,4r)-6-cyclopropyl-2′,5-difluoro-1-oxospiro[3H-isoquinoline-4,1′-cyclopropane]-2-yl]-N-(5-chloropyrimidin-2-yl)acetamide (229)

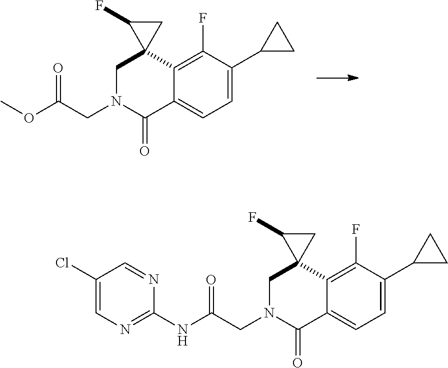

To a solution of methyl 2-[(2′s,4r)-6-cyclopropyl-2′,5-difluoro-1-oxo-spiro[3H-isoquinoline-4,1′-cyclopropane]-2-yl]acetate (130 mg, 0.40 mmol, Int. 44) and 5-chloropyrimidin-2-amine (104 mg, 0.81 mmol) in DCE (2 mL) was added AlMe₃ (1 M in heptane, 1.21 mL). The mixture was stirred at 60° C. for 12 h. The reaction mixture was quenched by addition of H₂O (8 mL) at 0° C., and extracted with EtOAc (3×5 mL). The combined organic layers were washed with brine (15 mL), dried over anhydrous Na₂SO₄, filtered, and concentrated under reduced pressure. The residue was purified by reverse-phase preparative HPLC. LCMS: m/z=419.0, 421.0 [M+H]⁺. ¹H NMR (400 MHz, CDCl₃) δ 8.90-8.79 (m, 1H), 8.55 (s, 2H), 7.90 (d, J=8.0 Hz, 1H), 6.83 (t, J=7.6 Hz, 1H), 5.45-5.25 (m, 1H), 4.84 (br d, J=16.8 Hz, 1H), 4.43 (br d, J=16.8 Hz, 1H), 3.87 (dd, J=2.4, 12.8 Hz, 1H), 3.67 (d, J=12.8 Hz, 1H), 2.11-2.03 (m, 1H), 1.99-1.92 (m, 1H), 1.49-1.40 (m, 1H), 1.08-1.02 (m, 2H), 0.79-0.73 (m, 2H).

### Example 230

### 2-[(2′s,4r)-6-cyclopropyl-2′,5-difluoro-1-oxospiro[3H-isoquinoline-4,1′-cyclopropane]-2-yl]-N-(5-cyanopyrimidin-2-yl)acetamide (230)

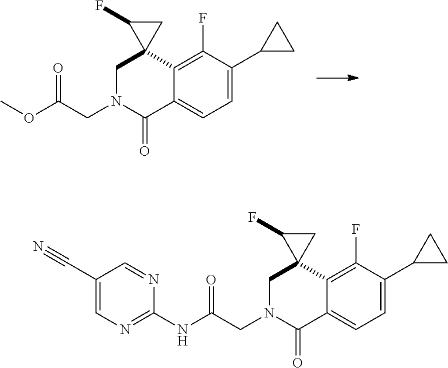

To a solution of methyl 2-[(2′s,4r)-6-cyclopropyl-2′,5-difluoro-1-oxo-spiro[3H-isoquinoline-4,1′-cyclopropane]-2-yl]acetate (130 mg, 0.40 mmol, Int. 44) and 2-aminopyrimidine-5-carbonitrile (97 mg, 0.81 mmol) in DCE (2 mL) was added AlMe₃ (1 M in heptane, 1.21 mL). The mixture was stirred at 80° C. for 2 h. The reaction mixture was quenched by addition of H₂O (8 mL) at 0° C., and extracted with EtOAc (3×5 mL). The combined organic layers were washed with brine (15 mL), dried over anhydrous Na₂SO₄, filtered, and concentrated under reduced pressure. The residue was purified by reverse-phase preparative HPLC. LCMS: m/z=410.0 [M+H]⁺. ¹H NMR (400 MHz, DMSO-d₆) δ 11.37 (br s, 1H), 9.13 (s, 2H), 7.68 (d, J=8.4 Hz, 1H), 6.97 (t, J=7.6 Hz, 1H), 5.64-5.43 (m, 1H), 4.75 (d, J=17.6 Hz, 1H), 4.46 (d, J=17.6 Hz, 1H), 3.78-3.71 (m, 1H), 3.68-3.62 (m, 1H), 2.09-1.97 (m, 1H), 1.87-1.75 (m, 1H), 1.68-1.55 (m, 1H), 1.09-0.94 (m, 2H), 0.85-0.68 (m, 2H).

### Example 231

### 2-[(2′s,4r)-6-cyclopropyl-2′-fluoro-1-oxospiro[3H-isoquinoline-4,1′-cyclopropane]-2-yl]-N-(5-fluoropyrimidin-2-yl)acetamide (231)

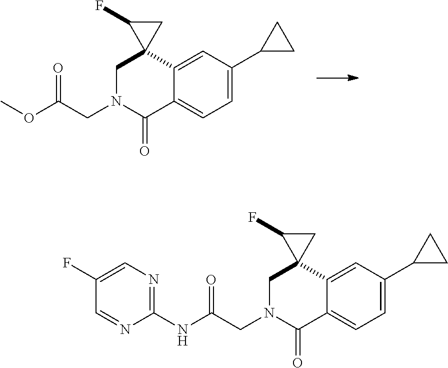

To a solution of methyl 2-[(2′s,4r)-6-cyclopropyl-2′-fluoro-1-oxo-spiro[3H-isoquinoline-4,1′-cyclopropane]-2-yl]acetate (25 mg, 0.08 mmol, Int. 45) and 5-fluoropyrimidin-2-amine (28 mg, 0.25 mmol) in DCE (1 mL) was added AlMe₃ (1 M in heptane, 0.25 mL). The mixture was stirred at 90° C. for 2 h. The mixture was quenched with H₂O (5 mL) and extracted with EtOAc (3×2 mL). The combined organic layers were washed with brine (2 mL), dried over anhydrous Na₂SO₄, filtered, concentrated under reduced pressure, and purified by. The residue was purified by reverse-phase preparative HPLC. LCMS: m/z=385.0 [M+H]⁺. ¹H NMR (400 MHz, CDCl₃): δ 9.00 (br s, 1H), 8.48 (s, 2H), 8.08 (d, J=8.0 Hz, 1H), 7.01 (dd, J=1.6, 8.0 Hz, 1H), 6.40 (s, 1H). 4.85-4.73 (m, 1H), 4.71-4.49 (m, 1H), 4.37 (d, J=16.0 Hz, 1H), 4.13 (dd, J=1.6, 12.8 Hz, 1H), 3.54 (d, J=12.8 Hz, 1H), 1.95-1.84 (m, 1H), 1.67-1.57 (m, 1H), 1.42-1.30 (m, 1H), 1.08-1.01 (m, 2H), 0.78-0.71 (m, 2H).

Example 231 has been identified as a single enantiomer, 2-[(2′R,4S)-6-cyclopropyl-2′-fluoro-1-oxospiro[3H-isoquinoline-4,1′-cyclopropane]-2-yl]-N-(5-fluoropyrimidin-2-yl)acetamide, as shown in Table 1A.

### Example 232

### 2-[(2′s,4r)-6-cyclopropyl-2′-fluoro-1-oxospiro[3H-isoquinoline-4,1′-cyclopropane]-2-yl]-N-(5-chloropyrimidin-2-yl)acetamide (232)

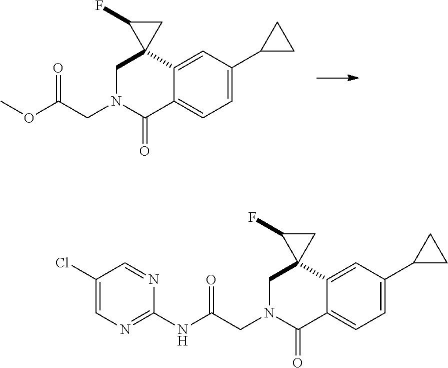

To a solution of methyl 2-[(2′s,4r)-6-cyclopropyl-2′-fluoro-1-oxo-spiro[3H-isoquinoline-4,1′-cyclopropane]-2-yl]acetate (50 mg, 0.16 mmol, Int. 45) and 5-chloropyrimidin-2-amine (42 mg, 0.33 mmol) in DCE (1 mL) was added AlMe₃ (1 M in heptane, 0.49 mL). The mixture was stirred at 60° C. for 12 h. The reaction mixture was quenched by addition of H₂O (5 mL) at 0° C., and extracted with EtOAc (3×2 mL). The combined organic layers were washed with brine (5 mL), dried over anhydrous Na₂SO₄, filtered, and concentrated under reduced pressure. The residue was purified by reverse-phase preparative HPLC. LCMS: m/z=401.0, 403.0 [M+H]⁺. ¹H NMR (400 MHz, DMSO-d₆) δ 10.99 (br s, 1H), 8.77 (s, 2H), 7.78 (d, J=8.0 Hz, 1H), 7.04 (d, J=8.0 Hz, 1H), 6.63 (s, 1H), 5.11-4.89 (m, 1H), 4.75 (br d, J=17.2 Hz, 1H), 4.28 (br d, J=16.8 Hz, 1H), 3.90 (br d, J=12.8 Hz, 1H), 3.52 (d, J=12.8 Hz, 1H), 1.98-1.90 (m, 1H), 1.72-1.63 (m, 1H), 1.49-1.39 (m, 1H), 1.02-0.96 (m, 2H), 0.80-0.74 (m, 2H).

Example 232 has been identified as a single enantiomer, N-(5-chloropyrimidin-2-yl)-2-[(2′R,4S)-6-cyclopropyl-2′-fluoro-1-oxospiro[3H-isoquinoline-4,1′-cyclopropane]-2-yl]acetamide, as shown in Table 1A.

### Example 233

### 2-[(2′s,4r)-6-cyclopropyl-2′-fluoro-1-oxospiro[3H-isoquinoline-4,1′-cyclopropane]-2-yl]-N-(5-cyanopyrimidin-2-yl)acetamide (233)

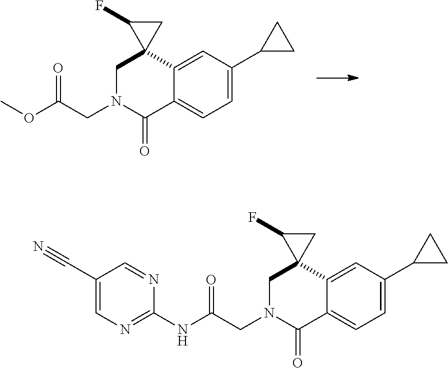

To a solution of methyl 2-[(2′s,4r)-6-cyclopropyl-2′-fluoro-1-oxo-spiro[3H-isoquinoline-4,1′-cyclopropane]-2-yl]acetate (50 mg, 0.16 mmol, Int. 45) and 2-aminopyrimidine-5-carbonitrile (39 mg, 0.32 mmol) in DCE (1 mL) was added AlMe₃ (1 M in heptane, 0.49 mL). The mixture was stirred at 80° C. for 2 h. The reaction mixture was quenched by addition of H₂O (2 mL) at 0° C., and then extracted with EtOAc (3×2 mL). The combined organic layers were washed with brine (5 mL), dried over anhydrous Na₂SO₄, filtered, and concentrated under reduced pressure to give a residue. The residue was purified by reverse-phase preparative HPLC. LCMS: m/z=392.0 [M+H]⁺. ¹H NMR (400 MHz, CDCl₃) δ 9.32 (br s, 1H), 8.85 (s, 2H), 8.07 (d, J=8.0 Hz, 1H), 7.02 (dd, J=1.6, 8.0 Hz, 1H), 6.41 (d, J=1.2 Hz, 1H), 4.81 (d, J=16.4 Hz, 1H), 4.70-4.49 (m, 1H), 4.41 (d, J=16.4 Hz, 1H), 4.14 (dd, J=2.0, 12.8 Hz, 1H), 3.52 (d, J=12.8 Hz, 1H), 1.97-1.85 (m, 1H), 1.65-1.62 (m, 1H), 1.41-1.29 (m, 1H), 1.10-1.01 (m, 2H), 0.78-0.70 (m, 2H).

Example 233 has been identified as a single enantiomer, N-(5-cyanopyrimidin-2-yl)-2-[(2′R,4S)-6-cyclopropyl-2′-fluoro-1-oxospiro[3H-isoquinoline-4,1′-cyclopropane]-2-yl]acetamide, as shown in Table 1A.

### Example 234 and 235

### 2-[(2′s,4r)-2′-fluoro-1-oxo-6-(trifluoromethyl)spiro[3H-isoquinoline-4,1′-cyclopropane]-2-yl]-N-(5-fluoropyrimidin-2-yl)acetamide (234 and 235)

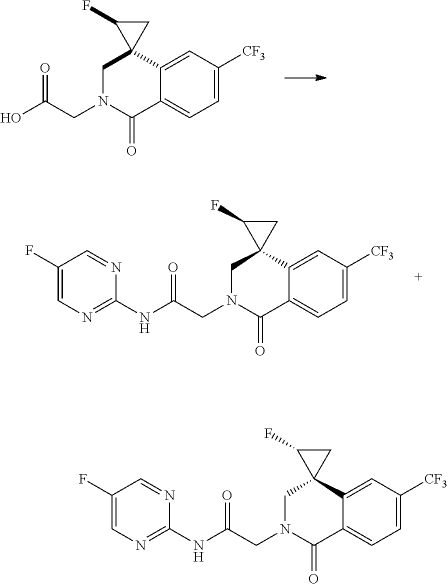

To a solution of 2-[(2′s,4r)-2′-fluoro-1-oxo-6-(trifluoromethyl)spiro[3H-isoquinoline-4,1′-cyclopropane]-2-yl]acetic acid (35 mg, 0.11 mmol, Int. 49) in pyridine (1.0 mL) was added 5-fluoropyrimidin-2-amine (31 mg, 0.28 mmol) and EDCI (42 mg, 0.22 mmol). The mixture was stirred for 2 h. The mixture was diluted with H₂O (5 mL) and extracted with EtOAc (3×2 mL). The combined organic layers were washed with brine (3 mL), dried over anhydrous Na₂SO₄, filtered, and concentrated under reduced pressure. The residue was purified by reverse-phase preparative HPLC. The mixture was further purified by chiral SFC (Column: Daicel Chiralpak AD (250 mm×30 mm, 10 μm particle size); Mobile phase: A: CO₂ and B: 0.1% NH₄OH in i-PrOH; B %: 35% isocratic, Flow rate: 3.4 mL/min; Detection Wavelength: 220 nm Column Temperature: 40° C.; System back pressure: 124 bar) to provide:

2-[(2′s,4r)-2′-fluoro-1-oxo-6-(trifluoromethyl)spiro[3H-isoquinoline-4,1′-cyclopropane]-2-yl]-N-(5-fluoropyrimidin-2-yl)acetamide (first eluting isomer, 234): LCMS: m/z=413.0 [M+H]⁺. ¹H NMR (400 MHz, CDCl₃): δ 8.75 (br s, 1H), 8.49 (s, 2H), 8.32 (d, J=8.4 Hz, 1H), 7.65 (d, J=8.0 Hz, 1H), 6.95 (s, 1H), 5.13-4.94 (m, 1H), 4.77-4.54 (m, 1H), 4.42 (br d, J=16.8 Hz, 1H), 4.30-4.19 (m, 1H), 3.57 (d, J=12.8 Hz, 1H), 1.75-1.65 (m, 1H), 1.53-1.42 (m, 1H).

2-[(2′s,4r)-2′-fluoro-1-oxo-6-(trifluoromethyl)spiro[3H-isoquinoline-4,1′-cyclopropane]-2-yl]-N-(5-fluoropyrimidin-2-yl)acetamide (second eluting isomer, 235) was further purified by reverse-phase preparative HPLC: LCMS: m/z=413.0 [M+H]⁺. ¹H NMR (400 MHz, DMSO-d₆): δ 10.99 (s, 1H), 8.77 (s, 2H), 8.12 (d, J=8.0 Hz, 1H), 7.75 (d, J=8.0 Hz, 1H), 7.37 (s, 1H), 5.27-5.05 (m, 1H), 4.78 (br d, J=16.8 Hz, 1H), 4.35 (d, J=17.2 Hz, 1H), 3.97 (dd, J=1.6, 13.2 Hz, 1H), 3.61 (d, J=12.8 Hz, 1H), 1.78-1.88 (m, 1H), 1.62-1.51 (m, 1H).

### Example 236 and 237

### N-(5-cyanopyrimidin-2-yl)-2-[(2′s,4r)-2′-fluoro-1-oxo-6-(trifluoromethyl)spiro[3H-isoquinoline-4,1′-cyclopropane]-2-yl]acetamide (236 and 237)

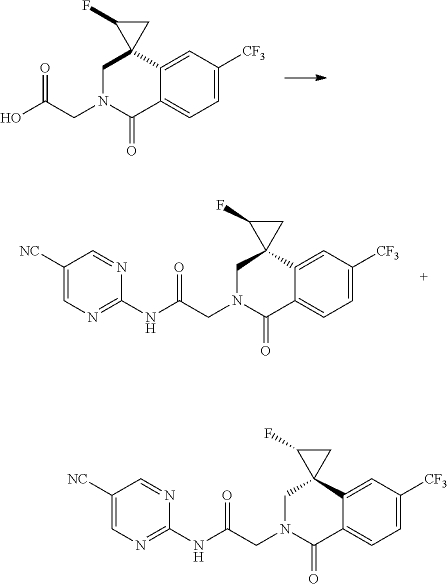

To a solution of 2-aminopyrimidine-5-carbonitrile (280 mg, 2.36 mmol) in THF (5 mL) was added CDI (99 mg, 0.6 mmol). The mixture was stirred for 1 h and cooled to 0° C. To the mixture was added a solution of 2-[(2′s,4r)-2′-fluoro-1-oxo-6-(trifluoromethyl)spiro[3H-isoquinoline-4,1′-cyclopropane]-2-yl]acetic acid (500 mg, 1.58 mmol, Int. 49) in THF (2 mL) followed by LiHMDS (1 M in n-heptane, 0.4 mL). The reaction mixture was stirred at 0° C. for 0.5 h and then stirred at 25° C. for a further 2 h. The reaction mixture was quenched by addition of water (20 mL) at 0° C., and then extracted with EtOAc (3×20 mL). The combined organic layers were washed with brine (25 mL), dried over anhydrous Na₂SO₄, filtered, and concentrated under reduced pressure. The residue was purified by reverse-phase preparative HPLC. The mixture was further purified by chiral SFC (Column: Daicel Chiralpak IC (250 mm×30 mm, 10 μm particle size); Mobile phase: A: CO₂ and B: 0.1% NH₄OH in i-PrOH; B %: 60% isocratic, Flow rate: 4 mL/min; Column Temperature: 35° C.; System back pressure: 124 bar) to provide:

N-(5-cyanopyrimidin-2-yl)-2-[(2′s,4r)-2′-fluoro-1-oxo-6-(trifluoromethyl)spiro[3H-isoquinoline-4,1′-cyclopropane]-2-yl]acetamide (first eluting isomer, 236): LCMS: m/z=420.0 [M+H]⁺. ¹H NMR (400 MHz, DMSO-d₆): δ11.40 (br s, 1H), 9.11 (s, 2H), 8.11 (d, J=8.0 Hz, 1H), 7.74 (d, J=8.0 Hz, 1H), 7.35 (s, 1H), 5.29-4.98 (m, 1H), 4.84 (d, J=17.2 Hz, 1H), 4.43 (d, J=17.2 Hz, 1H), 3.95 (dd, J=1.6, 13.0 Hz, 1H), 3.61 (s, 1H), 1.83 (td, J=7.2, 12.1 Hz, 1H), 1.64-1.44 (m, 1H).

N-(5-cyanopyrimidin-2-yl)-2-[(2′s,4r)-2′-fluoro-1-oxo-6-(trifluoromethyl)spiro[3H-isoquinoline-4,1′-cyclopropane]-2-yl]acetamide (second eluting isomer, 237): LCMS: m/z=420.0 [M+H]⁺. ¹H NMR (400 MHz, DMSO-d₆): δ11.42 (br s, 1H), 9.12 (s, 2H), 8.12 (d, J=8.0 Hz, 1H), 7.75 (dd, J=0.8, 8.0 Hz, 1H), 7.36 (s, 1H), 5.27-5.02 (m, 1H), 4.85 (d, J=17.2 Hz, 1H), 4.44 (d, J=17.2 Hz, 1H), 3.96 (dd, J=2.0, 13.2 Hz, 1H), 3.60 (d, J=13.2 Hz, 1H), 1.84 (td, J=7.2, 12.0 Hz, 1H), 1.63-1.48 (m, 1H).

### Example 238 and 239

### N-(5-chloropyrimidin-2-yl)-2-[(2′s,4r)-2′-fluoro-1-oxo-6-(trifluoromethyl)spiro[3H-isoquinoline-4,1′-cyclopropane]-2-yl]acetamide (238 and 239)

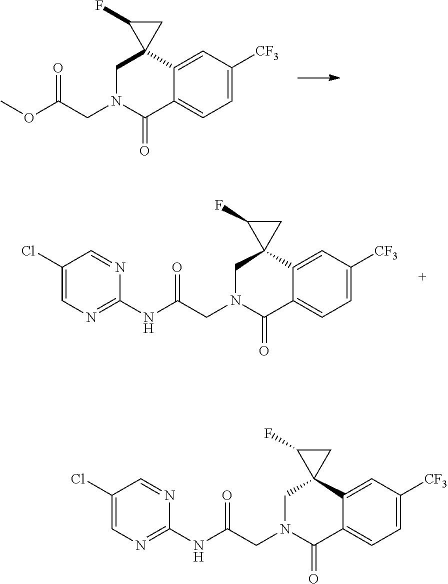

To a solution of methyl 2-[(2′s,4r)-2′-fluoro-1-oxo-6-(trifluoromethyl)spiro[3H-isoquinoline-4,1′-cyclopropane]-2-yl]acetate (150 mg, 0.45 mmol, Int. 48) and 5-chloropyrimidin-2-amine (176 mg, 1.36 mmol) in DCE (2.0 mL) was added AlMe₃ (1 M in heptane, 1.36 mL). The mixture was stirred at 60° C. for 5 h. The mixture was diluted with H₂O (1 mL) and extracted with DCM (3×1 mL). The combined organic layers were washed with brine (1 mL), dried over anhydrous Na₂SO₄, filtered, and concentrated under reduced pressure. The residue was purified by reverse-phase preparative HPLC. The mixture was further purified by chiral SFC (Column: Chiralpak WK-3, (100 mm×4.6 mm, 3 m particle size); Mobile phase: A: CO₂ and B: 0.1% EtOH in i-PrOH); Gradient: B %=50% isocratic; Flow rate 3.4 mL/min; Column temperature: 35° C. System back pressure: 137 bar) to provide:

N-(5-chloropyrimidin-2-yl)-2-[(2′s,4r)-2′-fluoro-1-oxo-6-(trifluoromethyl)spiro[3H-isoquinoline-4,1′-cyclopropane]-2-yl]acetamide (first eluting isomer, 238): LCMS: m/z=429.1, 431.1 [M+H]⁺. ¹H NMR (400 MHz, CDCl₃): δ 8.94 (br s, 1H), 8.56 (s, 2H), 8.32 (d, J=8.0 Hz, 1H), 7.65 (d, J=8.0 Hz, 1H), 6.95 (s, 1H), 5.09 (br d, J=16.4 Hz, 1H), 4.82-4.54 (m, 1H), 4.44 (d, J=17.2 Hz, 1H), 4.23 (dd, J=2.0, 12.8 Hz, 1H), 3.56 (d, J=12.9 Hz, 1H), 1.72-1.66 (m, 1H), 1.53-1.41 (m, 1H).

N-(5-chloropyrimidin-2-yl)-2-[(2′s,4r)-2′-fluoro-1-oxo-6-(trifluoromethyl)spiro[3H-isoquinoline-4,1′-cyclopropane]-2-yl]acetamide (second eluting isomer, 239): LCMS: m/z=429.1, 431.1 [M+H]⁺. H NMR (400 MHz, CDCl₃): δ 8.79 (br s, 1H), 8.56 (s, 2H), 8.32 (d, J=8.4 Hz, 1H), 7.65 (d, J=8.0 Hz, 1H), 6.95 (s, 1H), 5.07 (br d, J=17.2 Hz, 1H), 4.77-4.55 (m, 1H), 4.43 (d, J=17.2 Hz, 1H), 4.23 (dd, J=12.8, 12.4 Hz, 1H), 3.56 (d, J=12.8 Hz, 1H), 1.70-1.67 (m, 1H), 1.50-1.41 (m, 1H).

### Example 240 and 241

### 2-[(2′s,4r)-2′-fluoro-1-oxo-6-(trifluoromethyl)spiro[3H-isoquinoline-4,1′-cyclopropane]-2-yl]-N-pyrimidin-2-ylacetamide (240 and 241)

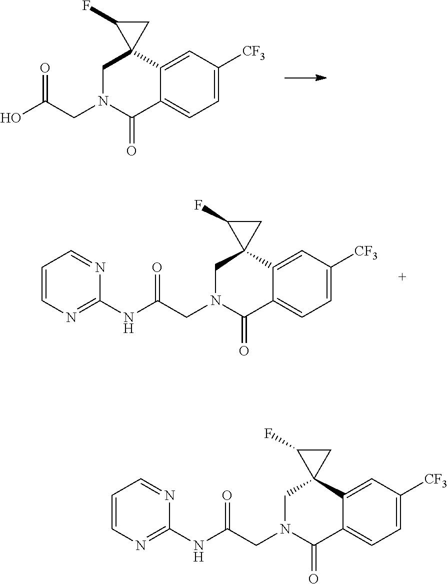

To a solution of 2-[(2′s,4r)-2′-fluoro-1-oxo-6-(trifluoromethyl)spiro[3H-isoquinoline-4,1′-cyclopropane]-2-yl]acetic acid (80 mg, 0.25 mmol, Int. 49) in pyridine (1.0 mL) was added pyrimidin-2-amine (60 mg, 0.63 mmol) and EDCI (97 mg, 0.51 mmol). The mixture was stirred for 3 h. The mixture was diluted with H₂O (4 mL) and extracted with EtOAc (3×2 mL). The combined organic layers were washed with brine (3 mL), dried over anhydrous Na₂SO₄, filtered and concentrated under reduced pressure. The residue was purified by reverse-phase preparative HPLC. The mixture was further purified by chiral SFC (Column: Regis(S,S)Whelk-O1 (250 mm×25 mm, 10 μm particle size); Mobile phase: A: CO₂ and B: 0.1% NH₄OH in i-PrOH; Gradient: 50% B isocratic; Flow rate: 3.4 mL/min; Detection wavelength: 220 nm; Column temperature: 40° C.; System back pressure: 137 bar) to provide:

2-[(2′s,4r)-2′-fluoro-1-oxo-6-(trifluoromethyl)spiro[3H-isoquinoline-4,1′-cyclopropane]-2-yl]-N-pyrimidin-2-ylacetamide (first eluting isomer, 240): LCMS: m/z=395.0 [M+H]⁺. ¹H NMR (400 MHz, CDCl₂): δ 8.86 (br s, 1H), 8.62 (br d, J=4.4 Hz, 2H), 8.32 (d, J=8.0 Hz, 1H), 7.64 (br d, J=8.0 Hz, 1H), 7.04 (br t, J=4.4 Hz, 1H), 6.95 (s, 1H), 5.21 (br d, J=17.2 Hz, 1H), 4.79-4.56 (m, 1H), 4.50 (br d, J=17.2 Hz, 1H), 4.24 (br d, J=12.8 Hz, 1H), 3.57 (d, J=12.8 Hz, 1H), 1.71-1.64 (m, 1H), 1.54-1.41 (m, 1H).

2-[(2′s,4r)-2′-fluoro-1-oxo-6-(trifluoromethyl)spiro[3H-isoquinoline-4,1′-cyclopropane]-2-yl]-N-pyrimidin-2-ylacetamide (second eluting isomer, 241): LCMS: m/z=395.0 [M+H]⁺. ¹H NMR (400 MHz, CDCl₂): δ 9.00 (br s, 1H), 8.63 (br d, J=4.0 Hz, 2H), 8.32 (br d, J=8.0 Hz, 1H), 7.64 (br d, J=8.0 Hz, 1H), 7.04 (br s, 1H), 6.94 (br s, 1H), 5.22 (br d, J=16.4 Hz, 1H), 4.78-4.56 (m, 1H), 4.50 (br d, J=17.2 Hz, 1H), 4.23 (br d, J=12.0 Hz, 1H), 3.56 (br d, J=12.8 Hz, 1H), 1.70-1.63 (m, 1H), 1.53-1.41 (m, 1H).

### Example 242 and 243

### 2-[(2′s,4r)-2′-fluoro-1-oxo-6-(trifluoromethyl)spiro[3H-isoquinoline-4,1′-cyclopropane]-2-yl]-N-[5-(trifluoromethyl)pyrimidin-2-yl]acetamide (242 and 243)

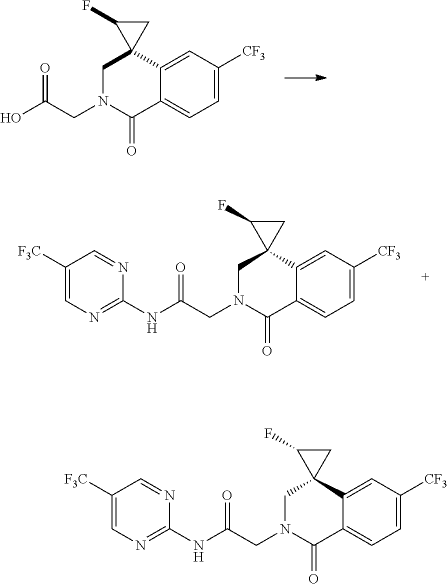

To a solution of 5-(trifluoromethyl)pyrimidin-2-amine (77 mg, 0.47 mmol) in THF (1 mL) at 0° C. was added LiHMDS (1 M in THF, 0.44 mL). The mixture was stirred at 0° C. for 1 h. Separately, a solution of 2-[(2′s,4r)-2′-fluoro-1-oxo-6-(trifluoromethyl)spiro[3H-isoquinoline-4,1′-cyclopropane]-2-yl]acetic acid (100 mg, 0.32 mmol, Int. 49) in THF (1 mL) was added CDI (102 mg, 0.63 mmol) was stirred at 20° C. for 1 h and then added to the first mixture at 0° C. The combined reaction mixture was stirred at 20° C. for 5 h. The mixture was diluted with H₂O (50 mL) and extracted with EtOAc (3×20 mL). The combined organic layers were washed with brine (30 mL), dried over anhydrous Na₂SO₄, filtered and concentrated under reduced pressure. The residue was purified by reverse-phase preparative HPLC. The mixture was further purified by chiral SFC (Column: (S,S)-Whelk-O1, (50 mm×4.6 mm, 3.5 m particle size); Mobile phase: A: CO₂ and B: 0.1% i-PrNH₂ in i-PrOH; Gradient: 41% B isocratic; Detection wavelength: 220 nm; How rate: 3.4 mL/min; Column temperature: 35° C.; System back pressure: 124 bar) to provide:

2-[(2′s,4r)-2′-fluoro-1-oxo-6-(trifluoromethyl)spiro[3H-isoquinoline-4,1′-cyclopropane]-2-yl]-N-[5-(trifluoromethyl)pyrimidin-2-yl]acetamide (first eluting isomer, 242): LCMS: m/z=463.1 [M+H]⁺. ¹H NMR (400 MHz, CDCl₃): δ 9.02 (br s, 1H), 8.85 (s, 2H), 8.32 (d, J=8.4 Hz, 1H), 7.65 (d, J=8.8 Hz, 1H), 6.95 (s, 1H), 5.17 (br d, J=18.4 Hz, 1H), 4.78-4.55 (m, 1H), 4.49 (d, J=18.4 Hz, 1H), 4.30-4.20 (m, 1H), 3.56 (d, J=12.4 Hz, 1H), 1.76-1.65 (m, 1H), 1.53-1.41 (m, 1H).

2-[(2′s,4r)-2′-fluoro-1-oxo-6-(trifluoromethyl)spiro[3H-isoquinoline-4,1′-cyclopropane]-2-yl]-N-[5-(trifluoromethyl)pyrimidin-2-yl]acetamide (second eluting isomer, 243): LCMS: m/z=463.1 [M+H]⁺. ¹H NMR (400 MHz, CDCl₃): δ 9.03 (br s, 1H), 8.85 (s, 2H), 8.32 (d, J=8.4 Hz, 1H), 7.65 (d, J=8.8 Hz, 1H), 6.95 (s, 1H), 5.17 (br d, J=18.0 Hz, 1H), 4.78-4.55 (m, 1H), 4.49 (d, J=18.0 Hz, 1H), 4.30-4.20 (m, 1H), 3.56 (d, J=12.8 Hz, 1H), 1.76-1.65 (m, 1H), 1.53-1.41 (m, 1H).

### Example 244 and 245

### 2-[(2′s,4r)-2′-fluoro-1-oxo-6-(trifluoromethyl)spiro[3H-isoquinoline-4,1′-cyclopropane]-2-yl]-N-(5-methylpyrimidin-2-yl)acetamide (244 and 245)

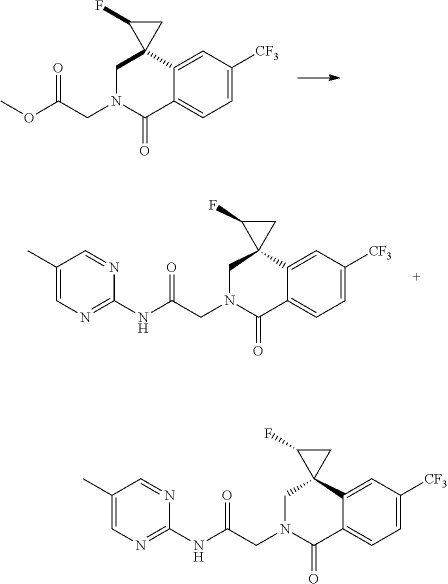

To a solution of methyl 2-[(2′s,4r)-2′-fluoro-1-oxo-6-(trifluoromethyl)spiro[3H-isoquinoline-4,1′-cyclopropane]-2-yl]acetate (100 mg, 0.30 mmol, Int. 48) in DCE (2 mL) were added 5-methylpyrimidin-2-amine (40 mg, 0.62 mmol) and AlMe₃ (1 M in n-heptane, 0.46 mL). The mixture was stirred at 60° C. for 16 h. The reaction mixture was quenched by addition of H₂O (15 mL) at 0° C. and extracted with EtOAc (3×10 mL). The combined organic layers were filtered and concentrated under reduced pressure. The residue was purified by reverse-phase preparative HPLC. The mixture was further purified by chiral SFC (Column: Daicel Chiralpak AD (250 mm×30 mm, 10 μm particle size); mobile phase: A: CO₂ and B: 0.1% NH₃H₂O in i-PrOH; Gradient: 40% B isocratic); Detection wavelength: 220 nm; Flow rate: 3.4 mL/min; Column temperature: 35° C.; System back pressure: 124 bar) to provide:

2-[(2′s,4r)-2′-fluoro-1-oxo-6-(trifluoromethyl)spiro[3H-isoquinoline-4,1′-cyclopropane]-2-yl]-N-(5-methylpyrimidin-2-yl)acetamide (first eluting isomer, 244): LCMS: m/z=409.0 [M+H]⁺. ¹H NMR (400 MHz, CDCl₃): δ 9.09 (s, 1H), 8.45 (s, 2H), 8.31 (d, J=8.12 Hz, 1H), 7.64 (d, J=8.00 Hz, 1H), 6.94 (s, 1H), 5.26-5.13 (m, 1H), 4.77-4.55 (m, 1H), 4.48 (br d, J=17.40 Hz, 1H), 4.23 (dd, J=12.88, 1.40 Hz, 1H), 3.56 (d, J=12.76 Hz, 1H), 2.28 (s, 3H), 1.71-1.65 (m, 1H), 1.53-1.40 (m, 1H).

2-[(2′s,4r)-2′-fluoro-1-oxo-6-(trifluoromethyl)spiro[3H-isoquinoline-4,1′-cyclopropane]-2-yl]-N-(5-methylpyrimidin-2-yl)acetamide (second eluting isomer, 245): LCMS: m/z=409.0 [M+H]⁺. ¹H NMR (400 MHz, CDCl₃): δ 9.16 (br s, 1H), 8.45 (s, 2H), 8.31 (d, J=8.00 Hz, 1H), 7.63 (br d, J=8.00 Hz, 1H), 6.94 (s, 1H), 5.22-5.18 (m, 1H), 4.78-4.56 (m, 1H), 4.48 (br d, J=17.24 Hz, 1H), 4.23 (br d, J=12.76 Hz, 1H), 3.56 (d, J=12.88 Hz, 1H), 2.28 (s, 3H), 1.70-1.63 (m, 1H), 1.53-1.41 (m, 1H).

### Example 246

### 2-[(2′s,4r)-2′-fluoro-6-iodo-1-oxospiro[3H-isoquinoline-4,1′-cyclopropane]-2-yl]-N-(5-fluoropyrimidin-2-yl)acetamide (246)

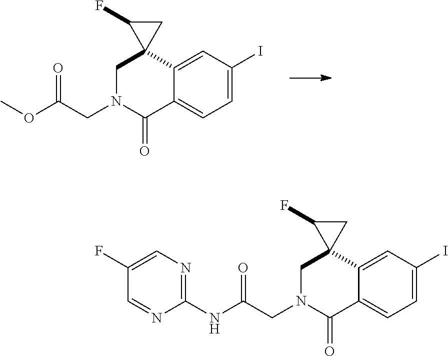

To a solution of methyl 2-[(2′s,4r)-2′-fluoro-6-iodo-1-oxo-spiro[3H-isoquinoline-4,1′-cyclopropane]-2-yl]acetate (50 mg, 0.13 mmol, Int. 50) and 5-fluoropyrimidin-2-amine (44 mg, 0.39 mmol) in DCE (2.0 mL) was added AlMe₃ (1 M in heptane, 0.39 mL). The mixture was stirred at 90° C. for 1.5 h. The reaction mixture was diluted with H₂O (15 mL), filtered, and extracted with EtOAc (3×10 mL). The combined organic layers were dried over anhydrous Na₂SO₄, filtered, concentrated under reduced pressure, and purified by reverse-phase preparative HPLC. LCMS: m/z: 471.0 [M+H]⁺. ¹H NMR (400 MHz, CDCl₃): δ 8.77 (br s, 1H), 8.48 (s, 2H), 7.89 (d, J=8.4 Hz, 1H), 7.75 (dd, J=8.4, 1.6 Hz, 1H), 7.05 (d, J=1.2 Hz, 1H), 4.99-4.83 (m, 1H), 4.72-4.50 (m, 1H), 4.37 (br d, J=16.4 Hz, 1H), 4.20-4.12 (m, 1H), 3.55-3.47 (m, 1H), 1.64-1.59 (m, 1H), 1.45-1.35 (m, 1H).

Example 246 has been identified as a single enantiomer, 2-[(2′R,4S)-2′-fluoro-6-iodo-1-oxospiro[3H-isoquinoline-4,1′-cyclopropane]-2-yl]-N-(5-fluoropyrimidin-2-yl)acetamide, as shown in Table 1A.

### Example 247

### 2-[(2′s,4r)-2′-fluoro-6-iodo-1-oxospiro[3H-isoquinoline-4,1′-cyclopropane]-2-yl]-N-pyrimidin-2-ylacetamide (247)

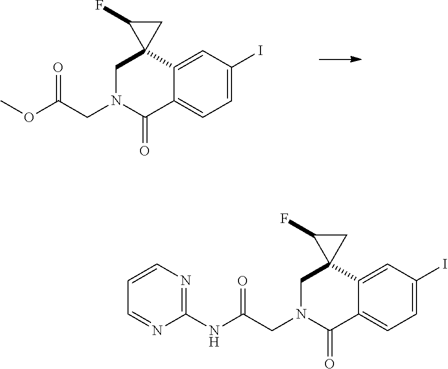

To a solution of methyl 2-[(2′s,4r)-2′-fluoro-6-iodo-1-oxo-spiro[3H-isoquinoline-4,1′-cyclopropane]-2-yl]acetate (50 mg, 0.13 mmol, Int. 50) and pyrimidin-2-amine (37 mg, 0.39 mmol) in DCE (2 mL) was added AlMe₃ (1 M in heptane, 0.39 mL). The mixture was stirred at 90° C. for 1.5 h. The mixture was diluted with H₂O (15 mL), filtered, and extracted with EtOAc (3×10 mL). The combined organic layers were dried over anhydrous Na₂SO₄, filtered, concentrated under reduced pressure, and purified by reverse-phase preparative HPLC. LCMS: m/z=453.0 [M+H]⁺. ¹H NMR (400 MHz, CDCl₃): δ 8.76 (br s, 1H), 8.61-8.55 (m, 2H), 7.89 (d, J=8.2 Hz, 1H), 7.77-7.70 (m, 1H), 7.09-6.98 (m, 2H), 5.17-5.01 (m, TH), 4.74-4.50 (m, 1H), 4.48-4.38 (m, 1H), 4.18 (br d, J=12.8 Hz, 1H), 3.51 (br d, J=12.8 Hz, 1H), 1.65-1.60 (m, 1H), 1.46-1.35 (m, 1H).

Example 247 has been identified as a single enantiomer, 2-[(2′R,4S)-2′-fluoro-6-iodo-1-oxospiro[3H-isoquinoline-4,1′-cyclopropane]-2-yl]-N-pyrimidin-2-ylacetamide, as shown in Table 1A.

### Example 248

### 2-[(2′s,4r)-2′-fluoro-6-iodo-1-oxospiro[3H-isoquinoline-4,1′-cyclopropane]-2-yl]-N-(3-hydroxy-3-methylcyclobutyl)acetamide (248)

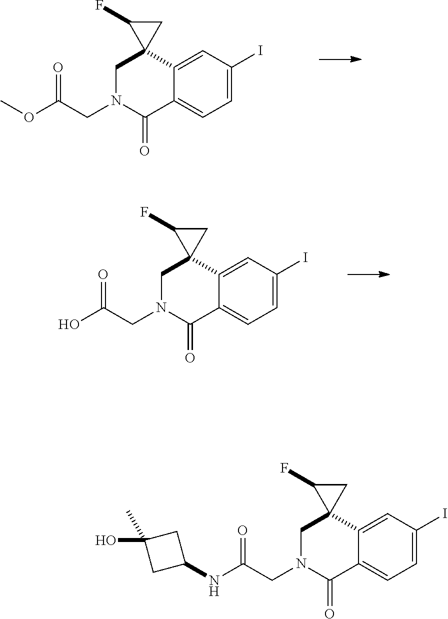

2-[(2′s,4r)-2′-fluoro-6-iodo-1-oxo-spiro[3H-isoquinoline-4,1′-cyclopropane]-2-yl]acetic acid: To a solution of methyl 2-[(2′s,4r)-2′-fluoro-6-iodo-1-oxo-spiro[3H-isoquinoline-4,1′-cyclopropane]-2-yl]acetate (160 mg, 0.4 mmol, Int. 50) in THF (2.0 mL) and H₂O (2.0 mL) was added LiOH·H₂O (43 mg, 1.03 mmol). The mixture was stirred for 3 h. The mixture was poured into H₂O (8 mL) and washed with MTBE (5 mL). The aqueous layer was adjusted to pH=3 with HCl (2 M) and extracted with EtOAc (3×3 mL). The combined organic layers were washed with brine (3 mL), dried over anhydrous Na₂SO₄, filtered, and concentrated under reduced pressure to provide a residue that was used directly. LCMS: m/z=376.0 [M+H]⁺.

2-[(2′s,4r)-2′-fluoro-6-iodo-1-oxospiro[3H-isoquinoline-4,1′-cyclopropane]-2-yl]-N-(cis-3-hydroxy-3-methylcyclobutyl)acetamide: To a solution of 2-[(2′s,4r)-2′-fluoro-6-iodo-1-oxo-spiro[3H-isoquinoline-4,1′-cyclopropane]-2-yl]acetic acid (235 mg, 0.63 mmol) in DMF (3.0 mL) were added cis-3-amino-1-methylcyclobutanol HCl salt (103 mg, 0.75 mmol), DIPEA (243 mg, 1.88 mmol), HOBt (127 mg, 0.94 mmol), and EDCI (180 mg, 0.94 mmol). The mixture was stirred for 3 h. The mixture was poured into H₂O (8 mL) and extracted with EtOAc (3×3 mL). The combined organic layers were washed with brine (3 mL), dried over anhydrous Na₂SO₄, filtered, concentrated under reduced pressure, and purified by reverse-phase preparative HPLC. LCMS: m/z=459.0 [M+H]⁺. ¹H NMR (400 MHz, CDCl₃): δ 7.86 (d, J=8.0 Hz, 1H), 7.76 (dd, J=1.2, 8.0 Hz, 1H), 7.06 (s, 1H), 6.44 (d, J=7.2 Hz, 1H), 4.68-4.44 (m, 1H), 4.34 (d, J=16.0 Hz, 1H), 4.11-3.93 (m, 3H), 3.44 (d, J=13.2 Hz, 1H), 2.58-2.45 (m, 2H), 2.08-1.96 (m, 3H), 1.72-1.63 (m, 1H), 1.45-1.33 (m, 4H).

Example 248 has been identified as a single enantiomer, 2-[(2′R,4S)-2′-fluoro-6-iodo-1-oxospiro[3H-isoquinoline-4,1′-cyclopropane]-2-yl]-N-(cis-3-hydroxy-3-methylcyclobutyl)acetamide, as shown in Table 1A.

### Example 249

### 2-[(2′s,4r)-2′-fluoro-6-(1-fluorocyclopropyl)-1-oxospiro[3H-isoquinoline-4,1′-cyclopropane]-2-yl]-N-(5-fluoropyrimidin-2-yl)acetamide (249)

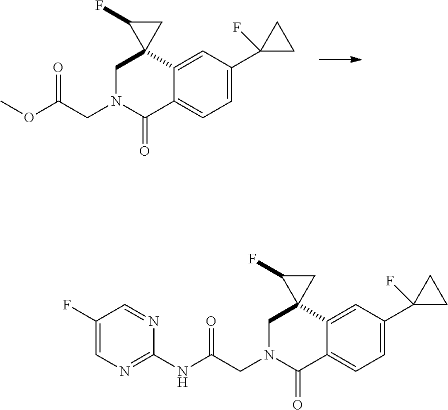

To a mixture of methyl 2-[(2′s,4r)-2′-fluoro-1-oxo-6-(1-fluorocyclopropyl)spiro[3H-isoquinoline-4,1′-cyclopropane]-2-yl]acetate (60 mg, 0.19 mmol, Int. 51), 5-fluoropyrimidin-2-amine (42 mg, 0.37 mmol) in DCE (2.0 mL) was added AlMe₃ (1 M in n-heptane, 0.56 mL). The mixture was stirred for 10 h at 60° C. The reaction mixture was poured into ice-cold H₂O (15 mL) and extracted with EtOAc (4×10 mL). The combined organic layers were washed with brine (2×10 mL), dried over anhydrous Na₂SO₄, filtered and concentrated under reduced pressure. The residue was purified by reverse-phase preparative HPLC. LCMS: m/z=403.0 [M+H]⁺. ¹H NMR (400 MHz, CDCl₃): δ 8.95 (br s, 1H), 8.48 (s, 2H), 8.16 (d, J=8.0 Hz, 1H). 7.02 (br d, J=7.6 Hz, 1H), 6.73 (s, 1H), 4.88 (br d, J=14.4 Hz, 1H), 4.76-4.51 (m, 1H), 4.39 (br d, J=16.4 Hz, 1H), 4.25-4.13 (m, 1H), 3.56 (d, J=12.8 Hz, 1H), 1.72-1.59 (m, 2H), 1.56-1.51 (m, 1H), 1.46-1.34 (m, 1H), 1.17-1.08 (m, 2H).

Example 249 has been identified as a single enantiomer, 2-[(2′R,4S)-2′-fluoro-6-(1-fluorocyclopropyl)-1-oxospiro[3H-isoquinoline-4,1′-cyclopropane]-2-yl]-N-(5-fluoropyrimidin-2-yl)acetamide, as shown in Table 1A.

### Example 250

### N-(5-chloropyrimidin-2-yl)-2-[(2′s,4r)-2′-fluoro-6-(1-fluorocyclopropyl)-1-oxospiro[3H-isoquinoline-4,1′-cyclopropane]-2-yl]acetamide (250)

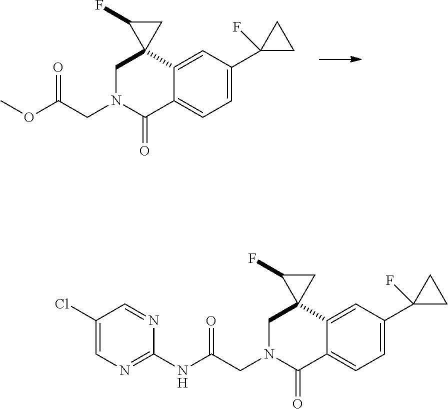

To a mixture of methyl 2-[(2′s,4r)-2′-fluoro-1-oxo-6-(1-fluorocyclopropyl)spiro[3H-isoquinoline-4,1′-cyclopropane]-2-yl]acetate (20 mg, 0.06 mmol, Int. 51), 5-chloropyrimidin-2-amine (16 mg, 0.12 mmol) in DCE (2 mL) was added AlMe₃ (1 M in n-heptane, 0.18 mL). The mixture was stirred for 12 h at 60° C. The reaction mixture was poured into H₂O (15 mL) at 0° C., and extracted with EtOAc (4×10 mL). The combined organic layers were washed with brine (2×10 mL), dried over anhydrous Na₂SO₄, filtered and concentrated under reduced pressure. The residue was purified by reverse-phase preparative HPLC. LCMS: m/z=419.0, 421.0 [M+H]⁺. ¹H NMR (400 MHz, CDCl₃): δ 8.95 (br s, 1H), 8.55 (s, 2H), 8.16 (d, J=8.0 Hz, 1H), 7.01 (d, J=8.0 Hz, 1H), 6.73 (d, J=1.2 Hz, 1H), 4.90 (br d, J=16.8 Hz, 1H), 4.77-4.51 (m, 1H), 4.40 (d, J=16.4 Hz, 1H), 4.17 (dd, J=1.6, 12.8 Hz, 1H), 3.55 (d, J=12.8 Hz, 1H), 1.72-1.64 (m, 1H), 1.58-1.50 (m, 2H), 1.45-1.34 (m, 1H), 1.17-1.08 (m, 2H).

Example 250 has been identified as a single enantiomer, N-(5-chloropyrimidin-2-yl)-2-[(2′R,4S)-2′-fluoro-6-(1-fluorocyclopropyl)-1-oxospiro[3H-isoquinoline-4,1′-cyclopropane]-2-yl]acetamide, as shown in Table 1A.

### Example 251 and 252

### 2-[6-[(1r,2r)-1,2-difluorocyclopropyl]-1-oxospiro[3H-isoquinoline-4,1′-cyclopropane]-2-yl]-N-(5-fluoropyrimidin-2-yl)acetamide (251 and 252)

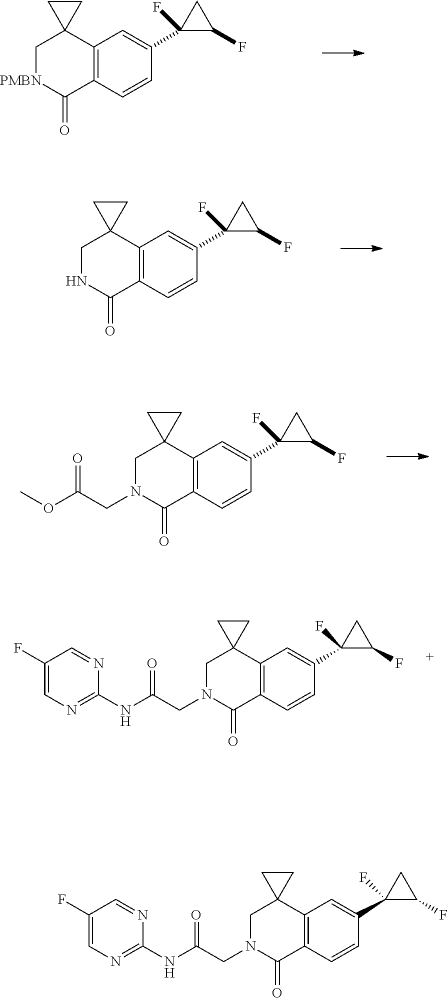

6′-((1r,2r)-1,2-difluorocyclopropyl)-2′,3′-dihydro-1′H-spiro[cyclopropane-1,4′-isoquinolin]-1′-one: 6-[(1r,2r)-1,2-difluorocyclopropyl]-2-[(4-methoxyphenyl)methyl]spiro[3H-isoquinoline-4,1′-cyclopropane]-1-one (350 mg, 0.95 mmol, Int. 54) was added to TFA (5.0 mL). The mixture was stirred at 60° C. for 10 h. The reaction mixture was quenched by addition of aq. sat. NaHCO₃ (20 mL) and extracted with EtOAc (3×10 mL). The combined organic layers were washed with brine (3×10 mL), dried over anhydrous Na₂SO₄, filtered, and concentrated under reduced pressure. The residue was purified by silica gel column chromatography. LCMS: m/z=250.1 [M+H]⁺.

methyl 2-(6′-((1r,2r)-1,2-difluorocyclopropyl)-1′-oxo-1′H-spiro[cyclopropane-1,4′-isoquinolin]-2′(3′H)-yl)acetate: To a solution of 6′-((1r,2r)-1,2-difluorocyclopropyl)-2′,3′-dihydro-1′H-spiro[cyclopropane-1,4′-isoquinolin]-1′-one (50 mg, 0.20 mmol) in DMF (2.0 mL) at 0° C. was added NaH (12 mg, 0.30 mmol, 60% purity). The mixture was stirred for 20 min at 0° C., then methyl 2-bromoacetate (61 mg, 0.40 mmol) was added. The mixture was stirred at 25° C. for 2 h. The reaction mixture was quenched by addition of aq. sat. NH₄Cl (10 mL) and extracted with EtOAc (3×10 mL). The combined organic layers were washed with brine (3×10 mL), dried over anhydrous Na₂SO₄, filtered, and concentrated under reduced pressure. The residue was purified by preparative silica gel thin-layer chromatography. LCMS: m/z=322.1 [M+H]⁺.

2-[6-[(1r,2r)-1,2-difluorocyclopropyl]-1-oxospiro[3H-isoquinoline-4,1′-cyclopropane]-2-yl]-N-(5-fluoropyrimidin-2-yl)acetamide: To a mixture of 5-fluoropyrimidin-2-amine (52 mg, 0.47 mmol) and methyl 2-(6′-((1r,2r)-1,2-difluorocyclopropyl)-1′-oxo-1′H-spiro[cyclopropane-1,4′-isoquinolin]-2′(3′H)-yl)acetate (50 mg, 0.16 mmol) in DCE (3.0 mL) was added AlMe₃ (1 M in heptane, 0.47 mL). The mixture was stirred at 80° C. for 2 h. The reaction mixture was quenched by addition of H₂O (10 mL) and extracted with DCM (3×10 mL). The combined organic layers were washed with brine (3×10 mL), dried over anhydrous Na₂SO₄, filtered, and concentrated under reduced pressure. The residue was purified by preparative silica gel thin-layer chromatography and further purified by chiral SFC (Column: Chiralpak IC-3 (50 mm×4.6 mm, 3 μm particle size); Mobile phase: A: CO₂ and B: 0.1% i-PrNH₂ in i-PrOH; Gradient: 50% B isocratic; Flow rate: 4 mL/min; Detection wavelength: 220 nm; Column temperature: 35° C.; System back pressure: 124 bar) to provide:

2-[6-[(1r,2r)-1,2-difluorocyclopropyl]-1-oxospiro[3H-isoquinoline-4,1′-cyclopropane]-2-yl]-N-(5-fluoropyrimidin-2-yl)acetamide (first eluting isomer, 251): LCMS: m/z=403.0 [M+H]⁺. ¹H NMR (400 MHz, CDCl₃) δ 9.07 (br s, 1H), 8.48 (s, 2H), 8.15 (dd, J=0.8, 8.0 Hz, 1H), 6.94 (d, J=8.2 Hz, 1H), 6.87 (d, J=1.6 Hz, 1H), 4.69-4.46 (m, 3H), 3.55 (s, 2H), 2.02-1.81 (m, 1H), 1.69-1.61 (m, 1H), 1.20-1.14 (m, 2H), 1.11-1.05 (m, 2H).

2-[6-[(1r,2r)-1,2-difluorocyclopropyl]-1-oxospiro[3H-isoquinoline-4,1′-cyclopropane]-2-yl]-N-(5-fluoropyrimidin-2-yl)acetamide (second eluting isomer, 252): LCMS: m/z=403.0 [M+H]⁺. ¹H NMR (400 MHz, CDCl₃) δ 9.04 (br s, 1H), 8.48 (s, 2H), 8.16 (dd, J=0.8, 8.0 Hz, 1H), 6.94 (d, J=8.2 Hz, 1H), 6.87 (d, J=1.6 Hz, 1H), 4.70-4.45 (m, 3H), 3.55 (s, 2H), 2.02-1.81 (m, 1H), 1.68-1.61 (m, 1H), 1.19-1.15 (m, 2H), 1.11-1.06 (m, 2H).

### Example 253 and 254

### 2-[6-[(1r,2s)-1,2-difluorocyclopropyl]-1-oxospiro[3H-isoquinoline-4,1′-cyclopropane]-2-yl]-N-(5-fluoropyrimidin-2-yl)acetamide (253 and 254)

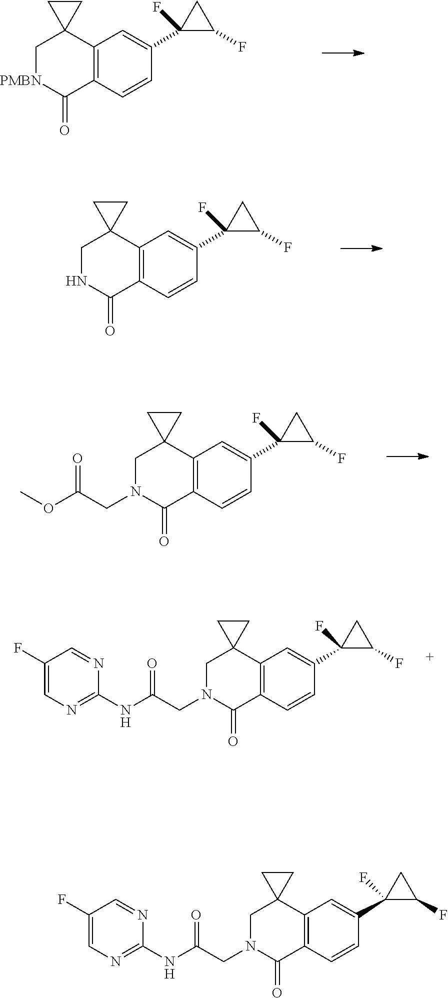

6′-((1r,2s)-1,2-difluorocyclopropyl)-2′,3′-dihydro-1′H-spiro[cyclopropane-1,4′-isoquinolin]-1′-one: 6-[(1r,2s)-1,2-difluorocyclopropyl]-2-[(4-methoxyphenyl)methyl]spiro[3H-isoquinoline-4,1′-cyclopropane]-1-one (350 mg, 0.95 mmol, Int. 53) was added to TFA (5.0 mL). The mixture was stirred at 60° C. for 10 h. The reaction mixture was quenched by addition of aq. sat. NaHCO₃ (20 mL) and extracted with EtOAc (3×10 mL). The combined organic layers were washed with brine (3×10 mL), dried over Na₂SO₄, filtered, and concentrated under reduced pressure. The residue was purified by silica gel column chromatography. LCMS: m/z=250.1 [M+H]⁺.

methyl 2-(6′-((1r,2s)-1,2-difluorocyclopropyl)-1′-oxo-1′H-spiro[cyclopropane-1,4′-isoquinolin]-2′(3′H)-yl)acetate: To a solution of 6′-((1r,2s)-1,2-difluorocyclopropyl)-2′,3′-dihydro-1′H-spiro[cyclopropane-1,4′-isoquinolin]-1′-one (80 mg, 0.32 mmol) in DMF (2 mL) at 0° C. was added NaH (19 mg, 0.48 mmol, 60% purity), the mixture was stirred at 0° C. for 15 min, then methyl 2-bromoacetate (98 mg, 0.64 mmol) was added at 0° C. The mixture was stirred at 25° C. for 2 h. The reaction mixture was quenched by addition of aq. sat. NH₄Cl (10 mL) and extracted with EtOAc (3×10 mL). The combined organic layers were washed with brine (3×10 mL), dried over anhydrous Na₂SO₄, filtered, and concentrated under reduced pressure. The residue was purified by preparative silica gel thin-layer chromatography. LCMS: m/z=322.1 [M+H]⁺.

2-[6-[(1r,2s)-1,2-difluorocyclopropyl]-1-oxospiro[3H-isoquinoline-4,1′-cyclopropane]-2-yl]-N-(5-fluoropyrimidin-2-yl)acetamide: To a mixture of 5-fluoropyrimidin-2-amine (63 mg, 0.56 mmol) and methyl 2-(6′-((1r,2s)-1,2-difluorocyclopropyl)-1′-oxo-1′H-spiro[cyclopropane-1,4′-isoquinolin]-2′(3′H)-yl)acetate (60 mg, 0.17 mmol) in DCE (3.0 mL) was added AlMe₃ (1 M heptane, 0.56 mL). The mixture was stirred at 80° C. for 2 h. The reaction mixture was quenched by addition of H₂O (10 mL) and extracted with DCM (3×10 mL). The combined organic layers were washed with brine (3×3 mL), dried over anhydrous Na₂SO₄, filtered, and concentrated under reduced pressure. The residue was purified by reverse-phase preparative HPLC and further purified by chiral SFC (Column: ChiralPak IF (50 mm×4.6 mm, 3.5 μm particle size); Mobile phase: A: CO₂ and B: 0.1% MeOH in i-PrOH; Gradient: 40% B isocratic; Flow rate: 4 mL/min; Detection wavelength: 220 nm; Column temperature: 35° C.; System back pressure: 100 bar) to provide:

2-[6-[(1r,2s)-1,2-difluorocyclopropyl]-1-oxospiro[3H-isoquinoline-4,1′-cyclopropane]-2-yl]-N-(5-fluoropyrimidin-2-yl)acetamide (first eluting isomer, 253): LCMS: m/z=403.0 [M+H]⁺. ¹H NMR (400 MHz, CDCl₃): δ 9.07 (br s, 1H), 8.48 (s, 2H), 8.20 (d, J=8.0 Hz, 1H), 7.33 (d, J=8.0 Hz, 1H), 6.96 (s, 1H), 5.16-4.94 (m, 1H), 4.56 (br s, 2H), 3.56 (s, 2H). 1.84-1.74 (m, 2H), 1.18-1.16 (m, 2H), 1.08-1.05 (m, 2H).

2-[6-[(1r,2s)-1,2-difluorocyclopropyl]-1-oxospiro[3H-isoquinoline-4,1′-cyclopropane]-2-yl]-N-(5-fluoropyrimidin-2-yl)acetamide (second eluting isomer, 254): LCMS: m/z=403.0 [M+H]⁺. ¹H NMR (400 MHz, CDCl₃): δ 9.11 (br s, 1H), 8.48 (s, 2H), 8.20 (d, J=8.0 Hz, 1H), 7.33 (d, J=8.0 Hz, 1H), 6.96 (s, 1H), 4.91-5.21 (m, 1H), 4.56 (br s, 2H), 3.56 (s, 2H). 1.91-1.69 (m, 2H), 1.19-1.16 (m, 2H), 1.11-1.02 (m, 2H).

### Example 255

### 2-[6-(1-fluorocyclopropyl)-1-oxospiro[3H-isoquinoline-4,1′-cyclopropane]-2-yl]-N-(5-fluoropyrimidin-2-yl)acetamide (255)

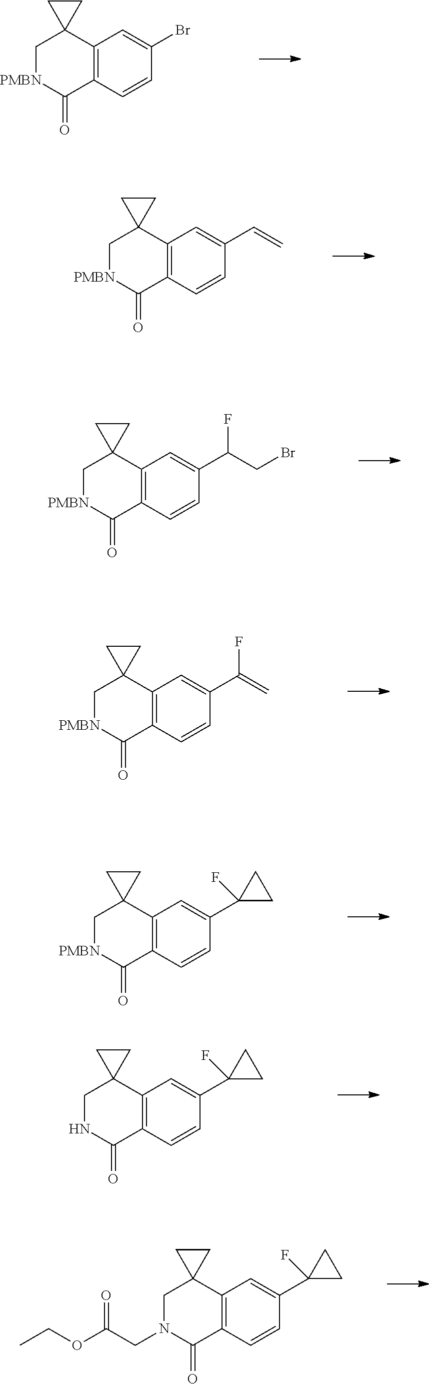

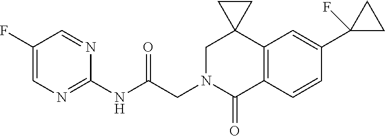 [SMILES: O=C(CN1CC2(CC2)c2cc(C3(F)CC3)ccc2C1=O)Nc1ncc(F)cn1]

2′-(4-methoxybenzyl)-6′-vinyl-2′,3′-dihydro-1′H-spiro[cyclopropane-1,4′-isoquinolin]-1′-one: To a mixture of 6′-bromo-2′-(4-methoxybenzyl)-2′,3′-dihydro-1′H-spiro[cyclopropane-1,4′-isoquinolin]-1′-one (5.5 g, 14.8 mmol, Int. 52) and potassium trifluoro(vinyl)borate (9.9 g, 73.9 mmol) in 1,4-dioxane (60 mL) were added CsF (6.73 g, 44.3 mmol) and Pd(dppf)Cl₂ (1.08 g, 1.48 mmol). The mixture was stirred at 90° C. for 12 h. The reaction mixture was quenched by addition of H₂O (100 mL) and extracted with EtOAc (3×30 mL). The combined organic layers were washed with brine (2×20 mL), dried over anhydrous Na₂SO₄, filtered, and concentrated under reduced pressure. The residue was purified by silica gel column chromatography. LCMS: m/z=320.1 [M+H]⁺.

6′-(2-bromo-1-fluoroethyl)-2′-(4-methoxybenzyl)-2′,3′-dihydro-1′H-spiro[cyclopropane-1,4′-isoquinolin]-1′-one: To a mixture of 2′-(4-methoxybenzyl)-6′-vinyl-2′,3′-dihydro-1′H-spiro[cyclopropane-1,4′-isoquinolin]-1′-one (4.0 g, 12.5 mmol) in DCM (60 mL) at 0° C. were added N,N-diethylethanamine;trihydrofluoride (6.06 g, 37.6 mmol) and NBS (2.67 g, 15.0 mmol). The mixture was stirred at 25° C. stirred for 16 h. The reaction mixture was quenched by addition of H₂O (100 mL) and extracted with DCM (3×30 mL). The combined organic layers were washed with brine (3×50 mL), dried over anhydrous Na₂SO₄, filtered, and concentrated under reduced pressure. The residue was purified by silica gel column chromatography. LCMS: m/z=417.9, 419.9 [M+H]⁺.

6′-(1-fluorovinyl)-2′-(4-methoxybenzyl)-2′,3′-dihydro-1′H-spiro[cyclopropane-1,4′-isoquinolin]-1′-one: To a mixture of 6′-(2-bromo-1-fluoroethyl)-2′-(4-methoxybenzyl)-2′,3′-dihydro-1′H-spiro[cyclopropane-1,4′-isoquinolin]-1′-one (2.4 g, 5.74 mmol) in DCM (25 mL) was added DBU (1.31 g, 8.61 mmol). The mixture was stirred at 50° C. for 12 h. The reaction mixture was quenched by addition of H₂O (20 mL) and extracted with DCM (3×30 mL). The combined organic layers were washed with brine (2×10 mL), dried over anhydrous Na₂SO₄, filtered, and concentrated under reduced pressure. The residue was purified by silica gel column chromatography. LCMS: m/z=338.1 [M+H]⁺.

6′-(1-fluorocyclopropyl)-2′-(4-methoxybenzyl)-2′,3′-dihydro-1′H-spiro[cyclopropane-1,4′-isoquinolin]-1′-one: To a solution of ZnEt₂ (1 M in toluene, 6.67 mL) at 0° C. was added dropwise CH₂I₂ (3.57 g, 13.3 mmol). The mixture was stirred at 0° C. for 1 h. 6′-(1-fluorovinyl)-2′-(4-methoxybenzyl)-2′,3′-dihydro-1′H-spiro[cyclopropane-1,4′-isoquinolin]-1′-one (450 mg, 1.33 mmol) in DCM (5 mL) was then added. The mixture was stirred at 20° C. for 15 h. The reaction mixture was quenched by addition of aq. sat. NH₄Cl (20 mL) and extracted with EtOAc (3×10 mL). The combined organic layers were washed with brine (20 mL), dried over anhydrous Na₂SO₄, filtered, and concentrated under reduced pressure. The residue was purified by silica gel column chromatography. LCMS: m/z=352.1 [M+H]⁺.

6′-(1-fluorocyclopropyl)-2′,3′-dihydro-1′H-spiro[cyclopropane-1,4′-isoquinolin]-1′-one: 6′-(1-fluorocyclopropyl)-2′-(4-methoxybenzyl)-2′,3′-dihydro-1′H-spiro[cyclopropane-1,4′-isoquinolin]-1′-one (230 mg, 0.65 mmol) was added to TFA (5 mL). The mixture was stirred at 60° C. for 16 h. The reaction mixture was quenched by addition of H₂O (10 mL) and extracted with DCM (3×10 mL). The combined organic layers were washed with brine (10 mL), dried over anhydrous Na₂SO₄, filtered, and concentrated under reduced pressure. The residue was purified by silica gel column chromatography. LCMS: m/z=232.1 [M+H]⁺.

ethyl 2-(6′-(1-fluorocyclopropyl)-1′-oxo-1′H-spiro[cyclopropane-1,4′-isoquinolin]-2′(3′H)-yl)acetate: To a mixture of 6-(1-fluorocyclopropyl)spiro[2,3-dihydroisoquinoline-4,1′-cyclopropane]-1-one (93 mg, 0.40 mmol) and ethyl 2-iodoacetate (172 mg, 0.80 mmol) in DMF (2.0 mL) was added Cs₂CO₃ (197 mg, 0.60 mmol). The mixture was stirred at 100° C. for 16 h and then 120° C. for another 5 h. The reaction mixture was cooled to 20° C., and then ethyl 2-iodoacetate (172 mg, 0.80 mmol) was added. The reaction was heated to 120° C., and stirred for another 8 h. The reaction mixture was then cooled to ambient temperature, diluted with H₂O (10 mL), and extracted with EtOAc (3×10 mL). The combined organic layers were washed with brine (10 mL), dried over anhydrous Na₂SO₄, filtered, concentrated under reduced pressure, and purified by silica gel column chromatography. LCMS: m/z=318.1 [M+H]⁺.

2-[6-(1-fluorocyclopropyl)-1-oxospiro[3H-isoquinoline-4,1′-cyclopropane]-2-yl]-N-(5-fluoropyrimidin-2-yl)acetamide: To a solution of ethyl 2-(6′-(1-fluorocyclopropyl)-1′-oxo-1′H-spiro[cyclopropane-1,4′-isoquinolin]-2′(3′H)-yl)acetate (60 mg, 0.19 mmol) and 5-fluoropyrimidin-2-amine (47 mg, 0.42 mmol) in DCE (1.0 mL) was added trimethyl-(4-trimethylalumanuidyl-1,4-diazoniabicyclo[2.2.2]octan-1-yl)alumanuide (63 mg, 0.25 mmol). The mixture was stirred at 60° C. for 12 h. The reaction mixture was filtered and the filtrate was concentrated under pressure. The residue was purified by reverse-phase preparative HPLC. LCMS: m/z=385.1 [M+H]⁺. ¹H NMR (400 MHz, CDCl₃): δ 9.09 (br s, 1H), 8.48 (s, 2H), 8.14 (dd, J=0.8, 8.0 Hz, 1H), 7.05-6.97 (m, 1H), 6.86 (d, J=1.6 Hz, 1H), 4.55 (br s, 2H), 3.55 (s, 2H), 1.58-1.53 (m, 2H), 1.19-1.03 (m, 6H).

### Example 256

### 2-(6-bromo-1-oxospiro[3H-isoquinoline-4,1′-cyclopropane]-2-yl)-N-(5-cyclobutylpyrimidin-2-yl)acetamide (256)

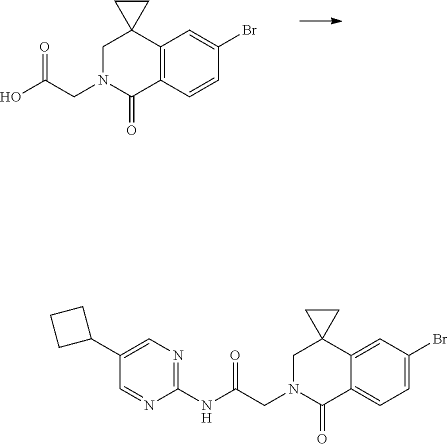 [SMILES: O=C(O)CN1CC2(CC2)c2cc(Br)ccc2C1=O ; O=C(CN1CC2(CC2)c2cc(Br)ccc2C1=O)Nc1ncc(C2CCC2)cn1]

To a solution of 5-cyclobutylpyrimidin-2-amine hydrochloride (67 mg, 0.45 mmol, Int. 55) and 2-(6-bromo-1-oxo-spiro[3H-isoquinoline-4,1′-cyclopropane]-2-yl)acetic acid (70 mg, 0.23 mmol, Int. 3) in pyridine (1.0 mL) was added EDCI (86 mg, 0.45 mmol), and the reaction mixture was stirred for 16 h. The reaction mixture was poured into aq. sat. NH₄Cl (10 mL) and extracted with EtOAc (3×5 mL). The combined organic layers were washed with brine (5 mL), dried over anhydrous Na₂SO₄, filtered, and concentrated under reduced pressure. The residue was purified by reverse-phase preparative HPLC. LCMS: m/z=441.1, 443.1 [M+H]⁺. ¹H NMR (400 MHz, CDCl₃): δ 8.69 (br s, 1H), 8.46 (s, 2H), 8.01 (d, J=8.8 Hz, 1H), 7.45 (d, J=8.4 Hz, 1H), 7.05-6.97 (m, 1H), 4.65 (s, 2H), 3.56-3.50 (m, 3H), 2.41-2.39 (m, 2H), 2.17-2.08 (m, 3H), 1.96-1.94 (m, 1H), 1.15-1.06 (m, 4H).

### Example 257

### 2-(6-bromo-1-oxospiro[3H-isoquinoline-4,1′-cyclopropane]-2-yl)-N-[5-(1-methylpyrazol-4-yl)pyrimidin-2-yl]acetamide (257)

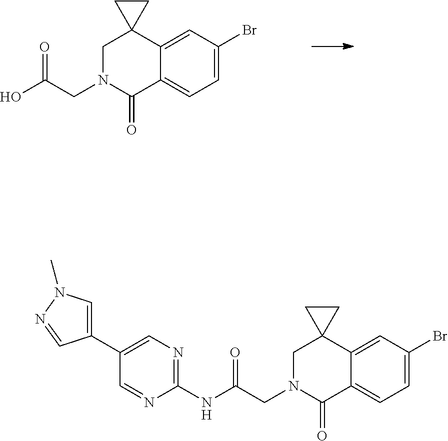 [SMILES: O=C(O)CN1CC2(CC2)c2cc(Br)ccc2C1=O ; Cn1cc(-c2cnc(NC(=O)CN3CC4(CC4)c4cc(Br)ccc4C3=O)nc2)cn1]

To a solution of 5-(1-methyl-1H-pyrazol-4-yl)pyrimidin-2-amine (54 mg, 0.31 mmol, Int. 56) and methyl 2-(6′-bromo-1′-oxo-1′H-spiro[cyclopropane-1,4′-isoquinolin]-2′(3′H)-yl)acetate (50 mg, 0.15 mmol, Int. 3) in toluene (2.0 mL) was added DABAL-Me₃ (39 mg, 0.15 mmol). The reaction mixture was stirred at 60° C. for 12 h. The reaction mixture was poured into H₂O (10 mL) and extracted with EtOAc (4×10 mL). The combined organic layers were washed with brine (10 mL), dried over anhydrous Na₂SO₄, filtered, and concentrated under reduced pressure. The residue was purified by reverse-phase preparative HPLC. LCMS: m/z=467.0, 469.0 [M+H]⁺. ¹H NMR (400 MHz, CDCl₃) δ 8.75 (br s, 1H), 8.68 (s, 2H), 8.03 (d, J=8.0 Hz, 1H), 7.75 (s, 1H), 7.66 (s, 1H), 7.48-7.45 (m, 1H), 7.01 (d, J=1.6 Hz, 1H), 4.66 (s, 2H), 3.99 (s, 3H), 3.55 (s, 2H), 1.16-1.13 (m, 2H), 1.10-1.07 (m, 2H).

### Example 258

### 2-(6-bromo-1-oxospiro[3H-isoquinoline-4,1′-cyclopropane]-2-yl)-N-[5-(oxolan-3-yl)pyrimidin-2-yl]acetamide (258)

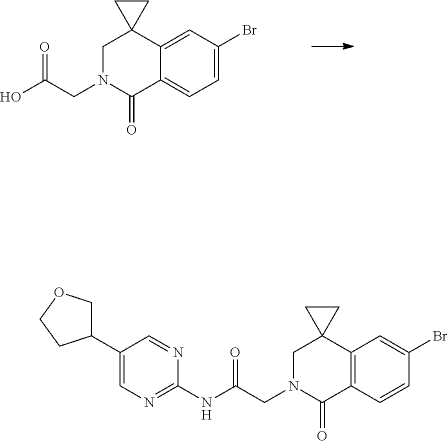 [SMILES: O=C(O)CN1CC2(CC2)c2cc(Br)ccc2C1=O ; O=C(CN1CC2(CC2)c2cc(Br)ccc2C1=O)Nc1ncc(C2CCOC2)cn1]

To a solution of 5-tetrahydrofuran-3-ylpyrimidin-2-amine (100 mg, 0.61 mmol, Int. 57), and 2-(6-bromo-1-oxo-spiro[3H-isoquinoline-4,1′-cyclopropane]-2-yl)acetic acid (225 mg, 0.73 mmol, Int. 3) in pyridine (1.5 mL) was added EDCI (348 mg, 1.82 mmol). The mixture was stirred at 15° C. for 16 h, and then stirred at 30° C. for 20 h. The reaction mixture was quenched by addition H₂O (3 mL) and extracted with DCM:MeOH (v:v=10:1, 5×2 mL). The combined organic layers were washed with brine (5 mL), dried over anhydrous Na₂SO₄, filtered, and concentrated under reduced pressure. The residue was purified by reverse-phase preparative HPLC. LCMS: m/z=456.9, 458.9 [M+H]⁺. ¹H NMR (400 MHz, DMSO-d₆): δ 10.78 (s, 1H), 8.59 (s, 2H), 7.80 (d, J=8.2 Hz, 1H), 7.52 (d, J=8.2 Hz, 1H), 7.26 (s, 1H), 4.53 (s, 2H), 4.01 (t, J=7.8 Hz, 1H), 3.98-3.92 (m, 1H), 3.80 (q, J=8.0 Hz, 1H), 3.58 (t, J=7.8 Hz, 1H), 3.48 (s. 2H), 3.40-3.37 (m, 1H), 2.37-2.27 (m, 1H), 1.98-1.93 (m, 1H), 1.18-1.13 (m, 2H), 1.08-1.01 (m, 2H)

### Example 259

### 2-(6-bromo-1-oxospiro[3H-isoquinoline-4,1′-cyclopropane]-2-yl)-N-(5-cyano-4-methylpyrimidin-2-yl)acetamide (259)

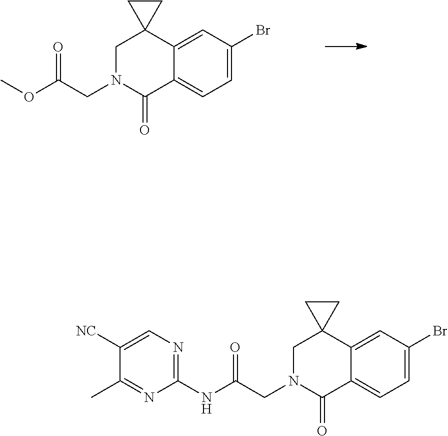 [SMILES: COC(=O)CN1CC2(CC2)c2cc(Br)ccc2C1=O ; Cc1nc(NC(=O)CN2CC3(CC3)c3cc(Br)ccc3C2=O)ncc1C#N]

2-(6-bromo-1-oxospiro[3H-isoquinoline-4,1′-cyclopropane]-2-yl)-N-(5-cyano-4-methylpyrimidin-2-yl)acetamide: To a solution of methyl 2-(6′-bromo-1′-oxo-1′H-spiro[cyclopropane-1,4′-isoquinolin]-2′(3′H)-yl)acetate (50 mg, 0.15 mmol, Int. 2) and 2-amino-4-methylpyrimidine-5-carbonitrile (41 mg, 0.31 mmol) in DCM (1.0 mL) at 0° C. was added AlMe₃ (1 M in n-heptane, 0.34 mL). The reaction mixture was stirred at 90° C. for 2 h. The reaction mixture was poured into H₂O (3 mL) and extracted with EtOAc (4×2 mL). The combined organic layers were washed with brine (5 mL), dried over anhydrous Na₂SO₄, filtered, and concentrated under reduced pressure. The residue was purified by preparative silica gel thin-layer chromatography. LCMS: m/z=425.9, 427.9 [M+H]⁺. ¹H NMR (400 MHz, CDCl₃) δ 8.87 (br s, 1H), 8.73 (s. 1H), 8.01 (d, J=8.3 Hz, 1H), 7.47 (d, J=8.8 Hz, 1H), 7.01 (s, 1H), 4.68 (s, 2H), 3.53 (s, 2H), 2.70 (s, 3H), 1.18-1.01 (m, 4H).

### Example 260

### 2-(6-bromo-1-oxospiro[3H-isoquinoline-4,1′-cyclopropane]-2-yl)-N-([1,2,4]triazolo[1,5-a]pyrazin-2-yl)acetamide (260)

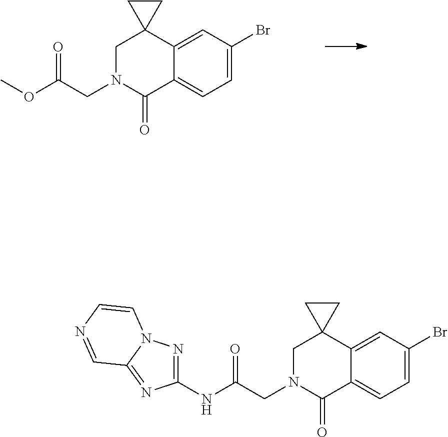 [SMILES: COC(=O)CN1CC2(CC2)c2cc(Br)ccc2C1=O ; O=C(CN1CC2(CC2)c2cc(Br)ccc2C1=O)Nc1nc2cnccn2n1]

To a solution of methyl 2-(6-bromo-1-oxo-spiro[3H-isoquinoline-4,1′-cyclopropane]-2-yl)acetate (25 mg, 0.08 mmol, Int. 2) and [1,2,4]triazolo[1,5-a]pyrazin-2-amine (26 mg, 0.19 mmol)

in DCE (0.5 mL) was added AlMe₃ (2 M in toluene, 0.12 mL). The reaction mixture was stirred for 1 h and then stirred at 90° C. for 30 min. The reaction mixture was cooled to 0° C., poured into H₂O (0.5 mL), filtered, and the filter cake was washed with EtOAc (5 mL). The filtrate was concentrated under reduced pressure and the residue was purified by reverse-phase preparative HPLC. LCMS: m/z=427.3, 429.3 [M+H]⁺. ¹H NMR (400 MHz, DMSO-d₆): δ 11.37 (s, 1H), 9.25 (d, J=1.4 Hz, 1H), 9.02 (dd, J=4.4, 1.4 Hz, 1H), 8.24 (d, J=4.4 Hz, 1H), 7.82 (d, J=8.3 Hz, 1H), 7.54 (dd, J=8.3, 1.9 Hz, 1H), 7.28 (d, J=1.9 Hz, 1H), 4.51 (s, 2H), 3.53 (s, 2H), 1.19-1.16 (m, 2H), 1.08-1.06 (m, 2H).

### Example 261

### 2-(6-bromo-1-oxospiro[3H-isoquinoline-4,1′-cyclopropane]-2-yl)-N-(7-chloro-[1,2,4]triazolo[1,5-a]pyridin-2-yl)acetamide (261)

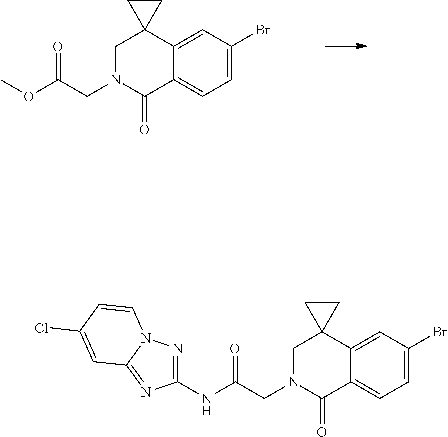 [SMILES: COC(=O)CN1CC2(CC2)c2cc(Br)ccc2C1=O ; O=C(CN1CC2(CC2)c2cc(Br)ccc2C1=O)Nc1nc2cc(Cl)ccn2n1]

To a solution of methyl 2-(6-bromo-1-oxo-spiro[3H-isoquinoline-4,1′-cyclopropane]-2-yl)acetate (25 mg, 0.08 mmol, Int. 2) and 7-chloro-[1,2,4]triazolo[1,5-a]pyridin-2-amine (32 mg, 0.19 mmol) in DCE (0.5 mL) was added AlMe₃ (2 M in toluene, 0.12 mL). The reaction mixture was stirred at 23° C. for 1 h and then at 90° C. for 30 min. The reaction mixture was cooled to 0° C., poured into H₂O (0.5 mL), filtered, and the filter cake was washed with EtOAc (5 mL). The filtrate was concentrated under reduced pressure and the residue was purified by reverse-phase preparative HPLC. LCMS: m/z=460.2, 462.2, 464.0 [M+H]⁺. ¹H NMR (400 MHz. DMSO-d₆): δ 9.50 (s, 1H), 8.49 (d, J=7.3 Hz, 1H), 8.04 (d, J=8.3 Hz, 1H), 7.64 (d, J=2.1 Hz, 1H), 7.48 (dd, J=8.3, 1.9 Hz, 1H), 7.02-6.99 (m, 2H), 4.51 (s, 2H), 3.57 (s, 2H), 1.17-1.13 (m, 2H), 1.12-1.08 (m, 2H).

### Example 262

### 2-(6-bromo-1-oxospiro[3H-isoquinoline-4,1′-cyclopropane]-2-yl)-N-(6-methoxy-[1,2,4]triazolo[1,5-a]pyridin-2-yl)acetamide (262)

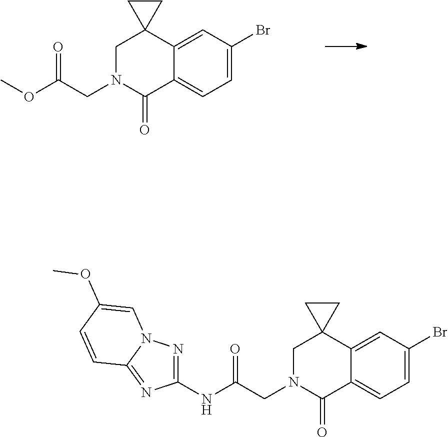 [SMILES: COC(=O)CN1CC2(CC2)c2cc(Br)ccc2C1=O ; COc1ccc2nc(NC(=O)CN3CC4(CC4)c4cc(Br)ccc4C3=O)nn2c1]

To a solution of methyl 2-(6-bromo-1-oxo-spiro[3H-isoquinoline-4,1′-cyclopropane]-2-yl)acetate (25 mg, 0.077 mmol, Int. 2) and 6-methoxy-[1,2,4]triazolo[1,5-a]pyridin-2-amine (32 mg, 0.19 mmol) in DCE (0.5 mL) was added AlMe₃ (2 M in toluene, 0.12 mL). The reaction mixture was stirred at 90° C. for 3 h. The reaction mixture was cooled to 0° C., poured into H₂O (0.5 mL), filtered, and the filter cake was washed with EtOAc (5 mL). The filtrate was concentrated under reduced pressure and the residue was purified by reverse-phase preparative HPLC. LCMS: m/z=456.3, 458.3 [M+H]⁺. H NMR (400 MHz, DMSO-d₆): δ 10.94 (s, 1H), 8.62 (dd, J=2.4, 0.7 Hz, 1H), 7.82 (d, J=8.3 Hz, 1H), 7.62 (dd, J=9.6, 0.6 Hz, 1H), 7.53 (dd, J=8.3, 1.9 Hz, 1H), 7.42 (dd, J=9.6, 2.4 Hz, 1H), 7.27 (d, J=1.9 Hz, 1H), 4.47 (br s, 2H), 3.85 (s, 3H), 3.52 (s, 2H), 1.18-1.16 (m, 2H), 1.08-1.05 (m, 2H).

### Example 263

### 2-(6-bromo-1-oxospiro[3H-isoquinoline-4,1′-cyclopropane]-2-yl)-N-(6-fluoro-[1,2,4]triazolo[1,5-a]pyridin-2-yl)acetamide (263)

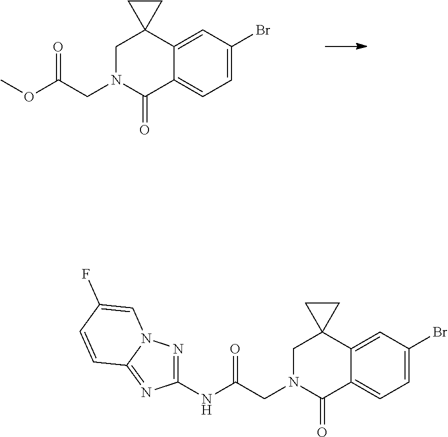 [SMILES: COC(=O)CN1CC2(CC2)c2cc(Br)ccc2C1=O ; O=C(CN1CC2(CC2)c2cc(Br)ccc2C1=O)Nc1nc2ccc(F)cn2n1]

To a solution of methyl 2-(6-bromo-1-oxo-spiro[3H-isoquinoline-4,1′-cyclopropane]-2-yl)acetate (25 mg, 0.08 mmol, Int. 2) and 6-fluoro-[1,2,4]triazolo[1,5-a]pyridin-2-amine (29 mg, 0.19 mmol) in DCE (0.5 mL) was added AlMe₃ (2 M in toluene, 0.12 mL). The reaction mixture was stirred at 23° C. for 1 h and then at 90° C. for 30 min The reaction mixture was cooled to 0° C., poured into H₂O (0.5 mL), filtered, and the filter cake was washed with EtOAc (5 mL). The filtrate was concentrated under reduced pressure and the residue was purified by reverse-phase preparative HPLC. LCMS: m/z=444.4, 446.4 [M+H]⁺. ¹H NMR (400 MHz, DMSO-d₆): δ 11.10 (s, 1H), 9.24 (ddd, J=4.1, 2.0, 1.3 Hz, 1H), 7.82 (d, J=8.3 Hz, 1H), 7.78-7.76 (m, 2H), 7.53 (dd, J=8.3, 1.9 Hz, 1H), 7.27 (d, J=1.9 Hz, 1H), 4.48 (s, 2H), 3.52 (s, 2H), 1.19-1.12 (m, 2H), 1.08-1.05 (m, 2H).

### Example 264

### 2-(6-bromo-1-oxospiro[3H-isoquinoline-4,1′-cyclopropane]-2-yl)-N-(6-chloro-[1,2,4]triazolo[1,5-a]pyridin-2-yl)acetamide (264)

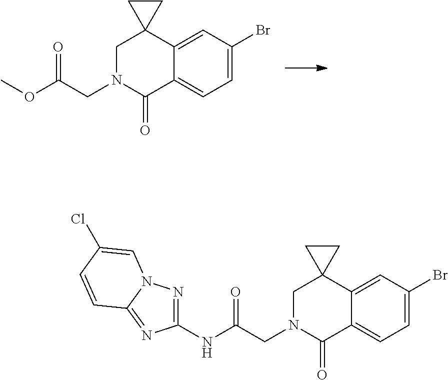 [SMILES: COC(=O)CN1CC2(CC2)c2cc(Br)ccc2C1=O ; O=C(CN1CC2(CC2)c2cc(Br)ccc2C1=O)Nc1nc2ccc(Cl)cn2n1]

To a solution of methyl 2-(6-bromo-1-oxo-spiro[3H-isoquinoline-4,1′-cyclopropane]-2-yl)acetate (25 mg, 0.077 mmol, Int. 2) and 6-chloro-[1,2,4]triazolo[1,5-a]pyridin-2-amine (33 mg, 0.19 mmol) in DCE (0.5 mL) was added AlMe₃ (2 M in toluene, 0.12 mL). The reaction mixture was stirred at 23° C. for 1 h and then at 90° C. for 30 min. The reaction mixture was cooled to 0° C., poured into H₂O (0.5 mL), filtered, and the filter cake was washed with EtOAc (5 mL). The filtrate was concentrated under reduced pressure and the residue was purified by reverse-phase preparative HPLC. LCMS: m/z=460.2, 462.2, 464.1 [M+H]⁺. ¹H NMR (400 MHz. DMSO-d₆): δ 11.14 (s, 1H), 9.25 (dd, J=1.8, 1.0 Hz, 1H), 7.82 (d, J=8.3 Hz, 1H), 7.74 (dd, J=2.7, 1.4 Hz, 2H), 7.53 (dd, J=8.3, 1.9 Hz, 1H), 7.27 (d, J=1.9 Hz, 1H), 4.49 (br s, 2H), 3.52 (s, 2H), 1.19-1.15 (m, 2H), 1.08-1.04 (m, 2H).

### Example 265

### 2-(6-bromo-1-oxospiro[3H-isoquinoline-4,1′-cyclopropane]-2-yl)-N-[7-(trifluoromethyl)-[1,2,4]triazolo[1,5-a]pyridin-2-yl]acetamide (265)

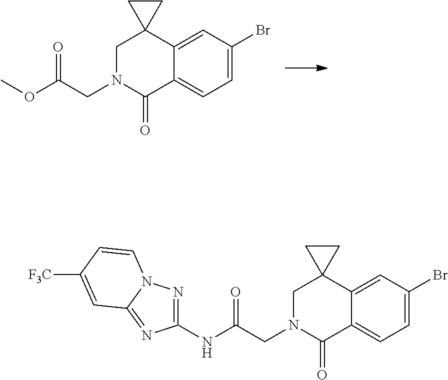 [SMILES: COC(=O)CN1CC2(CC2)c2cc(Br)ccc2C1=O ; O=C(CN1CC2(CC2)c2cc(Br)ccc2C1=O)Nc1nc2cc(C(F)(F)F)ccn2n1]

To a solution of methyl 2-(6-bromo-1-oxo-spiro[3H-isoquinoline-4,1′-cyclopropane]-2-yl)acetate (25 mg, 0.077 mmol, Int. 2) and 7-(trifluoromethyl)-[1,2,4]triazolo[1,5-a]pyridin-2-amine (39 mg, 0.19 mmol) in DCE (0.5 mL) was added AlMe₃ (2 M in toluene, 0.12 mL). The reaction mixture was stirred for 1 h and then stirred at 90° C. for 30 min. The reaction mixture was cooled to 0° C., poured into H₂O (0.5 mL), filtered, and the filter cake was washed with EtOAc (5 mL). The filtrate was concentrated under reduced pressure and the residue was purified by reverse-phase preparative HPLC. LCMS: m/z=494.3, 496.3 [M+H]⁺. ¹H NMR (400 MHz, DMSO-d₆): δ 11.30 (s, 1H), 9.11 (d, J=7.1 Hz, 1H), 8.27 (d, J=0.9 Hz, 1H), 7.82 (d, J=8.3 Hz, 1H), 7.54 (dd, J=8.3, 1.9 Hz, 1H), 7.44 (dd, J=7.1, 1.8 Hz, 1H), 7.28 (d, J=1.8 Hz, 1H), 4.51 (s, 2H), 3.53 (s, 2H), 1.19-1.12 (m, 2H), 1.09-1.05 (m, 2H).

### Example 267

### 2-(6-bromo-1-oxospiro[3H-isoquinoline-4,1′-cyclopropane]-2-yl)-N-(6-fluoro-1,3-benzoxazol-2-yl)acetamide (267)

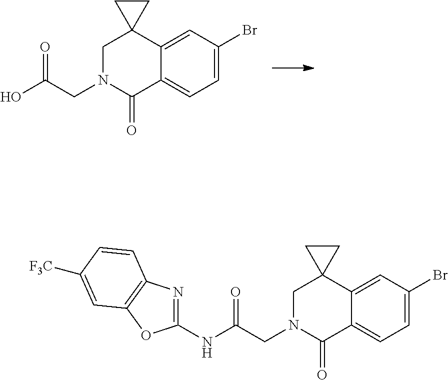 [SMILES: O=C(O)CN1CC2(CC2)c2cc(Br)ccc2C1=O ; O=C(CN1CC2(CC2)c2cc(Br)ccc2C1=O)Nc1nc2ccc(C(F)(F)F)cc2o1]

To a solution of 2-(6-bromo-1-oxo-spiro[3H-isoquinoline-4,1′-cyclopropane]-2-yl)acetic acid (42 mg, 0.14 mmol, Int. 3) in MeCN (1.3 mL) were added 6-fluoro-1,3-benzoxazol-2-amine (25 mg, 0.16 mmol), 1-methylimidazole (44 mg, 0.54 mmol), and chloro-N,N,N′,N′-tetramethylformamidinium hexafluorophosphate (48 mg, 0.17 mmol). The reaction mixture was stirred for 24 h. The reaction mixture was diluted with water (1 mL) and filtered. The filter cake was washed with water (2 ml) and MeCN (2 mL) and the residue was dried under reduced pressure. LCMS: m/z=444.3, 446.2 [M+H]⁺. ¹H-NMR (400 MHz, DMSO-d₆): δ 12.00 (s, 1H), 7.82 (d, J=8.3 Hz, 1H), 7.66 (dd, J=8.4, 2.5 Hz, 1H), 7.62-7.58 (m, 1H), 7.53 (dd, J=8.4, 1.8 Hz, 1H), 7.28-7.27 (m, 1H), 7.23-7.16 (m, 1H), 4.54 (s, 2H), 3.52 (s, 2H), 1.19-1.15 (m, 2H), 1.08-1.03 (m, 2H).

### Example 268

### 2-(6-bromo-1-oxospiro[3H-isoquinoline-4,1′-cyclopropane]-2-yl)-N-[(3R)-1-cyclopropylpiperidin-3-yl]acetamide (268)

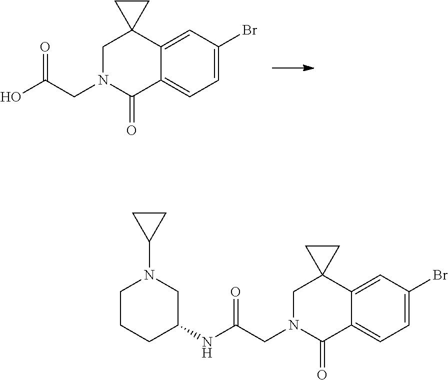 [SMILES: O=C(O)CN1CC2(CC2)c2cc(Br)ccc2C1=O ; O=C(CN1CC2(CC2)c2cc(Br)ccc2C1=O)N[C@@H]1CCCN(C2CC2)C1]

To a solution of 2-(6-bromo-1-oxo-spiro[3H-isoquinoline-4,1′-cyclopropane]-2-yl)acetic acid (30 mg, 0.1 mmol, Int. 3) and (3R)-1-cyclopropylpiperidin-3-amine (19 mg, 0.14 mmol) in DMF (1.0 mL) were added DIPEA (0.05 mL, 0.29 mmol) and T3P (0.07 mL, 0.13 mmol, 50% in EtOAc). The mixture was stirred for 16 h. The reaction mixture was diluted with 4 mL of a 4:1 mixture of MeCN and H₂O and directly purified by reverse-phase preparative HPLC. LCMS: m/z=432.5, 434.5 [M+H]⁺. H NMR (400 MHz, DMSO-d₆): δ 7.82-7.78 (m, 2H), 7.52 (dd, J=8.3, 1.9 Hz, 1H), 7.24 (d, J=1.9 Hz, 1H), 4.08 (s, 2H), 3.65-3.60 (m, 1H), 3.44-3.43 (m, 2H), 2.85-2.81 (m, 1H), 2.72-2.67 (m, 1H), 2.20-2.14 (m, 1H), 2.06-2.00 (m, 1H), 1.69-1.56 (m, 3H), 1.41-1.36 (m, 1H), 1.25-1.17 (m, 1H), 1.17-1.11 (m, 2H), 1.05-1.02 (m, 2H), 0.40-0.37 (m, 2H), 0.27-0.23 (m, 2H).

### Example 269

### 2-(6-bromo-1-oxospiro[3H-isoquinoline-4,1′-cyclopropane]-2-yl)-N-(5-iodopyrimidin-2-yl)acetamide (269)

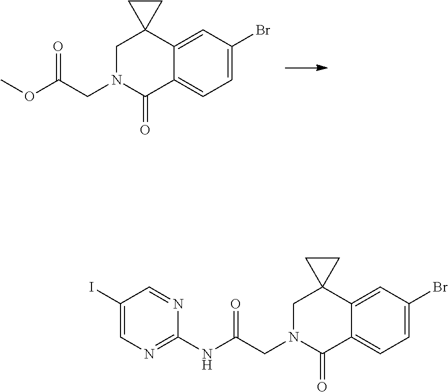 [SMILES: COC(=O)CN1CC2(CC2)c2cc(Br)ccc2C1=O ; O=C(CN1CC2(CC2)c2cc(Br)ccc2C1=O)Nc1ncc(I)cn1]

To a solution of 5-iodopyrimidin-2-amine (477 mg, 2.16 mmol) and methyl 2-(6′-bromo-1′-oxo-1′H-spiro[cyclopropane-1,4′-isoquinolin]-2′(3′H)-yl)acetate (350 mg, 1.08 mmol, Int. 2) in DCE (8 mL) was added AlMe₃ (1 M in n-heptane, 2.7 mL). The reaction mixture was stirred at 80° C. for 3 h The reaction mixture was poured into ice-cold H₂O (15 mL) and extracted with EtOAc (4×10 mL). The combined organic layers were washed with brine (2×10 mL), dried over anhydrous Na₂SO₄, filtered, and concentrated under reduced pressure. The residue was purified by reverse-phase preparative HPLC. LCMS: m/z=512.8, 514.9 [M+H]⁺. ¹H NMR (400 MHz, DMSO-d₆): δ 10.92 (s, 1H), 8.87 (s, 2H), 7.80 (d, J=8.4 Hz, 1H), 7.52 (dd, J=1.6, 8.4 Hz, 1H), 7.25 (d, J=1.6 Hz, 1H), 4.50 (s, 2H), 3.48 (s, 2H), 1.18-1.12 (m, 2H), 1.07-1.01 (m, 2H).

### Example 270

### 2-(6-bromo-1-oxospiro[3H-isoquinoline-4,1′-cyclopropane]-2-yl)-N-[5-(difluoromethoxy)pyrimidin-2-yl]acetamide (270)

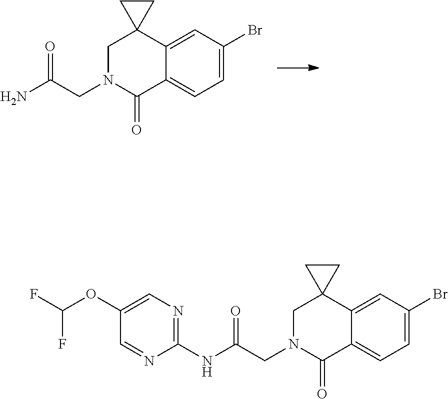 [SMILES: NC(=O)CN1CC2(CC2)c2cc(Br)ccc2C1=O ; O=C(CN1CC2(CC2)c2cc(Br)ccc2C1=O)Nc1ncc(OC(F)F)cn1]

To a solution of 2-chloro-5-(difluoromethoxy)pyrimidine (87 mg, 0.49 mmol) and 2-(6′-bromo-1′-oxo-1′H-spiro[cyclopropane-1,4′-isoquinolin]-2′(3′H)-yl)acetamide (100 mg, 0.32 mmol, Int. 58) in THF (1 mL) were added XPhos (15 mg, 0.03 mmol), Cs₂CO₃ (211 mg, 0.65 mmol), and Pd₂(dba)₃ (29 mg, 0.03 mmol). The reaction mixture was stirred at 50° C. for 12 h. The reaction mixture was diluted with H₂O (5 mL) and extracted with EtOAc (3×2 mL). The combined organic layers were washed with brine (3 mL), dried over anhydrous Na₂SO₄, filtered, and concentrated under reduced pressure. The residue was purified by reverse-phase preparative HPLC followed by preparative silica gel thin-layer chromatography. LCMS: m/z=452.9, 454.9 [M+H]⁺. ¹H NMR (400 MHz, CDCl₃): δ 9.08 (br s, 1H), 8.50 (br s, 2H), 8.02 (d, J=8.4 Hz, 1H), 7.46 (dd, J=2.0, 8.4 Hz, 1H), 7.00 (d, J=1.6 Hz, 1H), 6.55 (t, J=71.6 Hz, 1H), 4.62 (br s, 2H), 3.54 (s, 2H), 1.16-1.11 (m, 2H), 1.10-1.06 (m, 2H).

### Example 271

### 2-[(2′s,4r)-6-bromo-2′-fluoro-1-oxospiro[3H-isoquinoline-4,1′-cyclopropane]-2-yl]-N-([1,2,4]triazolo[1,5-a]pyridin-2-yl)acetamide (271)

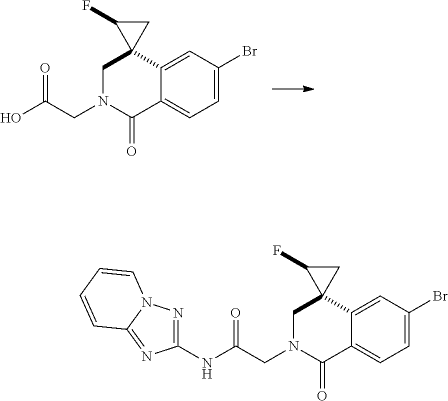

To a solution of 2-[(2′s,4r)-6-bromo-2′-fluoro-1-oxospiro[3H-isoquinoline-4,1′-cyclopropane]-2-yl]acetic acid (20 mg, 0.06 mmol, Int. 34) in pyridine (1.0 mL) were added [1,2,4]triazolo[1,5-a]pyridin-2-amine (20 mg, 0.15 mmol) and EDCI (23 mg, 0.12 mmol). The mixture was stirred for 3 h. The mixture was diluted with H₂O (4 mL) and extracted with EtOAc (3×1 mL). The combined organic layers were washed with brine (3 mL), dried over anhydrous Na₂SO₄, filtered, concentrated under reduced pressure, and purified by reverse-phase preparative HPLC. LCMS: m/z=444.0, 445.9 [M+H]⁺. ¹H NMR (400 MHz, CDCl₃): δ 9.47 (br s. 1H), 8.57 (br d, J=6.4 Hz, 1H), 8.06 (d, J=8.4 Hz, 1H), 7.66 (br d, J=8.4 Hz, 1H), 7.58-7.47 (m, 2H), 7.01 (br t, J=6.8 Hz, 1H), 6.85 (d, J=1.6 Hz, 1H), 4.92-4.26 (m, 3H), 4.19 (br d, J=12.8 Hz, 1H), 3.58 (br d, J=12.8 Hz, 1H), 1.65-1.61 (m, 1H), 1.49-1.38 (m, 1H).

Example 271 has been identified as a single enantiomer, 2-[(2′R,4S)-6-bromo-2′-fluoro-1-oxospiro[3H-isoquinoline-4,1′-cyclopropane]-2-yl]-N-([1,2,4]triazolo[1,5-a]pyridin-2-yl)acetamide, as shown in Table TA.

### Example 272

### 2-[(2′s,4r)-6-bromo-2′-fluoro-1-oxospiro[3H-isoquinoline-4,1′-cyclopropane]-2-yl]-N-(5-cyanopyrimidin-2-yl)acetamide (272)

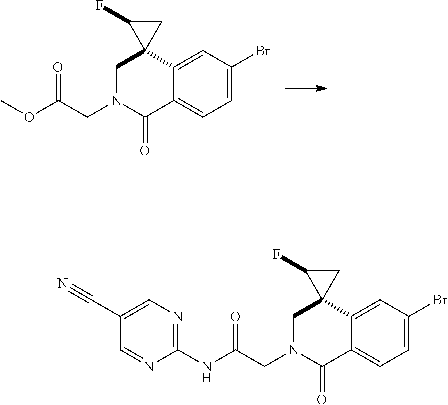

To a solution of methyl 2-[(2′s,4r)-6-bromo-2′-fluoro-1-oxospiro[3H-isoquinoline-4,1′-cyclopropane]-2-yl]acetate (70 mg, 0.20 mmol, Int. 33) in DCE (1 mL) were added 2-aminopyrimidine-5-carbonitrile (74 mg, 0.61 mmol) and AlMe₃ (1 M in heptane, 0.61 mL). The mixture was stirred at 60° C. for 5 h. The mixture was diluted with water (5 mL) and extracted with EtOAc (3×2 mL). The combined organic layers were washed with brine (3 mL), dried over anhydrous Na₂SO₄, filtered, concentrated under reduced pressure, and purified by reverse-phase preparative HPLC. LCMS: m/z=430.0, 431.9 [M+H]⁺. ¹H NMR (400 MHz, CDCl₃): δ 9.00 (br s, 1H), 8.85 (s, 2H), 8.05 (d, J=8.4 Hz, 1H), 7.54 (dd, J=1.6, 8.0 Hz, 1H), 6.86 (d, J=2.0 Hz, 1H), 4.97 (br d, 16.8 Hz, 1H), 4.72-4.50 (m, 1H), 4.43 (d, J=16.8 Hz, 1H), 4.23-4.15 (m, 1H), 3.52 (d, J=12.8 Hz, 1H), 1.70-1.61 (m, 1H), 1.47-1.36 (m, 1H).

Example 272 has been identified as a single enantiomer, 2-[(2′R,4S)-6-bromo-2′-fluoro-1-oxospiro[3H-isoquinoline-4,1′-cyclopropane]-2-yl]-N-(5-cyanopyrimidin-2-yl)acetamide, as shown in Table 1A.

### Example 273

### 2-[(2′s,4r)-2′-fluoro-6-(1-fluoroethenyl)-1-oxospiro[3H-isoquinoline-4,1′-cyclopropane]-2-yl]-N-(5-fluoropyrimidin-2-yl)acetamide (273)

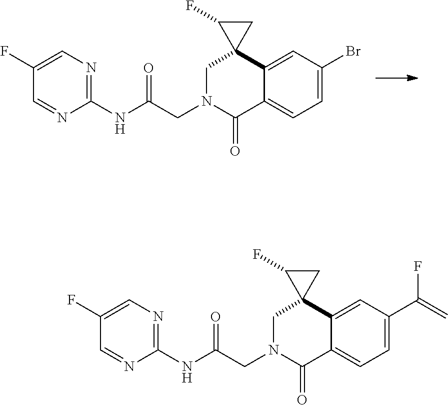

To a solution of 2-[(2′s,4r)-6-bromo-2′-fluoro-1-oxospiro[3H-isoquinoline-4,1′-cyclopropane]-2-yl]-N-(5-fluoropyrimidin-2-yl)acetamide (75 mg, 0.18 mmol, Ex. 79 in DMF (1.0 mL) were added (1-fluorovinyl)(methyl)diphenylsilane (107 mg, 0.44 mmol), CsF (67 mg, 0.44 mmol), CuI (3 mg, 0.02 mmol), and Pd(PPh₃)₄ (20 mg, 0.01 mmol). The mixture was stirred at 20° C. for 20 h. The mixture was diluted with H₂O (5 mL) and extracted with EtOAc (3×2 mL). The combined organic layers were washed with brine (3 mL), dried over anhydrous Na₂SO₄, filtered, concentrated under reduced pressure, and purified by reverse-phase preparative HPLC. LCMS: m/z=389.0 [M+H]⁺. H NMR (400 MHz, CDCl₃): δ 8.96 (br s, 1H), 8.49 (br s, 2H), 8.20 (br d, J=8.0 Hz, 1H), 7.56 (br d, J=8.0 Hz, 1H), 6.87 (s, 1H), 5.25-5.06 (m, 1H), 5.04-4.86 (m, 2H), 4.76-4.52 (m, 1H), 4.40 (br d, J=16.4 Hz, 1H), 4.19 (br d, J=12.8 Hz, 1H), 3.56 (br d, J=12.8 Hz, 1H), 1.71-1.64 (m, 1H), 1.47-1.36 (m, 1H).

Example 273 has been identified as a single enantiomer, 2-[(2′R,4S)-2′-fluoro-6-(1-fluoroethenyl)-1-oxospiro[3H-isoquinoline-4,1′-cyclopropane]-2-yl]-N-(5-fluoropyrimidin-2-yl)acetamide, as shown in Table 1A.

### Example 274

### 2-[(2′s,4r)-6-bromo-2′-fluoro-1-oxospiro[3H-isoquinoline-4,1′-cyclopropane]-2-yl]-N-(5-methylpyrimidin-2-yl)acetamide (274)

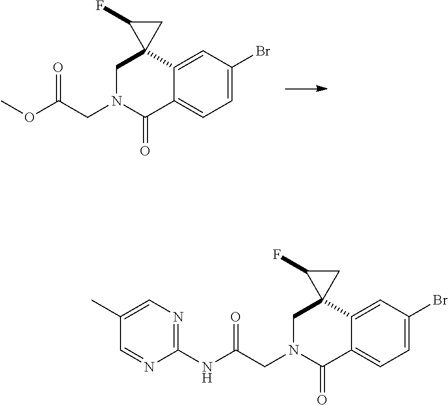

To a solution of methyl 2-[(2′s,4r)-6-bromo-2′-fluoro-1-oxospiro[3H-isoquinoline-4,1′-cyclopropane]-2-yl]acetate (50 mg, 0.14 mmol, Int. 33) in DCE (2.0 mL) were added 5-methylpyrimidin-2-amine (19 mg, 0.18 mmol) and AlMe₃ (1 M in heptane, 0.22 mL). The mixture was stirred at 60° C. for 16 h. The reaction was quenched with H₂O (6 mL) and extracted with EtOAc (3×6 mL). The combined organic layers were washed with brine (6 mL), dried over anhydrous Na₂SO₄, filtered, and concentrated under reduced pressure. The residue was purified by reverse-phase preparative HPLC. LCMS: m/z=418.0, 420.0 [M+H]⁺. ¹H NMR (400 MHz, CDCl₃) δ 8.91 (br s, 1H), 8.44 (s, 2H), 8.05 (d, J=8.4 Hz, 1H), 7.51 (dd, J=1.2, 8.4 Hz, 1H), 6.84 (s, 1H), 5.24-4.97 (m, 1H), 4.78-4.50 (m, 1H), 4.43 (br d, J=16.8 Hz, 1H), 4.18 (br d, J=12.8 Hz, 1H), 3.53 (d, J=12.8 Hz, 1H), 2.27 (s, 3H), 1.60-1.52 (m, 1H), 1.48-1.34 (m, 1H).

Example 274 has been identified as a single enantiomer, 2-[(2′R,4S)-6-bromo-2′-fluoro-1-oxospiro[3H-isoquinoline-4,1′-cyclopropane]-2-yl]-N-(5-methylpyrimidin-2-yl)acetamide, as shown in Table 1A.

### Example 275

### 2-[(2′r,4s)-6-bromo-2′-fluoro-1-oxospiro[3H-isoquinoline-4,1′-cyclopropane]-2-yl]-N-(5-cyano-3-fluoropyridin-2-yl)acetamide (275)

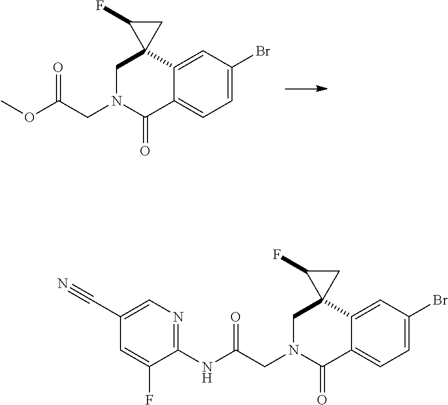

To a solution of 6-amino-5-fluoronicotinonitrile (48.1 mg, 0.35 mmol, Int. 59) and methyl 2-[(2′s,4r)-6-bromo-2′-fluoro-1-oxospiro[3H-isoquinoline-4,1′-cyclopropane]-2-yl]acetate (40 mg, 0.12 mmol, Int. 33) in DCE (1.0 mL) was added AlMe₃ (1 M in n-heptane, 0.18 mL). The reaction mixture was stirred at 80° C. for 3 h. The reaction mixture was quenched by addition aq. sat. NH₄Cl (3 mL) and extracted with EtOAc (3×1 mL). The combined organic layers were washed with brine (1 mL), dried over anhydrous Na₂SO₄, filtered, and concentrated under reduced pressure. The residue was purified by reverse-phase preparative HPLC. LCMS: m/z=446.9, 448.9 [M+H]⁺. ¹H NMR (400 MHz, CDCl₃): δ 8.90 (br s, 1H), 8.50 (d, J=1.6 Hz, 1H), 8.04 (d, J=8.4 Hz, 1H), 7.68 (dd, J=1.6, 9.6 Hz, 1H), 7.54 (dd, J=1.6, 8.4 Hz, 1H), 6.86 (d, J=1.6 Hz, 1H), 4.86 (br d, J=16.2 Hz, 1H), 4.72-4.49 (m, 1H), 4.39 (d, J=16.4 Hz, 1H), 4.19 (dd, J=1.6, 12.8 Hz, 1H), 3.56 (d, J=12.8 Hz, 1H), 1.66-1.62 (m, 1H), 1.49-1.37 (m, 1H).

Example 275 has been identified as a single enantiomer, 2-[(2′R,4S)-6-bromo-2′-fluoro-1-oxospiro[3H-isoquinoline-4,1′-cyclopropane]-2-yl]-N-(5-cyano-3-fluoropyridin-2-yl)acetamide, as shown in Table 1A.

### Example 276

### 2-[(2′s,4r)-6-chloro-2′-fluoro-1-oxospiro[3H-isoquinoline-4,1′-cyclopropane]-2-yl]-N-(5-fluoropyrimidin-2-yl)acetamide (276)

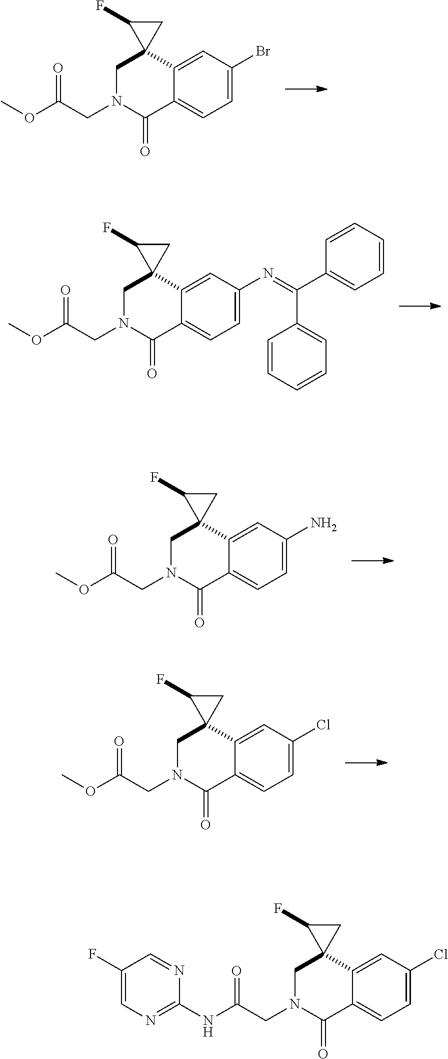

methyl 2-[(2′s,4r)-2′-fluoro-6-((diphenylmethylene)amino)-1-oxo-spiro[3H-isoquinoline-4,1′-cyclopropane]-2-yl]acetate: To a solution of methyl 2-[(2′s,4r)-6-bromo-2′-fluoro-1-oxospiro[3H-isoquinoline-4,1′-cyclopropane]-2-yl]acetate (500 mg, 1.46 mmol, Int. 33) and diphenylmethanimine (530 mg, 2.92 mmol) in 1,4-dioxane (5.0 mL) were added Cs₂CO₃ (952 mg, 2.92 mmol), XPhos (139 mg, 0.29 mmol), and Pd₂(dba)₃ (134 mg, 0.15 mmol). The reaction mixture was stirred at 90° C. for 16 h. The reaction mixture was filtered, concentrated under reduced pressure, and the residue was purified by silica gel column chromatography. LCMS: m/z=443.2 [M+H]⁺.

methyl 2-[(2′s,4r)-6-amino-2′-fluoro-1-oxo-spiro[3H-isoquinoline-4,1′-cyclopropane]-2-yl]acetate: To a solution of methyl 2-[(2′s,4r)-2′-fluoro-6-((diphenylmethylene)amino)-1-oxo-spiro[3H-isoquinoline-4,1′-cyclopropane]-2-yl]acetate (340 mg, 0.77 mmol) in MeOH (10 mL) were added NH₂OH·HCl (107 mg, 1.54 mmol) and NaOAc (189 mg, 2.31 mmol). The reaction mixture was stirred for 16 h. The reaction mixture was poured into aq. sat. NaHCO₃ (15 mL) and extracted with EtOAc (3×10 mL). The combined organic layers were washed with brine (10 mL), dried over anhydrous Na₂SO₄, filtered, and concentrated under reduced pressure to afford a residue that was purified by silica gel column chromatography. LCMS: m/z=279.1 [M+H]⁺.

methyl 2-[(2′s,4r)-6-chloro-2′-fluoro-1-oxo-spiro[3H-isoquinoline-4,1′-cyclopropane]-2-yl]acetate: To a solution of CuCl (35 mg, 0.36 mmol) in MeCN (4 mL) at 60° C. was added a solution of tert-butyl nitrite (33 mg, 0.32 mmol) and methyl 2-[(2′s,4r)-6-amino-2′-fluoro-1-oxo-spiro[3H-isoquinoline-4,1′-cyclopropane]-2-yl]acetate (50 mg, 0.18 mmol) in MeCN (1.0 mL). The reaction mixture was stirred at 60° C. for 5 h. The reaction mixture was poured into aq. sat. NaHCO₃ (10 mL) and extracted with EtOAc (3×10 mL). The organic layers were dried over anhydrous Na₂SO₄, filtered, and concentrated under reduced pressure. The residue was purified by silica gel column chromatography. LCMS: m/z=298.1, 300.1 [M+H]⁺.

2-[(2′s,4r)-6-chloro-2′-fluoro-1-oxospiro[3H-isoquinoline-4,1′-cyclopropane]-2-yl]-N-(5-fluoropyrimidin-2-yl)acetamide: To a solution of methyl 2-[(2′s,4r)-6-chloro-2′-fluoro-1-oxo-spiro[3H-isoquinoline-4,1′-cyclopropane]-2-yl]acetate (40 mg, 0.13 mmol) and 5-fluoropyrimidin-2-amine (22.8 mg, 0.2 mmol) in DCE (2.0 mL) was added AlMe₃ (1 M in heptane, 0.27 mL). The reaction mixture was stirred at 90° C. for 1.5 h. The reaction mixture was diluted with H₂O (5 mL) and extracted with EtOAc (3×5 mL). The organic layers were dried over anhydrous Na₂SO₄, filtered, and concentrated under reduced pressure. The residue was purified by reverse-phase preparative HPLC. LCMS: m/z=379.0, 380.9 [M+H]⁺. ¹H NMR (400 MHz, CDCl₃): δ 8.99 (s, 1H), 8.48 (s, 2H), 8.13 (d, J=8.4 Hz, 1H), 7.35 (dd, J=8.4, 2.0 Hz, 1H), 6.69 (d, J=2.0 Hz, 1H), 5.03-4.85 (m, 1H), 4.75-4.49 (m, 1H), 4.38 (d, J=16.4 Hz, 1H), 4.19 (dd, J=12.8, 2.0 Hz, 1H), 3.54 (d, J=12.8 Hz, 1H), 1.61-1.56 (m, 1H), 1.50-1.32 (m, 1H).

Example 276 has been identified as a single enantiomer, 2-[(2′R,4S)-6-chloro-2′-fluoro-1-oxospiro[3H-isoquinoline-4,1′-cyclopropane]-2-yl]-N-(5-fluoropyrimidin-2-yl)acetamide, as shown in Table 1A.

### Example 277 and 278

### 2-[(2′s,3′s,4r)-6-bromo-2′-fluoro-3′-methyl-1-oxospiro[3H-isoquinoline-4,1′-cyclopropane]-2-yl]-N-(5-fluoropyrimidin-2-yl)acetamide (277 and 278)

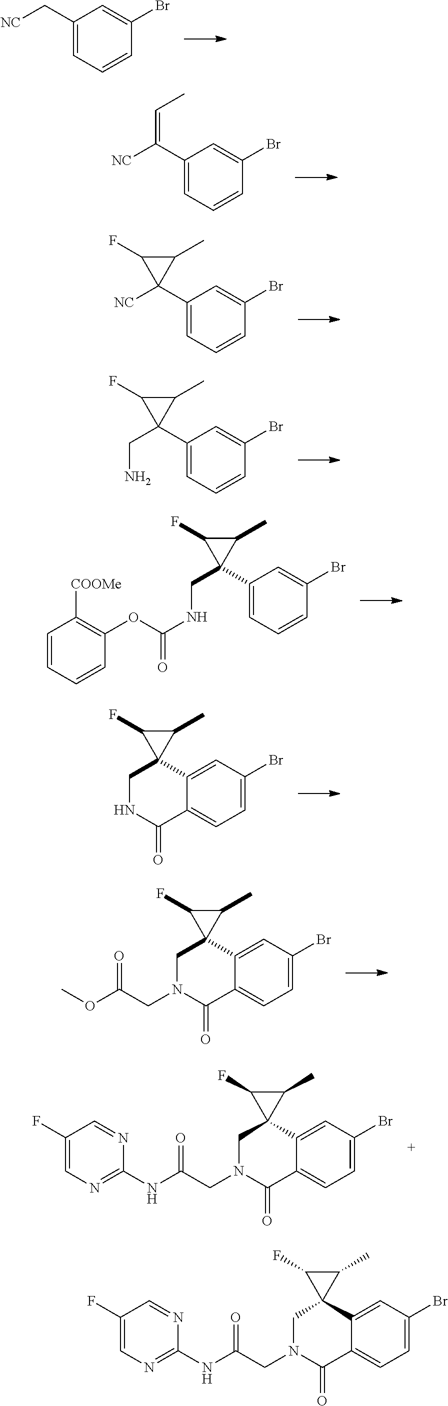

(E)-2-(3-bromophenyl)but-2-enenitrile: A mixture of acetaldehyde (16.9 g, 153 mmol), K₂CO₃ (14.0 g, 102 mmol), and 2-(3-bromophenyl)acetonitrile (10 g, 51 mmol) in MeOH (100 mL) was stirred for 4 h. The reaction mixture was poured into sat. aq. NH₄Cl (100 mL) and extracted with EtOAc (3×100 mL). The combined organic layers were washed with brine (50 mL), dried over anhydrous Na₂SO₄, filtered, concentrated under reduced pressure, and purified by silica gel column chromatography.

1-(3-bromophenyl)-2-fluoro-3-methylcyclopropanecarbonitrile: To a solution of (E)-2-(3-bromophenyl)but-2-enenitrile (5 g, 22.5 mmol) in THF (50 mL) at 0° C. were added (fluoromethyl)(phenyl)(2,3,4,5-tetramethylphenyl)sulfonium tetrafluoroborate (9.4 g, 24.8 mmol) and NaH (2.70 g, 67.5 mmol, 60% purity). The mixture was stirred at 15° C. for 16 h. The reaction mixture was poured into H₂O (300 mL) and extracted with EtOAc (3×300 mL). The combined organic layers were washed with brine (300 mL), dried over anhydrous Na₂SO₄, filtered, concentrated under reduced pressure, and purified by silica gel column chromatography.

(1-(3-bromophenyl)-2-fluoro-3-methylcyclopropyl)methanamine: To a solution of 1-(3-bromophenyl)-2-fluoro-3-methyl-cyclopropanecarbonitrile (3.3 g, 13 mmol) in THF (100 mL) at 0° C. was added BH₃ (1 M in THF, 39 mL). The solution was stirred for 12 h at 20° C. The reaction mixture was poured into MeOH (50 mL) and stirred for 1 h. The solution was concentrated under reduced pressure and the crude residue was dissolved in aq. HCl (1 M, 15 mL). The mixture was washed with MTBE (3×10 mL) and the pH of the aqueous layer was adjusted to pH=10 with NaOH (2N, 40 mL). The mixture was extracted with DCM (3×15 mL). The combined organic layers were washed with brine (10 mL), dried over anhydrous Na₂SO₄, filtered, and concentrated under reduced pressure to provide a residue that was used directly. LCMS: m/z=258.0, 260.0 [M+H]⁺.

methyl 2-(((((2s,3s)-1-(3-bromophenyl)-2-fluoro-3-methylcyclopropyl)methyl)carbamoyl)oxy)benzoate: To a solution of (1-(3-bromophenyl)-2-fluoro-3-methyl-cyclopropyl)methanamine (1.6 g, 6.20 mmol) in THF (20 mL) was added methyl 2-(2-methoxycarbonylphenoxy)carbonyloxybenzoate (3.07 g, 9.30 mmol). The mixture was stirred for 16 h. The mixture was concentrated under reduced pressure to provide a residue that was purified by reverse-phase preparative HPLC. LCMS: m/z=458.1, 460.1 [M+Na]⁺.

(1r,2s,3s)-6′-bromo-2-fluoro-3-methyl-2′,3′-dihydro-1′H-spiro[cyclopropane-1,4′-isoquinolin]-1′-one: To a solution of methyl 2-(((((2s,3s)-1-(3-bromophenyl)-2-fluoro-3-methylcyclopropyl)methyl)carbamoyl)oxy)benzoate (700 mg, 1.60 mmol) in DCM (10 mL) at 0° C. was added TfOH (2.41 g, 16.1 mmol). The mixture was stirred at 20° C. for 1 h. The reaction mixture was quenched with water (20 mL) and extracted with EtOAc (3×10 mL). The combined organic layers were washed with brine (10 mL), dried over anhydrous Na₂SO₄, filtered, concentrated under reduced pressure, and purified by reverse-phase preparative HPLC. LCMS: m/z=284.0, 286.0 [M+H]⁺.

methyl 2-((1r,2s,3s)-6′-bromo-2-fluoro-3-methyl-1′-oxo-1′H-spiro[cyclopropane-1,4′-isoquinolin]-2′(3′H)-yl)acetate: To a solution of (1r,2s,3s)-6′-bromo-2-fluoro-3-methyl-2′,3′-dihydro-1′H-spiro[cyclopropane-1,4′-isoquinolin]-1′-one (93 mg, 0.33 mmol) in DMF (1.0 mL) at 0° C. was added NaH (20 mg, 0.49 mmol, 60% purity). The mixture was stirred at 0° C. for 30 min. Methyl 2-bromoacetate (100 mg, 0.65 mmol) was added to the mixture at 0° C. The mixture was stirred at 20° C. for 1.5 h. The reaction mixture was poured into H₂O (10 mL) and extracted with EtOAc (3×5 mL). The combined organic layers were washed with brine (10 mL), dried over anhydrous Na₂SO₄, filtered, concentrated under reduced pressure, and purified by silica gel column chromatography. LCMS: m/z=356.2, 358.2 [M+H]⁺. ¹H NMR (400 MHz, CDCl₃): δ 8.00 (d, J=8.4 Hz, 1H), 7.47 (dd, J=1.6, 8.4 Hz, 1H), 6.85 (d, J=1.2 Hz, 1H), 4.77-4.56 (m, 1H), 4.53 (d, J=17.2 Hz, 1H), 4.14 (d, J=17.2 Hz, 1H), 3.82 (dd, J=2.4, 12.4 Hz, 1H), 3.77 (s, 3H), 3.58 (d, J=13.2 Hz, 1H), 1.68-1.57 (m, 1H), 1.29 (d, J=6.4 Hz, 3H).

2-[(2′s,3′s,4r)-6-bromo-2′-fluoro-3′-methyl-1-oxospiro[3H-isoquinoline-4,1′-cyclopropane]-2-yl]-N-(5-fluoropyrimidin-2-yl)acetamide: To a solution of 2-((1r,2s,3s)-6′-bromo-2-fluoro-3-methyl-1′-oxo-1′H-spiro[cyclopropane-1,4′-isoquinolin]-2′(3′H)-yl)acetate (160 mg, 0.45 mmol) and 5-fluoropyrimidin-2-amine (101 mg, 0.9 mmol) in DCE (2 mL) was added AlMe₃ (1 M in heptane, 0.9 mL). The mixture was stirred at 60° C. for 3 h. The reaction mixture was diluted with H₂O (1 mL) and extracted with EtOAc (3×10 mL). The combined organic layers were washed with brine (3 mL), dried over anhydrous Na₂SO₄, filtered, concentrated under reduced pressure, and purified by reverse-phase preparative HPLC. The mixture was further purified by chiral SFC (column: Chiralpak IC-3 (50 mm×4.6 mm, 3 μm particle size); Mobile phase: A: CO₂ B: i-PrOH (0.1%, i-PrOH); Gradient: B %=40% isocratic elution mode; Flow rate: 4 mL/min; Detection Wavelength: 220 nm; Column temperature 35° C.; System back pressure: 124 bar) to provide:

2-[(2′s,3′s,4r)-6-bromo-2′-fluoro-3′-methyl-1-oxospiro[3H-isoquinoline-4,1′-cyclopropane]-2-yl]-N-(5-fluoropyrimidin-2-yl)acetamide (first eluting isomer, 277): LCMS: m/z=437.1, 439.1 [M+H]⁺. H NMR (400 MHz, CDCl₃): δ 8.92 (br s, 1H), 8.48 (s, 2H), 8.04 (d, J=8.4 Hz, 1H), 7.50 (dd, J=2.0, 8.4 Hz, 1H), 6.88 (d, J=1.6 Hz, 1H), 4.81 (br d, J=15.6 Hz, 1H), 4.75-4.56 (m, 1H), 4.46 (br d, J=16.4 Hz, 1H), 3.92 (dd, J=2.4, 13.2 Hz, 1H), 3.67 (d, J=13.2 Hz, 1H), 1.71-1.63 (m, 1H), 1.30 (d, J=6.8 Hz, 3H).

2-[(2′s,3′s,4r)-6-bromo-2′-fluoro-3′-methyl-1-oxospiro[3H-isoquinoline-4,1′-cyclopropane]-2-yl]-N-(5-fluoropyrimidin-2-yl)acetamide (second eluting isomer, 278): LCMS: m/z=437.1, 439.1 [M+H]⁺. H NMR (400 MHz, CDCl₃): δ 8.89 (br s, 1H), 8.48 (s, 2H), 8.04 (d, J=8.4 Hz, 1H), 7.50 (dd, J=2.0, 8.4 Hz, 1H), 6.88 (d, J=1.6 Hz, 1H), 4.80 (br d, J=14.8 Hz, 1H), 4.76-4.52 (m, 1H), 4.46 (br d, J=16.4 Hz, 1H), 3.92 (dd, J=2.4, 13.2 Hz, 1H), 3.67 (d, J=13.2 Hz, 1H), 1.70-1.61 (m, 1H), 1.30 (d, J=6.8 Hz, 3H).

### Example 279, 280, 281, and 282

### 2-[(2′s,4s)-6-bromo-2′-methyl-1-oxospiro[3H-isoquinoline-4,1′-cyclopropane]-2-yl]-N-(5-fluoropyrimidin-2-yl)acetamide and 2-[(2′s,4r)-6-bromo-2′-methyl-1-oxospiro[3H-isoquinoline-4,1′-cyclopropane]-2-yl]-N-(5-fluoropyrimidin-2-yl)acetamide (279, 280, 281, and 282)

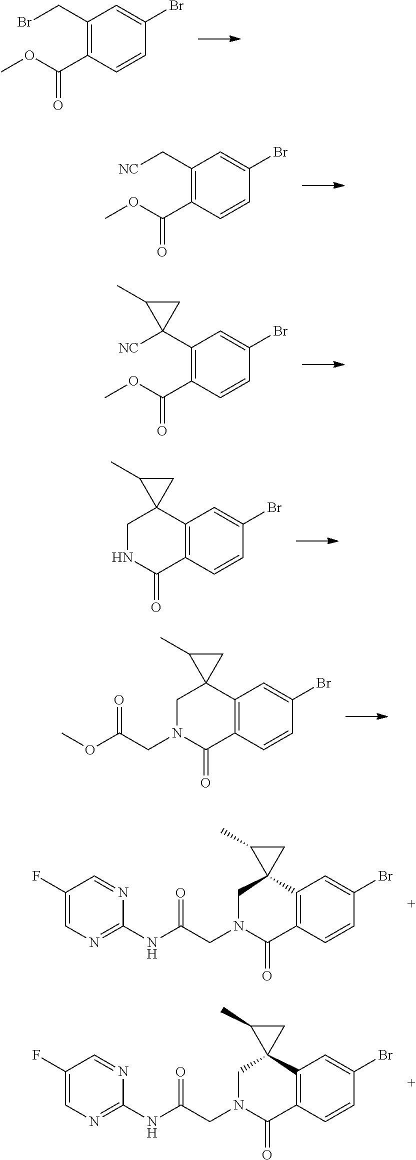

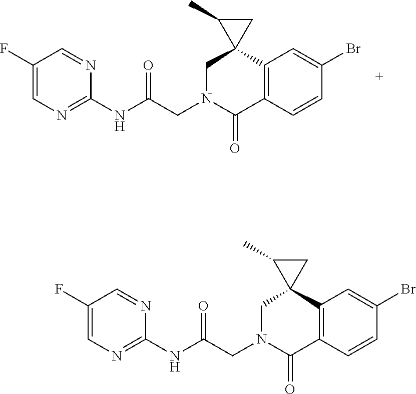 [SMILES: C[C@H]1C[C@@]12CN(CC(=O)Nc1ncc(F)cn1)C(=O)c1ccc(Br)cc12 ; C[C@@H]1C[C@]12CN(CC(=O)Nc1ncc(F)cn1)C(=O)c1ccc(Br)cc12]

methyl 4-bromo-2-(cyanomethyl)benzoate: To a mixture of methyl 4-bromo-2-(bromomethyl)benzoate (100 g, 324 mmol) in 1,4-dioxane (1000 mL) and H₂O (375 mL) at 0° C. was added NaCN (24.3 g, 495 mmol). The mixture was stirred at 25° C. for 10 h. The reaction mixture was poured into brine (1000 mL). The mixture was extracted with EtOAc (3×500 mL). The combined organic layers were washed with brine (500 mL), dried over anhydrous Na₂SO₄, filtered, and concentrated to provide a residue that was used directly.

methyl 4-bromo-2-(1-cyano-2-methylcyclopropyl)benzoate: To a solution of NaH (1.95 g, 48.7 mmol, 60% purity) in DMSO (50 mL) at 0° C. was added methyl 4-bromo-2-(cyanomethyl)benzoate (5.38 g, 21.2 mmol). The mixture was stirred at 20° C. for 1 h. Then 1,2-dibromopropane (4.70 g, 23.3 mmol) was added and the mixture was stirred at 20° C. for 4 h. The mixture was poured into H₂O (10 mL). The mixture was diluted with EtOAc (20 mL) and washed with brine (3×10 mL) and H₂O (10 mL). The combined organic layers were dried over anhydrous Na₂SO₄, filtered, and concentrated under reduced pressure. The residue was purified by silica gel column chromatography. LCMS: m/z=294.0, 296.0 [M+H]⁺.

6′-bromo-2-methyl-2′,3′-dihydro-1′H-spiro[cyclopropane-1,4′-isoquinolin]-1′-one:

To a mixture of methyl 4-bromo-2-(1-cyano-2-methylcyclopropyl)benzoate (1.78 g, 6.05 mmol) and CoCl₂ (785 mg, 6.05 mmol) in MeOH (100 mL) and THF (6 mL) at 0° C. was added NaBH₄ (1.14 g, 30.3 mmol). The mixture was stirred at 20° C. for 3 h. The mixture was diluted with aq. sat. NH₄Cl (100 mL) followed by aq. HCl (1M, 100 mL) and extracted with EtOAc (3×100 mL). The combined organic layers were dried over anhydrous Na₂SO₄, filtered, and concentrated under reduced pressure. The residue was purified by silica gel column chromatography. LCMS: m/z=266.1, 268.1 [M+H]⁺.

methyl 2-(6′-bromo-2-methyl-1′-oxo-1′H-spiro[cyclopropane-1,4′-isoquinolin]-2′(3′H)-yl)acetate: To a solution of 6′-bromo-2-methyl-2′,3′-dihydro-1′H-spiro[cyclopropane-1,4′-isoquinolin]-1′-one (0.56 g, 2.10 mmol) in DMF (10 mL) at 0° C. was added NaH (126 mg, 3.16 mmol, 60% purity). The mixture was stirred at 0° C. for 0.5 h. To the mixture at 0° C. was added methyl 2-bromoacetate (643 mg, 4.21 mmol). The mixture was stirred at 20° C. for 2 h. The mixture was poured into H₂O (10 mL) and extracted with EtOAc (3×10 mL). The combined organic layers were washed with brine (2×10 mL), H₂O (10 mL), dried over anhydrous Na₂SO₄, filtered, and concentrated under reduced pressure. The residue was purified by silica gel column chromatography. LCMS: m/z=338.0, 340.0 [M+H]⁺.

2-(6′-bromo-2-methyl-1′-oxo-1′H-spiro[cyclopropane-1,4′-isoquinolin]-2′(3′H)-yl)-N-(5-fluoropyrimidin-2-yl)acetamide: To a solution of methyl 2-(6′-bromo-2-methyl-1′-oxo-1′H-spiro[cyclopropane-1,4′-isoquinolin]-2′(3′H)-yl)acetate (190 mg, 0.56 mmol) and 5-fluoropyrimidin-2-amine (76 mg, 0.67 mmol) in DCE (6.0 mL) was added AlMe₃ (1 M in n-heptane, 0.84 mL). The mixture was stirred at 80° C. for 4 h. The mixture was quenched with H₂O (10 mL) and extracted with EtOAc (3×10 mL). The combined organic layers were concentrated under reduced pressure. The residue was purified by reverse-phase preparative HPLC and further purified by chiral SFC (Column: Regis(S,S) Whelk-O1, (250 mm×30 mm, 10 μm particle size); Mobile phase: A: CO₂ and B: EtOH; Gradient: B %=25%-50%; Flow rate 70 g/min; detection wavelength: 220 nm; Column temperature: 35° C.; System back pressure: 120 bar) to provide:

2-[(2′s,4s)-6-bromo-2′-methyl-1-oxospiro[3H-isoquinoline-4,1′-cyclopropane]-2-yl]-N-(5-fluoropyrimidin-2-yl)acetamide (first eluting isomer, 279): LCMS: m/z=419.0, 420.9 [M+H]⁺. ¹H NMR (400 MHz, CDCl₃): δ 9.03 (br s, 1H), 8.50 (s, 2H), 8.02 (d, J=8.4 Hz, 1H), 7.53-7.46 (m, 1H), 7.10 (d, J=2.0 Hz, 1H), 4.67 (d, J=16.4 Hz, 1H), 4.53-4.38 (m, 1H), 4.28 (d, J=12.4 Hz, 1H), 2.70 (d, J=12.4 Hz, 1H), 1.46-1.37 (m, 1H), 1.16-1.10 (m, 1H), 1.00 (dd, J=8.8, 6.0 Hz, 1H), 0.85 (d, J=6.4 Hz, 3H).

2-[(2′s,4s)-6-bromo-2′-methyl-1-oxospiro[3H-isoquinoline-4,1′-cyclopropane]-2-yl]-N-(5-fluoropyrimidin-2-yl)acetamide (second eluting isomer, 280): LCMS: m/z=418.9, 421.0 [M+H]⁺, ¹H NMR (400 MHz, CDCl₃): δ 8.91 (br s, 1H), 8.48 (s, 2H), 8.02 (d, J=8.4 Hz, 1H), 7.50 (dd, J=8.4, 2.0 Hz, 1H), 7.10 (d, J=2.0 Hz. 1H), 4.67 (d, J=16.4 Hz, 1H), 4.52-4.38 (m, 1H), 4.28 (d, J=12.4 Hz, 1H), 2.70 (d, J=12.4 Hz, 1H), 1.46-1.36 (m, 1H), 1.16-1.10 (m, 1H), 1.03-0.96 (m, 1H), 0.85 (d, J=6.4 Hz, 3H).

2-[(2′s,4r)-6-bromo-2′-methyl-1-oxospiro[3H-isoquinoline-4,1′-cyclopropane]-2-yl]-N-(5-fluoropyrimidin-2-yl)acetamide (third eluting isomer, 281): LCMS: m/z=418.9, 421.0 [M+H]⁺. ¹H NMR (400 MHz, CDCl₃): δ 9.10 (br s, 1H), 8.49 (s, 2H), 8.01 (d, J=8.4 Hz, 1H), 7.44 (dd, J=8.4, 2.0 Hz, 1H), 6.98 (d, J=2.0 Hz, 1H), 4.76-4.59 (m, 1H), 4.44 (br d, J=16.0 Hz. 1H), 3.87 (d, J=12.8 Hz, 1H), 3.47 (d, J=12.8 Hz, 1H), 1.43-1.36 (m, 1H), 1.28 (d, J=1.6 Hz, 4H), 0.75-0.68 (m, 1H).

2-[(2′s,4r)-6-bromo-2′-methyl-1-oxospiro[3H-isoquinoline-4,1′-cyclopropane]-2-yl]-N-(5-fluoropyrimidin-2-yl)acetamide (fourth eluting isomer, 282): LCMS: m/z=418.9, 420.9 [M+H]⁺. ¹H NMR (400 MHz, CDCl₃): δ 9.13 (br s, 1H), 8.49 (s, 2H), 8.01 (d, J=8.4 Hz, 1H), 7.44 (dd, J=8.4, 2.0 Hz, 1H), 6.98 (d, J=2.0 Hz, 1H), 4.74-4.57 (m, 1H), 4.44 (br d, J=16.0 Hz, 1H), 3.87 (d, J=12.8 Hz, 1H), 3.47 (d, J=12.8 Hz, 1H), 1.43-1.36 (m, 1H), 1.27 (s, 4H), 0.75-0.67 (m, 1H).

### Example 283 and 284

### 2-[(2′s,4r)-6-bromo-2′-chloro-1-oxospiro[3H-isoquinoline-4,1′-cyclopropane]-2-yl]-N-(5-fluoropyrimidin-2-yl)acetamide (283 and 284)

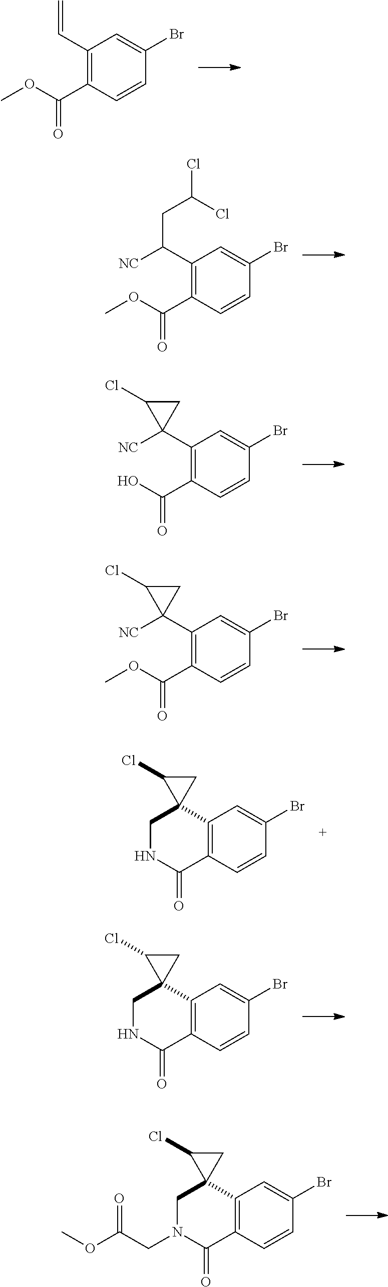

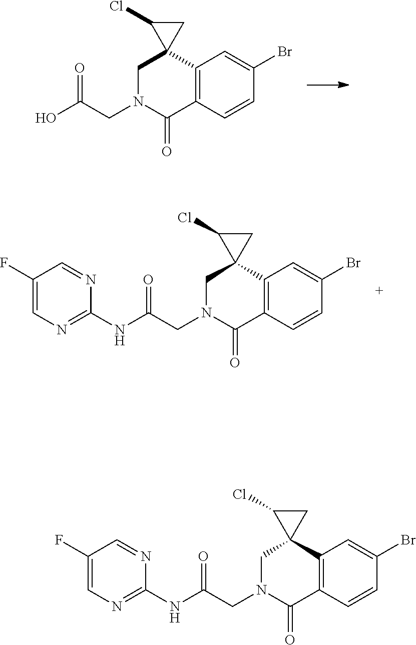

methyl 4-bromo-2-(3,3-dichloro-1-cyanopropyl)benzoate: To a solution of methyl 4-bromo-2-vinylbenzoate (10.0 g, 41.5 mmol) in CHCl₃ (100 mL) and acetone (100 mL) were added tetrakis(acetonitrile)copper(I) hexafluorophosphate (386 mg, 1.04 mmol), TBHP (7.48 g, 83.0 mmol), TMSCN (20.6 g, 207.4 mmol), and DIPEA (26.8 g, 207 mmol). The mixture was degassed by N₂ for 5 min. The mixture was stirred at 40° C. for 24 h. The mixture was concentrated under reduced pressure. The residue was purified by silica gel column chromatography.

methyl 4-bromo-2-(2-chloro-1-cyanocyclopropyl)benzoate: To a solution of methyl 4-bromo-2-(3,3-dichloro-1-cyanopropyl)benzoate (3.6 g, 10.3 mmol) in DMF (30 mL) was added KOH (2.30 g, 41 mmol). The mixture was stirred for 3 h. The mixture was poured into H₂O (20 mL), acidified with 2M HCl (pH=3), and extracted with EtOAc (3×30 mL). The combined organic layers were dried over anhydrous Na₂SO₄, filtered, and concentrated under reduced pressure to provide a residue that was used directly. LCMS: m/z=300.1, 302.1 [M+H]⁺.

methyl 4-bromo-2-(2-chloro-1-cyanocyclopropyl)benzoate: To a solution of 4-bromo-2-(2-chloro-1-cyanocyclopropyl)benzoic acid (3.0 g, 10.0 mmol) in DMF (30 mL) were added K₂CO₃ (2.76 g, 20.0 mmol) and Mel (1.84 g, 13.0 mmol). The mixture was stirred for 16 h. The mixture was diluted with EtOAc (50 mL) and washed with brine (2×50 mL) and H₂O (50 mL). The organics were dried over anhydrous Na₂SO₄, filtered, and concentrated under reduced pressure. The residue was purified by silica gel column chromatography.

(1r,2s)-6′-bromo-2-chloro-2′,3′-dihydro-1′H-spiro[cyclopropane-1,4′-isoquinolin]-1′-one and (1s,2s)-6′-bromo-2-chloro-2′,3′-dihydro-1′H-spiro[cyclopropane-1,4′-isoquinolin]-1′-one: To a mixture of methyl 4-bromo-2-(2-chloro-1-cyanocyclopropyl)benzoate (1.66 g, 5.28 mmol) and CoCl₂ (685 mg, 5.28 mmol) in MeOH (20 mL) at 0° C. was added NaBH₄ (0.55 g, 14.5 mmol). The mixture was stirred at 20° C. for 2 h. The mixture was quenched with aq. sat. NH₄Cl (10 mL), filtered and extracted with EtOAc (3×30 mL). The organic layer was dried over anhydrous Na₂SO₄, filtered, and concentrated under reduced pressure. The residue was purified by silica gel column chromatography to provide:

(1r,2s)-6′-bromo-2-chloro-2′,3′-dihydro-1′H-spiro[cyclopropane-1,4′-isoquinolin]-1′-one: ¹H NMR (400 MHz, CDCl₃): δ 8.00 (d, J=8.4 Hz, 1H), 7.53 (dd, J=8.4, 2.0 Hz, 1H), 6.94 (d, J=2.0 Hz, 1H), 6.26 (br s, 1H), 3.97-3.86 (m, 1H), 3.67-3.57 (m, 1H), 3.27-3.18 (m, 1H), 1.92-1.82 (m, 1H), 1.26-1.24 (m, 1H).

(1s,2s)-6′-bromo-2-chloro-2′,3′-dihydro-1′H-spiro[cyclopropane-1,4′-isoquinolin]-1′-one: ¹H NMR (400 MHz, CDCl₃): δ 8.01 (d, J=8.4 Hz, 1H), 7.58 (dd, J=8.4, 2.0 Hz, 1H), 7.22 (d, J=1.8 Hz, 1H), 7.11-6.97 (m, 1H), 4.04 (d, J=12.8 Hz, 1H), 3.44-3.36 (m, 1H), 2.72 (dd, J=12.8, 5.2 Hz, 1H), 1.73-1.68 (m, 1H), 1.43 (t, J=7.6 Hz, 1H).

methyl 2-((1r,2s)-6′-bromo-2-chloro-1′-oxo-1′H-spiro[cyclopropane-1,4′-isoquinolin]-2′(3′H)-yl)acetate: To a solution of (1r,2s)-6′-bromo-2-chloro-2′,3′-dihydro-1′H-spiro[cyclopropane-1,4′-isoquinolin]-1′-one (335 mg, 1.17 mmol) in DMF (5 mL) at 0° C. was added NaH (70 mg, 1.75 mmol, 60% purity). The mixture was stirred at 0° C. for 0.5 h, then methyl 2-bromoacetate (214 mg, 1.40 mmol) was added and the mixture was stirred at 20° C. for 2 h. The mixture was quenched with H₂O (5 mL) and extracted with EtOAc (10 mL). The combined organic layers were washed with brine (5 mL) and H₂O (5 mL), dried over anhydrous Na₂SO₄, filtered, and concentrated under reduced pressure. The residue was purified by silica gel column chromatography. ¹H NMR (400 MHz, CDCl₃): δ 8.02 (d, J=8.4 Hz, 1H), 7.52 (dd, J=8.4, 2.0 Hz, 1H), 6.92 (d, J=2.0 Hz, 1H), 4.86 (d, J=17.6 Hz, 1H), 4.17 (d, J=12.8 Hz, 1H), 3.89 (d, J=17.6 Hz, 1H), 3.77 (s, 3H), 3.50 (d, J=13.2 Hz, 1H), 3.27-3.19 (m, 1H), 1.89 (t, J=7.6 Hz, 1H), 1.30-1.26 (m, 1H).

2-((1r,2s)-6′-bromo-2-chloro-1′-oxo-1′H-spiro[cyclopropane-1,4′-isoquinolin]-2′(3′H)-yl)acetic acid: To a solution of methyl 2-((1r,2s)-6′-bromo-2-chloro-1′-oxo-1′H-spiro[cyclopropane-1,4′-isoquinolin]-2′(3′H)-yl)acetate (240 mg, 0.67 mmol) in THF (2 mL) and H₂O (2 mL) was added LiOH·H₂O (56 mg, 1.33 mmol). The mixture was stirred for 2 h. The mixture was concentrated and diluted with MTBE (5 mL). The aqueous layer was acidified with 2M HCl (pH=3) and extracted with EtOAc (2×5 mL). The combined organic layers were dried over anhydrous Na₂SO₄, filtered, and concentrated under reduced pressure to provide a residue that was used directly. ¹H NMR (400 MHz, DMSO-d₆): δ 12.79 (br s, 1H), 7.83 (d, J=8.4 Hz, 1H), 7.60 (dd, J=8.4, 2.0 Hz, 1H), 7.34 (d, J=2.0 Hz, 1H), 4.50-4.40 (m, 1H), 3.98 (d, J=17.2 Hz, 1H), 3.93 (d, J=13.2 Hz, 1H), 3.79 (dd, J=7.6, 4.8 Hz, 1H), 3.59 (d, J=13.2 Hz, 1H), 2.05 (t, J=7.6 Hz, 1H), 1.41-1.34 (m, 1H).

2-[(2′r,4s)-6-bromo-2′-chloro-1-oxospiro[3H-isoquinoline-4,1′-cyclopropane]-2-yl]-N-(5-fluoropyrimidin-2-yl)acetamide: To a solution of 2-((1r,2s)-6′-bromo-2-chloro-1′-oxo-1′H-spiro[cyclopropane-1,4′-isoquinolin]-2′(3′H)-yl)acetic acid (100 mg, 0.29 mmol) and 5-fluoropyrimidin-2-amine (82 mg, 0.73 mmol) in pyridine (2 mL) was added EDCI (111 mg, 0.58 mmol). The mixture was stirred at 20° C. for 16 h. The mixture was diluted with EtOAc (10 mL) and washed with H₂O (2×5 mL). The organic layer was dried over anhydrous Na₂SO₄, filtered, and concentrated under reduced pressure. The residue was purified by reverse-phase preparative HPLC. The mixture was further purified by chiral SFC (Column: Regis(s,s) Whelk-O1, (250 mm×30 mm, m particle size); Mobile phase: A: CO₂ and B: EtOH; Gradient: 50% B isocratic; Flow rate: 70 g/min; Detection wavelength: 220 nm; Column temperature: 35° C.; System back pressure: 150 bar) to provide:

2-[(2′r,4s)-6-bromo-2′-chloro-1-oxospiro[3H-isoquinoline-4,1′-cyclopropane]-2-yl]-N-(5-fluoropyrimidin-2-yl)acetamide (first eluting isomer, 283): LCMS: m/z=438.9, 440.9, 442.9 [M+H]⁺. ¹H NMR (400 MHz, CDCl₃): δ 8.86 (br s, 1H), 8.49 (s, 2H), 8.05 (d, J=8.4 Hz, 1H), 7.53 (dd, J=1.6, 8.4 Hz, 1H), 6.94 (d, J=1.6 Hz, 1H), 5.02-4.96 (m, 1H), 4.33-4.22 (m, 2H), 3.61 (d, J=13.2 Hz, 1H), 3.21 (dd, J=5.2, 7.6 Hz, 1H), 1.92 (t, J=7.6 Hz, 1H), 1.34-1.26 (m, 1H).

2-[(2′r,4s)-6-bromo-2′-chloro-1-oxospiro[3H-isoquinoline-4,1′-cyclopropane]-2-yl]-N-(5-fluoropyrimidin-2-yl)acetamide (second eluting isomer, 284): LCMS: m/z=438.9, 440.9, 442.9 [M+H]⁺. ¹H NMR (400 MHz, CDCl₃): δ 8.88 (br s, 1H), 8.49 (s, 2H), 8.05 (d, J=8.4 Hz, 1H), 7.53 (dd, J=1.6, 8.4 Hz, 1H), 6.94 (d, J=1.6 Hz, 1H), 5.03-4.97 (m, 1H), 4.36-4.20 (m, 2H), 3.61 (d, J=13.2 Hz, 1H), 3.21 (dd, J=5.2, 7.6 Hz, 1H), 1.92 (t, J=7.6 Hz, 1H), 1.32-1.28 (m, 1H).

### Example 285

### 2-[(2′s,4r)-6-bromo-2′,8-difluoro-1-oxospiro[3H-isoquinoline-4,1′-cyclopropane]-2-yl]-N-(5-fluoropyrimidin-2-yl)acetamide (285)

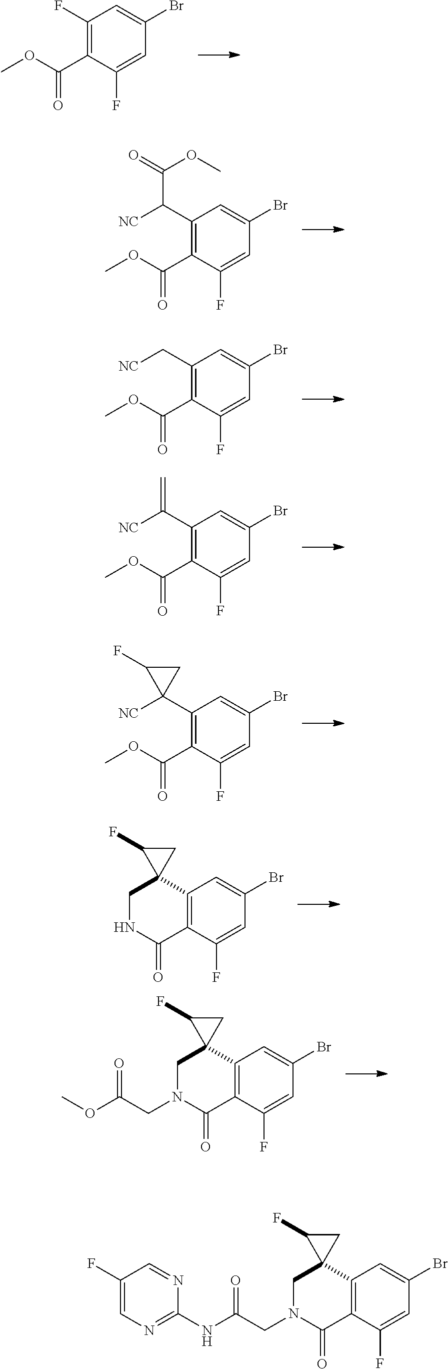

methyl 4-bromo-2-(1-cyano-2-methoxy-2-oxoethyl)-6-fluorobenzoate: To a solution of methyl 4-bromo-2,6-difluorobenzoate (25 g, 0.99 mol) in DMF (250 mL) were added methyl 2-cyanoacetate (9.87 g, 0.99 mol) and Cs₂CO₃ (64.9 g, 1.99 mol). The mixture was stirred at 30° C. for 16 h. The reaction was quenched with brine (500 mL) and aq. HCl (2 M) was added to adjust pH to 2-3. The mixture was extracted with EtOAc (3×500 mL). The combined organic layers were washed with brine (200 mL), dried over anhydrous Na₂SO₄, filtered and concentrated under reduced pressure. LCMS: m/z=330.1, 332.1 [M+H]⁺.

methyl 4-bromo-2-(cyanomethyl)-6-fluorobenzoate: To a solution of methyl 4-bromo-2-(1-cyano-2-methoxy-2-oxoethyl)-6-fluorobenzoate (24 g, 0.73 mol) in DMSO (100 mL) was added brine (100 mL). The mixture was stirred at 130° C. for 16 h. The reaction mixture was poured into brine (300 mL) and extracted with EtOAc (3×300 mL). The combined organic layers were washed with brine (300 mL), dried over anhydrous Na₂SO₄, filtered, and concentrated under reduced pressure. The residue was purified by silica gel column chromatography.

methyl 4-bromo-2-(1-cyanovinyl)-6-fluorobenzoate: To a solution of methyl 4-bromo-2-(cyanomethyl)-6-fluorobenzoate (10 g, 36.8 mol) in DMSO (100 mL) was added N,N,N′,N′-tetramethylmethanediamine (5.63 g, 0.55 mol). To the mixture was added dropwise Ac₂O (12.4 g, 1.21 mol). The mixture was stirred for 2 h. The reaction was quenched with brine (50 mL) and aq. sat. NaHCO₃ was added to adjust pH to 8. The mixture was extracted with EtOAc (3×50 mL). The combined organic layers were washed with brine (50 mL), dried over anhydrous Na₂SO₄, filtered, and concentrated under reduced pressure. The residue was purified by silica gel column chromatography.

methyl 4-bromo-2-(1-cyano-2-fluorocyclopropyl)-6-fluorobenzoate: To a solution of methyl 4-bromo-2-(1-cyanovinyl)-6-fluorobenzoate (4.0 g, 0.14 mol) and (fluoromethyl)(phenyl)(2,3,4,5-tetramethylphenyl)sulfonium tetrafluoroborate (8.16 g, 0.22 mol) in THF (40 mL) at 0° C. was added NaH (2.25 g, 0.56 mol, 60% purity). The mixture was stirred at 20° C. for 4 h. The reaction was quenched with aq. sat. NH₄Cl (150 mL) and extracted with EtOAc (3×150 mL). The combined organic layers were washed with brine (150 mL), dried over anhydrous Na₂SO₄, filtered, and concentrated under reduced pressure. The residue was purified by silica gel column chromatography. LCMS: m/z=315.9, 317.9 [M+H]+

(1r,2s)-6′-bromo-2,8′-difluoro-2′,3′-dihydro-1′H-spiro[cyclopropane-1,4′-isoquinolin]-1′-one: To a solution of methyl 4-bromo-2-(1-cyano-2-fluorocyclopropyl)-6-fluorobenzoate (1.8 g, 50 mmol) in MeOH (20 mL) and H₂O (0.2 mL) at 0° C. were added CoCl₂ (740 mg, 50 mmol) and NaBH₄ (646 mg, 0.17 mol). The mixture was stirred at 0° C. for 2 h. The reaction mixture was poured into ice-cold brine (20 mL) and acidified with HCl (3M) to pH=5. The mixture was extracted with EtOAc (3×100 mL). The combined organic layers were washed with brine (2×100 mL), dried over anhydrous Na₂SO₄, filtered, and concentrated under reduced pressure. The residue was purified by silica gel column chromatography. LCMS: m/z=288.1, 290.1 [M+H]⁺.

methyl 2-((1r,2s)-6′-bromo-2,8′-difluoro-1′-oxo-1′H-spiro[cyclopropane-1,4′-isoquinolin]-2′(3′H)-yl)acetate: To a solution of (1r,2s)-6′-bromo-2,8′-difluoro-2′,3′-dihydro-1′H-spiro[cyclopropane-1,4′-isoquinolin]-1′-one (80 mg, 0.27 mmol) in DMF (8 mL) at 0° C. were added methyl 2-bromoacetate (85 mg, 0.56 mmol) and NaH (17 mg, 0.42 mmol, 60% purity). The mixture was stirred at 20° C. for 2 h. The reaction was quenched with brine (30 mL) and extracted with EtOAc (3×30 mL). The combined organic layers were washed with brine (30 mL), dried over anhydrous Na₂SO₄, filtered, and concentrated under reduced pressure. The residue was purified by silica gel column chromatography. LCMS: m/z=360.2, 362.2 [M+H]⁺.

2-[(2′s,4r)-6-bromo-2′,8-difluoro-1-oxospiro[3H-isoquinoline-4,1′-cyclopropane]-2-yl]-N-(5-fluoropyrimidin-2-yl)acetamide: To a solution of methyl 2-((1r,2s)-6′-bromo-2,8′-difluoro-1′-oxo-1′H-spiro[cyclopropane-1,4′-isoquinolin]-2′(3′H)-yl)acetate (80 mg, 0.22 mmol) in DCE (2 mL) were added 5-fluoropyrimidin-2-amine (30 mg, 0.26 mmol) and AlMe₃ (1 M, 0.33 mL). The mixture was stirred at 80° C. for 10 h. The reaction was quenched with H₂O (6 mL) and extracted with EtOAc (3×6 mL). The combined organic layers were washed with brine (6 mL), dried over anhydrous Na₂SO₄, filtered, and concentrated under reduced pressure. The residue was purified by reverse-phase preparative HPLC. LCMS: m/z=440.8, 442.9 [M+H]⁺. ¹H NMR (400 MHz, CDCl₃): δ 8.55 (br s, 1H), 8.48 (s, 2H), 7.33-7.28 (m, 1H), 6.67 (s, 1H), 5.08-4.88 (m, 1H), 4.74-4.57 (m, 1H), 4.34 (d, J=16.8 Hz, 1H), 4.14 (dd, J=13.2, 2.0 Hz, 1H), 3.50 (d, J=13.2 Hz, 1H), 1.51-1.42 (m, 2H).

### Example 286 and 287

### 2-[(2′s,4r)-6-bromo-2′-cyano-1-oxospiro[3H-isoquinoline-4,1′-cyclopropane]-2-yl]-N-(5-fluoropyrimidin-2-yl)acetamide (286 and 287)

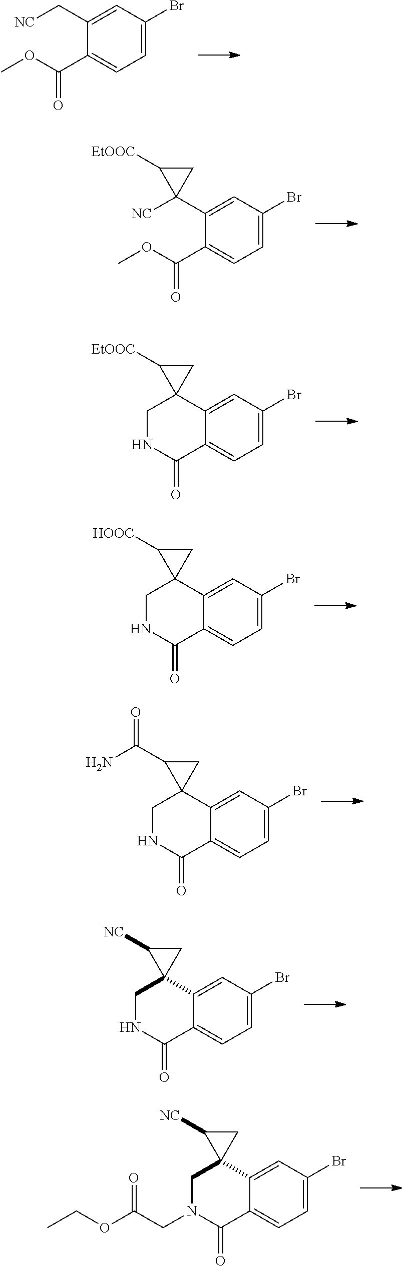

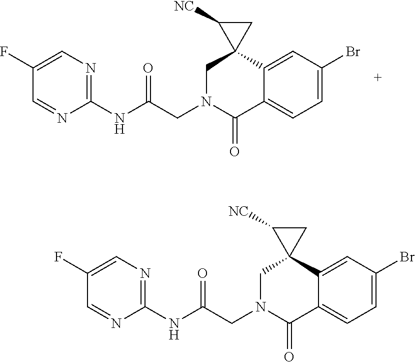 [SMILES: N#C[C@H]1C[C@@]12CN(CC(=O)Nc1ncc(F)cn1)C(=O)c1ccc(Br)cc12 ; N#C[C@@H]1C[C@]12CN(CC(=O)Nc1ncc(F)cn1)C(=O)c1ccc(Br)cc12]

methyl 4-bromo-2-(1-cyano-2-(ethoxycarbonyl)cyclopropyl)benzoate: To a mixture of methyl 4-bromo-2-(cyanomethyl)benzoate (15 g, 59 mmol) and ethyl 2,3-dibromopropanoate (15.4 g, 59 mmol) in THF (10 mL) at 0° C. was added Cs₂CO₃ (58 g, 177 mmol). The mixture was stirred at 70° C. for 2 h. The residue was diluted with H₂O (500 mL) and extracted with EtOAc (3×500 mL). The combined organic layers were washed with brine (500 mL), dried over anhydrous Na₂SO₄, filtered, and concentrated under reduced pressure. The residue was purified by silica gel column chromatography. LCMS: m/z=352.0, 354.0 [M+H]⁺.

ethyl 6′-bromo-1′-oxo-2′,3′-dihydro-1′H-spiro[cyclopropane-1,4′-isoquinoline]-2-carboxylate: To a solution of methyl 4-bromo-2-(1-cyano-2-(ethoxycarbonyl)cyclopropyl)benzoate (9 g, 25.5 mmol) and CoCl₂ (6.64 g, 51.1 mmol) in MeOH (100 mL) and H₂O (10 mL) at 0° C. was added NaBH₄ (5.80 g, 153 mmol). The mixture was stirred at 20° C. for 2 h. The reaction mixture was diluted with aq. sat. NH₄Cl (200 mL) at 0° C., and extracted with DCM (4×200 mL). The combined organic layers were washed with brine (200 mL), dried over anhydrous Na₂SO₄, filtered, and concentrated under reduced pressure. The residue was purified by silica gel column chromatography.

6′-bromo-1′-oxo-2′,3′-dihydro-1′H-spiro[cyclopropane-1,4′-isoquinoline]-2-carboxylic acid: To a mixture of ethyl 6′-bromo-1′-oxo-2′,3′-dihydro-1′H-spiro[cyclopropane-1,4′-isoquinoline]-2-carboxylate (500 mg, 1.54 mmol) in THF (5 mL) and H₂O (1 mL) was added LiOH·H₂O (129 mg, 3.08 mmol). The mixture was stirred at 25° C. for 16 h. The reaction mixture was concentrated under reduced pressure. The residue was diluted with H₂O (2 mL) and adjusted pH=4 with aq. HCl (3 M). The mixture was filtered and the collected solid was used directly. LCMS: m/z=295.9, 297.9 [M+H]⁺.

6′-bromo-1′-oxo-2′,3′-dihydro-1′H-spiro[cyclopropane-1,4′-isoquinoline]-2-carboxamide: To a mixture of 6′-bromo-1′-oxo-2′,3′-dihydro-1′H-spiro[cyclopropane-1,4′-isoquinoline]-2-carboxylic acid (100 mg, 0.34 mmol) and NH₄Cl (36 mg, 0.68 mmol) in DCM (2 mL) at 0° C. were added HOBt (55 mg, 0.405 mmol), DIPEA (87 mg, 0.68 mmol), and EDCI (78 mg, 0.41 mmol). The mixture was stirred at 25° C. for 16 h. The reaction mixture was diluted with H₂O (10 mL) and extracted with DCM:MeOH (v:v=10:1, 3×10 mL). The combined organic layers were washed with brine (3×10 mL), dried over anhydrous Na₂SO₄, filtered, and concentrated under reduced pressure. The residue was purified by preparative silica gel thin-layer chromatography. LCMS: m/z=295.0, 296.9 [M+H]⁺.

(1r,2s)-6′-bromo-1′-oxo-2′,3′-dihydro-1′H-spiro[cyclopropane-1,4′-isoquinoline]-2-carbonitrile: To a mixture of 6′-bromo-1′-oxo-2′,3′-dihydro-1′H-spiro[cyclopropane-1,4′-isoquinoline]-2-carboxamide (2.4 g, 8.13 mmol) in THF (30 mL) at 0° C. were added Et₃N (2.47 g, 24.4 mmol) and TFAA (3.42 g, 16.3 mmol). The mixture was stirred at 0° C. for 2 h. The reaction mixture was quenched by addition of aq. sat. NaHCO₃ (30 mL) and extracted with DCM (3×20 mL). The combined organic layers were washed with brine (20 mL), dried over anhydrous Na₂SO₄, filtered, and concentrated under reduced pressure to provide a residue that was used directly. LCMS: m/z=277.0, 279.0 [M+H]⁺.

ethyl 2-((1r,2s)-6′-bromo-2-cyano-1′-oxo-1′H-spiro[cyclopropane-1,4′-isoquinolin]-2′(3′H)-yl)acetate: To a mixture of ethyl 2-iodoacetate (59 mg, 0.28 mmol) and (1r,2s)-6′-bromo-1′-oxo-2′,3′-dihydro-1′H-spiro[cyclopropane-1,4′-isoquinoline]-2-carbonitrile (70 mg, 0.25 mmol) in DMF (3 mL) was added Cs₂CO₃ (123 mg, 0.38 mmol). The mixture was stirred at 20° C. for 3 h. The reaction mixture was quenched by addition of H₂O (10 mL) and extracted with EtOAc (3×5 mL). The combined organic layers were washed with brine (10 mL), dried over anhydrous Na₂SO₄, filtered, and concentrated under reduced pressure to provide a residue that was used directly. LCMS: m/z=362.9, 364.9 [M+H]⁺.

2-[(2′s,4r)-6-bromo-2′-cyano-1-oxospiro[3H-isoquinoline-4,1′-cyclopropane]-2-yl]-N-(5-fluoropyrimidin-2-yl)acetamide: To a mixture of ethyl 2-((1r,2s)-6′-bromo-2-cyano-1′-oxo-1′H-spiro[cyclopropane-1,4′-isoquinolin]-2′(3′H)-yl)acetate (70 mg, 0.19 mmol) and 5-fluoropyrimidin-2-amine (65 mg, 0.58 mmol) in DCE (1.0 mL) was added AlMe₃ (2.0 M in heptane, 0.3 mL). The mixture was stirred at 80° C. for 12 h. The reaction mixture was diluted with H₂O (10 mL) and extracted with EtOAc (3×5 mL). The combined organic layers were washed with brine (5 mL), dried over anhydrous Na₂SO₄, filtered, and concentrated under reduced pressure. The residue was purified by reverse-phase preparative HPLC. The mixture was further purified by chiral SFC (Column: Regis(S,S) Whelk-O1 (250 mm×30 mm, 10 μm particle size); Mobile phase: A: CO₂ and B: 0.1% NH₄OH in EtOH; Gradient: 50% B isocratic; Row rate: 70 g/min; Wavelength: 220 nm; Column temperature: 35° C.; System back pressure: 150 bar) to provide:

2-[(2′s,4r)-6-bromo-2′-cyano-1-oxospiro[3H-isoquinoline-4,1′-cyclopropane]-2-yl]-N-(5-fluoropyrimidin-2-yl)acetamide (first eluting isomer, 286): LCMS: m/z=430.0, 432.0 [M+H]⁺. ¹H NMR (400 MHz, CDCl₃): δ 8.63 (br s, 1H), 8.48 (s, 2H), 8.08 (d, J=8.4 Hz, 1H), 7.58 (dd, J=1.2, 8.4 Hz, 1H), 6.99 (d, J=1.2 Hz. 1H), 5.44 (br d, J=16.8 Hz, 1H), 4.41-4.21 (m, 2H), 3.43 (d, J=13.2 Hz, 1H), 1.99-1.97 (m, 1H), 1.77-1.75 (m, 1H), 1.64-1.61 (m, 1H).

2-[(2′s,4r)-6-bromo-2′-cyano-1-oxospiro[3H-isoquinoline-4,1′-cyclopropane]-2-yl]-N-(5-fluoropyrimidin-2-yl)acetamide (second eluting isomer, 287): LCMS: m/z=429.9, 431.9 [M+H]⁺. ¹H NMR (400 MHz, CDCl₃): δ 8.69 (br s, 1H), 8.49 (s, 2H), 8.08 (d, J=8.4 Hz, 1H), 7.58 (br d, J=8.4 Hz, 1H), 6.99 (s, 1H), 5.53-5.34 (m, 1H), 4.41-4.23 (m, 2H), 3.43 (d, J=13.2 Hz, 1H), 2.03-1.96 (m, 1H), 1.78-1.72 (m, 1H), 1.65-1.62 (m, 1H).

### Example 288 and 289

### N-(5-fluoropyrimidin-2-yl)-2-[6-bromo-1′,1′,5-trifluoro-1-oxospiro[3H-isoquinoline-4,2′-cyclopropane]-2-yl]acetamide (288 and 289)

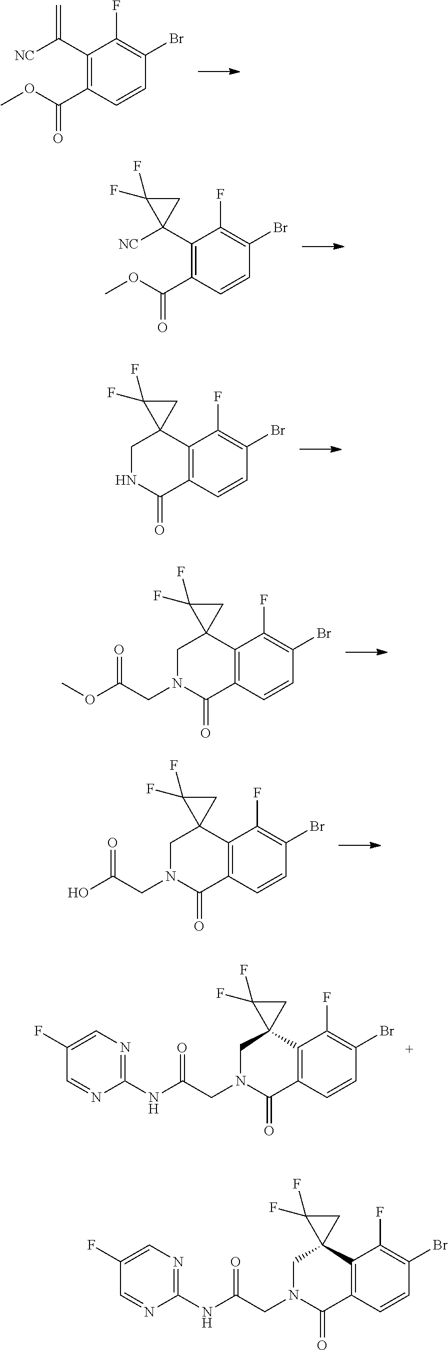

methyl 4-bromo-2-(1-cyano-2,2-difluorocyclopropyl)-3-fluorobenzoate: To a solution of methyl 4-bromo-2-(1-cyanovinyl)-3-fluorobenzoate (450 mg, 1.58 mmol, Int. 36) in 1,4-dioxane (2.0 mL) was added sodium 2-chloro-2,2-difluoroacetate (725 mg. 4.75 mmol). The mixture was stirred at 150° C. for 20 min under microwave irradiation. The reaction mixture was poured into H₂O (20 mL) and extracted with EtOAc (4×15 mL). The combined organic layers were washed with brine (2×10 mL), dried over anhydrous Na₂SO₄, filtered, concentrated under reduced pressure and purified by reverse-phase preparative HPLC. LCMS: m/z=333.9, 335.9 [M+H]⁺.

6′-bromo-2,2,5′-trifluoro-2′,3′-dihydro-1′H-spiro[cyclopropane-1,4′-isoquinolin]-1′-one: To a mixture of methyl 4-bromo-2-(1-cyano-2,2-difluorocyclopropyl)-3-fluorobenzoate (600 mg, 1.80 mmol) and CoCl₂ (233 mg, 1.80 mmol) in MeOH (12 mL) and H₂O (1.2 mL) at −10° C. was added NaBH₄ (204 mg, 5.39 mmol). The mixture was stirred at 0° C. for 1 h and then −10° C. for a further 4 h. The reaction mixture was diluted with sat. aq. NH₄Cl(20 mL) and extracted with EtOAc (4×15 mL). The combined organic layers were washed with brine (2×10 mL), dried over anhydrous Na₂SO₄, filtered, concentrated under reduced pressure, and purified by silica gel column chromatography. LCMS: m/z=306.0, 308.0 [M+H]⁺.

methyl 2-[6-bromo-1′,1′,5-trifluoro-1-oxospiro[3H-isoquinoline-4,2′-cyclopropane]-2-yl]acetate: To a solution of methyl 2-bromoacetate (82 mg, 0.54 mmol) in DMF (1.5 mL) were added 6′-bromo-2,2,5′-trifluoro-2′,3′-dihydro-1′H-spiro[cyclopropane-1,4′-isoquinolin]-1′-one (110 mg, 0.36 mmol), Cs₂CO₃ (234 mg, 0.72 mmol), and NaI (27 mg, 0.18 mmol). The mixture was stirred at 20° C. for 2 h. The reaction mixture was diluted with H₂O (10 mL) at 0° C., and extracted with EtOAc (4×5 mL). The combined organic layers were washed with brine (10 mL), dried over anhydrous Na₂SO₄, filtered, concentrated under reduced pressure, and purified by silica gel column chromatography. LCMS: m/z=378.0, 380.0 [M+H]⁺.

2-[6-bromo-1′,1′,5-trifluoro-1-oxospiro[3H-isoquinoline-4,2′-cyclopropane]-2-yl]acetic acid: To a solution of methyl 2-[6-bromo-1′,1′,5-trifluoro-1-oxospiro[3H-isoquinoline-4,2′-cyclopropane]-2-yl]acetate (115 mg, 0.30 mmol) in THF (3.0 mL) and H₂O (0.6 mL) was added LiOH·H₂O (26 mg, 0.61 mmol). The mixture was stirred at 20° C. for 1 h. The reaction mixture was poured into H₂O (10 mL) and washed with MTBE (3×5 mL). The aqueous layer was adjusted to pH=3 with aq. HCl (3 M) at 0° C., and extracted with EtOAc (3×10 mL). The combined organic layers were washed with brine (10 mL), dried over anhydrous Na₂SO₄, filtered, and concentrated under reduced pressure to provide a residue that was used directly. LCMS: m/z=361.9, 363.9 [M−H]⁻.

N-(5-fluoropyrimidin-2-yl)-2-[6-bromo-1′,1′,5-trifluoro-1-oxospiro[3H-isoquinoline-4,2′-cyclopropane]-2-yl]acetamide: To a solution of 2-[6-bromo-1′,1′,5-trifluoro-1-oxospiro[3H-isoquinoline-4,2′-cyclopropane]-2-yl]acetic acid (85 mg, 0.23 mmol) in pyridine (2.0 mL) were added 5-fluoropyrimidin-2-amine (53 mg, 0.47 mmol) and EDCI (134 mg, 0.70 mmol). The mixture was stirred for 1 h. The mixture was diluted with H₂O (10 mL) and extracted with EtOAc (3×5 mL). The combined organic layers were washed with brine (10 mL), dried over anhydrous Na₂SO₄, filtered, and concentrated under reduced pressure. The residue was purified by reverse-phase preparative HPLC and further purified by chiral SFC (column: Regis(S,S) Whelk-O1 (250 mm×25 mm, 10 m particle size); Mobile phase: A: CO₂ B: 0.1% NH₄OH in i-PrOH; Gradient: B %: 50% isocratic; Flow rate: 3.4 mL/min; Detection Wavelength: 220 nm; Column temperature: 35° C.; System back pressure: 124 bar) to provide:

N-(5-fluoropyrimidin-2-yl)-2-[6-bromo-1′,1′,5-trifluoro-1-oxospiro[3H-isoquinoline-4,2′-cyclopropane]-2-yl]acetamide (first eluting isomer, 288): LCMS: m/z=458.9, 460.9 [M+H]⁺. ¹H NMR (400 MHz, CDCl₃): δ 8.78 (br s, 1H), 8.48 (s, 2H), 7.92 (d, J=8.4 Hz, 1H), 7.64 (dd, J=6.4, 8.4 Hz, 1H), 5.37-5.08 (m, 1H), 4.46 (dd, J=6.8, 13.2 Hz, 1H), 4.23 (d, J=17.2 Hz, 1H), 3.12 (br d, J=12.4 Hz, 1H), 3.01-2.87 (m, 1H), 1.69-1.64 (m, 1H).

N-(5-fluoropyrimidin-2-yl)-2-[6-bromo-1′,1′,5-trifluoro-1-oxospiro[3H-isoquinoline-4,2′-cyclopropane]-2-yl]acetamide (second eluting isomer, 289): LCMS: m/z=458.9, 460.9 [M+H]⁺. ¹H NMR (400 MHz, DMSO-d₆): δ 10.99 (s, 1H), 8.75 (s, 2H), 7.85 (dd, J=6.4, 8.4 Hz, 1H), 7.77 (d, J=8.4 Hz, 1H), 4.83 (br d, J=17.2 Hz, 1H), 4.29 (dd, J=7.2, 13.6 Hz, 1H), 4.19 (d, J=17.2 Hz, 1H), 3.29 (d, J=13.6 Hz, 1H), 2.87-2.77 (m, 1H), 2.11-2.00 (m, 1H).

### Example 290 and 291

### 2-[6-bromo-1′,1′-difluoro-1-oxospiro[3H-isoquinoline-4,2′-cyclopropane]-2-yl]-N-(5-fluoropyrimidin-2-yl)acetamide (290 and 291)

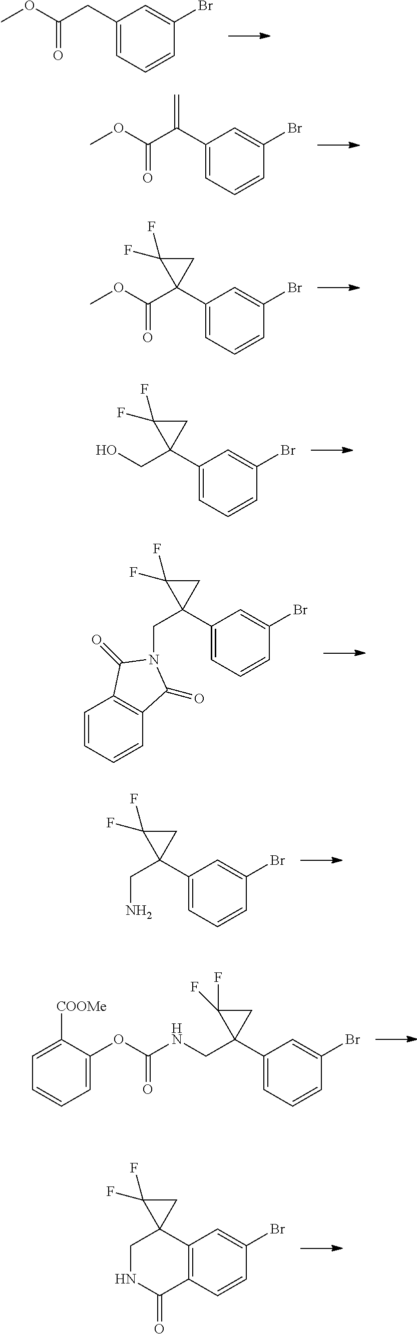

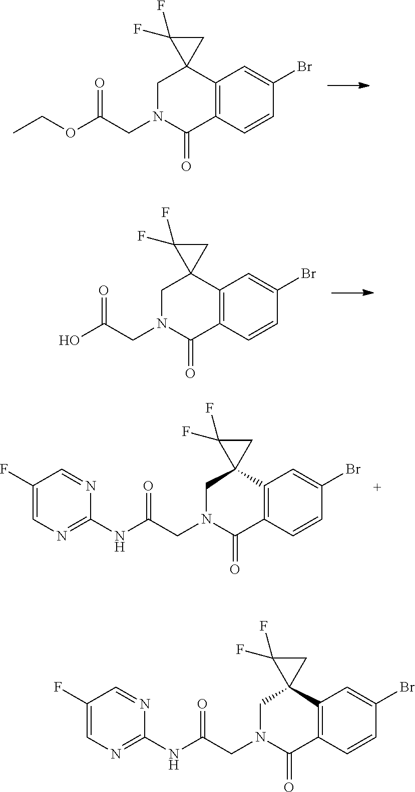

methyl 2-(3-bromophenyl)acrylate: To a solution of methyl 2-(3-bromophenyl)acetate (20 g, 87.3 mmol) in THF (150 mL) were added K₂CO₃ (36.2 g, 262 mmol) and formaldehyde (26.2 g, 873 mmol). The mixture was stirred at 80° C. for 2 h. The mixture was filtered through a pad of celite and the filtrate was concentrated under reduced pressure. The residue was purified by silica gel column chromatography. LCMS: m/z=241.0, 243.0 [M+H]⁺.

methyl 1-(3-bromophenyl)-2,2-difluorocyclopropanecarboxylate: To a solution of methyl 2-(3-bromophenyl)acrylate (9.55 g, 39.6 mmol) in THF (100 mL) was added NaI (2.97 g, 19.8 mmol). To the mixture was added dropwise TMSCF₃ (11.3 g, 79 mmol) at 80° C. The mixture was stirred at 80° C. for 1 h. The mixture was diluted with MTBE (30 mL) and filtered. The filtrate was concentrated under reduced pressure to provide a residue that was used directly. LCMS: m/z=290.9, 292.9 [M+H]⁺.

(1-(3-bromophenyl)-2,2-difluorocyclopropyl)methanol: To a solution of methyl 1-(3-bromophenyl)-2,2-difluorocyclopropanecarboxylate (6.0 g, 20.6 mmol) in DCM (60 mL) at −20° C. was added DIBAL-H (1 M in toluene, 30.9 mL). The mixture was stirred at 0° C. for 1 h. The mixture was poured into aq. HCl (50 mL, 1M) and extracted with DCM (3×20 mL). The combined organic layers were washed with brine (30 mL), dried over anhydrous Na₂SO₄, filtered, and concentrated under reduced pressure. The residue was purified by silica gel column chromatography.

2-((1-(3-bromophenyl)-2,2-difluorocyclopropyl)methyl)isoindoline-1,3-dione: To a solution of (1-(3-bromophenyl)-2,2-difluorocyclopropyl)methanol (4.9 g, 18.6 mmol) in THF (40 mL) were added phthalimide (5.48 g, 37.3 mmol), PPh₃ (9.77 g, 37.3 mmol), and DIAD (7.53 g, 37.3 mmol). The mixture was stirred at 50° C. for 16 h. The mixture was diluted with H₂O (50 mL) and extracted with EtOAc (3×20 mL). The combined organic layers were washed with brine (40 mL), dried over anhydrous Na₂SO₄, filtered, and concentrated under reduced pressure. The residue was purified by silica gel column chromatography.

(1-(3-bromophenyl)-2,2-difluorocyclopropyl)methanamine: To a solution of 2-((1-(3-bromophenyl)-2,2-difluorocyclopropyl)methyl)isoindoline-1,3-dione (7.3 g, 18.6 mmol) in EtOH (70 mL) was added NH₂NH₂·H₂O (9.32 g, 186 mmol). The mixture was stirred at 50° C. for 6 h. The mixture was acidified with aq. HCl (3 M) to pH=3 and washed with EtOAc (2×30 mL). The aqueous phase was adjusted to pH=8 by addition of sat. aq. NaHCO₃ and extracted with EtOAc (3×40 mL). The combined organic layers were washed with brine (70 mL), dried over anhydrous Na₂SO₄, filtered, and concentrated under reduced pressure to provide a residue that was used directly. LCMS: m/z=262.0, 264.0 [M+H]⁺.

methyl 2-((((1-(3-bromophenyl)-2,2-difluorocyclopropyl)methyl)carbamoyl)oxy)benzoate: A solution of (1-(3-bromophenyl)-2,2-difluorocyclopropyl)methanamine (4.8 g, 18.3 mmol) and dimethyl 2,2′-(carbonylbis(oxy))dibenzoate (5.04 g, 15.3 mmol) in THF (50 mL) was stirred at 16 h. The mixture was diluted with H₂O (8 mL) and extracted with EtOAc (3×3 mL). The combined organic layers were washed with brine (5 mL), dried over anhydrous Na₂SO₄, filtered, and concentrated under reduced pressure. The residue was purified by reverse-phase preparative HPLC. LCMS: m/z=440.0, 442.0 [M+H]⁺.

6′-bromo-2,2-difluoro-2′,3′-dihydro-1′H-spiro[cyclopropane-1,4′-isoquinolin]-1′-one: To a solution of methyl 2-((((1-(3-bromophenyl)-2,2-difluorocyclopropyl)methyl)carbamoyl)oxy)benzoate (2.0 g, 4.54 mmol) in DCM (20 mL) at 0° C. was added TfOH (13.6 g, 90.9 mmol). The mixture was stirred at 0° C. for 1 h. The mixture was poured into H₂O (20 mL) and extracted with DCM (3×8 mL). The combined organic layers were washed with brine (15 mL), dried over anhydrous Na₂SO₄, filtered, and concentrated under reduced pressure. The residue was purified by silica gel column chromatography. LCMS: m/z=288.0, 290.0 [M+H]⁺.

ethyl 2-(6′-bromo-2,2-difluoro-1′-oxo-1′H-spiro[cyclopropane-1,4′-isoquinolin]-2′(3′H)-yl)acetate: To a solution of 6′-bromo-2,2-difluoro-2′,3′-dihydro-1′H-spiro[cyclopropane-1,4′-isoquinolin]-1′-one (1.0 g, 3.47 mmol) in DMF (10 mL) was added Cs₂CO₃ (2.26 g, 6.94 mmol). The mixture was cooled to 0° C., and ethyl 2-iodoacetate (1.11 g, 5.21 mmol) was added. The mixture was stirred at 20° C. for 2 h. The mixture was quenched by sat. aq. NH₄Cl (10 mL) and extracted with EtOAc (3×5 mL). The combined organic layers were washed with brine (15 mL), dried over anhydrous Na₂SO₄, filtered, and concentrated under reduced pressure. The residue was purified by reverse-phase preparative HPLC. LCMS: m/z=374.1, 376.0 [M+H]⁺.

2-(6′-bromo-2,2-difluoro-1′-oxo-1′H-spiro[cyclopropane-1,4′-isoquinolin]-2′(3′H)-yl)acetic acid: To a solution of ethyl 2-(6′-bromo-2,2-difluoro-1′-oxo-1′H-spiro[cyclopropane-1,4′-isoquinolin]-2′(3′H)-yl)acetate (55 mg, 0.15 mmol) in THF (1.0 mL) and H₂O (1.0 mL) was added LiOH·H₂O (15 mg, 0.37 mmol). The mixture was stirred for 1 h at 25° C. The mixture was diluted with water (3 mL) and washed with MTBE (2 mL). The aqueous phase was adjusted pH=3 with HCl (3 M) and the mixture was extracted with EtOAc (3×1 mL). The combined organic layers were washed with brine (4 mL), dried over anhydrous Na₂SO₄, filtered, and concentrated under reduced pressure to provide a residue that was used directly. LCMS: m/z=346.0, 348.0 [M+H]⁺.

2-[6-bromo-1′,1′-difluoro-1-oxospiro[3H-isoquinoline-4,2′-cyclopropane]-2-yl]-N-(5-fluoropyrimidin-2-yl)acetamide: To a solution of 2-(6′-bromo-2,2-difluoro-1′-oxo-1′H-spiro[cyclopropane-1,4′-isoquinolin]-2′(3′H)-yl)acetic acid (30 mg, 0.09 mmol) in pyridine (1.0 mL) was added 5-fluoropyrimidin-2-amine (20 mg, 0.17 mmol) and EDCI (50 mg, 0.26 mmol). The mixture was stirred for 1 h. The mixture was diluted with H₂O (2 mL) and extracted with EtOAc (3×1 mL). The combined organic layers were washed with brine (2 mL), dried over anhydrous Na₂SO₄, filtered, and concentrated under reduced pressure. The residue was purified by reverse-phase preparative HPLC and further purified by chiral SFC (Column: Regis(S,S) Whelk-O1 (250 mm×30 mm, 10 μm particle size); Mobile phase: A: CO₂ and B: EtOH; Gradient: 50% B isocratic; Flow rate: 80 g/min; Detection wavelength: 220 nm; Column temperature: 40° C.; System back pressure: 100 bar) to provide:

2-[6-bromo-1′,1′-difluoro-1-oxospiro[3H-isoquinoline-4,2′-cyclopropane]-2-yl]-N-(5-fluoropyrimidin-2-yl)acetamide (first eluting isomer, 290): LCMS: m/z=440.9, 442.9 [M+H]⁺. ¹H NMR (400 MHz, CDCl₃): δ 8.87 (br s, 1H), 8.49 (s, 2H), 8.07 (d, J=8.4 Hz, 1H), 7.58 (d, J=8.4 Hz, 1H), 7.23 (s, 1H), 5.13 (br d, J=15.8 Hz, 1H), 4.31 (br d, J=16.8 Hz, 1H), 4.19 (dd, J=5.6, 12.8 Hz, 1H), 3.43 (br d, J=12.8 Hz, 1H), 2.11-2.05 (m, 1H), 1.75-1.71 (m, 1H).

2-[6-bromo-1′,1′-difluoro-1-oxospiro[3H-isoquinoline-4,2′-cyclopropane]-2-yl]-N-(5-fluoropyrimidin-2-yl)acetamide (second eluting isomer, 291): LCMS: m/z=440.9, 442.9 [M+H]⁺. ¹H NMR (400 MHz, CDCl₃): δ 8.96 (br s, 1H), 8.49 (s, 2H), 8.07 (d, J=8.4 Hz, 1H), 7.58 (dd, J=1.6, 8.4 Hz, 1H), 7.23 (s, 1H), 5.14 (br d, J 15.8 Hz, 1H), 4.31 (br d, J 16.8 Hz, 1H), 4.19 (dd, J=5.6, 12.8 Hz, 1H), 3.43 (br d, J=12.8 Hz, 1H), 2.11-2.05 (m, 1H), 1.75-1.71 (m, 1H).

### Example 292 and 293

### 2-[6-bromo-4-(fluoromethyl)-1-oxo-3,4-dihydroisoquinolin-2-yl]-N-(5-fluoropyrimidin-2-yl)acetamide (292 and 293)

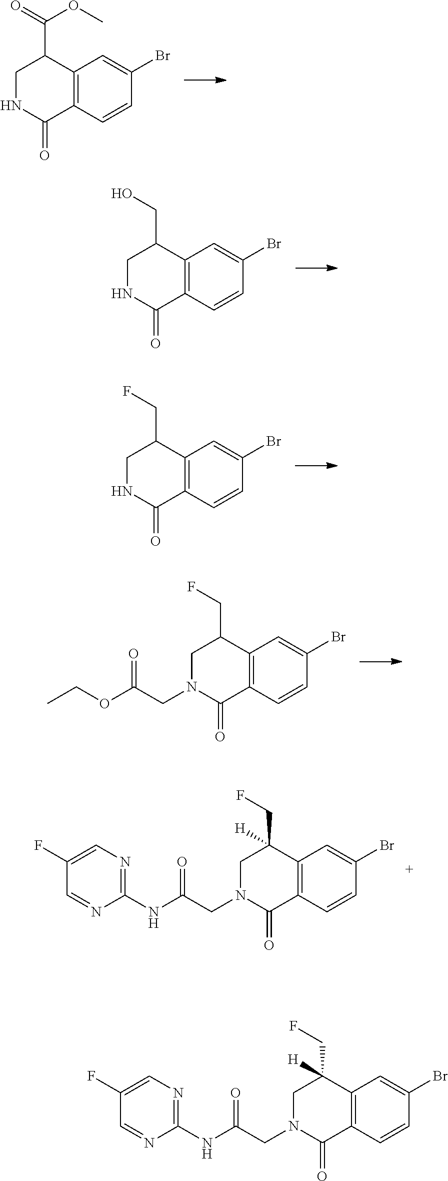 [SMILES: COC(=O)C1CNC(=O)c2ccc(Br)cc21 ; O=C1NCC(CO)c2cc(Br)ccc21 ; O=C1NCC(CF)c2cc(Br)ccc21 ; CCOC(=O)CN1CC(CF)c2cc(Br)ccc2C1=O ; O=C(CN1C[C@@H](CF)c2cc(Br)ccc2C1=O)Nc1ncc(F)cn1 ; O=C(CN1C[C@H](CF)c2cc(Br)ccc2C1=O)Nc1ncc(F)cn1]

6-bromo-4-(hydroxymethyl)-3,4-dihydroisoquinolin-1(2H)-one: To a solution of methyl 6-bromo-1-oxo-1,2,3,4-tetrahydroisoquinoline-4-carboxylate (1.25 g, 4.40 mmol, Int. 60) in THF (5 mL) at 0° C. was added LiBH₄ (192 mg, 8.80 mmol). The mixture was stirred at 15° C. for 5 h. The mixture was poured into ice-cold H₂O (15 mL) and extracted with DCM (3×20 mL). The combined organic layers were washed with brine (30 mL), dried over anhydrous Na₂SO₄, filtered, and concentrated under reduced pressure to provide a residue that was used directly. ¹H NMR (400 MHz, CDCl₃): δ 7.94 (br d, J=8.2 Hz, 1H), 7.54 (br d, J=8.0 Hz, 1H), 7.46 (s, 1H), 6.23 (br s, 1H), 3.76-3.86 (m, 2H), 3.66-3.75 (m, 2H), 3.00-3.15 (m, 1H), 2.19-2.34 (m, 1H).

6-bromo-4-(fluoromethyl)-3,4-dihydroisoquinolin-1(2H)-one: To a solution of 6-bromo-4-(hydroxymethyl)-3,4-dihydroisoquinolin-1(2H)-one (800 mg, 3.12 mmol) in DCM (10 mL) at −78° C. was added N,N-diethylethanamine;trihydrofluoride (1.51 g, 9.37 mmol) and (difluoro-λ⁴-sulfanylidene)-diethyl-ammonium;tetrafluoroborate (1.43 g, 6.25 mmol). The mixture was stirred at 15° C. for 16 h. The mixture was poured into H₂O (10 mL) and extracted with DCM (3×5 mL). The combined organic layers were washed with brine (10 mL), dried over anhydrous Na₂SO₄, filtered, and concentrated under reduced pressure. The crude material was triturated with MTBE (50 mL) to provide a residue that was used directly. LCMS: m/z: 258.0, 260.0 [M+H]⁺.

ethyl 2-(6-bromo-4-(fluoromethyl)-1-oxo-3,4-dihydroisoquinolin-2(1H)-yl)acetate: To a solution of 6-bromo-4-(fluoromethyl)-3,4-dihydro-2H-isoquinolin-1-one (260 mg, 1.01 mmol) in DMF (5.0 mL) were added Cs₂CO₃ (492 mg, 1.51 mmol) and ethyl 2-iodoacetate (259 mg, 1.21 mmol). The mixture was stirred for 16 h at 25° C. The mixture was poured into ice-cold H₂O (10 mL) and extracted with EtOAc (3×15 mL). The combined organic layers were washed with brine (30 mL), dried over anhydrous Na₂SO₄, filtered, concentrated under reduced pressure, and purified by silica gel column chromatography. LCMS: m/z=344.0, 346.0 [M+H]⁺.

2-[6-bromo-4-(fluoromethyl)-1-oxo-3,4-dihydroisoquinolin-2-yl]-N-(5-fluoropyrimidin-2-yl)acetamide: To a solution of ethyl 2-(6-bromo-4-(fluoromethyl)-1-oxo-3,4-dihydroisoquinolin-2(1H)-yl)acetate (300 mg, 0.87 mmol) and 5-fluoropyrimidin-2-amine (118 mg, 1.05 mmol) in DCE (1.5 mL) was added AlMe₃ (1 M in n-heptane, 0.65 mL). The mixture was stirred at 60° C. for 16 h. The reaction mixture was washed with H₂O (15 mL), filtered, and extracted with EtOAc (3×10 mL). The combined organic layers were dried over anhydrous Na₂SO₄, filtered, and concentrated under reduced pressure. The residue was purified by reverse-phase preparative HPLC. The mixture was purified further by chiral SFC (Column: Regis(S,S) Whelk-O1 (250 mm×25 mm, 10 μm particle size); Mobile phase: A: CO₂ and B: 0.1% i-PrNH₂ in i-PrOH; Gradient: 45% B isocratic; Flow rate: 3.4 mL/min; Detection wavelength: 220 nm; Column temperature: 35° C.; System back pressure: 124 bar) to provide:

2-[6-bromo-4-(fluoromethyl)-1-oxo-3,4-dihydroisoquinolin-2-yl]-N-(5-fluoropyrimidin-2-yl)acetamide (first eluting isomer, 292): LCMS: m/z=410.9, 413.0 [M+H]⁺. ¹H NMR (400 MHz, CDCl₃): δ 8.66 (br s, 1H), 8.48 (s, 2H), 8.02 (d, J=8.2 Hz, 1H), 7.62-7.54 (m, 1H), 7.46 (s, 1H), 4.85-4.46 (m, 4H), 4.10-4.03 (m, 1H), 3.75-3.66 (m, 1H), 3.40-3.30 (m, 1H).

2-[6-bromo-4-(fluoromethyl)-1-oxo-3,4-dihydroisoquinolin-2-yl]-N-(5-fluoropyrimidin-2-yl)acetamide (second eluting isomer, 293): LCMS: m/z=411.0, 412.9 [M+H]⁺. ¹H NMR (400 MHz, CDCl₃): δ 8.88 (br s, 1H), 8.48 (s, 2H), 8.02 (d, J=8.2 Hz, 1H), 7.57 (dd, J=8.2, 1.6 Hz, 1H), 7.45 (d, J=1.2 Hz, 1H), 4.88-4.45 (m, 4H), 4.06 (br dd, J=12.4, 2.0 Hz, 1H), 3.71 (dd, J=12.8, 2.4 Hz, 1H), 3.42-3.29 (m, 1H).

### Example 294

### 2-[(4r)-6-bromo-4-[(1r)-1-fluoroethyl]-1-oxo-3,4-dihydroisoquinolin-2-yl]-N-(5-fluoropyrimidin-2-yl)acetamide (294)

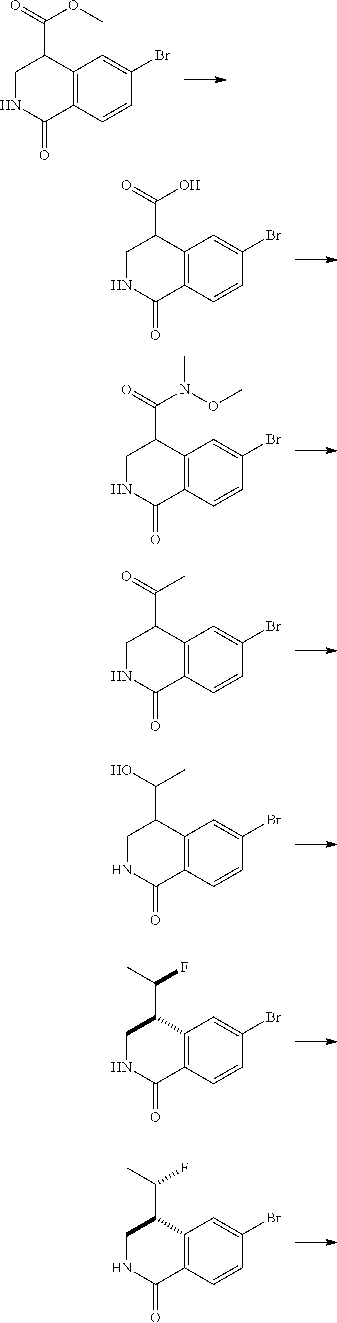

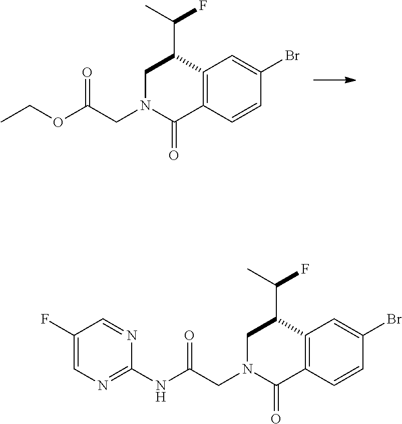

6-bromo-1-oxo-1,2,3,4-tetrahydroisoquinoline-4-carboxylic acid: To a solution of methyl 6-bromo-1-oxo-1,2,3,4-tetrahydroisoquinoline-4-carboxylate (20.0 g, 71.0 mmol, Int. 60) in THF (200 mL) and H₂O (200 mL) was added LiOH·H₂O (7.43 g, 177 mmol). The mixture was stirred for 16 h. The reaction mixture was washed with EtOAc (200 mL). The aqueous phase was diluted with HCl (3 M) to pH=3 and extracted with EtOAc (3×200 mL). The combined organic layers were washed with brine (200 mL), dried over anhydrous Na₂SO₄, filtered, and concentrated under reduced pressure to provide a residue that was used directly.

### 6-bromo-N-methoxy-N-methyl-1-oxo-1,2,3,4-tetrahydroisoquinoline-4-carboxamide

To a solution of 6-bromo-1-oxo-1,2,3,4-tetrahydroisoquinoline-4-carboxylic acid (10.0 g, 37.0 mmol) and N,O-dimethylhydroxylamine HCl salt (2.94 g, 30 mmol) in DMF (300 mL) were added Et₃N (7.49 g, 74 mmol), HOBt (7.50 g, 55.5 mmol,) and EDCI (11.4 g, 59.2 mmol). The mixture was stirred at 20° C. for 16 h. The mixture was poured into brine (500 mL) and extracted with EtOAc (3×300 mL). The combined organic layers were washed with brine (400 mL), H₂O (300 mL), dried over anhydrous Na₂SO₄, filtered, and concentrated under reduced pressure. The residue was purified by silica gel column chromatography. LCMS: m/z=313.1, 315.1 [M+H]⁺.

4-acetyl-6-bromo-3,4-dihydroisoquinolin-1(2H)-one: To a solution of 6-bromo-N-methoxy-N-methyl-1-oxo-1,2,3,4-tetrahydroisoquinoline-4-carboxamide (1.0 g, 3.19 mmol) in THF (50 mL) at −20° C. was added MeMgBr (3 M in diethyl ether, 2.66 mL). Then the mixture was stirred at 20° C. for 16 h. The mixture was quenched with H₂O (10 mL) and extracted with EtOAc (3×10 mL). The combined organic layers were dried over anhydrous Na₂SO₄, filtered, and concentrated under reduced pressure. The residue was purified by silica gel column chromatography. ¹H NMR (400 MHz, CDCl₃): δ 8.00 (d, J=8.4 Hz, 1H), 7.62 (dd, J=8.4, 2.0 Hz, 1H), 7.53 (d, J=2.0 Hz, 1H), 6.04 (br s, 1H), 4.06-3.95 (m, 1H), 3.77 (dd, J=12.8, 4.4 Hz, 1H), 3.66 (dd, J=4.4, 2.4 Hz, 1H), 2.16 (s, 3H).

6-bromo-4-(1-hydroxyethyl)-3,4-dihydroisoquinolin-1(2H)-one: To a solution of 4-acetyl-6-bromo-3,4-dihydroisoquinolin-1(2H)-one (0.58 g, 2.16 mmol) in MeOH (10 mL) at 0° C. was added NaBH₄ (123 mg, 3.24 mmol). The mixture was stirred at 0° C. for 2 h. The reaction mixture was poured into cold aq. sat. NH₄Cl (20 ml). The mixture was extracted with EtOAc (3×30 mL). The combined organic layers were washed with brine (2×20 mL), dried over anhydrous Na₂SO₄, filtered, and concentrated under reduced pressure to provide a residue that was used directly.

(4r)-6-bromo-4-((1r)-1-fluoroethyl)-3,4-dihydroisoquinolin-1(2H)-one and (4r)-6-bromo-4-((is)-1-fluoroethyl)-3,4-dihydroisoquinolin-1(2H)-one: To a mixture of 6-bromo-4-(1-hydroxyethyl)-3,4-dihydroisoquinolin-1(2H)-one (0.48 g, 1.78 mmol) in DCM (40 mL) at −78° C. were added N,N-diethylethanamine;trihydrofluoride (859 mg, 5.33 mmol) and XtalFluor-E (814 mg, 3.55 mmol). The mixture was stirred at 20° C. for 16 h. The mixture was poured into H₂O (30 mL) and extracted with DCM (30 mL). The organic layer was dried over anhydrous Na₂SO₄, filtered, and concentrated under reduced pressure. The residue was purified by silica gel column chromatography to provide:

(4r)-6-bromo-4-((1r)-1-fluoroethyl)-3,4-dihydroisoquinolin-1(2H)-one: LCMS: m/z=272.0, 274.0 [M+H]⁺. ¹H NMR (400 MHz, CDCl₃): δ 7.96 (d, J=8.4 Hz, 1H), 7.58 (dd, J=8.4, 2.0 Hz, 1H), 7.44 (d, J=1.6 Hz, 1H), 6.25 (br s, 1H), 4.97-4.80 (m, 1H), 3.85-3.76 (m, 1H), 3.74-3.66 (m, 1H), 3.00-2.99 (m, 1H), 1.37-1.29 (m, 3H).

(4r)-6-bromo-4-((1s)-1-fluoroethyl)-3,4-dihydroisoquinolin-1(2H)-one: LCMS: m/z=272.0, 274.0 [M+H]⁺. ¹H NMR (400 MHz, CDCl₃): δ 7.98 (d, J=8.4 Hz, 1H), 7.58 (dd, J=8.4, 1.6 Hz, 1H), 7.48 (s, 1H), 6.12 (br s, 1H), 4.98-4.77 (m, 1H), 3.86-3.75 (m, 1H), 3.66-3.54 (m, 1H), 3.16-3.00 (m, 1H), 1.51-1.38 (m, 3H).

### ethyl 2-((4r)-6-bromo-4-((1r)-1-fluoroethyl)-1-oxo-3,4-dihydroisoquinolin-2(1H)-yl)acetate

To a solution of (4r)-6-bromo-4-((1r)-1-fluoroethyl)-3,4-dihydroisoquinolin-1(2H)-one (93 mg, 0.34 mmol) in DMF (5 mL) at 0° C. were added Cs₂CO₃ (145 mg, 0.44 mmol) and ethyl 2-iodoacetate (95 mg, 0.44 mmol). The mixture was stirred at 20° C. for 16 h. The mixture was diluted with EtOAc (10 mL) and washed with brine (3×10 mL) and H₂O (10 mL). The organic layer was dried over anhydrous Na₂SO₄, filtered, and concentrated under reduced pressure. The residue was purified by preparative thin-layer chromatography. LCMS: m/z=358.1, 360.1 [M+H]⁺.

2-[(4r)-6-bromo-4-[(1r)-1-fluoroethyl]-1-oxo-3,4-dihydroisoquinolin-2-yl]-N-(5-fluoropyrimidin-2-yl)acetamide: To a solution of ethyl 2-((4r)-6-bromo-4-((1r)-1-fluoroethyl)-1-oxo-3,4-dihydroisoquinolin-2(1H)-yl)acetate (50 mg, 0.14 mmol) and 5-fluoropyrimidin-2-amine (19 mg, 0.17 mmol) in DCE (2.0 mL) was added AlMe₃ (1 M in heptane, 0.21 mL). The mixture was stirred at 80° C. for 6 h. The mixture was quenched with H₂O (5 mL) and extracted with EtOAc (3×5 mL). The combined organic layers were dried over anhydrous Na₂SO₄, filtered, and concentrated under reduced pressure. The residue was purified by reverse-phase preparative HPLC. LCMS: m/z=424.9, 426.9 [M+H]⁺. ¹H NMR (400 MHz, CDCl₃): δ 8.88 (br s, 1H), 8.48 (s, 2H), 8.00 (d, J=8.4 Hz, 1H), 7.57 (dd, J=8.4, 1.6 Hz, 1H), 7.42 (d, J 1.2 Hz, 1H), 5.01-4.88 (m, 2H), 4.50 (d, J=16.8 Hz 1H), 4.08-4.04 (m, 1H), 3.80 (br d, J=12.0 Hz, 1H), 3.04-2.93 (m, 1H), 1.42-1.28 (m, 3H).

### Example 295 and 296

### 2-[6-bromo-4-(difluoromethyl)-5-fluoro-1-oxo-3,4-dihydroisoquinolin-2-yl]-N-(5-fluoropyrimidin-2-yl)acetamide (295 and 296)

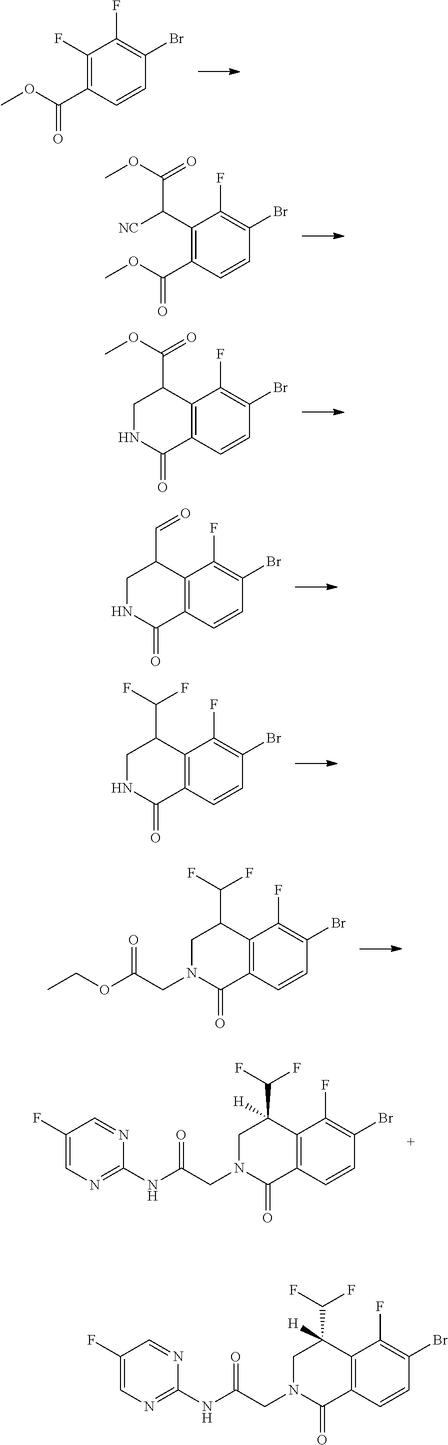

methyl 4-bromo-2-(1-cyano-2-methoxy-2-oxoethyl)-3-fluorobenzoate: To a solution of methyl 4-bromo-2,3-difluorobenzoate (4.7 g, 18.7 mmol), methyl 2-cyanoacetate (1.86 g, 18.7 mmol, 1.66 mL) in DMF (60 mL) was added Cs₂CO₃ (12.2 g, 37.5 mmol). The mixture was stirred at 90° C. for 2 h. The reaction mixture was poured into H₂O (100 mL) and aq. HCl (6 M) was added to adjust to pH=4. The mixture was extracted with EtOAc (4×20 mL). The combined organic layers were washed with brine (3×20 mL), dried over anhydrous Na₂SO₄, filtered, and concentrated under reduced pressure. The residue was triturated with MTBE:PE=1:5 (60 mL) and filtered. The filter cake was dried under reduced pressure to provide a residue that was used directly. LCMS: m/z=327.9, 329.9 [M−H]⁻.

methyl 6-bromo-5-fluoro-1-oxo-1,2,3,4-tetrahydroisoquinoline-4-carboxylate: To a solution of methyl 4-bromo-2-(1-cyano-2-methoxy-2-oxoethyl)-3-fluorobenzoate (2.5 g, 7.57 mmol) in MeOH (50 mL) and H₂O (0.5 mL) at 0° C. were added CoCl₂ (980 mg, 7.57 mmol) and NaBH₄ (860 mg, 22.7 mmol). The mixture was stirred at 0° C. for 3 h. The reaction mixture was diluted with aq. sat. NH₄Cl (50 mL) at 0° C., and extracted with DCM (4×20 mL). The combined organic layers were washed with brine (20 mL), dried over anhydrous Na₂SO₄, filtered, and concentrated under reduced pressure. The residue was purified by flash silica gel chromatography. LCMS: m/z=302.0, 304.0 [M+H]⁺.

6-bromo-5-fluoro-1-oxo-1,2,3,4-tetrahydroisoquinoline-4-carbaldehyde: To a mixture of methyl 6-bromo-5-fluoro-1-oxo-1,2,3,4-tetrahydroisoquinoline-4-carboxylate (700 mg, 2.32 mmol) in DCM (15 mL) at −78° C. was added DIBAL-H (1 M in THF, 5.80 mL). The mixture was stirred at −78° C. for 3 h. The reaction mixture was quenched by addition of aq. HCl (2M, 10 mL) at −78° C., allowed to warm to ambient temperature, diluted with H₂O (10 mL), and extracted with DCM (2×15 mL). The combined organic layers were washed with brine (30 mL), dried over anhydrous Na₂SO₄, filtered, and concentrated under reduced pressure to provide a residue that was used directly. LCMS: m/z=272.1, 274.1 [M+H]⁺.

6-bromo-4-(difluoromethyl)-5-fluoro-3,4-dihydroisoquinolin-1(2H)-one: To a solution of 6-bromo-5-fluoro-1-oxo-1,2,3,4-tetrahydroisoquinoline-4-carbaldehyde (2.65 g, 9.74 mmol) in DCM (100 mL) at −78° C. was added N,N-diethylethanamine;trihydrofluoride (4.71 g, 29.2 mmol, 4.76 mL) and (difluoro-λ⁴-sulfanylidene)-diethyl-ammonium;tetrafluoroborate (4.46 g, 19.5 mmol). The mixture was stirred at −20° C. for 3 h and then stirred at 15° C. for a further 2 h. The mixture was poured into H₂O (100 mL) and extracted with EtOAc (3×50 mL). The combined organic layers were washed with brine (20 mL), dried over anhydrous Na₂SO₄, filtered, and concentrated under reduced pressure to provide a residue that was used directly. LCMS: m/z=294.1, 296.1 [M+H]⁺.

ethyl 2-(6-bromo-4-(difluoromethyl)-5-fluoro-1-oxo-3,4-dihydroisoquinolin-2(1H)-yl)acetate: To a solution of ethyl 2-iodoacetate (1.75 g, 8.20 mmol) in DMF (30 mL) were added Cs₂CO₃ (4.01 g, 12.3 mmol) and 6-bromo-4-(difluoromethyl)-5-fluoro-3,4-dihydroisoquinolin-1(2H)-one (2.41 g, 8.20 mmol). The mixture was stirred at 25° C. for 3 h. The reaction mixture was cooled to 0° C., diluted with H₂O (100 mL), and extracted with EtOAc (4×20 mL). The combined organic layers were washed with brine (2×20 mL), dried over anhydrous Na₂SO₄, filtered, and concentrated under reduced pressure. The residue was purified by silica gel column chromatography. LCMS: m/z=380.2, 382.1 [M+H]⁺.

2-[6-bromo-4-(difluoromethyl)-5-fluoro-1-oxo-3,4-dihydroisoquinolin-2-yl]-N-(5-fluoropyrimidin-2-yl)acetamide: To a mixture of ethyl 2-(6-bromo-4-(difluoromethyl)-5-fluoro-1-oxo-3,4-dihydroisoquinolin-2(1H)-yl)acetate (200 mg, 0.53 mmol) and 5-fluoropyrimidin-2-amine (178 mg, 1.58 mmol) in DCE (7.0 mL) was added AlMe₃ (1 M in heptane, 1.58 mL). The mixture was stirred for 1.5 h at 90° C. The reaction mixture was cooled to 0° C., diluted with H₂O (10 mL), and extracted with EtOAc (4×5 mL). The combined organic layers were washed with brine (2×10 mL), dried over anhydrous Na₂SO₄, filtered, and concentrated under reduced pressure. The residue was purified by reverse-phase preparative HPLC and further purified by chiral SFC (Column: Chiralpak AD-3, (50 mm×4.6 mm, 3 μm particle size); Mobile phase: A: CO₂ and B: 0.1% i-PrNH₂ in i-PrOH; Gradient: 50% B isocratic; Flow rate: 3.4 mL/min; Detection wavelength: 220 nm; Column temperature: 35° C.; System back pressure: 124 bar) to provide:

2-[6-bromo-4-(difluoromethyl)-5-fluoro-1-oxo-3,4-dihydroisoquinolin-2-yl]-N-(5-fluoropyrimidin-2-yl)acetamide (first eluting isomer, 295): LCMS: m/z=446.9, 448.9 [M+H]⁺. ¹H NMR (400 MHz, CDCl₃): δ 9.16 (s, 1H), 8.50 (s, 2H), 7.86 (d, J=8.4 Hz, 1H), 7.68 (dd, J=6.4, 8.0 Hz, 1H), 6.22 (dt, J=5.6, 55.6 Hz, 1H), 4.95-4.64 (m, 2H), 4.15 (br d, J=12.8 Hz, 1H), 3.77 (br d, J=13.2 Hz, 1H), 3.73-3.62 (m, 1H).

2-[6-bromo-4-(difluoromethyl)-5-fluoro-1-oxo-3,4-dihydroisoquinolin-2-yl]-N-(5-fluoropyrimidin-2-yl)acetamide (second eluting isomer, 296): LCMS: m/z=446.9, 448.9 [M+H]⁺. ¹H NMR (400 MHz, CDCl₃): δ 9.13 (s, 1H), 8.50 (s, 2H), 7.86 (d, J=8.4 Hz, 1H), 7.68 (dd, J=6.4, 8.0 Hz, 1H), 6.22 (dt, J=5.6, 55.6 Hz, 1H), 4.91-4.65 (m, 2H), 4.15 (br d, J 13.2 Hz, 1H), 3.77 (br d, J=13.2 Hz, 1H), 3.73-3.62 (m, 1H).

### Example 297 and 298

### 2-[6-bromo-5-fluoro-1-oxo-4-(trifluoromethyl)-3,4-dihydroisoquinolin-2-yl]-N-(5-fluoropyrimidin-2-yl)acetamide (297 and 298)

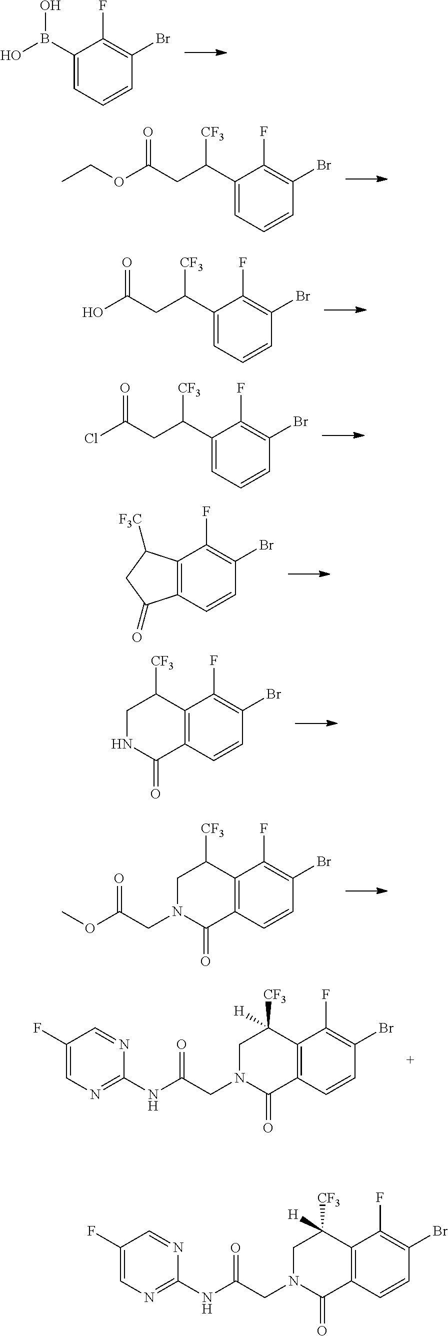

ethyl 3-(3-bromo-2-fluorophenyl)-4,4,4-trifluorobutanoate: To a mixture of (3-bromo-2-fluorophenyl)boronic acid (10.0 g, 45.7 mmol) and ethyl (E)-ethyl 4,4,4-trifluorobut-2-enoate (7.68 g, 45.7 mmol) in 1,4-dioxane (200 mL) and water (100 mL) were added chlororhodium;(1Z,5Z)-cycloocta-1,5-diene (1.13 g, 2.29 mmol) and Et₃N (13.9 g, 137 mmol). The mixture was stirred at 75° C. for 12 h. The mixture was poured into ice-cold H₂O (600 mL) and extracted with EtOAc (3×150 mL). The combined organic layers were washed with brine (200 mL), dried over anhydrous Na₂SO₄, filtered, and concentrated under reduced pressure. The residue was purified by silica gel column chromatography. LCMS: m/z=343.0, 345.0 [M+H]⁺.

3-(3-bromo-2-fluorophenyl)-4,4,4-trifluorobutanoic acid: To a mixture of ethyl 3-(3-bromo-2-fluorophenyl)-4,4,4-trifluorobutanoate (4.5 g, 13.1 mmol) in THF (50 mL) and H₂O (25 mL) was added LiOH·H₂O (1.10 g, 26.2 mmol). The mixture was stirred at 25° C. for 12 h. The mixture was poured into ice-cold H₂O (15 mL). The aqueous phase was washed with MTBE (8 mL). The aqueous phase was adjusted to pH=3 with aq. HCl (1 M) and extracted with EtOAc (3×8 mL). The combined organic layers were washed with brine (8 mL), dried over anhydrous Na₂SO₄, filtered, and concentrated under reduced pressure to provide a residue that we used directly. LCMS: m/z=312.9, 314.9 [M−H]⁻.

3-(3-bromo-2-fluorophenyl)-4,4,4-trifluorobutanoyl chloride: 3-(3-bromo-2-fluorophenyl)-4,4,4-trifluorobutanoic acid (400 mg, 1.27 mmol) was added to SOCl₂ (13.1 g, 110.3 mmol). The mixture was stirred at 80° C. for 2 h. The mixture was concentrated under reduced pressure to provide a residue that was used directly.

5-bromo-4-fluoro-3-(trifluoromethyl)-2,3-dihydro-1H-inden-1-one: To a solution of 3-(3-bromo-2-fluorophenyl)-4,4,4-trifluorobutanoyl chloride (400 mg, 1.20 mmol) in DCM (20 mL) at 0° C. was added AlCl₃ (480 mg, 3.60 mmol). The mixture was stirred at 20° C. for 16 h. The mixture was poured into ice-cold H₂O (5 mL) and extracted with DCM (3×5 mL). The combined organic layers were washed with brine (5 mL), dried over anhydrous Na₂SO₄, filtered, and concentrated under reduced pressure to provide a residue that was used directly. ¹H NMR (400 MHz, CDCl₃): δ 7.78 (dd, J=6.0, 8.0 Hz, 1H), 7.52 (d, J=8.0 Hz, 1H), 4.31 (dt, J=3.2, 8.0 Hz, 1H), 3.01-2.86 (m, 2H).

6-bromo-5-fluoro-4-(trifluoromethyl)-3,4-dihydroisoquinolin-1(2H)-one: To a mixture of 5-bromo-4-fluoro-3-(trifluoromethyl)-2,3-dihydro-1H-inden-1-one (200 mg, 0.68 mmol) and methanesulfonic acid (1.29 g, 13.5 mmol) in DCM (5 mL) at 0° C. was added NaN₃ (88 mg, 1.35 mmol). The mixture was stirred at 25° C. for 12 h. The mixture was poured into aq. sat. NaHCO₃ (20 mL) and extracted with DCM (3×5 mL). The combined organic layers were washed with brine (5 mL), dried over anhydrous Na₂SO₄, filtered, and concentrated under reduced pressure to provide a residue that was used directly. ¹H NMR (400 MHz, CDCl₃): δ 7.86 (d, J=8.4 Hz, 1H), 7.75 (dd, J=6.4, 8.4 Hz, 1H), 6.39 (br s, 1H), 3.99-3.80 (m, 3H).

methyl 2-(6-bromo-5-fluoro-1-oxo-4-(trifluoromethyl)-3,4-dihydroisoquinolin-2(1H)-yl)acetate: To a solution of 6-bromo-5-fluoro-4-(trifluoromethyl)-3,4-dihydroisoquinolin-1(2H)-one (90 mg, 0.29 mmol) and methyl 2-bromoacetate (66 mg, 0.43 mmol) in DMF (1.0 mL) were added Cs₂CO₃ (188 mg, 0.58 mmol) and NaI (43 mg, 0.29 mmol). The mixture was stirred for 2 h. The mixture was poured into H₂O (10 mL) and extracted with EtOAc (3×5 mL). The combined organic layers were washed with brine (5 mL), dried over anhydrous Na₂SO₄, filtered, and concentrated under reduced pressure. The residue was purified by silica gel column chromatography. ¹H NMR (400 MHz, CDCl₃): δ 7.88 (d, J=9.2 Hz, 1H), 7.74 (dd, J=6.4, 8.4 Hz, 1H), 4.88 (d, J=17.5 Hz, 1H), 4.28-4.19 (m, 1H), 3.95 (br d, J=4.0 Hz, 1H), 3.87-3.73 (m, 5H).

2-[6-bromo-5-fluoro-1-oxo-4-(trifluoromethyl)-3,4-dihydroisoquinolin-2-yl]-N-(5-fluoropyrimidin-2-yl)acetamide: To a solution of methyl 2-(6-bromo-5-fluoro-1-oxo-4-(trifluoromethyl)-3,4-dihydroisoquinolin-2(1H)-yl)acetate (70 mg, 0.18 mmol) and 5-fluoropyrimidin-2-amine (62 mg, 0.55 mmol) in DCE (1.0 mL) was added AlMe₃ (1 M in heptane, 0.55 mL). The mixture was stirred at 60° C. for 2 h. The reaction mixture was diluted with H₂O (5 mL) and extracted with EtOAc (3×5 mL). The combined organic layers were washed with brine (5 mL), dried over anhydrous Na₂SO₄, filtered, and concentrated under reduced pressure. The residue was purified by reverse-phase preparative HPLC and further purified by chiral SFC (Column: Chiralpak AD-3, (50 mm×4.6 mm I.D., 3 μm particle size); Mobile phase: A: CO₂ and B: 0.1% i-PrNH₂ in EtOH; Gradient: B %=40% isocratic; Detection wavelength: 220 nm; Flow rate: 4 mL/min; Column temperature: 35° C.; System back pressure: 124 bar) to provide:

2-[6-bromo-5-fluoro-1-oxo-4-(trifluoromethyl)-3,4-dihydroisoquinolin-2-yl]-N-(5-fluoropyrimidin-2-yl)acetamide (first eluting isomer, 297): LCMS: m/z=465.0, 467.0 [M+H]⁺. ¹H NMR (400 MHz, CDCl₃): δ 8.69 (br s, 1H), 8.49 (s, 2H), 7.90 (d, J=8.4 Hz, 1H), 7.74 (dd, J=6.4, 8.4 Hz, 1H), 5.39-5.13 (m, 1H), 4.39-4.21 (m, 2H), 4.05-3.91 (m, 1H), 3.84 (d, J=14.0 Hz, 1H).

2-[6-bromo-5-fluoro-1-oxo-4-(trifluoromethyl)-3,4-dihydroisoquinolin-2-yl]-N-(5-fluoropyrimidin-2-yl)acetamide (second eluting isomer, 298): LCMS: m/z=465.0, 467.0 [M+H]⁺. ¹H NMR (400 MHz, CDCl₃): δ 8.72 (br s, 1H), 8.49 (s, 2H), 7.90 (d, J=8.4 Hz, 1H), 7.74 (dd, J=6.4, 8.4 Hz, 1H), 5.37-5.13 (m, 1H), 4.37-4.21 (m, 2H), 4.03-3.90 (m, 1H), 3.84 (d, J=14.0 Hz, 1H).

### Example 299 and 300

### 2-[6-bromo-1-oxo-4-(trifluoromethyl)-3,4-dihydroisoquinolin-2-yl]-N-(5-fluoropyrimidin-2-yl)acetamide (299 and 300)

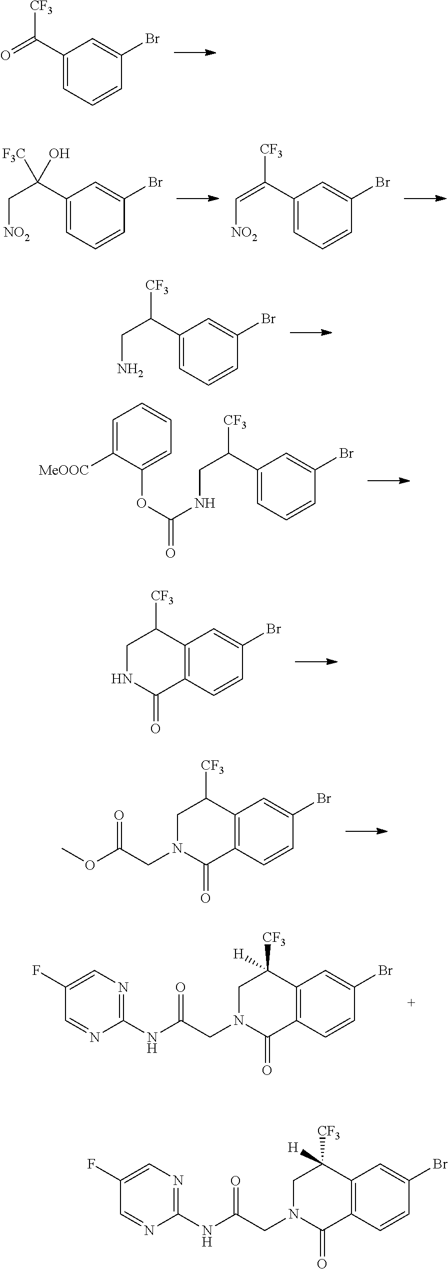

2-(3-bromophenyl)-1,1,1-trifluoro-3-nitropropan-2-ol: To a mixture of nitromethane (116 g, 1.90 mol) in THF (400 mL) at 0° C. were added 1-(3-bromophenyl)-2,2,2-trifluoro-ethanone (40 g, 158 mmol) and Et₃N (48 g, 474 mmol). The mixture was stirred at 20° C. for 12 h. The reaction mixture was diluted with H₂O (100 mL) and extracted with EtOAc (3×15 mL). The combined organic layers were washed with brine (15 mL), dried over anhydrous Na₂SO₄, filtered, and concentrated under reduced pressure. The residue was purified by silica gel column chromatography.

1-bromo-3-(3,3,3-trifluoro-1-nitroprop-1-en-2-yl)benzene: To a mixture of 2-(3-bromophenyl)-1,1,1-trifluoro-3-nitro-propan-2-ol (108 g, 343 mmol) in toluene (1000 mL) at 0° C. were added SOCl₂ (61.0 g, 516 mmol) and pyridine (54.0 g, 687 mmol). The mixture was stirred at 20° C. for 12 h. The reaction mixture was diluted with H₂O (100 mL) and extracted with EtOAc (3×25 mL). The combined organic layers were washed with brine (25 mL), dried over anhydrous Na₂SO₄, filtered, and concentrated under reduced pressure. The residue was purified by silica gel column chromatography.

2-(3-bromophenyl)-3,3,3-trifluoropropan-1-amine: To a mixture of 1-bromo-3-(3,3,3-trifluoro-1-nitroprop-1-en-2-yl)benzene (26.0 g, 88.0 mmol) in MeOH (300 mL) at 0° C. were added conc. HCl (84 mL, 878 mmol) and zinc metal (29.0 g, 439 mmol). The mixture was stirred at 0° C. for 1 h. The mixture was filtered and concentrated under reduced pressure to provide a residue that was used directly. LCMS: m/z=268.0, 270.0 [M+H]⁺.

methyl 2-(((2-(3-bromophenyl)-3,3,3-trifluoropropyl)carbamoyl)oxy)benzoate: A mixture of 2-(3-bromophenyl)-3,3,3-trifluoro-propan-1-amine (41.5 g, 155 mmol) and methyl 2-(2-methoxycarbonylphenoxy)carbonyloxybenzoate (76.7 g, 232 mmol) in THF (430 mL) was stirred for 12 h at 25° C. The mixture was concentrated under reduced pressure. The residue was purified by reverse-phase preparative HPLC.

6-bromo-4-(trifluoromethyl)-3,4-dihydroisoquinolin-1(2H)-one: To a mixture of methyl 2-[[2-(3-bromophenyl)-3,3,3-trifluoro-propyl]carbamoyloxy]benzoate (30 g, 67 mmol) in DCM (300 mL) at 0° C. was added TfOH (150 mL, 1.70 mol). The mixture was stirred at 20° C. for 12 h. The reaction mixture was diluted with H₂O (200 mL) and extracted with EtOAc (3×15 mL). The combined organic layers were washed with brine (15 mL), dried over anhydrous Na₂SO₄, filtered, and concentrated under reduced pressure. The residue was purified by silica gel column chromatography. LCMS: m/z=293.9, 295.9 [M+H]⁺.

methyl 2-(6-bromo-1-oxo-4-(trifluoromethyl)-3,4-dihydroisoquinolin-2(1H)-yl)acetate: To a mixture of 6-bromo-4-(trifluoromethyl)-3,4-dihydro-2H-isoquinolin-1-one (13.5 g, 45.9 mmol) and methyl 2-bromoacetate (7.37 g, 48.2 mmol) in DMF (150 mL) were added Cs₂CO₃ (30.0 g, 92.0 mmol) and NaI (688 mg, 4.59 mmol). The mixture was stirred for 12 h at 25° C. The reaction mixture was diluted with H₂O (100 mL) and extracted with EtOAc (3×15 mL). The combined organic layers were washed with brine (15 mL), dried over anhydrous Na₂SO₄, filtered, and concentrated under reduced pressure. The residue was purified by silica gel column chromatography. LCMS: m/z=365.9, 367.9 [M+H]⁺. ¹H NMR (400 MHz, CDCl₃): δ=8.05 (d, J=8.4 Hz, 1H), 7.66 (dd, J=2.0, 8.4 Hz, 1H), 7.54 (s, 1H), 4.80 (d, J=17.6 Hz, 1H), 3.91 (d, J=17.6 Hz, 1H), 3.85-3.69 (m, 5H), 3.61-3.58 (m, 1H).

2-[6-bromo-1-oxo-4-(trifluoromethyl)-3,4-dihydroisoquinolin-2-yl]-N-(5-fluoropyrimidin-2-yl)acetamide: To a mixture of methyl 2-[6-bromo-1-oxo-4-(trifluoromethyl)-3,4-dihydroisoquinolin-2-yl]acetate (1.0 g, 2.73 mmol) and 5-fluoropyrimidin-2-amine (618 mg, 5.46 mmol) in DCE (12 mL) was added AlMe₃ (1 M, 5.46 mL). The mixture was stirred at 60° C. for 12 hours. The reaction mixture was diluted with H₂O (20 mL) and extracted with EtOAc (3×10 mL). The combined organic layers were washed with brine (10 mL), dried over anhydrous Na₂SO₄, filtered, and concentrated under reduced pressure. The residue was purified by reverse-phase preparative HPLC and further purified by chiral SFC (Column: Phenomenex-Cellulose-2 (250 mm×30 mm, 10 μm particle size); Mobile phase: A: CO₂ B: EtOH; Gradient: B %: 45% isocratic; Flow rate: 75 g/min; Detection Wavelength: 220 nm; Column temperature: 40° C.; System back pressure: 100 bar) to provide:

2-[6-bromo-1-oxo-4-(trifluoromethyl)-3,4-dihydroisoquinolin-2-yl]-N-(5-fluoropyrimidin-2-yl)acetamide (first eluting isomer, 299): LCMS: m/z=447.0, 449.0 [M+H]⁺. ¹H NMR (400 MHz, CDCl₃): δ 8.80 (br s, 1H), 8.49 (s, 2H), 8.07 (d, J=8.4 Hz, 1H), 7.67 (d, J=8.4 Hz, 1H). 7.58-7.52 (m, 1H), 5.23-5.06 (m, 1H), 4.38-4.20 (m, 2H), 3.84 (br d, J=13.6 Hz, 1H), 3.65-3.56 (m, 1H).

2-[6-bromo-1-oxo-4-(trifluoromethyl)-3,4-dihydroisoquinolin-2-yl]-N-(5-fluoropyrimidin-2-yl)acetamide (second eluting isomer, 300): LCMS: m/z=447.0, 449.0 [M+H]⁺. ¹H NMR (400 MHz, CDCl₃): δ 8.92 (br s, 1H), 8.50 (s, 2H), 8.07 (d, J=8.4 Hz, 1H), 7.67 (dd, J=2.0, 8.4 Hz, 1H), 7.55 (s, 1H), 5.27-5.14 (m, 1H), 4.37-4.21 (m, 2H), 3.84 (dd, J=2.0, 13.6 Hz, 1H), 3.62-3.56 (m, 1H).

### Example 301

### N-(5-fluoropyrimidin-2-yl)-2-[6-iodo-1-oxo-4-(trifluoromethyl)-3,4-dihydroisoquinolin-2-yl]acetamide (301)

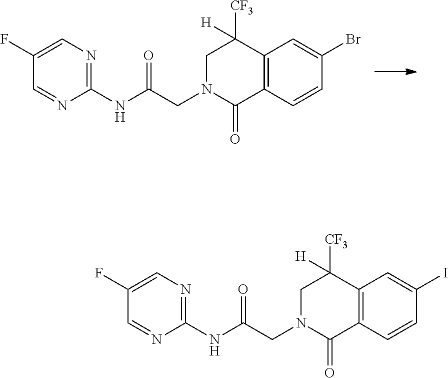 [SMILES: O=C(CN1CC(C(F)(F)F)c2cc(Br)ccc2C1=O)Nc1ncc(F)cn1 ; O=C(CN1CC(C(F)(F)F)c2cc(I)ccc2C1=O)Nc1ncc(F)cn1]

To a mixture of 2-[6-bromo-1-oxo-4-(trifluoromethyl)-3,4-dihydroisoquinolin-2-yl]-N-(5-fluoropyrimidin-2-yl)acetamide (70 mg, 0.16 mmol, Ex. 299 in 1,4-dioxane (1.0 mL) were added NaI (59 mg, 0.4 mmol), CuI (15 mg, 0.08 mmol) and (1R,2R)—N1,N2-dimethylcyclohexane-1,2-diamine (4.45 mg, 0.03 mmol). The mixture was stirred at 110° C. for 12 h. The reaction mixture was diluted with H₂O (10 mL) and extracted with EtOAc (3×5 mL). The combined organic layers were washed with brine (5 mL), dried over anhydrous Na₂SO₄, filtered, concentrated under reduced pressure, and purified by reverse-phase preparative HPLC. LCMS: m/z=494.9 [M+H]⁺. ¹H NMR (400 MHz, CDCl₃): δ 9.00 (br s, 1H), 8.50 (s, 2H), 7.93-7.87 (m, 2H), 7.76 (s, TH), 5.15 (br d, J=12.4 Hz, 1H), 4.32 (br d, J=17.2 Hz, 1H), 4.26 (dd, J=4.8, 13.2 Hz, 1H), 3.83 (dd, J=1.6, 13.6 Hz, 1H), 3.66-3.52 (m, 1H).

### Example 302

### 2-(6-ethynyl-5-fluoro-1-oxospiro[3H-isoquinoline-4,1′-cyclopropane]-2-yl)-N-(5-fluoropyrimidin-2-yl)acetamide (302)

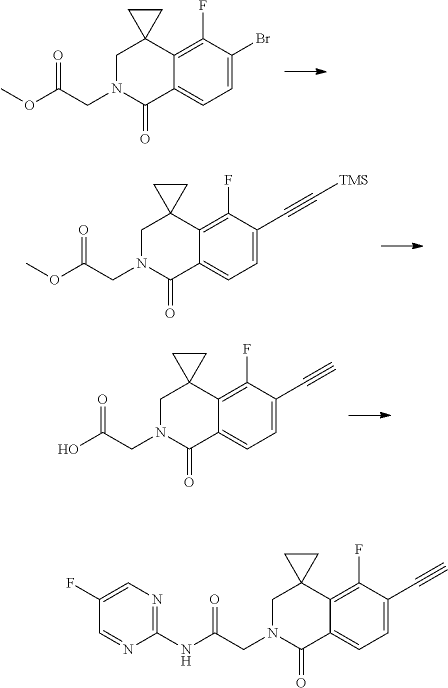

methyl 2-(5′-fluoro-1′-oxo-6′-((trimethylsilyl)ethynyl)-1′H-spiro[cyclopropane-1,4′-isoquinolin]-2′(3′H)-yl)acetate: To a solution of methyl 2-(6′-bromo-5′-fluoro-1′-oxo-1′H-spiro[cyclopropane-1,4′-isoquinolin]-2′(3′H)-yl)acetate (300 mg, 0.88 mmol, Int. 26) in THF (3.0 mL) were added ethynyltrimethylsilane (129 mg, 1.32 mmol), CuI (17 mg, 0.08 mmol), Et₃N (222 mg, 2.19 mmol), and Pd(PPh₃)₂Cl₂ (62 mg, 0.08 mmol). The reaction mixture was stirred at 50° C. for 16 h. The reaction mixture was diluted with H₂O (5 mL) and extracted with EtOAc (3×2 mL). The combined organic layers were washed with brine (5 mL), dried over anhydrous Na₂SO₄, filtered, and concentrated under reduced pressure. The residue was purified by silica gel column chromatography. LCMS: m/z=360.2 [M+H]⁺.

2-(6′-ethynyl-5′-fluoro-1′-oxo-1′H-spiro[cyclopropane-1,4′-isoquinolin]-2′(3′H)-yl)acetic acid: To a solution of methyl 2-(5′-fluoro-1′-oxo-6′-((trimethylsilyl)ethynyl)-1′H-spiro[cyclopropane-1,4′-isoquinolin]-2′(3′H)-yl)acetate (60 mg, 0.17 mmol) in THF (1.0 mL) and H₂O (1.0 mL) was added LiOH·H₂O (18 mg, 0.42 mmol). The reaction mixture was stirred for 1 h at 25° C. The reaction mixture was diluted with H₂O (5 mL) and washed with MTBE (3 mL). The aqueous phase was adjusted to pH=3 with aq. HCl (3 M) and extracted with EtOAc (3×2 mL). The combined organic layers were washed with brine (5 mL), dried over anhydrous Na₂SO₄, filtered, and concentrated under reduced to provide a residue that was used directly. LCMS: m/z=274.1 [M+H]⁺.

2-(6-ethynyl-5-fluoro-1-oxospiro[3H-isoquinoline-4,1′-cyclopropane]-2-yl)-N-(5-fluoropyrimidin-2-yl)acetamide: To a solution of 2-(6′-ethynyl-5′-fluoro-1′-oxo-1′H-spiro[cyclopropane-1,4′-isoquinolin]-2′(3′H)-yl)acetic acid (130 mg, 0.48 mmol) in pyridine (2.0 mL) were added 5-fluoropyrimidin-2-amine (135 mg, 1.19 mmol) and EDCI (182 mg, 0.95 mmol). The reaction mixture was stirred for 16 h at 25° C. The reaction mixture was diluted with H₂O (5 mL) and extracted with EtOAc (3×2 mL). The combined organic layers were washed with brine (5 mL), dried over anhydrous Na₂SO₄, filtered, and concentrated under reduced pressure. The residue was purified by reverse-phase preparative HPLC. LCMS: m/z=369.1 [M+H]⁺. ¹H NMR (400 MHz, CDCl₃): δ 9.07 (br s, 1H), 8.50 (br s, 2H), 7.95 (d, J=8.4 Hz, 1H), 7.41 (t, J=6.8 Hz, 1H). 4.62 (br s, 2H), 3.46 (s, 2H), 3.41 (s, 1H), 1.67-1.61 (m, 2H), 1.08-0.94 (m, 2H).

### Example 303 and 304

### 2-[5-fluoro-6-[(1r,2r)-2-fluorocyclopropyl]-1-oxospiro[3H-isoquinoline-4-1′-cyclopropane]-2-yl]-N-(5-fluoropyrimidin-2-yl)acetamide (303 and 304)

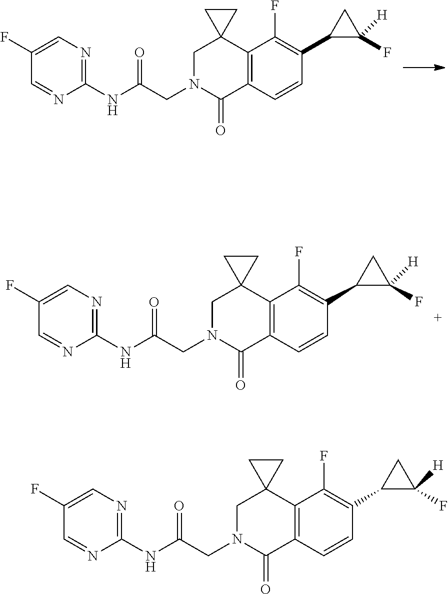

2-(5′-fluoro-6′-((1r,2r)-2-fluorocyclopropyl)-1′-oxo-1′H-spiro[cyclopropane-1,4′-isoquinolin]-2′(3′H)-yl)-N-(5-fluoropyrimidin-2-yl)acetamide (Int. 62) was further purified by chiral SFC (Column: Chiralpak AD-3 (250 mm×30 mm, 10 μm particle size); Mobile phase: A: CO₂ B: 0.1% NH₃/H₂O in MeOH; Gradient: B %: 40% isocratic; Flow rate: 4.0 mL/min; Detection Wavelength: 220 nm; Column temperature: 35° C.; System back pressure: 124 bar) to provide:

2-[5-fluoro-6-[(1r,2r)-2-fluorocyclopropyl]-1-oxospiro[3H-isoquinoline-4-1′-cyclopropane]-2-yl]-N-(5-fluoropyrimidin-2-yl)acetamide (first eluting isomer 303): LCMS: m/z=403.0 [M+H]⁺. ¹H NMR (400 MHz, CDCl₃): δ 9.03 (br s, 1H), 8.48 (s, 2H), 7.94 (d, J=8.0 Hz. 1H), 7.15 (t, J=7.2 Hz, 1H), 4.97-4.74 (m, 1H), 4.54 (br s, 2H), 3.50-3.39 (m, 2H), 2.26-2.08 (m, 1H), 1.71-1.62 (m, 2H), 1.40-1.28 (m, 2H), 1.05-0.94 (m, 2H).

2-[5-fluoro-6-[(1r,2r)-2-fluorocyclopropyl]-1-oxospiro[3H-isoquinoline-4-1′-cyclopropane]-2-yl]-N-(5-fluoropyrimidin-2-yl)acetamide (second eluting isomer 304): LCMS: m/z=403.0 [M+H]⁺. ¹H NMR (400 MHz, CDCl₃): δ 9.02 (br s, 1H), 8.48 (s, 2H), 7.94 (d, J=8.0 Hz, 1H), 7.15 (t, J=7.2 Hz, 1H), 4.99-4.75 (m, 1H), 4.58-4.47 (m, 2H), 3.50-3.40 (m, 2H), 2.25-2.11 (m, 1H), 1.72-1.63 (m, 2H), 1.39-1.29 (m, 2H), 1.04-0.94 (m, 2H).

### Example 305 and 306

### 2-[5-fluoro-6-[(1r,2s)-2-fluorocyclopropyl]-1-oxospiro[3H-isoquinoline-4,1′-cyclopropane]-2-yl]-N-(5-fluoropyrimidin-2-yl)acetamide (305 and 306)

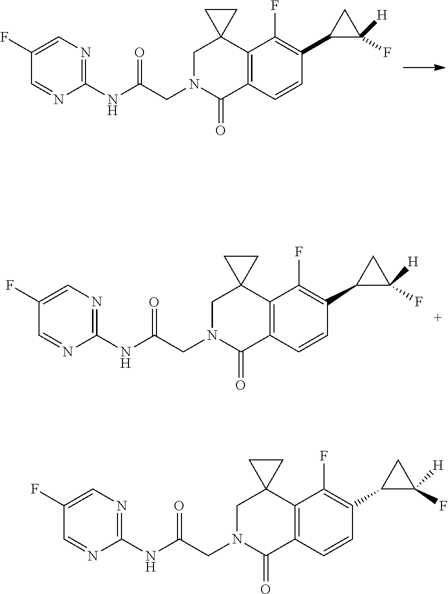

2-(5′-fluoro-6′-((1s,2r)-2-fluorocyclopropyl)-1′-oxo-1′H-spiro[cyclopropane-1,4′-isoquinolin]-2′(3′H)-yl)-N-(5-fluoropyrimidin-2-yl)acetamide (Int. 63) was further purified by chiral SFC (Column: Daicel Chiralpak IE (250 mm×30 mm, 10 μm particle size); Mobile phase: A: CO₂ B: 0.1% NH₃/H₂O in MeOH; Gradient: B %: 40% isocratic; Flow rate: 4.0 mL/min; Detection Wavelength: 220 nm; Column temperature: 35° C.; System back pressure: 124 bar) to provide:

2-[5-fluoro-6-[(1r,2s)-2-fluorocyclopropyl]-1-oxospiro[3H-isoquinoline-4,1′-cyclopropane]-2-yl]-N-(5-fluoropyrimidin-2-yl)acetamide (first eluting isomer 305): ¹H NMR (400 MHz, CDCl₃): δ 8.92 (br s, 1H), 8.48 (s, 2H), 7.90 (d, J=8.0 Hz, 1H), 6.74 (t, J=7.2 Hz, 1H), 4.79-4.59 (m, 1H), 4.55 (br s, 2H), 3.44 (s, 2H), 2.62-2.44 (m, 1H), 1.67-1.62 (m, 3H), 1.22-1.13 (m, 1H), 1.00 (s, 2H).

2-[5-fluoro-6-[(1r,2s)-2-fluorocyclopropyl]-1-oxospiro[3H-isoquinoline-4,1′-cyclopropane]-2-yl]-N-(5-fluoropyrimidin-2-yl)acetamide (second eluting isomer 306): LCMS: m/z=403.0 [M+H]⁺. ¹H NMR (400 MHz, CDCl₃): δ 8.93 (br s, 1H), 8.48 (s, 2H), 7.90 (d, J=8.0 Hz, 1H), 6.74 (t, J=7.2 Hz, 1H), 4.79-4.59 (m, 1H), 4.55 (br s, 2H), 3.44 (s, 2H), 2.61-2.46 (m, 1H), 1.67-1.59 (m, 3H), 1.22-1.12 (m, 1H), 1.00 (s, 2H).

### Example 307

### 2-[5-fluoro-6-(1-fluorocyclopropyl)-1-oxospiro[3H-isoquinoline-4,1′-cyclopropane]-2-yl]-N-(5-fluoropyrimidin-2-yl)acetamide (307)

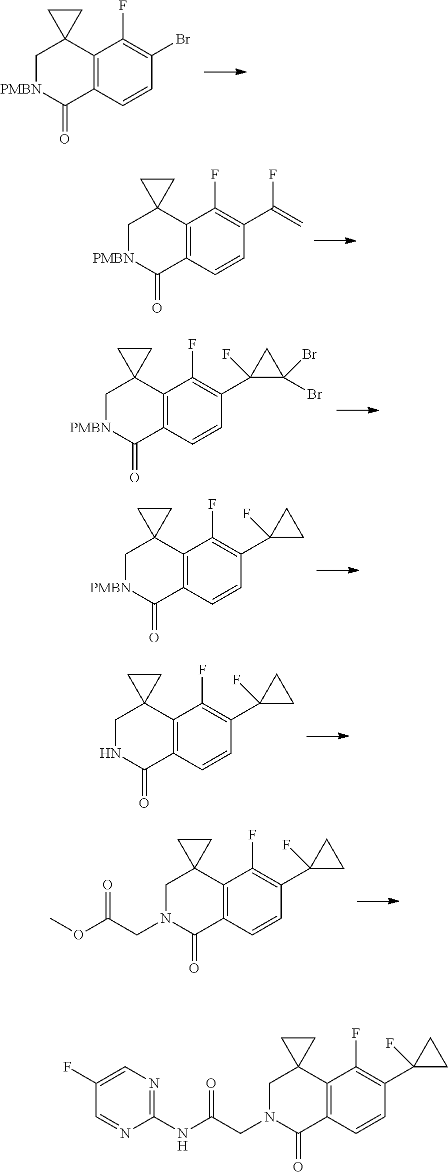

5′-fluoro-6′-(1-fluorovinyl)-2′-(4-methoxybenzyl)-2′,3′-dihydro-1′H-spiro[cyclopropane-1,4′-isoquinolin]-1′-one: To a solution of 6′-bromo-5′-fluoro-2′-(4-methoxybenzyl)-2′,3′-dihydro-1′H-spiro[cyclopropane-1,4′-isoquinolin]-1′-one (500 mg, 1.28 mmol, Int. 61) and 1-fluorovinyl-methyl-diphenyl-silane (466 mg, 1.92 mmol) in 1,3-dimethylimidazolidin-2-one (5 mL) were added CuI (49 mg, 0.26 mmol), Pd(dppf)Cl₂ (94 mg, 0.13 mmol) and CsF (487 mg, 3.20 mmol). The mixture was stirred at 20° C. for 16 h. The reaction mixture was poured into H₂O (20 mL) and extracted with EtOAc (3×10 mL). The combined organic layers were dried over anhydrous Na₂SO₄, filtered, and concentrated under reduced pressure. The residue was purified by silica gel column chromatography. LCMS: m/z=356.0 [M+H]⁺.

6′-(2,2-dibromo-1-fluorocyclopropyl)-5′-fluoro-2′-(4-methoxybenzyl)-2′,3′-dihydro-1′H-spiro[cyclopropane-1,4′-isoquinolin]-1′-one: To a solution of 5′-fluoro-6′-(1-fluorovinyl)-2′-(4-methoxybenzyl)-2′,3′-dihydro-1′H-spiro[cyclopropane-1,4′-isoquinolin]-1′-one (200 mg, 0.56 mmol) in CHBr₃ (2 mL) at 0° C. were added benzyltriethylammonium chloride (5.0 mg, 0.02 mmol) and aq. NaOH (45 mg, 0.56 mmol, 50% (w/w)). The mixture was stirred at 0° C. for 0.5 h then stirred at 20° C. for 12 h. The reaction mixture was poured into ice-cold H₂O (20 mL) and extracted with DCM (2×10 mL). The combined organic layers were washed with aq. HCl (1 M, 10 mL), aq. NaHCO₃ (5% (w/w), 10 mL), and H₂O (10 mL), dried over anhydrous Na₂SO₄, filtered, and concentrated under reduced pressure to provide a residue that was used directly. LCMS: m/z=525.8, 527.8, 529.8 [M+H]⁺.

5′-fluoro-6′-(1-fluorocyclopropyl)-2′-(4-methoxybenzyl)-2′,3′-dihydro-1′H-spiro[cyclopropane-1,4′-isoquinolin]-1′-one: To a solution of 6′-(2,2-dibromo-1-fluorocyclopropyl)-5′-fluoro-2′-(4-methoxybenzyl)-2′,3′-dihydro-1′H-spiro[cyclopropane-1,4′-isoquinolin]-1′-one (450 mg, 0.85 mmol) in tributylstannane (3.73 g, 12.8 mmol) was added AIBN (14 mg, 0.08 mmol). The mixture was stirred for 12 h at 20° C., and then stirred at 80° C. for 2 h. The reaction mixture was cooled to 20° C., poured into aq. sat. KF (10 mL), and stirred for a further 1 h at 20° C. The mixture was extracted with EtOAc (4×10 mL). The combined organic layers were washed with brine (10 mL), dried over anhydrous Na₂SO₄, filtered, and concentrated under reduced pressure to give a residue. The residue was purified by silica gel column chromatography. LCMS: m/z=370.1 [M+H]⁺.

5′-fluoro-6′-(1-fluorocyclopropyl)-2′-(4-methoxybenzyl)-2′,3′-dihydro-1′H-spiro[cyclopropane-1,4′-isoquinolin]-1′-one: 5-fluoro-6-(1-fluorocyclopropyl)-2-[(4-methoxyphenyl)methyl]spiro[3H-isoquinoline-4,1′-cyclopropane]-1-one (80 mg, 0.22 mmol) was added to TFA (1.0 mL) and the mixture was stirred at 60° C. for 6 h. The reaction mixture was poured into H₂O (5 mL) and the aqueous layer was adjusted to pH=8 with aq. sat. NaHCO₃. The mixture was extracted with EtOAc (3×5 mL). The combined organic layers were washed with brine (5 mL), dried over anhydrous Na₂SO₄, filtered and concentrated under reduced pressure to provide a residue that was used directly. LCMS: m/z=250.0 [M+H]⁺.

methyl 2-(5′-fluoro-6′-(1-fluorocyclopropyl)-1′-oxo-1′H-spiro[cyclopropane-1,4′-isoquinolin]-2′(3′H)-yl)acetate: To a mixture of 5′-fluoro-6′-(1-fluorocyclopropyl)-2′,3′-dihydro-1′H-spiro[cyclopropane-1,4′-isoquinolin]-1′-one (50 mg, 0.20 mmol) and methyl 2-bromoacetate (46 mg, 0.30 mmol) in DMF (2.0 mL) were added Cs₂CO₃ (131 mg, 0.40 mmol), NaI (30 mg, 0.20 mmol). The mixture was stirred for 16 h. The reaction mixture was diluted with H₂O (10 mL) and extracted with EtOAc (3×5 mL). The combined organic layers were washed with brine (5 mL), dried over anhydrous Na₂SO₄, filtered, and concentrated under reduced pressure. The residue was purified by preparative silica gel thin-layer chromatography. LCMS: m/z=322.1 [M+H]⁺.

2-[5-fluoro-6-(1-fluorocyclopropyl)-1-oxospiro[3H-isoquinoline-4,1′-cyclopropane]-2-yl]-N-(5-fluoropyrimidin-2-yl)acetamide: To a mixture of methyl 2-(5′-fluoro-6′-(1-fluorocyclopropyl)-1′-oxo-1′H-spiro[cyclopropane-1,4′-isoquinolin]-2′(3′H)-yl)acetate (50 mg, 0.16 mmol) and 5-fluoropyrimidin-2-amine (35 mg, 0.31 mmol) in DCE (1.0 mL) was added AlMe₃ (1 M in heptane, 0.31 mL). The mixture was stirred at 60° C. for 3 h. The reaction mixture was diluted with H₂O (10 mL) and extracted with EtOAc (3×5 mL). The combined organic layers were washed with brine (5 mL), dried over anhydrous Na₂SO₄, filtered and concentrated under reduced pressure. The residue was purified by reverse-phase preparative HPLC. LCMS: m/z=403.0 [M+H]⁺. ¹H NMR (400 MHz, CDCl₃): δ=8.92 (br s, 1H), 8.48 (s, 2H), 8.00 (d, J=8.0 Hz, 1H), 7.42 (t, J=7.2 Hz, 1H), 4.58 (br s, 2H), 3.46 (s, 2H), 1.66-1.63 (m, 2H), 1.44 (td, J=7.2, 18.8 Hz, 2H), 1.18-1.08 (m, 2H), 1.01 (s, 2H).

### Example 308

### 2-[(2′R,4S)-6-bromo-2′-fluoro-1-oxospiro[3H-isoquinoline-4,1′-cyclopropane]-2-yl]-N-[5-(trifluoromethyl)pyrimidin-2-yl]acetamide (308)

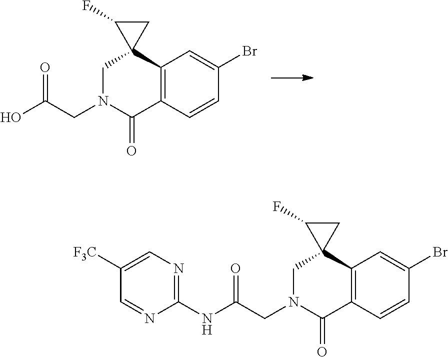

To a solution of 5-(trifluoromethyl)pyrimidin-2-amine (52 mg, 0.32 mmol) in THF (2.0 mL) at 0° C. was added LiHMDS (1 M in THF, 0.32 mL). The mixture was stirred at 0° C. for 2 h. To a solution of 2-[(2′s,4r)-6-bromo-2′-fluoro-1-oxospiro[3H-isoquinoline-4,1′-cyclopropane]-2-yl]acetic acid (70 mg, 0.23 mmol, Int. 34) in THF (1.0 mL) at 20° C. was added CDI (69 mg, 0.43 mmol). The latter mixture was stirred at 20° C. for 2 h and added to the former mixture at 0° C. The reaction mixture was stirred at 20° C. for 2 h. The mixture was poured into ice-cold H₂O (10 mL) and extracted with EtOAc (3×10 mL). The combined organic layers were concentrated under reduced pressure. The residue was purified by reverse-phase preparative HPLC. LCMS: m/z=472.9, 475.0 [M+H]⁺. ¹H NMR (400 MHz, CDCl₃): δ 9.27 (br s, 1H), 8.85 (s, 2H), 8.05 (d, J=8.4 Hz, 1H), 7.52 (dd, J=1.6, 8.4 Hz, 1H), 6.85 (d, J=1.6 Hz. 1H), 5.08 (br d, J=16.8 Hz, 1H), 4.74-4.52 (m, 1H), 4.46 (d, J=17.2 Hz, 1H), 4.19 (dd, J=1.6, 12.8 Hz, 1H), 3.52 (d, J=12.8 Hz, 1H), 1.67-1.60 (m, 1H). 1.50-1.35 (m, 1H).

### Example 309

### 2-(6-bromo-1-oxospiro[3H-isoquinoline-4,1′-cyclopropane]-2-yl)-N-[5-(2,2-difluorocyclopropyl)pyrimidin-2-yl]acetamide (309)

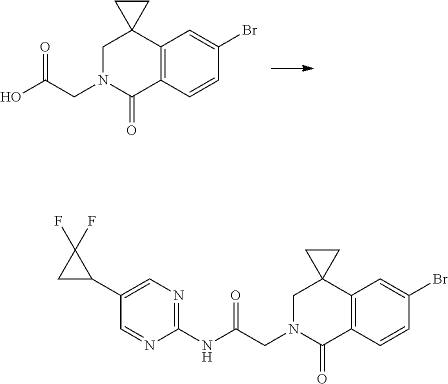 [SMILES: O=C(O)CN1CC2(CC2)c2cc(Br)ccc2C1=O ; O=C(CN1CC2(CC2)c2cc(Br)ccc2C1=O)Nc1ncc(C2CC2(F)F)cn1]

To a solution of 5-(2,2-difluorocyclopropyl)pyrimidin-2-amine (40 mg, 0.23 mmol, Int. 62) and methyl 2-(6′-bromo-1′-oxo-1′H-spiro[cyclopropane-1,4′-isoquinolin]-2′(3′H)-yl)acetate (53 mg, 0.16 mmol, Int. 3) in DCE (1.0 mL) at 0° C. was added AlMe₃ (1 M in n-hexane, 0.47 mL). The mixture was heated to 90° C., and stirred for 2 h. The mixture was diluted with H₂O (15 mL) and extracted with EtOAc (3×5 mL). The mixture was washed with brine (5 mL), dried over anhydrous Na₂SO₄, filtered, and concentrated under reduced pressure. The residue was purified by reverse-phase preparative HPLC. LCMS: m/z=463.0, 464.9 [M+H]⁺. ¹H NMR (400 MHz, CDCl₃): δ 8.84 (br s, 1H), 8.48 (s, 2H), 8.02 (d, J=8.4 Hz, 1H), 7.46 (d, J=8.4 Hz, 1H), 7.01 (s, 1H), 4.64 (s, 2H), 3.53 (s, 2H), 2.77-2.54 (m, 1H), 2.04-1.87 (m, 1H), 1.68-1.60 (m, 1H), 1.17-1.11 (m, 2H), 1.10-1.04 (m, 2H).

### Example 310

### 2-[6-(difluoromethoxy)-5-fluoro-1-oxospiro[3H-isoquinoline-4,1′-cyclopropane]-2-yl]-N-(5-fluoropyrimidin-2-yl)acetamide (310)

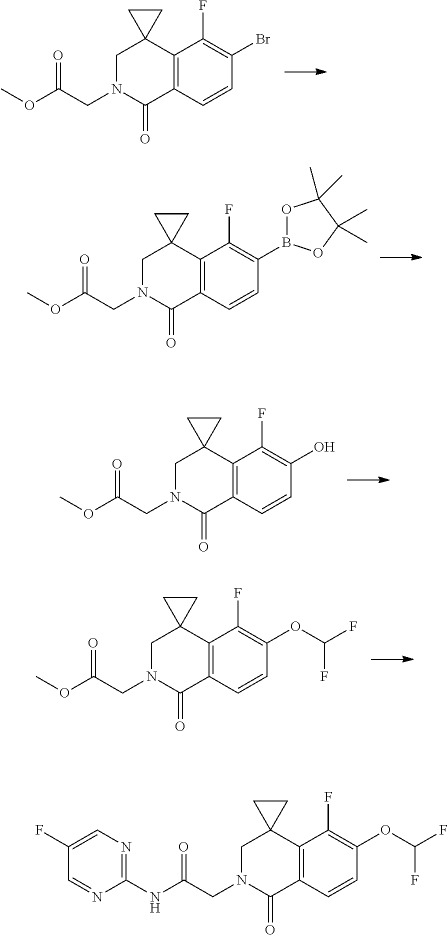

methyl 2-(5′-fluoro-1′-oxo-6′-(4,4,5,5-tetramethyl-1,3,2-dioxaborolan-2-yl)-1′H-spiro[cyclopropane-1,4′-isoquinolin]-2′(3′H)-yl)acetate: To a solution of methyl 2-(6′-bromo-5′-fluoro-1′-oxo-1′H-spiro[cyclopropane-1,4′-isoquinolin]-2′(3′H)-yl)acetate (800 mg, 2.34 mmol, Int. 26) and bis(pinacolato)diboron (890 mg, 3.50 mmol) in 1,4-dioxane (10 mL) at 20° C. were added KOAc (690 mg, 7.02 mmol) and Pd(dppf)Cl₂ (171 mg, 0.23 mmol). The mixture was stirred at 80° C. for 12 h. The mixture was diluted with H₂O (15 mL) and extracted with EtOAc (3×5 mL). The combined organic layers were washed with brine (10 mL), dried over anhydrous Na₂SO₄, filtered, and concentrated under reduced pressure. The residue was purified by silica gel column chromatography. LCMS: m/z=390.2 [M+H]⁺.

methyl 2-(5′-fluoro-6′-hydroxy-1′-oxo-1′H-spiro[cyclopropane-1,4′-isoquinolin]-2′(3′H)-yl)acetate: To a solution methyl 2-(5′-fluoro-1′-oxo-6′-(4,4,5,5-tetramethyl-1,3,2-dioxaborolan-2-yl)-1′H-spiro[cyclopropane-1,4′-isoquinolin]-2′(3′H)-yl)acetate (900 mg, 2.31 mmol) in 1,4-dioxane (9 mL) and H₂O (9 mL) at 0° C. was added Oxone (1.56 g, 2.54 mmol). The mixture was stirred at 20° C. for 4 h. The mixture was diluted with H₂O (25 mL) and extracted with EtOAc (3×10 mL). The combined organic layers were washed with brine (10 mL), dried over anhydrous Na₂SO₄, filtered, and concentrated under reduced pressure. The residue was purified by silica gel column chromatography. LCMS: m/z=280.1 [M+H]⁺.

methyl 2-(6′-(difluoromethoxy)-5′-fluoro-1′-oxo-1′H-spiro[cyclopropane-1,4′-isoquinolin]-2′(3′H)-yl)acetate: To a solution of methyl 2-(5′-fluoro-6′-hydroxy-1′-oxo-1′H-spiro[cyclopropane-1,4′-isoquinolin]-2′(3′H)-yl)acetate (300 mg, 1.07 mmol) in DMF (5.0 mL) at 20° C. were added sodium 2-chloro-2,2-difluoroacetate (377 mg, 2.47 mmol) and K₂CO₃ (297 mg, 2.15 mmol). The mixture was stirred at 110° C. for 16 h. The mixture was diluted with water (10 mL) and extracted with EtOAc (3×3 mL). The combined organic layers were washed with brine (5 mL), dried over anhydrous Na₂SO₄, filtered and concentrated under reduced pressure. The residue was purified by silica gel column chromatography. LCMS: m/z=330.2 [M+H]⁺.

2-(6′-(difluoromethoxy)-5′-fluoro-1′-oxo-1′H-spiro[cyclopropane-1,4′-isoquinolin]-2′(3′H)-yl)-N-(5-fluoropyrimidin-2-yl)acetamide: To a solution of methyl 2-(6′-(difluoromethoxy)-5′-fluoro-1′-oxo-1′H-spiro[cyclopropane-1,4′-isoquinolin]-2′(3′H)-yl)acetate (80 mg, 0.24 mmol) in DCE (1.0 mL) at 20° C. were added 5-fluoropyrimidin-2-amine (69 mg, 0.61 mmol) and AlMe₃ (1 M in heptane, 0.24 mL). The mixture was stirred at 60° C. for 12 h. The mixture was diluted with H₂O (5 mL) and extracted with EtOAc (3×2 mL). The combined organic layers were washed with brine (3 mL), dried over anhydrous Na₂SO₄, filtered, and concentrated under reduced pressure. The residue was purified by reverse-phase preparative HPLC. LCMS: m/z=411.0 [M+H]⁺. ¹H NMR (400 MHz, CDCl₃): δ 8.95 (br s, 1H), 8.49 (s, 2H), 8.00 (dd, J=8.8, 1.6 Hz, 1H), 7.17 (t, J=7.6 Hz, 1H), 6.57 (t, J=72.8, 1H), 4.60 (br s, 2H), 3.48 (s, 2H), 1.62-1.67 (m, 2H), 1.01-1.09 (m, 2H).

### Example 311

### 2-(2-cyclopropyl-5-oxospiro[7H-1,6-naphthyridine-8,1′-cyclopropane]-6-yl)-N-(5-fluoropyrimidin-2-yl)acetamide (311)

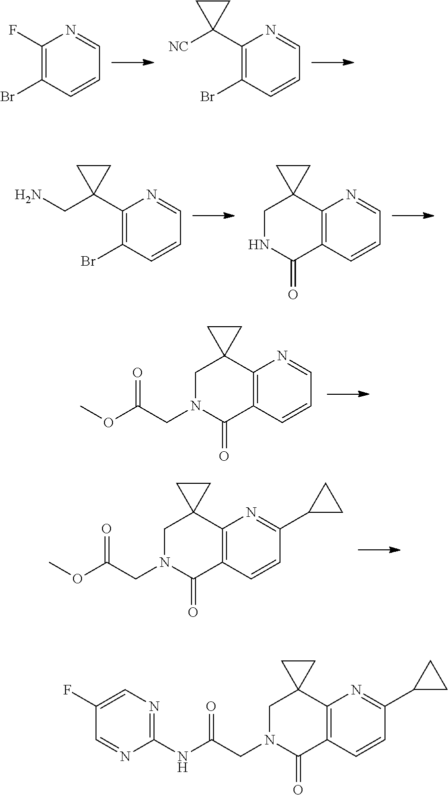

1-(3-bromopyridin-2-yl)cyclopropanecarbonitrile: To a solution of cyclopropanecarbonitrile (9.1 g, 136 mmol) and 3-bromo-2-fluoro-pyridine (20 g, 114 mmol) in toluene at −60° C. (150 mL) was added KHMDS (1 M in toluene. 125 mL, 125 mmol). The mixture was stirred at −60° C. for 4 h. The reaction mixture was poured into aq. sat. NH₄Cl (500 mL) and extracted with EtOAc (3×150 mL). The combined organic layers were washed with brine (150 mL), dried over anhydrous Na₂SO₄, filtered, and concentrated under reduced pressure. The residue was purified by silica gel column chromatography. LCMS: m/z=223.0, 225.0 [M+H]⁺.

(1-(3-bromopyridin-2-yl)cyclopropyl)methanamine: To a solution of 1-(3-bromopyridin-2-yl)cyclopropanecarbonitrile (5.28 g, 23.7 mmol) in THF (100 mL) at 0° C. was added BH₃·THF (1 M in THF, 71 mL). The mixture was stirred at 20° C. for 16 h. The mixture was quenched with MeOH (100 mL) and concentrated under reduced pressure. The residue was purified by reverse-phase preparative HPLC. LCMS: m/z=227.1, 229.1 [M+H]⁺.

6′,7′-dihydro-5′H-spiro[cyclopropane-1,8′-[1,6]naphthyridin]-5′-one: To a solution of (1-(3-bromopyridin-2-yl)cyclopropyl)methanamine (500 mg, 2.20 mmol) in toluene (100 mL) were added Pd(dba)₂ (63 mg, 0.11 mmol). Na₂CO₃ (700 mg, 6.61 mmol), and bis(1-adamantyl)-butyl-phosphane (79 mg, 0.22 mmol). The suspension was degassed under vacuum and purged with CO three times. The mixture was stirred under CO (30 Psi) at 80° C. for 16 h. The mixture was concentrated under reduced pressure. The residue was purified by silica gel column chromatography. LCMS: m/z=175.2 [M+H]⁺.

methyl 2-(5′-oxo-5′H-spiro[cyclopropane-1,8′-[1,6]naphthyridin]-6′(7′H)-yl)acetate:

To a solution of 6′,7′-dihydro-5′H-spiro[cyclopropane-1,8′-[1,6]naphthyridin]-5′-one (186 mg, 1.07 mmol) in DMF (5.0 mL) were added Cs₂CO₃ (696 mg, 2.14 mmol) and methyl 2-bromoacetate (163 mg, 1.07 mmol). The mixture was stirred at 40° C. for 6 h. The mixture was diluted with EtOAc (10 mL) and washed with brine (3×5 mL) and H₂O (5 mL). The organic layer was dried over anhydrous Na₂SO₄, filtered, and concentrated under reduced pressure. The residue was purified by silica gel column chromatography. LCMS: m/z=247.1 [M+H]⁺.

methyl 2-(2′-cyclopropyl-5′-oxo-5′H-spiro[cyclopropane-1,8′-[1,6]naphthyridin]-6′(7′H)-yl)acetate: To a solution of methyl 2-(5′-oxo-5′H-spiro[cyclopropane-1,8′-[1,6]naphthyridin]-6′(7′H)-yl)acetate (158 mg, 0.64 mmol) in DCM (1.5 mL) were added cyclopropanecarboxylic acid (110 mg, 1.28 mmol), ammonium peroxydisulfate (293 mg, 1.28 mmol), AgNO₃ (105 mg, 0.62 mol), and H₂O (1.5 mL). The reaction was stirred for 16 h at 23° C. The mixture was quenched with aq. sat. NaHCO₃ (5 mL), filtered, and the filtrate was extracted with DCM (3×10 mL). The combined organic layers were washed with brine (5 mL), dried over anhydrous Na₂SO₄, filtered, and concentrated under reduced pressure. The residue was purified by silica gel preparative thin-layer chromatography. LCMS: m/z=287.0 [M+H]⁺.

2-(2-cyclopropyl-5-oxospiro[7H-1,6-naphthyridine-8,1′-cyclopropane]-6-yl)-N-(5-fluoropyrimidin-2-yl)acetamide: To a solution of 5-fluoropyrimidin-2-amine (10 mg, 0.08 mmol) and methyl 2-(2′-cyclopropyl-5′-oxo-5′H-spiro[cyclopropane-1,8′-[1,6]naphthyridin]-6′(7′H)-yl)acetate (20 mg, 0.07 mmol) in DCE (2.0 mL) was added AlMe₃ (1 M in n-heptane, 0.1 mL). The mixture was stirred at 80° C. for 6 h. The mixture was quenched with H₂O (5 mL) and extracted with EtOAc (3×5 mL). The combined organic layers were dried over anhydrous Na₂SO₄, filtered, and concentrated under reduced pressure. The residue was purified by reverse-phase preparative HPLC. LCMS: m/z=368.0 [M+H]⁺. ¹H NMR (400 MHz, CDCl₃): δ 9.12 (br s, 1H), 8.51 (s, 2H), 8.19 (d, J=8.0 Hz, 1H), 7.09 (d, J=8.0 Hz, 1H), 4.58 (br s, 2H), 3.62 (s, 2H), 2.07-1.96 (m, 1H), 1.48-1.42 (m, 2H), 1.09-1.03 (m, 2H), 1.03-0.96 (m, 2H), 0.96-0.91 (m, 2H).

### Example 312

### 2-[6-(1-fluoroethyl)-1-oxospiro[3H-isoquinoline-4,1′-cyclopropane]-2-yl]-N-(5-fluoropyrimidin-2-yl)acetamide (312)

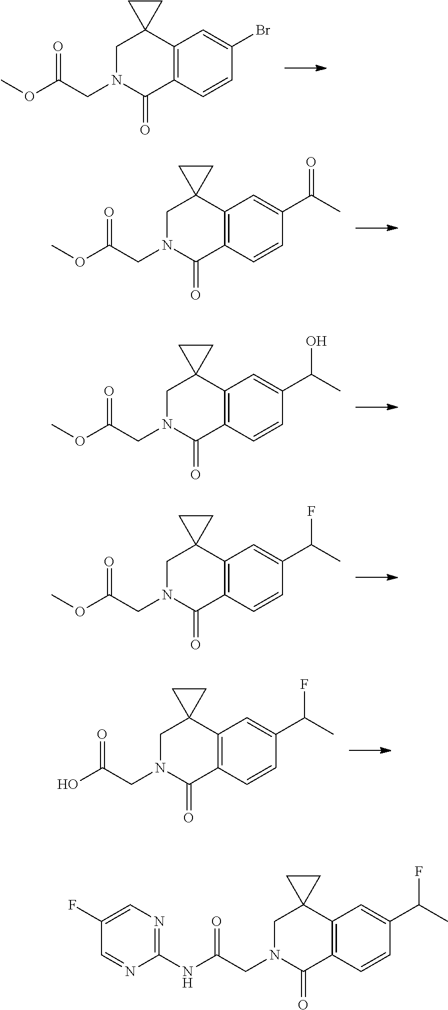

methyl 2-(6′-acetyl-1′-oxo-1′H-spiro[cyclopropane-1,4′-isoquinolin]-2′(3′H)-yl)acetate: To a solution of methyl 2-(6-bromo-1-oxo-spiro[3H-isoquinoline-4,1′-cyclopropane]-2-yl)acetate (500 mg, 1.54 mmol, Int. 2) and tributyl(1-ethoxyvinyl)stannane (670 mg, 1.85 mmol) in 1,4-dioxane (5 mL) was added Pd(PPh₃)₄ (180 mg, 0.15 mmol). The mixture was stirred at 90° C. for 12 h. The reaction mixture was cooled to 25° C., diluted with aq. HCl (2 M, 2.50 mL) and stirred for a further 1 h. The reaction mixture was concentrated under reduced pressure, resuspended in H₂O (10 mL), and the pH was adjusted (pH=8) with aq. sat. NaHCO₃. The mixture was extracted with DCM (3×10 mL) and the combined organic layers were dried over anhydrous Na₂SO₄, filtered, and concentrated under reduced pressure. The residue was purified by silica gel column chromatography. LCMS: m/z=288.0 [M+H]⁺.

methyl 2-(6′-(1-hydroxyethyl)-1′-oxo-1′H-spiro[cyclopropane-1,4′-isoquinolin]-2′(3′H)-yl)acetate: To a solution of methyl 2-(6′-acetyl-1′-oxo-1′H-spiro[cyclopropane-1,4′-isoquinolin]-2′(3′H)-yl)acetate (290 mg, 1.01 mmol) in MeOH (5 mL) at 0° C. was added NaBH₄ (95 mg, 2.52 mmol). The reaction was stirred at 0° C. for 1 h. The reaction mixture was quenched by addition of aq. sat. NH₄Cl(10 mL) and extracted with EtOAc (3×20 mL). The combined organic layers were dried over anhydrous Na₂SO₄, filtered, and concentrated under reduced pressure. The residue was purified by silica gel column chromatography. LCMS: m/z=290.1 [M+H]⁺.

methyl 2-(6′-(1-fluoroethyl)-1′-oxo-1′H-spiro[cyclopropane-1,4′-isoquinolin]-2′(3′H)-yl)acetate: To a solution of methyl 2-(6′-(1-hydroxyethyl)-1′-oxo-1′H-spiro[cyclopropane-1,4′-isoquinolin]-2′(3′H)-yl)acetate (120 mg, 0.42 mmol) in DCM (2.0 mL) at 0° C. was added DAST (133 mg, 0.83 mmol). The mixture was stirred at 0° C. for 2 h. The reaction mixture was quenched by addition of aq. sat. NaHCO₃ (10 mL) and extracted with DCM (3×10 mL). The combined organic layers were dried over anhydrous Na₂SO₄, filtered, and concentrated under reduced pressure. The residue was purified by silica gel column chromatography. LCMS: m/z=292.1 [M+H]⁺.

2-(6′-(1-fluoroethyl)-1′-oxo-1′H-spiro[cyclopropane-1,4′-isoquinolin]-2′(3′H)-yl)acetic acid: To a solution of methyl 2-(6′-(1-fluoroethyl)-1′-oxo-1′H-spiro[cyclopropane-1,4′-isoquinolin]-2′(3′H)-yl)acetate (60 mg, 0.206 mmol) in THF (1.0 mL) and H₂O (0.2 mL) was added LiOH·H₂O (17 mg, 0.41 mmol). The mixture was stirred for 1 h at 25° C. The reaction was diluted with H₂O (5 mL) and acidified to pH=3 using aq. HCl (2M). The mixture was extracted with EtOAc (3×10 mL). The combined organic layers were dried over anhydrous Na₂SO₄, filtered, and concentrated under reduced pressure to provide a residue that was used directly. LCMS: m/z=278.1 [M+H]⁺.

2-[6-(1-fluoroethyl)-1-oxospiro[3H-isoquinoline-4,1′-cyclopropane]-2-yl]-N-(5-fluoropyrimidin-2-yl)acetamide: To a mixture of 2-(6′-(1-fluoroethyl)-1′-oxo-1′H-spiro[cyclopropane-1,4′-isoquinolin]-2′(3′H)-yl)acetic acid (40 mg, 0.14 mmol) and 5-fluoropyrimidin-2-amine (24 mg, 0.22 mmol) in anhydrous pyridine (2.0 mL) at 0° C. was added EDCI (41 mg, 0.22 mmol). The mixture was stirred at 20° C. for 16 h. The mixture was concentrated under reduced pressure. The residue was purified by reverse-phase preparative HPLC. LCMS: m/z=373.1 [M+H]⁺. ¹H NMR (400 MHz, DMSO-d₆): δ 10.96 (s, 1H), 8.76 (s, 2H), 7.91 (d, J=8.0 Hz, 1H), 7.35-7.33 (m, 1H), 7.01 (s, 1H), 5.97-5.66 (m, 1H), 4.50 (s, 2H), 3.49 (s, 2H), 1.61-1.53 (m, 3H), 1.10-1.04 (m, 4H).

### Example 313

### 2-(6-ethenyl-1-oxospiro[3H-isoquinoline-4,1′-cyclopropane]-2-yl)-N-(5-fluoropyrimidin-2-yl)acetamide (313)

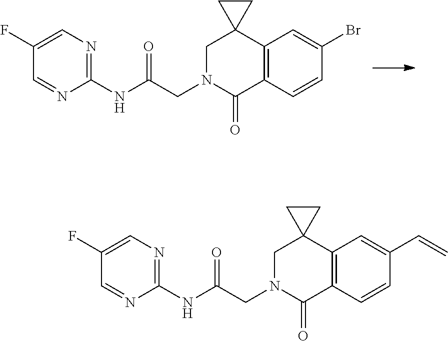 [SMILES: O=C(CN1CC2(CC2)c2cc(Br)ccc2C1=O)Nc1ncc(F)cn1 ; C=Cc1ccc2c(c1)C1(CC1)CN(CC(=O)Nc1ncc(F)cn1)C2=O]

To a solution of 2-(6′-bromo-1′-oxo-1′H-spiro[cyclopropane-1,4′-isoquinolin]-2′(3′H)-yl)-N-(5-fluoropyrimidin-2-yl)acetamide (120 mg, 0.30 mmol, Ex. 2) and potassium;trifluoro(vinyl)boranuide (80 mg, 0.60 mmol) in 1,4-dioxane (2.0 mL) were added Pd(dppf)Cl₂ (22 mg, 0.030 mmol) and CsF (135 mg, 0.89 mmol). The mixture was stirred at 90° C. for 6 h. The mixture was concentrated under reduced pressure. The residue was purified by reverse-phase preparative HPLC. LCMS: m/z=353.1 [M+H]⁺. ¹H NMR (400 MHz, MeOD): δ 8.58 (s, 2H), 7.95 (d, J=8.4 Hz, 1H), 7.42 (dd, J=1.6, 8.4 Hz, 1H), 7.03 (d, J=1.6 Hz, 1H), 6.78 (dd, J=11.2, 18.0 Hz, 1H), 5.91 (d, J=17.6 Hz, 1H), 5.36 (d, J=10.8 Hz, 1H), 4.64-4.54 (m, 2H), 3.57 (s, 2H), 1.22-1.03 (m, 4H).

### Example 314

### 2-(6-ethyl-1-oxospiro[3H-isoquinoline-4,1′-cyclopropane]-2-yl)-N-(5-fluoropyrimidin-2-yl)acetamide (314)

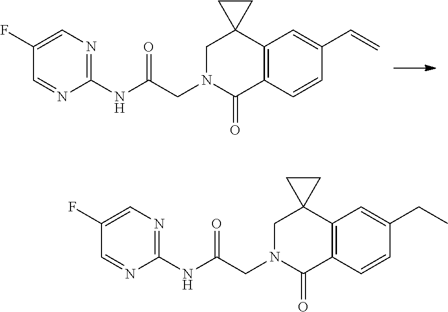 [SMILES: C=Cc1ccc2c(c1)C1(CC1)CN(CC(=O)Nc1ncc(F)cn1)C2=O ; CCc1ccc2c(c1)C1(CC1)CN(CC(=O)Nc1ncc(F)cn1)C2=O]

To a solution of N-(5-fluoropyrimidin-2-yl)-2-(1′-oxo-6′-vinyl-1′H-spiro[cyclopropane-1,4′-isoquinolin]-2′(3′H)-yl)acetamide (20 mg, 0.06 mmol, Ex. 313) in THF (1.0 mL) at 15° C. was added PtO₂ (6 mg, 0.03 mmol) and an atmosphere of H₂ was introduced. The mixture was stirred at 25° C. for 1 h under H₂. The reaction mixture was filtered and the filter cake was washed with THF (3×2 mL). The combined filtrate was concentrated under reduced pressure. The residue was purified by reverse-phase preparative HPLC. LCMS: m/z=355.1 [M+H]⁺. H NMR (400 MHz, CDCl₃): δ 9.17 (br s, 1H), 8.48 (s, 2H), 8.09 (d, J=8.0 Hz, 1H), 7.18 (d, J=8.0 Hz. 1H), 6.67 (s, 1H), 4.51 (s, 2H), 3.53 (s, 2H), 2.67 (q, J=7.6 Hz, 2H), 1.24 (t, J=7.6 Hz, 3H), 1.16-1.11 (m, 2H), 1.04-1.00 (m, 2H).

### Example 315

### N-(5-fluoropyrimidin-2-yl)-2-(6-oxospiro[8H-thieno[3,2-f]isoquinoline-9,1′-cyclopropane]-7-yl)acetamide (315)

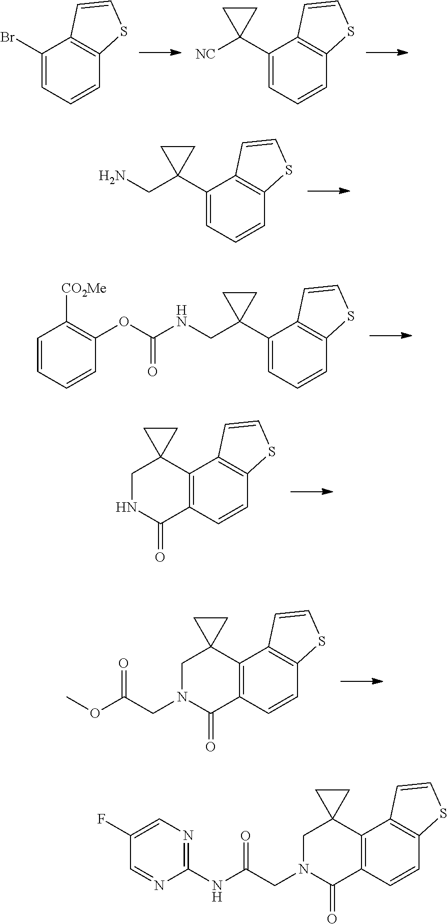

1-(benzo[b]thiophen-4-yl)cyclopropanecarbonitrile: To a degassed solution of 4-bromobenzo[b]thiophene (500 mg, 2.35 mmol), cyclopropanecarbonitrile (236 mg, 3.52 mmol, 0.26 mL), Pd₂(dba)₃ (215 mg, 0.23 mmol), BINAP (292 mg, 0.47 mmol) in THF (3 mL) was added LiHMDS (1 M in THF, 3.52 mL). The mixture was stirred at 80° C. for 3 h. The mixture was poured into aq. sat. NH₄Cl(20 mL) and extracted with EtOAc (3×10 mL). The combined organic layers were washed with brine (5 mL), dried over anhydrous Na₂SO₄, filtered, and concentrated under reduced pressure to provide a residue. The residue was purified by silica gel column chromatography.

(1-(benzo[b]thiophen-4-yl)cyclopropyl)methanamine: To a mixture of LiAlH₄ (381 mg, 10.1 mmol) in THF (4.0 mL) at 0° C. was added a solution of 1-(benzo[b]thiophen-4-yl)cyclopropanecarbonitrile (0.50 g, 2.51 mmol) in THF (2.0 mL). The mixture was stirred at 20° C. for 2 h. The mixture was quenched with H₂O (2 mL) and extracted with EtOAc (3×5 mL). The combined organic layers were washed with brine (5 mL), dried over anhydrous Na₂SO₄, filtered, and concentrated under reduced pressure to provide a residue that was used directly.

methyl 2-((((1-(benzo[b]thiophen-4-yl)cyclopropyl)methyl)carbamoyl)oxy)benzoate: To a solution of (1-(benzo[b]thiophen-4-yl)cyclopropyl)methanamine (0.45 g, 2.21 mmol) in THF (5.0 mL) was added dimethyl 2,2′-(carbonylbis(oxy))dibenzoate (730 mg, 2.21 mmol). The reaction mixture was stirred for 16 h at 20° C. The mixture was concentrated under reduced pressure to provide a residue. The residue was purified by silica gel column chromatography.

7′,8′-dihydro-6′H-spiro[cyclopropane-1,9′-thieno[3,2-f]isoquinolin]-6′-one: To a solution of methyl 2-((((1-(benzo[b]thiophen-4-yl)cyclopropyl)methyl)carbamoyl)oxy)benzoate (150 mg, 0.39 mmol) in DCM (5 mL) at 0° C. was added TfOH (472 mg, 3.15 mmol). The mixture was stirred at 0° C. for 0.5 h. The mixture was quenched with aq. sat. Na₂CO₃ (5 mL) and extracted with DCM (3×5 mL). The organic layers were dried over anhydrous Na₂SO₄, filtered, and concentrated under reduced pressure to provide a solid that was used directly.

methyl 2-(6′-oxo-6′H-spiro[cyclopropane-1,9′-thieno[3,2-f]isoquinolin]-7′(8′H)-yl)acetate: To a solution of 7′,8′-dihydro-6′H-spiro[cyclopropane-1,9′-thieno[3,2-f]isoquinolin]-6′-one (85 mg, 0.37 mmol) in DMF (2.0 mL) at 0° C. was added NaH (16 mg, 0.41 mmol, 60% purity). The mixture was stirred at 0° C. for 0.5 h. Methyl 2-bromoacetate (74 mg, 0.48 mmol) was then added and the mixture was stirred at 20° C. for a further 2 h. The mixture was quenched with H₂O (5 mL) and extracted with EtOAc (3×5 mL). The combined organic layers were washed with brine (3×5 mL), H₂O (5 mL), dried over anhydrous Na₂SO₄, filtered, and concentrated under reduced pressure. The residue was purified by silica gel column chromatography. LCMS: m/z=301.9 [M+H]⁺.

N-(5-fluoropyrimidin-2-yl)-2-(6-oxospiro[8H-thieno[3,2-f]isoquinoline-9,1′-cyclopropane]-7-yl)acetamide: To a solution of methyl 2-(6′-oxo-6′H-spiro[cyclopropane-1,9′-thieno[3,2-f]isoquinolin]-7′(8′H)-yl)acetate (46 mg, 0.15 mmol) and 5-fluoropyrimidin-2-amine (26 mg, 0.23 mmol) in toluene (3.0 mL) was added AlMe₃ (2 M in toluene, 0.23 mL). The mixture was stirred at 90° C. for 6 h. The mixture was quenched with H₂O (5 mL) and extracted with EtOAc (3×5 mL). The combined organic layers were dried over anhydrous Na₂SO₄, filtered, and concentrated under reduced pressure to provide a residue. The residue was purified by reverse-phase preparative HPLC. LCMS: m/z=383.0 [M+H]⁺. ¹H NMR (400 MHz, DMSO-d₆): δ 10.96 (br s, 1H), 8.77 (s, 2H), 7.99-7.91 (m, 2H), 7.85 (d, J=5.6 Hz, 1H), 7.57 (d, J=5.6 Hz, 1H), 4.52 (s, 2H), 3.51 (s, 2H), 1.68-1.60 (m, 2H), 1.23-1.16 (m, 2H).

### Example 316

### 2-(6-bromo-1-oxospiro[3H-isoquinoline-4,1′-cyclopropane]-2-yl)-N-[5-(methoxymethoxy)pyrimidin-2-yl]acetamide (316)

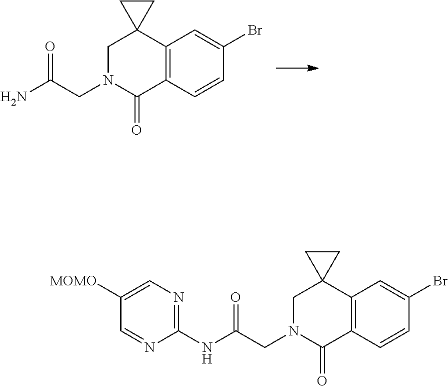

To a solution of 2-bromo-5-(methoxymethoxy)pyrimidine (141 mg, 0.64 mmol, Int. 65) in toluene (2.0 mL) were added 2-(6′-bromo-1′-oxo-1′H-spiro[cyclopropane-1,4′-isoquinolin]-2′(3′H)-yl)acetamide (200 mg, 0.65 mmol, Int. 58), t-BuONa (124 mg, 1.29 mmol), and Xantphos Pd G4 (62 mg, 0.06 mmol). The mixture was poured into H₂O (5 mL) and extracted with EtOAc (3×2 mL). The combined organic layers were washed with brine (5 mL), dried over anhydrous Na₂SO₄, filtered, and concentrated under reduced pressure. The residue was purified by reverse-phase preparative HPLC. LCMS: m/z=447.0, 449.0 [M+H]⁺. ¹H NMR (400 MHz, CDCl₃) δ 8.69 (br s, 1H), 8.41 (s, 2H), 8.02 (d, J=8.4 Hz, 1H), 7.46 (dd, J=8.0, 1.6 Hz, 1H), 7.00 (d, J=2.0 Hz. 1H), 5.18 (s, 2H), 4.60 (br s, 2H), 3.53 (s, 2H), 3.50 (s, 3H), 1.14-1.12 (m, 2H), 1.10-1.06 (m, 2H).

### Example 317 and 318

### 2-[6-bromo-1′,1′,5-trifluoro-1-oxospiro[3H-isoquinoline-4,2′-cyclopropane]-2-yl]-N-(5-chloropyrimidin-2-yl)acetamide (317 and 318)

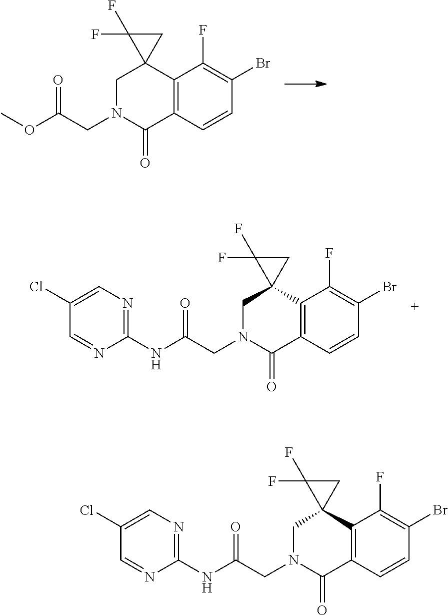 [SMILES: COC(=O)CN1CC2(CC2(F)F)c2c(ccc(Br)c2F)C1=O ; O=C(CN1C[C@@]2(CC2(F)F)c2c(ccc(Br)c2F)C1=O)Nc1ncc(Cl)cn1 ; O=C(CN1C[C@]2(CC2(F)F)c2c(ccc(Br)c2F)C1=O)Nc1ncc(Cl)cn1]

To a solution of methyl 2-(6′-bromo-2,2,5′-trifluoro-1′-oxo-1′H-spiro[cyclopropane-1,4′-isoquinolin]-2′(3′H)-yl)acetate (50 mg, 0.13 mmol, Int. 66) in DCE (2.0 mL) were added 5-chloropyrimidin-2-amine (21 mg, 0.16 mmol) and AlMe₃ (1 M in heptane, 0.2 mL). The mixture was stirred at 60° C. for 16 h. The reaction mixture was diluted with H₂O (10 mL) and extracted with EtOAc (3×8 mL). The combined organic layers were washed with brine (6 mL), dried over anhydrous Na₂SO₄, filtered, and concentrated under reduced pressure. The residue was purified by reverse-phase preparative HPLC and further purified by chiral SFC (Column: (S,S)-WHELK-O1 (50 mm×4.6 mm I.D., 3.5 μm particle size); Mobile phase: A: CO₂ B: 0.1% i-PrNH₂ in i-PrOH; Gradient: B %=50% isocratic; Detection wavelength: 220 nm; Flow rate: 3.4 mL/min; Column temperature: 35° C.; System back pressure: 124 bar) to provide:

2-[6-bromo-1′,1′,5-trifluoro-1-oxospiro[3H-isoquinoline-4,2′-cyclopropane]-2-yl]-N-(5-chloropyrimidin-2-yl)acetamide (first eluting isomer, 317): LCMS: m/z=474.9, 476.9, 478.9 [M+H]⁺. ¹H NMR (400 MHz, CDCl₃) δ 8.67 (br s, 1H), 8.55 (s, 2H), 7.92 (d, J=8.4 Hz, 1H), 7.66 (dd, J=6.4, 8.4 Hz, 1H), 5.27 (br d, J=16.8 Hz, 1H), 4.49 (dd, J=6.8, 12.8 Hz, 1H), 4.27 (d, J=17.2 Hz, 1H), 3.12 (dd, J=1.6, 13.2 Hz, 1H), 2.97-2.91 (m, 1H), 1.70-1.61 (m, 1H)

2-[6-bromo-1′,1′,5-trifluoro-1-oxospiro[3H-isoquinoline-4,2′-cyclopropane]-2-yl]-N-(5-chloropyrimidin-2-yl)acetamide (second eluting isomer, 318): LCMS: m/z=474.9, 476.9, 478.9 [M+H]⁺. ¹H NMR (400 MHz, CDCl₃) δ 8.55 (m, 3H), 7.92 (d, J=8.0 Hz, 1H), 7.67 (dd, J=6.4, 8.4 Hz, 1H), 5.26-5.22 (m, 1H), 4.48 (dd, J=6.8, 13.2 Hz, 1H), 4.26 (d, J=16.8 Hz, 1H), 3.13-3.09 (m, 1H), 2.97-2.91 (m, 1H), 1.70-1.60 (m, 1H)

### Example 319 and 320

### 2-[6-bromo-1′,1′,5-trifluoro-1-oxospiro[3H-isoquinoline-4,2′-cyclopropane]-2-yl]-N-(5-cyanopyrimidin-2-yl)acetamide (319 and 320)

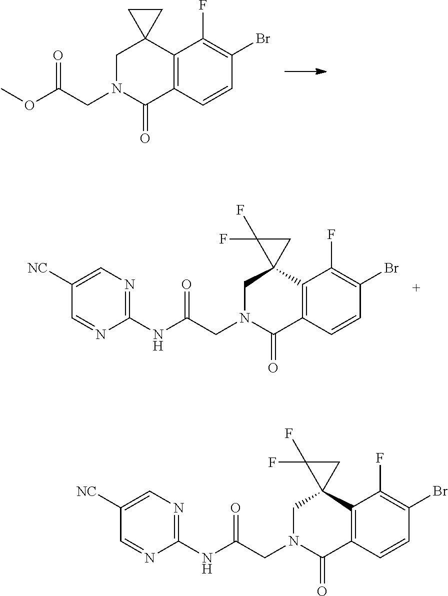 [SMILES: COC(=O)CN1CC2(CC2)c2c(ccc(Br)c2F)C1=O ; N#Cc1cnc(NC(=O)CN2C[C@@]3(CC3(F)F)c3c(ccc(Br)c3F)C2=O)nc1 ; N#Cc1cnc(NC(=O)CN2C[C@]3(CC3(F)F)c3c(ccc(Br)c3F)C2=O)nc1]

To a solution of methyl 2-(6′-bromo-2,2,5′-trifluoro-1′-oxo-1′H-spiro[cyclopropane-1,4′-isoquinolin]-2′(3′H)-yl)acetate (100 mg, 0.26 mmol, Int. 66) and 2-aminopyrimidine-5-carbonitrile (95 mg, 0.80 mmol) in DCE (5.0 mL) was added AlMe₃ (1 M in n-heptane, 0.79 mL). The mixture was stirred at 60° C. for 1 h and then stirred at 90° C. for a further 2 h. The reaction mixture was diluted with H₂O (20 mL) and extracted with EtOAc (3×10 mL). The combined organic layers were washed with brine (10 mL), dried over anhydrous Na₂SO₄, filtered, and concentrated under reduced pressure. The residue was purified by reverse-phase preparative HPLC and further purified by chiral SFC (Column: DAICEL Chiralpak AD (250 mm×30 mm, 10 μm particle size); Mobile phase: A: CO₂ B: 0.1% NH₄OH in i-PrOH; Gradient: B %=46% isocratic; Detection wavelength: 220 nm; Flow rate: 75 g/min; Column temperature: 40° C.; System back pressure: 100 bar) to provide:

2-[6-bromo-1′,1′,5-trifluoro-1-oxospiro[3H-isoquinoline-4,2′-cyclopropane]-2-yl]-N-(5-cyanopyrimidin-2-yl)acetamide (first eluting isomer, 319): LCMS: m/z=465.9, 467.9 [M+H]⁺. ¹H NMR (400 MHz, CDCl₃): δ 8.85 (s, 2H), 8.77 (br s, 1H), 7.92 (d, J=8.4 Hz, 1H), 7.66 (dd, J=6.4, 8.4 Hz, 1H), 5.23 (d, J=17.2 Hz, 1H), 4.45 (dd, J=6.8, 12.8 Hz, 1H), 4.29 (d, J=17.2 Hz, 1H), 3.11 (br d, J=13.6 Hz, 1H), 2.95 (br d, J=1.2 Hz, 1H), 1.63-1.61 (m, 1H).

2-[6-bromo-1′,1′,5-trifluoro-1-oxospiro[3H-isoquinoline-4,2′-cyclopropane]-2-yl]-N-(5-cyanopyrimidin-2-yl)acetamide (second eluting isomer, 320): LCMS: m/z=465.9, 467.9 [M+H]⁺. ¹H NMR (400 MHz, CDCl₃): δ 8.85 (s, 2H), 8.81 (br s, 1H), 7.92 (d, J=8.4 Hz, 1H), 7.66 (dd, J=6.4, 8.4 Hz, 1H), 5.29-5.16 (m, 1H), 4.45 (dd, J=6.8, 12.8 Hz, 1H), 4.29 (d, J=17.2 Hz, 1H), 3.15-3.07 (m, 1H), 3.02-2.89 (m, 1H), 1.64-1.61 (m, 1H).

### Example 321

### N-(cis-3-hydroxy-3-methylcyclobutyl)-2-[(2′R,4S)-6-bromo-2′-fluoro-1-oxospiro[3H-isoquinoline-4,1′-cyclopropane]-2-yl]acetamide (321)

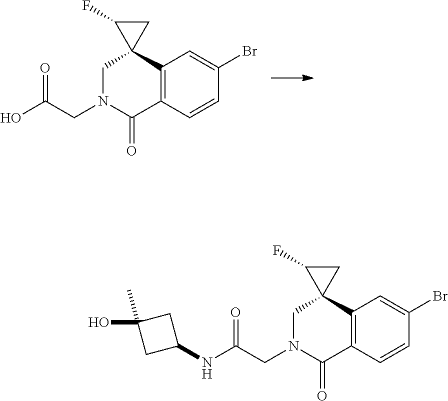

To a solution of 3-cis-amino-1-methylcyclobutanol HCl salt (29 mg, 0.21 mmol) and 2-[(2′s,4r)-6-bromo-2′-fluoro-1-oxospiro[3H-isoquinoline-4,1′-cyclopropane]-2-yl]acetic acid (70 mg, 0.10 mmol, Int. 34) in DMF (1.0 mL) at 0° C. were added HOBt (22 mg, 0.16 mmol), DIPEA (69 mg, 0.53 mmol), and EDCI (31 mg, 0.16 mmol). The mixture was stirred at 20° C. for 12 h. The reaction mixture was poured into H₂O (3 mL) and extracted with EtOAc (3×2 mL). The combined organic layers were washed with brine (5 mL), dried over anhydrous Na₂SO₄, filtered, and concentrated under reduced pressure to provide a residue. The residue was purified by reverse-phase preparative HPLC. LCMS: m/z=411.0, 413.0 [M+H]⁺. ¹H NMR (400 MHz, DMSO-d₆): δ 8.16 (d, J=7.2 Hz, 1H), 7.82 (d, J=8.4 Hz, 1H), 7.58 (dd, J=2.0, 8.4 Hz, 1H), 7.22 (d, J=2.0 Hz, 1H), 5.16-4.93 (m, 2H), 4.28 (d, J=16.4 Hz, 1H), 3.92-3.81 (m, 2H), 3.81-3.70 (m, 1H), 3.46 (d, J=13.0 Hz, 1H), 2.23-2.19 (m, 2H), 1.95-1.90 (m, 2H), 1.75-1.72 (m, 1H), 1.55-1.42 (m, 1H), 1.21 (s, 3H).

### Example 322

### N-(5-fluoropyrimidin-2-yl)-2-[6-cyclopropyl-1′,1′,5-trifluoro-1-oxospiro[3H-isoquinoline-4,2′-cyclopropane]-2-yl]acetamide (322)

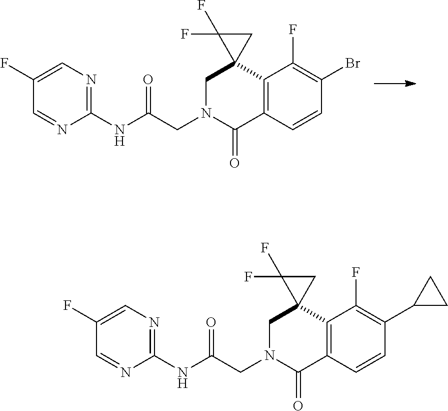

To a solution of potassium cyclopropyltrifluoroborate (24 mg, 0.16 mmol) and N-(5-fluoropyrimidin-2-yl)-2-[6-bromo-1′,1′,5-trifluoro-1-oxospiro[3H-isoquinoline-4,2′-cyclopropane]-2-yl]acetamide (15 mg, 0.03 mmol, Ex. 289) in toluene (1.0 mL) and H₂O (0.1 mL) were added bis(1-adamantyl)-butyl-phosphane (2 mg, 0.006 mmol), Pd(OAc)₂ (1 mg, 0.005 mmol), and Cs₂CO₃ (21 mg, 0.03 mmol). The reaction was stirred at 100° C. for 3 h. The reaction mixture was diluted with H₂O (2 mL) and extracted with EtOAc (3×1 mL). The combined organic layers were washed with brine (2 mL), dried over anhydrous Na₂SO₄, filtered, and concentrated under reduced pressure to provide a residue. The residue was purified by reverse-phase preparative HPLC. LCMS: m/z=421.1 [M+H]⁺. ¹H NMR (400 MHz, CDCl₃): δ 8.68 (br s, 1H), 8.48 (s, 2H), 7.93 (d, J=8.0 Hz, 1H), 6.91 (t, J=7.6 Hz, 1H), 5.15-4.96 (m, 1H), 4.47-4.33 (m, 1H), 4.22 (d, J=16.8 Hz, 1H), 3.12 (br d, J=13.2 Hz, 1H), 3.05-2.91 (m, 1H), 2.18-2.07 (m, 1H), 1.27-1.28 (m, 1H), 1.09-1.06 (m, 2H), 0.88-0.69 (m, 2H).

### Example 323 and 324

### 2-[6-bromo-1′,1′,5-trifluoro-1-oxospiro[3H-isoquinoline-4,2′-cyclopropane]-2-yl]-N-[5-(trifluoromethyl)pyrimidin-2-yl]acetamide (323 and 324)

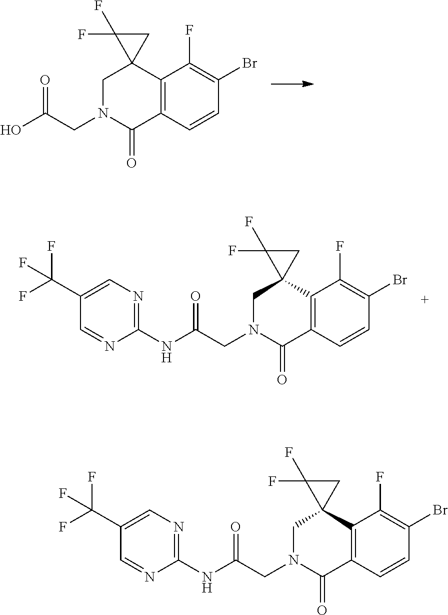 [SMILES: O=C(O)CN1CC2(CC2(F)F)c2c(ccc(Br)c2F)C1=O ; O=C(CN1C[C@@]2(CC2(F)F)c2c(ccc(Br)c2F)C1=O)Nc1ncc(C(F)(F)F)cn1 ; O=C(CN1C[C@]2(CC2(F)F)c2c(ccc(Br)c2F)C1=O)Nc1ncc(C(F)(F)F)cn1]

To a solution of 5-(trifluoromethyl)pyrimidin-2-amine (134 mg, 0.82 mmol) in THF (4 mL) at 0° C. was added LiHMDS (1 M in THF, 0.82 mL). The mixture was stirred at 0° C. for 2 h. Separately, to a solution of 2-(6′-bromo-2,2,5′-trifluoro-1′-oxo-1′H-spiro[cyclopropane-1,4′-isoquinolin]-2′(3′H)-yl)acetic acid (200 mg, 0.55 mmol, Int. 67) in THF (4.0 mL) was added CDI (178 mg, 1.10 mmol). The latter mixture was stirred at 20° C. for 2 h and added to the former mixture at 0° C. The reaction was stirred at 0° C. for 0.5 h and at 20° C. for a further 1.5 h. The reaction mixture was poured into H₂O (10 mL) and extracted with EtOAc (3×10 mL). The organic layers were concentrated under reduced pressure to provide a residue. The residue was purified by reverse-phase preparative HPLC and further purified by chiral SFC (Column: REGIS(s,s) WHELK-O1 (250 mm×30 mm×10 μm particle size); Mobile phase: A: CO₂ and B: 0.1% NH₄OH in i-PrOH; Gradient: B %: 35% isocratic; Flow rate: 70 g/min; Detection wavelength: 220 nm; Column temperature: 35° C.; System back pressure: 120 bar) to provide:

2-[6-bromo-1′,1′,5-trifluoro-1-oxospiro[3H-isoquinoline-4,2′-cyclopropane]-2-yl]-N-[5-(trifluoromethyl)pyrimidin-2-yl]acetamide (first eluting isomer, 323): LCMS: m/z=508.9, 510.9 [M+H]⁺. H NMR (400 MHz, CDCl₃): δ 8.85 (br s, 1H), 8.77 (s, 2H), 7.84 (d, J=8.4 Hz, 1H), 7.57 (dd, J=6.4, 8.4 Hz, 1H), 5.25 (br d, J=18.0 Hz, 1H), 4.39 (dd, J=6.8, 12.8 Hz, 1H), 4.23 (d, J=17.4 Hz, 1H), 3.02 (dd, J=1.8, 12.8 Hz, 1H), 2.92-2.81 (m, 1H), 1.60-1.51 (m, 1H).

2-[6-bromo-1′,1′,5-trifluoro-1-oxospiro[3H-isoquinoline-4,2′-cyclopropane]-2-yl]-N-[5-(trifluoromethyl)pyrimidin-2-yl]acetamide (second eluting isomer, 324): LCMS: m/z=508.9, 510.9 [M+H]⁺. ¹H NMR (400 MHz, CDCl₃): δ 8.89-8.82 (m, 3H), 7.92 (dd, J=0.8, 8.4 Hz, 1H), 7.65 (dd, J=6.4, 8.4 Hz, 1H), 5.33 (br d, J=17.2 Hz, 1H), 4.47 (dd, J=6.8, 12.8 Hz, 1H), 4.31 (d, J=17.2 Hz, 1H), 3.10 (dd, J=1.8, 12.8 Hz, 1H), 3.01-2.89 (m, 1H), 1.61 (dt, J=2.8, 6.0 Hz, 1H).

### Example 325 and 326

### N-(3-cis-hydroxy-3-methylcyclobutyl)-2-[6-bromo-1′,1′,5-trifluoro-1-oxospiro[3H-isoquinoline-4,2′-cyclopropane]-2-yl]acetamide (325 and 326)

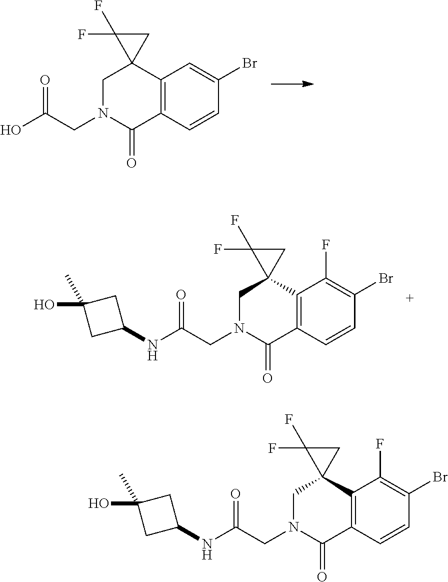

To a solution of 2-(6′-bromo-2,2,5′-trifluoro-1′-oxo-1′H-spiro[cyclopropane-1,4′-isoquinolin]-2′(3′H)-yl)acetic acid (50 mg, 0.13 mmol, Int 67) in DMF (1.0 mL) at 0° C. were added 3-cis-amino-1-methylcyclobutanol HCl salt (38 mg, 0.27 mmol), DIPEA (90 mg, 0.68 mmol), HOBt (28 mg, 0.2 mmol), and EDCI (39 mg, 0.2 mmol). The mixture was stirred at 20° C. for 12 h. The reaction mixture was diluted with H₂O (5 mL) and extracted with EtOAc (3×5 mL). The combined organic layers were washed with brine (10 mL), dried over anhydrous Na₂SO₄, filtered, and concentrated under reduced pressure to provide a residue. The residue was purified by reverse-phase preparative HPLC and further purified by chiral SFC (Column: REGIS(s,s) WHELK-O1 (250 mm×30 mm×10 μm particle size); Mobile phase: A: CO₂ and B: 0.1% NH₄OH in i-PrOH; Gradient: 30% B isocratic; Flow rate: 70 g/min; Detection wavelength: 220 nm; Column temperature: 35° C.; System back pressure: 120 bar) to provide:

N-(3-cis-hydroxy-3-methylcyclobutyl)-2-[6-bromo-1′,1′,5-trifluoro-1-oxospiro[3H-isoquinoline-4,2′-cyclopropane]-2-yl]acetamide (first eluting isomer, 325): LCMS: m/z=447.0, 449.0 [M+H]⁺. ¹H NMR (400 MHz, CDCl₃): δ 7.88 (d, J=8.4 Hz, 1H), 7.71-7.59 (m, 1H), 6.42 (br d, J=7.8 Hz, 1H), 4.27 (dd, J=6.8, 13.0 Hz, 1H), 4.16 (s, 2H), 3.98 (d, J=7.8 Hz, 1H), 3.17 (br d, J=13.0 Hz, 1H), 2.94 (br t, J=9.8 Hz, 1H), 2.52 (br dd, J=8.0, 12.0 Hz, 2H), 2.04 (br dd, J=8.4, 12.0 Hz, 2H), 1.69-1.58 (m, 1H), 1.38 (s, 3H).

N-(3-cis-hydroxy-3-methylcyclobutyl)-2-[6-bromo-1′,1′,5-trifluoro-1-oxospiro[3H-isoquinoline-4,2′-cyclopropane]-2-yl]acetamide (second eluting isomer, 326): LCMS: m/z=447.0, 449.0 [M+H]⁺. ¹H NMR (400 MHz, CDCl₃): δ 7.89 (d, J=8.6 Hz, 1H), 7.66 (dd, J=6.2, 8.4 Hz, 1H), 6.36 (br d, J=6.4 Hz, 1H), 4.26 (dd, J=6.8, 13.2 Hz, 1H), 4.16 (d, J=4.0 Hz, 2H), 4.03-3.92 (m, 1H), 3.17 (dd, J=1.8, 13.0 Hz, 1H), 3.00-2.89 (m, 1H), 2.54-2.50 (m, 2H), 2.03 (br d, J=4.0 Hz, 2H), 1.62 (br dd, J=2.6, 6.4 Hz, 1H), 1.38 (s, 3H).

### Example 327 and 328

### N-(3-cis-hydroxy-3-methylcyclobutyl)-2-[(2′s,4r)-2′-fluoro-1-oxo-6-(trifluoromethyl)spiro[3H-isoquinoline-4,1′-cyclopropane]-2-yl]acetamide (327 and 328)

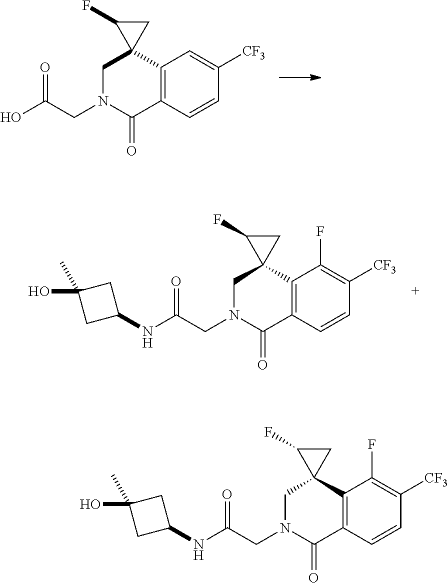

To a solution of 2-[(2′s,4r)-2′-fluoro-1-oxo-6-(trifluoromethyl)spiro[3H-isoquinoline-4,1′-cyclopropane]-2-yl]acetic acid (100 mg, 0.32 mmol, Int. 49) in DMF (2.0 mL) at 0° C. were added 3-cis-amino-1-methylcyclobutanol HCl salt (64 mg, 0.46 mmol), DIPEA (203 mg, 1.58 mmol, 0.27 mL), HOBt (64 mg, 0.47 mmol), and EDCI (90.64 mg, 0.47 mmol). The mixture was stirred at 20° C. for 12 h. The reaction mixture was poured into H₂O (10 mL) and extracted with EtOAc (4×5 mL). The combined organic layers were washed with brine (2×10 mL), dried over anhydrous Na₂SO₄, filtered, and concentrated under reduced pressure to provide a residue. The residue was purified by reverse-phase preparative HPLC and further purified by chiral SFC (Column: Chiralpak AD-3 (150 mm×4.6 mm I.D., 3 μm particle size); Mobile phase: A: CO₂ and B: 0.1% i-PrNH₂ in EtOH; Gradient: 50% B isocratic; Detection wavelength: 220 nm; Flow rate: 2.5 mL/min; Column temperature: 35° C.; System back pressure: 137 bar) to provide:

N-(3-cis-hydroxy-3-methylcyclobutyl)-2-[(2′s,4r)-2′-fluoro-1-oxo-6-(trifluoromethyl)spiro[3H-isoquinoline-4,1′-cyclopropane]-2-yl]acetamide (first eluting isomer, 327): LCMS: m/z=401.1 [M+H]⁺. ¹H NMR (400 MHz, CDCl₃): δ 8.29 (d, J=8.0 Hz, 1H), 7.66 (d, J=7.2 Hz, 1H), 6.95 (s, 1H). 6.57 (br d, J=7.2 Hz, 1H), 4.70-4.51 (m, 1H), 4.30-4.26 (m, 1H), 4.19-4.13 (m, 2H), 4.04-3.98 (m, 1H), 3.53 (d, J=12.8 Hz, 1H), 2.54-2.49 (m, 2H), 2.06-2.03 (m, 2H), 1.77-1.70 (m, 2H), 1.50-1.42 (m, 1H), 1.37 (s, 3H).

N-(3-cis-hydroxy-3-methylcyclobutyl)-2-[(2′s,4r)-2′-fluoro-1-oxo-6-(trifluoromethyl)spiro[3H-isoquinoline-4,1′-cyclopropane]-2-yl]acetamide (second eluting isomer, 328): LCMS: m/z=401.1 [M+H]⁺. ¹H NMR (400 MHz, CDCl₃): δ 8.29 (d, J=8.4 Hz, 1H), 7.66 (d, J=8.0 Hz, 1H), 6.95 (s, 1H), 6.52 (br d, J=6.4 Hz, 1H), 4.70-4.51 (m, 1H), 4.31-4.27 (m, 1H), 4.17-4.13 (m, 2H), 4.02-3.96 (m, 1H), 3.53 (d, J=13.2 Hz, 1H), 2.54-2.49 (m, 2H), 2.05 (br t, J=9.2 Hz, 2H), 1.77-1.71 (m, 2H), 1.50-1.42 (m, 1H), 1.37 (s, 3H).

The following compounds as shown in Table 1 were, or can be, made via similar procedures as those described above.

| Ex. | Name | NMR | LCMS |
|---|---|---|---|
| 329 | 2-(6-bromo-1-oxospiro[3H-isoquinoline- | ¹H NMR (400 MHz; DMSO-d₆): | m/z = |
| | 4,1′-cyclopropane]-2-yl)-N-(5,6- | δ 10.59 (s, 1H), 8.28 (t, J = 1.2 | 429.2, |
| | dihydrofuro[2,3-d]pyrimidin-2- | Hz, 1H), 7.81 (d, J = 8.3 Hz, 1H), | 431.2 |
| | yl)acetamide | 7.54-7.51 (m, 1H), 7.26 (d, J = | [M + H]⁺ |
| | | 1.9 Hz, 1H), 4.69 (t, J = 8.6 Hz, | |
| | | 2H), 4.54-4.52 (m, 2H), 3.48 (s, | |
| | | 2H), 3.23-3.18 (m, 2H), 1.17-1.14 | |
| | | (m, 2H), 1.06-1.03 (m, 2H). | |
| 330 | 2-(6-bromo-1-oxospiro[3H-isoquinoline- | ¹H NMR (400 MHz; DMSO-d₆): | m/z = |
| | 4,1′-cyclopropane]-2-yl)-N-(6,8-dihydro- | δ 10.72 (s, 1H), 8.47 (s, 1H), | 443.2, |
| | 5H-pyrano[3,4-d]pyrimidin-2- | 7.81 (d, J = 8.3 Hz, 1H), 7.53 (dd, | 445.2 |
| | yl)acetamide | J = 8.3, 1.9 Hz, 1H), 7.26 (d, J = | [M + H]⁺ |
| | | 1.9 Hz, 1H), 4.59 (s, 2H), 4.51 (s, | |
| | | 2H), 3.93-3.90 (m, 2H), 3.48 (s, | |
| | | 2H), 2.78-2.75 (m, 2H), 1.17-1.13 | |
| | | (m, 2H), 1.06-1.02 (m, 2H). | |
| 331 | 2-(6-bromo-1-oxospiro[3H-isoquinoline- | ¹H NMR (400 MHZ, DMSO-d₆): | m/z = |
| | 4,1′-cyclopropane]-2-yl)-N-(7,8-dihydro- | δ10.70 (s, 1H), 8.38 (s, 1H), 7.80 | 442.9, |
| | 5H-pyrano[4,3-d]pyrimidin-2- | (d, J = 8.4 Hz, 1H), 7.53-7.51 (dd, | 444.9 |
| | yl)acetamide | J = 2.0, 8.4 Hz, 1H), 7.26 (d, J = | [M + H]⁺ |
| | | 2.0 Hz, 1H), 4.68 (s, 2H), 4.52 (s, | |
| | | 2H), 3.97 (t, J = 5.6 Hz, 2H), 3.48 | |
| | | (s, 2H), 2.83 (t, J = 5.6 Hz, 2H), | |
| | | 1.17-1.14 (m, 2H), 1.06-1.03 (m, | |
| | | 2H). | |
| 332 | 2-(6-bromo-1-oxospiro[3H-isoquinoline- | ¹H NMR (400 MHZ, CDCl₃): δ | m/z = |
| | 4,1′-cyclopropane]-2-yl)-N-[5-(oxetan-3- | 9.05 (br s, 1H), 8.70 (s, 2H), 8.03 | 443.0, |
| | yl)pyrimidin-2-yl]acetamide | (d, J = 8.4 Hz, 1H), 7.47 (dd, J = | 445.0 |
| | | 1.6, 8.4 Hz, 1H), 7.08-6.98 (m, | [M + H]⁺ |
| | | 1H), 5.14 (dd, J = 6.4, 8.4 Hz, | |
| | | 2H), 4.76-4.63 (m, 4H), 4.24-4.16 | |
| | | (m, 1H), 3.56 (s, 2H), 1.18-1.13 | |
| | | (m, 2H), 1.12-1.07 (m, 2H). | |
| 333 | 2-(6-bromo-1-oxospiro[3H-isoquinoline- | ¹H NMR (400 MHZ, CDCl₃): δ | m/z = |
| | 4,1′-cyclopropane]-2-yl)-N-(6- | 8.94 (br s, 1H), 8.60 (d, J = 5.2 | 443.9. |
| | fluoropyrazolo[1,5-a]pyrimidin-5- | Hz, 1H), 8.10-7.96 (m, 2H), 7.47 | 445.9 |
| | yl)acetamide | (dd, J = 1.6, 8.0 Hz, 1H), 7.02 (d, | [M + H]⁺ |
| | | J = 1.6 Hz, 1H), 6.57 (d, J = 2.0 | |
| | | Hz, 1H), 4.67 (s, 2H), 3.57 (s, | |
| | | 2H), 1.17-1.07 (m, 4H). | |
| 334 | 2-(6-bromo-1-oxospiro[3H-isoquinoline- | | m/z = |
| | 4,1′-cyclopropane]-2-yl)-N-[3-cis-(1H- | | 429.0, |
| | pyrazol-5-yl)cyclobutyl]acetamide | | 431.0 |
| | | | [M + H]⁺ |
| 335 | 2-(6-bromo-1-oxospiro[3H-isoquinoline- | | m/z = |
| | 4,1′-cyclopropane]-2-yl)-N-(1-methyl-6- | | 417.2, |
| | oxopyridazin-3-yl)acetamide | | 419.2 |
| | | | [M + H]⁺ |
| 336 | N-(5-fluoropyrimidin-2-yl)-2-[6-(oxetan- | | m/z = |
| | 3-yl)-1-oxospiro[3H-isoquinoline-4,1′- | | 383.2 |
| | cyclopropane]-2-yl]acetamide | | [M + H]⁺ |
| 337 | 2-(6-bromo-1-oxospiro[3H-isoquinoline- | | m/z = |
| | 4,1′-cyclopropane]-2-yl)-N-(4-methyl-5- | | 416.9, |
| | oxopyrazin-2-yl)acetamide | | 418.9 |
| | | | [M + H]⁺ |
| 338 | 2-(6-bromo-1-oxospiro[3H-isoquinoline- | | m/z = |
| | 4,1′-cyclopropane]-2-yl)-N-(4- | | 417.2, |
| | methoxypyrimidin-2-yl)acetamide | | 419.2 |
| | | | [M + H]⁺ |
| 339 | 2-(6-bromo-1-oxospiro[3H-isoquinoline- | | m/z = |
| | 4,1′-cyclopropane]-2-yl)-N-(5- | | 419.0, |
| | fluoropyrimidin-2-yl)propanamide | | 421.0 |
| | | | [M + H]⁺ |
| 340 | 2-(6-bromo-1-oxospiro[3H-isoquinoline- | | m/z = |
| | 4,1′-cyclopropane]-2-yl)-N-(3-trans- | | 407.0, |
| | methoxy-3-methylcyclobutyl)acetamide | | 409.0 |
| | | | [M + H]⁺ |
| 341 | 2-(6-bromo-1-oxospiro[3H-isoquinoline- | | m/z = |
| | 4,1′-cyclopropane]-2-yl)-N-(3-cis- | | 407.0, |
| | methoxy-3-methylcyclobutyl)acetamide | | 409.0 |
| | | | [M + H]⁺ |
| 342 | 2-(6-bromo-1-oxospiro[3H-isoquinoline- | | m/z = |
| | 4,1′-cyclopropane]-2-yl)-N-(3-cis- | | 407.0, |
| | hydroxy-3-methylcyclobutyl)-N- | | 409.0 |
| | methylacetamide | | [M + H]⁺ |
| 343 | 2-(6-bromo-1-oxospiro[3H-isoquinoline- | | m/z = |
| | 4,1′-cyclopropane]-2-yl)-N-(3- | | 443.1, |
| | fluoroimidazo[1,2-a]pyridin-2- | | 445.1 |
| | yl)acetamide | | [M + H]⁺ |
| 344 | 2-[8-bromo-4-methyl-1-oxo-4- | | m/z = |
| | (trifluoromethyl)-3H-isoquinolin-2-yl]- | | 461.0, |
| | N-(5-fluoropyrimidin-2-yl)acetamide | | 463.1 |
| | | | [M + H]⁺ |
| 345 | 2-(6-bromo-1-oxospiro[3H-isoquinoline- | | m/z = |
| | 4,1′-cyclopropane]-2-yl)-N-(6-cyano-4- | | 428.9, |
| | fluoropyridin-3-yl)acetamide | | 430.9 |
| | | | [M + H]⁺ |
| 346 | 2-(6-bromo-1-oxospiro[3H-isoquinoline- | | m/z = |
| | 4,1′-cyclopropane]-2-yl)-N- | | 427.1, |
| | ([1,2,4]triazolo[1,5-a]pyrazin-8- | | 429.1 |
| | yl)acetamide | | [M + H]⁺ |
| 347 | 2-(6-bromo-1-oxospiro[3H-isoquinoline- | | m/z = |
| | 4,1′-cyclopropane]-2-yl)-N-(1-methyl-2- | | 406.3, |
| | oxopyrrolidin-3-yl)acetamide | | 408.2 |
| | | | [M + H]⁺ |
| 348 | 2-(6-bromo-1-oxospiro[3H-isoquinoline- | | m/z = |
| | 4,1′-cyclopropane]-2-yl)-N-(4-methyl-3- | | 416.9, |
| | oxopyrazin-2-yl)acetamide | | 418.9 |
| | | | [M + H]⁺ |
| 349 | 2-(6-bromo-1-oxospiro[3H-isoquinoline- | | m/z = |
| | 4,1′-cyclopropane]-2-yl)-N-(1,7- | | 437.3, |
| | naphthyridin-8-yl)acetamide | | 439.2 |
| | | | [M + H]⁺ |
| 350 | 2-(6-bromo-1-oxospiro[3H-isoquinoline- | | m/z = |
| | 4,1′-cyclopropane]-2-yl)-N- | | 427.2, |
| | ([1,2,4]triazolo[4,3-a]pyrazin-8- | | 429.1 |
| | yl)acetamide | | [M + H]⁺ |
| 351 | 2-(6-bromo-1-oxospiro[3H-isoquinoline- | | m/z = |
| | 4,1′-cyclopropane]-2-yl)-N-imidazo[1,2- | | 426.4, |
| | alpyrazin-8-ylacetamide | | 428.4 |
| | | | [M + H]⁺ |
| 352 | N-(1,2,4-benzotriazin-3-yl)-2-(6-bromo- | | m/z = |
| | 1-oxospiro[3H-isoquinoline-4,1′- | | 438.2, |
| | cyclopropane]-2-yl)acetamide | | 440.1 |
| | | | [M + H]⁺ |
| 353 | 2-(6-bromo-1-oxospiro[3H-isoquinoline- | | m/z = |
| | 4,1′-cyclopropane]-2-yl)-N-(5,5- | | 406.0, |
| | dimethyl-4H-1,3-oxazol-2-yl)acetamide | | 408.0 |
| | | | [M + H]⁺ |
| 354 | 2-[8-chloro-1-oxo-6- | | m/z = |
| | (trifluoromethyl)spiro[3H-isoquinoline- | | 429.0, |
| | 4,1′-cyclopropane]-2-yl]-N-(5- | | 431.0 |
| | fluoropyrimidin-2-yl)acetamide | | [M + H]⁺ |
| 355 | N-[(3R)-1-benzylpyrrolidin-3-yl]-2-(6- | | m/z = |
| | bromo-1-oxospiro[3H-isoquinoline-4,1′- | | 468.3, |
| | cyclopropane]-2-yl)acetamide | | 470.3 |
| | | | [M + H]⁺ |
| 356 | 2-(6-bromo-1-oxospiro[3H-isoquinoline- | | m/z = |
| | 4,1′-cyclopropane]-2-yl)-N-(4-cyano-1,3- | | 451.2, |
| | benzoxazol-2-yl)acetamide | | 453.2 |
| | | | [M + H]⁺ |
| 357 | 2-(6-bromo-1-oxospiro[3H-isoquinoline- | ¹H NMR (400 MHZ, DMSO-d₆): | |
| | 4,1′-cyclopropane]-2-yl)-N-(3-trans- | δ 8.32 (d, J = 7.0 Hz, 1H), 7.80 | |
| | phenylmethoxycyclobutyl)acetamide | (d, J = 8.3 Hz, 1H), 7.51 (dd, J = | |
| | | 8.3, 1.9 Hz, 1H), 7.37-7.24 (m, | |
| | | 5H), 4.37 (s, 2H), 4.25-4.23 (m, | |
| | | 1H), 4.17-4.14 (m, 1H), 4.08 (s, | |
| | | 2H), 3.43 (s, 2H), 2.29-2.23 (m, | |
| | | 2H), 2.16-2.10 (m, 2H), 1.15-1.12 | |
| | | (m, 2H), 1.04-1.01 (m, 2H) | |
| 358 | 2-(6-bromo-1-oxospiro[3H-isoquinoline- | | m/z = |
| | 4,1′-cyclopropane]-2-yl)-N-[5- | | 494.2, |
| | (trifluoromethyl)-1,3-benzoxazol-2- | | 496.2 |
| | yl]acetamide | | [M + H]⁺ |
| 359 | 2-(6-bromo-1-oxospiro[3H-isoquinoline- | | m/z = |
| | 4,1′-cyclopropane]-2-yl)-N-[5-(oxolan-2- | | 456.9, |
| | yl)pyrimidin-2-yl]acetamide | | 459.0 |
| | | | [M + H]⁺ |
| 360 | 2-(6-bromo-1-oxospiro[3H-isoquinoline- | | m/z = |
| | 4,1′-cyclopropane]-2-yl)-N-[(3R)-1- | | 406.5, |
| | methylpiperidin-3-yl]acetamide | | 408.4 |
| | | | [M + H]⁺ |
| 361 | 2-(6-bromo-1-oxospiro[3H-isoquinoline- | ¹H NMR (400 MHZ, DMSO-d₆): | m/z = |
| | 4,1′-cyclopropane]-2-yl)-N-[6- | δ 12.26 (s, 1H), 8.12 (s, 1H), 7.82 | 494.2, |
| | (trifluoromethyl)-1,3-benzoxazol-2- | (d, J = 8.3 Hz, 1H), 7.78-7.76 (m, | 496.1 |
| | yl]acetamide | 1H), 7.69-7.67 (m, 1H), 7.54 | [M + H]⁺ |
| | | (dd, J = 8.3, 1.9 Hz, 1H), 7.28 | |
| | | (d, J = 1.9 Hz, 1H), 4.57 (s, 2H), | |
| | | 3.53 (s, 2H), 1.20-1.16 (m, 2H), | |
| | | 1.08-1.05 (m, 2H) | |
| 362 | 2-(6-bromo-1-oxospiro[3H-isoquinoline- | | m/z = |
| | 4,1′-cyclopropane]-2-yl)-N-(6,7-dihydro- | | 431.2, |
| | 4H-pyrazolo[5,1-c][1,4]oxazin-2- | | 433.2 |
| | yl)acetamide | | [M + H]⁺ |
| 363 | 2-(6-bromo-1-oxospiro[3H-isoquinoline- | | m/z = |
| | 4,1′-cyclopropane]-2-yl)-N-imidazo[1,2- | | 426.0, |
| | c]pyrimidin-5-ylacetamide | | 428.0 |
| | | | [M + H]⁺ |
| 364 | 2-(6-bromo-1-oxospiro[3H-isoquinoline- | | m/z = |
| | 4,1′-cyclopropane]-2-yl)-N-(5-pyrrolidin- | | 456.0, |
| | 1-ylpyrimidin-2-yl)acetamide | | 458.0 |
| | | | [M + H]⁺ |
| 365 | 2-(6-bromo-1-oxospiro[3H-isoquinoline- | | m/z = |
| | 4,1′-cyclopropane]-2-yl)-N-[(3R)-1- | | 448.4, |
| | (oxetan-3-yl)piperidin-3-yl]acetamide | | 450.4 |
| | | | [M + H]⁺ |
| 366 | 2-(6-bromo-1-oxospiro[3H-isoquinoline- | | m/z = |
| | 4,1′-cyclopropane]-2-yl)-N-[1-(3,3,3- | | 488.3, |
| | trifluoropropyl)piperidin-4-yl]acetamide | | 490.3 |
| | | | [M + H]⁺ |
| 367 | 2-(6-bromo-1-oxospiro[3H-isoquinoline- | | m/z = |
| | 4,1′-cyclopropane]-2-yl)-N-[1-(2,2- | | 456.3, |
| | difluoroethyl)piperidin-4-yl]acetamide | | 458.3 |
| | | | [M + H]⁺ |
| 368 | 2-(6-bromo-1-oxospiro[3H-isoquinoline- | | m/z = |
| | 4,1′-cyclopropane]-2-yl)-N-(7- | | 440.2, |
| | methylpyrazolo[1,5-a]pyrimidin-5- | | 442.2 |
| | yl)acetamide | | [M + H]⁺ |
| 369 | 2-(6-bromo-1-oxospiro[3H-isoquinoline- | | m/z = |
| | 4,1′-cyclopropane]-2-yl)-N-(1-pyridin-3- | | 469.3, |
| | ylpiperidin-4-yl)acetamide | | 471.3 |
| | | | [M + H]⁺ |
| 370 | 2-(6-bromo-1-oxospiro[3H-isoquinoline- | | m/z = |
| | 4,1′-cyclopropane]-2-yl)-N-(4-methyl-4- | | 432.4, |
| | azaspiro[2.5]octan-7-yl)acetamide | | 434.4 |
| | | | [M + H]⁺ |
| 371 | 2-(6-bromo-1-oxospiro[3H-isoquinoline- | | m/z = |
| | 4,1′-cyclopropane]-2-yl)-N-(1-methyl- | | 390.2, |
| | 1,2,4-triazol-3-yl)acetamide | | 392.2 |
| | | | [M + H]⁺ |
| 372 | N-(1-azabicyclo[2.2.2]octan-3-yl)-2-(6- | | m/z = |
| | bromo-1-oxospiro[3H-isoquinoline-4,1′- | | 418.2, |
| | cyclopropane]-2-yl)acetamide | | 420.2 |
| | | | [M + H]⁺ |
| 373 | 2-(6-bromo-1-oxospiro[3H-isoquinoline- | | m/z = |
| | 4,1′-cyclopropane]-2-yl)-N-(5,6,7,8- | | 430.3, |
| | tetrahydro-[1,2,4]triazolo[1,5-a]pyridin- | | 432.3 |
| | 2-yl)acetamide | | [M + H]⁺ |
| 374 | 2-(6-bromo-1-oxospiro[3H-isoquinoline- | | m/z = |
| | 4,1′-cyclopropane]-2-yl)-N-imidazo[1,2- | | 425.9, |
| | a]pyrimidin-7-ylacetamide | | 428.0 |
| | | | [M + H]⁺ |
| 375 | 2-(6-bromo-1-oxospiro[3H-isoquinoline- | | m/z = |
| | 4,1′-cyclopropane]-2-yl)-N-[1-(2,2,2- | | 474.3, |
| | trifluoroethyl)piperidin-4-yl]acetamide | | 476.3 |
| | | | [M + H]⁺ |
| 376 | 2-(6-bromo-1-oxospiro[3H-isoquinoline- | | m/z = |
| | 4,1′-cyclopropane]-2-yl)-N-(1,5- | | 404.2, |
| | dimethyl-1,2,4-triazol-3-yl)acetamide | | 406.2 |
| | | | [M + H]⁺ |
| 377 | N-(1,2-benzoxazol-3-yl)-2-(6-bromo-1- | | m/z = |
| | oxospiro[3H-isoquinoline-4,1′- | | 426.2, |
| | cyclopropane]-2-yl)acetamide | | 428.2 |
| | | | [M + H]⁺ |
| 378 | ethyl 4-[[2-(6-bromo-1-oxospiro[3H- | ¹H NMR (400 MHZ, DMSO-d₆): | |
| | isoquinoline-4,1′-cyclopropane]-2- | δ 8.00-7.97 (m, 1H), 7.81 (d, J = | |
| | yl)acetyl]amino ]piperidine-1-carboxylate | 8.3 Hz, 1H), 7.51 (dd, J = 8.3, 1.9 | |
| | | Hz, 1H), 7.24 (d, J = 1.8 Hz, 1H), | |
| | | 4.10 (d, J = 3.0 Hz, 2H), 4.03 (q, | |
| | | J = 7.1 Hz, 2H), 3.90-3.85 (m, | |
| | | 2H), 3.79-3.71 (m, 1H), 3.43 (s, | |
| | | 2H), 2.94-2.87 (m, 2H), 1.74-1.70 | |
| | | (m, 2H), 1.33-1.22 (m, 2H), 1.20- | |
| | | 1.12 (m, 5H), 1.04-1.01 (m, 2H) | |
| 379 | 2-(6-bromo-1-oxospiro[3H-isoquinoline- | | m/z = |
| | 4,1′-cyclopropane]-2-yl)-N-(1-pyridin-2- | | 469.3, |
| | ylpiperidin-4-yl)acetamide | | 471.3 |
| | | | [M + H]⁺ |
| 380 | 2-(6-bromo-1-oxospiro[3H-isoquinoline- | | m/z = |
| | 4,1′-cyclopropane]-2-yl)-N-(1-pyrimidin- | | 470.3, |
| | 2-ylpiperidin-4-yl)acetamide | | 472.3 |
| | | | [M + H]⁺ |
| 381 | 2-(6-bromo-1-oxospiro[3H-isoquinoline- | | m/z = |
| | 4,1′-cyclopropane]-2-yl)-N-[(3R)-1- | | 434.3, |
| | (oxetan-3-yl)pyrrolidin-3-yl]acetamide | | 436.3 |
| | | | [M + H]⁺ |
| 382 | 2-(6-bromo-1-oxospiro[3H-isoquinoline- | | m/z = |
| | 4,1′-cyclopropane]-2-yl)-N-[3- | | 407.3, |
| | (methoxymethyl)cyclobutyl]acetamide | | 409.3 |
| | | | [M + H]⁺ |
| 383 | 2-(6-bromo-1-oxospiro[3H-isoquinoline- | | m/z = |
| | 4,1′-cyclopropane]-2-yl)-N-pyrazolo[1,5- | | 425.2, |
| | alpyridin-2-ylacetamide | | 427.2 |
| | | | [M + H]⁺ |
| 384 | 2-(6-bromo-1-oxospiro[3H-isoquinoline- | | m/z = |
| | 4,1′-cyclopropane]-2-yl)-N-[(3R)-1- | | 406.5, |
| | ethylpyrrolidin-3-yl]acetamide | | 408.5 |
| | | | [M + H]⁺ |
| 385 | 2-(6-bromo-1-oxospiro[3H-isoquinoline- | | m/z = |
| | 4,1′-cyclopropane]-2-yl)-N-imidazo[1,2- | | 425.2, |
| | alpyridin-2-ylacetamide | | 427.2 |
| | | | [M + H]⁺ |
| 386 | 2-(6-bromo-1-oxospiro[3H-isoquinoline- | | m/z = |
| | 4,1′-cyclopropane]-2-yl)-N-(4,5,6,7- | | 430.2, |
| | tetrahydro-1,3-benzoxazol-2- | | 432.3 |
| | yl)acetamide | | [M + H]⁺ |
| 387 | 2-(6-bromo-1-oxospiro[3H-isoquinoline- | | m/z = |
| | 4,1′-cyclopropane]-2-yl)-N-(1-pyridazin- | | 470.3, |
| | 3-ylpiperidin-4-yl)acetamide | | 472.2 |
| | | | [M + H]⁺ |
| 389 | 2-(6-bromo-1-oxospiro[3H-isoquinoline- | ¹H NMR (400 MHZ, DMSO-d₆): | m/z = |
| | 4,1′-cyclopropane]-2-yl)-N-(5-cyano-3- | δ 10.90 (br s, 1H), 8.64 (s, 1H), | 443.0, |
| | fluoro-4-methylpyridin-2-yl)acetamide | 7.81 (d, J = 8.4 Hz, 1H), 7.52 (dd, | 444.9 |
| | | J = 1.6, 8.4 Hz, 1H), 7.26 (d, J = | [M + H]⁺ |
| | | 1.6 Hz, 1H), 4.48 (s, 2H), 3.50 (s, | |
| | | 2H), 2.43 (d, J = 1.2 Hz, 3H), | |
| | | 1.20-1.11 (m, 2H), 1.09-1.00 (m, | |
| | | 2H). | |
| 390 | 2-(6-bromo-1-oxospiro[3H-isoquinoline- | | m/z = |
| | 4,1′-cyclopropane]-2-yl)-N-(5-cyano-3- | | 443.0, |
| | fluoro-6-methylpyridin-2-yl)acetamide | | 445.0 |
| | | | [M + H]⁺ |
| 391 | 2-(6-bromo-1-oxospiro[3H-isoquinoline- | | m/z = |
| | 4,1′-cyclopropane]-2-yl)-N-(5-fluoro-2- | | 435.2, |
| | methoxypyrimidin-4-yl)acetamide | | 437.2 |
| | | | [M + H]⁺ |
| 392 | 2-(6-bromo-1-oxospiro[3H-isoquinoline- | | m/z = |
| | 4,1′-cyclopropane]-2-yl)-N-(3-cyano-5- | | 429.2, |
| | fluoropyridin-2-yl)acetamide | | 431.2 |
| | | | [M + H]⁺ |
| 393 | 2-(6-bromo-1-oxospiro[3H-isoquinoline- | | m/z = |
| | 4,1′-cyclopropane]-2-yl)-N-(1-ethyl-1- | | 432.4, |
| | azaspiro[3.3]heptan-6-yl)acetamide | | 434.3 |
| | | | [M + H]⁺ |
| 394 | tert-butyl 6-[[2-(6-bromo-1-oxospiro[3H- | | m/z = |
| | isoquinoline-4,1′-cyclopropane]-2- | | 504.4, |
| | yl)acetyl]amino]-1-azaspiro[3.3]heptane- | | 506.3 |
| | 1-carboxylate | | [M + H]⁺ |
| 395 | 2-(6-bromo-1-oxospiro[3H-isoquinoline- | | m/z = |
| | 4,1′-cyclopropane]-2-yl)-N-(3-chloro-5- | | 445.2, |
| | cyanopyridin-2-yl)acetamide | | 447.2, |
| | | | 449.1 |
| | | | [M + H]⁺ |
| 396 | 2-(6-bromo-1-oxospiro[3H-isoquinoline- | | m/z = |
| | 4,1′-cyclopropane]-2-yl)-N-(3-fluoro-5- | | 418.2, |
| | methylpyridin-2-yl)acetamide | | 420.2 |
| | | | [M + H]⁺ |
| 397 | 2-(6-bromo-1-oxospiro[3H-isoquinoline- | | m/z = |
| | 4,1′-cyclopropane]-2-yl)-N-(5-cyano-3- | | 425.2, |
| | methylpyridin-2-yl)acetamide | | 427.2 |
| | | | [M + H]⁺ |
| 398 | N-(1,2-benzothiazol-6-yl)-2-(6-bromo-1- | | m/z = |
| | oxospiro[3H-isoquinoline-4,1′- | | 442.2, |
| | cyclopropane]-2-yl)acetamide | | 444.2 |
| | | | [M + H]⁺ |
| 399 | 2-(6-bromo-1-oxospiro[3H-isoquinoline- | | m/z = |
| | 4,1′-cyclopropane]-2-yl)-N-(3- | | 440.3, |
| | methylimidazo[1,2-b]pyridazin-6- | | 442.3 |
| | yl)acetamide | | [M + H]⁺ |
| 400 | 2-(6-bromo-1-oxospiro[3H-isoquinoline- | | m/z = |
| | 4,1′-cyclopropane]-2-yl)-N-(2- | | 440.2, |
| | methylimidazo[1,2-b]pyridazin-6- | | 442.2 |
| | yl)acetamide | | [M + H]⁺ |
| 401 | 2-(6-bromo-1-oxospiro[3H-isoquinoline- | | m/z = |
| | 4,1′-cyclopropane]-2-yl)-N-(5-fluoro-6- | | 435.0, |
| | methoxypyrimidin-4-yl)acetamide | | 437.0 |
| | | | [M + H]⁺ |
| 402 | 2-(6-bromo-1-oxospiro[3H-isoquinoline- | | m/z = |
| | 4,1′-cyclopropane]-2-yl)-N-(3- | | 439.0, |
| | methylpyrazolo[1,5-a]pyridin-2- | | 440.9 |
| | yl)acetamide | | [M + H]⁺ |
| 403 | 2-(6-bromo-1-oxospiro[3H-isoquinoline- | | m/z = |
| | 4,1′-cyclopropane]-2-yl)-N-(3- | | 443.0, |
| | fluoropyrazolo[1,5-a]pyridin-2- | | 445.0 |
| | yl)acetamide | | [M + H]⁺ |
| 404 | N-(5-fluoropyrimidin-2-yl)-2-[6-[(1r,2r)- | | m/z = |
| | 1-fluoro-2-(trifluoromethyl)cyclopropyl]- | | 453.0 |
| | 1-oxospiro[3H-isoquinoline-4,1′- | | [M + H]⁺ |
| | cyclopropane]-2-yl]acetamide | | |
| 405 | 2-(6-bromo-1-oxospiro[3H-isoquinoline- | | m/z = |
| | 4,1′-cyclopropane]-2-yl)-N-(4,4- | | 406.3, |
| | dimethyl-5H-1,3-oxazol-2-yl)acetamide | | 408.3 |
| | | | [M + H]⁺ |
| 406 | 2-(6-bromo-1-oxospiro[3H-isoquinoline- | | m/z = |
| | 4,1′-cyclopropane]-2-yl)-N-(2- | | 392.2, |
| | oxopyrrolidin-3-yl)acetamide | | 394.2 |
| | | | [M + H]⁺ |
| 407 | 2-(6-bromo-1-oxospiro[3H-isoquinoline- | | m/z = |
| | 4,1′-cyclopropane]-2-yl)-N-(3- | | 416.0, |
| | methoxypyridin-2-yl)acetamide | | 418.0 |
| | | | [M + H]⁺ |
| 408 | 2-(6-bromo-1-oxospiro[3H-isoquinoline- | | m/z = |
| | 4,1′-cyclopropane]-2-yl)-N-(1- | | 441.0, |
| | methylpyrazolo[3,4-d]pyrimidin-6- | | 443.0 |
| | yl)acetamide | | [M + H]⁺ |
| 409 | 2-(6-bromo-1-oxospiro[3H-isoquinoline- | | m/z = |
| | 4,1′-cyclopropane]-2-yl)-N- | | 443.9, |
| | ([1,3]thiazolo[5,4-d]pyrimidin-5- | | 445.9 |
| | yl)acetamide | | [M + H]⁺ |
| 410 | 2-(6-bromo-1-oxospiro[3H-isoquinoline- | | m/z = |
| | 4,1′-cyclopropane]-2-yl)-N-pyridazin-4- | | 387.2, |
| | ylacetamide | | 389.2 |
| | | | [M + H]⁺ |
| 411 | 2-(6-bromo-1-oxospiro[3H-isoquinoline- | | m/z = |
| | 4,1′-cyclopropane]-2-yl)-N-pyrazin-2- | | 387.2, |
| | ylacetamide | | 389.2 |
| | | | [M + H]⁺ |
| 412 | 2-(6-bromo-1-oxospiro[3H-isoquinoline- | | m/z = |
| | 4,1′-cyclopropane]-2-yl)-N-(5-methyl-1- | | 466.3, |
| | phenyl-1,2,4-triazol-3-yl)acetamide | | 468.3 |
| | | | [M + H]⁺ |
| 413 | 2-(6-bromo-1-oxospiro[3H-isoquinoline- | | m/z = |
| | 4,1′-cyclopropane]-2-yl)-N-(1-phenyl- | | 452.2, |
| | 1,2,4-triazol-3-yl)acetamide | | 454.4 |
| | | | [M + H]⁺ |
| 414 | 2-(6-bromo-1-oxospiro[3H-isoquinoline- | | m/z = |
| | 4,1′-cyclopropane]-2-yl)-N-(6- | | 411.0, |
| | cyanopyridin-3-yl)acetamide | | 413.0 |
| | | | [M + H]⁺ |
| 415 | 2-(6-bromo-1-oxospiro[3H-isoquinoline- | | m/z = |
| | 4,1′-cyclopropane]-2-yl)-N-[1-(1- | | 458.3, |
| | methylpyrazol-3-yl)pyrrolidin-3- | | 460.2 |
| | yl]acetamide | | [M + H]⁺ |
| 416 | 2-(6-bromo-1-oxospiro[3H-isoquinoline- | | m/z = |
| | 4,1′-cyclopropane]-2-yl)-N-[1-(1- | | 458.4, |
| | methylpyrazol-4-yl)pyrrolidin-3- | | 460.2 |
| | yl]acetamide | | [M + H]⁺ |
| 417 | 2-(6-bromo-1-oxospiro[3H-isoquinoline- | | m/z = |
| | 4,1′-cyclopropane]-2-yl)-N-[1-(1- | | 472.3, |
| | methylpyrazol-4-yl)piperidin-3- | | 474.3 |
| | yl]acetamide | | [M + H]⁺ |
| 418 | 2-(6-bromo-1-oxospiro[3H-isoquinoline- | | m/z = |
| | 4,1′-cyclopropane]-2-yl)-N-(6- | | 416.0, |
| | methoxypyridin-3-yl)acetamide | | 417.9 |
| | | | [M + H]⁺ |
| 419 | 2-(6-bromo-1-oxospiro[3H-isoquinoline- | | m/z = |
| | 4,1′-cyclopropane]-2-yl)-N-[5- | | 453.9, |
| | (difluoromethyl)-3-fluoropyridin-2- | | 455.9 |
| | yl]acetamide | | [M + H]⁺ |
| 420 | 2-(6-bromo-1-oxospiro[3H-isoquinoline- | | m/z = |
| | 4,1′-cyclopropane]-2-yl)-N-[6- | | 452.4, |
| | (difluoromethoxy)pyridin-3-yl]acetamide | | 454.3 |
| | | | [M + H]⁺ |
| 421 | 2-(6-bromo-1-oxospiro[3H-isoquinoline- | | m/z = |
| | 4,1′-cyclopropane]-2-yl)-N-(1-propan-2- | | 418.5, |
| | yl-1,2,4-triazol-3-yl)acetamide | | 420.5 |
| | | | [M + H]⁺ |
| 422 | 2-(6-bromo-1-oxospiro[3H-isoquinoline- | | m/z = |
| | 4,1′-cyclopropane]-2-yl)-N-(1- | | 416.2, |
| | cyclopropyl-1,2,4-triazol-3-yl)acetamide | | 418.2 |
| | | | [M + H]⁺ |
| 423 | 2-[6-(cyclopropylmethyl)-1-oxospiro[3H- | | m/z = |
| | isoquinoline-4,1′-cyclopropane]-2-yl]-N- | | 381.2 |
| | (5-fluoropyrimidin-2-yl)acetamide | | [M + H]⁺ |
| 424 | 2-(6-bromo-1-oxospiro[3H-isoquinoline- | | m/z = |
| | 4,1′-cyclopropane]-2-yl)-N-[5-(2,3- | | 455.0, |
| | dihydrofuran-4-yl)pyrimidin-2- | | 457.0 |
| | yl]acetamide | | [M + H]⁺ |
| 425 | 2-(6-bromo-1-oxospiro[3H-isoquinoline- | | m/z = |
| | 4,1′-cyclopropane]-2-yl)-N-[1-(2,2,2- | | 458.3, |
| | trifluoroethyl)-1,2,4-triazol-3- | | 460.2 |
| | yl]acetamide | | [M + H]⁺ |
| 427 | 2-(6-bromo-1-oxospiro[3H-isoquinoline- | | m/z = |
| | 4,1′-cyclopropane]-2-yl)-N-(3-trans- | | 439.4, |
| | phenylcyclobutyl)acetamide | | 441.4 |
| | | | [M + H]⁺ |
| 429 | 2-(6-bromo-1-oxospiro[3H-isoquinoline- | | m/z = |
| | 4,1′-cyclopropane]-2-yl)-N-(8-chloro- | | 460.1, |
| | [1,2,4]triazolo[1,5-a]pyridin-2- | | 462.1 |
| | yl)acetamide | | [M + H]⁺ |
| 430 | 2-(6-bromo-1-oxospiro[3H-isoquinoline- | | LCMS |
| | 4,1′-cyclopropane]-2-yl)-N-(6-oxo-1H- | | m/z = |
| | pyridazin-3-yl)acetamide | | 402.9, |
| | | | 404.9 |
| | | | [M + H]⁺ |

### Biological Example 1

### Biochemical Assay of the Compounds

### Procedure for Culturing THP-1 Cells

Compounds as provided herein were tested in the following assay. Cell culture medium employed contained RPMI 1640 medium (89%), FBS (10%), Pen/Strep (1%), and 2-mercaptoethanol (0.05 mM). Freezing medium was made up of 90% FBS and 10% DMSO. THP-1 cells were removed from the liquid nitrogen and placed into a 37° C. water bath to thaw, until signs of ice dissipated. The cells were then added to 9 mL of warm cell culture medium and centrifuged for 5 minutes at 1000 rpm. The supernatant was discarded, and the cells were resuspended in new cell culture medium. THP-1 cells were then split and cultured in the cell culture medium, being passaged every 2-3 days with the cell density will be maintained between 5×10⁵ and 1.5×10⁶ viable cells/mL.

To freeze, cells were resuspended with fresh freezing medium, adjusting the cell density to 5×10⁶ cells/mL. The cell suspension was partitioned into 1 mL aliquots per vial, and the vials were transferred to a −80° C. freezer. After one day at −80° C., the cell vials were transferred to liquid nitrogen freezer for storage.

### Procedure for IL-1β Secretion Assay in 384-Well Plates

PMA was dissolved in DMSO to make a stock solution at 5 mg/mL and stored in 10 μl aliquots at −20° C. for single use. PMA is added to normal growth medium. LPS was diluted with 1 mL of water solution to provide a 1 mg/mL stock solution and stored in 15 μl aliquots at −20° C. for single use. Nigericin is diluted in ice cold 100% ethanol to 5 mg/mL (6.7 mM) and stored in 75 μL aliquots at −20° C. for single use. Serum-free media contains RPMI 1640 medium (99%), Pen/Strep (1%), and 2-mercaptoethanol (0.05 mM). The two control conditions used to qualify and normalize test compound dose-response curves were as follows: High Control=25 ng/mL LPS, 5 μM Nigericin, 0.5% DMSO, Low Control=25 ng/mL, LPS, 0.5% DMSO.

Day 1: Differentiation with PMA

THP-1 cells were diluted to provide a suspension at a concentration of 1.0×10⁶ cells/mL with the total volume of suspension required to enable the desired number of assay plates. The growth media was supplemented with PMA (5 ng/mL final concentration) and the cells were incubated at 37° C. under a humidified atmosphere of 5% CO₂ for 40 h.

Day 3: Plating with Sequential LPS and Nigericin Stimulation

All media was carefully aspirated from each culture flask. The cells were washed carefully with 1×DPBS. The cells were then briefly digested with trypsin LE for 5 minutes at 23° C., and immediately resuspended in cell growth media. After resuspension, the cells were centrifuged at 1000 rpm for 3 minutes and the supernatant was discarded. The cells were resuspended in DPBS and once again centrifuged at 1000 rpm for 5 minutes. The supernatant was discarded and the cell pellet was resuspended in serum-free media supplemented with LPS (25 ng/mL final concentration) to enable the distribution of 30K THP-1 cells within 45 μL of media into each well of 384-well PDL-coated plates. The 384-well plates were then incubated at 37° C. under a humidified atmosphere of 5% CO₂ for 2 h. Following this period, test compounds were dispensed by Tecan across the desired concentration range with all wells normalized to a final 0.5% DMSO concentration. The plates were then incubated at 37° C. under a humidified atmosphere of 5% CO₂ for 1 h. Following this period, 5 μL of the 5 mg/mL nigericin stock solution was added to each of the appropriate wells and plates were centrifuged at 1000 rpm for 30 seconds. The plates were the immediately reintroduced to the incubator at 37° C. under a humidified atmosphere of 5% CO₂ for 2 h. After this time, 35 μL/well of supernatant was collected and transferred into v-bottom plate and centrifuged at 1000 rpm for 1 minute. These supernatant aliquots were analyzed using an IL-Iβ detection kit as described below. If needed, the test samples could be snap frozen and stored at −80° C. until analyzed.

### IL-1β Detection

To prepare each ELISA plate, capture antibody (mAb Mt175) was diluted with PBS to a final concentration of 2 μg/mL and then 20 μL of this solution was added to each well of the ELISA plate. Each plate was allowed to incubate overnight at 4° C. The next day, the capture antibody solution was removed and discarded. Each ELISA plate was washed 4 times with PBST followed by the addition of 25 μL/well of blocking buffer (Licor-927-40010) supplemented with 0.1% Tween 20. Each ELISA plate was then allowed to incubate for 1 hour at 23° C. After this time, the blocking buffer was removed and discarded. Each ELISA plate was washed 4 times with PBST. During this time, the v-bottomed plates containing the supernatant aliquots from the assay run were centrifuged at 300 g for 5 minutes before transferring 15 μL/well of the supernatant sample to each ELISA plate. Each ELISA plate was then allowed to incubate for 2 h at 23° C. After this time, the supernatant samples were removed and discarded. Each ELISA plate was washed 4 times with PBST. To each ELISA plate was added 15 μL/well of mAb7P10-biotin at 0.5 μg/mL (1:1000 diluted in blocking buffer). Each ELISA plate was then allowed to incubate for 1 h at 23° C. After this time, the antibody solution was removed and discarded. Each ELISA plate was washed 4 times with PBST. To each ELISA plate was added 20 μL/well of streptavidin-HRP (1:2000 diluted in blocking buffer). Each ELISA plate was then allowed to incubate for 1 h at 23° C. After this time, the buffer was removed and discarded. Each ELISA plate was washed 4 times with PBST. To each ELISA plate was added 20 L/well of HRP substrate. Each ELISA plate was then allowed to incubate for 2 minutes at 23° C. After this time, to each ELISA plate was added 40 μL/well of stop solution. Each ELISA plate was centrifuged at 1200 rpm for 30 seconds.

The plate was then read at 450 nm in a microplate reader. Percent inhibition was calculated as follows:

% inhibition rate=(treated samples−high control)/(low control−high control)×100

Activity of the tested compounds is provided in Table 3 and Table 4. In Table 3, activity is reported as follows: +++=IC₅₀<10 μM; ++=IC₅₀ 10-15 μM; +=IC₅₀>15 μM.

TABLE 3

| | Activity |
|---|---|
| Ex. | (μM) |
| 1 | +++ |
| 2 | +++ |
| 3 | +++ |
| 4 | +++ |
| 5 | +++ |
| 6 | +++ |
| 7 | +++ |
| 8 | +++ |
| 9 | +++ |
| 10 | +++ |
| 11 | +++ |
| 12 | +++ |
| 13 | +++ |
| 14 | +++ |
| 15 | +++ |
| 16 | +++ |
| 17 | +++ |
| 18 | +++ |
| 19 | +++ |
| 20 | +++ |
| 21 | +++ |
| 22 | ++ |
| 23 | +++ |
| 24 | +++ |
| 25 | +++ |
| 26 | +++ |
| 27 | +++ |
| 28 | +++ |
| 29 | +++ |
| 30 | + |
| 31 | +++ |
| 32 | +++ |
| 33 | +++ |
| 34 | +++ |
| 35 | +++ |
| 36 | +++ |
| 37 | +++ |
| 38 | +++ |
| 39 | +++ |
| 40 | +++ |
| 41 | +++ |
| 42 | +++ |
| 43 | +++ |
| 44 | +++ |
| 45 | +++ |
| 46 | +++ |
| 47 | +++ |
| 48 | +++ |
| 49 | +++ |
| 50 | +++ |
| 51 | ++ |
| 52 | +++ |
| 53 | +++ |
| 54 | +++ |
| 55 | +++ |
| 56 | +++ |
| 57 | +++ |
| 58 | +++ |
| 59 | + |
| 60 | +++ |
| 61 | +++ |
| 62 | ++ |
| 63 | + |
| 64 | +++ |
| 65 | +++ |
| 66 | + |
| 67 | +++ |
| 68 | + |
| 69 | +++ |
| 70 | +++ |
| 71 | +++ |
| 72 | +++ |
| 73 | ++ |
| 74 | +++ |
| 75 | ++ |
| 76 | + |
| 77 | +++ |
| 78 | +++ |
| 79 | +++ |
| 80 | +++ |
| 81 | +++ |
| 82 | +++ |
| 83 | +++ |
| 84 | +++ |
| 85 | +++ |
| 86 | +++ |
| 87 | +++ |
| 88 | +++ |
| 89 | +++ |
| 90 | +++ |
| 91 | +++ |
| 92 | +++ |
| 93 | +++ |
| 94 | +++ |
| 95 | +++ |
| 96 | +++ |
| 97 | +++ |
| 98 | +++ |
| 99 | +++ |
| 100 | +++ |
| 101 | +++ |
| 102 | +++ |
| 103 | + |
| 104 | +++ |
| 105 | +++ |
| 106 | +++ |
| 107 | +++ |
| 108 | +++ |
| 109 | +++ |
| 110 | +++ |
| 111 | +++ |
| 112 | + |
| 113 | +++ |
| 114 | +++ |
| 115 | + |
| 116 | +++ |
| 117 | + |
| 118 | + |
| 119 | + |
| 120 | + |
| 121 | ++ |
| 122 | + |
| 123 | + |
| 124 | + |
| 125 | + |
| 126 | + |
| 127 | +++ |
| 128 | ++ |
| 129 | +++ |
| 130 | +++ |
| 131 | +++ |
| 132 | + |
| 133 | +++ |
| 134 | + |
| 135 | +++ |
| 136 | +++ |
| 137 | + |
| 138 | + |
| 139 | + |
| 140 | +++ |
| 141 | +++ |
| 142 | +++ |
| 143 | +++ |
| 144 | +++ |
| 145 | +++ |
| 146 | +++ |
| 147 | +++ |
| 148 | +++ |
| 149 | +++ |
| 150 | +++ |
| 151 | +++ |
| 152 | + |
| 153 | + |
| 154 | +++ |
| 155 | +++ |
| 156 | +++ |
| 157 | +++ |
| 158 | +++ |
| 159 | +++ |
| 160 | +++ |
| 161 | +++ |
| 162 | +++ |
| 163 | +++ |
| 164 | +++ |
| 165 | +++ |
| 166 | +++ |
| 167 | +++ |
| 168 | +++ |
| 169 | +++ |
| 170 | +++ |
| 171 | ++ |
| 172 | + |
| 173 | +++ |
| 174 | ++ |
| 175 | +++ |
| 176 | +++ |
| 177 | +++ |
| 178 | +++ |
| 179 | +++ |
| 180 | +++ |
| 181 | +++ |
| 182 | +++ |
| 183 | +++ |
| 184 | +++ |
| 185 | +++ |
| 186 | +++ |
| 187 | +++ |
| 188 | +++ |
| 189 | +++ |
| 190 | +++ |
| 191 | +++ |
| 192 | +++ |
| 193 | +++ |
| 194 | +++ |
| 195 | +++ |
| 196 | +++ |
| 197 | +++ |
| 198 | +++ |
| 199 | +++ |
| 200 | +++ |
| 201 | +++ |
| 202 | +++ |
| 203 | +++ |
| 204 | +++ |
| 205 | +++ |
| 206 | +++ |
| 207 | +++ |
| 208 | +++ |
| 209 | +++ |
| 210 | +++ |
| 211 | +++ |
| 212 | +++ |
| 213 | +++ |
| 214 | +++ |
| 215 | +++ |
| 216 | +++ |
| 217 | +++ |
| 218 | +++ |
| 219 | +++ |
| 220 | +++ |
| 221 | +++ |
| 222 | +++ |
| 223 | +++ |
| 224 | +++ |
| 225 | +++ |
| 226 | +++ |
| 227 | +++ |
| 228 | +++ |
| 229 | +++ |
| 230 | +++ |
| 231 | +++ |
| 232 | +++ |
| 233 | +++ |
| 234 | +++ |
| 235 | +++ |
| 236 | +++ |
| 237 | +++ |
| 238 | +++ |
| 239 | +++ |
| 240 | +++ |
| 241 | +++ |
| 242 | +++ |
| 243 | +++ |
| 244 | +++ |
| 245 | +++ |
| 246 | +++ |
| 247 | +++ |
| 248 | +++ |
| 249 | +++ |
| 250 | +++ |
| 251 | +++ |
| 252 | +++ |
| 253 | +++ |
| 254 | +++ |
| 255 | +++ |
| 256 | +++ |
| 257 | +++ |
| 258 | +++ |
| 259 | +++ |
| 260 | +++ |
| 261 | +++ |
| 262 | +++ |
| 263 | +++ |
| 264 | +++ |
| 265 | +++ |
| 267 | +++ |
| 268 | +++ |
| 269 | +++ |
| 270 | +++ |
| 271 | +++ |
| 272 | +++ |
| 273 | +++ |
| 274 | +++ |
| 275 | +++ |
| 276 | +++ |
| 277 | +++ |
| 278 | +++ |
| 279 | + |
| 280 | +++ |
| 281 | +++ |
| 282 | +++ |
| 283 | +++ |
| 284 | +++ |
| 285 | +++ |
| 286 | +++ |
| 287 | +++ |
| 288 | ++ |
| 289 | +++ |
| 290 | +++ |
| 291 | +++ |
| 292 | +++ |
| 293 | +++ |
| 294 | +++ |
| 295 | ++ |
| 296 | +++ |
| 297 | ++ |
| 298 | +++ |
| 299 | +++ |
| 300 | +++ |
| 301 | +++ |
| 302 | +++ |

TABLE 4

| Ex. | IC₅₀ |
|---|---|
| 1 | 1.38 |
| 2 | 0.081 |
| 3 | 0.047 |
| 4 | 0.039 |
| 5 | 0.216 |
| 6 | 0.141 |
| 7 | 0.350 |
| 8 | 0.216 |
| 9 | 0.4 |
| 10 | 1.30 |
| 11 | 0.531 |
| 12 | 0.588 |
| 13 | 1.75 |
| 14 | 3.12 |
| 15 | 3.19 |
| 16 | 9.83 |
| 17 | 4.19 |
| 18 | 1.45 |
| 19 | 0.153 |
| 20 | 3.19 |
| 21 | 6.10 |
| 22 | 10.9 |
| 23 | 1.88 |
| 24 | 7.17 |
| 25 | 3.57 |
| 26 | 0.445 |
| 27 | 0.291 |
| 28 | 0.373 |
| 29 | 0.392 |
| 30 | 15.1 |
| 31 | 2.41 |
| 32 | 1.95 |
| 33 | 1.28 |
| 34 | 0.137 |
| 35 | 0.366 |
| 36 | 2.72 |
| 37 | 1.32 |
| 38 | 0.449 |
| 39 | 0.090 |
| 40 | 8.78 |
| 41 | 0.068 |
| 42 | 5.24 |
| 43 | 0.368 |
| 44 | 1.26 |
| 45 | 6.25 |
| 46 | 1.54 |
| 47 | 4.70 |
| 48 | 0.238 |
| 49 | 3.01 |
| 50 | 6.44 |
| 51 | 9.07 |
| 52 | 2.08 |
| 53 | 7.83 |
| 54 | 1.55 |
| 55 | 0.861 |
| 56 | 7.92 |
| 57 | 5.92 |
| 58 | 3.09 |
| 59 | 18.3 |
| 60 | 8.68 |
| 61 | 2.75 |
| 62 | 12.1 |
| 63 | 16.5 |
| 64 | 6.40 |
| 65 | 6.86 |
| 66 | 26.2 |
| 67 | 8.75 |
| 68 | 28.6 |
| 69 | 0.643 |
| 70 | 0.191 |
| 71 | 6.96 |
| 72 | 0.511 |
| 73 | 11.5 |
| 74 | 1.76 |
| 75 | 12.3 |
| 76 | 16.2 |
| 77 | 3.72 |
| 78 | 0.208 |
| 79 | 0.015 |
| 80 | 1.06 |
| 81 | 2.56 |
| 82 | 2.22 |
| 83 | 0.521 |
| 84 | 5.98 |
| 85 | 0.992 |
| 86 | 2.81 |
| 87 | 5.39 |
| 88 | 3.61 |
| 89 | 6.84 |
| 90 | 0.034 |
| 91 | 3.21 |
| 92 | 4.46 |
| 93 | 2.28 |
| 94 | 0.369 |
| 95 | 0.320 |
| 96 | 1.33 |
| 97 | 1.22 |
| 98 | 0.349 |
| 99 | 0.598 |
| 100 | 0.114 |
| 101 | 0.672 |
| 102 | 0.169 |
| 103 | 22.3 |
| 104 | 1.87 |
| 105 | 0.119 |
| 106 | 0.156 |
| 107 | 2.82 |
| 108 | 0.018 |
| 109 | 0.036 |
| 110 | 0.012 |
| 111 | 2.42 |
| 112 | 13 |
| 113 | 4.18 |
| 114 | 1.99 |
| 115 | 15.4 |
| 116 | 4.16 |
| 117 | 19.8 |
| 118 | 22.4 |
| 119 | 30.1 |
| 120 | 24.8 |
| 121 | 12.1 |
| 122 | 29.9 |
| 123 | 34.9 |
| 124 | 27.7 |
| 125 | 26.7 |
| 126 | 15.4 |
| 127 | 1.67 |
| 128 | 12.0 |
| 129 | 7.20 |
| 130 | 1.23 |
| 131 | 7.70 |
| 132 | 29.7 |
| 133 | 9.24 |
| 134 | 1.41 |
| 134 | 32.5 |
| 135 | 1.22 |
| 137 | 21.9 |
| 138 | 20.5 |
| 139 | 26.4 |
| 140 | 0.889 |
| 141 | 1.23 |
| 142 | 1.78 |
| 143 | 1.41 |
| 144 | 3.91 |
| 145 | 2.66 |
| 146 | 2.74 |
| 147 | 8.12 |
| 148 | 1.79 |
| 149 | 6.58 |
| 150 | 2.10 |
| 151 | 9.06 |
| 152 | 21.6 |
| 153 | 32.9 |
| 154 | 4.33 |
| 155 | 3.61 |
| 156 | 4.39 |
| 157 | 9.98 |
| 158 | 5.90 |
| 159 | 1.77 |
| 160 | 4.2 |
| 161 | 6.12 |
| 162 | 3.54 |
| 163 | 6.66 |
| 164 | 6.01 |
| 165 | 1.63 |
| 166 | 4.84 |
| 167 | 0.146 |
| 168 | 2.83 |
| 169 | 0.605 |
| 170 | 1.12 |
| 171 | 11.0 |
| 172 | 23.7 |
| 173 | 2.30 |
| 174 | 10.9 |
| 175 | 0.672 |
| 176 | 2.95 |
| 177 | 4.41 |
| 178 | 1.05 |
| 179 | 2.24 |
| 180 | 0.508 |
| 181 | 0.143 |
| 182 | 3.14 |
| 183 | 8.08 |
| 184 | 0.651 |
| 185 | 0.469 |
| 186 | 5.23 |
| 187 | 0.871 |
| 188 | 1.29 |
| 189 | 3.76 |
| 190 | 0.691 |
| 191 | 1.15 |
| 192 | 0.309 |
| 193 | 0.240 |
| 194 | 0.390 |
| 195 | 1.01 |
| 196 | 0.516 |
| 197 | 1.25 |
| 198 | 1.50 |
| 199 | 1.37 |
| 200 | 3.62 |
| 201 | 1.32 |
| 202 | 0.187 |
| 203 | 0.972 |
| 204 | 0.646 |
| 205 | 0.306 |
| 206 | 1.26 |
| 207 | 0.487 |
| 208 | 0.709 |
| 209 | 3.56 |
| 210 | 0.822 |
| 211 | 1.18 |
| 212 | 0.589 |
| 213 | 1.34 |
| 214 | 7.50 |
| 215 | 6.86 |
| 216 | 3.98 |
| 217 | 0.862 |
| 218 | 9.18 |
| 219 | 1.62 |
| 220 | 0.020 |
| 221 | 0.014 |
| 222 | 0.011 |
| 223 | 0.055 |
| 224 | 0.049 |
| 225 | 0.019 |
| 226 | 0.031 |
| 227 | 0.127 |
| 228 | 0.021 |
| 229 | 0.032 |
| 230 | 0.051 |
| 231 | 0.050 |
| 232 | 0.028 |
| 233 | 0.045 |
| 234 | 0.078 |
| 235 | 0.850 |
| 236 | 1.12 |
| 237 | 0.061 |
| 238 | 0.414 |
| 239 | 0.037 |
| 240 | 1.22 |
| 241 | 0.134 |
| 242 | 1.29 |
| 243 | 0.095 |
| 244 | 0.075 |
| 245 | 0.938 |
| 246 | 0.006 |
| 247 | 0.010 |
| 248 | 0.003 |
| 249 | 0.015 |
| 250 | 0.014 |
| 251 | 0.550 |
| 252 | 0.454 |
| 253 | 0.597 |
| 254 | 0.434 |
| 255 | 0.148 |
| 256 | 0.395 |
| 257 | 0.392 |
| 258 | 0.526 |
| 259 | 0.437 |
| 260 | 0.205 |
| 261 | 0.224 |
| 262 | 0.253 |
| 263 | 0.281 |
| 264 | 0.312 |
| 265 | 0.335 |
| 267 | 0.359 |
| 268 | 0.192 |
| 269 | 0.087 |
| 270 | 0.286 |
| 271 | 0.029 |
| 272 | 0.014 |
| 273 | 0.059 |
| 274 | 0.023 |
| 275 | 0.070 |
| 276 | 0.079 |
| 277 | 0.021 |
| 278 | 1.79 |
| 279 | 22.4 |
| 280 | 2.37 |
| 281 | 0.108 |
| 282 | 1.05 |
| 283 | 0.101 |
| 284 | 0.050 |
| 285 | 0.165 |
| 286 | 1.95 |
| 287 | 0.251 |
| 288 | 15.4 |
| 289 | 0.027 |
| 290 | 3.73 |
| 291 | 0.037 |
| 292 | 3.94 |
| 293 | 0.134 |
| 294 | 0.203 |
| 295 | 12.8 |
| 296 | 0.345 |
| 297 | 14.4 |
| 298 | 0.367 |
| 299 | 0.096 |
| 300 | 3.43 |
| 301 | 0.027 |
| 302 | 0.539 |
| 303 | 1.44 |
| 304 | 2.56 |
| 305 | 0.662 |
| 306 | 0.577 |
| 307 | 0.898 |
| 308 | 0.025 |
| 309 | 0.696 |
| 310 | 1.06 |
| 311 | 1.41 |
| 312 | 1.02 |
| 313 | 0.888 |
| 314 | 0.784 |
| 315 | 8.92 |
| 316 | 0.704 |
| 317 | 8.51 |
| 318 | 0.008 |
| 319 | 3.05 |
| 320 | 0.014 |
| 321 | 0.009 |
| 322 | 0.038 |
| 323 | >10 |
| 324 | 0.027 |
| 325 | >1 |
| 326 | 0.013 |
| 327 | >1 |
| 328 | 0.067 |
| 329 | 0.930 |
| 330 | 1.02 |
| 331 | 0.970 |
| 332 | 0.768 |
| 333 | 0.895 |
| 334 | 7.51 |
| 335 | 2.08 |
| 336 | 23.2 |
| 337 | 7.47 |
| 338 | 1.58 |
| 339 | 8.29 |
| 340 | 7.87 |
| 341 | 2.41 |
| 342 | 17.1 |
| 343 | 7.50 |
| 344 | 27.6 |
| 345 | 15.1 |
| 346 | 1.57 |
| 347 | 21.5 |
| 348 | 1.32 |
| 349 | 4.76 |
| 350 | 3.59 |
| 351 | 4.23 |
| 352 | 6.08 |
| 353 | 5.422 |
| 354 | 24.0 |
| 355 | 5.02 |
| 356 | 2.30 |
| 357 | 5.21 |
| 358 | 1.29 |
| 359 | 0.603 |
| 360 | 3.46 |
| 361 | 0.849 |
| 362 | 1.45 |
| 363 | 6.58 |
| 364 | 2.56 |
| 365 | 2.14 |
| 366 | 19.0 |
| 367 | 11.7 |
| 368 | 1.60 |
| 369 | 6.51 |
| 370 | 5.97 |
| 371 | 1.46 |
| 372 | 22.2 |
| 373 | 1.59 |
| 374 | 9.45 |
| 375 | 11.1 |
| 376 | 1.83 |
| 377 | 2.25 |
| 378 | 4.98 |
| 379 | 8.92 |
| 380 | 11.4 |
| 381 | 12.1 |
| 382 | 8.79 |
| 383 | 1.23 |
| 384 | 11.6 |
| 385 | 1.82 |
| 386 | 1.45 |
| 387 | 5.29 |
| 389 | 0.546 |
| 390 | 9.84 |
| 391 | 22.4 |
| 392 | 5.70 |
| 393 | 7.89 |
| 394 | 5.21 |
| 395 | 1.38 |
| 396 | 1.60 |
| 397 | 2.00 |
| 398 | 1.91 |
| 399 | 1.35 |
| 400 | 0.612 |
| 401 | 1.89 |
| 402 | 9.27 |
| 403 | 2.32 |
| 404 | 3.89 |
| 405 | 18.0 |
| 406 | 16.1 |
| 407 | 4.28 |
| 408 | 0.827 |
| 409 | 1.37 |
| 410 | 1.52 |
| 411 | 1.34 |
| 412 | 3.17 |
| 413 | 2.02 |
| 414 | 1.26 |
| 415 | 21.0 |
| 416 | 20.8 |
| 417 | 11.7 |
| 418 | 3.48 |
| 419 | 0.769 |
| 420 | 3.80 |
| 421 | 2.73 |
| 422 | 3.08 |
| 423 | 18.4 |
| 424 | 0.700 |
| 425 | 2.85 |
| 427 | 6.28 |
| 429 | 1.66 |
| 430 | 0.634 |

Unless otherwise defined, all technical and scientific terms used herein have the same meaning as commonly understood by one of ordinary skill in the art to which this disclosure belongs.

The disclosure illustratively described herein may suitably be practiced in the absence of any element or elements, limitation or limitations, not specifically disclosed herein. Thus, for example, the terms “comprising,” “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claims.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A compound of Formula I: 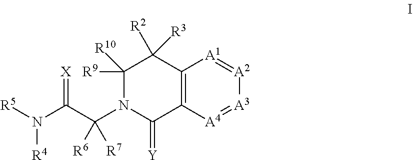
or a pharmaceutically acceptable salt, isotopically enriched analog, stereoisomer, mixture of stereoisomers, or prodrug thereof, wherein:
X is O or S;
Y is O or S;
A¹, A², A³, and A⁴ are each independently N, CH, or CR¹; provided at least one of A¹, A², A³, and A⁴ is CR¹;
each R¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —C(O)R¹¹, —C(O)OR¹¹, —S(O)₀₋₂R¹¹, —NR¹¹S(O)₀₋₂—R¹¹, —S(O)₀₋₂N(R¹¹)₂, —NR¹¹S(O)₀₋₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹; or
any two adjacent R¹ together with the atoms to which they are attached form a cycloalkyl, heterocyclyl, aryl, or heteroaryl ring; wherein the cycloalkyl, heterocyclyl, aryl, or heteroaryl is independently optionally substituted with one to eight Z¹;
R² is C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, —NO₂, —SF₅, —OR¹¹, —N(R¹¹)₂, —C(O)R¹¹, —C(O)OR¹¹, —S(O)₀₋₂—R¹¹, —NR¹¹S(O)₀₋₂—R¹¹, —S(O)₀₋₂N(R¹¹)₂, —NR¹¹S(O)₀₋₂N(R¹¹)₂, —NR¹¹C(O)N(R¹¹)₂, —NR¹¹C(O)OR¹¹, —NR¹¹C(O)R¹¹, —OC(O)R¹¹, —OC(O)N(R¹¹)₂, —C(O)N(R¹¹)₂, halo, or cyano; wherein the C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is optionally substituted with one to eight Z¹;
R³ is hydrogen, halo, cyano, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is optionally substituted with one to eight Z¹; or
R² and R³ together form a C₃₋₁₀ cycloalkyl or heterocyclyl ring; wherein the C₃₋₁₀ cycloalkyl or heterocyclyl is optionally substituted with one to eight Z¹;
R⁴ is hydrogen, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is optionally substituted with one to eight Z¹; or
R⁵ is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is optionally substituted with one to eight Z¹; or
R⁴ and R⁵ together form a heterocyclyl or heteroaryl ring optionally substituted with one to eight Z¹;
R⁶ is hydrogen, halo, cyano, C₁₋₆ alkyl, C₂₋₆ alkenyl, C₂₋₆ alkynyl, C₁₋₆haloalkyl, C₂₋₆ heteroalkyl, C₃₋₁₀ cycloalkyl, or heterocyclyl; wherein the C₁₋₆ alkyl, C₂₋₆ alkenyl, C₂₋₆ alkynyl, C₁₋₆ haloalkyl, C₂₋₆ heteroalkyl, C₃₋₁₀ cycloalkyl, or heterocyclyl may further be optionally substituted with one to five Z^{1b};
R⁷ is hydrogen, halo, cyano, hydroxy, C₁₋₆ alkyl, C₂₋₆ alkenyl, C₂₋₆ alkynyl, C₁₋₆ haloalkyl, C₂₋₆ heteroalkyl, C₃₋₁₀ cycloalkyl, or heterocyclyl; wherein the C₁₋₆ alkyl, C₂₋₆ alkenyl, C₂₋₆ alkynyl, C₁₋₆ haloalkyl, C₂₋₆ heteroalkyl, C₃₋₁₀ cycloalkyl, or heterocyclyl, or may further be optionally substituted with one to five Z^{1b};
or R⁶ and R⁷ join to form a C₃₋₁₀ cycloalkyl or heterocyclyl ring; wherein the C₃₋₁₀ cycloalkyl or heterocyclyl ring may further be optionally substituted with one to five Z^{1b};
R⁹ and R¹⁰ are each independently hydrogen, halo, cyano, C₁₋₆ alkyl, or C₁₋₆ haloalkyl, wherein each C₁₋₆ alkyl or C₁₋₆ haloalkyl is independently optionally substituted with one to five Z¹; or
R⁹ and R¹⁰ together form a C₃₋₁₀ cycloalkyl or heterocyclyl ring; wherein the C₃₋₁₀ cycloalkyl or heterocyclyl is optionally substituted with one to eight Z¹;
each Z¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹, —C(O)R¹¹, —C(O)OR¹¹, —S(O)₀₋₂R¹¹, —NR¹¹S(O)₀₋₂—R¹¹, —S(O)₀₋₂N(R¹¹)₂, —NR¹¹S(O)₀₋₂N(R¹¹)₂, —NR¹¹C(O)N(R¹¹)₂, —C(O)N(R¹¹)₂, —NR¹¹C(O)R¹¹, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each R¹¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹¹ is independently optionally substituted with one to five Z^{1a};
each Z^{1a} is independently hydroxy, halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, —N(R¹³)₂, —OR¹³, —C(O)R¹³, —C(O)OR¹³, —S(O)₀₋₂R¹³, —NR¹³S(O)₀₋₂—R¹³, —S(O)₀₋₂N(R¹³)₂, —NR¹³S(O)₀₋₂N(R¹³)₂, —NR¹³C(O)N(R¹³)₂, —C(O)N(R¹³)₂, —NR¹³C(O)R¹³, —OC(O)N(R¹³)₂, or —NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹³ is independently optionally substituted with one to five Z^{1b};
each Z^{1b} is independently halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, —N(C₁₋₆ alkyl)-, —N(C₂₋₆ alkenyl)-, —N(C₂₋₆ alkynyl)-, —N(C₁₋₆ haloalkyl)-, —N(C₃₋₁₀ cycloalkyl)-, —N(heterocyclyl)-, —N(aryl)-, —N(heteroaryl)-, —C(O)—, —C(O)O—, —C(O)NH—, —C(O)N(C₁₋₆ alkyl)-, —C(O)N(C₂₋₆ alkenyl)-, —C(O)N(C₂₋₆ alkynyl)-, —C(O)N(C₁₋₆ haloalkyl)-, —C(O)N(C₃₋₁₀ cycloalkyl)-, —C(O)N(heterocyclyl)-, —C(O)N(aryl)-, —C(O)N(heteroaryl)-, —NHC(O)—, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five hydroxy, halo, cyano, hydroxy, —SH, —NH₂, —NO₂, —SF₅, C₁₋₆ alkyl, C₂₋₆ alkenyl, C₂₋₆ alkynyl, C₁₋₆haloalkyl, C₁₋₆ alkoxy, C₁₋₆ haloalkoxy, C₃₋₁₀ cycloalkyl, heterocyclyl, aryl, or heteroaryl.

2. The compound of claim 1, wherein X is O.

3. The compound of claim 1, wherein Y is O

4. The compound of claim 1, wherein X and Y are O.

5. The compound of claim 1, wherein X is O and Y is S.

6. The compound of claim 1, wherein X is S and Y is O.

7. The compound of claim 1, wherein X and Y are S.

8. The compound of claim 1, wherein the compound is represented by Formula IA:

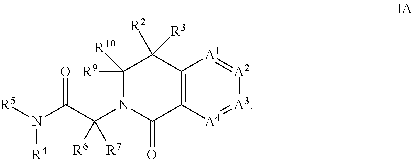

9. The compound of any one of claims 1-8, wherein at least one of A¹, A², A³, and A⁴ is N.

10. The compound of claim 1, wherein the compound is represented by Formula IB:

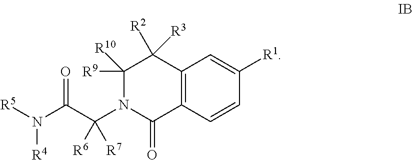

11. The compound of any one of claims 1-10, wherein R⁴ is hydrogen or C₁₋₆ alkyl.

12. The compound of any one of claims 1-11, wherein R⁴ is hydrogen or methyl.

13. The compound of any preceding claim, wherein R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted with one to five Z¹.

14. The compound of any one of claims 1-13, wherein R⁵ is (1-(2,2-difluoroethyl)cyclobutyl)methyl, (1-methyl-1H-imidazol-2-yl)methyl, (1-methyl-1H-pyrazol-4-yl)methyl, (1-methyl-1H-pyrazol-5-yl)methyl, (1R,2R,4S)-7-oxabicyclo[2.2.1]heptan-2-yl, (1S,2R,4R)-7-oxabicyclo[2.2.1]heptan-2-yl, (2-(trifluoromethyl)pyridin-3-yl)methyl, [1,2,4]triazolo[1,5-a]pyridin-2-yl, [1,2,4]triazolo[4,3-a]pyridin-6-yl, [1,2,4]triazolo[1,5-a]pyrazin-2-yl, 7-chloro-[1,2,4]triazolo[1,5-a]pyridin-2-yl, 7-(trifluoromethyl)-[1,2,4]triazolo[1,5-a]pyridin-2-yl, 6-chloro-[1,2,4]triazolo[1,5-a]pyridin-2-yl, 6-fluoro-[1,2,4]triazolo[1,5-a]pyridin-2-yl, 6-methoxy-[1,2,4]triazolo[1,5-a]pyridin-2-yl, 1-(2-hydroxy-2-methylpropyl)cyclopropyl, 1-(2-methoxyethyl)-1H-pyrazol-4-yl, 1-(2-methoxyethyl)-3-piperidyl, 1-(6-chloropyridazin-3-yl)piperidin-4-yl, 1-(hydroxymethyl)cyclopropyl, 1-(methoxycarbonyl)piperidin-3-yl, 1,1-dioxidothietan-3-yl, 1,3,5-triazin-2-yl, 1,3-dimethyl-1H-pyrazol-5-yl, 1,6-naphthyridin-2-yl, 1,7-naphthyridin-6-yl, 1,8-naphthyridin-2-yl, 1-bicyclo[2.2.2]octanyl, 1-cyclobutylpiperidin-3-yl, 1-cyclopropylpiperidin-3-yl, 1-ethyl-6-oxo-3-piperidyl, 1-ethylpiperidin-3-yl, 1H-benzo[d][1,2,3]triazol-5-yl, 1H-benzo[d]imidazol-2-yl, 1H-benzo[d]imidazol-6-yl, 1H-indazol-3-yl, 1H-indazol-5-yl, 1H-indazol-6-yl, 1H-indol-6-yl, 1H-pyrrolo[2,3-b]pyridin-5-yl, 1-methyl-1H-1,2,4-triazol-5-yl, 1-methyl-1H-benzo[d]imidazol-5-yl, 1-methyl-1H-indazol-5-yl, 1-methyl-1H-pyrazolo[4,3-b]pyridin-5-yl, 1-methyl-2-oxabicyclo[2.1.1]hexan-4-yl, 1-methyl-2-oxo-4-piperidyl, 1-methyl-5-oxo-pyrrolidin-3-yl, 1-methyl-6-oxo-3-piperidyl, 1-methyl-6-oxo-3-pyridyl, 1-phenyl-1H-pyrazol-5-yl, 1-phenylcyclopropyl, 2-(1H-imidazol-1-yl)ethyl, 2-(4-fluorophenyl)-2-hydroxyethyl, 2-(difluoromethoxy)phenyl, 2-(methylsulfonamido)ethyl, 2-(methylsulfonyl)ethyl, 2,2-difluorobenzo[d][1,3]dioxol-5-yl, 2,3-dihydro-1H-inden-2-yl, 2,3-dihydrobenzofuran-5-yl, 2,6-dimethylpyrimidin-4-yl, 2-chloro-4-(methylsulfonyl)phenyl, 2-cyanopropan-2-yl, 2-cyclopropyltetrahydropyran-4-yl, 2-hydroxy-2-methyl-propyl, 2-methyl-2H-pyrazolo[4,3-b]pyridin-5-yl, 2-methylbenzo[d]thiazol-6-yl, 2-morpholinoethyl, 2-oxabicyclo[2.2.2]octan-4-yl, 2-oxaspiro[3.3]heptan-6-yl, 3-(1-hydroxy-1-methyl-ethyl)-1-bicyclo[1.1.1]pentanyl, 3-(2-methylthiazol-4-yl)phenyl, 3-(difluoromethoxy)cyclobutyl, 3-(difluoromethyl)cyclobutyl, 3-(hydroxymethyl)cyclobutyl, 3-(trifluoromethyl)-1-bicyclo[1.1.1]pentanyl, 3-(trifluoromethyl)cyclobutyl, 3,3,3-trifluoropropyl, 3,3-difluorocyclobutyl, 3,4-dimethylisoxazol-5-yl, 3,5-difluoro-2-pyridyl, 3-cyano-1-bicyclo[1.1.1]pentanyl, 3-cyanocyclobutyl, 3-cyclopropyl-1H-pyrazol-5-yl, 3-cyclopropyl-1-methyl-1H-pyrazol-5-yl, 3-fluoro-5-(1H-pyrazol-1-yl)pyridin-2-yl, 3-fluoro-5-(trifluoromethyl)pyridin-2-yl, 3-fluoro-5-formylpyridin-2-yl, 3-fluoropyridin-4-yl, 3-hydroxy-3-(trifluoromethyl)cyclobutyl, 3-hydroxy-3-methylbutyl, 3-hydroxy-3-methylcyclobutyl, 3-hydroxycyclohexyl, 3-methyl-1-phenyl-1H-pyrazol-5-yl, 3-methylcyclobutyl, 4-(1H-tetrazol-5-yl)phenyl, 4-(2-methylthiazol-4-yl)pyrimidin-2-yl, 4,4-difluorocyclohexyl, 4,5,6,7-tetrahydro-1H-indazol-6-yl, 4,5,6,7-tetrahydropyrazolo[1,5-a]pyridin-5-yl, 4,5-dimethylpyrimidin-2-yl, 4,6-dimethylpyridin-2-yl, 4-cyanopyrimidin-2-yl, 4-hydroxy-1-bicyclo[2.2.2]octanyl, 4-methylpyridin-2-yl, 5-(difluoromethoxy)-2-pyridyl, 5-(difluoromethyl)pyridin-2-yl, 5-(pyridin-2-yl)pyrimidin-2-yl, 5-(trifluoromethyl)pyrimidin-2-yl, 5-(difluoromethoxy)pyrimidin-2-yl, 5,7-dihydrofuro[3,4-d]pyrimidin-2-yl, 5-chloro-3-fluoropyridin-2-yl, 5-chloropyridin-2-yl, 5-chloropyrimidin-2-yl, 5-cyano-3-fluoropyridin-2-yl, 5-cyanobenzo[d]oxazol-2-yl, 5-cyanopyridin-2-yl, 5-cyanopyrimidin-2-yl, 5-cyclopropylpyrimidin-2-yl, 5-cyclobutylpyrimidin-2-yl, 5-ethylpyrimidin-2-yl, 5-fluoro-4-methylpyrimidin-2-yl, 5-cyano-4-methylpyrimidin-2-yl, 5-fluoropyridin-2-yl, 5-fluoropyrimidin-2-yl, 5-fluoropyrimidin-4-yl, 5-iodopyrimidin-2-yl, 5-methoxypyrimidin-2-yl, 5-methyl-2-oxo-1,2-dihydropyridin-3-yl, 5-methylpyrimidin-2-yl, 5-pyrazol-1-ylpyrimidin-2-yl, 5-(tetrahydrofuran-3-yl)pyrimidin-2-yl, 5-(1-methyl-1H-pyrazol-4-yl)pyrimidin-2-yl, 6,7-dihydro-5H-pyrrolo[1,2-b][1,2,4]triazol-2-yl, 5-fluorothiazol-2-yl, 6-chloropyridazin-3-yl, 6-fluorobenzo[d]oxazol-2-yl, 6-cyanobenzo[d]oxazol-2-yl, 6-methylpyrazin-2-yl, 6-methylpyridin-2-yl, 6-oxo-1,6-dihydropyrimidin-2-yl, benzo[d]oxazol-2-yl, benzo[d]oxazol-5-yl, benzo[d]thiazol-5-yl, benzo[d]thiazol-6-yl, cyclobutylmethyl, imidazo[1,2-a]pyrazin-6-yl, imidazo[1,2-a]pyridin-5-yl, imidazo[1,2-a]pyridin-8-yl, imidazo[1,2-b]pyridazin-6-yl, imidazo[1,5-a]pyridin-6-yl, isoquinolin-4-yl, isoquinolin-6-yl, isoquinolin-7-yl, isoquinolin-8-yl, isoxazolo[4,5-b]pyridin-5-yl, isoxazolo[5,4-b]pyridin-6-yl, oxazol-2-ylmethyl, oxazolo[4,5-b]pyridin-2-yl, oxazolo[4,5-c]pyridin-2-yl, oxazolo[5,4-b]pyridin-2-yl, oxazolo[5,4-c]pyridin-2-yl, oxetan-3-ylmethyl, phenyl, pyrazolo[1,5-a]pyrimidin-5-yl, pyridin-4-ylmethyl, pyrimidin-2-yl, quinazolin-2-yl, quinolin-2-yl, quinolin-3-yl, quinolin-5-yl, quinolin-6-yl, spiro[2.3]hexan-5-yl, [1,2,4]triazolo[1,5-a]pyrazin-8-yl, [1,2,4]triazolo[4,3-a]pyrazin-8-yl, [1,3]thiazolo[5,4-d]pyrimidin-5-yl, 1-(1-methylpyrazol-3-yl)pyrrolidin-3-yl, 1-(1-methylpyrazol-4-yl)piperidin-3-yl, 1-(1-methylpyrazol-4-yl)pyrrolidin-3-yl, 1-(2,2,2-trifluoroethyl)-1,2,4-triazol-3-yl, 1-(2,2,2-trifluoroethyl)piperidin-4-yl, 1-(2,2-difluoroethyl)piperidin-4-yl, 1-(3,3,3-trifluoropropyl)piperidin-4-yl, 1-(oxetan-3-yl)piperidin-3-yl, 1-(oxetan-3-yl)pyrrolidin-3-yl, 1,2,4-benzotriazin-3-yl, 1,2-benzothiazol-6-yl, 1,2-benzoxazol-3-yl, 1,5-dimethyl-1,2,4-triazol-3-yl, 1,7-naphthyridin-8-yl, 1-azabicyclo[2.2.2]octan-3-yl, 1-benzylpyrrolidin-3-yl, 1-cyclopropyl-1,2,4-triazol-3-yl, 1-ethyl-1-azaspiro[3.3]heptan-6-yl, 1-ethylpyrrolidin-3-yl, 1-methyl-1,2,4-triazol-3-yl, 1-methyl-2-oxopyrrolidin-3-yl, 1-methyl-6-oxopyridazin-3-yl, 1-methylpiperidin-3-yl, 1-methylpyrazolo[3,4-d]pyrimidin-6-yl, 1-phenyl-1,2,4-triazol-3-yl, 1-propan-2-yl-1,2,4-triazol-3-yl, 1-pyridazin-3-ylpiperidin-4-yl, 1-pyridin-2-ylpiperidin-4-yl, 1-pyridin-3-ylpiperidin-4-yl, 1-pyrimidin-2-ylpiperidin-4-yl, 2-methylimidazo[1,2-b]pyridazin-6-yl, 2-oxopyrrolidin-3-yl, 3-(1H-pyrazol-5-yl)cyclobutyl, 3-(methoxymethyl)cyclobutyl, 3-chloro-5-cyanopyridin-2-yl, 3-cyano-5-fluoropyridin-2-yl, 3-fluoro-5-methylpyridin-2-yl, 3-fluoroimidazo[1,2-a]pyridin-2-yl, 3-fluoropyrazolo[1,5-a]pyridin-2-yl, 3-methoxy-3-methylcyclobutyl, 3-methoxypyridin-2-yl, 3-methylimidazo[1,2-b]pyridazin-6-yl, 3-methylpyrazolo[1,5-a]pyridin-2-yl, 3-phenylcyclobutyl, 3-phenylmethoxycyclobutyl, 4,4-dimethyl-5H-1,3-oxazol-2-yl, 4,5,6,7-tetrahydro-1,3-benzoxazol-2-yl, 4-cyano-1,3-benzoxazol-2-yl, 4-methoxypyrimidin-2-yl, 4-methyl-3-oxopyrazin-2-yl, 4-methyl-4-azaspiro[2.5]octan-7-yl, 4-methyl-5-oxopyrazin-2-yl, 5-(2,2-difluorocyclopropyl)pyrimidin-2-yl, 5-(2,3-dihydrofuran-4-yl)pyrimidin-2-yl, 5-(difluoromethyl)-3-fluoropyridin-2-yl, 5-(methoxymethoxy)pyrimidin-2-yl, 5-(oxetan-3-yl)pyrimidin-2-yl, 5-(oxolan-2-yl)pyrimidin-2-yl, 5-(trifluoromethyl)-1,3-benzoxazol-2-yl, 5,5-dimethyl-4H-1,3-oxazol-2-yl, 5,6,7,8-tetrahydro-[1,2,4]triazolo[1,5-a]pyridin-2-yl, 5,6-dihydrofuro[2,3-d]pyrimidin-2-yl, 5-cyano-3-fluoro-4-methylpyridin-2-yl, 5-cyano-3-fluoro-6-methylpyridin-2-yl, 5-cyano-3-methylpyridin-2-yl, 5-fluoro-2-methoxypyrimidin-4-yl, 5-fluoro-6-methoxypyrimidin-4-yl, 5-methyl-1-phenyl-1,2,4-triazol-3-yl, 5-pyrrolidin-1-ylpyrimidin-2-yl, 6-(difluoromethoxy)pyridin-3-yl, 6-(trifluoromethyl)-1,3-benzoxazol-2-yl, 6,7-dihydro-4H-pyrazolo[5,1-c][1,4]oxazin-2-yl, 6,8-dihydro-5H-pyrano[3,4-d]pyrimidin-2-yl, 6-cyano-4-fluoropyridin-3-yl, 6-cyanopyridin-3-yl, 6-fluoro-1,3-benzoxazol-2-yl, 6-fluoropyrazolo[1,5-a]pyrimidin-5-yl, 6-methoxypyridin-3-yl, 7,8-dihydro-5H-pyrano[4,3-d]pyrimidin-2-yl, 7-methylpyrazolo[1,5-a]pyrimidin-5-yl, 8-chloro-[1,2,4]triazolo[1,5-a]pyridin-2-yl, 1-(ethoxycarbonyl)piperidin-4-yl, imidazo[1,2-a]pyrazin-8-yl, imidazo[1,2-a]pyridin-2-yl, imidazo[1,2-a]pyrimidin-7-yl, imidazo[1,2-c]pyrimidin-5-yl, pyrazin-2-yl, pyrazolo[1,5-a]pyridin-2-yl, pyridazin-4-yl, 1-(tert-butoxycarbonyl)-1-azaspiro[3.3]heptan-6-yl, or 6-oxo-1,6-dihydropyridazin-3-yl.

15. The compound of any one of claims 1-10, wherein R⁴ and R⁵ together form a heterocyclyl or heteroaryl ring optionally substituted with one to eight Z¹.

16. The compound of any preceding claim, wherein R⁹ is hydrogen or C₁₋₆ alkyl.

17. The compound of any preceding claim, wherein R⁹ is hydrogen or methyl and R¹⁰ is hydrogen.

18. The compound of claim 1, wherein the compound is represented by Formula II: 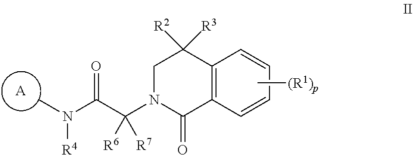
wherein:
p is 1, 2, 3 or 4; and
ring A is C₃₋₁₀ cycloalkyl, heterocyclyl, aryl, or heteroaryl; wherein the C₃₋₁₀ cycloalkyl, heterocyclyl, aryl, or heteroaryl is optionally substituted with one to eight Z¹.

19. The compound of claim 1, wherein the compound is represented by Formula III: 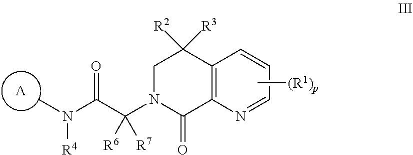
wherein:
p is 1, 2, 3 or 4; and
ring A is C₃₋₁₀ cycloalkyl, heterocyclyl, aryl, or heteroaryl; wherein the C₃₋₁₀ cycloalkyl, heterocyclyl, aryl, or heteroaryl is optionally substituted with one to eight Z¹.

20. The compound of any one of claims 1-14 or 16-19, wherein R⁴ is hydrogen or methyl.

21. The compound of any preceding claim, wherein R⁶ is hydrogen.

22. The compound of any preceding claim, wherein R⁷ is hydrogen.

23. The compound of any one of claims 1-22, wherein R⁶ and R⁷ join to form a C₃₋₁₀ cycloalkyl.

24. The compound of claim 1, wherein the compound is represented by Formula IV: 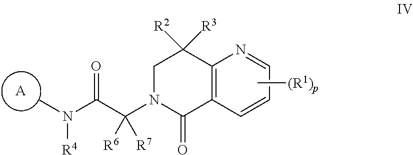
wherein:
p is 1, 2, 3 or 4; and
ring A is C₃₋₁₀ cycloalkyl, heterocyclyl, aryl, or heteroaryl; wherein the C₃₋₁₀ cycloalkyl, heterocyclyl, aryl, or heteroaryl is optionally substituted with one to eight Z¹.

25. The compound of claim 1, wherein the compound is represented by Formula V: 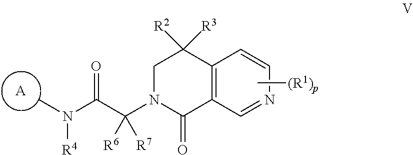
wherein:
p is 1, 2, 3 or 4; and
ring A is C₃₋₁₀ cycloalkyl, heterocyclyl, aryl, or heteroaryl; wherein the C₃₋₁₀ cycloalkyl, heterocyclyl, aryl, or heteroaryl is optionally substituted with one to eight Z¹.

26. The compound of claim 1, wherein the compound is represented by Formula VI: 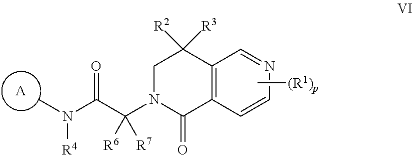
wherein:
p is 1, 2, 3 or 4; and
ring A is C₃₋₁₀ cycloalkyl, heterocyclyl, aryl, or heteroaryl; wherein the C₃₋₁₀ cycloalkyl, heterocyclyl, aryl, or heteroaryl is optionally substituted with one to eight Z¹.

27. The compound of claim 1, wherein the compound is represented by Formula VII:

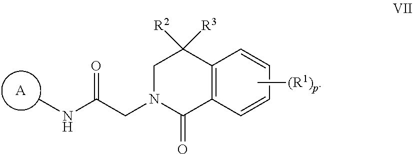

28. The compound of any preceding claim, wherein R² and R³ together form a C₃₋₁₀ cycloalkyl or heterocyclyl ring; wherein the C₃₋₁₀ cycloalkyl or heterocyclyl is independently optionally substituted with one to eight Z¹.

29. The compound of any preceding claim, wherein R² and R³ together form a C₃₋₁₀ cycloalkyl optionally substituted with halo, cyano, C₁₋₆ alkyl, or C₁₋₆ haloalkyl.

30. The compound of any one of claims 1-27, wherein R² is C₁₋₆ alkyl, C₁₋₆ haloalkyl, or —OR¹¹, wherein R¹¹ is C₁₋₆ alkyl optionally substituted with one to five Z^{1a}.

31. The compound of any one of claims 1-27 or 30, wherein R³ is hydrogen or C₁₋₆ alkyl.

32. The compound of any one of claims 1-27 or 30-31, wherein R² is C₁₋₆ alkyl or C₁₋₆ haloalkyl, and R³ is hydrogen or C₁₋₆ alkyl.

33. The compound of any one of claims 1-27 or 31-32, wherein R² and R³ are C₁₋₆ alkyl.

34. The compound of claim 1, wherein the compound is represented by Formula VIII:

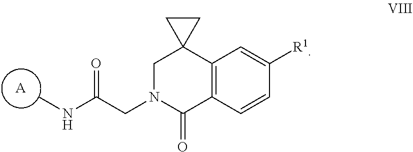

35. The compound of any preceding claim, wherein each R¹ is independently halo, cyano, C₁₋₆ alkyl, C₂₋₆ alkenyl, C₂₋₆ alkynyl, C₁₋₆ alkoxy, C₁₋₆ haloalkyl, C₁₋₆ haloalkoxy, C₃₋₁₀ cycloalkyl, or heterocyclyl, wherein the C₁₋₆ alkyl, C₂₋₆ alkenyl, and C₃₋₁₀ cycloalkyl are independently optionally substituted with one to eight Z¹; or any two adjacent R¹ together with the atoms to which they are attached form a cycloalkyl, heterocyclyl, aryl, or heteroaryl ring.

36. The compound of any preceding claim, wherein each R¹ is independently fluoro, bromo, chloro, iodo, cyano, ethyl, vinyl, difluoromethyl, trifluoromethyl, 1-fluoroethyl, 1,1-difluoroethyl, methoxy, fluoromethoxy, difluoromethoxy, cyclopropyl, cyclobutyl, cyclopropylmethyl, oxetan-3-yl, 2,2-difluorocycloprop-1-yl, 1-cyanocyclopropyl, and 1-methylcyclopropyl, 1-fluoro-2-(trifluoromethyl)cyclopropyl, ethynyl, 1-fluorovinyl, 1-fluorocyclopropyl, 2-fluorocyclopropyl, or 1,2-difluorocyclopropyl; or two adjacent R¹ together with the atoms to which they are attached form a thiophene.

37. A compound selected from Table 1, Table 1A, or Table 2, or a pharmaceutically acceptable salt, stereoisomer, mixture of stereoisomers, or prodrug thereof.

38. A pharmaceutical composition comprising a compound of any preceding claim, or a pharmaceutically acceptable salt, stereoisomer, mixture of stereoisomers, or prodrug thereof, and a pharmaceutically acceptable carrier.

39. A method for treating a disease or condition mediated, at least in part, by NLRP3, the method comprising administering an effective amount of the pharmaceutical composition of claim 38 to a subject in need thereof.

40. The method of claim 39, wherein the disease or condition is Alzheimer disease, atherosclerosis, asthma, allergic airway inflammation, cryopyrin-associated periodic syndromes, gout, inflammatory bowel disease and related disorders, nonalcoholic fatty liver disease (NAFLD), nonalcoholic steatohepatitis (NASH), hypertension, myocardial infarction, multiple sclerosis, experimental autoimmune encephalitis, oxalate-induced nephropathy, hyperinflammation following influenza infection, graft-versus-host disease, stroke, silicosis, type 1 diabetes, obesity-induced inflammation or insulin resistance, rheumatoid arthritis, myelodysplastic syndrome, contact hypersensitivity, joint inflammation triggered by chikungunya virus, or traumatic brain injury.

41. The method of claim 40, wherein the disease is nonalcoholic fatty liver disease (NAFLD) or nonalcoholic steatohepatitis (NASH).

42. The method of claim 40, wherein the disease is Alzheimer's disease.

43. Use of a compound of any one of claims 1-37, or a pharmaceutically acceptable salt, stereoisomer, mixture of stereoisomers, or prodrug thereof, for treating a disease or condition mediated, at least in part, by NLRP3.

44. The use of claim 43, wherein the disease or condition is Alzheimer disease, atherosclerosis, asthma, allergic airway inflammation, cryopyrin-associated periodic syndromes, gout, inflammatory bowel disease and related disorders, nonalcoholic fatty liver disease (NAFLD), nonalcoholic steatohepatitis (NASH), hypertension, myocardial infarction, multiple sclerosis, experimental autoimmune encephalitis, oxalate-induced nephropathy, hyperinflammation following influenza infection, graft-versus-host disease, stroke, silicosis, type 1 diabetes, obesity-induced inflammation or insulin resistance, rheumatoid arthritis, myelodysplastic syndrome, contact hypersensitivity, joint inflammation triggered by chikungunya virus, or traumatic brain injury.

45. A compound of any one of claims 1-37, or a pharmaceutically acceptable salt, stereoisomer, mixture of stereoisomers, or prodrug thereof, for use in therapy.

46. A compound of any one of claims 1-37, or a pharmaceutically acceptable salt, stereoisomer, mixture of stereoisomers, or prodrug thereof, for use in treating Alzheimer disease.

47. A compound of any one of claims 1-37, or a pharmaceutically acceptable salt, stereoisomer, mixture of stereoisomers, or prodrug thereof, for use in treating nonalcoholic fatty liver disease (NAFLD) or nonalcoholic steatohepatitis (NASH).

48. The use of a compound of claims 1-37, or a pharmaceutically acceptable salt, stereoisomer, mixture of stereoisomers, or prodrug thereof, for the manufacture of a medicament for treating a neurodegenerative disease, treating Alzheimer disease, atherosclerosis, asthma, allergic airway inflammation, cryopyrin-associated periodic syndromes, gout, inflammatory bowel disease and related disorders, nonalcoholic fatty liver disease (NAFLD), nonalcoholic steatohepatitis (NASH), hypertension, myocardial infarction, multiple sclerosis, experimental autoimmune encephalitis, oxalate-induced nephropathy, hyperinflammation following influenza infection, graft-versus-host disease, stroke, silicosis, type 1 diabetes, obesity-induced inflammation or insulin resistance, rheumatoid arthritis, myelodysplastic syndrome, contact hypersensitivity, joint inflammation triggered by chikungunya virus, or traumatic brain injury.

49. A process for preparing the compound of Formula I according to claim 1, comprising contacting a compound of Formula I-1 with a compound of Formula I-2: 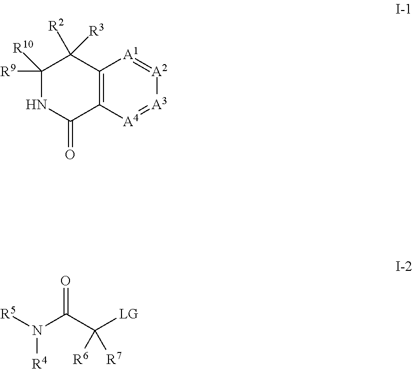
under conditions suitable to provide a compound of Formula I, wherein LG is a leaving group, and the remaining variables are defined according to claim 1.

50. A process for preparing the compound of Formula I according to claim 1, comprising contacting a compound of Formula I-4 with a compound of Formula I-5: 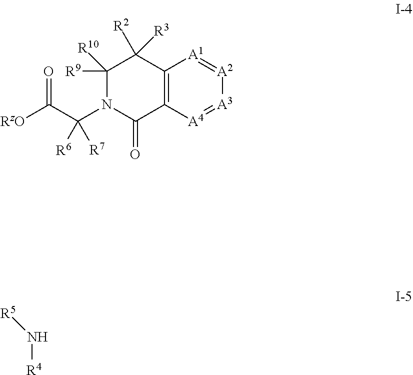
under conditions suitable to provide a compound of Formula I, wherein R^{z} is hydrogen or C₁₋₆ alkyl, and the remaining variables are defined according to claim 1.
